(19) United States
(12) Patent Application Publication
(10) Pub. No.: US 2024/0383905 A1
(43) Pub. Date: Nov. 21, 2024

(54) FGFR INHIBITORS AND METHODS OF USE THEREOF

(71) Applicants: 3H PHARMACEUTICALS CO., LTD., Shanghai (CN); 3H (SUZHOU) PHARMACEUTICALS CO., LTD., Suzhou (CN)

(72) Inventors: Jingyu HU, Shanghai (CN); Xianlei FU, Shanghai (CN); Yanchao LIU, Shanghai (CN); Zhixiong ZHANG, Shanghai (CN); Weibo ZHANG, Shanghai (CN); Shaojing HU, Shanghai (CN); Kevin X. CHEN, Shanghai (CN)

(21) Appl. No.: 18/693,543

(22) PCT Filed: Sep. 23, 2022
(86) PCT No.: PCT/CN2022/121076

§ 371 Date: Mar. 20, 2024

(30) Foreign Application Priority Data
Sep. 23, 2021 (WO) PCT/CN2021/119861
Jun. 2, 2022 (WO) PCT/CN2022/096894

(51) Int. Cl. C07D 487/04; A61K 31/437; A61K 31/444; A61K 31/506; A61K 31/53; A61K 31/55; A61K 31/553; C07D 471/04; C07D 471/14; C07D 498/14

(52) U.S. Cl. CPC C07D 487/04; A61K 31/437; A61K 31/444; A61K 31/506; A61K 31/53; A61K 31/55; A61K 31/553; C07D 471/04; C07D 471/14; C07D 498/14

(57) ABSTRACT

Provide novel compounds of Formula (I) and pharmaceutical composition, pharmaceutically acceptable salt, prodrug, solvate, hydrate, tautomer, and isomer thereof, useful as FGFR inhibitors to modulate or inhibit the activity of FGFR enzymes. Further relates to, but is not limited to, methods for treating disorders associated with FGFR signaling with the compounds and compositions.

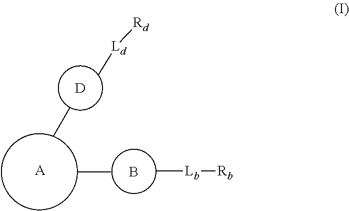

### TECHNICAL FIELD

This invention relates to certain novel fused heterocyclic derivatives compounds of Formula I, II, III, IV, V, VI, VII, I-I, or I-II, and a salt and pharmaceutical composition thereof, as FGFR inhibitors. Also described are used for treating a FGFR mediated disorders and cancers and methods of inhibiting FGFR activity, as well as methods for treating FGFR-associated disorders and cancers, and methods for preparing the compounds of Formula I, II, III, IV, V, VI, VII, I-I, or I-II. More particularly, this invention is directed to fused heterocyclic derivatives useful as inhibitors of FGFR, methods for producing such compounds and methods for treating a FGFR-mediated disorder.

### BACKGROUND

Fibroblast growth factor receptors (FGFR1, FGFR2, FGFR3, and FGFR4) are receptor tyrosine kinases consisting of an extracellular ligand binding domain and an intracellular tyrosine kinase domain. Binding of FGF ligands leads to receptor dimerization and a conformational change in the intracellular domain resulting in intermolecular transphosphorylation of the kinase domain and intracellular tail. Phosphorylated residues serve as docking sites for adaptor proteins that promote downstream signaling cascades leading to cellular behaviors including proliferation, survival, differentiation, migration, and angiogenesis. Deregulated FGFR signaling can occur via FGFR gene amplification or fusion, FGFR missense mutations, receptor overexpression resulting from dysregulation of epigenetic and/or transcriptional regulators, or upregulation of FGF ligands in the tumor microenvironment. FGFRs are expressed on many cell types; thus, aberrant FGFR signaling has been implicated in oncogenesis, tumor progression, and resistance to therapy across many tumor types. (For a review of FGFR signaling, see N. Turner and R. Grose, Nat. Rev. Cancer 2010, 10:116-129; and references cited therein.)

Pan-FGFR1-3 inhibitors have generated clinical responses in numerous FGFR-altered cancers, however on-target toxicity limits dosing of these inhibitors. One of the most common adverse effects of pan-FGFR inhibition is hyperphosphatemia. Regulation of phosphate reabsorption is mediated by FGFR1. Thus, there is a need for FGFR-selective inhibitors that spare FGFR1 (J. Gattineni et al., Am. J Physiol. Renal Physiol 2014, 306: F351-F358; X. Han et al., PLoS One 2016, 11:e0147845.). Cancers harboring FGFR2 gene fusions as well as those with FGFR2 amplification and/or FGFR2 activating mutations have demonstrated responses to pan-FGFR inhibition, however the low rates and duration of responses suggest they were limited by toxicities. Thus, there is a need for FGFR2-selective inhibitor compounds and methods for treating cancers and other disorders with these compounds. (For reviews of pan-FGFR1-3 inhibitors and clinical responses, see I S. Babina and N. C. Turner, Nat. Rev. Cancer 2017, 17:318-332; M. Katoh, Nat. Rev. Clin Oneal. 2019, 16:105-122; and references cited therein.)

### SUMMARY OF INVENTION

The present invention provides compounds of Formula (I), which are useful as inhibitors of FGFR2, including pharmaceutical compositions, pharmaceutically acceptable salt, prodrug, solvate, hydrate, tautomer, and isomer thereof.

The present invention provides the compounds represented by the Formula (I):

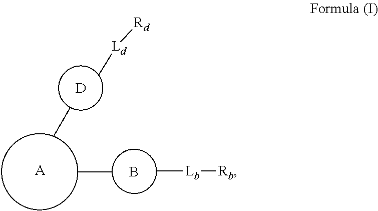

or a pharmaceutically acceptable salt, prodrug, solvate, hydrate, tautomer, and isomer thereof,
Ring A is selected from:

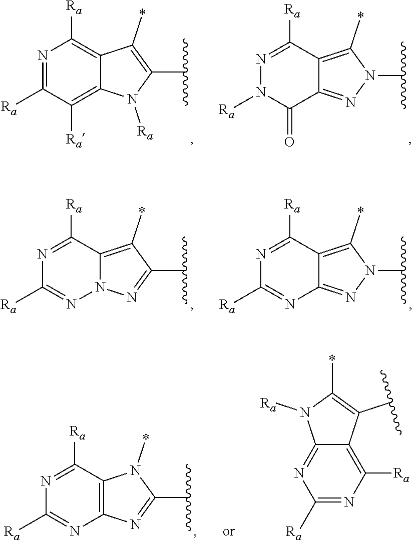

and “ [symbol] ” represents a bond to ring D and

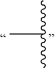

represents a bond to ring B;
Each of is Rₐ is independently optionally selected from H, D, CN, NH₂, OH, halogen, C₁₋₆alkyl, C₃₋₆ cycloalkyl, C₁₋₆alkoxyl, —(CH₂)₀₋₂NHR′, —(CH₂)₀₋₂N(R′)₂, or 3- to 6-membered saturated heterocyclic ring having 1 or 2 heteroatoms optionally independently selected from N, O, or S; and each of which is optionally independently unsubstituted or substituted with 1, 2, or 3 instances of D, OH, halogen, C₁₋₃alkyl, or C₁₋₃alkoxyl;
Ra′ is D, CN, OH, C₁₋₆alkoxyl, —CONH₂, —COR′, —CONHR′, or —CON(R′)₂; and each of which is optionally independently unsubstituted or substituted with 1, 2, or 3 instances of D, OH, halogen, C₁₋₃alkyl, or C₁₋₃alkoxyl;
Or Ra and Ra′ are taken together with their intervening atoms to form a 5- to 7-membered heterocyclic ring having at least one nitrogen atom, each of which is optionally independently unsubstituted or substituted with 1, 2, 3 instance of C₁₋₃alkoxy, C₁₋₃alkyl, D or halogen;
Ring B is phenylene; a bivalent saturated or partially unsaturated 3-10 membered carbocyclic ring; a bivalent saturated or partially unsaturated 3-10 membered heterocyclic ring having 1-5 heteroatoms independently selected from N, O, S, S(O), S(O)₂, S(O)(NH), or S(O)(NC₁₋₃alky); or a 5-10 membered heteroarylene having 1-5 heteroatoms or groups independently selected from N, O, or S; each of which is optionally independently unsubstituted or substituted with r instances of R_{B} in addition to -L_{b}-R_{b}; and r is 0, 1, 2, 3, or 4;
Ring D is phenylene; a bivalent saturated or partially unsaturated 3-10 membered carbocyclic ring; a bivalent saturated or partially unsaturated 3-10 membered heterocyclic ring having 1-5 heteroatoms independently selected from N, O, S, S(O), S(O)₂, S(O)(NH), or S(O)(NC₁₋₃alky); or a 5-10 membered heteroarylene having 1-5 heteroatoms independently selected from N, O, or S; and each of which is optionally independently unsubstituted or substituted with u instances of R_{D} in addition to -L_{d}-R_{d}; and u is 0, 1, 2, 3, or 4;
Each of R_{B} or R_{D} is independently optionally selected from -D, oxo, —OH, —NH₂, —CN, —NO₂, —(CH₂)₀₋₂ NHR′, —(CH₂)₀₋₂N(R′)₂, —(CH₂)₀₋₂OR′, R, —SO₂R, or —COR;
Each of L_{b} and L_{d} is independently optionally selected from a covalent bond, —O—, —S—, —NHS(O)₂—, —S(O)—, S(O)₂—, —CO—, —NH—, —(CH₂)₁₋₃, —CONH—, —NHCO—, —CONH(CH₂)₁₋₃—, —NHCO(CH₂)₁₋₃—, or —NHCONH—; and each of CH₂ is independently optionally unsubstituted or substituted with 1, 2, or 3 instances of D, halogen, C₁₋₃alkyl, or C₁₋₃haloalkyl; each of NH is independently optionally unsubstituted or substituted with C₁₋₃ alkyl;
R_{b} is halogen, CN,

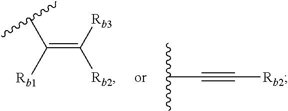

each of R_{b1}, R_{b2}, and R_{b3} is independently optionally selected from H, D, halogen, —CN, C₁₋₆ alkyl, C₃₋₆cycloalkyl, C₂₋₆alkenyl, C₂₋₆alknyl, —(CH₂)₀₋₂NHR′, —(CH₂)₀₋₂N(R′)₂, or —(CH₂)₀₋₂OR′; and each of which is optionally independently unsubstituted or substituted with 1, 2, 3 instances of halogen or D; or
R_{b1} and R_{b2}, R_{b2} and R_{b3}, R_{b1} and L_{b}, are taken with their intervening atoms to form 4-7 membered saturated or partially unsaturated ring having 0-2 heteroatoms or group selected from N, O, S, S(O), or S(O)₂;
R_{d} is H; D; C₁₋₆ alkyl; phenyl; a saturated or partially unsaturated 3-10 membered carbocyclic ring; a saturated or partially unsaturated 3-10 membered heterocyclic ring having 1-5 heteroatoms independently selected from N, O, S, S(O), or S(O)₂; or a 5-10 membered heteroaryl having 1-5 heteroatoms or groups independently selected from N, O, or S; each of which is optionally independently unsubstituted or substituted with 1, 2, 3 instances of R;
Each of R is optionally independently selected from halogen, —CN, —C₁₋₆ alkyl, —C₃₋₆ cycloalkyl —SC₁₋₆ alkyl, —N(C₁₋₆ alkyl)₂, —NHC₁₋₆ alkyl, —NHC₃₋₆ cycloalkyl, —C₁₋₆ alkoxy or —OC₃₋₆ cycloalkyl; and each of which is optionally unsubstituted or substituted with 1, 2, 3 instance of D or halogen;
Each of R′ is optionally independently selected from C₁₋₆alkyl or C₃₋₆cycloalkyl; or
two R′ are taken together with the nitrogen which both attached to form a 4- to 6-membered heterocyclic ring optionally having additional 1 or 2 heteroatoms or group selected from N, O, S, S(O), or S(O)₂.

In some embodiments, the present invention provides the compound of Formula (I) is represented by the Formula (II):

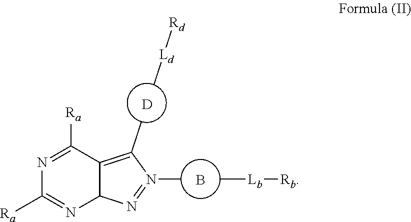

In some embodiments, the present invention provides the compound of Formula (I) is represented by the Formula (III):

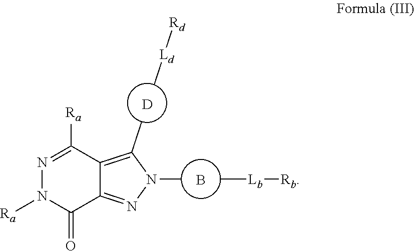

In some embodiments, the present invention provides the compound of Formula (I) is represented by the Formula (IV):

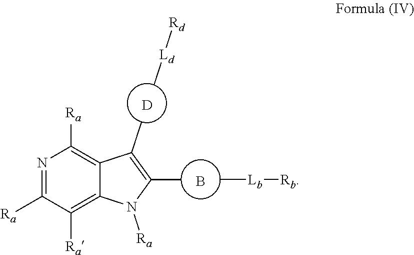

In some embodiments, the present invention provides the compound of Formula (I) is represented by the Formula (V):

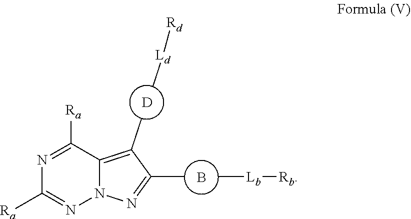

In some embodiments, the present invention provides the compound of Formula (I) is represented by the Formula (VI):

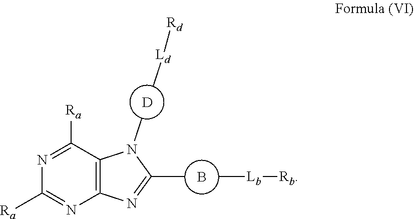

In some embodiments, the present invention provides the compound of Formula (I) is represented by the Formula (VII):

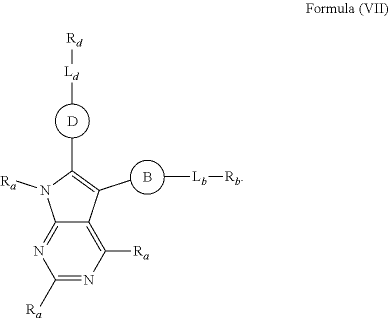

In some embodiments of Formula (I) provided by the present invention, each of Rₐ is independently optionally selected from H, D, CN, NH₂, OH, F, Cl, Br, C₁₋₃alkyl, C₁₋₃alkoxyl, cyclopropyl, cyclobutyl, cyclopentyl, cyclohexyl, —NHR′, —CH₂NHR′, —(CH₂)₂NHR′, —N(R′)₂, —CH₂N(R′)₂, —(CH₂)₂N(R′)₂, 3-membered saturated heterocyclic ring, 4-membered saturated heterocyclic ring, 5-membered saturated heterocyclic ring, or 6-membered saturated heterocyclic ring; furthermore each of heterocyclic ring has 1 or 2 heteroatoms optionally independently selected from N, O, or S; and each of which is optionally independently unsubstituted or substituted with 1, 2, or 3 instances of D, OH, F, Cl, Br, methyl, ethyl, propyl, methoxy, or ethoxy.

In some embodiments of Formula (I) provided by the present invention, each of Rₐ is independently optionally selected from H, D, CN, NH₂, OH, F, Cl, methyl, ethyl, propyl, iso-propyl, methoxy, ethoxy, propoxy, cyclopropyl, cyclobutyl, cyclopentyl, cyclohexyl, —NHR′, —CH₂NHR′, —(CH₂)₂NHR′, —N(R′)₂, —CH₂N(R′)₂, —(CH₂)₂N(R′)₂, 4-membered saturated heterocyclic ring, 5-membered saturated heterocyclic ring, or 6-membered saturated heterocyclic ring; furthermore each of saturated heterocyclic ring has 1 or 2 heteroatoms optionally independently selected from N or O; and each of which is optionally independently unsubstituted or substituted with 1, 2, or 3 instances of D, OH, F, Cl, methyl, ethyl, methoxy, or ethoxy.

In some embodiments of Formula (I) provided by the present invention, Rₐ is independently optionally selected from H, D, CN, NH₂, OH, F, methyl, cyclopropyl, cyclobutyl, —NHR′, —CH₂NHR′, —(CH₂)₂NHR′, —N(R′)₂, —CH₂N(R′)₂, —(CH₂)₂N(R′)₂, 4-membered saturated heterocyclic ring, 5-membered saturated heterocyclic ring, or 6-membered saturated heterocyclic ring; furthermore each of heterocyclic ring has 1 or 2 heteroatoms optionally independently selected from N, or O; and each of which is optionally independently unsubstituted or substituted with 1, 2, or 3 instances of D, OH, or F.

In some embodiments of Formula (I) provided by the present invention, each of Ra is

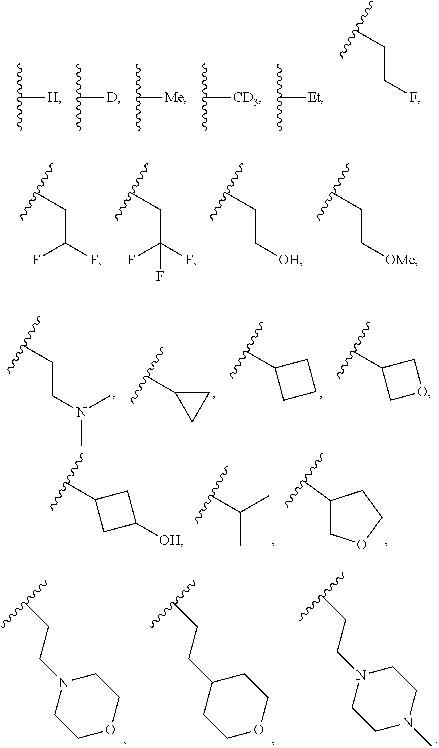

In some embodiments of Formula (I) provided by the present invention, Ra′ is D, CN, OH, C₁₋₃alkoxyl, —CONH₂, —COR′, —CONHR′, or —CON(R′)₂; and each of which is optionally independently unsubstituted or substituted with 1, 2, or 3 instances of D, OH, F, Cl, Br, methyl, ethyl, propyl, iso-propyl, methoxy, ethoxy, or propoxy.

In some embodiments of Formula (I) provided by the present invention, Ra′ is D, CN, OH, methoxy, ethoxy, propoxy, —CONH₂, —COR′, —CONHR′, or —CON(R′)₂; and each of which is optionally independently unsubstituted or substituted with 1, 2, or 3 instances of D, OH, F, Cl, methyl, ethyl, methoxy, or ethoxy.

In some embodiments of Formula (I) provided by the present invention, Ra′ is D, CN, or —CONH₂.

In some embodiments of Formula (I) provided by the present invention, Ra and Ra′ are taken together with their intervening atoms to form a 5-membered heterocyclic ring, 6-membered heterocyclic ring, or 7-membered heterocyclic ring, furthermore each of heterocyclic ring also contains optionally one additional heteroatom selected from N or S; and each of which is optionally independently unsubstituted or substituted with 1, 2, 3 instance of methyl, ethyl, methoxy, ethoxy, D, F, or Cl.

In some embodiments of Formula (I) provided by the present invention, Ra and Ra′ are taken together with their intervening atoms to form a 6-membered heterocyclic ring having one N atom or 6-membered heterocyclic ring having one N atom and one O atom; and each of which is optionally independently unsubstituted or substituted with 1, 2, 3 instance of methyl, D, F, or Cl.

In some embodiments of Formula (I) provided by the present invention, B is phenylene; a bivalent saturated or partially unsaturated 5- to 9-membered carbocyclic ring; a bivalent saturated or partially unsaturated 5- to 9-membered heterocyclic ring having 1-4 heteroatoms independently selected from N, O, S, S(O), or S(O)₂; or a 5-9 membered heteroarylene having 1-4 heteroatoms independently selected from N, O, or S; each of which is optionally independently unsubstituted or substituted with r instances of R_{B} in addition to -L_{b}-R_{b}.

In some embodiments of Formula (I) provided by the present invention, ring B is phenylene; a bivalent saturated or partially unsaturated 5-, 6-, 7-, 8- or 9-membered carbocyclic ring; a bivalent saturated or partially unsaturated 5-, 6-, 7-, 8-, or 9-membered heterocyclic ring having 1, 2, 3, or 4 heteroatoms independently selected from N, O, or S; or a 5-, 6-, 7- or 8-, or 9-membered heteroarylene having 1, 2, 3, or 4 heteroatoms independently selected from N, O, or S; each of which is optionally independently unsubstituted or substituted with r instances of R_{B} in addition to -L_{b}-R_{b}.

In some embodiments of Formula (I) provided by the present invention, ring B is phenylene; a bivalent saturated 5-, 6-, or 7-membered heterocyclic ring having 1, 2, or 3 heteroatoms independently selected from N or O; or a 5-, 6-, or 7-membered heteroarylene having 1, 2, or 31 or 2 heteroatoms independently selected from N or O; each of which is optionally independently unsubstituted or substituted with r instances of R_{B} in addition to -L_{b}-R_{b}.

In some embodiments of Formula (I) provided by the present invention, ring B is phenylene; a bivalent saturated 5- or 6-membered heterocyclic ring having 1 or 2 heteroatoms independently selected from N; or a 5- or 6-membered heteroarylene having 1 or 2 heteroatoms independently selected from N; each of which is optionally independently unsubstituted or substituted with r instances of R_{B} in addition to -L_{b}-R_{b}.

In some embodiments of Formula (I) provided by the present invention, ring B is phenylene,

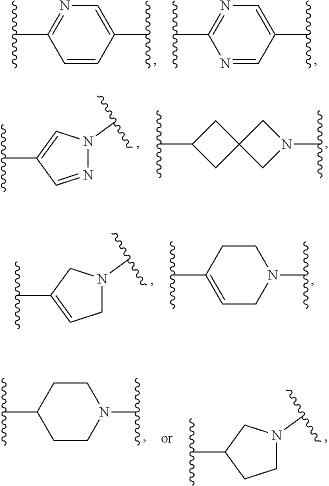

and each of

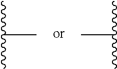

can optionally attach to ring A or -L_{b}-R_{b}; each of which is optionally independently unsubstituted or substituted with r instances of R_{B} in addition to -L_{b}-R_{b}.

In some embodiments of Formula (I) provided by the present invention, ring D is phenylene; a bivalent saturated or partially unsaturated 5- to 9-membered carbocyclic ring; a bivalent saturated or partially unsaturated 5- to 9-membered heterocyclic ring having 1-4 heteroatoms independently selected from N, O, S, S(O), or S(O)₂; or a 5-9 membered heteroarylene having 1-4 heteroatoms independently selected from N, O, or S; and each of which is optionally independently unsubstituted or substituted with u instances of R_{D} in addition to -L_{d}-R_{d}.

In some embodiments of Formula (I) provided by the present invention, ring D is phenylene; a bivalent saturated or partially unsaturated 5-, 6-, 7-, 8- or 9-membered carbocyclic ring; a bivalent saturated or partially unsaturated 5-, 6-, 7-, 8-, or 9-membered heterocyclic ring having 1, 2, 3, or 4 heteroatoms independently selected from N, O, or S; or a 5-, 6-, 7- or 8-, or 9-membered heteroarylene having 1, 2, 3, or 4 heteroatoms independently selected from N, O, or S; and each of which is optionally independently unsubstituted or substituted with u instances of R_{D} in addition to -L_{d}-R_{d}.

In some embodiments of Formula (I) provided by the present invention, ring D is phenylene; a bivalent saturated 5-, 6-, or 7-membered heterocyclic ring having 1, 2, or 3 heteroatoms independently selected from N or O; or a 5-, 6-, or 7-membered heteroarylene having 1, 2, or 31 or 2 heteroatoms independently selected from N or O; and each of which is optionally independently unsubstituted or substituted with u instances of R_{D} in addition to -L_{d}-R_{d}.

In some embodiments of Formula (I) provided by the present invention, ring D is phenylene; a bivalent saturated 5- or 6-membered heterocyclic ring having 1 or 2 heteroatoms independently selected from N; or a 5- or 6-membered heteroarylene having 1 or 2 heteroatoms independently selected from N; and each of which is optionally independently unsubstituted or substituted with u instances of R_{D} in addition to -L_{d}-R_{d} In some embodiments of Formula (I) provided by the present invention, each of ring D is a phenylene;

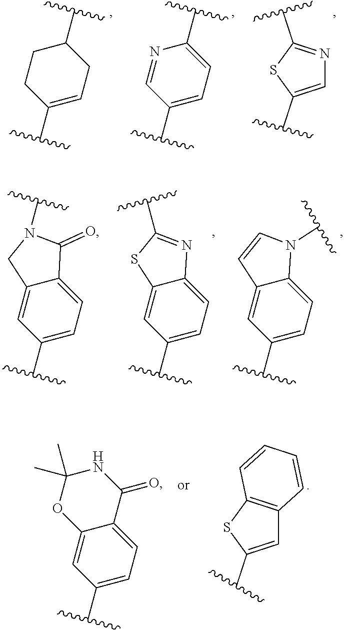

In some embodiments of Formula (I) provided by the present invention, each of RB or RD is independently optionally selected from —D, oxo, —OH, —NH₂, —CN, —NO₂, —NHR′, —CH2NHR′, —(CH₂)₂NHR′, —N(R′)₂, —CH₂N(R′)₂, —(CH₂)₂N(R′)₂, —OR′, —CH₂OR′, —(CH₂)₂OR′, R, ₂SO₂R, or —COR.

In some embodiments of Formula (I) provided by the present invention, each of RB or RD is independently optionally selected from —D, oxo, —OH, —NH₂, —CN, —NO₂, R, —NHR′, —CH₂NHR′, —(CH₂)₂NHR′, —N(R′)₂, —CH₂N(R′)₂, or —(CH₂)₂N(R′)₂.

In some embodiments of Formula (I) provided by the present invention, each of RB is independently

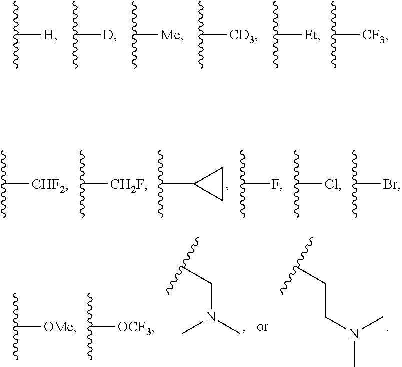

In some embodiments of Formula (I) provided by the present invention, each of R_{D} is independently selected from H, D, F, Cl, CN, OH, methyl, ethyl, propyl, cyclopropyl, -SMe, -OMe, -OEt, —CFH₂, —CF₂H, —CF₃; each of which is independently optionally unsubstituted or substituted with 1, 2, or 3 instances of F, Cl, or D.

In some embodiments of Formula (I) provided by the present invention, each of L_{b} and La is independently optionally selected from a covalent bond, —O—, —S—, —CO—, —NH—, —CH₂—, —(CH₂)₂, —CONH—, —NHCO—, —CONHCH₂—, —CONH(CH₂)₂—, —NHCO(CH₂)₂—, —NHCOCH₂—, or —NHCONH—; and each of which is independently optionally unsubstituted or substituted with w instances of R_{L}.

In some embodiments of Formula (I) provided by the present invention, each of L_{b} and L_{d} is independently optionally selected from a covalent bond, —O—, —S—, —CO—, —NH—, —CONH—, —NHCO—; and each of which is independently optionally unsubstituted or substituted with 1, 2, or 3 instances of D, F, Cl, Br, methyl, ethyl, propyl, methoxy, ethoxy, propoxy, methyl substituted with F or Cl, ethyl substituted with F or Cl, propyl substituted with F or Cl, methoxy substituted with F or Cl, ethoxy substituted with F or Cl, or propoxy substituted with F or Cl.

In some embodiments of Formula (I) provided by the present invention, each of L_{b} and L_{d} is independently optionally selected from a covalent bond, —O—, —S—, —CO—, —NH—, —CONH—, —NHCO—; and each of which is independently optionally unsubstituted or substituted with 1, 2, or 3 instances of D, F, Cl, methyl, methoxy, methyl substituted with F or Cl, or methoxy substituted with F or Cl.

In some embodiments of Formula (I) provided by the present invention, R_{b} is halogen, CN,

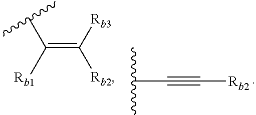

In some embodiments of Formula (I) provided by the present invention, R_{b} is

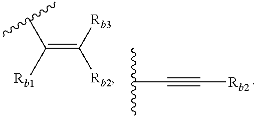

In some embodiments of Formula (I) provided by the present invention, each of R_{b1}, R_{b2}, and R_{b3} is independently optionally selected from H, D, F, Cl, Br, —CN, C₁₋₃ alkyl, C₃₋₆cycloalkyl, C₂₋₃alkenyl, C₂₋₆alknyl, —CH₂NHR′, —(CH₂)₂NHR′, —CH₂N(R′)₂, —(CH₂)₂N(R′)₂, —OCR′—CH₂OCR′, or —(CH₂)₂OCR′; each of which is optionally independently unsubstituted or substituted with 1, 2, 3 instances of F, Cl, Br, or D.

In some embodiments of Formula (I) provided by the present invention, each of R_{b1}, R_{b2}, and R_{b3} is independently optionally selected from H, D, F, Cl, —CN, methyl, ethyl, propyl, i-propylvinyl, ethynyl, or ethynyl; each of which is optionally independently unsubstituted or substituted with 1, 2, 3 instances of F, Cl, or D.

In some embodiments of Formula (I) provided by the present invention, each of R_{b1}, R_{b2}, and R_{b3} is independently optionally selected from H, D, F, Cl, —CN, or methyl; each of which is optionally independently unsubstituted or substituted with 1, 2, 3 instances of F or D.

In some embodiments of Formula (I) provided by the present invention, R_{d} is H; D; C₁₋₃ alkyl; phenyl; a 3-, 4-, 5-, 6-, or 7-membered saturated or partially unsaturated monocyclic carbocyclic ring; a 5-, 6-, 7-, 8-, or 9-membered saturated or partially unsaturated bicyclic carbocyclic ring; a 5- or 6-membered monocyclic heteroaryl ring having 1 or 2 heteroatoms independently selected from N, O or S; a 3-, 4-, 5-, 6-, or 7-membered saturated or partially unsaturated monocyclic heterocyclic ring having 1 or 2 heteroatoms independently selected from N, O, or S; or a 5-, 6-, 7-, 8-, or 9-membered saturated or partially unsaturated bicyclic heterocyclic ring having 1 or 2 heteroatoms independently selected from N, O, or S; each of which is optionally independently unsubstituted or substituted with 1, 2, 3 instances of R.

In some embodiments of Formula (I) provided by the present invention, R_{d} is H; D; methyl; ethyl; propyl; i-propyl; phenyl; 5- or 6-membered monocyclic heteroaryl ring having 1 or 2 heteroatoms independently selected from N or S; a 3-, 4-, 5-, 6-, or 7-membered saturated monocyclic carbocyclic ring; a 4-, 5-, or 6-membered saturated monocyclic heterocyclic ring having 1 or 2 heteroatoms independently selected from N, O, or S; or a 5-, 6-, or 7-membered saturated bicyclic carbocyclic ring; each of which is optionally independently unsubstituted or substituted with 1, 2, 3 instances of R.

In some embodiments of Formula (I) provided by the present invention, R_{d} is H; D; methyl; ethyl; phenyl; 5- or 6-membered monocyclic heteroaryl ring having 1 or 2 heteroatoms independently selected from N or S; a 3-, 4-, 5-, or 6-membered saturated carbocyclic ring; a 4-, 5-, or 6-membered saturated monocyclic heterocyclic ring having 1 or 2 heteroatoms independently selected from N; or a 5-, 6-, or 7-membered saturated bicyclic carbocyclic ring; each of which is optionally independently unsubstituted or substituted with 1, 2, 3 instances of R.

In some embodiments of Formula (I) provided by the present invention, R_{d} is

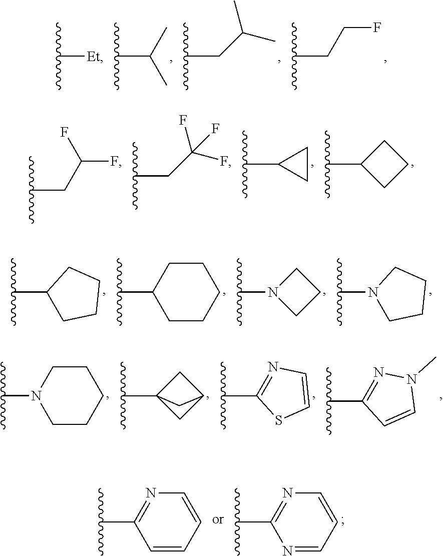

each of which is optionally independently unsubstituted or substituted with 1, 2, 3 instances of R.

In some embodiments of Formula (I) provided by the present invention, R is optionally independently selected from F, Cl, Br, C₁₋₃ alkyl, cyclopropyl, cyclobutyl, cyclopentyl, cyclohexyl, —SC₁₋₃alkyl, —N(C₁₋₃ alkyl)₂, —NHC₁₋₃alkyl, —NH-cyclopropyl, —NH-cyclobutyl, —NH-cyclopentyl, —NH-cyclohexyl, —C₁₋₃ alkoxy, —O-cyclopropyl, —O-cyclobutyl, —O— cyclopentyl, or —O-cyclohexyl; and each of which is optionally unsubstituted or substituted with 1, 2, 3 instance of D, F, Cl, or Br.

In some embodiments of Formula (I) provided by the present invention, R is optionally independently selected from F, Cl, Br, methyl, ethyl, propyl, i-propyl, methoxy, ethoxy, cyclopropyl, cyclobutyl, cyclopentyl, —NH-cyclobutyl, —C₁₋₃ alkoxy, —O-cyclopropyl or —O-cyclobutyl; and each of which is optionally unsubstituted or substituted with 1, 2, 3 instance of D, F, or Cl.

In some embodiments of Formula (I) provided by the present invention, R is optionally independently selected from F, Cl, methyl, ethyl, methoxy, or cyclopropyl; and each of which is optionally unsubstituted or substituted with 1, 2, 3 instance of D or F.

In some embodiments of Formula (I) provided by the present invention, each of R′ is optionally independently selected from C₁₋₃alkyl, cyclopropyl, cyclobutyl, or cyclopentyl.

In some embodiments of Formula (I) provided by the present invention, each of R′ is optionally independently selected from methyl, ethyl, propyl, i-propyl, cyclopropyl, cyclobutyl, or cyclopentyl.

In some embodiments, the present invention furthermore provides the compound of Formula (I) is represented by the Formula (I-I) or Formula (I-II):

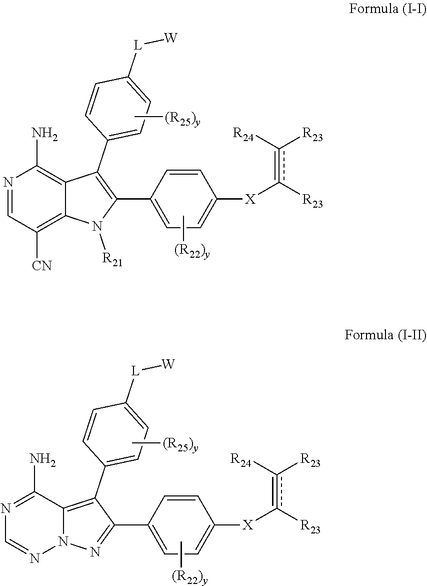

R₂₁ is selected from H, D, CN, C₁₋₃alkyl, C₁₋₃alkoxyl, C₃₋₆cycloalkyl, —(CH₂)₀₋₂—NHC₁₋₃alkyl, —(CH₂)₀₋₂N(C₁₋₃alkyl)₂, or 3- to 6-membered saturated heterocyclic ring having 1 or 2 heteroatoms optionally independently selected from N, O, or S; and each of which is optionally independently unsubstituted or substituted with 1, 2, or 3 instances of D, OH, halogen, C₁₋₃alkyl, C₁₋₃alkoxyl, or C₃₋₆cycloalkyl;
Each of R₂₂ is optionally independently selected from H, D, halogen, CN, NH₂, OH, C₁₋₃alkyl, C₁₋₃alkoxyl, C₃₋₆cycloalkyl, —(CH₂)₀₋₂—NHC₁₋₃alkyl, or —(CH₂)₀₋₂N(C₁₋₃alkyl)₂, and each of which is optionally independently unsubstituted or substituted with 1, 2, or 3 instances of R_{z}; or
R₂₁ and R₂₂ are taken together with their intervening atoms to form a 5- to 7-membered heterocyclic ring having at least one nitrogen atom;
Each of R₂₅ is optionally independently H, D, halogen, CN, OH, R″, N(R″)₂, OR″, or —SR″;
y is 0, 1, 2, 3, or 4;
Each of X or L is optionally independently selected from a covalent bond, —O—, —S—, —S(O)—, —NHS(O)₂—, —S(O)₂—, —CO—, —NH—, —CH₂—, —CONH—, —NHCO—, —CONHCH₂—, —NHCONH—, —NHCOCH₂—, or —NHCOCH₂CH₂—; and each of CH₂ is independently optionally unsubstituted or substituted with 1, 2, or 3 instances of D, halogen, C₁₋₃alkyl, or C₁₋₃haloalkyl; each of NH is independently optionally unsubstituted or substituted with C₁₋₃alkyl;
[symbol] is a bivalent bond or a trivalent bond;
1) when [symbol] is a trivalent bond, each of R₂₃ is absent, and each of R₂₄ is independently optionally selected from H, D, —C₀₋₃ alkylene-N(R″)₂, —C₀₋₃ alkylene-OR″, or R″; each of which is optionally unsubstituted or substituted with 1, 2, or 3 instances of D or halogen;
2) when [symbol] is a bivalent bond, each of R₂₃ or R₂₄ is independently optionally selected from H, D, halogen, —C₀₋₃alkylene-N(R″)₂, —C₀₋₃ alkylene-OR″, or R″; each of which is optionally unsubstituted or substituted with 1, 2, or 3 instances of D or halogen;
Each of W is optionally independently selected from C₁₋₃alkyl, phenyl, 4- to 6-membered saturated or partially saturated heterocyclic ring having 1 or 2 heteroatoms selected from N, O, or S, 5- or 6-membered heteroaryl having 1 to 3 heteroatoms selected from N, O, or S, or 3- to 6-membered saturated or partially saturated carbocyclic ring; each of which is optionally independently unsubstituted or substituted with 1, 2, or 3 instances of R_{w};
Each of R_{w} is optionally independently selected from D, halogen, OH, CN, NH₂, C₁₋₃alkyl, C₁₋₃ haloalkyl, C₃₋₆ cycloalkyl, C₂₋₃alkenyl or C₂₋₃alknyl; and each of which is optionally independently unsubstituted or substituted with 1, 2, or 3 instances of R_{z};
Each of R_{z} is optionally independently selected from H, D, halogen, CN, —N(R″)₂, —OR″, or R″.

Each of R″ is optionally independently C₁₋₃ alkyl, or C₃₋₆ cycloalkyl, each of which is optionally unsubstituted or substituted with 1, 2, or 3 instances of halogen or D;
In some embodiments of Formula (I) provided by the present invention, R₂₁ is selected from H, D, CN, NH₂, OH, methyl, ethyl, propyl, i-propyl, methoxy, ethoxy, cyclopropyl, cyclobutyl, cyclopentyl, cyclohexyl, —(CH₂)₀₋₂—NHCH₃, —(CH₂)₀₋₂—NHCH₂CH₃, —(CH₂)₀₋₂N(CH₃)₂, —(CH₂)₀₋₂ N(CH₂CH₃)₂, —(CH₂)₀₋₂N(CH₃)(CH₂CH₃), 4-membered saturated heterocyclic ring, 5-membered saturated heterocyclic ring, or 6-membered saturated heterocyclic ring having one or two heteroatom selected from N or O; and each of which is optionally independently unsubstituted or substituted with 1, 2, or 3 instances of D, OH, F, Cl, Br, methoxy, ethoxy, or propoxy; the said R₂₂ is optionally independently selected from H, D, F, Cl, Br, CN, NH₂, OH, methyl, ethyl, propyl, i-propyl, methoxy, ethoxy, cyclopropyl, cyclobutyl, cyclopentyl, cyclohexyl, —(CH₂)₀₋₂—NHCH₃, —(CH₂)₀₋₂—NHCH₂CH₃, —(CH₂)₀₋₂N(CH₃)₂, —(CH₂)₀₋₂N(CH₂CH₃)₂, or —(CH₂)₀₋₂N(CH₃CH₂CH₃); and each of which is optionally independently unsubstituted or substituted with 1, 2, or 3 instances of R_{z}.

In some embodiments of Formula (I) provided by the present invention, R₂₁ is selected from H, D, CN, NH₂, OH, methyl, ethyl, methoxy, cyclopropyl, cyclohexyl, 4-membered saturated heterocyclic ring, or 5-membered saturated heterocyclic ring having one or two heteroatom selected from N or O; and each of which is optionally independently unsubstituted or substituted with 1, 2, or 3 instances of D, OH, F, or C₁; and each of R₂₂ is selected from H, D, F, Cl, CN, NH₂, OH, methyl, ethyl, methoxy, ethoxy, cyclopropyl, cyclobutyl, —NHCH₃, —CH₂NHCH₃, —CH₂CH₂NHCH₃, —CH₂NHCH₂CH₃, —N(CH₃)₂, —CH₂N(CH₃)₂, —(CH₂)₂N(CH₃)₂, —N(CH₃CH₂CH₃), —CH₂N(CH₃CH₂CH₃), or —(CH₂)₂N(CH₃CH₂CH₃); and each of which is optionally independently unsubstituted or substituted with 1, 2, or 3 instances of R_{z}.

In some embodiments of Formula (I) provided by the present invention, R₂₁ is selected from

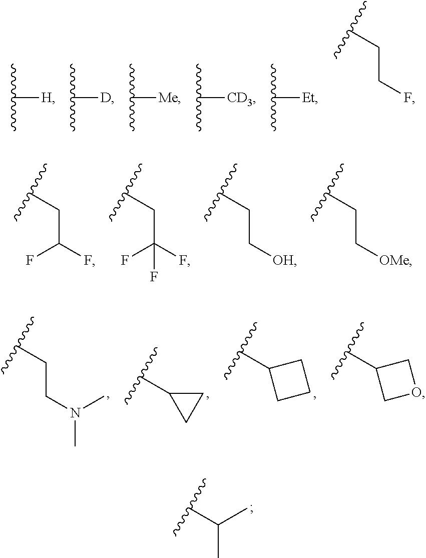

the said R₂₂ is

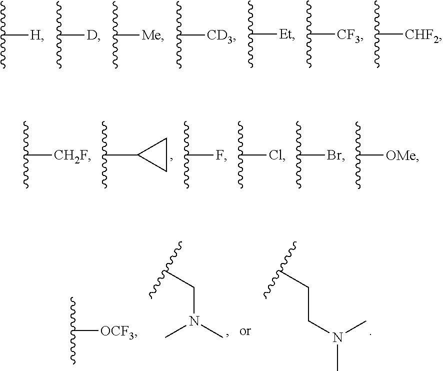

In some embodiments of Formula (I) provided by the present invention, R₂₁ is selected from

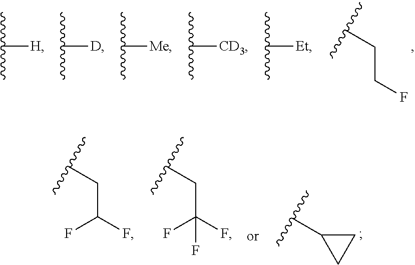

the said R₂₂ is

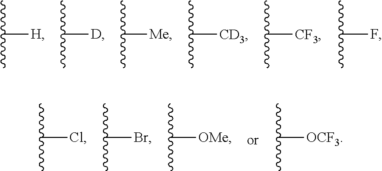

In some embodiments of Formula (I) provided by the present invention, R₂₁ and R₂₂ are taken together with their intervening atoms to form a 5-membered heterocyclic ring, 6-membered heterocyclic ring, 7-membered heterocyclic ring, 5-membered carbocyclic ring, 6-membered carbocyclic ring, or 7-membered carbocyclic ring.

In some embodiments of Formula (I) provided by the present invention, heterocyclic ring additionally have also one heteroatom selected from oxygen atom, nitrogen atom, or sulfur atom.

In some embodiments of Formula (I) provided by the present invention, heterocyclic ring is 6-membered heterocyclic ring additionally having one oxygen atom, and the said carbocyclic ring is 6-membered carbocyclic ring.

In some embodiments of Formula (I) provided by the present invention, each of R₂₅ is optionally independently H, D, F, Cl, Br, CN, NH₂, OH, methyl, ethyl, propyl, i-propyl, —S— methyl, —S-ethyl, —S-propyl, methoxy, ethoxy, or propoxy; each of which is optionally unsubstituted or substituted with 1, 2, 3 instances of D, F, Cl, or Br.

In some embodiments of Formula (I) provided by the present invention, each of R₂₅ is optionally independently H, D, F, Cl, methyl, ethyl, —S-methyl, —S-ethyl, methoxy, or ethoxy; each of which is optionally unsubstituted or substituted with 1, 2, 3 instances of D or F.

In some embodiments of Formula (I) provided by the present invention, each of R₂₅ is optionally independently H, D, F, Cl, methyl, ethyl, —S-methyl, methoxy, or ethoxy; each of which is optionally unsubstituted or substituted with 1, 2, 3 instances of F.

In some embodiments of Formula (I) provided by the present invention, each of X or L is optionally independently selected from a covalent bond, —O—, —CO—, —NH—, —CH₂—, —CONH—, or —NHCO—.

In some embodiments of Formula (I) provided by the present invention, each of X is optionally independently selected from —NH—; the said each of L is optionally independently selected from —O—, —NH—, —CONH—, or —NHCO—.

In some embodiments of Formula (I) provided by the present invention, [symbol] is a bivalent bond and the said each of R₂₃ or R₂₄ is independently optionally selected from H, D, F, Cl, Br, —N(R″)₂, —CH₂N(R″)₂, —(CH₂)₂N(R″)₂, —(CH₂)₃N(R″)₂, or R″; each of which is optionally unsubstituted or substituted with 1, 2, or 3 instances of D, F, Cl, or Br.

In some embodiments of Formula (I) provided by the present invention, [symbol] is a bivalent bond and the said each of R₂₃ or R₂₄ is independently optionally selected from H, D, F, Cl, methyl, ethyl, propyl, i-propy, or cyclopropyl; each of which is optionally unsubstituted or substituted with 1, 2, or 3 instances of D, F or Cl.

In some embodiments of Formula (I) provided by the present invention, [symbol] is a bivalent bond and the said each of R₂₃ or R₂₄ is independently optionally selected from H, D, F, or methyl, and the methyl is optionally unsubstituted or substituted with 1, 2, or 3 instances of D or F.

In some embodiments of Formula (I) provided by the present invention, [symbol] is a trivalent bond and the said each of R₂₃ is absent, and the said each of R₂₄ is independently optionally selected from H, D, methyl, ethyl, propyl, iso-propyl, or cyclopropyl; each of which is optionally unsubstituted or substituted with 1, 2, or 3 instances of D, F, or Cl.

In some embodiments of Formula (I) provided by the present invention, [symbol] is a trivalent bond and the said each of R₂₃ is absent, and the said each of R₂₄ is independently optionally selected from H, D, or methyl; the said methyl is optionally unsubstituted or substituted with 1, 2, or 3 instances of D or F

In some embodiments of Formula (I) provided by the present invention, W is optionally independently selected from methyl; phenyl; 4-, 5-, or 6-membered saturated heterocyclic ring having 1 or 2 heteroatoms selected from N, O, or S; 5- or 6-membered heteroaryl having 1 or 2 heteroatoms selected from N or S; or 3-, 4-, 5-, or 6-membered saturated carbocyclic ring.

In some embodiments of Formula (I) provided by the present invention, W is optionally independently selected from 5- or 6-membered saturated heterocyclic ring having 1 nitrogen atom, 5-membered heteroaryl having 1 nitrogen atom and optionally additionally 1 sulfur atom, 6-membered heteroaryl having 1 or 2 nitrogen atom, 3-membered saturated carbocyclic ring, 4-membered saturated carbocyclic ring, 5-membered saturated carbocyclic ring, or 6-membered saturated carbocyclic ring;

In some embodiments of Formula (I) provided by the present invention, each of R_{w} is optionally independently selected from D, F, Cl, Br, OH, OMe, CN, NH₂, methyl, ethyl, propyl, i-propyl, methyl substituted with F or Cl, ethyl substituted with F or Cl, propyl substituted with F or Cl, i-propyl substituted with F or Cl, cyclopropyl, cyclobutyl, cyclopentyl, viny or ethynyl; and each of which is optionally independently unsubstituted or substituted with 1, 2, or 3 instances of R.

In some embodiments of Formula (I) provided by the present invention, each of R_{w} is optionally independently selected from D; F; Cl; methyl; methyl substituted with 1, 2, or 3 instance of F or D; methoxy; or cyclopropyl.

In some embodiments of Formula (I) provided by the present invention, each of R is optionally independently selected from D, F, Cl, Br, CN, —N(R″)₂, —OR″, or R″.

In some embodiments of Formula (I) provided by the present invention, each of R is optionally independently selected from D, F, Cl, Br, CN, NH₂, methyl, ethyl, propyl, or i-propyl.

In some embodiments of Formula (I) provided by the present invention, each of R is optionally independently selected from D, F, Cl, or methyl.

In some embodiments of Formula (I) provided by the present invention, each of R″ is optionally independently methyl, ethyl, propyl, i-propyl, cyclopropyl, cyclobutyl, or cyclopentyl; each of which is optionally unsubstituted or substituted with 1, 2, or 3 instances of F, Cl, or D.

In some embodiments of Formula (I) provided by the present invention, each of R″ is optionally independently methyl, ethyl, or cyclopropyl; each of which is optionally unsubstituted or substituted with 1, 2, or 3 instances of F or D.

In some embodiments of Formula (I) provided by the present invention, the compound is independently selected from:
N-(4-(3-(3-methoxy-4-((4-methylpyrimidin-2-yl)oxy)phenyl)imidazo[1,2-b]pyridazin-2-yl)phenyl)acrylamide;
N-(4-(9-(3-methoxy-4-((4-methylpyrimidin-2-yl)oxy)phenyl)-9H-purin-8-yl)phenyl) acrylamide;
N-(4-(7-(3-methoxy-4-((4-methylpyrimidin-2-yl)oxy)phenyl)imidazo[1,2-b][1,2,4]triazin-6-yl)phenyl)acrylamide;
N-(4-(4-amino-3-(3-methoxy-4-((4-methylpyrimidin-2-yl)oxy)phenyl)-1H-pyrazolo[3,4-d]pyrimidin-1-yl)phenyl)acrylamide;
N-(4-(5-amino-3-(3-methoxy-4-((4-methylpyrimidin-2-yl)oxy)phenyl)imidazo[1,2-a]pyrimidin-2-yl)phenyl)acrylamide;
N-(4-(4-amino-5-cyano-3-(3-methoxy-4-((4-methylpyrimidin-2-yl)oxy)phenyl)-1-methyl-1H-pyrrolo[2,3-b]pyridin-2-yl)phenyl)acrylamide;
N-(4-(4-amino-7-cyano-3-(3-methoxy-4-((4-methylpyrimidin-2-yl)oxy)phenyl)-1-methyl-1H-pyrrolo[3,2-c]pyridin-2-yl)phenyl)acrylamide;
N-(4-(4-amino-5-(3-methoxy-4-((4-methylpyrimidin-2-yl)oxy)phenyl)-2,7-dimethyl-7H-pyrrolo[2,3-d]pyrimidin-6-yl)phenyl)acrylamide;
N-(4-(4-amino-2-chloro-5-(3-methoxy-4-((4-methylpyrimidin-2-yl)oxy)phenyl)-7-methyl-7H-pyrrolo[2,3-d]pyrimidin-6-yl)phenyl)acrylamide;
N-(4-(4-methoxy-5-(3-methoxy-4-((4-methylpyrimidin-2-yl)oxy)phenyl)-7-methyl-7H-pyrrolo[2,3-d]pyrimidin-6-yl)phenyl)acrylamide;
N-(4-(4-cyclopropyl-5-(3-methoxy-4-((4-methylpyrimidin-2-yl)oxy)phenyl)-7-methyl-7H-pyrrolo[2,3-d]pyrimidin-6-yl)phenyl)acrylamide;
N-(4-(4-(difluoromethoxy)-5-(3-methoxy-4-((4-methylpyrimidin-2-yl)oxy)phenyl)-7-methyl-7H-pyrrolo[2,3-d]pyrimidin-6-yl)phenyl)acrylamide;
N-(4-(4-cyano-5-(3-methoxy-4-((4-methylpyrimidin-2-yl)oxy)phenyl)-7-methyl-7H-pyrrolo[2,3-d]pyrimidin-6-yl)phenyl)acrylamide;
N-(4-(4-amino-6-(3-methoxy-4-((4-methylpyrimidin-2-yl)oxy)phenyl)-7-methyl-7H-pyrrolo[2,3-d]pyrimidin-5-yl)phenyl)acrylamide;
N-(4-(4-amino-5-(4-(pyrrolidine-1-carbonyl)phenyl)pyrazolo[5,1-f][1,2,4]triazin-6-yl)-3-fluorophenyl)methacrylamide;
N-(4-(4-amino-5-(3-methoxy-4-((5-(methylamino)pyrimidin-2-yl)oxy)phenyl)pyrazolo[5,1-f][1,2,4]triazin-6-yl)phenyl)acrylamide;
N-(4-(4-amino-5-(3-methoxy-4-(pyrrolidine-1-carbonyl)phenyl)pyrazolo[5,1-f][1,2,4]triazin-6-yl)phenyl)acrylamide;
N-(4-(4-amino-5-(3-methoxy-4-((1-methyl-1H-pyrazol-3-yl)oxy)phenyl)pyrazolo[5,1-f][1,2,4]triazin-6-yl)phenyl)acrylamide;
N-(4-(4-amino-5-(4-(pyrimidin-2-yloxy)phenyl)pyrazolo[5,1-f][1,2,4]triazin-6-yl)phenyl) acrylamide;
2-(4-acrylamidophenyl)-4-amino-3-(3-methoxy-4-((4-methylpyrimidin-2-yl)oxy)phenyl)-1-methyl-1H-pyrrolo[3,2-c]pyridine-7-carboxamide;
N-(4-(4-amino-7-cyano-3-(3-methoxy-4-((4-methylpyrimidin-2-yl)oxy)phenyl)-1H-pyrrolo[3,2-c]pyridin-2-yl)phenyl)acrylamide;
4-(2-(4-acrylamidophenyl)-4-amino-7-cyano-1H-pyrrolo[3,2-c]pyridin-3-yl)-N-cyclopropyl-2-methoxybenzamide;
4-(6-(4-acrylamidophenyl)-4-aminopyrazolo[5,1-f][1,2,4]triazin-5-yl)-2-(difluoromethoxy)-N-((1r, 3r)-3-fluorocyclobutyl)benzamide;
(S)-4-(6-(4-acrylamidophenyl)-4-aminopyrazolo[5,1-f][1,2,4]triazin-5-yl)-2-fluoro-N-(tetrahydrofuran-3-yl)benzamide;
4-(6-(4-acrylamidophenyl)-4-aminopyrazolo[5,1-f][1,2,4]triazin-5-yl)-N-cyclobutyl-2-methoxybenzamide;
4-(4-amino-6-(4-methacrylamidophenyl)pyrazolo[5,1-f][1,2,4]triazin-5-yl)-N-isobutylbenzamide;
N-(4-(4-amino-5-(3-methoxy-4-(pyrimidin-2-yloxy)phenyl)pyrazolo[5,1-f][1,2,4]triazin-6-yl)phenyl)acrylamide;
N-(4-(4-amino-5-(3-fluoro-4-(pyrimidin-2-yloxy)phenyl)pyrazolo[5,1-f][1,2,4]triazin-6-yl)phenyl)acrylamide;
N-(4-(4-amino-5-(4-((4-methylpyrimidin-2-yl)oxy)phenyl)pyrazolo[5,1-f][1,2,4]triazin-6-yl)phenyl)acrylamide;
N-(4-(4-amino-5-(4-phenoxyphenyl)pyrazolo[5,1-f][1,2,4]triazin-6-yl)phenyl) acrylamide;
N-(4-(4-amino-5-(4-(pyridin-2-yloxy)phenyl)pyrazolo[5,1-f][1,2,4]triazin-6-yl)phenyl)acrylamide;
N-(4-(4-amino-5-(4-(cyclopentyloxy)phenyl)pyrazolo[5,1-f][1,2,4]triazin-6-yl)phenyl)acrylamide;
N-(4-(4-amino-5-(4-(pyrimidin-2-yloxy)phenyl)pyrazolo[5,1-f][1,2,4]triazin-6-yl)-2-fluorophenyl)acrylamide;
N-(4-(4-amino-5-(4-(pyrimidin-2-yloxy)phenyl)pyrazolo[5,1-f][1,2,4]triazin-6-yl)-3-fluorophenyl)acrylamide;
4-(2-(4-acrylamidophenyl)-4-amino-7-cyano-1-(2-hydroxyethyl)-1H-pyrrolo[3,2-c]pyridin-3-yl)-N-cyclobutyl-2-methoxybenzamide;
4-(6-(4-acrylamidophenyl)-4-aminopyrazolo[5,1-f][1,2,4]triazin-5-yl)-N-isobutylbenzamide;
4-(6-(4-acrylamidophenyl)-4-aminopyrazolo[5,1-f][1,2,4]triazin-5-yl)-N-cyclobutylbenzamide;
4-(6-(4-acrylamidophenyl)-4-aminopyrazolo[5,1-f][1,2,4]triazin-5-yl)-N-cyclobutyl-2-fluorobenzamide;
methyl 4-(6-(4-acrylamidophenyl)-4-aminopyrazolo[5,1-f][1,2,4]triazin-5-yl)-2-methoxybenzoate;
4-(6-(4-acrylamidophenyl)-4-aminopyrazolo[5,1-f][1,2,4]triazin-5-yl)-2-methoxy-N-(2,2,2-trifluoroethyl)benzamide;
4-(6-(4-acrylamidophenyl)-4-aminopyrazolo[5,1-f][1,2,4]triazin-5-yl)-2-methoxy-N-(oxetan-3-yl)benzamide;
4-(6-(4-acrylamidophenyl)-4-aminopyrazolo[5,1-f][1,2,4]triazin-5-yl)-2-methoxy-N-((1-methyl-1H-pyrazol-3-yl)methyl)benzamide;
4-(6-(4-acrylamidophenyl)-4-aminopyrazolo[5,1-f][1,2,4]triazin-5-yl)-N-(cyclobutylmethyl)-2-methoxybenzamide;
4-(6-(4-acrylamidophenyl)-4-aminopyrazolo[5,1-f][1,2,4]triazin-5-yl)-N-((1s, 3s)-3-hydroxycyclobutyl)-2-methoxybenzamide;
4-(6-(4-acrylamidophenyl)-4-aminopyrazolo[5,1-f][1,2,4]triazin-5-yl)-N-((1-cyanocyclopropyl)methyl)-2-methoxybenzamide;
4-(4-amino-6-(4-(2-fluoroacrylamido)phenyl)pyrazolo[5,1-f][1,2,4]triazin-5-yl)-N-isobutylbenzamide;
4-(6-(4-acrylamidophenyl)-4-aminopyrazolo[5,1-f][1,2,4]triazin-5-yl)-N-cyclobutyl-2-methoxy-N-methylbenzamide;
4-(6-(4-acrylamidophenyl)-4-aminopyrazolo[5,1-f][1,2,4]triazin-5-yl)-N-cyclobutylcyclohex-3-ene-1-carboxamide;
4-(6-(4-acrylamido-3-fluorophenyl)-4-aminopyrazolo[5,1-f][1,2,4]triazin-5-yl)-N-isobutylbenzamide;
4-(6-(4-acrylamido-2-fluorophenyl)-4-aminopyrazolo[5,1-f][1,2,4]triazin-5-yl)-N-isobutylbenzamide;
4-(6-(4-acrylamido-3-methoxyphenyl)-4-aminopyrazolo[5,1-f][1,2,4]triazin-5-yl)-N-isobutylbenzamide;
4-(6-(4-acrylamido-2-methoxyphenyl)-4-aminopyrazolo[5,1-f][1,2,4]triazin-5-yl)-N-isobutylbenzamide;
4-(6-(4-acrylamidophenyl)-4-aminopyrazolo[5,1-f][1,2,4]triazin-5-yl)-N-cyclopentyl-2-methoxybenzamide;
4-(6-(4-acrylamidophenyl)-4-aminopyrazolo[5,1-f][1,2,4]triazin-5-yl)-2-methoxy-N-(2-methoxy-2-methylpropyl)benzamide;
4-(6-(4-acrylamidophenyl)-4-aminopyrazolo[5,1-f][1,2,4]triazin-5-yl)-N-(2-hydroxy-2-methylpropyl)-2-methoxybenzamide;
4-(6-(4-acrylamidophenyl)-4-aminopyrazolo[5,1-f][1,2,4]triazin-5-yl)-N-cyclohexyl-2-methoxybenzamide;
4-(2-(4-acrylamidophenyl)-4-amino-7-cyano-1-(2-methoxyethyl)-1H-pyrrolo[3,2-c]pyridin-3-yl)-N-cyclobutyl-2-methoxybenzamide;
4-(6-(4-acrylamidophenyl)-4-aminopyrazolo[5,1-f][1,2,4]triazin-5-yl)-N-ethyl-2-methoxybenzamide;
4-(6-(4-acrylamidophenyl)-4-aminopyrazolo[5,1-f][1,2,4]triazin-5-yl)-N-isopropyl-2-methoxybenzamide;
4-(6-(4-acrylamidophenyl)-4-aminopyrazolo[5,1-f][1,2,4]triazin-5-yl)-N-(2-(dimethylamino)ethyl)-2-methoxybenzamide;
4-(6-(4-acrylamidophenyl)-4-aminopyrazolo[5,1-f][1,2,4]triazin-5-yl)-2-methoxy-N-((1s, 3s)-3-methoxycyclobutyl)benzamide;
4-(2-(4-acrylamidophenyl)-4-amino-7-cyano-1-methyl-1H-pyrrolo[3,2-c]pyridin-3-yl)-N-isobutyl-2-methoxybenzamide;
4-(2-(4-acrylamidophenyl)-4-amino-7-cyano-1-methyl-1H-pyrrolo[3,2-c]pyridin-3-yl)-N-cyclobutylbenzamide;
4-(2-(4-acrylamidophenyl)-4-amino-7-cyano-1-methyl-1H-pyrrolo[3,2-c]pyridin-3-yl)-N-cyclobutyl-2-methoxybenzamide;
N-(4-(4-amino-7-cyano-1-methyl-3-(4-(pyrimidin-2-yloxy)phenyl)-1H-pyrrolo[3,2-c]pyridin-2-yl)phenyl)acrylamide;
N-(4-(4-amino-7-cyano-3-(3-methoxy-4-(pyrimidin-2-yloxy)phenyl)-1-methyl-1H-pyrrolo[3,2-c]pyridin-2-yl)phenyl)acrylamide;
N-(4-(4-amino-7-cyano-1-methyl-3-(4-((4-methylpyrimidin-2-yl)oxy)phenyl)-1H-pyrrolo[3,2-c]pyridin-2-yl)phenyl)acrylamide;
4-(4-amino-6-(4-methacrylamidophenyl)pyrazolo[5,1-f][1,2,4]triazin-5-yl)-2-methoxy-N-(2,2,2-trifluoroethyl)benzamide;
(E)-4-(4-amino-6-(4-(4-(dimethylamino)but-2-enamido)phenyl)pyrazolo[5,1-f][1,2,4]triazin-5-yl)-2-methoxy-N-(2,2,2-trifluoroethyl)benzamide;
4-(6-(4-acrylamidophenyl)-4-aminopyrazolo[5,1-f][1,2,4]triazin-5-yl)-N-cyclobutyl-2-(trifluoromethoxy)benzamide;
4-(6-(4-acrylamidophenyl)-4-aminopyrazolo[5,1-f][1,2,4]triazin-5-yl)-N-cyclobutyl-2-methylbenzamide;
4-(6-(4-acrylamidophenyl)-4-aminopyrazolo[5,1-f][1,2,4]triazin-5-yl)-N-cyclobutyl-2-ethoxybenzamide;
4-(6-(4-acrylamidophenyl)-4-aminopyrazolo[5,1-f][1,2,4]triazin-5-yl)-N-cyclobutyl-2-methoxy-5-methylbenzamide;
4-(4-amino-6-(4-(2-(trifluoromethyl)acrylamido)phenyl)pyrazolo[5,1-f][1,2,4]triazin-5-yl)-2-methoxy-N-(2,2,2-trifluoroethyl)benzamide;
4-(4-amino-6-(4-(N-methylacrylamido)phenyl)pyrazolo[5,1-f][1,2,4]triazin-5-yl)-2-methoxy-N-(2,2,2-trifluoroethyl)benzamide;
4-(6-(4-acrylamidophenyl)-4-aminopyrazolo[5,1-f][1,2,4]triazin-5-yl)-2-methoxy-N-methyl-N-(2,2,2-trifluoroethyl)benzamide;
N-(4-(4-amino-5-(3-methoxy-4-(4-(trifluoromethyl)oxazol-2-yl)phenyl)pyrazolo[5,1-f][1,2,4]triazin-6-yl)phenyl)acrylamide;
4-(6-(4-acrylamidophenyl)-4-aminopyrazolo[5,1-f][1,2,4]triazin-5-yl)-N-isobutyl-2-methoxybenzamide;
N-(4-(4-amino-5-(2,2-dimethyl-4-oxo-3,4-dihydro-2H-benzo[e][1,3]oxazin-7-yl)pyrazolo[5,1-f][1,2,4]triazin-6-yl)phenyl)acrylamide;
N-(4-(4-amino-5-(5-oxo-2,3,4,5-tetrahydrobenzo[f][1,4]oxazepin-8-yl)pyrazolo[5,1-f][1,2,4]triazin-6-yl)phenyl)acrylamide;
4-(6-(4-acrylamidophenyl)-4-aminopyrazolo[5,1-f][1,2,4]triazin-5-yl)-N-cyclobutyl-2-methoxy-6-methylbenzamide;
4-(6-(4-acrylamido-2-methoxyphenyl)-4-aminopyrazolo[5,1-f][1,2,4]triazin-5-yl)-2-methoxy-N-(2,2,2-trifluoroethyl)benzamide;
4-(6-(4-acrylamido-2-fluorophenyl)-4-aminopyrazolo[5,1-f][1,2,4]triazin-5-yl)-2-methoxy-N-(2,2,2-trifluoroethyl)benzamide;
4-(6-(4-acrylamido-2-methylphenyl)-4-aminopyrazolo[5,1-f][1,2,4]triazin-5-yl)-2-methoxy-N-(2,2,2-trifluoroethyl)benzamide;
4-(6-(2-acryloyl-2-azaspiro[3.3]heptan-6-yl)-4-aminopyrazolo[5,1-f][1,2,4]triazin-5-yl)-2-methoxy-N-(2,2,2-trifluoroethyl)benzamide;
4-(6-(4-acrylamidophenyl)-4-aminopyrazolo[5,1-f][1,2,4]triazin-5-yl)-N-cyclopropyl-2-methoxybenzamide;
4-(6-(6-acrylamidopyridin-3-yl)-4-aminopyrazolo[5,1-f][1,2,4]triazin-5-yl)-2-methoxy-N-(2,2,2-trifluoroethyl)benzamide;
4-(6-(4-acrylamidophenyl)-4-aminopyrazolo[5,1-f][1,2,4]triazin-5-yl)-2-(difluoromethoxy)-N-(2,2,2-trifluoroethyl)benzamide;
4-(4-amino-6-(4-(2-fluoroacrylamido)phenyl)pyrazolo[5,1-f][1,2,4]triazin-5-yl)-2-methoxy-N-methyl-N-(2,2,2-trifluoroethyl)benzamide;
4-(4-amino-6-(4-(2-fluoroacrylamido)phenyl)pyrazolo[5,1-f][1,2,4]triazin-5-yl)-N-cyclopentyl-2-methoxybenzamide;
4-(4-amino-6-(4-(2-fluoroacrylamido)phenyl)pyrazolo[5,1-f][1,2,4]triazin-5-yl)-N-ethyl-2-methoxybenzamide;
4-(4-amino-6-(4-(2-fluoroacrylamido)phenyl)pyrazolo[5,1-f][1,2,4]triazin-5-yl)-N-cyclobutyl-2-methoxybenzamide;
4-(4-amino-7-cyano-2-(4-(2-fluoroacrylamido)phenyl)-1-methyl-1H-pyrrolo[3,2-c]pyridin-3-yl)-N-cyclobutylbenzamide;
4-(6-(4-acrylamidophenyl)-4-aminopyrazolo[5,1-f][1,2,4]triazin-5-yl)-N-(3,3-difluorocyclobutyl)-2-methoxybenzamide;
4-(4-amino-6-(4-(2-fluoroacrylamido)phenyl)pyrazolo[5,1-f][1,2,4]triazin-5-yl)-N-(3,3-difluorocyclobutyl)-2-methoxybenzamide;
4-(4-amino-6-(4-(2-fluoroacrylamido)-2-methylphenyl)pyrazolo[5,1-f][1,2,4]triazin-5-yl)-2-methoxy-N-(2,2,2-trifluoroethyl)benzamide;
4-(4-amino-6-(4-(2-fluoroacrylamido)-2-methoxyphenyl)pyrazolo[5,1-f][1,2,4]triazin-5-yl)-2-methoxy-N-(2,2,2-trifluoroethyl)benzamide;
4-(4-amino-6-(4-(2-fluoroacrylamido)phenyl)pyrazolo[5,1-f][1,2,4]triazin-5-yl)-N-cyclopropyl-2-methoxybenzamide;
4-(4-amino-7-cyano-2-(4-(2-fluoroacrylamido)phenyl)-1-methyl-1H-pyrrolo[3,2-c]pyridin-3-yl)-N-cyclopropyl-2-methoxybenzamide;
4-(2-(4-acrylamidophenyl)-4-amino-7-cyano-1-methyl-1H-pyrrolo[3,2-c]pyridin-3-yl)-N-cyclopropyl-2-methoxybenzamide;
4-(2-(4-acrylamidophenyl)-4-amino-7-cyano-1-methyl-1H-pyrrolo[3,2-c]pyridin-3-yl)-N-(2,2,2-trifluoroethyl)benzamide;
4-(2-(4-acrylamidophenyl)-4-amino-7-cyano-1-methyl-1H-pyrrolo[3,2-c]pyridin-3-yl)-N-ethyl-2-methoxybenzamide;
4-(2-(4-acrylamidophenyl)-4-amino-7-cyano-1-methyl-1H-pyrrolo[3,2-c]pyridin-3-yl)-N-isopropyl-2-methoxybenzamide;
4-(2-(4-acrylamidophenyl)-4-amino-7-cyano-1-methyl-1H-pyrrolo[3,2-c]pyridin-3-yl)-N-cyclopentyl-2-methoxybenzamide;
4-(2-(4-acrylamidophenyl)-4-amino-7-cyano-1-methyl-1H-pyrrolo[3,2-c]pyridin-3-yl)-N-cyclohexyl-2-methoxybenzamide;
4-(2-(4-acrylamidophenyl)-4-amino-7-cyano-1-methyl-1H-pyrrolo[3,2-c]pyridin-3-yl)-N-((1-cyanocyclopropyl)methyl)-2-methoxybenzamide;
4-(2-(4-acrylamido-2-methoxyphenyl)-4-amino-7-cyano-1-methyl-1H-pyrrolo[3,2-c]pyridin-3-yl)-2-methoxy-N-(2,2,2-trifluoroethyl)benzamide;
5-(4-acrylamidophenyl)-4-(3-methoxy-4-((6-methylpyridin-2-yl)oxy)phenyl)nicotinamide;
5-(4-acrylamidophenyl)-2-amino-4-(3-methoxy-4-((6-methylpyridin-2-yl)oxy)phenyl)-6-methylnicotinamide;
N-(4-(4-amino-3-(3-methoxy-4-((6-methylpyridin-2-yl)oxy)phenyl)-7-oxo-6,7-dihydro-2H-pyrazolo[3,4-d]pyridazin-2-yl)phenyl)acrylamide;
N-(4-(4-amino-3-(3-methoxy-4-((6-methylpyridin-2-yl)oxy)phenyl)-7-oxo-6,7-dihydro-2H-pyrazolo[3,4-d]pyridazin-2-yl)phenyl)methacrylamide;
1-(3-((4-amino-5-(3-methoxy-4-((6-methylpyridin-2-yl)oxy)phenyl)-7H-pyrrolo[2,3-d]pyrimidin-7-yl)methyl)piperidin-1-yl)prop-2-en-1-one;
N-(4-(4-amino-3-(3-methoxy-4-((6-methylpyridin-2-yl)oxy)phenyl)-7-oxo-6,7-dihydro-2H-pyrazolo[3,4-d]pyridazin-2-yl)-2-fluorophenyl)acrylamide;
5-(4-acrylamidophenyl)-2-amino-4-(3-methoxy-4-((6-methylpyridin-2-yl)oxy)phenyl)-6-methylnicotinamide;
4-(4-amino-2-(4-methacrylamidophenyl)-7-oxo-6,7-dihydro-2H-pyrazolo[3,4-d]pyridazin-3-yl)-N-isobutylbenzamide;
4-(2-(4-acrylamidophenyl)-4-amino-7-oxo-6,7-dihydro-2H-pyrazolo[3,4-d]pyridazin-3-yl)-N-isobutylbenzamide;
N-(4-(4-cyano-6-(3-methoxy-4-((6-methylpyridin-2-yl)oxy)phenyl)-7-methyl-7H-pyrrolo[2,3-d]pyrimidin-5-yl)phenyl)acrylamide;
N-(4-(4-chloro-6-(3-methoxy-4-((6-methylpyridin-2-yl)oxy)phenyl)-7-methyl-7H-pyrrolo[2,3-d]pyrimidin-5-yl)phenyl)acrylamide;
N-(4-(4-methoxy-6-(3-methoxy-4-((6-methylpyridin-2-yl)oxy)phenyl)-7-methyl-7H-pyrrolo[2,3-d]pyrimidin-5-yl)phenyl)acrylamide;
4-(2-(4-acrylamidophenyl)-4-amino-7-oxo-6,7-dihydro-2H-pyrazolo[3,4-d]pyridazin-3-yl)-N-cyclobutylbenzamide;
N-(4-(4-amino-7-oxo-3-(4-(pyrrolidine-1-carbonyl)phenyl)-6,7-dihydro-2H-pyrazolo[3,4-d]pyridazin-2-yl)phenyl)acrylamide;
4-(2-(4-acrylamidophenyl)-4-amino-7-oxo-6,7-dihydro-2H-pyrazolo[3,4-d]pyridazin-3-yl)-N-(oxetan-3-yl)benzamide;
N-(4-(4-amino-3-(3-methoxy-4-((6-methylpyridin-2-yl)oxy)phenyl)-7-oxo-6,7-dihydro-2H-pyrazolo[3,4-d]pyridazin-2-yl)phenyl)-2-fluoroacrylamide;
N-(4-(4-amino-3-(4-(cyclopentyloxy)phenyl)-7-oxo-6,7-dihydro-2H-pyrazolo[3,4-d]pyridazin-2-yl)phenyl)acrylamide;
4-(2-(4-acrylamidophenyl)-4-amino-7-oxo-6,7-dihydro-2H-pyrazolo[3,4-d]pyridazin-3-yl)-N-(pyrrolidin-3-yl)benzamide;
4-(2-(4-acrylamidophenyl)-4-amino-7-oxo-6,7-dihydro-2H-pyrazolo[3,4-d]pyridazin-3-yl)-N-(tetrahydro-2H-pyran-3-yl)benzamide;
4-(2-(4-acrylamidophenyl)-4-amino-7-oxo-6,7-dihydro-2H-pyrazolo[3,4-d]pyridazin-3-yl)-N-cyclobutyl-2-methoxybenzamide;
N-(4-(4-amino-3-(3-methoxy-4-((4-methylpyrimidin-2-yl)oxy)phenyl)-7-oxo-6,7-dihydro-2H-pyrazolo[3,4-d]pyridazin-2-yl)phenyl)methacrylamide;
N-(4-(4-amino-3-(3-methoxy-4-((4-methylpyrimidin-2-yl)oxy)phenyl)-7-oxo-6,7-dihydro-2H-pyrazolo[3,4-d]pyridazin-2-yl)phenyl)acrylamide;
4-(2-(4-acrylamidophenyl)-4-amino-7-oxo-6,7-dihydro-2H-pyrazolo[3,4-d]pyridazin-3-yl)-N-isobutyl-2-methoxybenzamide;
N-(4-(4-amino-3-(4-((4-chloropyridin-2-yl)oxy)-3-methoxyphenyl)-7-oxo-6,7-dihydro-2H-pyrazolo[3,4-d]pyridazin-2-yl)phenyl)acrylamide;
4-(4-amino-2-(4-(2-fluoroacrylamido)phenyl)-7-oxo-6,7-dihydro-2H-pyrazolo[3,4-d]pyridazin-3-yl)-N-cyclobutyl-2-methoxybenzamide;
N-(4-(4-amino-3-(4-(cyclopentyloxy)-3-methoxyphenyl)-7-oxo-6,7-dihydro-2H-pyrazolo[3,4-d]pyridazin-2-yl)phenyl)acrylamide;
N-(4-(4-amino-3-(4-cyclobutoxyphenyl)-7-oxo-6,7-dihydro-2H-pyrazolo[3,4-d]pyridazin-2-yl)phenyl)acrylamide;
N-(4-(4-amino-3-(4-(cyclohexyloxy)phenyl)-7-oxo-6,7-dihydro-2H-pyrazolo[3,4-d]pyridazin-2-yl)phenyl)acrylamide;
N-(4-(4-amino-3-(4-isobutoxyphenyl)-7-oxo-6,7-dihydro-2H-pyrazolo[3,4-d]pyridazin-2-yl)phenyl)acrylamide;
N-(4-(4-amino-3-(4-(cyclopentyloxy)-3-fluorophenyl)-7-oxo-6,7-dihydro-2H-pyrazolo[3,4-d]pyridazin-2-yl)phenyl)acrylamide;
5-(4-acrylamidophenyl)-6-(3-methoxy-4-((6-methylpyridin-2-yl)oxy)phenyl)-1H-indazole-7-carboxamide;
N-(4-(4-amino-7-oxo-3-(4-((tetrahydro-2H-pyran-4-yl)oxy)phenyl)-6,7-dihydro-2H-pyrazolo[3,4-d]pyridazin-2-yl)phenyl)acrylamide;
N-(4-(4-amino-3-(6-(cyclohexyloxy)pyridin-3-yl)-7-oxo-6,7-dihydro-2H-pyrazolo[3,4-d]pyridazin-2-yl)phenyl)acrylamide;
N-(4-(4-amino-3-(4-(cyclohexyl(methyl)amino)phenyl)-7-oxo-6,7-dihydro-2H-pyrazolo[3,4-d]pyridazin-2-yl)phenyl)acrylamide;
N-(4-(4-amino-3-(4-((4,4-difluorocyclohexyl)oxy)phenyl)-7-oxo-6,7-dihydro-2H-pyrazolo[3,4-d]pyridazin-2-yl)phenyl)acrylamide;
N-(4-(4-amino-3-(4-(cyclohexylthio)phenyl)-7-oxo-6,7-dihydro-2H-pyrazolo[3,4-d]pyridazin-2-yl)phenyl)acrylamide;
N-(4-(4-amino-3-(4-(cyclopentyloxy)phenyl)-7-oxo-6,7-dihydro-2H-pyrazolo[3,4-d]pyridazin-2-yl)phenyl)methacrylamide;
4-(2-(4-acrylamido-2-fluorophenyl)-4-amino-7-cyano-1-methyl-1H-pyrrolo[3,2-c]pyridin-3-yl)-2-methoxy-N-(2,2,2-trifluoroethyl)benzamide;
4-(4-amino-7-cyano-2-(2-fluoro-4-(2-fluoroacrylamido)phenyl)-1-methyl-1H-pyrrolo[3,2-c]pyridin-3-yl)-2-methoxy-N-(2,2,2-trifluoroethyl)benzamide;
4-(4-amino-6-(4-(2-fluoroacrylamido)-2-methoxyphenyl)pyrazolo[5,1-f][1,2,4]triazin-5-yl)-N-cyclopropyl-2-methoxybenzamide;
4-(4-amino-6-(2-fluoro-4-(2-fluoroacrylamido)phenyl)pyrazolo[5,1-f][1,2,4]triazin-5-yl)-N-cyclopropyl-2-methoxybenzamide;
4-(6-(4-acrylamido-2-methoxyphenyl)-4-aminopyrazolo[5,1-f][1,2,4]triazin-5-yl)-N-cyclopropyl-2-methoxybenzamide;
4-(6-(4-acrylamido-2-fluorophenyl)-4-aminopyrazolo[5,1-f][1,2,4]triazin-5-yl)-N-cyclopropyl-2-methoxybenzamide;
4-(2-(4-acrylamido-2-methoxyphenyl)-4-amino-7-cyano-1-methyl-1H-pyrrolo[3,2-c]pyridin-3-yl)-N-cyclopropyl-2-methoxybenzamide;
4-(2-(4-acrylamido-2-fluorophenyl)-4-amino-7-cyano-1-methyl-1H-pyrrolo[3,2-c]pyridin-3-yl)-N-cyclopropyl-2-methoxybenzamide;
4-(4-amino-7-cyano-2-(4-(2-fluoroacrylamido)phenyl)-1-methyl-1H-pyrrolo[3,2-c]pyridin-3-yl)-N-isopropyl-2-methoxybenzamide; (S)-4-(4-amino-6-(4-(2-fluoroacrylamido)phenyl)pyrazolo[5,1-f][1,2,4]triazin-5-yl)-N-(3,3-difluorocyclopentyl)-2-methoxybenzamide;
4-(2-(4-acrylamidophenyl)-4-amino-7-cyano-1-methyl-1H-pyrrolo[3,2-c]pyridin-3-yl)-2-fluoro-N-(2,2,2-trifluoroethyl)benzamide;
N-(4-(4-amino-7-cyano-3-(4-(cyclopentyloxy)phenyl)-1-methyl-1H-pyrrolo[3,2-c]pyridin-2-yl)phenyl)acrylamide;
4-(6-(4-acrylamidophenyl)-4-aminopyrazolo[5,1-f][1,2,4]triazin-5-yl)-2-methoxy-N-(1-methylcyclopropyl)benzamide;
4-(4-amino-6-(4-(2-fluoroacrylamido)phenyl)pyrazolo[5,1-f][1,2,4]triazin-5-yl)-N-(cyclopropylmethyl)-2-methoxybenzamide;
N-(4-(4-amino-5-(4-(cyclopentyl(methyl)amino)phenyl)pyrazolo[5,1-f][1,2,4]triazin-6-yl)phenyl)acrylamide;
N-(4-(4-amino-5-(4-cyclobutoxyphenyl)pyrazolo[5,1-f][1,2,4]triazin-6-yl)phenyl)acrylamide;
4-(4-amino-6-(6-ethynylpyridin-3-yl)pyrazolo[5,1-f][1,2,4]triazin-5-yl)-2-methoxy-N-(2,2,2-trifluoroethyl)benzamide;
4-(2-(4-acrylamidophenyl)-4-amino-7-cyano-1-methyl-1H-pyrrolo[3,2-c]pyridin-3-yl)-N-(2,2-difluorocyclopropyl)-2-methoxybenzamide;
4-(2-(4-acrylamidophenyl)-4-amino-7-cyano-1-methyl-1H-pyrrolo[3,2-c]pyridin-3-yl)-N-(3-fluorocyclobutyl)-2-methoxybenzamide;
4-(2-(4-acrylamidophenyl)-4-amino-7-cyano-1-methyl-1H-pyrrolo[3,2-c]pyridin-3-yl)-2-methoxy-N-(1-methylcyclopropyl)benzamide;
4-(2-(4-acrylamidophenyl)-4-amino-7-cyano-1-methyl-1H-pyrrolo[3,2-c]pyridin-3-yl)-N-(cyclopropylmethyl)-2-methoxybenzamide;
4-(4-amino-7-cyano-2-(4-(2-fluoroacrylamido)-2-methoxyphenyl)-1-methyl-1H-pyrrolo[3,2-c]pyridin-3-yl)-N-cyclopropyl-2-methoxybenzamide;
N-(4-(4-amino-3-(4-cyclopropoxyphenyl)-7-oxo-6,7-dihydro-2H-pyrazolo[3,4-d]pyridazin-2-yl)phenyl)acrylamide;
N-(4-(4-amino-3-(4-((3-fluorocyclopentyl)oxy)phenyl)-7-oxo-6,7-dihydro-2H-pyrazolo[3,4-d]pyridazin-2-yl)phenyl)acrylamide;
N-(4-(4-amino-3-(4-((3,3-difluorocyclopentyl)oxy)phenyl)-7-oxo-6,7-dihydro-2H-pyrazolo[3,4-d]pyridazin-2-yl)phenyl)acrylamide;
N-(4-(4-amino-3-(4-((3-methylcyclohexyl)oxy)phenyl)-7-oxo-6,7-dihydro-2H-pyrazolo[3,4-d]pyridazin-2-yl)phenyl)acrylamide;
N-(4-(4-amino-3-(4-hydroxyphenyl)-7-oxo-6,7-dihydro-2H-pyrazolo[3,4-d]pyridazin-2-yl)phenyl)acrylamide;
N-(4-(4-amino-3-(4-(methoxymethoxy)phenyl)-7-oxo-6,7-dihydro-2H-pyrazolo[3,4-d]pyridazin-2-yl)phenyl)acrylamide;
N-(4-(4-amino-3-(1-(3,3-difluorocyclopentyl)-1H-indol-5-yl)-7-oxo-6,7-dihydro-2H-pyrazolo[3,4-d]pyridazin-2-yl)phenyl)acrylamide;
4-(4-amino-6-(6-ethynyl-4-methylpyridin-3-yl)pyrazolo[5,1-f][1,2,4]triazin-5-yl)-2-methoxy-N-(2,2,2-trifluoroethyl)benzamide;
4-(4-amino-7-cyano-2-(6-ethynylpyridin-3-yl)-1-methyl-1H-pyrrolo[3,2-c]pyridin-3-yl)-2-methoxy-N-(2,2,2-trifluoroethyl)benzamide;
4-(4-amino-7-cyano-2-(6-ethynylpyridin-3-yl)-1-methyl-1H-pyrrolo[3,2-c]pyridin-3-yl)-N-cyclopropyl-2-methoxybenzamide;
N-(4-(4-amino-5-(4-(cyclopentyloxy)-3-methoxyphenyl)pyrazolo[5,1-f][1,2,4]triazin-6-yl)phenyl)acrylamide;
4-(4-amino-6-(4-(2-fluoroacrylamido)phenyl)pyrazolo[5,1-f][1,2,4]triazin-5-yl)-2-methoxybenzamide;
4-(6-(4-acrylamidophenyl)-4-aminopyrazolo[5,1-f][1,2,4]triazin-5-yl)-N-(3-fluorocyclobutyl)-2-methoxybenzamide;
4-(4-amino-6-(4-(2-fluoroacrylamido)phenyl)pyrazolo[5,1-f][1,2,4]triazin-5-yl)-N-(3-fluorocyclobutyl)-2-methoxybenzamide;
4-(4-amino-6-(4-(2-fluoroacrylamido)phenyl)pyrazolo[5,1-f][1,2,4]triazin-5-yl)-2-methoxy-N-(1-methylcyclopropyl)benzamide;
4-(6-(4-acrylamido-2-chlorophenyl)-4-aminopyrazolo[5,1-f][1,2,4]triazin-5-yl)-N-cyclopropyl-2-methoxybenzamide;
4-(2-(4-acrylamidophenyl)-4-amino-7-cyano-1-methyl-1H-pyrrolo[3,2-c]pyridin-3-yl)-N-(3,3-difluorocyclobutyl)-2-methoxybenzamide;
4-(2-(4-acrylamidophenyl)-4-amino-7-cyano-1-methyl-1H-pyrrolo[3,2-c]pyridin-3-yl)-N-(2-fluoroethyl)-2-methoxybenzamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)phenyl)acrylamide;
N-(4-(4-amino-7-cyano-3-(3-methoxy-4-((6-methylpyridin-2-yl)oxy)phenyl)-1-methyl-1H-pyrrolo[3,2-c]pyridin-2-yl)phenyl)acrylamide;
4-(2-(1-(1-acryloylpiperidin-4-yl)-1H-pyrazol-4-yl)-4-amino-7-cyano-1-methyl-1H-pyrrolo[3,2-c]pyridin-3-yl)-2-methoxy-N-(2,2,2-trifluoroethyl)benzamide;
4-(4-amino-6-(4-(2-fluoroacrylamido)phenyl)pyrazolo[5,1-f][1,2,4]triazin-5-yl)-N-(2,2-difluorocyclopropyl)-2-methoxybenzamide;
4-(6-(4-acrylamidophenyl)-4-aminopyrazolo[5,1-f][1,2,4]triazin-5-yl)-N-(2-fluoroethyl)-2-methoxybenzamide;
4-(4-amino-6-(4-(2-fluoroacrylamido)phenyl)pyrazolo[5,1-f][1,2,4]triazin-5-yl)-N-(2-fluoroethyl)-2-methoxybenzamide;
4-(6-(4-acrylamidophenyl)-4-aminopyrazolo[5,1-f][1,2,4]triazin-5-yl)-N-(2,2-difluoroethyl)-2-methoxybenzamide;
4-(4-amino-6-(4-(2-fluoroacrylamido)phenyl)pyrazolo[5,1-f][1,2,4]triazin-5-yl)-N-(2,2-difluoroethyl)-2-methoxybenzamide;
4-(2-(4-acrylamidophenyl)-4-amino-7-cyano-1-methyl-1H-pyrrolo[3,2-c]pyridin-3-yl)-N-(2,2-difluoroethyl)-2-methoxybenzamide;
4-(4-amino-6-(2-chloro-4-(2-fluoroacrylamido)phenyl)pyrazolo[5,1-f][1,2,4]triazin-5-yl)-N-cyclopropyl-2-methoxybenzamide;
4-(2-(1-acryloylpyrrolidin-3-yl)-4-amino-7-cyano-1-methyl-1H-pyrrolo[3,2-c]pyridin-3-yl)-2-methoxy-N-(2,2,2-trifluoroethyl)benzamide;
4-(6-(1-(1-acryloylpiperidin-4-yl)-1H-pyrazol-4-yl)-4-aminopyrazolo[5,1-f][1,2,4]triazin-5-yl)-2-methoxy-N-(2,2,2-trifluoroethyl)benzamide;
N-(4-(4-amino-7-oxo-3-(4-(pyrrolidin-1-ylmethyl)phenyl)-6,7-dihydro-2H-pyrazolo[3,4-d]pyridazin-2-yl)phenyl)acrylamide;
4-amino-3-(4-(cyclohexyloxy)phenyl)-2-(6-ethynylpyridin-3-yl)-2,6-dihydro-7H-pyrazolo[3,4-d]pyridazin-7-one;
4-(4-amino-6-(4-(2-fluoroacrylamido)phenyl)pyrazolo[5,1-f][1,2,4]triazin-5-yl)-N-((1-fluorocyclopropyl)methyl)-2-methoxybenzamide;
N-(4-(4-amino-5-(3-fluoro-4-((4-methylpyrimidin-2-yl)oxy)phenyl)pyrazolo[5,1-f][1,2,4]triazin-6-yl)phenyl)acrylamide;
N-(4-(4-amino-5-(4-cyclobutoxyphenyl)pyrazolo[5,1-f][1,2,4]triazin-6-yl)phenyl)-2-fluoroacrylamide;
N-(4-(4-amino-5-(4-cyclobutoxyphenyl)pyrazolo[5,1-f][1,2,4]triazin-6-yl)phenyl)methacrylamide;
N-(4-(4-amino-5-(4-cyclobutoxy-3-fluorophenyl)pyrazolo[5,1-f][1,2,4]triazin-6-yl)phenyl)acrylamide;
4-(6-(1-acryloylpyrrolidin-3-yl)-4-aminopyrazolo[5,1-f][1,2,4]triazin-5-yl)-2-methoxy-N-(2,2,2-trifluoroethyl)benzamide;
4-(2-(4-acrylamidophenyl)-4-amino-7-cyano-1-methyl-1H-pyrrolo[3,2-c]pyridin-3-yl)-N-((1-fluorocyclopropyl)methyl)-2-methoxybenzamide;
4-(2-(4-acrylamido-3-fluorophenyl)-4-amino-7-cyano-1-methyl-1H-pyrrolo[3,2-c]pyridin-3-yl)-N-cyclopropyl-2-methoxybenzamide;
4-(2-(4-acrylamido-3-fluorophenyl)-4-amino-7-cyano-1-methyl-1H-pyrrolo[3,2-c]pyridin-3-yl)-2-methoxy-N-(2,2,2-trifluoroethyl)benzamide;
4-(2-(1-acryloyl-2,5-dihydro-1H-pyrrol-3-yl)-4-amino-7-cyano-1-methyl-1H-pyrrolo[3,2-c]pyridin-3-yl)-2-methoxy-N-(2,2,2-trifluoroethyl)benzamide;
N-(4-(4-amino-3-bromo-7-cyano-1-methyl-1H-pyrrolo[3,2-c]pyridin-2-yl)phenyl)-2-fluoroacrylamide;
1-(4-acrylamidophenyl)-3-amino-5-(3-methoxy-4-((6-methylpyridin-2-yl)oxy)phenyl)-1H-pyrazole-4-carboxamide;
N-(4-(4-amino-7-oxo-3-(4-(pyrrolidin-1-yl)phenyl)-6,7-dihydro-2H-pyrazolo[3,4-d]pyridazin-2-yl)phenyl)acrylamide;
N-(4-(4-amino-3-(3-methoxy-4-((6-methylpyridin-2-yl)oxy)phenyl)-2H-pyrazolo[3,4-d]pyrimidin-2-yl)phenyl)acrylamide;
N-(4-(4-amino-5-(6-cyclobutoxypyridin-3-yl)pyrazolo[5,1-f][1,2,4]triazin-6-yl)phenyl)acrylamide;
N-(4-(4-amino-5-(4-cyclobutoxyphenyl)pyrazolo[5,1-f][1,2,4]triazin-6-yl)-3-fluorophenyl)acrylamide;
N-(4-(4-amino-5-(4-cyclobutoxyphenyl)pyrazolo[5,1-f][1,2,4]triazin-6-yl)-3-methoxyphenyl)acrylamide;
N-(4-(4-amino-5-(4-(cyclopropylmethoxy)phenyl)pyrazolo[5,1-f][1,2,4]triazin-6-yl)phenyl)acrylamide;
N-(4-(4-amino-5-(4-cyclopropoxyphenyl)pyrazolo[5,1-f][1,2,4]triazin-6-yl)phenyl)acrylamide;
N-(4-(4-amino-5-(4-(cyclohexylthio)phenyl)pyrazolo[5,1-f][1,2,4]triazin-6-yl)phenyl)acrylamide;
N-(4-(4-amino-3-bromo-7-cyano-1-methyl-1H-pyrrolo[3,2-c]pyridin-2-yl)phenyl)acrylamide;
N-(4-(4-amino-5-(4-(2,2,2-trifluoroethoxy)phenyl)pyrazolo[5,1-f][1,2,4]triazin-6-yl)phenyl)acrylamide;
N-(4-(4-amino-5-(4-isopropoxyphenyl)pyrazolo[5,1-f][1,2,4]triazin-6-yl)phenyl)acrylamide;
N-(4-(4-amino-5-(4-(oxetan-3-yloxy)phenyl)pyrazolo[5,1-f][1,2,4]triazin-6-yl)phenyl)acrylamide;
N-(4-(4-amino-5-(4-(cyclohexyloxy)phenyl)pyrazolo[5,1-f][1,2,4]triazin-6-yl)phenyl)acrylamide;
N-(4-(4-amino-5-(4-((tetrahydro-2H-pyran-4-yl)oxy)phenyl)pyrazolo[5,1-f][1,2,4]triazin-6-yl)phenyl)acrylamide;
N-(4-(4-amino-5-(4-((3-methylcyclohexyl)oxy)phenyl)pyrazolo[5,1-f][1,2,4]triazin-6-yl)phenyl)acrylamide;
N-(4-(4-amino-5-(4-((4,4-dimethylcyclohexyl)oxy)phenyl)pyrazolo[5,1-f][1,2,4]triazin-6-yl)phenyl)acrylamide;
N-(4-(6-(4-acrylamidophenyl)-4-aminopyrazolo[5,1-f][1,2,4]triazin-5-yl)-2-methoxyphenyl)cyclopropanecarboxamide;
N-(4-(4-amino-5-(4-(1-(cyclopropylamino)-2,2,2-trifluoroethyl)-3-methoxyphenyl) pyrazolo[5,1-f][1,2,4]triazin-6-yl)phenyl)acrylamide;
4-(4-amino-6-(2-fluoro-4-(2-fluoroacrylamido)phenyl)pyrazolo[5,1-f][1,2,4]triazin-5-yl)-N-(3,3-difluorocyclobutyl)-2-methoxybenzamide;
4-(4-amino-7-cyano-2-(4-(2-fluoroacrylamido)phenyl)-1H-pyrrolo[3,2-c]pyridin-3-yl)-N-cyclopropyl-2-methoxybenzamide;
4-(2-(4-acrylamidophenyl)-4-amino-7-cyano-1-(2-fluoroethyl)-1H-pyrrolo[3,2-c]pyridin-3-yl)-N-cyclopropyl-2-methoxybenzamide;
N-(4-(2-(4-acrylamidophenyl)-4-amino-7-cyano-1-methyl-1H-pyrrolo[3,2-c]pyridin-3-yl)-2-methoxyphenyl)cyclopropanecarboxamide;
N-(4-(4-amino-7-cyano-3-(4-((5-fluoropyrimidin-2-yl)oxy)phenyl)-1-methyl-1H-pyrrolo[3,2-c]pyridin-2-yl)phenyl)acrylamide;
N-(4-(4-amino-5-(4-((3,3-difluorocyclopentyl)oxy)phenyl)pyrazolo[5,1-f][1,2,4]triazin-6-yl)phenyl)acrylamide;
N-(4-(4-amino-5-(4-cyclobutoxy-3-methoxyphenyl)pyrazolo[5,1-f][1,2,4]triazin-6-yl)phenyl)acrylamide;
N-(4-(4-amino-5-(4-(1-(cyclopropylamino)-2,2,2-trifluoroethyl)-3-methoxyphenyl) pyrazolo[5,1-f][1,2,4]triazin-6-yl)phenyl)-2-fluoroacrylamide;
4-(2-(4-acrylamidophenyl)-4-amino-7-cyano-1-ethyl-1H-pyrrolo[3,2-c]pyridin-3-yl)-N-cyclopropyl-2-methoxybenzamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)phenyl)acrylamide;
N-(4-(4-amino-7-cyano-3-(4-(1-(cyclopropylamino)-2,2,2-trifluoroethyl)-3-methoxyphenyl)-1-methyl-1H-pyrrolo[3,2-c]pyridin-2-yl)phenyl)acrylamide;
N-(4-(4-amino-5-(4-((4,4-difluorocyclohexyl)oxy)phenyl)pyrazolo[5,1-f][1,2,4]triazin-6-yl)phenyl)acrylamide;
N-(4-(4-amino-5-(4-((1-methylazetidin-3-yl)oxy)phenyl)pyrazolo[5,1-f][1,2,4]triazin-6-yl)phenyl)acrylamide;
N-(4-(4-amino-5-(4-(3-(dimethylamino)cyclobutoxy)phenyl)pyrazolo[5,1-f][1,2,4]triazin-6-yl)phenyl)acrylamide;
4-(2-(4-acrylamidophenyl)-4-amino-7-cyano-1H-pyrrolo[3,2-c]pyridin-3-yl)-2-methoxy-N-(2,2,2-trifluoroethyl)benzamide;
4-(4-amino-6-(2-fluoro-4-(2-fluoroacrylamido)phenyl)pyrazolo[5,1-f][1,2,4]triazin-5-yl)-N-(3-fluorocyclobutyl)-2-methoxybenzamide;
4-(4-amino-6-(4-(2-fluoroacrylamido)-2-methoxyphenyl)pyrazolo[5,1-f][1,2,4]triazin-5-yl)-N-((1-fluorocyclopropyl)methyl)-2-methoxybenzamide;
4-(4-amino-6-(4-(2-fluoroacrylamido)-2-methoxyphenyl)pyrazolo[5,1-f][1,2,4]triazin-5-yl)-N-(2,2-difluoroethyl)-2-methoxybenzamide;
4-(4-amino-6-(6-(2-fluoroacrylamido)pyridin-3-yl)pyrazolo[5,1-f][1,2,4]triazin-5-yl)-2-methoxy-N-(2,2,2-trifluoroethyl)benzamide;
4-(4-amino-6-(3-fluoro-4-(2-fluoroacrylamido)phenyl)pyrazolo[5,1-f][1,2,4]triazin-5-yl)-2-methoxy-N-(2,2,2-trifluoroethyl)benzamide;
4-(4-amino-6-(4-(2-fluoroacrylamido)-3-methoxyphenyl)pyrazolo[5,1-f][1,2,4]triazin-5-yl)-2-methoxy-N-(2,2,2-trifluoroethyl)benzamide;
4-(4-amino-6-(4-(2-fluoroacrylamido)-2-methoxyphenyl)pyrazolo[5,1-f][1,2,4]triazin-5-yl)-N-(3-fluorocyclobutyl)-2-methoxybenzamide;
4-(4-amino-6-(2-fluoro-4-(2-fluoroacrylamido)phenyl)pyrazolo[5,1-f][1,2,4]triazin-5-yl)-2-methoxy-N-(1-methylcyclopropyl)benzamide;
4-(4-amino-6-(4-(2-fluoroacrylamido)-2-methoxyphenyl)pyrazolo[5,1-f][1,2,4]triazin-5-yl)-2-methoxy-N-(1-methylcyclopropyl)benzamide;
4-(4-amino-6-(2-fluoro-4-(2-fluoroacrylamido)phenyl)pyrazolo[5,1-f][1,2,4]triazin-5-yl)-N-((1-fluorocyclopropyl)methyl)-2-methoxybenzamide;
4-(4-amino-6-(4-(2-fluoroacrylamido)-2-methoxyphenyl)pyrazolo[5,1-f][1,2,4]triazin-5-yl)-N-(2,2-difluorocyclopropyl)-2-methoxybenzamide;
N-(4-(4-amino-5-(4-(bicyclo[3.1.0]hexan-3-yloxy)phenyl)pyrazolo[5,1-f][1,2,4]triazin-6-yl)phenyl)acrylamide;
N-(4-(4-amino-5-(4-((3-fluorocyclopentyl)oxy)phenyl)pyrazolo[5,1-f][1,2,4]triazin-6-yl)phenyl)acrylamide
N-(5-(4-amino-5-(4-cyclobutoxyphenyl)pyrazolo[5,1-f][1,2,4]triazin-6-yl)pyridin-2-yl)acrylamide;
N-(4-(6-(4-acrylamidophenyl)-4-aminopyrazolo[5,1-f][1,2,4]triazin-5-yl)phenyl)cyclopropanecarboxamide;
N-(4-(4-amino-5-(4-(pyrrolidin-1-yl)phenyl)pyrazolo[5,1-f][1,2,4]triazin-6-yl)phenyl)acrylamide;
N-(4-(2-(4-acrylamidophenyl)-4-amino-7-cyano-1-methyl-1H-pyrrolo[3,2-c]pyridin-3-yl)-2-methoxyphenyl)cyclobutanecarboxamide;
N-(4-(2-(4-acrylamidophenyl)-4-amino-7-cyano-1-methyl-1H-pyrrolo[3,2-c]pyridin-3-yl)-2-methoxyphenyl)cyclopentanecarboxamide;
4-(4-amino-7-cyano-2-(2-fluoro-4-(2-fluoroacrylamido)phenyl)-1H-pyrrolo[3,2-c]pyridin-3-yl)-N-cyclopropyl-2-methoxybenzamide;
4-(6-(4-acrylamidophenyl)-4-aminopyrazolo[5,1-f][1,2,4]triazin-5-yl)-N-(1-cyanocyclopropyl)-2-methoxybenzamide;
4-(6-(4-acrylamidophenyl)-4-aminopyrazolo[5,1-f][1,2,4]triazin-5-yl)-N-(bicyclo[1.1.1]pentan-1-yl)-2-methoxybenzamide;
4-(4-amino-6-(4-(2-fluoroacrylamido)phenyl)pyrazolo[5,1-f][1,2,4]triazin-5-yl)-N-(bicyclo[1.1.1]pentan-1-yl)-2-methoxybenzamide;
4-(4-amino-6-(2-fluoro-4-(2-fluoroacrylamido)phenyl)pyrazolo[5,1-f][1,2,4]triazin-5-yl)-N-(2,2-difluorocyclopropyl)-2-methoxybenzamide;
N-(4-(6-(4-acrylamidophenyl)-4-aminopyrazolo[5,1-f][1,2,4]triazin-5-yl)-2-methoxyphenyl)cyclobutanecarboxamide;
N-(4-(4-amino-5-(4-(3-methoxycyclobutoxy)phenyl)pyrazolo[5,1-f][1,2,4]triazin-6-yl)phenyl)acrylamide;
N-(4-(4-amino-5-(4-(spiro[2.3]hexan-5-yloxy)phenyl)pyrazolo[5,1-f][1,2,4]triazin-6-yl)phenyl)acrylamide;
N-(4-(4-amino-5-(3-fluoro-4-((4-methylpyrimidin-2-yl)oxy)phenyl)pyrazolo[5,1-f][1,2,4]triazin-6-yl)phenyl)-2-fluoroacrylamide;
4-(2-(4-acrylamido-2-methoxyphenyl)-4-amino-7-cyano-1H-pyrrolo[3,2-c]pyridin-3-yl)-N-(3,3-difluorocyclobutyl)-2-methoxybenzamide;
N-(4-(4-amino-7-cyano-3-(3-methoxy-4-propionamidophenyl)-1-methyl-1H-pyrrolo[3,2-c]pyridin-2-yl)phenyl)acrylamide;
4-(2-(4-acrylamidophenyl)-4-amino-7-cyano-1-(2-fluoroethyl)-1H-pyrrolo[3,2-c]pyridin-3-yl)-N-(3,3-difluorocyclobutyl)-2-methoxybenzamide;
4-(4-amino-6-(4-(2-fluoroacrylamido)phenyl)pyrazolo[5,1-f][1,2,4]triazin-5-yl)-N-(1-cyanocyclopropyl)-2-methoxybenzamide;
N-(4-(4-amino-5-(4-(cyclobutylamino)phenyl)pyrazolo[5,1-f][1,2,4]triazin-6-yl)phenyl)acrylamide;
4-(2-(4-acrylamido-2-fluorophenyl)-4-amino-7-cyano-1H-pyrrolo[3,2-c]pyridin-3-yl)-N-(3,3-difluorocyclobutyl)-2-methoxybenzamide;
4-(2-(4-acrylamidophenyl)-4-amino-7-cyano-1-isopropyl-1H-pyrrolo[3,2-c]pyridin-3-yl)-N-(3,3-difluorocyclobutyl)-2-methoxybenzamide;
N-(4-(2-(4-acrylamidophenyl)-4-amino-7-cyano-1-methyl-1H-pyrrolo[3,2-c]pyridin-3-yl)-2-methoxyphenyl)-N-methylcyclopropanecarboxamide;
N-(4-(2-(4-acrylamidophenyl)-4-amino-7-cyano-1-methyl-1H-pyrrolo[3,2-c]pyridin-3-yl)phenyl)cyclopropanecarboxamide;
N-(4-(2-(4-acrylamidophenyl)-4-amino-7-cyano-1-methyl-1H-pyrrolo[3,2-c]pyridin-3-yl)-2-methoxyphenyl)-3,3-difluorocyclobutane-1-carboxamide;
N-(4-(2-(4-acrylamidophenyl)-4-amino-7-cyano-1-methyl-1H-pyrrolo[3,2-c]pyridin-3-yl)-2-methoxyphenyl)-1-methylcyclopropane-1-carboxamide;
N-(4-(2-(4-acrylamidophenyl)-4-amino-7-cyano-1-methyl-1H-pyrrolo[3,2-c]pyridin-3-yl)-2-methoxyphenyl)-1-fluorocyclopropane-1-carboxamide;
N-(4-(2-(4-acrylamidophenyl)-4-amino-7-cyano-1-methyl-1H-pyrrolo[3,2-c]pyridin-3-yl)-2-methoxyphenyl)-1-(trifluoromethyl)cyclopropane-1-carboxamide;
4-(4-amino-6-(2-fluoro-4-(2-fluoroacrylamido)phenyl)pyrazolo[5,1-f][1,2,4]triazin-5-yl)-N-(2,2-difluoroethyl)-2-methoxybenzamide;
N-(4-(4-amino-7-cyano-3-(4-cyclobutoxy-3-methoxyphenyl)-1-methyl-1H-pyrrolo[3,2-c]pyridin-2-yl)phenyl)acrylamide;
N-(4-(2-(4-acrylamidophenyl)-4-amino-7-cyano-1-methyl-1H-pyrrolo[3,2-c]pyridin-3-yl)-2-methoxyphenyl)-2,2-difluorocyclopropane-1-carboxamide;
N-(4-(4-amino-7-cyano-3-(4-(3-cyclopropylureido)-3-methoxyphenyl)-1-methyl-1H-pyrrolo[3,2-c]pyridin-2-yl)phenyl)acrylamide;
4-(4-amino-6-(4-(2-fluoroacrylamido)phenyl)pyrazolo[5,1-f][1,2,4]triazin-5-yl)-2-methoxy-N-(1,1,1-trifluoropropan-2-yl)benzamide;
4-(4-amino-6-(3-((dimethylamino)methyl)-4-(2-fluoroacrylamido)phenyl)pyrazolo[5,1-f][1,2,4]triazin-5-yl)-2-methoxy-N-(2,2,2-trifluoroethyl)benzamide;
N-(4-(4-amino-5-(4-(azetidin-1-yl)phenyl)pyrazolo[5,1-f][1,2,4]triazin-6-yl)phenyl)acrylamide;
N-(4-(4-amino-5-(4-(3-cyclopropylureido)-3-methoxyphenyl)pyrazolo[5,1-f][1,2,4]triazin-6-yl)phenyl)acrylamide;
N-(4-(6-(4-acrylamidophenyl)-4-aminopyrazolo[5,1-f][1,2,4]triazin-5-yl)-2-methoxyphenyl)azetidine-1-carboxamide;
N-(4-(4-amino-7-cyano-3-(4-(cyclobutylamino)phenyl)-1-methyl-1H-pyrrolo[3,2-c]pyridin-2-yl)phenyl)acrylamide;
N-(4-(2-(4-acrylamidophenyl)-4-amino-7-cyano-1-methyl-1H-pyrrolo[3,2-c]pyridin-3-yl)-2-methoxyphenyl)azetidine-1-carboxamide;
4-(2-(4-acrylamido-3-((dimethylamino)methyl)phenyl)-4-amino-7-cyano-1-methyl-1H-pyrrolo[3,2-c]pyridin-3-yl)-2-methoxy-N-(2,2,2-trifluoroethyl)benzamide;
N-(4-(4-amino-7-cyano-1-methyl-3-(4-((4-methylpyrimidin-2-yl)oxy)phenyl)-1H-pyrrolo[3,2-c]pyridin-2-yl)phenyl)-2-fluoroacrylamide;
4-(4-amino-7-cyano-2-(4-(2-fluoroacrylamido)phenyl)-1H-pyrrolo[3,2-c]pyridin-3-yl)-2-methoxy-N-(2,2,2-trifluoroethyl)benzamide;
4-(4-amino-7-cyano-2-(4-(2-fluoroacrylamido)phenyl)-1H-pyrrolo[3,2-c]pyridin-3-yl)-2-methoxy-N-(1-(trifluoromethyl)cyclopropyl)benzamide;
4-(2-(4-acrylamidophenyl)-4-amino-7-cyano-1-(2-methoxyethyl)-1H-pyrrolo[3,2-c]pyridin-3-yl)-N-(3,3-difluorocyclobutyl)-2-methoxybenzamide;
4-(2-(4-acrylamidophenyl)-4-amino-7-cyano-1-(2-fluoroethyl)-1H-pyrrolo[3,2-c]pyridin-3-yl)-2-methoxy-N-(2,2,2-trifluoroethyl)benzamide;
4-(2-(4-acrylamidophenyl)-4-amino-7-cyano-1-methyl-1H-pyrrolo[3,2-c]pyridin-3-yl)-2-methoxy-N-(1-(trifluoromethyl)cyclopropyl)benzamide;
4-(6-(4-acrylamidophenyl)-4-aminopyrazolo[5,1-f][1,2,4]triazin-5-yl)-2-methoxy-N-(1-(trifluoromethyl)cyclopropyl)benzamide;
4-(4-amino-6-(4-(2-fluoroacrylamido)phenyl)pyrazolo[5,1-f][1,2,4]triazin-5-yl)-2-methoxy-N-(1-(trifluoromethyl)cyclopropyl)benzamide;
4-(4-amino-6-(4-(2-fluoroacrylamido)phenyl)pyrazolo[5,1-f][1,2,4]triazin-5-yl)-2-methoxy-N-((1-(trifluoromethyl)cyclopropyl)methyl)benzamide;
4-(4-amino-6-(4-(2-fluoroacrylamido)phenyl)pyrazolo[5,1-f][1,2,4]triazin-5-yl)-2-methoxy-N-(3,3,3-trifluoropropyl)benzamide;
4-(6-(4-acrylamido-3-((dimethylamino)methyl)phenyl)-4-aminopyrazolo[5,1-f][1,2,4]triazin-5-yl)-2-methoxy-N-(2,2,2-trifluoroethyl)benzamide;
4-(6-(4-acrylamido-2-((dimethylamino)methyl)phenyl)-4-aminopyrazolo[5,1-f][1,2,4]triazin-5-yl)-2-methoxy-N-(2,2,2-trifluoroethyl)benzamide;
N-(4-(4-amino-5-(3-methoxy-4-(pyrrolidin-1-yl)phenyl)pyrazolo[5,1-f][1,2,4]triazin-6-yl)phenyl)acrylamide;
N-(4-(4-amino-5-(4-(cyclobutylamino)-3-methoxyphenyl)pyrazolo[5,1-f][1,2,4]triazin-6-yl)phenyl)acrylamide;
N-(4-(4-amino-7-cyano-1-methyl-3-(4-(pyrrolidin-1-yl)phenyl)-1H-pyrrolo[3,2-c]pyridin-2-yl)phenyl)acrylamide;
N-(4-(4-amino-7-cyano-3-(3-methoxy-4-(pyrrolidin-1-yl)phenyl)-1-methyl-1H-pyrrolo[3,2-c]pyridin-2-yl)phenyl)acrylamide;
N-(4-(4-amino-7-cyano-3-(4-(cyclobutylamino)-3-methoxyphenyl)-1-methyl-1H-pyrrolo[3,2-c]pyridin-2-yl)phenyl)acrylamide;
4-(2-(4-acrylamidophenyl)-4-amino-7-cyano-1-(2-methoxyethyl)-1H-pyrrolo[3,2-c]pyridin-3-yl)-2-methoxy-N-(2,2,2-trifluoroethyl)benzamide;
4-(4-amino-7-cyano-2-(4-(2-fluoroacrylamido)phenyl)-1H-pyrrolo[3,2-c]pyridin-3-yl)-N-(3,3-difluorocyclobutyl)-2-methoxybenzamide;
4-(4-amino-7-cyano-2-(4-(2-fluoroacrylamido)phenyl)-1H-pyrrolo[3,2-c]pyridin-3-yl)-N-(2,2-difluorocyclopropyl)-2-methoxybenzamide;
4-(4-amino-7-cyano-2-(4-(2-fluoroacrylamido)phenyl)-1H-pyrrolo[3,2-c]pyridin-3-yl)-2-methoxy-N-(1-methylcyclopropyl)benzamide;
N-(4-(4-amino-5-(1-methyl-1H-indol-5-yl)pyrazolo[5,1-f][1,2,4]triazin-6-yl)phenyl)acrylamide;
N-(4-(4-amino-5-(1-methyl-H-indol-5-yl)pyrazolo[5,1-f][1,2,4]triazin-6-yl)phenyl)-2-fluoroacrylamide;
4-(4-amino-6-(4-(2-fluoroacrylamido)phenyl)pyrazolo[5,1-f][1,2,4]triazin-5-yl)-N-(2,2,2-trifluoroethyl)benzamide;
N-(4-(4-amino-5-(4-((4-methylpyrimidin-2-yl)oxy)phenyl)pyrazolo[5,1-f][1,2,4]triazin-6-yl)phenyl)-2-fluoroacrylamide;
N-(4-(4-amino-7-cyano-1-methyl-3-(1-methyl-1H-indol-5-yl)-1H-pyrrolo[3,2-c]pyridin-2-yl)phenyl)acrylamide;
N-(4-(4-amino-3-(benzo[b]thiophen-2-yl)-7-cyano-1-methyl-1H-pyrrolo[3,2-c]pyridin-2-yl)phenyl)acrylamide;
4-(2-(4-acrylamidophenyl)-4-amino-7-cyano-1-isopropyl-1H-pyrrolo[3,2-c]pyridin-3-yl)-2-methoxy-N-(2,2,2-trifluoroethyl)benzamide;
4-(2-(4-acrylamidophenyl)-4-amino-7-cyano-1-(2,2,2-trifluoroethyl)-1H-pyrrolo[3,2-c]pyridin-3-yl)-2-methoxy-N-(2,2,2-trifluoroethyl)benzamide;
4-(4-amino-6-(4-(2-fluoroacrylamido)phenyl)pyrazolo[5,1-f][1,2,4]triazin-5-yl)-N-(3-fluorobicyclo[1.1.1]pentan-1-yl)-2-methoxybenzamide;
N-(4-(4-amino-5-(benzo[b]thiophen-2-yl)pyrazolo[5,1-f][1,2,4]triazin-6-yl)phenyl)-2-fluoroacrylamide;
N-(4-(4-amino-5-(benzo[b]thiophen-2-yl)pyrazolo[5,1-f][1,2,4]triazin-6-yl)phenyl)acrylamide;
4-(4-amino-6-(4-(2-fluoroacrylamido)phenyl)pyrazolo[5,1-f][1,2,4]triazin-5-yl)-N-((3,3-difluorocyclobutyl)methyl)-2-methoxybenzamide;
4-(4-amino-6-(4-(2-fluoroacrylamido)phenyl)pyrazolo[5,1-f][1,2,4]triazin-5-yl)-2-fluoro-N-(2,2,2-trifluoroethyl)benzamide;
N-(4-(4-amino-5-(3-fluoro-4-((5-fluoropyrimidin-2-yl)oxy)phenyl)pyrazolo[5,1-f][1,2,4]triazin-6-yl)phenyl)-2-fluoroacrylamide;
4-(4-amino-6-(4-(2-fluoroacrylamido)phenyl)pyrazolo[5,1-f][1,2,4]triazin-5-yl)-2-methoxy-N-(3-(trifluoromethyl)bicyclo[1.1.1]pentan-1-yl)benzamide;
4-(4-amino-6-(4-(2-fluoroacrylamido)phenyl)pyrazolo[5,1-f][1,2,4]triazin-5-yl)-N-(1,1-difluoropropan-2-yl)-2-methoxybenzamide;
N-(4-(6-(4-acrylamidophenyl)-4-aminopyrazolo[5,1-f][1,2,4]triazin-5-yl)-2-methoxyphenyl)-3,3-difluorocyclobutane-1-carboxamide;
4-(2-(4-acrylamidophenyl)-4-amino-7-cyano-1-methyl-1H-pyrrolo[3,2-c]pyridin-3-yl)-N-(bicyclo[1.1.1]pentan-1-yl)-2-methoxybenzamide;
4-(2-(4-acrylamidophenyl)-4-amino-7-cyano-1-(2,2,2-trifluoroethyl)-1H-pyrrolo[3,2-c]pyridin-3-yl)-N-cyclopropyl-2-methoxybenzamide;
N-(4-(2-(4-acrylamidophenyl)-4-amino-7-cyano-1-methyl-1H-pyrrolo[3,2-c]pyridin-3-yl)-2-fluorophenyl)cyclopropanecarboxamide;
4-(3-acrylamido-12-amino-9-cyano-6,7-dihydro-5H-benzo[c]pyrido[3′,4′:4,5]pyrrolo[1,2-a]azepin-13-yl)-N-(3,3-difluorocyclobutyl)-2-methoxybenzamide;
4-(4-amino-6-(4-(2-fluoroacrylamido)phenyl)pyrazolo[5,1-f][1,2,4]triazin-5-yl)-2-(methylthio)-N-(2,2,2-trifluoroethyl)benzamide;
N-(4-(4-amino-5-(2-(4-methoxyphenyl)thiazol-5-yl)pyrazolo[5,1-f][1,2,4]triazin-6-yl)phenyl)-2-fluoroacrylamide;
N-(4-(4-amino-5-(2-(4-methoxyphenyl)thiazol-5-yl)pyrazolo[5,1-f][1,2,4]triazin-6-yl)phenyl)acrylamide;
4-(2-(4-acrylamidophenyl)-4-amino-7-cyano-1-cyclopropyl-1H-pyrrolo[3,2-c]pyridin-3-yl)-2-methoxy-N-(2,2,2-trifluoroethyl)benzamide;
4-(2-(4-acrylamidophenyl)-4-amino-7-cyano-1-methyl-1H-pyrrolo[3,2-c]pyridin-3-yl)-2-(methylthio)-N-(2,2,2-trifluoroethyl)benzamide;
N-(4-(4-amino-5-(3-methoxy-4-(2-oxo-2-((2,2,2-trifluoroethyl)amino)ethyl)phenyl) pyrazolo[5,1-f][1,2,4]triazin-6-yl)phenyl)acrylamide;
N-(4-(4-amino-7-cyano-3-(3-methoxy-4-(2-oxo-2-((2,2,2-trifluoroethyl)amino)ethyl)phenyl)-1-methyl-1H-pyrrolo[3,2-c]pyridin-2-yl)phenyl)acrylamide;
4-(4-amino-6-(2-fluoro-4-(2-fluoroacrylamido)phenyl)pyrazolo[5,1-f][1,2,4]triazin-5-yl)-2-methoxy-N-(1-(trifluoromethyl)cyclopropyl)benzamide;
4-(4-amino-6-(2-fluoro-4-(2-fluoroacrylamido)phenyl)pyrazolo[5,1-f][1,2,4]triazin-5-yl)-N-(bicyclo[1.1.1]pentan-1-yl)-2-methoxybenzamide;
4-(4-amino-6-(2-fluoro-4-(2-fluoroacrylamido)phenyl)pyrazolo[5,1-f][1,2,4]triazin-5-yl)-2-methoxy-N-((1-(trifluoromethyl)cyclopropyl)methyl)benzamide;
4-(2-(4-acrylamidophenyl)-4-amino-7-cyano-1-cyclopropyl-1H-pyrrolo[3,2-c]pyridin-3-yl)-N-cyclopropyl-2-methoxybenzamide;
N-(4-(4-amino-5-(3-methoxy-4-(2-oxo-2-((2,2,2-trifluoroethyl)amino)ethyl)phenyl) pyrazolo[5,1-f][1,2,4]triazin-6-yl)phenyl)-2-fluoroacrylamide;
4-(4-amino-6-(2-fluoro-4-(2-fluoroacrylamido)phenyl)pyrazolo[5,1-f][1,2,4]triazin-5-yl)-2-methoxy-N-((1-(trifluoromethyl)cyclopropyl)methyl)benzamide;
4-(4-amino-6-(4-(2-fluoroacrylamido)phenyl)pyrazolo[5,1-f][1,2,4]triazin-5-yl)-N-(2,2-difluoropropyl)-2-methoxybenzamide;
N-(4-(4-amino-5-(4-(azetidine-1-carbonyl)-3-methoxyphenyl)pyrazolo[5,1-f][1,2,4]triazin-6-yl)phenyl)-2-fluoroacrylamide;
N-(4-(4-amino-5-(3-fluoro-4-((6-methylpyridin-2-yl)oxy)phenyl)pyrazolo[5,1-f][1,2,4]triazin-6-yl)phenyl)-2-fluoroacrylamide;
N-(4-(4-amino-5-(3-fluoro-4-((6-methylpyridin-2-yl)oxy)phenyl)pyrazolo[5,1-f][1,2,4]triazin-6-yl)phenyl)acrylamide;
N-(4-(4-amino-5-(3-fluoro-4-((2-methylpyrimidin-4-yl)oxy)phenyl)pyrazolo[5,1-f][1,2,4]triazin-6-yl)phenyl)-2-fluoroacrylamide;
N-(4-(4-amino-5-(3-fluoro-4-((2-methylpyrimidin-4-yl)oxy)phenyl)pyrazolo[5,1-f][1,2,4]triazin-6-yl)phenyl)acrylamide;
N-(4-(4-amino-5-(1-oxo-2-(2,2,2-trifluoroethyl)-1,2-dihydroisoquinolin-6-yl)pyrazolo[5,1-f][1,2,4]triazin-6-yl)phenyl)acrylamide;
N-(4-(4-amino-5-(1-oxo-2-(2,2,2-trifluoroethyl)isoindolin-5-yl)pyrazolo[5,1-f][1,2,4]triazin-6-yl)phenyl)acrylamide;
4-(4-amino-6-(4-(2-fluoroacrylamido)phenyl)pyrazolo[5,1-f][1,2,4]triazin-5-yl)-N-((1-fluorocyclopropyl)methyl)-2-(methoxy-d3)benzamide;
N-(4-(4-amino-5-(3-fluoro-4-((6-methylpyrazin-2-yl)oxy)phenyl)pyrazolo[5,1-f][1,2,4]triazin-6-yl)phenyl)-2-fluoroacrylamide;
N-(4-(4-amino-5-(3-fluoro-4-((6-methylpyrazin-2-yl)oxy)phenyl)pyrazolo[5,1-f][1,2,4]triazin-6-yl)phenyl)acrylamide;
N-(4-(4-amino-5-(3-fluoro-4-((5-methylpyrimidin-2-yl)oxy)phenyl)pyrazolo[5,1-f][1,2,4]triazin-6-yl)phenyl)-2-fluoroacrylamide;
N-(4-(4-amino-5-(3-fluoro-4-((5-methylpyrimidin-2-yl)oxy)phenyl)pyrazolo[5,1-f][1,2,4]triazin-6-yl)phenyl)acrylamide;
N-(4-(4-amino-5-(3-fluoro-4-(pyrimidin-2-yloxy)phenyl)pyrazolo[5,1-f][1,2,4]triazin-6-yl)phenyl)-2-fluoroacrylamide;
N-(4-(4-amino-3-(3-fluoro-4-((4-methylpyrimidin-2-yl)oxy)phenyl)-2H-pyrazolo[3,4-d]pyrimidin-2-yl)phenyl)methacrylamide;
N-(4-(4-amino-5-(3-fluoro-4-((4-methoxypyrimidin-2-yl)oxy)phenyl)pyrazolo[5,1-f][1,2,4]triazin-6-yl)phenyl)-2-fluoroacrylamide;
N-(4-(4-amino-5-(4-((4-(difluoromethyl)pyrimidin-2-yl)oxy)-3-fluorophenyl)pyrazolo[5,1-f][1,2,4]triazin-6-yl)phenyl)-2-fluoroacrylamide;
N-(4-(4-amino-5-(3-fluoro-4-((6-methylpyrimidin-4-yl)oxy)phenyl)pyrazolo[5,1-f][1,2,4]triazin-6-yl)phenyl)-2-fluoroacrylamide;
N-(4-(4-amino-5-(3-fluoro-4-((6-methylpyrimidin-4-yl)oxy)phenyl)pyrazolo[5,1-f][1,2,4]triazin-6-yl)phenyl)acrylamide;
4-(4-amino-6-(4-(2-fluoroacrylamido)phenyl)pyrazolo[5,1-f][1,2,4]triazin-5-yl)-N-((1-fluorocyclopropyl)methyl)benzamide;
N-(4-(4-amino-5-(3-fluoro-4-((4-methylpyrimidin-2-yl)thio)phenyl)pyrazolo[5,1-f][1,2,4]triazin-6-yl)phenyl)-2-fluoroacrylamide;
N-(4-(4-amino-5-(3-fluoro-4-((4-methylpyrimidin-2-yl)thio)phenyl)pyrazolo[5,1-f][1,2,4]triazin-6-yl)phenyl)-2-fluoroacrylamide;
N-(4-(4-amino-5-(4-((4-cyclopropylpyrimidin-2-yl)oxy)-3-fluorophenyl)pyrazolo[5,1-f][1,2,4]triazin-6-yl)phenyl)-2-fluoroacrylamide
N-(4-(4-amino-5-(4-((4-cyclopropylpyrimidin-2-yl)oxy)-3-fluorophenyl)pyrazolo[5,1-f][1,2,4]triazin-6-yl)phenyl)acrylamide;
N-(4-(4-amino-5-(3-fluoro-4-(imidazo[1,2-c]pyrimidin-5-yloxy)phenyl)pyrazolo[5,1-f][1,2,4]triazin-6-yl)phenyl)-2-fluoroacrylamide;
N-(4-(4-amino-5-(3-fluoro-4-((4-methylpyrimidin-2-yl)oxy)phenyl)pyrazolo[5,1-f][1,2,4]triazin-6-yl)-3-fluorophenyl)methacrylamide;
4-(4-amino-6-(4-(2-fluoroacrylamido)phenyl)pyrazolo[5,1-f][1,2,4]triazin-5-yl)-2-methoxy-N-phenylbenzamide;
4-(4-amino-6-(4-(2-fluoroacrylamido)phenyl)pyrazolo[5,1-f][1,2,4]triazin-5-yl)-2-methoxy-N-(pyridin-2-yl)benzamide;
N-(4-(4-amino-5-(3-fluoro-4-(methyl(4-methylpyrimidin-2-yl)amino)phenyl) pyrazolo[5,1-f][1,2,4]triazin-6-yl)phenyl)-2-fluoroacrylamide;
N-(4-(4-amino-5-(3-fluoro-4-((4-(trifluoromethyl)pyrimidin-2-yl)oxy)phenyl) pyrazolo[5,1-f][1,2,4]triazin-6-yl)phenyl)-2-fluoroacrylamide;
N-(4-(4-amino-5-(3-fluoro-4-((4-(trifluoromethyl)pyrimidin-2-yl)oxy)phenyl)pyrazolo[5,1-f][1,2,4]triazin-6-yl)phenyl)acrylamide;
4-(4-amino-6-(4-(but-2-ynamido)phenyl)pyrazolo[5,1-f][1,2,4]triazin-5-yl)-2-methoxy-N-(1-(trifluoromethyl)cyclopropyl)benzamide;
4-(4-amino-6-(4-propiolamidophenyl)pyrazolo[5,1-f][1,2,4]triazin-5-yl)-2-methoxy-N-(1-(trifluoromethyl)cyclopropyl)benzamide;
4-(4-amino-6-(4-(2-fluoroacrylamido)phenyl)pyrazolo[5,1-f][1,2,4]triazin-5-yl)-N-(tert-butyl)-2-methoxybenzamide;
N-(4-(4-amino-5-(4-((4-ethylpyrimidin-2-yl)oxy)-3-fluorophenyl)pyrazolo[5,1-f][1,2,4]triazin-6-yl)phenyl)-2-fluoroacrylamide;
N-(4-(4-amino-5-(4-((4-ethylpyrimidin-2-yl)oxy)-3-fluorophenyl)pyrazolo[5,1-f][1,2,4]triazin-6-yl)phenyl)acrylamide;
N-(4-(4-amino-5-(3-fluoro-4-((1-methyl-1H-pyrazol-3-yl)oxy)phenyl)pyrazolo[5,1-f][1,2,4]triazin-6-yl)phenyl)-2-fluoroacrylamide;
N-(4-(4-amino-5-(3-fluoro-4-((1-methyl-1H-pyrazol-3-yl)oxy)phenyl)pyrazolo[5,1-f][1,2,4]triazin-6-yl)phenyl)acrylamide;
N-(4-(4-amino-5-(3-fluoro-4-((4-methylpyrimidin-2-yl)amino)phenyl)pyrazolo[5,1-f][1,2,4]triazin-6-yl)phenyl)-2-fluoroacrylamide;
N-(4-(4-amino-5-(3-fluoro-4-((4-methylpyrimidin-2-yl)amino)phenyl)pyrazolo[5,1-f][1,2,4]triazin-6-yl)phenyl)acrylamide;
N-(4-(4-amino-5-(3-fluoro-4-((4-methylpyridin-2-yl)oxy)phenyl)pyrazolo[5,1-f][1,2,4]triazin-6-yl)phenyl)-2-fluoroacrylamide;
N-(4-(4-amino-5-(3-fluoro-4-((4-methylpyridin-2-yl)oxy)phenyl)pyrazolo[5,1-f][1,2,4]triazin-6-yl)phenyl)acrylamide;
N-(4-(4-amino-5-(4-(4-fluorobenzoyl)-3-methoxyphenyl)pyrazolo[5,1-f][1,2,4]triazin-6-yl)phenyl)acrylamide;
N-(4-(4-amino-5-(3-fluoro-4-((6-methylpyridin-2-yl)oxy)phenyl)pyrazolo[5,1-f][1,2,4]triazin-6-yl)phenyl)methacrylamide;
N-(4-(4-amino-5-(3-fluoro-4-((6-methylpyridin-2-yl)oxy)phenyl)pyrazolo[5,1-f][1,2,4]triazin-6-yl)phenyl)but-2-ynamide;
N-(4-(4-amino-5-(3-fluoro-4-((6-methylpyridin-2-yl)oxy)phenyl)pyrazolo[5,1-f][1,2,4]triazin-6-yl)phenyl)propiolamide;
N-(4-(4-amino-5-(3-fluoro-4-((6-methylpyridin-2-yl)oxy)phenyl)pyrazolo[5,1-f][1,2,4]triazin-6-yl)phenyl)ethenesulfonamide;
(E)-N-(4-(4-amino-5-(3-fluoro-4-((6-methylpyridin-2-yl)oxy)phenyl)pyrazolo[5,1-f][1,2,4]triazin-6-yl)phenyl)-4-(dimethylamino)but-2-enamide;
1-(4-(4-amino-5-(3-fluoro-4-((6-methylpyridin-2-yl)oxy)phenyl)pyrazolo[5,1-f][1,2,4]triazin-6-yl)-3,6-dihydropyridin-1(2H)-yl)prop-2-en-1-one;
(E)-N-(4-(4-amino-5-(3-fluoro-4-((6-methylpyridin-2-yl)oxy)phenyl)pyrazolo[5,1-f][1,2,4]triazin-6-yl)phenyl)but-2-enamide
4-(4-amino-6-(4-(2-fluoroacrylamido)-2-methoxyphenyl)pyrazolo[5,1-f][1,2,4]triazin-5-yl)-2-methoxy-N-(1-(trifluoromethyl)cyclopropyl)benzamide;
4-(4-amino-6-(4-(2-fluoroacrylamido)-2-methoxyphenyl)pyrazolo[5,1-f][1,2,4]triazin-5-yl)-N-(3-fluorobicyclo[1.1.1]pentan-1-yl)-2-methoxybenzamide;
N-(4-(4-amino-5-(3-fluoro-4-(methyl(4-methylpyrimidin-2-yl)amino)phenyl)pyrazolo[5,1-f][1,2,4]triazin-6-yl)phenyl)acrylamide;
N-(4-(4-amino-5-(4-benzoyl-3-methoxyphenyl)pyrazolo[5,1-f][1,2,4]triazin-6-yl)phenyl) acrylamide;
N-(4-(4-amino-5-(4-(4,4-difluorocyclohexane-1-carbonyl)-3-methoxyphenyl)pyrazolo[5,1-f][1,2,4]triazin-6-yl)phenyl)acrylamide;
1-(4-(4-amino-5-(3-fluoro-4-((6-methylpyridin-2-yl)oxy)phenyl)pyrazolo[5,1-f][1,2,4]triazin-6-yl)piperidin-1-yl)prop-2-en-1-one;
1-(4-(4-(4-amino-5-(3-fluoro-4-((6-methylpyridin-2-yl)oxy)phenyl)pyrazolo[5,1-f][1,2,4]triazin-6-yl)-1H-pyrazol-1-yl)piperidin-1-yl)prop-2-en-1-one;
N-(4-(4-amino-5-(3-fluoro-4-((6-methylpyridin-2-yl)oxy)phenyl)pyrazolo[5,1-f][1,2,4]triazin-6-yl)phenyl)-2-((dimethylamino)methyl)acrylamide;
N-(4-(4-amino-5-(3-fluoro-4-((6-methylpyridin-2-yl)oxy)phenyl)pyrazolo[5,1-f][1,2,4]triazin-6-yl)phenyl)-2-chloroacetamide;
N-(4-(4-amino-5-(3-fluoro-4-((6-methylpyridin-2-yl)oxy)phenyl)pyrazolo[5,1-f][1,2,4]triazin-6-yl)phenyl)-2-chloroacrylamide;
(Z)-4-((4-(4-amino-5-(3-fluoro-4-((6-methylpyridin-2-yl)oxy)phenyl)pyrazolo[5,1-f][1,2,4]triazin-6-yl)phenyl)amino)-4-oxobut-2-enoic acid;
4-(4-amino-6-(4-(2-fluoroacrylamido)-2-methoxyphenyl)pyrazolo[5,1-f][1,2,4]triazin-5-yl)-N-(bicyclo[1.1.1]pentan-1-yl)-2-methoxybenzamide;
4-(4-amino-6-(2-fluoro-4-(2-fluoroacrylamido)phenyl)pyrazolo[5,1-f][1,2,4]triazin-5-yl)-N-(3-fluorobicyclo[1.1.1]pentan-1-yl)-2-methoxybenzamide;
N-(4-(4-amino-5-(4-((4-aminopyrimidin-2-yl)oxy)-3-fluorophenyl)pyrazolo[5,1-f][1,2,4]triazin-6-yl)phenyl)acrylamide;
4-(4-amino-6-(2-fluoro-4-methacrylamidophenyl)pyrazolo[5,1-f][1,2,4]triazin-5-yl)-2-methoxy-N-(1-(trifluoromethyl)cyclopropyl)benzamide;
N-(4-(4-amino-7-cyano-3-(3-fluoro-4-((6-methylpyridin-2-yl)oxy)phenyl)-1-methyl-1H-pyrrolo[3,2-c]pyridin-2-yl)phenyl)acrylamide;
N-(4-(4-amino-5-(4-(cyclohexanecarbonyl)-3-methoxyphenyl)pyrazolo[5,1-f][1,2,4]triazin-6-yl)phenyl)acrylamide;
4-(4-amino-6-(4-(2-fluoroacrylamido)-2-(trifluoromethyl)phenyl)pyrazolo[5,1-f][1,2,4]triazin-5-yl)-2-methoxy-N-(1-(trifluoromethyl)cyclopropyl)benzamide;
N-(4-(4-amino-5-(3-fluoro-4-((4-(methylamino)pyrimidin-2-yl)oxy)phenyl)pyrazolo[5,1-f][1,2,4]triazin-6-yl)phenyl)acrylamide;
N-(4-(4-amino-5-(3-fluoro-4-((6-methylpyridin-2-yl)oxy)phenyl)pyrazolo[5,1-f][1,2,4]triazin-6-yl)-3-fluorophenyl)methacrylamide;
N-(4-(4-amino-7-cyano-3-(4-((4-(difluoromethyl)pyrimidin-2-yl)oxy)-3-fluorophenyl)-1-methyl-1H-pyrrolo[3,2-c]pyridin-2-yl)phenyl)acrylamide;
N-(4-(4-amino-7-cyano-3-(3-fluoro-4-((5-fluoro-4-methylpyrimidin-2-yl)oxy)phenyl)-1-methyl-1H-pyrrolo[3,2-c]pyridin-2-yl)phenyl)acrylamide;
N-(4-(4-amino-7-cyano-1-methyl-3-(4-((6-methylpyridin-2-yl)oxy)phenyl)-1H-pyrrolo[3,2-c]pyridin-2-yl)phenyl)acrylamide;
N-(4-(4-amino-5-(3-fluoro-4-((6-methylpyridin-2-yl)oxy)phenyl)pyrazolo[5,1-f][1,2,4]triazin-6-yl)-3-fluorophenyl)-2-fluoroacrylamide;
N-(4-(4-amino-5-(3-fluoro-4-((6-methylpyridin-2-yl)oxy)phenyl)pyrazolo[5,1-f][1,2,4]triazin-6-yl)-3-methylphenyl)-2-fluoroacrylamide;
N-(4-(4-amino-7-cyano-3-(3-fluoro-4-((1-methyl-1H-pyrazol-3-yl)oxy)phenyl)-1-methyl-1H-pyrrolo[3,2-c]pyridin-2-yl)phenyl)acrylamide;
N-(4-(4-amino-5-(2-(1-methylcyclopropyl)benzo[d]thiazol-6-yl)pyrazolo[5,1-f][1,2,4]triazin-6-yl)phenyl)acrylamide;
N-(4-(4-amino-5-(3-methoxy-4-((1-methylcyclohexyl)oxy)phenyl)pyrazolo[5,1-f][1,2,4]triazin-6-yl)phenyl)acrylamide;
N-(4-(4-amino-7-cyano-3-(3-fluoro-4-(pyrimidin-2-yloxy)phenyl)-1-methyl-1H-pyrrolo[3,2-c]pyridin-2-yl)phenyl)acrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)phenyl)acrylamide;
4-(4-amino-6-(2-chloro-4-(2-fluoroacrylamido)phenyl)pyrazolo[5,1-f][1,2,4]triazin-5-yl)-2-methoxy-N-(1-(trifluoromethyl)cyclopropyl)benzamide;
4-(4-amino-6-(4-(2-fluoroacrylamido)-2-methylphenyl)pyrazolo[5,1-f][1,2,4]triazin-5-yl)-2-methoxy-N-(1-(trifluoromethyl)cyclopropyl)benzamide;
4-(4-amino-6-(2-chloro-4-(2-fluoroacrylamido)phenyl)pyrazolo[5,1-f][1,2,4]triazin-5-yl)-2-methoxy-N-(1-(trifl4-(4-amino-6-(2-chloro-4-(2-fluoroacrylamido)phenyl)pyrazolo[5,1-f][1,2,4]triazin-5-yl)-2-methoxy-N-(1-(trifluoromethyl)cyclopropyl)benzamide;
N-(4-(4-amino-5-(3-fluoro-4-((6-methylpyridin-2-yl)oxy)phenyl)pyrazolo[5,1-f][1,2,4]triazin-6-yl)-3-chlorophenyl)-2-fluoroacrylamide;
N-(4-(4-amino-5-(3-fluoro-4-((4-methylpyrimidin-2-yl)oxy)phenyl)pyrazolo[5,1-f][1,2,4]triazin-6-yl)-3-methoxyphenyl)methacrylamide;
N-(4-(4-amino-5-(3-fluoro-4-((4-methylpyrimidin-2-yl)oxy)phenyl)pyrazolo[5,1-f][1,2,4]triazin-6-yl)-3-methylphenyl)methacrylamide;
N-(4-(4-amino-5-(3-fluoro-4-((4-methylpyrimidin-2-yl)oxy)phenyl)pyrazolo[5,1-f][1,2,4]triazin-6-yl)-3-chlorophenyl)methacrylamide;
N-(4-(4-amino-5-(3-fluoro-4-((1-methyl-1H-pyrazol-3-yl)oxy)phenyl)pyrazolo[5,1-f][1,2,4]triazin-6-yl)phenyl)methacrylamide;
N-(4-(4-amino-5-(3-fluoro-4-((1-methyl-1H-pyrazol-3-yl)oxy)phenyl)pyrazolo[5,1-f][1,2,4]triazin-6-yl)-3-fluorophenyl)methacrylamide;
N-(4-(4-amino-5-(3-fluoro-4-((1-methyl-1H-pyrazol-3-yl)oxy)phenyl)pyrazolo[5,1-f][1,2,4]triazin-6-yl)phenyl)but-2-ynamide;
N-(4-(4-amino-5-(3-fluoro-4-((1-methyl-1H-pyrazol-3-yl)oxy)phenyl)pyrazolo[5,1-f][1,2,4]triazin-6-yl)phenyl)propiolamide;
N-(4-(4-amino-5-(3-methoxy-4-(1-methyl-1H-imidazole-2-carbonyl)phenyl) pyrazolo[5,1-f][1,2,4]triazin-6-yl)phenyl)acrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)phenyl)acrylamide;
N-(4-(4-amino-7-cyano-3-(4-((5-fluoropyrimidin-2-yl)oxy)phenyl)-1-methyl-1H-pyrrolo[3,2-c]pyridin-2-yl)-3-fluorophenyl)methacrylamide;
N-(4-(4-amino-5-(3-fluoro-4-((6-methylpyridin-2-yl)oxy)phenyl)pyrazolo[5,1-f][1,2,4]triazin-6-yl)-3-methoxyphenyl)-2-fluoroacrylamide;
N-(4-(4-amino-7-cyano-3-(4-((4-(difluoromethyl)pyrimidin-2-yl)oxy)-3-fluorophenyl)-1-methyl-1H-pyrrolo[3,2-c]pyridin-2-yl)-3-fluorophenyl)methacrylamide;
N-(4-(4-amino-7-cyano-3-(3-fluoro-4-((5-fluoro-4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-(pyrimidin-2-yloxy)phenyl)-1-methyl-1H-pyrrolo[3,2-c]pyridin-2-yl)-3-fluorophenyl)methacrylamide;
N-(4-(4-amino-7-cyano-3-(4-((4-ethylpyrimidin-2-yl)oxy)-3-fluorophenyl)-1-methyl-1H-pyrrolo[3,2-c]pyridin-2-yl)-3-fluorophenyl)methacrylamide;
N-(4-(4-amino-7-cyano-3-(3-fluoro-4-((6-methylpyridin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-(pyridin-2-yloxy)phenyl)-1-methyl-1H-pyrrolo[3,2-c]pyridin-2-yl)-3-fluorophenyl)methacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-fluorophenyl)methacrylamide;
N-(4-(4-amino-7-cyano-1-methyl-3-(4-((6-methylpyridin-2-yl)oxy)phenyl)-1H-pyrrolo[3,2-c]pyridin-2-yl)-3-fluorophenyl)methacrylamide;
N-(4-(4-amino-7-cyano-1-methyl-3-(4-(pyrimidin-2-yloxy)phenyl)-1H-pyrrolo[3,2-c]pyridin-2-yl)-3-fluorophenyl)methacrylamide;
4-(4-amino-7-cyano-2-(2-fluoro-4-methacrylamidophenyl)-1-methyl-1H-pyrrolo[3,2-c]pyridin-3-yl)-2-methoxy-N-(2,2,2-trifluoroethyl)benzamide;
N-(4-(4-amino-3-(3-chloro-4-((4-methylpyrimidin-2-yl)oxy)phenyl)-7-cyano-1-methyl-1H-pyrrolo[3,2-c]pyridin-2-yl)-3-fluorophenyl)methacrylamide;
N-(4-(4-amino-7-cyano-1-methyl-3-(3-methyl-4-((4-methylpyrimidin-2-yl)oxy)phenyl)-1H-pyrrolo[3,2-c]pyridin-2-yl)-3-fluorophenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-chlorophenyl)methacrylamide;
N-(4-(4-amino-7-cyano-3-(3,5-difluoro-4-((4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(4-((4,5-dimethylpyrimidin-2-yl)oxy)-3-fluorophenyl)-1-methyl-1H-pyrrolo[3,2-c]pyridin-2-yl)-3-fluorophenyl)methacrylamide;
N-(4-(4-amino-7-cyano-3-(3-fluoro-4-((5-fluoropyrimidin-2-yl)oxy)phenyl)-1-methyl-1H-pyrrolo[3,2-c]pyridin-2-yl)phenyl)acrylamide;
N-(4-(4-amino-7-cyano-3-(3-fluoro-4-(pyridin-2-yloxy)phenyl)-1-methyl-1H-pyrrolo[3,2-c]pyridin-2-yl)phenyl)acrylamide;
N-(4-(4-amino-7-cyano-3-(3-fluoro-4-((5-fluoropyridin-2-yl)oxy)phenyl)-1-methyl-1H-pyrrolo[3,2-c]pyridin-2-yl)phenyl)acrylamide;
N-(4-(4-amino-7-cyano-3-(4-((4,5-dimethylpyrimidin-2-yl)oxy)-3-fluorophenyl)-1-methyl-1H-pyrrolo[3,2-c]pyridin-2-yl)phenyl)acrylamide;
N-(4-(4-amino-7-cyano-3-(4-(cyclohexyloxy)phenyl)-1-methyl-1H-pyrrolo[3,2-c]pyridin-2-yl)-3-fluorophenyl)methacrylamide;
4-(4-amino-7-cyano-2-(2-fluoro-4-methacrylamidophenyl)-1-methyl-1H-pyrrolo[3,2-c]pyridin-3-yl)-2-methoxy-N-(1-(trifluoromethyl)cyclopropyl)benzamide;
N-(4-(4-amino-7-cyano-1-methyl-3-(4-(pyridin-2-yloxy)phenyl)-1H-pyrrolo[3,2-c]pyridin-2-yl)-3-fluorophenyl)methacrylamide;
N-(4-(4-amino-7-cyano-1-methyl-3-(4-(pyridin-2-yloxy)phenyl)-1H-pyrrolo[3,2-c]pyridin-2-yl)phenyl)acrylamide;
N-(4-(4-amino-7-cyano-3-(3-fluoro-4-((5-fluoropyrimidin-2-yl)oxy)phenyl)-1-methyl-1H-pyrrolo[3,2-c]pyridin-2-yl)-3-fluorophenyl)methacrylamide;
N-(4-(4-amino-3-(3-chloro-4-((5-fluoro-4-methylpyrimidin-2-yl)oxy)phenyl)-7-cyano-1-methyl-1H-pyrrolo[3,2-c]pyridin-2-yl)phenyl)acrylamide;
N-(4-(4-amino-3-(4-((6-chloropyridin-2-yl)oxy)-3-fluorophenyl)-7-cyano-1-methyl-1H-pyrrolo[3,2-c]pyridin-2-yl)-3-fluorophenyl)methacrylamide;
N-(4-(4-amino-7-cyano-3-(2,5-difluoro-4-((4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(2,3-difluoro-4-((4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-2-fluoro-3-methylphenyl)methacrylamide;
N-(4-(4-amino-7-cyano-3-(2-fluoro-4-((4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-((5-fluoropyridin-2-yl)oxy)phenyl)-1-methyl-1H-pyrrolo[3,2-c]pyridin-2-yl)-3-fluorophenyl)methacrylamide;
N-(4-(4-amino-7-cyano-3-(4-((5-fluoropyridin-2-yl)oxy)phenyl)-1-methyl-1H-pyrrolo[3,2-c]pyridin-2-yl)phenyl)acrylamide;
N-(4-(4-amino-7-cyano-3-(4-((5-fluoro-4-methylpyrimidin-2-yl)oxy)-3-methylphenyl)-1-methyl-1H-pyrrolo[3,2-c]pyridin-2-yl)phenyl)acrylamide;
N-(4-(4-amino-7-cyano-3-(3,5-difluoro-4-((5-fluoro-4-methylpyrimidin-2-yl)oxy)phenyl)-1-methyl-1H-pyrrolo[3,2-c]pyridin-2-yl)phenyl)acrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-fluorophenyl)acrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-fluorophenyl)methacrylamide;
N-(4-(4-amino-7-cyano-3-(4-((5-fluoropyridin-2-yl)oxy)phenyl)-1-methyl-1H-pyrrolo[3,2-c]pyridin-2-yl)-3-fluorophenyl)methacrylamide;
N-(4-(4-amino-7-cyano-3-(2,3-difluoro-4-((5-fluoro-4-methylpyrimidin-2-yl)oxy)phenyl)-1-methyl-1H-pyrrolo[3,2-c]pyridin-2-yl)phenyl)acrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-methylphenyl)acrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-chlorophenyl)acrylamide;
N-(4-(4-amino-7-cyano-3-(4-(cyclohexyloxy)phenyl)-1-methyl-1H-pyrrolo[3,2-c]pyridin-2-yl)phenyl)acrylamide;
N-(4-(4-amino-7-cyano-3-(4-(cyclohexanecarbonyl)-3-fluorophenyl)-1-methyl-1H-pyrrolo[3,2-c]pyridin-2-yl)phenyl)acrylamide;
N-(4-(4-amino-7-cyano-3-(3-fluoro-4-(thiazol-2-yloxy)phenyl)-1-methyl-1H-pyrrolo[3,2-c]pyridin-2-yl)phenyl)acrylamide;
N-(4-(4-amino-7-cyano-3-(3-fluoro-4-((4-methylthiazol-2-yl)oxy)phenyl)-1-methyl-1H-pyrrolo[3,2-c]pyridin-2-yl)phenyl)acrylamide;
N-(4-(4-amino-7-cyano-3-(3-fluoro-4-((4-(trifluoromethyl)pyrimidin-2-yl)oxy)phenyl)-1-methyl-1H-pyrrolo[3,2-c]pyridin-2-yl)phenyl)acrylamide;
N-(4-(4-amino-7-cyano-3-(3-fluoro-4-((4-(trifluoromethyl)pyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-2,3-difluorophenyl)methacrylamide;
N-(4-(4-amino-7-cyano-3-(3-fluoro-4-((5-fluoro-6-methylpyridin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-((5-fluoro-6-methylpyridin-2-yl)oxy)phenyl)-1-methyl-1H-pyrrolo[3,2-c]pyridin-2-yl)phenyl)acrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-2,3-difluorophenyl)acrylamide;
N-(4-(4-amino-7-cyano-3-(4-(cyclohexyloxy)-3-fluorophenyl)-1-methyl-1H-pyrrolo[3,2-c]pyridin-2-yl)-3-fluorophenyl)methacrylamide;
N-(4-(4-amino-7-cyano-3-(4-((1,5-dimethyl-1H-pyrazol-3-yl)oxy)-3-fluorophenyl)-1-methyl-1H-pyrrolo[3,2-c]pyridin-2-yl)phenyl)acrylamide;
N-(4-(4-amino-7-cyano-3-(3-fluoro-4-((1-methyl-5-(trifluoromethyl)-1H-pyrazol-3-yl)oxy)phenyl)-1-methyl-1H-pyrrolo[3,2-c]pyridin-2-yl)phenyl)acrylamide;
N-(4-(4-amino-7-cyano-1-methyl-3-(4-((1-methyl-5-(trifluoromethyl)-1H-pyrazol-3-yl)oxy)phenyl)-1H-pyrrolo[3,2-c]pyridin-2-yl)phenyl)acrylamide;
N-(4-(4-amino-7-cyano-1-methyl-3-(4-((4-(trifluoromethyl)pyrimidin-2-yl)oxy)phenyl)-1H-pyrrolo[3,2-c]pyridin-2-yl)phenyl)acrylamide;
N-(4-(4-amino-7-cyano-1-methyl-3-(4-((4-(trifluoromethyl)pyrimidin-2-yl)oxy)phenyl)-1H-pyrrolo[3,2-c]pyridin-2-yl)-3-fluorophenyl)methacrylamide;
N-(4-(4-amino-7-cyano-3-(4-((4,6-dimethylpyrimidin-2-yl)oxy)phenyl)-1-methyl-1H-pyrrolo[3,2-c]pyridin-2-yl)phenyl)acrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-chlorophenyl)methacrylamide;
N-(4-(4-amino-7-cyano-1-methyl-3-(4-((1-methyl-1H-pyrazol-5-yl)oxy)phenyl)-1H-pyrrolo[3,2-c]pyridin-2-yl)phenyl)acrylamide;
N-(4-(4-amino-7-cyano-1-methyl-3-(3-methyl-4-((4-methylpyrimidin-2-yl)oxy)phenyl)-1H-pyrrolo[3,2-c]pyridin-2-yl)-3-chlorophenyl)methacrylamide;
N-(4-(4-amino-7-cyano-3-(4-((1-cyclopropyl-1H-pyrazol-3-yl)oxy)-3-fluorophenyl)-1-methyl-1H-pyrrolo[3,2-c]pyridin-2-yl)phenyl)acrylamide;
N-(4-(4-amino-3-(3-chloro-4-((4-methylpyrimidin-2-yl)oxy)phenyl)-7-cyano-1-methyl-1H-pyrrolo[3,2-c]pyridin-2-yl)-3-chlorophenyl)methacrylamide;
N-(4-(4-amino-7-cyano-1-methyl-3-(4-((6-methylpyridin-2-yl)oxy)phenyl)-1H-pyrrolo[3,2-c]pyridin-2-yl)-3-chlorophenyl)methacrylamide;
4-(3-acrylamido-12-amino-9-cyano-6,7-dihydrobenzo[f]pyrido[3′,4′:4,5]pyrrolo[1,2-d][1,4]oxazepin-13-yl)-N-(3,3-difluorocyclobutyl)-2-methoxybenzamide;
N-(4-(4-amino-3-(3-chloro-4-((4-methylpyrimidin-2-yl)oxy)phenyl)-7-cyano-1-methyl-1H-pyrrolo[3,2-c]pyridin-2-yl)-3-methylphenyl)methacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-chlorophenyl)methacrylamide;
N-(4-(4-amino-7-cyano-3-(3-fluoro-4-((6-methylpyridin-2-yl)oxy)phenyl)-1-methyl-1H-pyrrolo[3,2-c]pyridin-2-yl)-3-chlorophenyl)methacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-fluorophenyl)acrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-fluorophenyl)acrylamide;
N-(4-(4-amino-7-cyano-3-(4-(cyclohexyloxy)phenyl)-1-methyl-1H-pyrrolo[3,2-c]pyridin-2-yl)-3-chlorophenyl)methacrylamide;
N-(4-(4-amino-5-(4-(cyclohexyloxy)-3-fluorophenyl)pyrazolo[5,1-f][1,2,4]triazin-6-yl)phenyl)-2-fluoroacrylamide;
N-(4-(4-amino-5-(4-(cyclohexyloxy)-3-fluorophenyl)pyrazolo[5,1-f][1,2,4]triazin-6-yl)phenyl)acrylamide;
N-(4-(4-amino-7-cyano-3-(4-(cyclohexyloxy)-3-fluorophenyl)-1-methyl-1H-pyrrolo[3,2-c]pyridin-2-yl)phenyl)acrylamide;
N-(4-(4-amino-5-(3-methoxy-4-((4-methylpyrimidin-2-yl)oxy)phenyl)pyrazolo[5,1-f][1,2,4]triazin-6-yl)phenyl)acrylamide;
4-(2-(4-acrylamidophenyl)-4-amino-7-cyano-1-methyl-1H-pyrrolo[3,2-c]pyridin-3-yl)-2-methoxy-N-(2,2,2-trifluoroethyl)benzamide;
4-(4-amino-6-(4-(2-fluoroacrylamido)phenyl) pyrazolo[5,1-f][1,2,4]triazin-5-yl)-2-methoxy-N-(2,2,2-trifluoroethyl)benzamide;
4-(4-amino-7-cyano-2-(4-(2-fluoroacrylamido)phenyl)-1-methyl-1H-pyrrolo[3,2-c]pyridine-3-yl)-2-methoxy-N-(2,2,2-trifluoroethyl)benzamide;
4-(4-amino-6-(2-fluoro-4-(2-fluoroacrylamido)phenyl)pyrazolo[5,1-f][1,2,4]triazin-5-yl)-2-methoxy-N-(2,2,2-trifluoroethyl)benzamide;
4-(4-amino-6-(4-(2-fluoroacrylamido)phenyl)pyrazolo[5,1-f][1,2,4]triazin-5-yl)-N-(3,3-difluorocyclobutyl)benzamide;
N-(4-(6-(4-acrylamidophenyl)-4-aminopyrazolo[5,1-f][1,2,4]triazin-5-yl)-2-methoxyphenyl)-2,2-difluorocyclopropane-1-carboxamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)phenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)phenyl)-2-fluoroacrylamide;
N-(4-(4-amino-5-(3-fluoro-4-((4-methylpyrimidin-2-yl)oxy)phenyl)pyrazolo[5,1-f][1,2,4]triazin-6-yl)phenyl)methacrylamide;
4-(2-(4-acrylamidophenyl)-4-amino-7-cyano-1-(methyl-d3)-1H-pyrrolo[3,2-c]pyridin-3-yl)-2-methoxy-N-(2,2,2-trifluoroethyl)benzamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-chlorophenyl)methacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-methylphenyl)methacrylamide;
N-(4-(4-amino-7-cyano-3-(3-fluoro-4-((1-methyl-1H-pyrazol-3-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-methylphenyl)methacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-chlorophenyl)methacrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)phenyl)acrylamide;
4-(3-acrylamido-12-amino-9-cyano-6,7-dihydrobenzo[f]pyrido[3′,4′:4,5]pyrrolo[1,2-d][1,4]oxazepin-13-yl)-N-(3,3-difluorocyclobutyl)-2-methoxybenzamide;
N-(4-(4-amino-7-cyano-3-(3-fluoro-4-((6-methylpyridin-2-yl)oxy)phenyl)-1-methyl-1H-pyrrolo[3,2-c]pyridin-2-yl)-3-methylphenyl)methacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-methylphenyl)methacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-methylphenyl)methacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-methylphenyl)methacrylamide;
N-(4-(4-amino-7-cyano-1-methyl-3-(3-methyl-4-((4-methylpyrimidin-2-yl)oxy)phenyl)-1H-pyrrolo[3,2-c]pyridin-2-yl)-3-methylphenyl)methacrylamide;
N-(4-(4-amino-7-cyano-1-methyl-3-(4-((6-methylpyridin-2-yl)oxy)phenyl)-1H-pyrrolo[3,2-c]pyridin-2-yl)-3-methylphenyl)methacrylamide;
N-(4-(4-amino-7-cyano-3-(4-((4-(difluoromethyl)pyrimidin-2-yl)oxy)-3-fluorophenyl)-1-methyl-1H-pyrrolo[3,2-c]pyridin-2-yl)-3-chlorophenyl)methacrylamide;
N-(4-(4-amino-7-cyano-3-(3-fluoro-4-((5-fluoro-4-methylpyrimidin-2-yl)oxy)phenyl)-1-methyl-1H-pyrrolo[3,2-c]pyridin-2-yl)-3-chlorophenyl)methacrylamide;
N-(4-(4-amino-7-cyano-3-(3-fluoro-4-((4-(trifluoromethyl)pyrimidin-2-yl)oxy)phenyl)-1-methyl-1H-pyrrolo[3,2-c]pyridin-2-yl)-3-chlorophenyl)methacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-methylphenyl)-2-fluoroacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-methylphenyl)-2-fluoroacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-methylphenyl)-2-fluoroacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-methylphenyl)-2-fluoroacrylamide;
N-(4-(4-amino-7-cyano-1-methyl-3-(4-((6-methylpyridin-2-yl)oxy)phenyl)-1H-pyrrolo[3,2-c]pyridin-2-yl)-3-methylphenyl)-2-fluoroacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-chlorophenyl)-2-fluoroacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-chlorophenyl)-2-fluoroacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-chlorophenyl)-2-fluoroacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-chlorophenyl)-2-fluoroacrylamide;
N-(4-(4-amino-7-cyano-1-methyl-3-(4-((6-methylpyridin-2-yl)oxy)phenyl)-1H-pyrrolo[3,2-c]pyridin-2-yl)-3-chlorophenyl)-2-fluoroacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-fluorophenyl)-2-fluoroacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-fluorophenyl)-2-fluoroacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-fluorophenyl)-2-fluoroacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-fluorophenyl)-2-fluoroacrylamide;
N-(4-(4-amino-7-cyano-1-methyl-3-(4-((6-methylpyridin-2-yl)oxy)phenyl)-1H-pyrrolo[3,2-c]pyridin-2-yl)-3-fluorophenyl)-2-fluoroacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-methylphenyl)acrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-methylphenyl)acrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-methylphenyl)acrylamide;
N-(4-(4-amino-7-cyano-1-methyl-3-(4-((6-methylpyridin-2-yl)oxy)phenyl)-1H-pyrrolo[3,2-c]pyridin-2-yl)-3-methylphenyl)acrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-chlorophenyl)acrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-chlorophenyl)acrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-chlorophenyl)acrylamide;
N-(4-(4-amino-7-cyano-1-methyl-3-(4-((6-methylpyridin-2-yl)oxy)phenyl)-1H-pyrrolo[3,2-c]pyridin-2-yl)-3-chlorophenyl)acrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-fluorophenyl)acrylamide;
N-(4-(4-amino-7-cyano-1-methyl-3-(4-((6-methylpyridin-2-yl)oxy)phenyl)-1H-pyrrolo[3,2-c]pyridin-2-yl)-3-fluorophenyl)acrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)phenyl)-2-methylprop-2-enethioamide;
N-(4-(4-amino-7-cyano-3-(4-((4-methylpyrimidin-2-yl)oxy)phenyl)-1H-pyrrolo[3,2-c]pyridin-2-yl)-3-chlorophenyl)methacrylamide;
N-(4-(4-amino-7-cyano-1-ethyl-3-(4-((4-methylpyrimidin-2-yl)oxy)phenyl)-1H-pyrrolo[3,2-c]pyridin-2-yl)-3-chlorophenyl)methacrylamide;
N-(4-(4-amino-7-cyano-1-(2-fluoroethyl)-3-(4-((4-methylpyrimidin-2-yl)oxy)phenyl)-1H-pyrrolo[3,2-c]pyridin-2-yl)-3-chlorophenyl)methacrylamide;
N-(4-(4-amino-7-cyano-3-(4-((4-methylpyrimidin-2-yl)oxy)phenyl)-1-(2,2,2-trifluoroethyl)-1H-pyrrolo[3,2-c]pyridin-2-yl)-3-chlorophenyl)methacrylamide;
N-(4-(4-amino-7-cyano-3-(4-((4-(difluoromethyl)-5-fluoropyrimidin-2-yl)oxy)phenyl)-1-methyl-1H-pyrrolo[3,2-c]pyridin-2-yl)phenyl)acrylamide;
N-(4-(4-amino-7-cyano-3-(4-((4-(difluoromethyl)-5-fluoropyrimidin-2-yl)oxy)-3-fluorophenyl)-1-methyl-1H-pyrrolo[3,2-c]pyridin-2-yl)phenyl)acrylamide;
N-(4-(4-amino-3-(3-chloro-5-fluoro-4-((5-fluoro-4-methylpyrimidin-2-yl)oxy)phenyl)-7-cyano-1-methyl-1H-pyrrolo[3,2-c]pyridin-2-yl)phenyl)acrylamide;
N-(4-(4-amino-7-cyano-3-(3-fluoro-4-((1-(2,2,2-trifluoroethyl)-1H-pyrazol-3-yl)oxy)phenyl)-1-methyl-1H-pyrrolo[3,2-c]pyridin-2-yl)phenyl)acrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3,5-difluorophenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-fluoro-5-methylphenyl)methacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-(trifluoromethyl)phenyl)methacrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)phenyl)acrylamide;
N-(4-(4-amino-3-(4-((5-chloro-4-methylpyrimidin-2-yl)oxy)-3,5-difluorophenyl)-7-cyano-1-methyl-1H-pyrrolo[3,2-c]pyridin-2-yl)phenyl)acrylamide;
N-(4-(4-amino-3-(4-((5-chloro-4-(trifluoromethyl)pyrimidin-2-yl)oxy)phenyl)-7-cyano-1-methyl-1H-pyrrolo[3,2-c]pyridin-2-yl)phenyl)acrylamide;
N-(4-(4-amino-3-(4-((5-chloro-4-(trifluoromethyl)pyrimidin-2-yl)oxy)-3-fluorophenyl)-7-cyano-1-methyl-1H-pyrrolo[3,2-c]pyridin-2-yl)phenyl)acrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-chlorophenyl)methacrylamide;
N-(4-(4-amino-7-cyano-1-methyl-3-(4-((4-(trifluoromethyl)pyrimidin-2-yl)oxy)phenyl)-1H-pyrrolo[3,2-c]pyridin-2-yl)-3-chlorophenyl)methacrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-methylphenyl)methacrylamide;
N-(4-(4-amino-7-cyano-1-methyl-3-(4-((4-(trifluoromethyl)pyrimidin-2-yl)oxy)phenyl)-1H-pyrrolo[3,2-c]pyridin-2-yl)-3-methylphenyl)methacrylamide;
N-(4-(4-amino-7-cyano-3-(3-fluoro-4-((4-(trifluoromethyl)pyrimidin-2-yl)oxy)phenyl)-1-methyl-1H-pyrrolo[3,2-c]pyridin-2-yl)-3-methylphenyl)methacrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)phenyl)but-2-ynamide;
N-(4-(4-amino-3-(4-((5-chloro-4-(difluoromethyl)pyrimidin-2-yl)oxy)phenyl)-7-cyano-1-methyl-1H-pyrrolo[3,2-c]pyridin-2-yl)phenyl)acrylamide;
N-(4-(4-amino-3-(4-((5-chloro-4-(difluoromethyl)pyrimidin-2-yl)oxy)-3-fluorophenyl)-7-cyano-1-methyl-1H-pyrrolo[3,2-c]pyridin-2-yl)phenyl)acrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)phenyl)-2-fluoroacrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)phenyl)-2-fluoroacrylamide;
N-(4-(4-amino-3-(4-((5-chloro-6-methylpyridin-2-yl)oxy)phenyl)-7-cyano-1-methyl-1H-pyrrolo[3,2-c]pyridin-2-yl)phenyl)acrylamide;
N-(4-(4-amino-3-(4-((5-chloro-6-methylpyridin-2-yl)oxy)-3-fluorophenyl)-7-cyano-1-methyl-1H-pyrrolo[3,2-c]pyridin-2-yl)phenyl)acrylamide;
N-(4-(4-amino-3-(3-chloro-4-((5-chloro-4-methylpyrimidin-2-yl)oxy)phenyl)-7-cyano-1-methyl-1H-pyrrolo[3,2-c]pyridin-2-yl)phenyl)acrylamide;
N-(4-(4-amino-3-(4-((5-chloro-4-methylpyrimidin-2-yl)oxy)-3-methylphenyl)-7-cyano-1-methyl-1H-pyrrolo[3,2-c]pyridin-2-yl)phenyl)acrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-chlorophenyl)methacrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-methylphenyl)acrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-methylphenyl)acrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-methylphenyl)-2-fluoroacrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-methylphenyl)-2-fluoroacrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-chlorophenyl)acrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-chlorophenyl)acrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-chlorophenyl)-2-fluoroacrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-chlorophenyl)-2-fluoroacrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-fluorophenyl)acrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-fluorophenyl)acrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-fluorophenyl)-2-fluoroacrylamide; or
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-fluorophenyl)-2-fluoroacrylamide.

The present invention furthermore provides a pharmaceutical composition, comprising at least one of compound of Formula (I), and a pharmaceutically acceptable carrier.

In some embodiments of pharmaceutical composition comprising compound of Formula (I) provided by the present invention, the pharmaceutical composition, comprise a compound of Examples, and a pharmaceutically acceptable carrier.

In some embodiments of pharmaceutical composition comprising compound of Formula (I) provided by the present invention, the pharmaceutical composition, comprise a compound of Table 1, and a pharmaceutically acceptable carrier.

In some embodiments of pharmaceutical composition comprising compound of Formula (I) provided by the present invention, the pharmaceutical composition, comprise a compound of Table 2, and a pharmaceutically acceptable carrier.

The present invention furthermore provides a method of inhibiting FGFR2 signaling activity in a subject, comprising administering a therapeutically effective amount of a compound of Formula (I) and the pharmaceutical composition thereof, to a subject in need thereof.

The present invention furthermore provides a method of treating an FGFR2-mediated disorder in a subject, comprising administering a therapeutically effective amount of a compound of Formula (I) and the pharmaceutical composition thereof, to a subject in need thereof.

The present invention furthermore provides a method of treating a disorder in a subject, comprising administering a therapeutically effective amount of a compound of Formula (I) and the pharmaceutical composition thereof, to a subject in need thereof, wherein, the disorder is bile duct cancer, liver cancer, breast cancer, prostate cancer, lung cancer, thyroid cancer, gastric cancer, ovarian cancer, rectal cancer, endometrial cancer, or urothelial cancer.

In some embodiments of the method of treating a disorder in a subject, the disorder is bile duct cancer.

In some embodiments of the method of treating a disorder in a subject, the bile duct cancer is intrahepatic cholangiocarcinoma.

In some embodiments of the method of treating a disorder in a subject, the disorder is liver cancer.

In some embodiments of the method of treating a disorder in a subject, the liver cancer is hepatocellular carcinoma.

In some embodiments of the method of treating a disorder in a subject, the disorder is lung cancer.

In some embodiments of the method of treating a disorder in a subject, the lung cancer is lung squamous cell carcinoma or non-small cell lung cancer.

Representative compounds in the compounds of the above Formula I are shown in Examples, Table 1 and Table 2.

The compounds provided by the present invention are highly potent against kinase FGFR-2 in both enzyme and cellular assays, and are highly selective against same farmily kinases FGFR-1 and FGFR-3. Many compounds in the invention demonstrate good to excellent exposure in rats PK experiments.

### Definitions

The following description is made with the understanding that the present disclosure is to be considered as an exemplification of the claimed subject matter, and is not intended to limit the appended claims to the specific embodiments illustrated. The headings used in this disclosure are provided for convenience and are not to be considered to limit the claims in any way. Embodiments illustrated under any heading might be combined with any other illustrated heading embodiments.

All technical and scientific terms used herein, unless defined otherwise, have the same meaning as commonly understood by one of ordinary skill in the art. It must be noted that as used in this disclosure, the singular forms “a”, “and”, and “the” include plural referents unless the context clearly dictates otherwise. Thus, “the compound” includes a plurality of such compounds and “the assay” includes one or more assays, and so forth.

As used in the present disclosure, the following words, phrases and symbols are generally intended to have the meanings as set forth below, except to the extent that the context in which they are used indicates otherwise.

A dash “-” unless otherwise indicated, as used herein, at the front or end of a chemical group is used, a matter of convenience, to indicate a point of attachment for a substituent. For example, —OH is attached through the carbon atom; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. A solid line coming out of the center of a ring indicates that the point of attachment for a substituent on the ring can be at any ring atom.

The prefix “Cₘ₋ₙ” unless otherwise indicated, as used herein, indicates that the following group has from m to n carbon atoms. For example, “C₁₋₆ alkyl” indicates that the alkyl group has from 1 to 6 carbon atoms. In similar manner, the term “m-n membered” rings, wherein m and n means integer ranges from m to n, such as “3-7 membered heterocyclic ring”, refers to a ring containing 3-7 atoms, of which up to 80% may be heteroatoms, such as N, O, or S, and the remaining atoms are carbon.

The term “aliphatic” or “aliphatic group”, unless otherwise indicated, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic ring”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Also, some commonly used alternative chemical names may or may not be used. For example, a divalent group such as a divalent “alkyl” group, a divalent “aryl” group, etc., may also be referred to as an “alkylene” group or an “alkylenyl” group, or alkylyl group, an “arylene” group, an “arylenyl” group, or arylyl group, a “phenyl” group or “phenylene” group, respectively.

“A compound provided herein” or “a compound described herein” or “a compound disclosed herein” or “a compound of the present disclosure” refers to the compounds of Formula I, II, III, IV, V, VI, VII, I-I, or I-II.

In certain embodiments, the term “about”, directed to that value or parameter per se, includes the indicated amount ±10%, ±5%, or ±1%. Also, the term “about X” includes description of “X”.

“Adjoining atoms”, as used herein, refers to atoms that are in immediately next to each other. For instance, in “C1-C2-C3-C4” atom C1 is adjoining to atom C2, atom C2 is adjoining to atoms C1 and C3, so on and so forth.

“Optional” or “optionally” means that the subsequently described event or circumstance may or may not occur, and that the description includes instances where the said event or circumstance occurs and instances in which it does not. Also, the term “optionally substituted” means that any one or more hydrogen atoms on the designated atom or group may or may not be replaced by a moiety other than hydrogen.

The term “substituted” means that one or more hydrogen atoms on the designated atom or group is substituted with one or more substituents other than hydrogen, in the conditions that the designated atom's normal valence is not exceeded. For example, one or more hydrogen of alkyl, alkylene, alkenyl, alknyl, OH, or NH2 and so on can be substituted with one or more substituents. The substituents include, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. Similar indefinite structures arrived at by defining substituents with further substituents appended to infinity (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl) substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. Whenever used to modify a chemical group, “substituted” may describe other chemical groups defined herein. For example, the term “substituted aryl” includes, but not limited to, “alkylaryl.” Unless specified otherwise, if a group is described as optionally substituted, any substituents of the group are themselves unsubstituted.

As used herein, the term “C₁₋₆ (or C₁₋₄, or C₁₋₃) bivalent saturated or unsaturated, straight or branched, hydrocarbon chain”, refers to bivalent alkylene, alkenylene, and alkynylene chains that are straight or branched as defined herein.

The term “alkylene”, unless otherwise indicated, as used herein, refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene”, unless otherwise indicated, as used herein, refers to a bivalent alkenyl group. A substituted alkenylene chain is a polymethylene group containing at least one double bond in which one or more hydrogen atoms are replaced with a substituent. Suitable substituents include those described below for a substituted aliphaticgroup.

The terms “alkylene”, “arylene”, “cycloalkylene”, “heteroarylene”, “heterocycloalkylene”, and the other similar terms with the suffix “-ylene”, unless otherwise indicated, as used herein, refers to a divalently bonded version of the group that the suffix modifies. For example, “alkylene” is a divalent alkyl group connecting the groups to which it is attached.

The term “alkyl”, unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₆ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, “butyl” includes n-butyl (i.e. —(CH₂)₃CH₃), sec-butyl (i.e. —CH(CH₃)CH₂CH₃), isobutyl (i.e. —CH₂CH(CH₃)₂) and the like. “propyl” includes n-propyl (i.e —(CH₂)₂CH₃) and isopropyl (i.e. —CH(CH₃)₂), pentyl includes 2-pentyl, isopentyl, or neopentyl.

The term “alkenyl” refers to an aliphatic group containing at least one carbon-carbon double bond (C═C) and having from 2 to 15 carbon atoms (i.e., C₂₋₁₅ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

The term “alkynyl” refers to an aliphatic group containing at least one carbon-carbon triple bond (C═C) and having from 2 to 10 carbon atoms (i.e., C₂₋₁₀ alkynyl), or 2 to 4 carbon atoms (i.e., C₂₋₄ alkynyl), etc. The term “alkynyl” also includes those groups having one triple bond and one double bond.

The term “alkoxy” refers to the group “—O-alkyl”, for example, methoxy, ethoxy, n-propoxy, iso-propoxy, n-butoxy, tert-butoxy, sec-butoxy, n-pentoxy, n-hexoxy, and 1,2-dimethylbutoxy.

The term “haloalkoxy” refers to an alkoxy group as indicated above, wherein one or more hydrogen atoms are replaced by a halogen.

The term “lower alkyl” refers to a C₁₋₄ straight or branched alkyl group. Exemplary lower alkyl groups are methyl, ethyl, propyl, isopropyl, butyl, isobutyl, and tert-butyl

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or a substitutable nitrogen of a heterocyclic ring, for example N (as in 3,4-dihydro-2H-pyrrolyl), NH (as in pyrrolidinyl) or NR+ (as in N-substituted pyrrolidinyl)).

The term “unsaturated,” as used herein, means that a moiety has one or more units of unsaturation.

The term “partially unsaturated” in the context of rings, unless otherwise defined, refers to a monocyclic ring, or a component ring within a polycyclic (e.g. bicyclic, tricyclic, etc.) ring system, wherein the component ring contains at least one degree of unsaturation in addition to those provided by the ring itself, but is not aromatic. Examples of partially unsaturated rings include, but are not limited to, 3,4-dihydro-2H-pyran, 3-pyrroline, 2-thiazoline, etc. Where a partially unsaturated ring is part of a polycyclic ring system, the other component rings in the polycyclic ring system may be saturated, partially unsaturated, or aromatic, but the point of attachment of the polycyclic ring system is on a partially unsaturated component ring. For example, unless otherwise defined, 1,2,3,4-tetrahydroquinoline is a partially unsaturated ring if its point of attachment is through the piperidino ring, e.g:

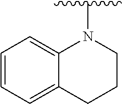 [SMILES: *N1CCCc2ccccc21]

The term “saturated” in the context of rings, unless otherwise defined, refers to a 3-10 membered monocyclic ring, or a 7-14 membered polycyclic (e.g. bicyclic, tricyclic, etc.) ring system, wherein the monocyclic ring or the component ring that is the point of attachment for the polycyclic ring system contains no additional degrees of unsaturation in addition to that provided by the ring itself. Examples of monocyclic saturated rings include, but are not limited to, azetidine, oxetane, cyclohexane, etc. Where a saturated ring is part of a polycyclic ring system, the other component rings in the polycyclic ring system may be saturated, partially unsaturated, or aromatic, but the point of attachment of the polycyclic ring system is on a saturated component ring. For example, unless otherwise defined, 2-azaspiro[3.4]oct-6-ene is a saturated ring if its point of attachment is through the azetidino ring, e.g:

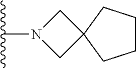 [SMILES: *N1CC2(CCCC2)C1]

As used herein, the term “bridged bicyclic” refers to any bicyclic ring system, i.e carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

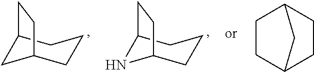 [SMILES: C1CC2CCC(C1)C2 ; C1CC2CCC(C1)N2 ; C1CC2CCC1C2]

The term “acyl” refers to a group —C(═O)R, herein R is hydrogen, alkyl, cycloalkyl, heterocyclyl, aryl, heteroalkyl, or heteroaryl, and each of which may be optionally substituted, as defined herein. Examples of acyl group include formyl, acetyl, cylcohexylcarbonyl, cyclohexylmethyl-carbonyl, and benzoyl, etc.

The term “amido” refers to both a “C-amido” group which refers to the group —C(═O)NR^{a}R^{b} and an “N-amido” group which refers to the group —NR^{a}C(═O)R^{b}, wherein Ra and R^{b} are independently selected from groups consisting of hydrogen, alkyl, aryl, haloalkyl, heteroaryl, cycloalkyl, and heterocyclyl; and each of which may be optionally substituted.

“Amino” refers to the group —NRₐR_{b}, herein Rₐ and R_{b} are independently selected from groups consisting of hydrogen, alkyl, haloalkyl, aryl, heteroaryl, cycloalkyl, and heterocyclyl; and each of which may be optionally substituted.

The term “aryl” refers to an aromatic carbocyclic group having a single ring (e.g. monocyclic) or multiple rings (e.g. bicyclic or tricyclic) including fused systems. As examples used herein, aryl has 6 to 20 ring carbon atoms (i.e., C₆₋₂₀ aryl), 6 to 12 carbon ring atoms (i.e., C₆₋₁₂ aryl), etc. Some examples of aryl groups include phenyl, naphthyl, fluorenyl, and anthryl. Herein, aryl does not encompass or overlap in any way with heteroaryl as defined below. If one or more aryl groups are fused with a heteroaryl ring, the resulting ring system is heteroaryl.

The term “cyano” or “carbonitrile” group represented by —CN

The term “cycloalkyl” refers to a saturated or partially saturated cyclic alkyl group having a single ring or multiple rings including fused, bridged, and spiro ring systems. The term “cycloalkyl” also includes cycloalkenyl groups (i.e. the cyclic group having at least one double bond). As used wherein, cycloalkyl has from 3 to 20 ring carbon atoms (i.e., C₃₋₂₀ cycloalkyl), 3 to 8 ring carbon atoms (i.e., C₃₋₈ cycloalkyl), or 3 to 5 ring carbon atoms (i.e., C₃₋₅ cycloalkyl), etc. Examples include cyclopropyl, cyclobutyl, cyclopentyl, and cyclohexyl.

The term “bridged” refers to a ring fusion wherein non-adjacent atoms on a ring are joined by a divalent substituent, such as alkylenyl group, an alkylenyl group containing one or two heteroatoms, or a single heteroatom. Examples of bridged ring systems include quinuclidinyl and admantanyl.

The term “fused” refers to a ring which is bound to an adjacent ring.

The term “spiro” refers to a ring substituent which is joined by two bonds at the same carbon atom. Some examples of spiro groups include 1,1-diethylcyclopentane, dimethyl-dioxolane, and 4-benzyl-4-methylpiperidine, wherein the cyclopentane and piperidine, respectively, are the spiro substituents.

“Halogen” or “halo” includes fluoro (F), chloro (Cl), bromo (Br), and iodo (I).

“Deuterium”, “D” “deuterium” all refer to deuterium.

“N” maybe represents a nitrogen atom, or a bond containing nitrogen, or —NH, depending on the context. Similarly, “S” maybe represents a sulfur atom or an bond contain sulfur, “O” maybe represents an oxygen atom or an bond contain oxygen.

“Haloalkyl” includes an unbranched or branched alkyl group as defined above, wherein one or more hydrogen atoms are replaced by a halogen. If a residue is substituted with more than one halogen, it may be referred to by using a prefix corresponding to the number of halogen moieties attached. Dihaloalkyl and trihaloalkyl refer to alkyl substituted with two (“di”) or three (“tri”)halo groups, which may be, but are not necessarily, the same halogen. Some examples of haloalkyl include difluoromethyl (—CHF₂) and trifluoromethyl (—CF₃).

The term “heteroaryl” refers to an aromatic group having a single ring, multiple rings, or multiple fused rings, with one or more ring heteroatoms independently selected from N, O, and S. As examples used herein, heteroaryl includes 1 to 20 carbon ring atoms (i.e., C₁₋₂₀ heteroaryl), 3 to 12 carbon ring atoms (i.e., C₃₋₁₂ heteroaryl), etc., and the number of ring heteroatoms, as used wherein, independently selected from N, O, or S. Examples of heteroaryl groups include pyrimidinyl, purinyl, pyridyl, pyridazinyl, benzothiazolyl, and pyrazolyl. The term “heteroaryl” does not encompass or overlap with “aryl” as defined above.

The term “heterocyclyl” or “heterocyclic ring” refers to a non-aromatic cyclic alkyl group, with at least one ring heteroatoms independently selected from boron (B), nitrogen (N), oxygen (O), phosphorus (P) and sulfur. As used wherein, “heterocyclyl” or “heterocyclic ring” refer to rings that are saturated or partially saturated unless otherwise indicated, for example, in some embodiments “heterocyclyl” or “heterocyclic ring” refers to rings that are partially saturated where specified. “Heterocyclyl” or “heterocyclic ring” includes heterocycloalkenyl groups (i.e., the heterocyclyl group having at least one double bond). A heterocyclyl may be a single ring or multiple rings wherein the multiple rings may be fused, bridged, or spiro. As used herein, heterocyclyl has 2 to 20 carbon ring atoms (i.e., C₂₋₂₀ heterocyclyl), 3 to 8 carbon ring atoms (i.e., C₃₋₈ heterocyclyl), or 3 to 6 carbon ring atoms (i.e., C₃₋₆ heterocyclyl); having 1 to 5 ring heteroatoms independently selected from B, N, O, P, or S. Examples of heterocyclyl groups include pyrrolidinyl, piperidinyl, piperazinyl, oxetanyl, dioxolanyl, azetidinyl, and morpholinyl.

The term “bridged-heterocyclyl” refers to a four- to ten-membered cyclic moiety connected at two non-adjacent atoms of the heterocyclyl with one or more (e.g., 1 or 2) four- to ten-membered cyclic moiety having at least one heteroatom where each heteroatom is independently selected from B, N, O, P and S. As used herein, “bridged-heterocyclyl” includes bicyclic and tricyclic ring systems. Also as used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Also as used herein, the term “fused-heterocyclyl” refers to a three- to ten-membered cyclic moiety connected at two adjacent atoms of the heterocyclyl with one or more (e.g., 1 or 2) three- to ten-membered cyclic moiety having at least one heteroatom where each heteroatom is independently selected from B, N, O, P and S. As used herein, “fused-heterocyclyl” includes bicyclic and tricyclic ring systems. As used herein, the terms “heterocyclyl”, and “heterocyclic ring” are used interchangeably, and include mono-, bridged-, spiro-, or fused-carbocyclic ring and the combinations thereof. In some embodiments, a heterocyclyl is substituted with an oxo group.

The term “carbocyclyl” or “carbocyclic ring” refers to a non-aromatic cyclic aliphatic group, without any ring heteroatoms, wherein, “carbocyclyl” or “carbocyclic ring” refer to rings that are saturated or partially saturated unless otherwise indicated, for example, in some embodiments “carbocyclyl” or “carbocyclic ring” refers to rings that are partially saturated where specified. “Carbocyclyl” or “carbocyclic ring” includes carbocycloalkenyl groups (i.e., the carbocyclic group having at least one double bond). A carbocyclic ring may be a single ring or multiple rings wherein the multiple rings may be fused, bridged, or spiro. As used herein, carbocyclic ring has 3 to 14 carbon ring atoms (i.e., C₃₋₁₄ carbocyclic ring or C₃₋₁₄ carbocyclic ring), 3 to 8 carbon ring atoms (i.e., C₃-s carbocyclic ring), or 3 to 6 carbon ring atoms (i.e., C₃₋₆ carbocyclic ring); Examples of carbocyclic groups include cyclopropyl, cyclobutyl, cyclopentyl, cyclohexyl. The term “bridged-carboncyclic ring” refers to a three- to ten-membered cyclic moiety connected at two non-adjacent carbon atoms of the carbocyclyl with one or more (e.g., 1 or 2) three- to ten-membered cyclic moiety. As used herein, “bridged-carbocyclyl” or “bridged-carbocyclic ring” includes bicyclic and tricyclic ring systems. Also as used herein, the term “spiro-carbocyclyl” or “spiro-carbocyclic ring” includes bicyclic and tricyclic ring systems, refers to a ring system in which a three- to ten-membered carbocyclyl has one or more additional ring, wherein the one or more additional ring is three- to ten-membered carbocyclyl, where a single atom of the one or more additional ring is also an atom of the three- to ten-membered carbocyclyl. Also as used herein, the term “fused-carbocyclyl” or “fused-carbocyclic ring” includes bicyclic and tricyclic ring systems, refers to a three- to ten-membered cyclic moiety connected at two adjacent carbon atoms of the carbocyclyl with one or more (e.g., 1 or 2) three- to ten-membered cyclic moiety. As used herein, the terms “carbocyclyl”, and “carbocyclic ring” are used interchangeably, and include mono-, bridged-, spiro-, or fused-carbocyclic ring and the combinations thereof.

“Hydroxy” or “hydroxyl” refers to the group —OH.

“Oxo” refers to the group (═O) or (O).

The term “sulfonyl” refers to the group —S(O)₂R^{a}, where R^{a} is alkyl, haloalkyl, heterocyclyl, cycloalkyl, heteroaryl, or aryl. Some Examples of sulfonyl are methylsulfonyl, ethylsulfonyl, phenylsulfonyl, and toluenesulfonyl.

Wherever a group terminates in a singly bonded nitrogen atom, that group represents an —NH group unless otherwise indicated. Similarly, unless otherwise expressed, hydrogen atom(s) are implied and deemed present where necessary in view of the knowledge of one of skill in the art to complete valency or provide stability.

“Optional” or “optionally” means that the subsequently described event or circumstance may or may not occur, and that the description includes instances where the said event or circumstance occurs and instances in which it does not. Also, the term “optionally substituted” means that any one or more hydrogen atoms on the designated atom or group may or may not be replaced by a suitable substituent other than hydrogen. the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

“Substituted” means that one or more hydrogen atoms on the designated atom or group is substituted with one or more substituents other than hydrogen, in the conditions that the designated atom's normal valence is not exceeded. The substituents include, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. Similar indefinite structures arrived at by defining substituents with further substituents appended to infinity (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl) substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. Whenever used to modify a chemical group, “substituted” may describe other chemical groups defined herein. For example, the term “substituted aryl” includes, but not limited to, “alkylaryl.” Unless specified otherwise, if a group is described as optionally substituted, any substituents of the group are themselves unsubstituted.

In some instances, “substituted alkyl” refers to an alkyl group having one or more substituents that include hydroxyl, halo, amino, alkoxy, cycloalkyl, heterocyclyl, aryl, and heteroaryl. In additional instances, “substituted cycloalkyl” refers to a cycloalkyl group having one or more substituents including alkyl, haloalkyl, cycloalkyl, heterocyclyl, aryl, heteroaryl, amino, alkoxy, halo, oxo, and hydroxyl; by “substituted heterocyclyl”, it refers to a heterocyclyl group having one or more substituents including alkyl, amino, haloalkyl, heterocyclyl, cycloalkyl, aryl, heteroaryl, alkoxy, halo, oxo, and hydroxyl; the term “substituted aryl” refers to an aryl group having one or more substituents including halo, alkyl, amino, haloalkyl, cycloalkyl, heterocyclyl, heteroaryl, alkoxy, and cyano; the term “substituted heteroaryl” refers to an heteroaryl group having one or more substituents including halo, amino, alkyl, haloalkyl, cycloalkyl, aryl, heterocyclyl, heteroaryl, alkoxy, and cyano and the term “substituted sulfonyl” refers to a group —S(O)₂R, herein R is substituted with one or more substituents including alkyl, cycloalkyl, heterocyclyl, aryl, and heteroaryl. In other instances, the one or more substituents may be further substituted with halo, alkyl, haloalkyl, hydroxyl, alkoxy, cycloalkyl, heterocyclyl, aryl, or heteroaryl, each of which is substituted. In other instances, the substituents may be further substituted with halo, alkyl, haloalkyl, alkoxy, hydroxyl, cycloalkyl, heterocyclyl, aryl, or heteroaryl, each of which is unsubstituted.

In some embodiments, a substituted cycloalkyl, a substituted heterocyclyl, a substituted aryl, and/or a substituted heteroaryl includes a cycloalkyl, a heterocyclyl, an aryl, and/or a heteroaryl that has a substituent on the ring atom to which the cycloalkyl, heterocyclyl, aryl, and/or heteroaryl is attached to the rest of the compound. For example, in the below moiety, the phenyl ring is substituted with a meta-chloro group:

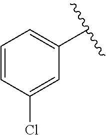 [SMILES: *c1cccc(Cl)c1]

The disclosed compounds herein, or their pharmaceutically acceptable salts may contain one or more asymmetric centers and thus give rise to enantiomers, diastereomers, and other stereoisomeric forms that may be defined, in terms of absolute stereochemistry, as (R)- or (S)- or, as (D)- or (L)- for amino acids. The current disclosure includes all such possible isomers, as well as their racemic and optically pure forms. Optically active (+) and (−), (R)- and (S)-, or (D)- and (L)-isomers may be prepared using chiral synthons or chiral reagents, or resolved by conventional techniques, such as, chromatography and fractional crystallization. Traditional techniques for the preparation, isolation of individual enantiomers include chiral synthesis from a suitable optically pure precursor or resolution of the racemate (or the racemate of a salt or derivative) using, for example, chiral high pressure liquid chromatography (HPLC). When the compounds disclosed herein contain olefinic double bonds or other centers of geometric asymmetry, and unless specified otherwise, these compounds include both E and Z geometric isomers. As the same, all tautomeric forms are also intended to be included. Wherever compounds are represented in their chiral form, it is understood that the embodiment includes, but is not limited to, the specific diastereomerically or enantiomerically enriched form. In situations that the chirality is not specified but is present, it is understood that the embodiment is intended to include either the specific diastereomerically or enantiomerically enriched form; or a racemic or scalemic mixture of such compound(s). “Scalemic mixture” is a mixture of stereoisomers at a ratio other than 1:1.

The term “stereoisomer” refers to a compound containing the same atoms bonded by the same bonds but having different three-dimensional structures, which are not interchangeable. The current disclosure contemplates various stereoisomers and mixtures thereof and includes “enantiomers”, which refers to two stereoisomers whose molecules are non-superimposable mirror images of one another.

The term “enantiomers” represent a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

The term “diastereoisomers” represent stereoisomers that have at least two asymmetric atoms, but which are not mirror-images of each other.

“Tautomer” refers to a proton shift from one atom of a molecule to another atom of the same molecule. The present disclosure includes tautomers of any compounds provided herein.

An “effective amount”, “sufficient amount” or “therapeutically effective amount” as used herein is an amount of a compound that is sufficient to effect beneficial or desired results, including clinical results. As such, the effective amount may be sufficient, e.g., to reduce or ameliorate the severity and/or duration of afflictions related to FGFR2 signaling, or one or more symptoms thereof, prevent the advancement of conditions or symptoms related to afflictions related to FGFR2 signaling, or enhance or otherwise improve the prophylactic or therapeutic effect(s) of another therapy. An effective amount also includes the amount of the compound that avoids or substantially attenuates undesirable side effects

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable “Treatment” can also mean prolonging survival as compared to expected survival if not receiving treatment. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “in need thereof” refers to the need for symptomatic or asymptomatic relief from conditions related to FGFR2 signaling activity or that may otherwise be relieved by the compounds and/or compositions of the disclosure

Some disclosed compounds herein exist as tautomeric isomers. Tautomeric isomers are in equilibrium with one another. For example, amide containing compounds may exist in equilibrium with imidic acid tautomers. No matter which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. As the same, the imidic acid containing compounds are understood to include their amide tautomers.

The interaction of a solvent and a compound forms a “solvate”. Herein, the solvates also include the solvates of salts of the compounds disclosed and the hydrates of the compounds provided herein.

Any formula or structure provided herein also represents unlabeled forms as well as isotopically labeled forms of the compounds. Isotopically labeled compounds have the same structures as depicted by the formulas given herein, except that one or more atoms are replaced by an atom having a selected atomic mass or mass number. Examples of isotopes include isotopes, such as, of carbon (¹¹C, ¹³C, ¹⁴C), nitrogen (¹⁵N), oxygen (¹⁷O, ¹⁸O), phosphorous (³¹P, ³²P), fluorine (¹⁸F), chlorine (³⁶Cl), and iodine (¹²⁵I). Isotopically labelled compounds may have usages in metabolic studies, reaction kinetic studies, detection or imaging techniques, such as positron emission tomography (PET) or single-photon emission computed tomography (SPECT) in drug or substrate tissue distribution assays or in radioactive treatment of patients.

In many cases, the current disclosed compounds are capable to form acid salts by virtue of the presence of amino and/or groups similar thereto.

The term “pharmaceutically acceptable salts” refers to salts prepared from pharmaceutically acceptable non-toxic acids. When the compound of the present invention is basic, its corresponding salt can be conveniently prepared from pharmaceutically acceptable non-toxic acids, including inorganic and organic acids. Since the compounds of Formula I, II, III, IV, V, VI, VII, I-I, or I—II are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I, II, III, IV, V, VI, VII, I-I, or I-II, or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, II, III, IV, V, VI, VII, I-I, or I-II, or a prodrug or a metabolite or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, II, III, IV, V, VI, VII, I-I, or I-II, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt, of Formula I, II, III, IV, V, VI, VII, I-I, or I-II. The compounds of Formula I, II, III, IV, V, VI, VII, I-I, or I-II, or pharmaceutically acceptable salts thereof, can also be included in pharmaceutical compositions in combination with one or more other therapeutically active compounds.

The pharmaceutical carrier employed can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 0.05 to about 95 percent of the total composition. Unit dosage forms will generally contain between from about 0.01 mg to about 2 g of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound represented by Formula I of this invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I or pharmaceutically acceptable salts thereof, may also be prepared in powder or liquid concentrate form.

Generally, dosage levels on the order of from about 0.001 mg/kg to about 150 mg/kg of body weight per day are useful in the treatment of the above-indicated conditions or alternatively about 0.05 mg to about 7 g per patient per day. For example, cancer, disease and conditions of the immune system, may be effectively treated by the administration of from about 0.001 to 50 mg of the compound per kilogram of body weight per day or alternatively about 0.05 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The term “disease” refers to any disease, discomfort, illness, symptoms or indications, and can be interchangeable with the term “disorder” or “condition”.

The term “cancer” encompasses all forms of cancers, including, but not limited to, all forms of carcinomas, melanomas, blastomas, sarcomas, lymphomas and leukemias. Examples include but are not limited to breast cancer, bladder cancer, bladder carcinoma, uterine cancer, brain tumors, cervical cancer, colorectal cancer, esophageal cancer, endometrial cancer, liver cancer(including HCC), laryngeal cancer, lung cancer, osteosarcoma, ovarian cancer, pancreatic cancer, prostate cancer, renal carcinoma, kidney cancer (including RCC), thyroid cancer, acute lymphocytic leukemia, acute myeloid leukemia, ependymoma, Ewing's sarcoma, glioblastoma, medulloblastoma, neuroblastoma, osteosarcoma, rhabdomyosarcoma, rhabdoid cancer, and nephroblastoma (Wilm's tumor).

In some such embodiment, a compound detailed herein or a pharmaceutically acceptable salt, prodrug, metabolite, or derivative thereof, can also be used in combination with an additional therapy. The additional therapy may be optionally includes one or more therapeutic agents, radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing.

### General Reaction Schemes

The compounds of the current invention may be synthesized by varieties of methods by those skilled in the art of organic chemistry, and general synthetic schemes for preparing compounds of the present invention are described herein. These schemes are illustrative and not meant to limit the possible methodologies one skilled in the art to prepare the compounds disclosed herein. Different methods preparing the current disclosed compounds will be evident to those skilled in the art. General schemes to prepare the compounds of the present invention are given in the Examples section set out hereinafter. Preparation of homochiral examples may be realized by techniques known to one skilled in the art. For example, homochiral compounds may be prepared by separation of racemic products or diastereomers by chiral phase preparative HPLC. Alternatively, the example compounds may be prepared by methods known to give enantiomerically or diastereomerically enriched products.

The following disclosed reactions and techniques in this section are performed in solvents appropriate to the reagents and materials employed and are suitable for the transformations being effected. Also, it is to be understood that all proposed reaction conditions, including choice of solvent, reaction atmosphere, reaction temperature, duration of the experiment and work up procedures, are selected to be the conditions standard for that reaction, which should be readily recognized by one skilled in the art. It is also understood by one skilled in the art of organic synthesis that the functional groups present on various portions of the molecule must be compatible with the reagents and reactions chosen. The restrictions of the substituents that are compatible with the reaction conditions will be evident to one skilled in the art, with alternatives required should incompatible substituents are present. Sometimes a judgment to modify the order of the synthetic steps or to select one specific rout over another is required in order to obtain a desired compound of the invention. It will also be understood that the planning of any synthetic route in this field will encompass the judicious choice of a protecting group used for protection of reactive functional groups present in the compounds described in this invention (Wuts and Greene, Greene's Protective Groups in Organic Synthesis, Fourth Edition, Wiley and Sons (2007)).

The disclosed compounds of the present invention may be prepared from commercially available reagents using the synthetic methods and reaction schemes described herein, or using other reagents and conventional methods well known to those skilled in the art. For example, compounds of the present invention may be prepared using the General Reaction Schemes I or II, which may be followed by a deprotection step to deprotect a protection group to obtain the disclosed compounds.

In some embodiments, compounds of formula (II) are prepared according to the general procedure depicted in Scheme I, below.

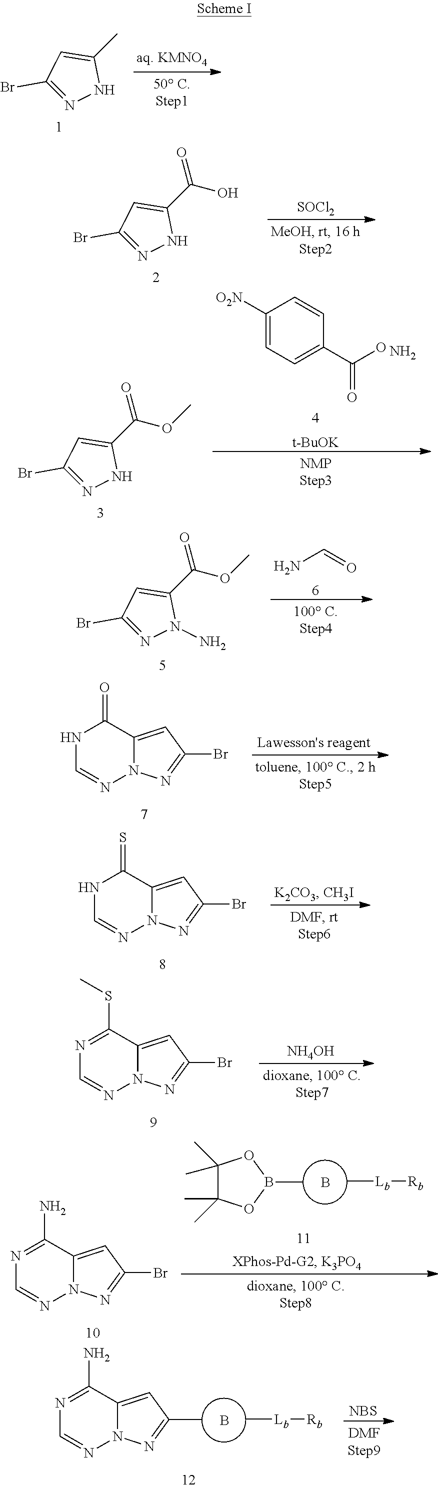

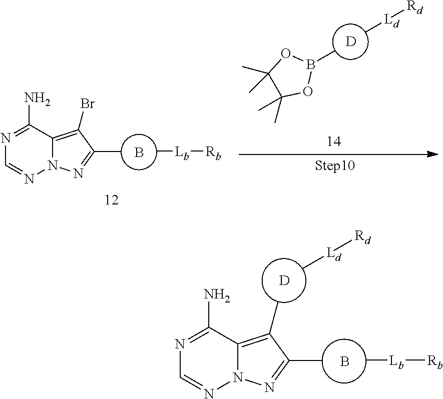

In some embodiments, Step 8 comprises the coupling of a compound of formula 10 with a synthon comprising B-L_{b}-R_{b} functionalized with a suitable reactive group, thereby forming a compound of formula 12. In some embodiments, the suitable reactive group is a boronate ester. In some embodiments, the suitable reactive group is a pinacol boronate.

In some embodiments, Step 9 comprises the bromination of a compound of formula 12. In some embodiments the reagent used is N-Bromosuccinimide.

In some embodiments, Step 10 comprises the coupling of a compound of formula 13 with a synthon comprising D-L_{d}-R_{d} functionalized with a suitable reactive group, thereby forming a compound of formula II. In some embodiments, the suitable reactive group is a boronate ester or boronic acid.

In some embodiments, compounds of formula (IV) are prepared according to the general procedure depicted in Scheme II, below.

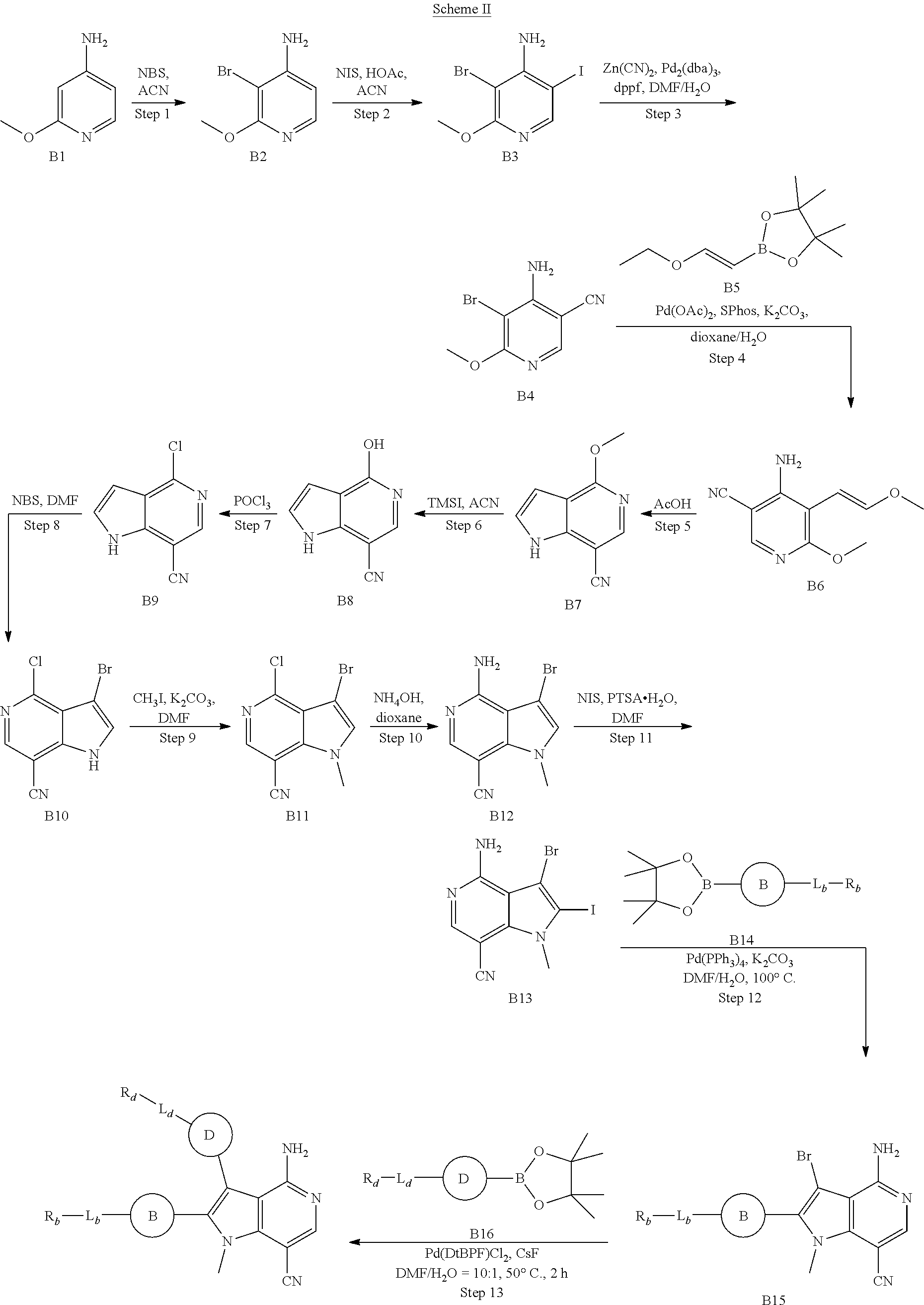

### Step 1: 3-bromo-2-methoxypyridin-4-amine

To a solution of 2-methoxypyridin-4-amine (Compound B1 which is Commercially available, 500.0 g, 4.0 mol, 1.0 eq) in ACN (5.0 L, 10.0 V) was added NBS (783.1 g, 4.4 mol, 1.1 eq) in one portion at 10° C., and the mixture was warmed to 20° C. and stirred for 1 h. The reaction mixture was poured into H₂O (7.5 L, 15.0 V) and MTBE (5.0 L, 10.0 V), then separated. The aqueous phase was extracted with MTBE (5.0 L, 10.0 V), and the combined organic phases were washed with 10 wt % NaCl aqueous (5.0 L, 10.0 V), dried over Na₂SO₄ and concentrated. The crude product was purified by silica gel chromatography eluted with Heptane/EtOAc=10/1 (2.5 L, 5.0 V) to give 3-bromo-2-methoxypyridin-4-amine (706.7 g, 93% assay, 81% yield) as a brown solid. ¹H NMR (400 MHz, DMSO-d6) δ 7.60 (d, J=5.6 Hz, 1H), 6.37 (d, J=5.6 Hz, 1H), 6.20 (s, 2H), 3.81 (s, 3H).

### Step 2: 3-bromo-5-iodo-2-methoxypyridin-4-amine

To a solution of 3-bromo-2-methoxypyridin-4-amine (706.7 g, 93% assay, 3.3 mol, 1.0 eq) in ACN (7.1 L, 10 V) was added HOAc (19.8 g, 0.33 mol, 0.1 eq) and NIS (1124.9 g, 5.0 mol, 1.5 eq) at 0° C., and the mixture was warmed to 20° C. and stirred for 2 h. H₂O (35.5 L, 50 V) was added slowly into mixture while solid was precipitated. The mixture was stirred for 30 min and filtered. The filter cake was washed with H₂O (2 V), collected and dried to give 3-bromo-5-iodo-2-methoxypyridin-4-amine (918.7 g, 94% assay, 81% yield) as a pink solid. ¹H NMR (400 MHz, DMSO-d6) δ 8.05 (s, 1H), 6.08 (s, 2H), 3.82 (s, 3H).

### Step 3: 4-amino-5-bromo-6-methoxynicotinonitrile

To a solution of 3-bromo-5-iodo-2-methoxypyridin-4-amine (410.0 g, 94% assay, 1.2 mol, 1.0 eq) in DMF (4.1 L, 10 V) was added Zn(CN)₂ (82.2 g, 0.7 mol, 0.55 eq), Pd(dba)₃ (91.6 g, 0.1 mol, 0.1 eq) and dppf (110.9 g, 0.2 mol, 0.2 eq) at 25° C. under N₂. The mixture was heated to 80° C. and stirred for 2 h. H₂O (20.5 L, 50 V) was added into mixture slowly while solid was precipitated. The mixture was filtered, and the filter cake was washed with H₂O (0.8 L, 2 V). The filter cake was dissolved with DCM (8.2 L, 20 V), washed with 10 wt % EDTA aqueous solution (4.1 L, 10 V) and 10 wt % NaCl aqueous solution (4.1 L, 10 V), dried over Na₂SO₄ and concentrated. 2 batches (410.0 g×2) were combined, and the crude product was purified by silica gel chromatography eluted with Heptane/EtOAc=5/1 to give 4-amino-5-bromo-6-methoxynicotinonitrile (342.9 g, 93% assay, 59% yield) as a yellow solid. ¹H NMR (300 MHz, DMSO-d6) δ 8.22 (s, 1H), 6.95 (s, 2H), 3.91 (s, 3H).

### Step 4: (E)-4-amino-5-(2-ethoxyvinyl)-6-methoxynicotinonitrile

To a solution of 4-amino-5-bromo-6-methoxynicotinonitrile (342.9 g, 93% assay, 1.4 mol, 1.0 eq) in 1,4-dioxane (5.1 L, 15 V) was added H₂O (0.3 L, 1 V), 4-amino-5-bromo-6-methoxynicotinonitrile (554.6 g, 2.8 mol, 2.0 eq), SPhos (41.1 g, 0.1 mol, 0.075 eq), K₂CO₃ (386.4 g, 2.8 mmol, 2.0 eq) and Pd(OAc)₂ (9.0 g, 0.04 mol, 0.03 eq) at 25° C. under N₂, and the mixture was warmed to 80° C. and stirred for 16 h. The mixture was cooled to 25° C., then EA (6.9 L, 20 V) and H₂O (6.9 L, 20 V) were added. The mixture was separated, and the aqueous layer was extracted with EA (3.5 L, 10 V). The combined organic layers were washed with 10 wt % NaCl aqueous (6.9 L, 20 V), dried with Na₂SO₄ and concentrated. The residue was purified by silica gel chromatography eluted with Heptane/EtOAc=5/1 to give (E)-4-amino-5-(2-ethoxyvinyl)-6-methoxynicotinonitrile (278.6 g, 97% assay, 89% yield) as a yellow solid. 1H NMR (400 MHz, DMSO-d6) δ 8.03 (s, 1H), 7.15 (d, J=12.7 Hz, 1H), 6.46 (s, 2H), 5.57 (d, J=12.7 Hz, 1H), 3.96-3.88 (m, 2H), 3.86 (s, 3H), 1.25 (t, J=7.0 Hz, 3H).

### Step 5: 4-methoxy-1H-pyrrolo[3,2-c]pyridine-7-carbonitrile

A solution of (E)-4-amino-5-(2-ethoxyvinyl)-6-methoxynicotinonitrile (200.0 g, 97% assay, 885.8 mmol, 1.0 eq) in AcOH (2.0 L, 10.0 V) was warmed to 100° C. and stirred for 3 h. The reaction mixture was concentrated, and MTBE (2.0 L, 10.0 V) was added into the residue. The mixture was stirred for 30 min and filtered. The filter cake was collected and dried at 50° C. to give 4-methoxy-1H-pyrrolo[3,2-c]pyridine-7-carbonitrile (151.2 g, 95% purity, 93% assay, 92% yield) as a light-pink solid. ¹H NMR (400 MHz, DMSO-d6) δ 12.46 (s, 1H), 8.31 (s, 1H), 7.47 (d, J=13.5 Hz, 1H), 6.64 (s, 1H), 4.05 (s, 3H).

### Step 6: 4-hydroxy-1H-pyrrolo[3,2-c]pyridine-7-carbonitrile

To a solution of 4-methoxy-1H-pyrrolo[3,2-c]pyridine-7-carbonitrile (150.0 g, 93% assay, 803.8 mmol, 1.0 eq) in ACN (1.5 L, 10.0 V) was added iodotrimethylsilane (160.8 g, 803.8 mmol, 1.0 eq) at 25° C., and the mixture was warmed to 80° C. and stirred for 2 h. The reaction mixture was concentrated, and heptane (1.5 L, 10.0 V) was added into the residue. The mixture was concentrated again, and MTBE/Heptane=1/1 (750.0 mL, 5.0 V) was added. The mixture was stirred for 30 min and filtered. The filter cake was collected and dried to give 4-hydroxy-1H-pyrrolo[3,2-c]pyridine-7-carbonitrile (120.5 g, 97% purity, 92% assay, 86% yield) as a yellow solid. ¹H NMR (400 MHz, DMSO-d6) δ 12.15 (s, 1H), 11.61 (s, 1H), 7.90 (d, J=20.6 Hz, 1H), 7.19-7.05 (m, 1H), 6.55 (dd, J=3.0, 2.0 Hz, 1H).

### Step 7: 4-chloro-1H-pyrrolo[3,2-c]pyridine-7-carbonitrile

To a solution of 4-hydroxy-1H-pyrrolo[3,2-c]pyridine-7-carbonitrile (100.0 g, 92% assay, 576.2 mmol, 1.0 eq) in ACN (0.3 L, 3 V) was added POCl₃ (265.0 g, 1728.6 mmol, 3.0 eq) at 25° C., and the mixture was heated to 80° C. and stirred for 4 h. The reaction mixture was cooled to 20° C. and poured into H₂O (2.0 L, 20 V) slowly. Then the mixture was adjusted to PH=7-8 with 5 wt % NaHCO₃ aqueous solution. The mixture was extracted with EA (1.5 L×2, 15 V×2), and the combined organic layers were washed with 10% NaCl aqueous (1.0 L, 10 V), dried with Na₂SO₄ and concentrated. The residue was triturated with heptane (0.5 L, 5 V) to give 4-chloro-1H-pyrrolo[3,2-c]pyridine-7-carbonitrile (105.4 g, 93% purity, 79% assay, 81% yield) as a brown solid. ¹H NMR (400 MHz, DMSO-d6) δ 12.97 (s, 1H), 8.53 (s, 1H), 7.79-7.64 (m, 1H), 6.81-6.68 (m, 1H).

### Step 8: 3-bromo-4-chloro-1H-pyrrolo[3,2-c]pyridine-7-carbonitrile

To a solution of 4-chloro-1H-pyrrolo[3,2-c]pyridine-7-carbonitrile (50.0 g, 79% assay, 221.0 mmol, 1.0 eq) in DMF (350.0 mL, 7.0 V) was added NBS (39.3 g, 221.0 mmol, 1.0 eq) at 0° C., and the mixture was stirred at 0° C. for 4 h. The mixture was poured into H₂O (1050.0 mL, 35.0 V) while solid was precipitated. The mixture was filtered, and the filter cake was triturated with MTBE (250.0 mL, 5 V) for 2 h. The mixture was filtered, and the filter cake was collected and dried to give 3-bromo-4-chloro-1H-pyrrolo[3,2-c]pyridine-7-carbonitrile (50.1 g, 98% purity, 96% assay, 85% yield) as a yellow solid. ¹H NMR (400 MHz, DMSO-d6) δ 13.36 (s, 1H), 8.58 (s, 1H), 7.97 (d, J=9.0 Hz, 1H).

### Step 9: 3-bromo-4-chloro-1-methyl-1H-pyrrolo[3,2-c]pyridine-7-carbonitrile

To a solution of 3-bromo-4-chloro-1H-pyrrolo[3,2-c]pyridine-7-carbonitrile (45.0 g, 96% assay, 169.1 mmol, 1.0 eq) in DMF (450.0 mL, 10.0 V) was added K₂CO₃ (46.7 g, 338.2 mmol, 2.0 eq) and MeI (48.0 g, 338.2 mmol, 2.0 eq) at 0° C., and the reaction mixture was heated to 25° C. and stirred for 3 h. The mixture was poured into H₂O (1.8 L, 40.0 V) while solid was precipitated. The mixture was filtered, and the filter cake was collected and dried to give 3-bromo-4-chloro-1-methyl-1H-pyrrolo[3,2-c]pyridine-7-carbonitrile (42.5 g, 98% purity, 97% assay, 90% yield) as an off-white solid. ¹H NMR (300 MHz, DMSO-d6) δ 8.56 (d, J=7.3 Hz, 1H), 7.97 (s, 1H), 4.07 (s, 3H).

### Step 10: 4-amino-3-bromo-1-methyl-1H-pyrrolo[3,2-c]pyridine-7-carbonitrile

To a solution of 3-bromo-4-chloro-1-methyl-1H-pyrrolo[3,2-c]pyridine-7-carbonitrile (40.0 g, 97% assay, 143.6 mmol, 1.0 eq) in 1,4-dioxane (200.0 mL, 5.0 V) was added aqueous solution of ammonia (200.0 mL, 5.0 V) at 25° C., and the reaction mixture was warmed to 100° C. and stirred for 16 h under closed system. The reaction mixture was concentrated, and the residue was triturated with H₂O (400.0 mL, 10 V) for 30 min. The mixture was filtered, and the filter cake was triturated with MTBE (200.0 mL, 5 V) for 2 h. The mixture was filtered, and the filter cake was collected and dried to give 4-amino-3-bromo-1-methyl-1H-pyrrolo[3,2-c]pyridine-7-carbonitrile (30.8 g, 98% purity, 97% assay, 83% yield) as a yellow solid. ¹H NMR (300 MHz, DMSO-d6) δ 8.11 (s, 1H), 7.51 (s, 1H), 6.91 (s, 2H), 3.92 (s, 3H).

### Step 11: 4-amino-3-bromo-2-iodo-1-methyl-1H-pyrrolo[3,2-c]pyridine-7-carbonitrile

To a solution of 4-amino-3-bromo-1-methyl-1H-pyrrolo[3,2-c]pyridine-7-carbonitrile (25 g, 97% assay, 96.3 mmol, 1.0 eq) in DMF (250.0 mL, 10.0 V) was added p-toluenesulfonic acid monohydrate (1.8 g, 9.6 mmol, 0.1 eq) and NIS (32.1 g, 142.7 mmol, 1.5 eq) at 0° C., and the reaction mixture was stirred at 0° C. for 3 h. The mixture was poured into H₂O (1.0 L, 40.0 V) while solid was precipitated. The mixture was filtered, and the filter cake was triturated with MTBE (250.0 mL, 10 V) for 4 h. The mixture was filtered, and the filter cake was collected and dried to give 4-amino-3-bromo-2-iodo-1-methyl-1H-pyrrolo[3,2-c]pyridine-7-carbonitrile (28.0 g, 98% purity, 92% assay, 71% yield) as a white solid. ¹H NMR (300 MHz, DMSO-d6) δ 8.08 (s, 1H), 6.99 (s, 2H), 3.97 (d, J=4.6 Hz, 3H).

### Step 12: Preparing B15

Step 12 comprises the coupling of a compound of B13 with a synthon comprising B-L_{b}-R_{b} functionalized with a suitable reactive group, thereby forming a compound of B15.

In some embodiments, the suitable reactive group is a boronate ester. In some embodiments, the suitable reactive group is a pinacol boronate.

### Step 13: Preparing Formula IV

Step 13 comprises the coupling of a compound of B15 with a synthon comprising D-L_{d}-R_{d} functionalized with a suitable reactive group, thereby forming a compound of formula IV.

In some embodiments, the suitable reactive group is a boronate ester or boronic acid.

Other compounds of Formula (I) of the present invention may be prepared by reference the Scheme I and/or Scheme II.

### EXAMPLES

The Examples provided herein describe the synthesis of compounds disclosed herein as well as intermediates used to prepare the compounds. It is to be understood that individual steps described herein may be combined. It is also to be understood that separate batches of a compound may be combined and then carried forth in the next synthetic step.

In the following description of the Examples, specific embodiments are described. These embodiments are described in sufficient detail to enable those skilled in the art to practice certain embodiments of the present disclosure. Other embodiments may be utilized and logical and other changes may be made without departing from the scope of the disclosure. The following description is, therefore, not intended to limit the scope of the present disclosure, but rather is specified by the claims appended hereto.

TABLE 1
shows the part abbreviations of the present invention

| AQ. | Aqueous |
|---|---|
| aq. | aqueous |
| ACN | Acetonitrile |
| B₂Pin₂ | Bis(pinacolato)diboron |
| Bn | benzyl |
| BINAP | (±)-2,2′-Bis(diphenylphosphino)-1,1′-binaphthalene |
| Boc | tert-butoxycarbonyl |
| Boc₂O | di-tert-butyl dicarbonate |
| ° C. | Degrees centigrade |
| DCM | dichloromethane |
| DIAD | diisopropyl azodicarboxylate |
| DIBAL | diisobutyl aluminium hydride |
| DIEA | diisopropylethylamine |
| DMAP | 4-dimethylaminopyridine |
| DMF | dimethylformamide |
| DMSO | dimethyl sulfoxide |
| EA | ethyl acetate |
| equiv. | equivalent |
| Et | ethyl |
| FCC | flash column chromatography |
| g | gram |
| HATU | 1-[Bis(dimethylamino)methylene]-1H-1,2,3- |
| | triazolo[4,5-b]pyridinium 3-oxid hexafluorophosphate |
| HPLC | high pressure liquid chromatography |
| LC-MS or | Liquid Chromatography-Mass Spectroscopy |
| LC/MS | |
| Me | Methyl |
| min | minute(s) |
| mL | milliliter(s) |
| mmol | millimolar |
| MTBE | methyl t-butyl ether |
| NBS | N-Bromosuccinimide |
| NIS | N-Iodosuccinimide |
| NMR | nuclear magnetic resonance |
| NMP | N-Methyl-2-pyrrolidone |
| Pd₂(dba)₃ | Tris (dibenzylideneacetone)dipalladium |
| Pd(dppf)Cl₂ | [1,1′-bis(diphenylphosphino) |
| | ferrocene]dichloropalladium(II) |
| Pd(DtBPF)Cl₂ | 1,1′-Bis (di-t-butylphosphino)ferrocene palladium |
| | dichloride (II) |
| Pd(OAc)₂ | polladium (II) acetate |
| Pd(PPh₃)₄ | Palladium(0) tetrakis(triphenylphosphine) |
| PE | polyether |
| PTSA | p-toluenesulfonic acid |
| Prep-TLC | prep thin layer chromatography |
| RuPhos | Dicyclohexyl(2′,6′-diisopropoxy-[1,1′-biphenyl]-2- |
| | yl)phosphine |
| sat. | saturated |
| T₃P | Propylphosphonic anhydride |
| TEA | triethylamine |
| TFA | Trifluoroacetic acid |
| TMSI | iodotrimethylsilane |
| THF | Tetrahydrofuran |
| TLC | Thin layer chromatography |
| rt or r.t. | room temperature |
| XPhos-Pd-G2 | Chloro(2-dicyclohexylphosphino-2′,4′,6′-triisopropyl- |
| | 1,1′-biphenyl)[2-(2′-amino-1,1′-biphenyl)]palladium(II) |

### Example 1

### N-(4-(4-amino-5-(3-methoxy-4-((4-methylpyrimidin-2-yl)oxy)phenyl)pyrazolo[5,1-f][1,2,4]triazin-6-yl)phenyl)acrylamide (“Compound 1”)

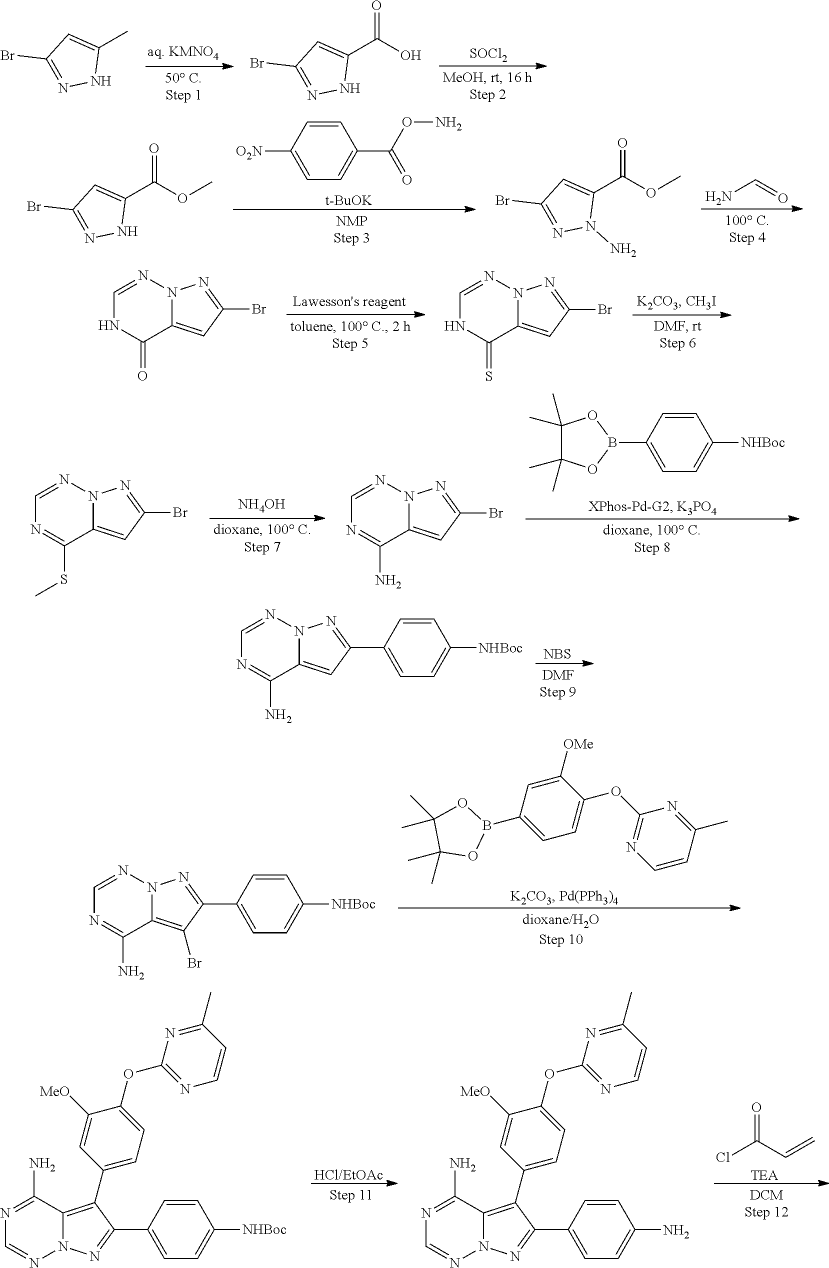

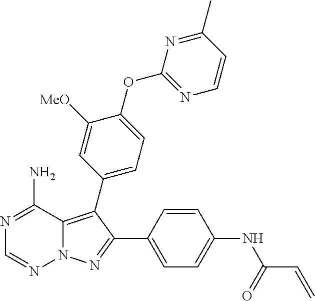 [SMILES: C=CC(=O)Nc1ccc(-c2nn3ncnc(N)c3c2-c2ccc(Oc3nccc(C)n3)c(OC)c2)cc1]

### Step 1: 3-bromo-1H-pyrazole-5-carboxylic acid

A round bottomed flask was charged with 3-bromo-5-methyl-1H-pyrazole (2.5 g, 15.5 mmol), KMnO₄ (4.9 g, 30.1 mmol), H₂O (100 mL) and a stir bar. The reaction mixture was stirred at 95° C. for 6 h. The reaction mixture was cooled to r.t., then filtered. The filtrate was collected and extracted with EA (50 mL) three times. The combined organic layers were washed with brines, dried over sodium sulfate, filtered, and concentrated in vacuo. The resulting crude material was purified by silica gel chromatography. Concentration in vacuo resulted in 3-bromo-1H-pyrazole-5-carboxylic acid (2.30 g, 78%) as white solid. MS (ESI): mass calcd. for C₄H₃BrN₂O₂: 190; m/z found 191 [M+1]⁺. ¹H NMR (400 MHz, DMSO) δ 14.15 (s, 1H), 13.59 (s, 1H), 6.87 (s, 1H).

### Step 2: Methyl 3-bromo-1H-pyrazole-5-carboxylate

A round bottomed flask was charged with 3-bromo-1H-pyrazole-5-carboxylic acid (2.3 g, 12 mmol), MeOH (20 mL) and a stir bar. The reaction mixture was cooled to 0° C. and SOCl₂ (1.56 g, 13.2 mmol) was dropwise added. Then the reaction mixture was stirred at 60° C. for 2 h. The reaction mixture was cooled to r.t. and was poured into ice water. The aqueous phase was extracted with EA (20 mL) three times. The combined organic layers were washed with brines, dried over sodium sulfate, filtered, and concentrated in vacuo. The resulting crude material was purified by silica gel chromatography. Concentration in vacuo resulted in Methyl 3-bromo-1H-pyrazole-5-carboxylate (2.20 g, 93%) as white solid. MS (ESI): mass calcd. for C₅H₅BrN₂O₂: 204; m/z found 205 [M+1]⁺. ¹H NMR (400 MHz, DMSO) δ 14.36 (s, 1H), 6.97 (s, 1H), 3.85 (s, 3H).

### Step 3: Methyl 1-amino-3-bromo-1H-pyrazole-5-carboxylate

A round bottomed flask was charged with Methyl 3-bromo-1H-pyrazole-5-carboxylate (2.2 g, 10.7 mmol), NMP (10 ml) and a stir bar. A solution of t-BuOK (1-32 g, 11.8 mmol) in NMP (5 mL) was added, then the mixture was stirred at 20° C. for 20 min. A solution of O-(4-nitrobenzoyl)hydroxylamine (2.15 g, 11.8 mmol) was added, and the reaction mixture was stirred at below 25° C. for 2 h. The reaction mixture was diluted with 10% aqueous NaCl (25 mL) and EA (25 mL). The aqueous phase was extracted with EA (25 mL) three times. The combined organic layers were washed with 5% NaHCO₃ (15 mL), H₂O (15 mL) three times, dried over sodium sulfate, filtered, and concentrated in vacuo. The resulting crude material was purified by silica gel chromatography (EA:PE=17:83). Concentration in vacuo resulted in Methyl 1-amino-3-bromo-1H-pyrazole-5-carboxylate (2.00 g, 84%) as white solid. MS (ESI): mass calcd. for C₅H₆BrN₃O₂: 219; m/z found 220 [M+1]⁺. ¹H NMR (400 MHz, DMSO) δ 6.96 (s, 2H), 6.89 (s, 1H), 3.83 (s, 3H).

### Step 4: 6-bromopyrazolo[5,1-f][1,2,4]triazin-4(3H)-one

A round bottomed flask was charged with Methyl 1-amino-3-bromo-1H-pyrazole-5-carboxylate (2.00 g, 9.1 mmol), methanamide (5.0 mL) and a stir bar. The reaction mixture was irradiated in the microwave at 150° C. for 2 h. The reaction mixture was cooled to r.t. and poured into water. The aqueous phase was extracted with EA (20 mL) three times. The combined organic layers were washed with brines, dried over sodium sulfate, filtered, and concentrated in vacuo. The resulting crude material was purified by silica gel chromatography. Concentration in vacuo resulted in 6-bromopyrazolo[5,1-f][1,2,4]triazin-4(3H)-one (1.00 g, 51%) as white solid. MS (ESI): mass calcd. for C₅H₃BrN₄O: 214; m/z found 215 [M+1]⁺. ¹H NMR (400 MHz, DMSO) δ 12.58 (s, 1H), 8.12 (s, 1H), 7.24 (s, 1H).

### Step 5: 6-bromopyrazolo[5,1-f][1,2,4]triazine-4(3H)-thione

A round bottomed flask was charged with 6-bromopyrazolo[5,1-f][1,2,4]triazin-4(3H)-one (100 mg, 0.46 mmol), Lawesson's reagent (93 mg, 0.23 mmol), toluene (5 mL) and a stir bar. The reaction mixture was stirred at 100° C. for 2 h. Then the reaction mixture was cooled to r.t. The reaction mixture was concentrated in vacuo to obtain the crude product. The crude product was purified by pre-TLC (PE:EA=1:1) to afford 6-bromopyrazolo[5,1-f][1,2,4]triazine-4(3H)-thione (80 mg, 74%) as yellow solid. MS (ESI): mass calcd. for C₅H₃BrN₄S: 230; m/z found 231 [M+1]⁺.

### Step 6: 6-bromo-4-(methylthio)pyrazolo[5,1-f][1,2,4]triazine

A round bottomed flask was charged with 6-bromopyrazolo[5,1-f][1,2,4]triazine-4(3H)-thione (80 mg, 0.38 mmol), K₂CO₃ (53 mg, 0.38 mmol), CH₃I (60 mg, 0.42 mmol) and DMF (3 mL). The reaction mixture was stirred at r.t. for 2 h. The reaction mixture was poured to water (15 mL). The aqueous was extracted with DCM (10 mL) three times. The combined organic layers were concentrated in vacuo to afford 6-bromo-4-(methylthio)pyrazolo[5,1-f][1,2,4]triazine (60 mg, 71%) as white solid. MS (ESI): mass calcd. for C₆H₅BrN₄S: 244; m/z found 245 [M+1]⁺. ¹H NMR (400 MHz, DMSO) δ 8.69 (s, 1H), 7.37 (s, 1H), 2.72 (s, 1H).

### Step 7: 6-bromopyrazolo[5,1-f][1,2,4]triazin-4-mine

A round bottomed flask was charged with 6-bromo-4-(methylthio)pyrazolo[5,1-f][1,2,4]triazine (60 mg, 0.24 mmol), NH₃—CH₃OH (3 mL). The reaction mixture was stirred under 50 psi at 100° C. for 12 h in a 50 mL of autoclave. The solvent was evaporated in vacuo to afford crude 6-bromopyrazolo[5,1-f][1,2,4]triazin-4-amine (50 mg) as yellow solid. MS (ESI): mass calcd. For C₅H₄BrN₅: 213; m/z found 214 [M+1]⁺.

### Step 8: tert-butyl (4-(4-aminopyrazolo[5,1-f][1,2,4]triazin-6-yl)phenyl)carbamate

A round bottomed flask was charged with 6-bromopyrazolo[5,1-f][1,2,4]triazin-4-amine (30 mg, 0.14 mmol), XPhos-Pd-G2 (11 mg, 0.014 mmol), K₃PO₄ (60 mg, 0.28 mmol), tert-butyl (4-(4,4,5,5-tetramethyl-1,3,2-dioxaborolan-2-yl)phenyl)carbamate (38 mg, 0.15 mmol), dioxane (5 mL), H₂O (1 mL) and a stir bar. The reaction mixture was stirred under N₂ at 100° C. for 3 h. The reaction mixture was cooled to r.t. The reaction mixture was extracted with EtOAc (15 mL) three times. The combined organic layers were washed with brines, dried over sodium sulfate, filtered, and concentrated in vacuo.

The resulting crude material was purified by pre-TLC (PE:EA=3:1). Concentration in vacuo resulted in tert-butyl (4-(4-aminopyrazolo[5,1-f][1,2,4]triazin-6-yl)phenyl)carbamate (20 mg, 50%) as yellow solid. MS (ESI): mass calcd. for C₁₆H₁₈N₆O₂: 326; m/z found 327 [M+1]⁺.

### Step 9: tert-butyl (4-(4-amino-5-bromopyrazolo[5,1-f][1,2,4]triazin-6-yl)phenyl)carbamate

A round bottomed flask was charged with tert-butyl (4-(4-aminopyrazolo[5,1-f][1,2,4]triazin-6-yl)phenyl)carbamate (10 mg, 0.03 mmol), NBS (5 mg, 0.03 mmol), ACN (3 mL) and a stir bar. The reaction mixture was stirred at r.t. for 10 min. The reaction mixture was poured to water and filtrated. The filter cake was collected to afford tert-butyl (4-(4-amino-5-bromopyrazolo[5,1-f][1,2,4]triazin-6-yl)phenyl)carbamate (10 mg, 80%) as white solid. MS (ESI): mass calcd. for C₁₆H₁₇BrN₆O₂: 404; m/z found 405 [M+1]⁺.

### Step 10: tert-butyl(4-(4-amino-5-(3-methoxy-4-((4-methylpyrimidin-2-yl)oxy)phenyl)pyrazolo[5,1-f][1,2,4]triazin-6-yl)phenyl)carbamate

A round bottomed flask was charged with tert-butyl (4-(4-amino-5-bromopyrazolo[5,1-f][1,2,4]triazin-6-yl)phenyl)carbamate (20 mg, 0.05 mmol), 2-(2-methoxy-4-(4,4,5,5-tetramethyl-1,3,2-dioxaborolan-2-yl)phenoxy)-4-methylpyrimidine (19 mg, 0.055 mmol), Pd(PPh₃)₄ (0.6 mg, 0.005 mmol), K₂CO₃ (14 mg, 0.1 mmol), dioxane (5 mL), H₂O (1 mL) and a stir bar under N₂. The reaction mixture was stirred at 100° C. under N₂ for 3 h. The reaction mixture was cooled to r.t. and poured into water (15 mL). The aqueous layer was extracted with EA (5 mL) three times. The combined organic layers were washed with brines, dried over sodium sulfate, filtered, and concentrated in vacuo. The crude was purified by pre-TLC (PE:EA=1:1) to afford tert-butyl(4-(4-amino-5-(3-methoxy-4-((4-methylpyrimidin-2-yl)oxy)phenyl)pyrazolo[5,1-f][1,2,4]triazin-6-yl)phenyl)carbamate (22 mg, 82%) as white solid. MS (ESI): mass calcd. for C₂₈H₂₈N₈O₄ 540; m/z found 541 [M+1]⁺.

### Step 11: 6-(4-aminophenyl)-5-(3-methoxy-4-((4-methylpyrimidin-2-yl)oxy)phenyl) pyrazolo[5,1-f][1,2,4]triazin-4-amine

A round bottomed flask was charged with tert-butyl(4-(4-amino-5-(3-methoxy-4-((4-methylpyrimidin-2-yl)oxy)phenyl)pyrazolo[5,1-f][1,2,4]triazin-6-yl)phenyl)carbamate (22 mg, 0.04 mmol) in HCl/EtOAc. The reaction mixture was stirred at r.t. for 1 h, then concentrated to afford 6-(4-aminophenyl)-5-(3-methoxy-4-((4-methylpyrimidin-2-yl)oxy)phenyl)pyrazolo[5,1-f][1,2,4]triazin-4-amine (18 mg, 100%) as white solid. MS (ESI): mass calcd. for C₂₃H₂ON₈O₂: 440; m/z found 441 [M+1]⁺.

### Step 12: N-(4-(4-amino-5-(3-methoxy-4-((4-methylpyrimidin-2-yl)oxy)phenyl)pyrazolo[5,1-f][1,2,4]triazin-6-yl)phenyl)acrylamide

A round bottomed flask was charged with 6-(4-aminophenyl)-5-(3-methoxy-4-((4-methylpyrimidin-2-yl)oxy)phenyl)pyrazolo[5,1-f][1,2,4]triazin-4-amine, DCM and a stir bar. The reaction mixture was cooled to t 0° C., then TEA (1 drop) and acryloyl chloride (3.6 mg, 0.04 mmol) were added under N₂. The reaction mixture was stirred at 0° C. for 10 min, then poured to ice water. The aqueous layer was extracted with DCM (5 mL) three times. The combined organic layers were washed with brines, dried over sodium sulfate, filtered, and concentrated in vacuo. The crude product was purified by pre-HPLC to afford Compound 1 (1.2 mg, 6%) as white solid. MS (ESI): mass calcd. For C₂₆H₂₂N₈O₃: 494; m/z found 495 [M+H]⁺. ¹H NMR (400 MHz, MeOD) δ 8.40 (d, J=5.1 Hz, 1H), 8.08 (s, 1H), 7.62 (d, J=8.7 Hz, 2H), 7.51 (d, J=8.8 Hz, 2H), 7.32 (d, J=8.0 Hz, 1H), 7.17 (d, J=1.9 Hz, 1H), 7.14 (d, J=5.1 Hz, 1H), 7.09 (dd, J=8.0, 1.9 Hz, 1H), 6.39 (dd, J=13.7, 5.9 Hz, 2H), 5.77 (dd, J=9.6, 2.3 Hz, 1H), 3.66 (s, 3H), 2.52 (s, 3H).

### Example 28

### 4-(2-(4-acrylamidophenyl)-4-amino-7-cyano-1-methyl-1H-pyrrolo[3,2-c]pyridin-3-yl)-2-methoxy-N-(2,2,2-trifluoroethyl)benzamide(“Compound 28”)

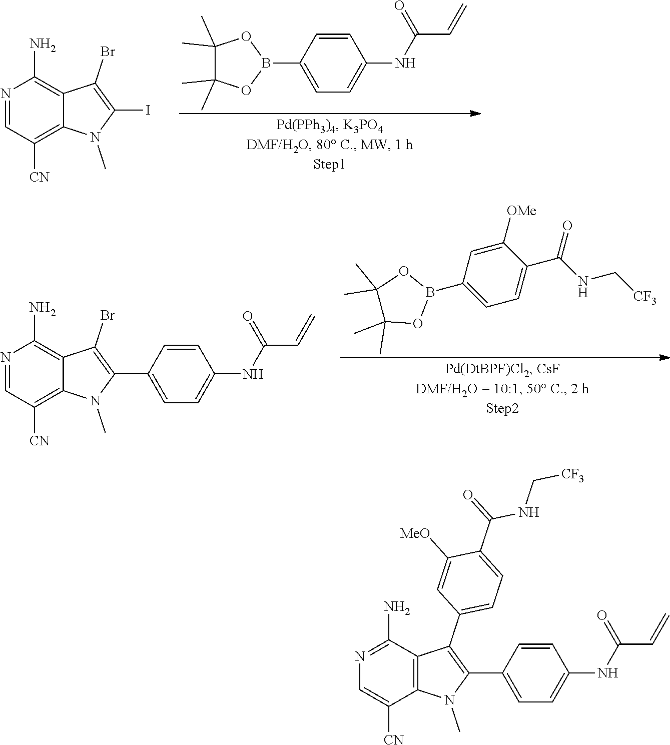

### Step 1: N-(4-(4-amino-3-bromo-7-cyano-1-methyl-1H-pyrrolo[3,2-c]pyridin-2-yl)phenyl)acrylamide

To a solution of 4-amino-3-bromo-2-iodo-1-methyl-1H-pyrrolo[3,2-c]pyridine-7-carbonitrile (300 mg, 0.80 mmol), N-(4-(4,4,5,5-tetramethyl-1,3,2-dioxaborolan-2-yl)phenyl) acrylamide (240 mg, 0.88 mmol) and K₃PO₄ (504 mg, 2.4 mmol) in dioxane (10 ml) and H₂O (1.0 ml), Pd(PPh₃)₄ (92 mg, 0.08 mmol) was added and the reaction system was purged with nitrogen three times. The reaction mixture was irradiated in the microwave on a Biotage Smith Synthesizer at 80° C. for 1 hour. The reaction mixture was concentrated in vacuo. The residue was washed with EtOAc (20 mL) and H₂O (20 mL) to obtain the desired product (226 mg) as a yellow solid. MS (ESI): mass cacld. For C₁₈H₁₄BrN₅O: 395; m/z found 396 [M+1]⁺.

### Step 2: 4-(2-(4-acrylamidophenyl)-4-amino-7-cyano-1-methyl-1H-pyrrolo[3,2-c]pyridin-3-yl)-2-methoxy-N-(2,2,2-trifluoroethyl)benzamide

To a solution of N-(4-(4-amino-3-bromo-7-cyano-1-methyl-1H-pyrrolo[3,2-c]pyridin-2-yl)phenyl) acrylamide (100 mg, 0.25 mmol), 2-methoxy-4-(4,4,5,5-tetramethyl-1,3,2-dioxaborolan-2-yl)-N-(2,2,2-trifluoroethyl)benzamide (136 mg, 0.38 mmol), CsF (114 mg, 0.75 mmol) in DMF (1 ml) and H₂O (0.1 ml), Pd(DtBPF)Cl₂ (33 mg, 0.05 mmol) was added and the reaction system was purged with nitrogen three times. The mixture was stirred for 10 min at 50° C. The mixture was quenched with water (10 mL) and extracted with EtOAc (10 mL×3). The combined organic layers were dried over Na₂SO₄ and concentrated under reduced pressure to give a residue. The residue was purified prep-HPLC (NH₃) to obtain Compound 28 (5.78 mg) as a white solid. MS (ESI): mass calcd. For C₂₈H₂₃F₃N₆O₃: 548; m/z found 549 [M+H]⁺. ¹H NMR (400 MHz, DMSO) δ 10.28 (s, 1H), 8.64 (t, J=6.4 Hz, 1H), 8.21 (s, 1H), 7.72-7.64 (m, 3H), 7.30 (d, J=8.4 Hz, 2H), 6.97 (s, 1H), 6.89 (d, J=8.4 Hz, 1H), 6.43 (m, 1H), 6.30-6.24 (m, 1H), 6.09 (s, 1H), 5.81-5.75 (m, 1H), 4.15-4.01 (m, 2H), 3.81 (s, 3H), 3.73 (s, 3H).

### Example 71

### 4-(4-amino-6-(4-(2-fluoroacrylamido)phenyl) pyrazolo[5,1-f][1,2,4]triazin-5-yl)-2-methoxy-N-(2,2,2-trifluoroethyl)benzamide (“Compound 71”)

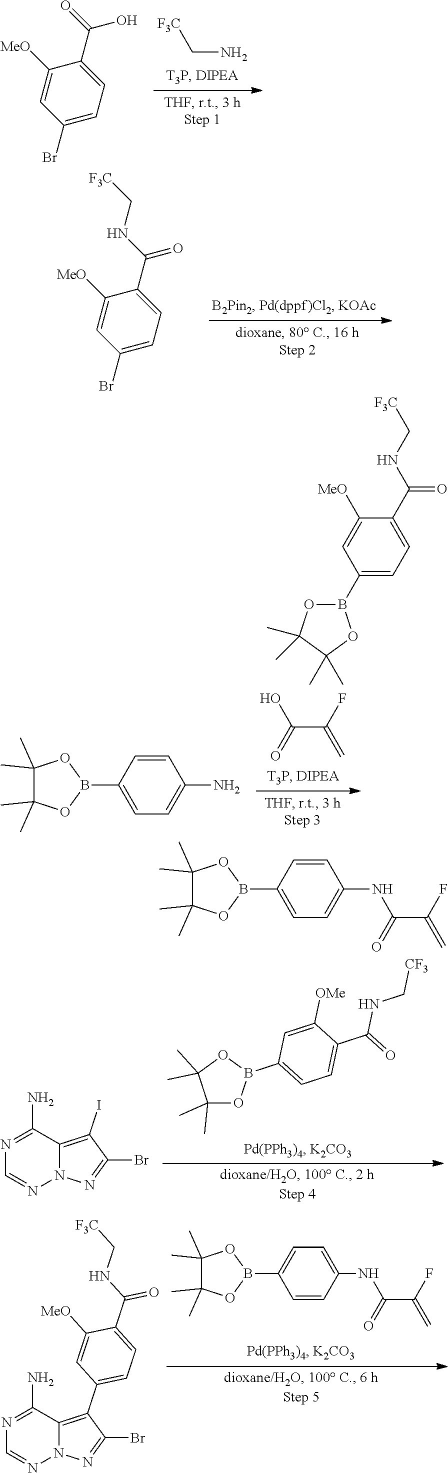

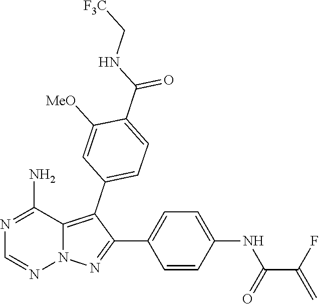 [SMILES: C=C(F)C(=O)Nc1ccc(-c2nn3ncnc(N)c3c2-c2ccc(C(=O)NCC(F)(F)F)c(OC)c2)cc1]

### Step 1: 4-bromo-2-methoxy-N-(2,2,2-trifluoroethyl)benzamide

The mixture of 4-bromo-2-methoxybenzoic acid (800 mg, 3.5 mmol), 2,2,2-trifluoroethan-1-amine (412 mg, 4.2 mmol), T3P (6.6 g, 10.4 mmol) and DIPEA (1.8 g, 13.8 mmol) in THF (10 mL) was stirred at r.t. for 3 h. The reaction mixture was diluted with H₂O (10 ml) and the solution was extracted with EtOAc (30 ml×3), dried over Na₂SO₄, filtered and concentrated under reduced pressure. The residue was purified by FCC (PE/EA=4/1) to give the product (800 mg, 74% yield) as a white solid. MS (ESI): mass cacld. for C₁₀H₉BrF₃NO₂: 311; m/z found 312 [M+H]⁺.

### Step 2: 2-methoxy-4-(4,4,5,5-tetramethyl-1,3,2-dioxaborolan-2-yl)-N-(2,2,2-trifluoroethyl)benzamide

A solution of 4-bromo-2-methoxy-N-(2,2,2-trifluoroethyl)benzamide (800 mg, 2.6 mmol), B₂Pin₂ (0.98 g, 3.8 mmol), Pd(dppf)Cl₂ (187 mg, 0.26 mmol) and KOAc (754 mg, 7.7 mmol) in dioxane (10 mL) was stirred at 80° C. for 16 h under N₂. The reaction mixture was concentrated under reduced pressure. The residue was purified by column chromatography (PE/EtOAc=4/1) to give the product (900 mg, 98% yield) as a brown solid. MS (ESI): mass calcd. for C₁₆H₂₁BF₃NO₄ 359; m/z found 360 [M+H]⁺.

### Step 3: 2-fluoro-N-(4-(4,4,5,5-tetramethyl-1,3,2-dioxaborolan-2-yl)phenyl) acrylamide

The solution of 4-(4,4,5,5-tetramethyl-1,3,2-dioxaborolan-2-yl) aniline (1.00 g, 4.6 mmol), 2-fluoroacrylic acid (493 mg, 5.5 mmol), T3P (8.7 g, 13.7 mmol) and DIPEA (2.36 g, 18.2 mmol) in THF (20 mL) was stirred at r.t. for 3 h. The residue was extracted with EtOAc and the organic layer was collected, then concentrated in vacuo. The crude was purified by FCC (PE/EA=10/1) to give the product (1.27 g, 96% yield) as a white solid. MS (ESI): mass cacld. for C₁₅H₁₉BFNO₃: 291; m/z found 292 [M+H]⁺.

### Step 4: 4-(4-amino-6-bromopyrazolo[5,1-f][1,2,4]triazin-5-yl)-2-methoxy-N-(2,2,2-trifluoroethyl)benzamide

A solution of 2-methoxy-4-(4,4,5,5-tetramethyl-1,3,2-dioxaborolan-2-yl)-N-(2,2,2-trifluoroethyl)benzamide (148 mg, 0.41 mmol), 6-bromo-5-iodopyrazolo[5,1-f][1,2,4]triazin-4-amine (200 mg, 0.59 mmol), Pd(PPh₃)₄ (68 mg, 0.04 mmol) and K₂CO₃ (245 mg, 1.77 mmol) in dioxane (2 mL) and H₂O (0.2 mL) was stirred at 100° C. for 2 h. The reaction mixture was diluted with H₂O (15 ml) and the solution was extracted with EtOAc (20 ml×3). The combined organic layers were washed with brine (10 ml), dried over Na₂SO₄, filtered and concentrated to give the residue. The residue was purified by FCC (PE/EtOAc=1/1) to give the product (100 mg, 55% yield) as a yellow oil. MS (ESI): mass calcd. for C₁₅H₁₂BrF₃N₆O₂ 444; m/z found 445 [M+H]⁺.

### Step 5: 4-(4-amino-6-(4-(2-fluoroacrylamido)phenyl) pyrazolo[5,1-f][1,2,4]triazin-5-yl)-2-methoxy-N-(2,2,2-trifluoroethyl)benzamide

A solution of 4-(4-amino-6-bromopyrazolo[5,1-f][1,2,4]triazin-5-yl)-2-methoxy-N-(2,2,2-trifluoroethyl)benzamide (100 mg, 0.22 mmol), 2-fluoro-N-(4-(4,4,5,5-tetramethyl-1,3,2-dioxaborolan-2-yl)phenyl) acrylamide (98 mg, 0.34 mmol), Pd(PPh₃)₄ (25 mg, 0.02 mmol) and K₂CO₃ (91 mg, 0.66 mmol) in dioxane (5 mL) and H₂O (0.5 mL) was stirred at 100° C. for 16 h. The reaction mixture was concentrated and the residue was purified by Prep-HPLC to give the Compound 71 (22.2 mg, 19% yield) as a white solid. MS (ESI): mass calcd. for C₂₄H₁₉F₄N₇O₃: 529; m/z found 530 [M+H]⁺. ¹H NMR (400 MHz, DMSO) δ 10.36 (s, 1H), 8.73 (t, J=6.4 Hz, 1H), 8.36 (s, 1H), 8.17 (s, 1H), 7.84 (d, J=7.8 Hz, 1H), 7.68 (d, J=8.8 Hz, 2H), 7.41 (d, J=8.8 Hz, 2H), 7.25 (s, 1H), 7.04 (dd, J=7.9, 1.1 Hz, 1H), 5.82-5.39 (m, 3H), 4.21-4.07 (m, 2H), 3.84 (s, 3H).

### Example 96

### 4-(4-amino-7-cyano-2-(4-(2-fluoroacrylamido)phenyl)-1-methyl-1H-pyrrolo[3,2-c]pyridine-3-yl)-2-methoxy-N-(2,2,2-trifluoroethyl)benzamide (“Compound 96”)

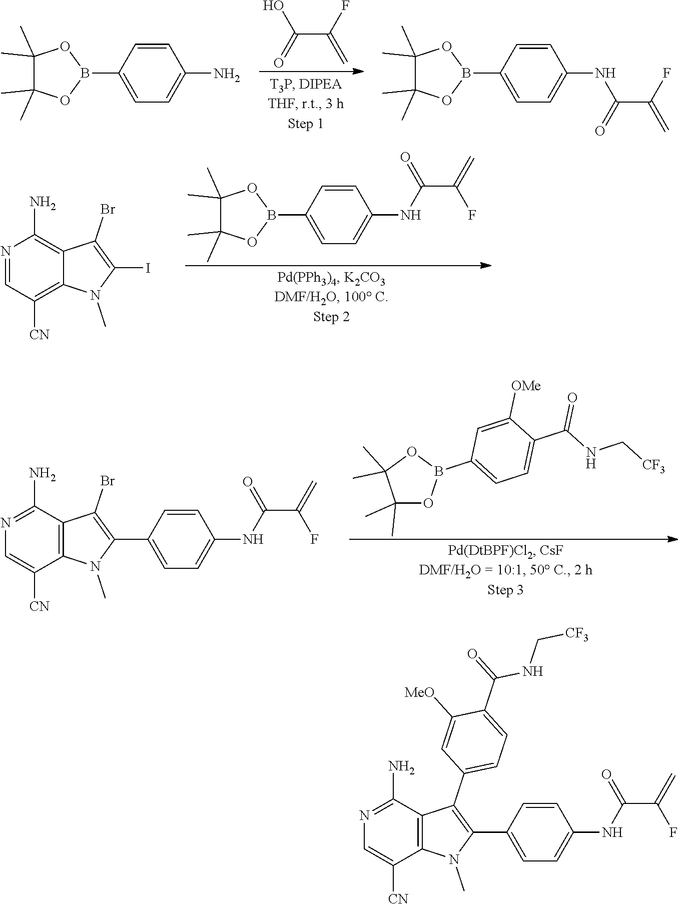

### Step 1: 2-fluoro-N-(4-(4,4,5,5-tetramethyl-1,3,2-dioxaborolan-2-yl)phenyl)acrylamide

The mixture of 4-(4,4,5,5-tetramethyl-1,3,2-dioxaborolan-2-yl) aniline (1.00 g, 4.56 mmol), 2-fluoroacrylic acid (493 mg, 5.5 mmol), T3P (8.70 g, 13.7 mmol), DIPEA (2.36 g, 18.2 mmol) in THE (20 mL) was stirred at rt for 3 h. The reaction mixture was diluted with H₂O (10 ml), extracted by EtOAc. The organic layer was collected, then dried over Na₂SO₄, filtered and concentrated in vacuo. The crude was purified by FCC (PE/EA=10/1) to give the product (1.27 g, 96% yield) as a white solid. MS (ESI): mass cacld. for C₁₅H₁₉BFNO₃ 291, m/z found 292 [M+H]⁺.

### Step 2: N-(4-(4-amino-3-bromo-7-cyano-1-methyl-1H-pyrrolo[3,2-c]pyridin-2-yl)phenyl)-2-fluoroacrylamide

To a solution of 4-amino-3-bromo-2-iodo-1-methyl-1H-pyrrolo[3,2-c]pyridine-7-carbonitrile (200 mg, 0.53 mmol) and 2-fluoro-N-(4-(4,4,5,5-tetramethyl-1,3,2-dioxaborolan-2-yl)phenyl) acrylamide (154 mg, 0.53 mmol) in DMF/H₂O (5 mL/1 mL), K₂CO₃ (220 mg, 1.59 mmol), Pd(PPh₃)₄ (58 mg, 0.05 mmol) were added. The reaction mixture was stirred at 100° C. for 2 h under N₂ protection. The mixture was concentrated in vacuo and the residue was purified by gel-column chromatography (DCM: MeOH=10:1) to give N-(4-(4-amino-3-bromo-7-cyano-1-methyl-1H-pyrrolo[3,2-c]pyridin-2-yl)phenyl)-2-fluoroacrylamide (200 mg, 91% yield) as a white solid. MS (ESI): mass calcd. for C₁₈H₁₃BrFN₅O: 413; m/z found 414 [M+H]⁺.

### Step 3: 4-(4-amino-7-cyano-2-(4-(2-fluoroacrylamido)phenyl)-1-methyl-11H-pyrrolo[3,2-c]pyridine-3-yl)-2-methoxy-N-(2,2,2-trifluoroethyl)benzamide

To a solution of N-(4-(4-amino-3-bromo-7-cyano-1-methyl-1H-pyrrolo[3,2-c]pyridin-2-yl)phenyl)-2-fluoroacrylamide (100 mg, 0.24 mmol) and 2-methoxy-4-(4,4,5,5-tetramethyl-1,3,2-dioxaborolan-2-yl)-N-(2,2,2-trifluoroethyl)benzamide (129 mg, 0.36 mmol) in DMF/H₂O (5 mL/1 mL) were added CsF (109 mg, 0.72 mmol) and Pd(DtBPF)Cl₂ (28 mg, 0.024 mmol). The reaction mixture was stirred at 50° C. for 2 h under N₂ protection. The mixture was concentrated in vacuo and the residue was purified by Prep-HPLC to give Compound 96 (31.42 mg) as white solid. MS (ESI): mass calcd. for C₂₈H₂₂F₄N₆O₃: 566; m/z found 567 [M+H]⁺. ¹H NMR (400 MHz, DMSO) δ 10.41 (s, 1H), 8.65 (t, J=6.4 Hz, 1H), 8.22 (s, 1H), 7.76 (d, J=8.8 Hz, 2H), 7.69 (d, J=7.6 Hz, 1H), 7.33 (d, J=8.4 Hz, 2H), 6.98 (d, J=0.8 Hz, 1H), 6.89 (m, 1H), 6.10 (s, 2H), 5.72 (m, 1H), 5.45 (m, 1H), 4.09 (m, 2H), 3.81 (s, 3H), 3.74 (s, 3H).

### Example 100

### 4-(4-amino-6-(2-fluoro-4-(2-fluoroacrylamido)phenyl)pyrazolo[5,1-f][1,2,4]triazin-5-yl)-2-methoxy-N-(2,2,2-trifluoroethyl)benzamide (“Compound 100”)

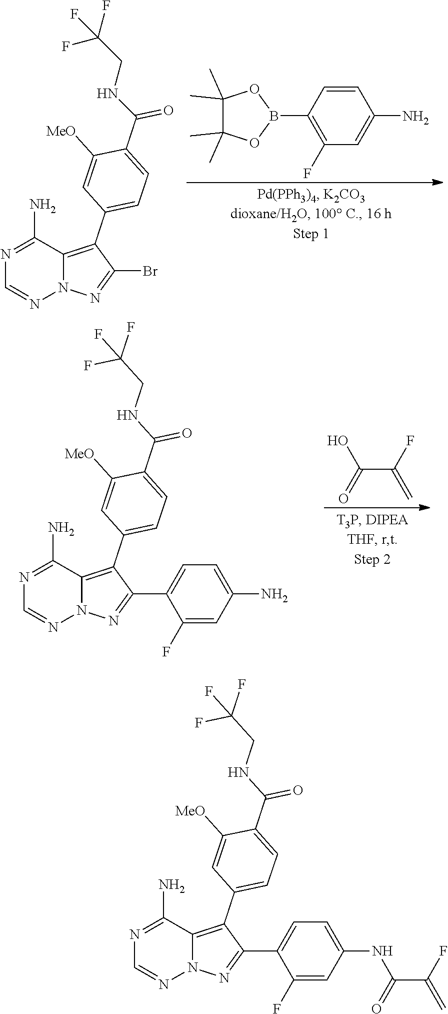

### Step 1: 4-(4-amino-6-(4-amino-2-fluorophenyl)pyrazolo[5,1-f][1,2,4]triazin-5-yl)-2-methoxy-N-(2,2,2-trifluoroethyl)benzamide

The solution of 4-(4-amino-6-bromopyrazolo[5,1-f][1,2,4]triazin-5-yl)-2-methoxy-N-(2,2,2-trifluoroethyl)benzamide (100 mg, 0.22 mmol), 3-fluoro-4-(4,4,5,5-tetramethyl-1,3,2-dioxaborolan-2-yl)aniline (80 mg, 0.34 mmol), K₂CO₃ (61 mg, 0.44 mmol) and Pd(PPh₃)₄ (23 mg, 0.02 mmol) in Dioxane (2.5 mL) and H₂O (0.7 mL) was stirred at 100° C. for 16 h under N₂ atmosphere. The mixture was quenched with H₂O and extracted with EtOAc. The combined organic phases were washed with brine, dried over Na₂SO₄, concentrated in vacuo, and purified by silica gel chromatography column (DCM/MeOH=95:5 to 10:1), to give the product (60 mg, 60% yield) as a yellow solid. LCMS (ESI) calcd. For C₂₁H₁₇F₄N₇O₂: 476; m/z found 476 [M+H]⁺.

### Step 2: 4-(4-amino-6-(2-fluoro-4-(2-fluoroacrylamido)phenyl)pyrazolo[5,1-f][1,2,4]triazin-5-yl)-2-methoxy-N-(2,2,2-trifluoroethyl)benzamide

A solution of 4-(4-amino-6-(4-amino-2-fluorophenyl) pyrazolo[5,1-f][1,2,4]triazin-5-yl)-2-methoxy-N-(2,2,2-trifluoroethyl)benzamide (60 mg, 0.13 mmol), 2-(trifluoromethyl) acrylic acid (27 mg, 0.20 mmol), DIPEA (32 mg, 0.26 mmol) and T3P (26 mg, 0.65 mmol) in THE (0.8 mL) was stirred at 0° C. for 5 h. The mixture was quenched with H₂O and extracted with EtOAc. The combined organic phases were washed with brine, dried over Na₂SO₄, concentrated in vacuo, and purified by silica gel chromatography column (DCM/MeOH=95:5 to 10:1), further purified by Prep-HPLC to afford the Compound 100 (8 mg, 20% yield) as a white solid. LCMS (ESI) calcd. for C₂₄H₁₈F₅N₇O₃: 547; m/z found 547 [M+H]⁺. ¹H NMR (400 Hz, DMSO, ppm) 10.56 (s, 1H), 8.67 (m, 1H), 8.21 (s, 1H), 7.76 (d, J=7.9 Hz, 1H), 7.70-7.57 (m, 2H), 7.44 (t, J=8.4 Hz, 1H), 7.11 (s, 1H), 6.92 (m, 1H), 5.74 (m, 1H), 5.48 (m, 1H), 4.17-4.05 (m, 2H), 3.79 (s, 3H).

### Example 338

### 4-(4-amino-6-(4-(2-fluoroacrylamido)phenyl)pyrazolo[5,1-f][1,2,4]triazin-5-yl)-N-(3,3-difluorocyclobutyl)benzamide (“Compound 338”)

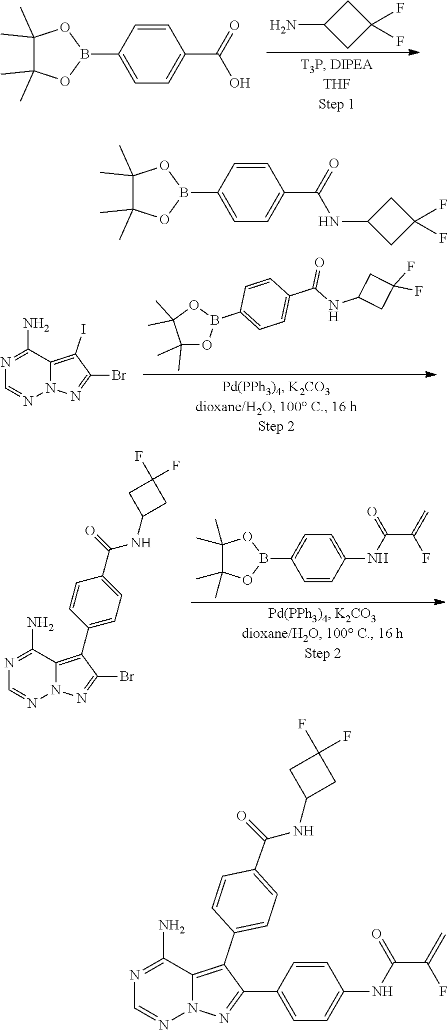

### Step 1: N-(3,3-difluorocyclobutyl)-4-(4,4,5,5-tetramethyl-1,3,2-dioxaborolan-2-yl)benzamide

To a solution of 4-(4,4,5,5-tetramethyl-1,3,2-dioxaborolan-2-yl)benzoic acid (500 mg, 2.0 mmol) and 3,3-difluorocyclobutan-1-amine (281 mg, 2.6 mmol) and DIPEA (780 mg, 6.0 mmol) in THE (10 mL), T3P (1.93 g, 3.0 mmol) was added. The reaction mixture was stirred for 3 h at room temperature. The reaction mixture was extracted by EA and H₂O, the organic layer was collected. The reaction mixture was purified by silica gel chromatography (eluting with PE/EA=5/1) to obtain the desired product (585 mg, 86% yield) as a white solid.MS (ESI): mass cacld. For C₁₇H₂₂BF₂NO₃: 337; m/z found 338 [M+1]⁺.

### Step 2: 4-(4-amino-6-bromopyrazolo[5,1-f][1,2,4]triaz in-5-yl)-N-(3,3-difluorocyclobutyl)benzamide

To a solution of N-(3,3-difluorocyclobutyl)-4-(4,4,5,5-tetramethyl-1,3,2-dioxaborolan-2-yl)benzamide (250 mg, 0.74 mmol), 6-bromo-5-iodopyrazolo[5,1-f][1,2,4]triazin-4-amine (200 mg, 0.59 mmol), K₂CO₃ (205 mg, 1.48 mmol) in dioxane (8 ml) and H₂O (1 ml), Pd(PPh₃)₄ (86 mg, 0.07 mmol) was added and the reaction system was purged with nitrogen three times. The mixture was stirred for 16 h at 100° C. The residue was purified by silica gel chromatography (eluting with DCM/MeOH=20/1) to obtain the desired product (146 mg, 47% yield) as a yellow solid. MS (ESI): mass calcd. For C₁₆H₁₃BrF₂N₆O 422, m/z found 423 [M+1]⁺.

### Step 3: 4-(4-amino-6-(4-(2-fluoroacrylamido)phenyl)pyrazolo[5,1-f][1,2,4]triazin-5-yl)-N-(3,3-difluoro cyclobutyl)benzamide

To a solution of 4-(4-amino-6-bromopyrazolo[5,1-f][1,2,4]triazin-5-yl)-N-(3,3-difluorocyclobutyl)benzamide (90 mg, 0.21 mmol), 6-bromo-5-iodopyrazolo[5,1-f][1,2,4]triazin-4-amine (93 mg, 0.32 mmol) and K₂CO₃ (59 mg, 0.43 mmol) in dioxane (5 ml) and H₂O (1 ml), Pd(PPh₃)₄ (25 mg, 0.02 mmol) was added and the reaction system was purged with nitrogen three times. The mixture was stirred for 16 h at 100° C. The residue was purified by silica gel chromatography (eluting with DCM/MeOH=20/1) to obtain the crude product. Then the crude product was purified by semi-preparative HPLC to give the Compound 338 (26.51 mg, 25% yield) as a white solid. MS (ESI): mass calcd. for C₂₅H₂₀F₃N₇O₂ 507; m/z found 508 [M+1]⁺. ¹H NMR (400 MHz, DMSO) δ 10.37 (s, 1H), 8.98 (s, 1H), 8.17 (s, 1H), 7.98 (d, J=8.0 Hz, 2H), 7.67 (d, J=8.0 Hz, 2H), 7.53 (d, J=8.0 Hz, 2H), 7.36 (d, J=8.0 Hz, 2H), 5.78-5.65 (m, 1H), 5.46-5.41 (m, 1H), 4.35-4.25 (m, 1H), 3.01-2.92 (m, 2H), 2.86-2.72 (m, 2H).

### Example 339

### N-(4-(6-(4-acrylamidophenyl)-4-aminopyrazolo[5,1-f][1,2,4]triazin-5-yl)-2-methoxyphenyl)-2,2-difluorocyclopropane-1-carboxamide (“Compound 339”)

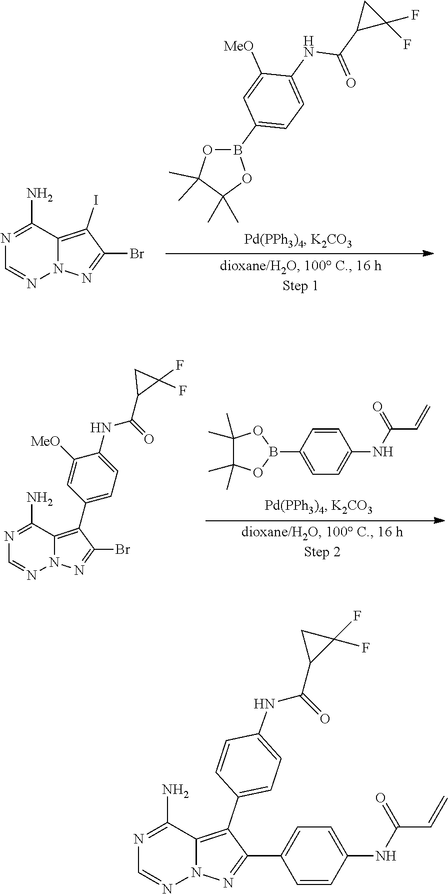 [SMILES: Nc1ncnn2nc(Br)c(I)c12 ; COc1cc(B2OC(C)(C)C(C)(C)O2)ccc1NC(=O)C1CC1(F)F ; COc1cc(-c2c(Br)nn3ncnc(N)c23)ccc1NC(=O)C1CC1(F)F ; C=CC(=O)Nc1ccc(B2OC(C)(C)C(C)(C)O2)cc1 ; C=CC(=O)Nc1ccc(-c2nn3ncnc(N)c3c2-c2ccc(NC(=O)C3CC3(F)F)cc2)cc1]

### Step 1: N-(4-(4-amino-6-bromopyrazolo[5,1-f][1,2,4]triazin-5-yl)-2-methoxyphenyl)-2,2-difluoro cyclopropane-1-carboxamide

To a solution of 2,2-difluoro-N-(2-methoxy-4-(4,4,5,5-tetramethyl-1,3,2-dioxaborolan-2-yl)phenyl)cyclopropane-1-carboxamide (187 mg, 0.53 mmol), 6-bromo-5-iodopyrazolo[5,1-f][1,2,4]triazin-4-amine (150 mg, 0.44 mmol) and K₂CO₃ (116 mg, 0.84 mmol) in dioxane (10 ml) and H₂O (3 ml), Pd(PPh₃)₄ (48 mg, 0.04 mmol) was added and the reaction system was purged with nitrogen three times. The reaction mixture was stirred for 16 h at 100° C. The reaction mixture was purified by silica gel chromatography (eluting with DCM/MeOH=10/1) to obtain the desired product (100 mg, 54% yield) as a yellow solid. MS (ESI): mass cacld. For C₁₆H₁₃BrF₂N₆O₂: 439; m/z found 440 [M+1]⁺.

### Step 2: N-(4-(6-(4-acrylamidophenyl)-4-aminopyrazolo[5,1-f][1,2,4]triazin-5-yl)-2-methoxyphenyl)-2,2-difluorocyclopropane-1-carboxamide

To a solution of N-(4-(4-amino-6-bromopyrazolo[5,1-f][1,2,4]triazin-5-yl)-2-methoxyphenyl)-2,2-difluorocyclopropane-1-carboxamide (100 mg, 0.23 mmol), N-(4-(4,4,5,5-tetramethyl-1,3,2-dioxaborolan-2-yl)phenyl)acrylamide (124 mg, 0.46 mmol), K₂CO₃ (63 mg, 0.46 mmol) in dioxane (5 ml) and H₂O (2 ml), Pd(PPh₃)₄ (23 mg, 0.02 mmol) was added and the reaction system was purged with nitrogen three times. The mixture was stirred for 16 h at 100° C. The residue was purified by prep-HPLC to obtain the desired Compound 339 (24.65 mg, 21% yield) as a white solid. MS (ESI): mass calcd. for C₂₅H₂₁F₂N₇O₃: 505; m/z found 506 [M+1]⁺. ¹H NMR (400 MHz, DMSO) δ 10.21 (s, 1H), 9.86 (s, 1H), 8.15 (d, J=7.8 Hz, 2H), 7.62 (d, J=8.7 Hz, 2H), 7.43 (d, J=8.7 Hz, 2H), 7.12 (d, J=1.4 Hz, 1H), 6.96 (d, J=8.1 Hz, 1H), 6.43 (dd, J=17.0, 10.0 Hz, 1H), 6.25 (dd, J=17.0, 1.9 Hz, 1H), 5.76 (dd, J=10.0, 2.0 Hz, 1H), 3.80 (s, 3H), 3.24 (m, 1H), 2.06-1.93 (m, 2H).

### Example 343

### N-(4-(4-amino-7-cyano-3-(3-fluoro-4-((4-methylpyrimidin-2-yl)oxy)phenyl)-1-methyl-1H-pyrrolo[3,2-c]pyridin-2-yl)phenyl)methacrylamide (“Compound 343”)

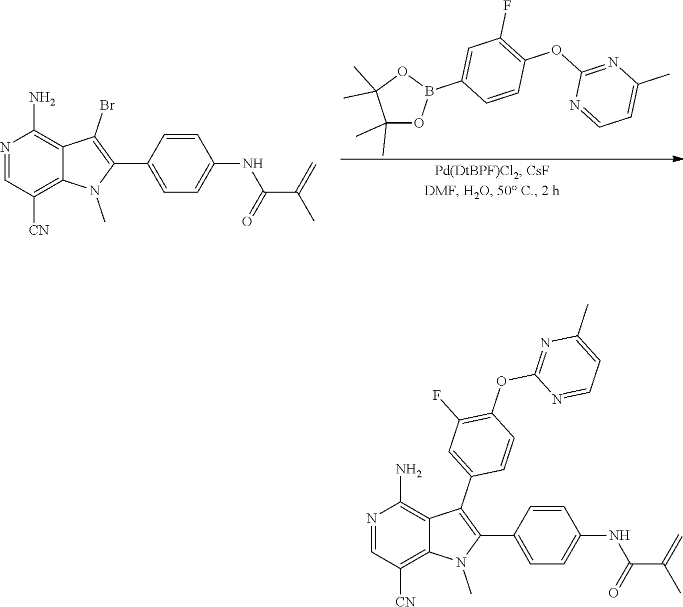

A mixture of N-(4-(4-amino-3-bromo-7-cyano-1-methyl-1H-pyrrolo[3,2-c]pyridin-2-yl)phenyl)methacryl amide (70 mg, 0.17 mmol), 2-(2-fluoro-4-(4,4,5,5-tetramethyl-1,3,2-dioxaborolan-2-yl)phenoxy)-4-methyl pyrimidine (113 mg, 0.34 mmol), Pd(DtBPF)Cl₂ (7 mg, 0.01 mmol) and CsF (52 mg, 0.34 mmol) in DMF/H₂O (3 mL/0.3 mL) was stirred at 50° C. for 2 h under N₂. The reaction mixture was concentrated in vacuo. The residue was purified by prep-HPLC to give the desired Compound 343 (14.5 mg, 17% yield) as white solid. MS (ESI): mass calcd. for C₃₀H₂₄FN₇O₂ 533.57, m/z found 534 [M+1]⁺. ¹H NMR (400 MHz, DMSO) δ 9.94 (s, 1H), 8.47 (d, J=5.0 Hz, 1H), 8.21 (s, 1H), 7.75 (d, J=8.5 Hz, 2H), 7.36 (s, 1H), 7.31 (d, J=8.5 Hz, 2H), 7.24 (dd, J=11-3, 1.8 Hz, 1H), 7.18 (d, J=5.0 Hz, 1H), 7.12 (d, J=8.2 Hz, 1H), 5.79 (s, 1H), 5.54 (s, 1H), 3.80 (s, 3H), 2.41 (s, 3H), 1.95 (s, 3H).

### Example 344

### N-(4-(4-amino-7-cyano-3-(3-fluoro-4-((4-methylpyrimidin-2-yl)oxy)phenyl)-1-methyl-1H-pyrrolo[3,2-c]pyridin-2-yl)phenyl)-2-fluoroacrylamide (“Compound 344”)

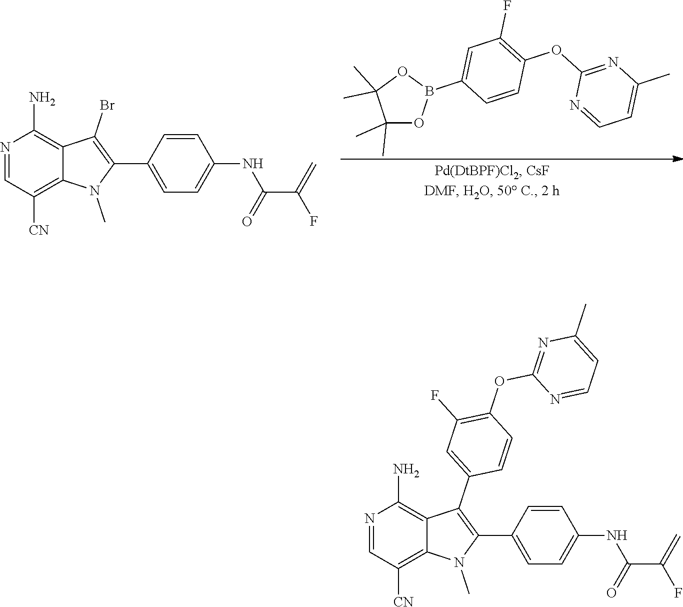

A mixture of N-(4-(4-amino-3-bromo-7-cyano-1-methyl-1H-pyrrolo[3,2-c]pyridin-2-yl)phenyl)-2-fluoroacrylamide (70 mg, 0.17 mmol), 2-(2-fluoro-4-(4,4,5,5-tetramethyl-1,3,2-dioxaborolan-2-yl)phenoxy)-4-methylpyrimidine (112 mg, 0.34 mmol), Pd(DtBPF)Cl₂ (7 mg, 0.01 mmol) and CsF (52 mg, 0.34 mmol) in DMF/H₂O (3 mL/0.3 mL) was stirred at 50° C. for 2 h under N₂. The reaction was concentrated in vacuo. The residue was purified prep-HPLC to give the desired Compound 344 (15 mg, 16% yield) as white solid. MS (ESI): mass calcd. for C₂₉H₂₁F₂N₇O₂: 537; m/z found 538 [M+1]⁺. ¹H NMR (400 MHz, DMSO) δ 10.44 (s, 1H), 8.47 (d, J=5.0 Hz, 1H), 8.21 (s, 1H), 7.80 (d, J=8.7 Hz, 2H), 7.35 (d, J=8.6 Hz, 3H), 7.25 (dd, J=11-3, 2.0 Hz, 1H), 7.19 (d, J=5.0 Hz, 1H), 7.14-7.11 (m, 1H), 5.79 (d, J=3.7 Hz, 0.5H), 5.67 (d, J=3.7 Hz, 0.5H), 5.46 (dd, J=15.6, 3.7 Hz, 1H), 3.80 (s, 3H), 2.41 (s, 3H).

### Example 346

### N-(4-(4-amino-5-(3-fluoro-4-((4-methylpyrimidin-2-yl)oxy)phenyl)pyrazolo[5,1-f][1,2,4]triazin-6-yl)phenyl)methacrylamide (“Compound 346”)

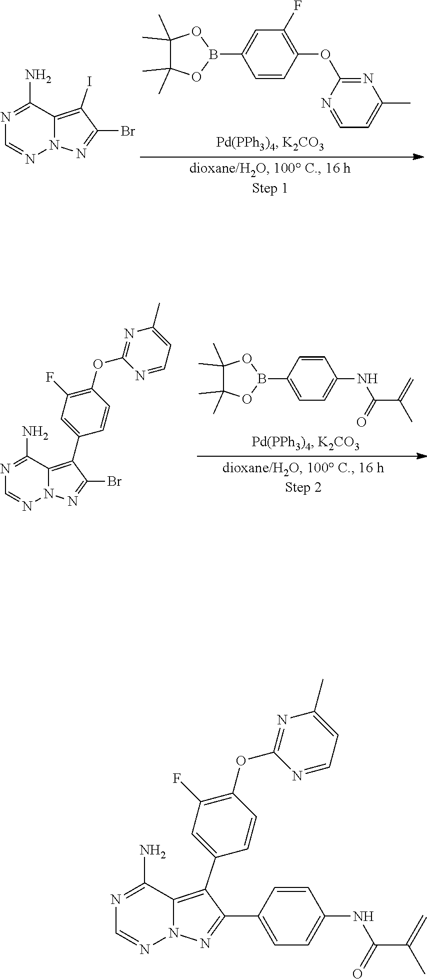

### Step 1: 6-bromo-5-(3-fluoro-4-((4-methylpyrimidin-2-yl)oxy)phenyl)pyrazolo[5,1-f][1,2,4]triazin-4-amine

To a solution of 2-(2-fluoro-4-(4,4,5,5-tetramethyl-1,3,2-dioxaborolan-2-yl)phenoxy)-4-methyl pyrimidine (293 mg, 0.89 mmol), 6-bromo-5-iodopyrazolo[5,1-f][1,2,4]triazin-4-amine (200 mg, 0.59 mmol) and K₂CO₃ (163 mg, 1.2 mmol) in dioxane (8 ml) and H₂O (2 ml), Pd(PPh₃)₄ (68 mg, 0.06 mmol) was added and the reaction system was purged with nitrogen three times. The mixture was stirred for 16 h at 100° C. The residue was purified by silica gel chromatography (eluting with DCM/MeOH=10/1) to obtain the desired product (200 mg, 82% yield) as a yellow solid. MS (ESI): mass calcd. For C₁₆H₁₁BrFN₇O: 415; m/z found 416 [M+1]⁺.

### Step 2: N-(4-(4-amino-5-(3-fluoro-4-((4-methylpyrimidin-2-yl)oxy)phenyl)pyrazolo[5,1-f][1,2,4]triazin-6-yl)phenyl)methacrylamide

To a solution of 6-bromo-5-(3-fluoro-4-((4-methylpyrimidin-2-yl)oxy)phenyl)pyrazolo[5,1-f][1,2,4]triazin-4-amine (200 mg, 0.482 mmol), N-(4-(4,4,5,5-tetramethyl-1,3,2-dioxaborolan-2-yl)phenyl)methacrylamide (208 mg, 0.723 mmol) and K₂CO₃ (134 mg, 0.048 mmol) in dioxane (8 ml) and H₂O (2 ml), Pd(PPh₃)₄ (56 mg, 0.05 mmol) was added and the reaction system was purged with nitrogen three times. The mixture was stirred for 16 h at 100° C. The residue was purified by silica gel chromatography (eluting with DCM/MeOH=10/1) to obtain the crude product. Then the crude product was purified by Prep-HPLC to give the Compound 346 (10.75 mg, 4.5% yield) as a white solid. MS (ESI): mass calcd. For C₂₆H₂₁FN₈O₂: 496; m/z found 497 [M+1]⁺. ¹H NMR (400 MHz, DMSO) δ 9.88 (s, 1H), 8.53 (d, J=4.0 Hz, 1H), 8.16 (s, 1H), 7.67 (d, J=12.0 Hz, 2H), 7.48 (m, 2H), 7.39 (d, J=12.0 Hz, 2H), 7.28 (d, J=8.0 Hz, 1H), 7.23 (d, J=4.0 Hz, 1H), 5.79 (s, 1H), 5.52 (s, 1H), 2.45 (s, 3H), 1.94 (s, 3H).

### Example 355

### 4-(2-(4-acrylamidophenyl)-4-amino-7-cyano-1-(methyl-d3)-1H-pyrrolo[3,2-c]pyridin-3-yl)-2-methoxy-N-(2,2,2-trifluoroethyl)benzamide

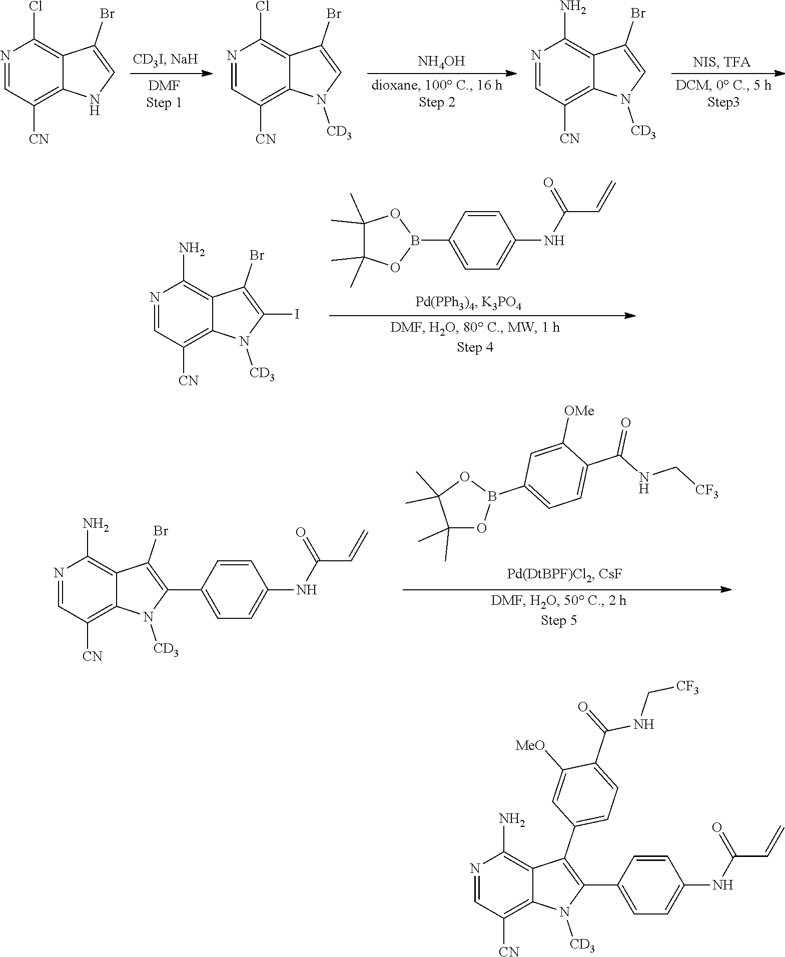

### Step 1: 3-bromo-4-chloro-1-(methyl-d3)-1H-pyrrolo[3,2-c]pyridine-7-carbonitrile

A mixture of 3-bromo-4-chloro-1H-pyrrolo[3,2-c]pyridine-7-carbonitrile (1 g, 3.8 mmol) and NaH (273 mg, 11.4 mmol) in DMF (7 mL) was stirred at 0° C. for 0.5 h under N₂, then CD₃I (1.1 g, 7.6 mmol) was added. The reaction mixture was stirred under N₂ at room temperature for 2 h. The reaction mixture was extracted with water (15 mL) and EtOAc (10 mL×3). The combined organic layers were dried over Na₂SO₄ and concentrated in vacuo. The residue was purified by FCC (PE/EtOAc=1:1) to give the desired target (800 mg, 80% yield) as yellow solid. MS (ESI): mass calcd. for C₉H₂D₃BrClN₃: 273; m/z found 274 [M+1]⁺.

### Step 2: 4-amino-3-bromo-1-(methyl-d3)-1H-pyrrolo[3,2-c]pyridine-7-carbonitrile

A mixture of 3-bromo-4-chloro-1-(methyl-d3)-1H-pyrrolo[3,2-c]pyridine-7-carbonitrile (800 mg, 2.9 mmol), NH₃·H₂O (5 mL) in 1,4-dioxane (5 mL) was stirred at 100° C. for 16 h in sealed tube. The reaction was concentrated in vacuo to give the desired target (600 mg, 80% yield) as yellow solid. MS (ESI): mass calcd. for C₉H₄D₃BrN₄: 254; m/z found 255 [M+1]⁺.

### Step 3: 4-amino-3-bromo-2-iodo-1-(methyl-d3)-1H-pyrrolo[3,2-c]pyridine-7-carbonitrile

A mixture of 4-amino-3-bromo-1-(methyl-d3)-1H-pyrrolo[3,2-c]pyridine-7-carbonitrile (650 mg, 2.56 mmol), NIS (632 mg, 2.81 mmol) and TFA (3 drops) in DMF (7 mL) was stirred at 25° C. for 16 h. The reaction mixture was extracted with water (15 mL) and EtOAc (10 mL×3). The combined organic layers were dried over Na₂SO₄ and concentrated in vacuo to give the desired target (600 mg, 62% yield) as yellow solid. MS (ESI): mass calcd. for C₉H₃D₃BrIN₄: 380; m/z found 381 [M+1]⁺.

### Step 4: N-(4-(4-amino-3-bromo-7-cyano-1-(methyl-d3)-1H-pyrrolo[3,2-c]pyridin-2-yl)phenyl) acrylamide

A mixture of 4-amino-3-bromo-2-iodo-1-(methyl-d3)-1H-pyrrolo[3,2-c]pyridine-7-carbonitrile (600 mg, 1.57 mmol), N-(4-(4,4,5,5-tetramethyl-1,3,2-dioxaborolan-2-yl)phenyl)acrylamide (431 mg, 1.57 mmol), Pd(PPh₃)₄ (181 mg, 0.15 mmol) and K₃PO₄ (450 mg, 3.14 mmol) in DMF/H₂O (6 mL/2 mL) was stirred at 80° C. for 1 h under N₂ in microwave. The reaction mixture was diluted with water (10 mL) and extracted with EtOAc (5 mL×3). The combined organic layers were dried over Na₂SO₄ and concentrated in vacuo. The residue was purified by prep-TLC (EtOAc in PE=70%) to give the desired product (400 mg, 64% yield) as yellow solid. MS (ESI): mass calcd. for C₁₈H₁₁D₃BrN₅O: 399; m/z found 400 [M+1]⁺.

### Step 5: 4-(2-(4-acrylamidophenyl)-4-amino-7-cyano-1-(methyl-d3)-1H-pyrrolo[3,2-c]pyridin-3-yl)-2-methoxy-N-(2,2,2-trifluoroethyl)benzamide

A mixture of N-(4-(4-amino-3-bromo-7-cyano-1-(methyl-d3)-1H-pyrrolo[3,2-c]pyridin-2-yl)phenyl) acrylamide (80 mg, 0.2 mmol), 2-methoxy-4-(4,4,5,5-tetramethyl-1,3,2-dioxaborolan-2-yl)-N-(2,2,2-trifluoroethyl)benzamide (144 mg, 0.4 mmol), Pd(DtBPF)Cl₂ (13 mg, 0.02 mmol) and CsF (61 mg, 0.4 mmol) in DMF/H₂O (3 mL/0.3 mL) was stirred at 50° C. for 2 h under N₂. The reaction mixture was diluted with water (10 mL) and extracted with EtOAc (5 mL×3). The combined organic layers were dried over Na₂SO₄ and concentrated in vacuo. The residue was purified by Prep-HPLC to give the desired Compound 355 (29.64 mg, 27% yield) as white solid. MS (ESI): mass calcd. for C₂₈H₂₀D₃F₃N₆O₃: 551; m/z found 552 [M+1]⁺. ¹H NMR (400 MHz, DMSO) δ 10.28 (s, 1H), 8.65 (s, 1H), 8.21 (s, 1H), 7.71-7.67 (m, 3H), 7.30 (d, J=8.6 Hz, 2H), 6.98-6.88 (m, 2H), 6.41 (d, J=10.1 Hz, 1H), 6.27 (dd, J=17.0, 1.9 Hz, 1H), 5.78 (dd, J=10.0, 2.0 Hz, 1H), 4.08 (dd, J=9.6, 6.5 Hz, 2H), 3.73 (s, 3H).

### Example 398

### N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-fluorophenyl)methacrylamide (“Compound 398”)

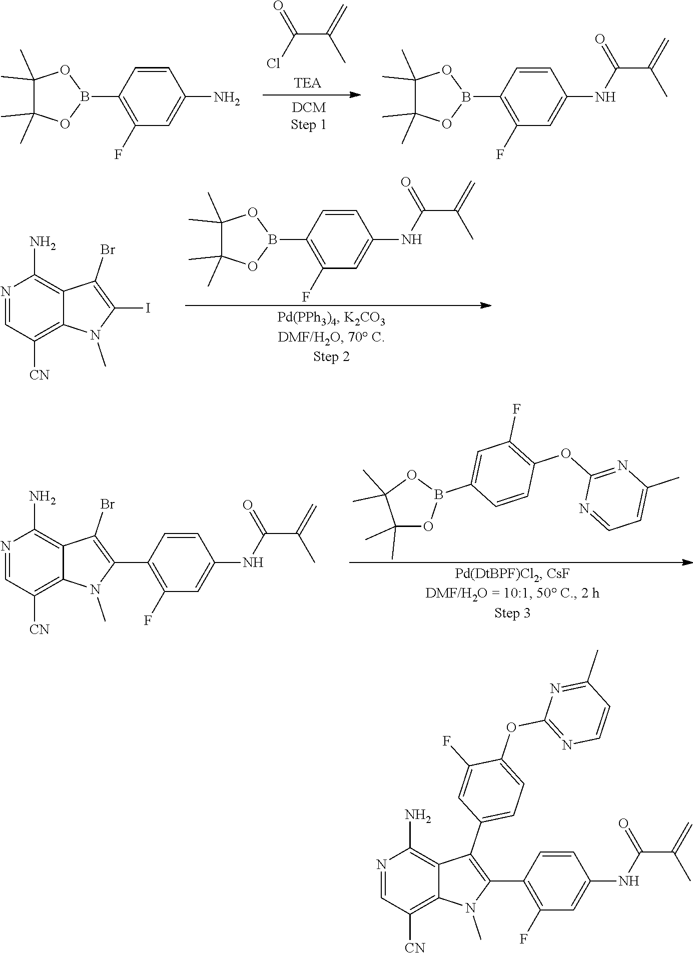

### Step 1: N-(3-fluoro-4-(4,4,5,5-tetramethyl-1,3,2-dioxaborolan-2-yl)phenyl)methacrylamide

A mixture of 3-fluoro-4-(4,4,5,5-tetramethyl-1,3,2-dioxaborolan-2-yl)aniline (3.0 g), methacryloyl chloride (1.45 g) and TEA (1.92 g) in DCM (20 mL) was stirred at 0° C. for 2 h and 20° C. for 1 h. The reaction mixture was concentrated in vacuo. The residue was purified by FCC (60 g silica gel, EtOAc in PE=20%) to give the desired target (3.1 g) as white solid. MS (ESI): mass calcd. for C₁₆H₂₁BFNO₃ 305, m/z found 306 [M+1]⁺.

### Step 2: N-(4-(4-amino-3-bromo-7-cyano-1-methyl-1H-pyrrolo[3,2-c]pyridin-2-yl)-3-fluorophenyl) methacrylamide

A mixture of N-(3-fluoro-4-(4,4,5,5-tetramethyl-1,3,2-dioxaborolan-2-yl)phenyl)methacrylamide (3.5 g), 4-amino-3-bromo-2-iodo-1-methyl-1H-pyrrolo[3,2-c]pyridine-7-carbonitrile (3.1 g), Pd(PPh₃)₄ (861 mg) and K₃PO₄ (6.0 g) in DMF/H₂O (50 mL/5 mL) was stirred at 70° C. for 10 h under N₂. The reaction mixture was quenched with water (130 mL). The precipitation was filtered to give the desired target (2.5 g) as light brown solid. MS (ESI): mass calcd. for C₁₉H₁₅BrFN₅O 427, m/z found 428 [M+1]⁺.

### Step 3: N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-fluorophenyl)methacrylamide

A mixture of N-(4-(4-amino-3-bromo-7-cyano-1-methyl-1H-pyrrolo[3,2-c]pyridin-2-yl)-3-fluorophenyl) methacrylamide (1.0 g), 2-(2-fluoro-4-(4,4,5,5-tetramethyl-1,3,2-dioxaborolan-2-yl)phenoxy)-4-methyl pyrimidine (1.16 g), Pd(DtBPF)Cl₂ (55 mg) and CsF (1.06 g) in DMF/H₂O (10 mL/1 mL) was stirred at 50° C. for 6 h under N₂. The reaction mixture was quenched with water (50 mL). The residue was filtered to give the crude mixture as light brown solid. The residue was purified by FCC (60 g silica gel, MeOH in DCM=10%), followed by Prep-HPLC to give the desired Compound 398 (508.63 mg) as white solid. MS (ESI): mass calcd. for C₃₀H₂₃F₂N₇O₂ 551, m/z found 552 [M+1]⁺. ¹H NMR (400 MHz, DMSO) δ 10.15 (s, 1H), 8.47 (d, J=5.0 Hz, 1H), 8.23 (s, 1H), 7.79 (dd, J=12.4, 1.8 Hz, 1H), 7.54 (dd, J=8.4, 1.9 Hz, 1H), 7.35 (dd, J=8.4, 4.7 Hz, 2H), 7.23 (d, J=11.5 Hz, 1H), 7.18 (d, J=5.1 Hz, 1H), 7.10 (d, J=8.0 Hz, 1H), 5.82 (s, 1H), 5.58 (s, 1H), 3.75 (s, 3H), 2.40 (s, 3H), 1.95 (s, 3H).

### Example 459

### N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-fluorophenyl)methacrylamide (“Compound 459”)

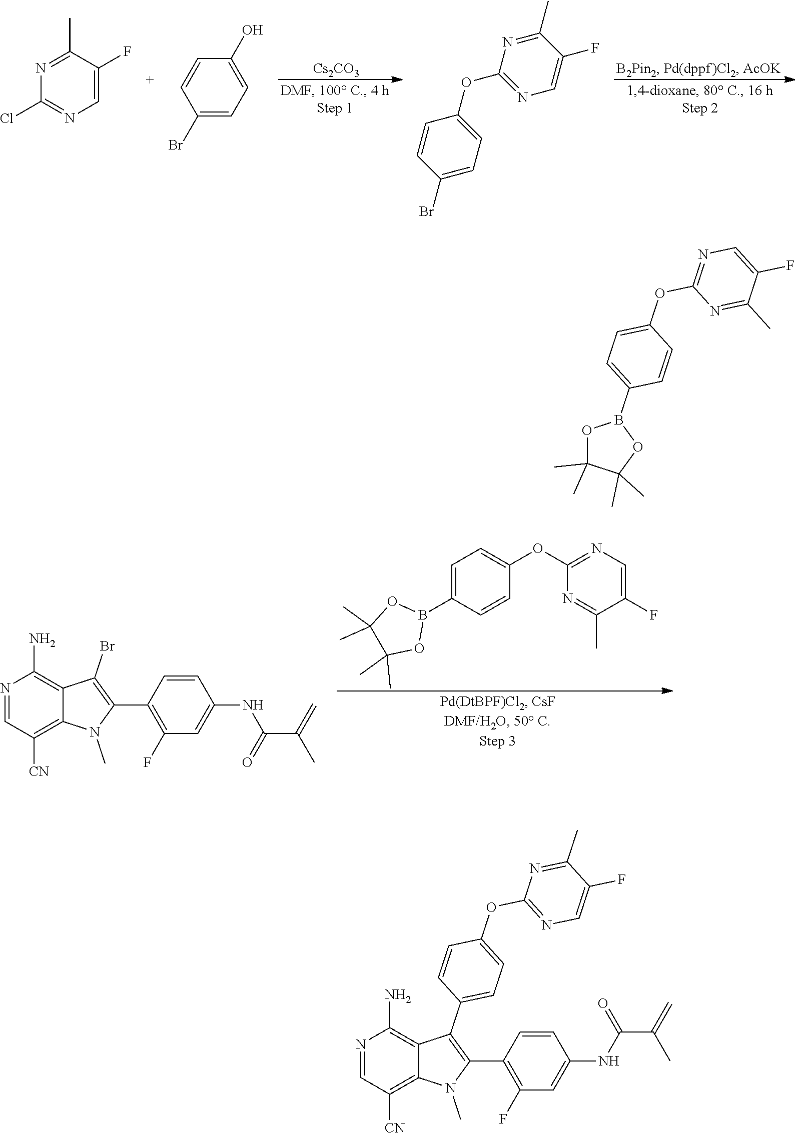

### Step 1: 2-(4-bromophenoxy)-5-fluoro-4-methylpyrimidine

To a solution of 2-chloro-5-fluoro-4-methylpyrimidine (300 mg, 2.1 mmol) and 4-bromophenol (355 mg, 3.1 mmol) in DMF (3 mL), Cs₂CO₃ (1.0 g, 3.075 mmol) was added. The reaction mixture was stirred at 100° C. for 4 h, then concentrated in vacuo. The residue was purified by gel-column chromatography (PE: EA=20:1) to afford 2-(4-bromophenoxy)-5-fluoro-4-methylpyrimidine (400 mg, 69% yield) as white oil. MS (ESI): mass calcd. C₁₁H₈BrFN₂O: 282; m/z found 283 [M+H]⁺.

### Step 2: 5-fluoro-4-methyl-2-(4-(4,4,5,5-tetramethyl-1,3,2-dioxaborolan-2-yl)phenoxy)pyrimidine

To a solution of 2-(4-bromophenoxy)-5-fluoro-4-methylpyrimidine (420 mg, 1.5 mmol), B₂Pin₂ (568 mg, 2.2 mmol) and AcOK (294 mg, 3.0 mmol) in dioxane/H₂O (20 mL), Pd(dppf)Cl₂ (54 mg, 0.075 mmol) was added. The reaction mixture was stirred at 80° C. for 16 h. The residue was purified by gel-column chromatography (PE: EA=20:1) to afford 5-fluoro-4-methyl-2-(4-(4,4,5,5-tetramethyl-1,3,2-dioxaborolan-2-yl)phenoxy)pyrimidine (200 mg, 43% yield) as white solid. MS (ESI): mass calcd. for C₁₇H₂₀BFN₂O₃: 330; m/z found 331 [M+H]⁺.

### Step 3: N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-fluorophenyl)methacrylamide

To a solution of N-(4-(4-amino-3-bromo-7-cyano-1-methyl-1H-pyrrolo[3,2-c]pyridin-2-yl)-3-fluorophenyl) methacrylamide (70 mg, 0.16 mmol), 5-fluoro-4-methyl-2-(4-(4,4,5,5-tetramethyl-1,3,2-dioxaborolan-2-yl)phenoxy)pyrimidine (89 mg, 0.27 mmol) and CsF (50 mg, 0.33 mmol) in DMF/H₂O (2.0/0.2 mL), Pd(DtBPF)Cl₂ (11 mg, 0.016 mmol) was added. The reaction mixture was stirred at 50° C. for 2 h. The mixture was concentrated and the residue was purified by gel-column chromatography (DCM: MeOH=20:1) to afford Compound 459 (10.80 mg, 8% yield) as white solid. MS (ESI): mass calcd. for C₃₀H₂₃F₂N₇O₂, 551, m/z found 552 [M+H]⁺. ¹H NMR (400 MHz, DMSO) δ 10.13 (s, 1H), 8.55 (d, J=1.2 Hz, 1H), 8.21 (s, 1H), 7.77 (m, 1H), 7.51 (m, 1H), 7.30 (t, J=8.4 Hz, 3H), 7.20 (d, J=8.8 Hz, 2H), 5.81 (s, 1H), 5.58 (s, 1H), 3.75 (s, 3H), 2.40 (d, J=2.8 Hz, 3H), 1.94 (s, 3H).

### Example 474

### N-(4-(4-amino-7-cyano-1-methyl-3-(4-((4-methylpyrimidin-2-yl)oxy)phenyl)-1H-pyrrolo[3,2-c]pyridin-2-yl)-3-chlorophenyl)methacrylamide (“Compound 474”)

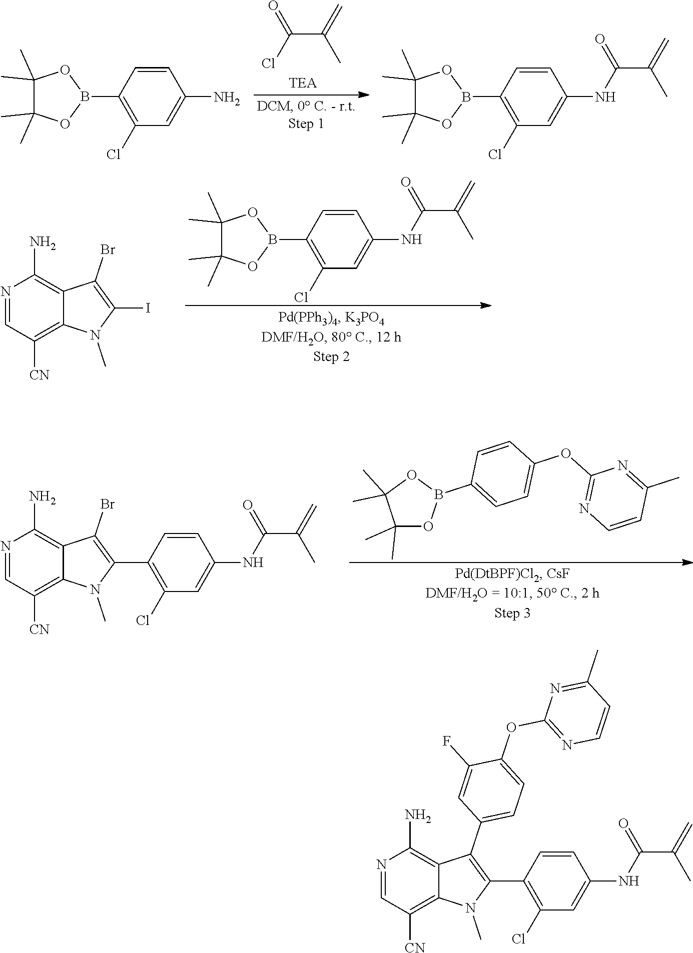

### Step 1: N-(3-chloro-4-(4,4,5,5-tetramethyl-1,3,2-dioxaborolan-2-yl)phenyl)methacrylamide

To a solution of 3-chloro-4-(4,4,5,5-tetramethyl-1,3,2-dioxaborolan-2-yl)aniline (1 g, 4 mmol) and TEA (0.81 g, 8 mmol) in DCM (10 mL), methacryloyl chloride (0.40 g, 4 mmol) was added at 0° C. The reaction mixture was stirred at room temperature for 1 h. The reaction mixture was concentrated to obtain the crude. The crude was purified by flash chromatography to give N-(3-chloro-4-(4,4,5,5-tetramethyl-1,3,2-dio-xaborolan-2-yl)phenyl) methacrylamide 1.1 g as yellow oil. MS (ESI): mass calcd. for C₁₆H₂₁BClNO₃: 321, m/z found 322 [M+H]⁺.

### Step 2: N-(4-(4-amino-3-bromo-7-cyano-1-methyl-1H-pyrrolo[3,2-c]pyridin-2-yl)-3-chlorophenyl) methacrylamide

To a solution of N-(3-chloro-4-(4,4,5,5-tetramethyl-1,3,2-dioxaborolan-2-yl)phenyl)methacrylamide (1.1 g, 3 mmol), 4-amino-3-bromo-2-iodo-1-methyl-1H-pyrrolo[3,2-c]pyridine-7-carbonitrile (1.13 g, 3 mmol), K₃PO₄ (1.27 g, 6 mmol) in DMF (20 mL) and H₂O (4 mL) stirred at 80° C. for 12 h. The reaction mixture was poured into water and filtered, collected the filter cake to obtain N-(4-(4-amino-3-bromo-7-cyano-1-methyl-1H-pyrrolo[3,2-c]pyridin-2-yl)-3-chlorophenyl)methacrylamide 800 mg as light yellow solid. MS (ESI): mass calcd. for C₁₉H₁₅BrClN₅O: 443, m/z found 444 [M+H]⁺.

### Step 3: N-(4-(4-amino-7-cyano-1-methyl-3-(4-((4-methylpyrimidin-2-yl)oxy)phenyl)-1H-pyrrolo[3,2-c]pyridin-2-yl)-3-chlorophenyl)methacrylamide

A mixture of N-(4-(4-amino-3-bromo-7-cyano-1-methyl-1H-pyrrolo[3,2-c]pyridin-2-yl)-3-chlorophenyl) methacrylamide (100 mg, 0.22 mmol), 4-methyl-2-(4-(4,4,5,5-tetramethyl-1,3,2-dioxaborolan-2-yl) phenoxy)pyrimidine (69 mg, 0.22 mmol), CsF (67 mg, 0.44 mmol) and Pd(DtBPF)Cl₂ (14 mg, 0.022 mmol) in DMF (5 mL) and H₂O (1 mL) was stirred at 50° C. for 2 h. The reaction mixture was poured into water and filtered. The filter cake was collected to obtain the crude. The crude was purified by prep-HPLC to afford Compound 474 (15.56 mg) as white solid. MS (ESI): mass calcd. for C₃₀H₂₄ClN₇O₂: 549; m/z found 550 [M+H]⁺. ¹H NMR (400 MHz, DMSO) δ 10.08 (s, 1H), 8.45 (d, J=5.0 Hz, 1H), 8.21 (s, 1H), 8.03 (d, J=2.0 Hz, 1H), 7.68 (dd, J=8.4, 2.0 Hz, 1H), 7.38 (d, J=8.4 Hz, 1H), 7.29 (d, J=7.4 Hz, 2H), 7.19 (d, J=8.8 Hz, 2H), 7.14 (d, J=5.0 Hz, 1H), 5.82 (s, 1H), 5.57 (s, 1H), 3.69 (s, 3H), 2.39 (s, 3H), 1.94 (s, 3H).

### Example 488

### N-(4-(4-amino-7-cyano-1-methyl-3-(4-((4-methylpyrimidin-2-yl)oxy)phenyl)-1H-pyrrolo[3,2-c]pyridin-2-yl)-3-methylphenyl)methacrylamide (“Compound 488”)

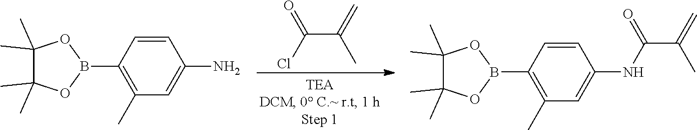

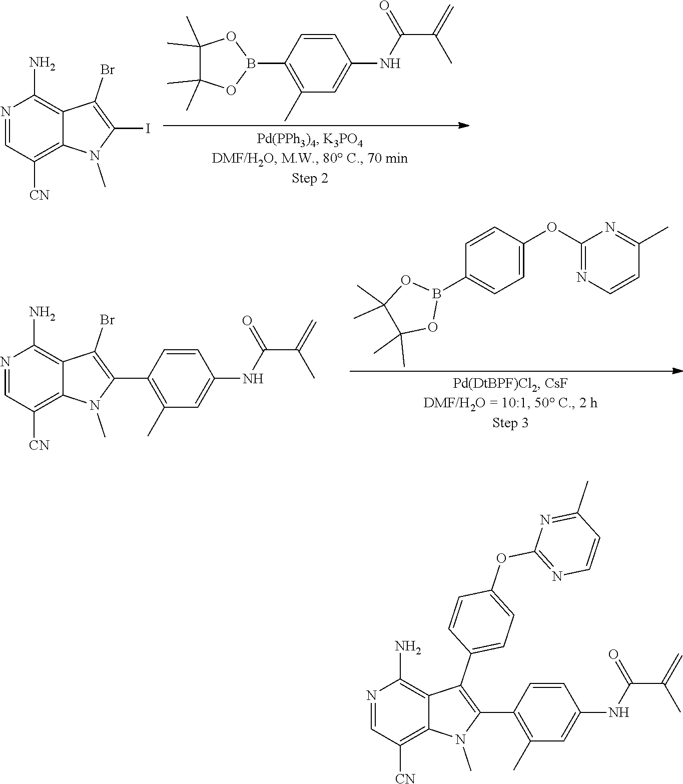

### Step 1: N-(3-methyl-4-(4,4,5,5-tetramethyl-1,3,2-dioxaborolan-2-yl)phenyl)methacrylamide

To a solution of 3-methyl-4-(4,4,5,5-tetramethyl-1,3,2-dioxaborolan-2-yl)aniline (1.6 g, 6.9 mmol) and TEA (1.0 g, 10.3 mmol) in DCM (20 mL) under N₂, methacryloyl chloride (789.0 mg, 7.5 mmol) was added dropwise at 0° C. The mixture was warmed to 25° C. and stirred for 3 h. Then water (200 ml) was added into the mixture and extracted with DCM (10 ml×3). The organic layer was dried with Na₂SO₄ and concentrated, the residue was purified by Flash chromatograph column (DCM:PE=0:100-100:0) to afford N-(3-methyl-4-(4,4,5,5-tetramethyl-1,3,2-dioxaborolan-2-yl)phenyl)methacrylamide (1.7 g, 93% yield) as yellow solid. MS (ESI): mass calcd. for C₁₇H₂₄BNO₃: 301; m/z found 302 [M+H]⁺.

### Step 2: N-(4-(4-amino-3-bromo-7-cyano-1-methyl-1H-pyrrolo[3,2-c]pyridin-2-yl)-3-methylphenyl)methacrylamide

To a solution of 4-amino-3-bromo-2-iodo-1-methyl-1H-pyrrolo[3,2-c]pyridine-7-carbonitrile (500.0 mg, 1-3 mmol) and N-(3-methyl-4-(4,4,5,5-tetramethyl-1,3,2-dioxaborolan-2-yl)phenyl) methacrylamide (479.5 mg, 1.6 mmol) in DMF/H₂O (5 mL/0.5 mL), Pd(PPh₃)₄ (153.7 mg, 0.13 mmol) and K₃PO₄ (844.5 mg, 4.0 mmol) were added. Under N₂ protection, the reaction mixture was stirred under microwave at 80° C. for 70 min. The mixture was quenched with water (50 ml) and extracted with EA (20 ml×3). The organic layer was dried over Na₂SO₄ and concentrated. The residue was purified by Flash chromatography column (MeOH:DCM=0:100-5:95) to give N-(4-(4-amino-3-bromo-7-cyano-1-methyl-1H-pyrrolo[3,2-c]pyridin-2-yl)-3-methylphenyl) methacrylamide (250 mg, 45% yield) as yellow solid. MS (ESI): mass calcd. for C₂₀H₁₈BrN₅O: 423; m/z found 424 [M+H]⁺.

### Step 3: N-(4-(4-amino-7-cyano-1-methyl-3-(4-((4-methylpyrimidin-2-yl)oxy)phenyl)-1H-pyrrolo[3,2-c]pyridin-2-yl)-3-methylphenyl)methacrylamide

To a solution of N-(4-(4-amino-3-bromo-7-cyano-1-methyl-1H-pyrrolo[3,2-c]pyridin-2-yl)-3-methylphenyl)methacrylamide (250.0 mg, 0.59 mmol) and 4-methyl-2-(4-(4,4,5,5-tetramethyl-1,3,2-dioxaborolan-2-yl)phenoxy)pyrimidine (183.9 mg, 0.59 mmol) in DMF/H₂O (2 mL/0.2 mL), Pd(dtbpf)Cl₂ (38.1 mg, 0.059 mmol) and CsF (178.9 mg, 1.18 mmol) were added. The reaction mixture was stirred at 50° C. for 2 h under N₂ protection. 20 ml of water was added into the mixture and extracted with EA (10 ml×3). The organic layer was dried over Na₂SO₄ and concentrated. The residue was purified by pre-HPLC to give Compound 488 (22.5 mg) as white solid. MS (ESI): mass calcd. for C₃₁H₂₇N₇O₂: 529; m/z found 530 [M+H]⁺. ¹H NMR (400 MHz, DMSO) δ 9.86 (s, 1H), 8.45 (d, J=5.2 Hz, 1H), 8.37 (s, 1H), 7.65 (d, J=1.6 Hz, 1H), 7.60 (dd, J=8.4, 2.0 Hz, 1H), 7.30-7.23 (m, 3H), 7.18 (d, J=8.8 Hz, 3H), 7.15 (d, J=5.2 Hz, 1H), 5.79 (s, 1H), 5.53 (s, 1H), 3.69 (s, 3H), 2.39 (s, 3H), 1.98 (s, 3H), 1.94 (s, 3H).

### Example 499

### N-(4-(4-amino-7-cyano-3-(3-fluoro-4-((1-methyl-1H-pyrazol-3-yl)oxy)phenyl)-1-methyl-1H-pyrrolo[3,2-c]pyridin-2-yl)-3-fluorophenyl)methacrylamide (“Compound 499”)

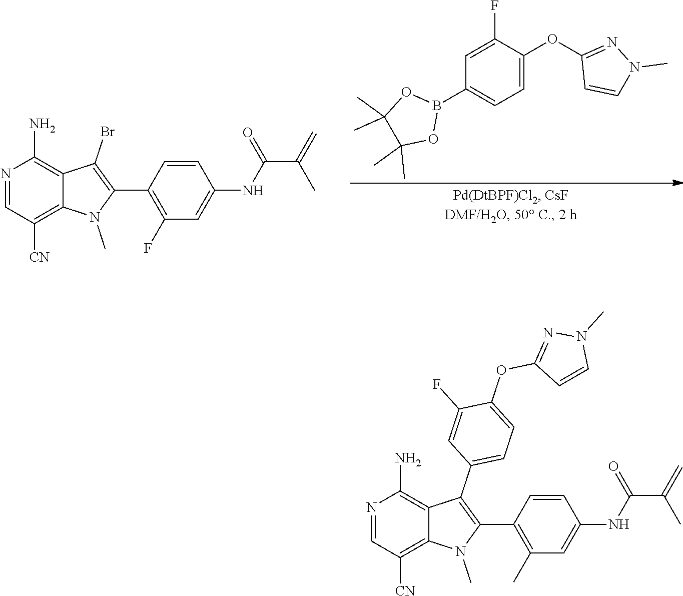

To a solution of N-(4-(4-amino-3-bromo-7-cyano-1-methyl-1H-pyrrolo[3,2-c]pyridin-2-yl)-3-fluorophenyl)methacrylamide (80 mg, 0.19 mmol), 3-(2-fluoro-4-(4,4,5,5-tetramethyl-1,3,2-dioxaborolan-2-yl)phenoxy)-1-methyl-1H-pyrazole (89 mg, 0.29 mmol) and CsF (90 mg, 0.57 mmol) in dioxane (8 ml) and H₂O (2 ml), Pd(DtBPF)Cl₂ (24 mg, 0.02 mmol) was added. The reaction mixture was stirred at 50° C. for 1 h. The residue was purified by silica gel chromatography (eluting with DCM/MeOH=10/1) to give the crude product, then purified by Prep-HPLC to give Compound 499 (27.91 mg, 34.2% yield) as a white solid. MS (ESI): mass calcd. For C₂₅H₂₀FN₉O: 539; m/z found 540 [M+1]⁺. ¹H NMR (400 MHz, DMSO) δ 10.13 (s, 1H), 8.21 (s, 1H), 7.76 (dd, J=12.4, 1.8 Hz, 1H), 7.63 (d, J=2.4 Hz, 1H), 7.51 (dd, J=8.4, 2.0 Hz, 1H), 7.29 (t, J=8.4 Hz, 1H), 7.16 (t, J=8.6 Hz, 2H), 6.99 (d, J=8.0 Hz, 1H), 5.88-5.80 (m, 2H), 5.58 (s, 1H), 3.74 (s, 3H), 3.72 (s, 3H), 1.95 (s, 3H).

### Example 501

### N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-methylphenyl)methacrylamide (“Compound 501”)

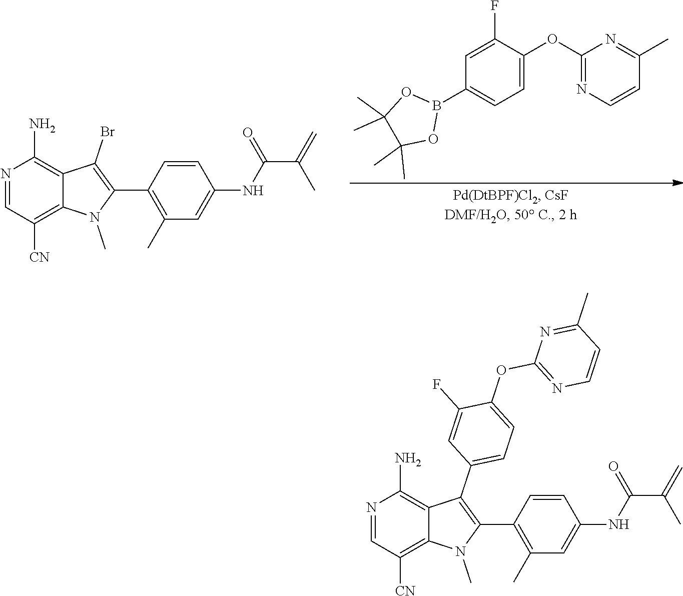

To a solution of N-(4-(4-amino-3-bromo-7-cyano-1-methyl-1H-pyrrolo[3,2-c]pyridin-2-yl)-3-methylphenyl)methacrylamide (190.0 mg, 0.45 mmol) and 2-(2-fluoro-4-(4,4,5,5-tetramethyl-1,3,2-dioxaborolan-2-yl)phenoxy)-4-methylpyrimidine (147.9 mg, 0.45 mmol) in DMF/H₂O (2 mL/0.2 mL), Pd(dtbpf)Cl₂ (29.1 mg, 0.045 mmol) and CsF (136.1 mg, 0.90 mmol) were added. The reaction mixture was stirred at 50° C. for 2 h under N₂ protection. 20 ml of water was added into the mixture and extracted with EA (10 ml×3). The organic layer was dried over Na₂SO₄ and concentrated. The residue was purified by pre-HPLC to give Compound 501 (26.0 mg) as white solid. MS (ESI): mass calcd. for C₃₁H₂₆FN₇O₂: 547; m/z found 548 [M+H]⁺. ¹H NMR (400 MHz, DMSO) δ 9.89 (s, 1H), 8.46 (d, J=5.2 Hz, 1H), 8.39 (s, 1H), 7.67 (s, 1H), 7.65-7.58 (m, 1H), 7.35 (t, J=8.4 Hz, 1H), 7.27 (d, J=8.4 Hz, 1H), 7.21 (dd, J=11.6, 2.0 Hz, 1H), 7.18 (d, J=5.2 Hz, 1H), 7.07 (d, J=10.0 Hz, 1H), 5.80 (s, 1H), 5.53 (s, 1H), 3.70 (s, 3H), 2.40 (s, 4H), 1.98 (s, 3H), 1.94 (s, 3H).

### Example 513

### N-(4-(4-amino-7-cyano-1-methyl-3-(4-((1-methyl-1H-pyrazol-3-yl)oxy)phenyl)-1H-pyrrolo[3,2-c]pyridin-2-yl)-3-chlorophenyl)methacrylamide (“Compound 513”)

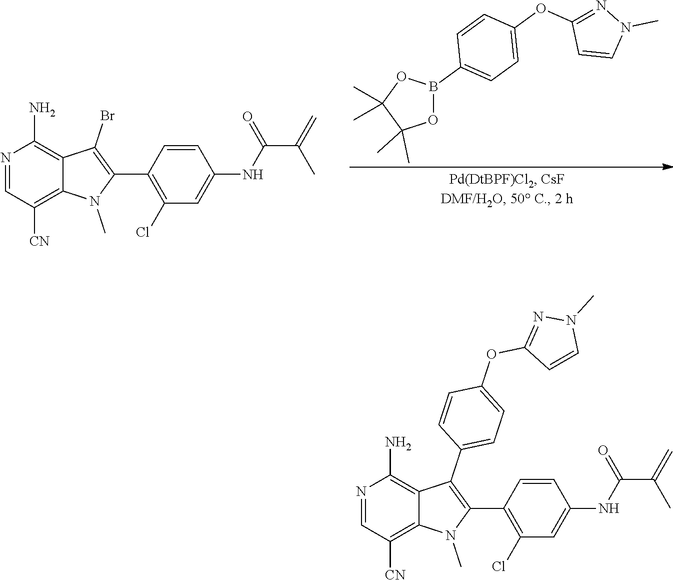

A mixture of N-(4-(4-amino-3-bromo-7-cyano-1-methyl-1H-pyrrolo[3,2-c]pyridin-2-yl)-3-chlorophenyl) methacryl amide (100 mg, 0.22 mmol), 1-methyl-3-(4-(4,4,5,5-tetramethyl-1,3,2-dioxaborolan-2-yl)phenoxy)-1H-pyrazole (98 mg, 0.22 mol), CsF (71 mg, 0.44 mmol) and Pd(DtBPF)Cl₂ (14 g, 0.022 mmol) in DMF (10 mL) and H₂O (1 mL) was stirred under N₂ at 50° C. for 2 h. The reaction mixture was concentrated in vacuo. The residue was purified by prep-HPLC to give Compound 513 (12.31 mg) as white solid. MS (ESI): mass calcd. for C₂₉H₂₄ClN₇O₂: 537, m/z found 538 [M+H]⁺. ¹H NMR (400 MHz, DMSO) δ 10.08 (s, 1H), 8.20 (s, 1H), 8.01 (d, J=2.0 Hz, 1H), 7.64 (d, J=2.3 Hz, 2H), 7.33 (d, J=8.4 Hz, 1H), 7.20 (d, J=6.9 Hz, 2H), 7.02 (d, J=8.8 Hz, 2H), 5.88 (d, J=2.3 Hz, 1H), 5.82 (s, 1H), 5.58 (s, 1H), 3.74 (s, 3H), 3.68 (s, 3H), 1.94 (s, 3H).

### Example 507

### N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)phenyl)acrylamide (“Compound 507”)

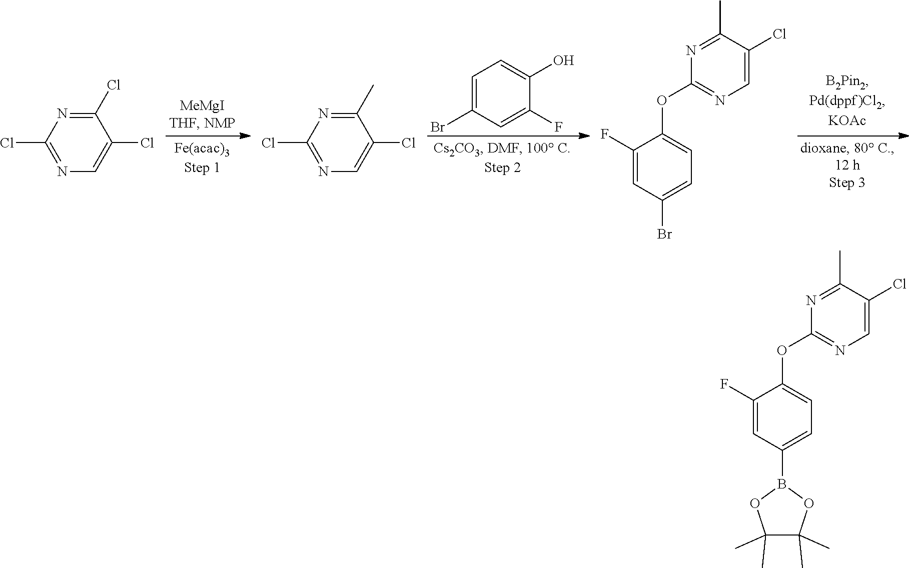

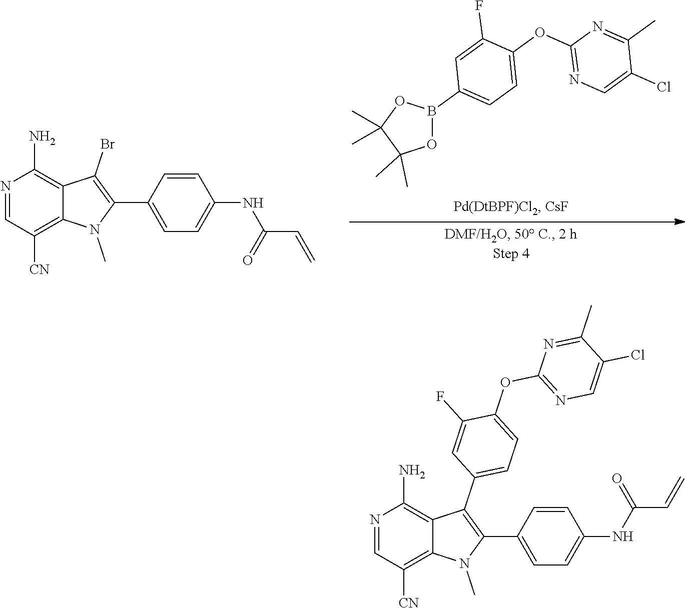 [SMILES: C=CC(=O)Nc1ccc(-c2c(Br)c3c(N)ncc(C#N)c3n2C)cc1 ; Cc1nc(Oc2ccc(B3OC(C)(C)C(C)(C)O3)cc2F)ncc1Cl ; C=CC(=O)Nc1ccc(-c2c(-c3ccc(Oc4ncc(Cl)c(C)n4)c(F)c3)c3c(N)ncc(C#N)c3n2C)cc1]

### Step 1: 2,5-dichloro-4-methylpyrimidine

To a solution of 2,4,5-trichloropyrimidine (3.0 g, 16.5 mmol) and Fe(acac)₃ (1.16 g, 3.3 mmol) in NMP (3 ml) and THF (18 mL) was added methyl magnesium iodide (5.5 g, 33.0 mmol). This mixture was stirred for 12 h at 25° C. The residue was extracted by EA and H₂O. The organic layer was collected and concentrated in vacuo. The residue was purified by silica gel chromatography (eluting with PE/EA=20/1) to obtain the desired product (1.1 g, 41% yield) as light yellow oil. MS (ESI): mass calcd. for C₅H₄Cl₂N₂: 162; m/z found 163 [M+1]⁺.

### Step 2: 2-(4-bromo-2-fluorophenoxy)-5-chloro-4-methylpyrimidine

To a solution of 2,5-dichloro-4-methylpyrimidine (1.1 g, 6.9 mmol) and Cs2CO₃ (4.43 g, 13.6 mmol) in DMF (25 ml) was added 4-bromo-2-fluorophenol (1.42 g, 7.5 mmol). The mixture was stirred for 12 h at 100° C. The residue was purified by silica gel chromatography (eluting with PE/EA=20/1) to obtain the desired product (1-3 g, 61% yield) as yellow oil. MS (ESI): mass calcd. for C₁₁H₇BrClFN₂O 316; m/z found 317 [M+1]⁺.

### Step 3: 5-chloro-2-(2-fluoro-4-(4,4,5,5-tetramethyl-1,3,2-dioxaborolan-2-yl)phenoxy)-4-methylpyrimidine

To a solution of 2-(4-bromo-2-fluorophenoxy)-5-chloro-4-methylpyrimidine (1-3 g, 4.1 mmol), B₂Pin₂ (1.57 g, 6.2 mmol) and KOAc (806 mg, 8.2 mmol) in dioxane (25 ml) was added Pd(dppf)Cl₂ (343 mg, 0.42 mmol) and purged with nitrogen three times. The mixture was stirred for 16 h at 80° C. The residue was purified by silica gel chromatography (eluting with PE/EA=20/1) to obtain the desired product (1.25 g, 84% yield) as light green solid. MS (ESI): mass calcd. For C₁₇H₁₉BClFN₂O₃: 364; m/z found 365 [M+1]⁺.

### Step 4: N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)phenyl)acrylamide

To a solution of 5-chloro-2-(2-fluoro-4-(4,4,5,5-tetramethyl-1,3,2-dioxaborolan-2-yl)phenoxy)-4-methylpyrimidine (150 mg, 0.40 mmol), N-(4-(4-amino-3-bromo-7-cyano-1-methyl-1H-pyrrolo[3,2-c]pyridin-2-yl)phenyl)acrylamide (80 mg, 0.20 mmol) and CsF (61 mg, 0.40 mmol) in DMF (5 ml) and H₂O (0.5 ml) was added Pd(DtBPF)Cl₂ (13 mg, 0.02 mmol). The reaction mixture was stirred for 2 h at 50° C. The residue was purified by silica gel chromatography (eluting with DCM/MeOH=10/1) to give the crude product, then purified by Prep-HPLC to give Compound 507 (23.36 mg, 21% yield) as a white solid. MS (ESI): mass calcd. For C₂₉H₂₁ClFN₇O₂: 553; m/z found 554 [M+1]⁺. ¹H NMR (400 MHz, DMSO) δ 10.34 (s, 1H), 8.63 (s, 1H), 8.42 (s, 1H), 7.76-7.74 (d, J=8.0 Hz, 2H), 7.42-7.38 (t, J=8.0 Hz, 1H), 7.33-7.31 (d, J=8.0 Hz, 2H), 7.30-7.27 (m, 1H), 7.13-7.11 (m, 1H), 6.48-6.41 (m, 1H), 6.30-6.26 (m, 1H), 5.81-5.78 (m, 1H), 3.85 (s, 3H), 2.47 (s, 3H).

### Example 532

### 4-(3-acrylamido-12-amino-9-cyano-6,7-dihydrobenzo[f]pyrido[3′,4′:4,5]pyrrolo[1,2-d][1,4]oxazepin-13-yl)-N-(3,3-difluorocyclobutyl)-2-methoxybenzamide (“Compound 532”)

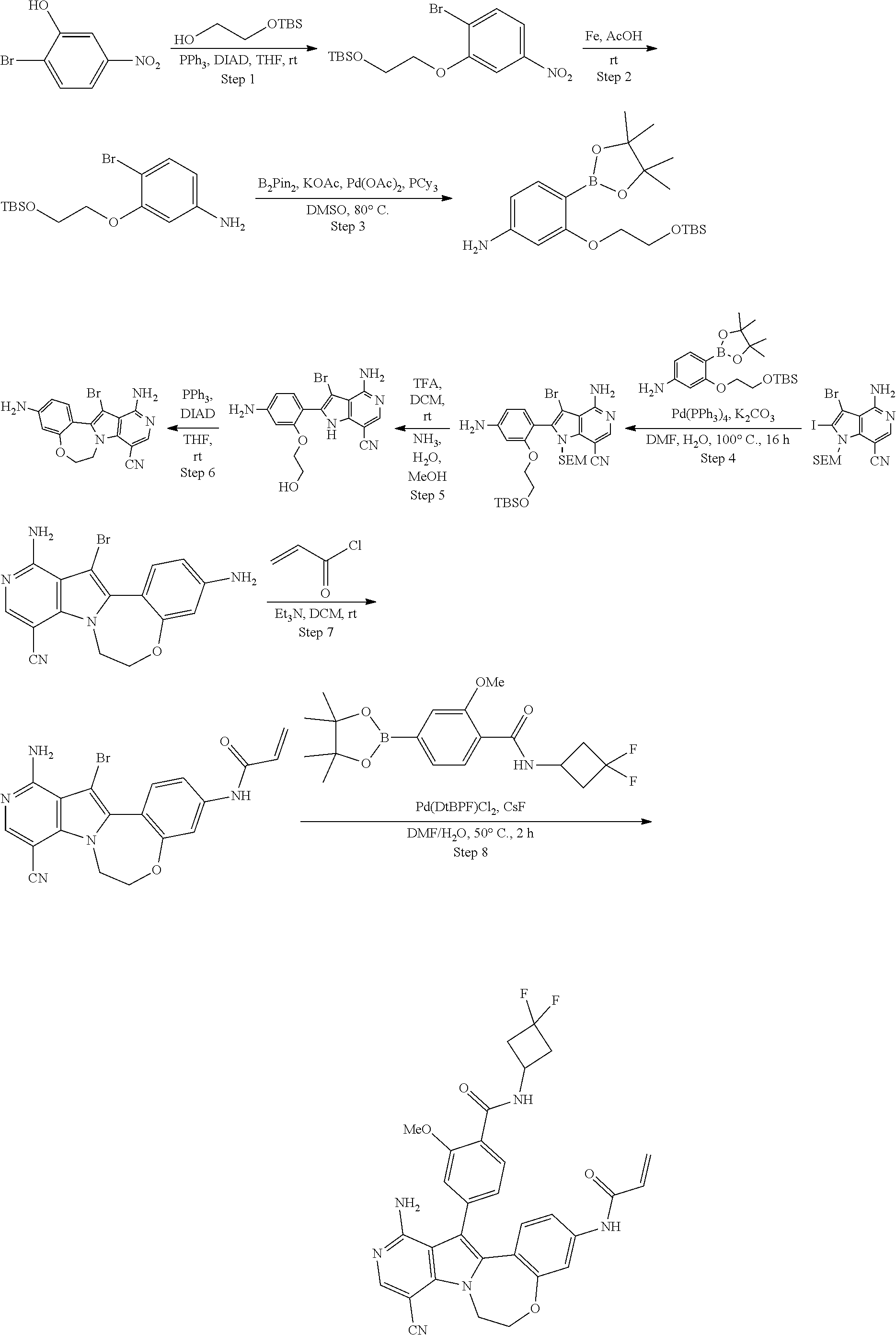

### Step 1: (2-(2-bromo-5-nitrophenoxy) ethoxy) (tert-butyl)dimethylsilane

To a solution of 2-bromo-5-nitrophenol (2 g, 9.17 mmol, 1.0 eq), 2-((tert-butyldimethylsilyl)oxy)ethan-1-ol (1.94 g, 11 mmol, 1.2 eq), PPh₃ (3.6 g, 13.8 mmol, 1.5 eq) in THE (40 mL) was added DIAD (2.78 g, 13.8 mmol, 1.5 eq) at 0° C. under N₂ atmosphere. The mixture was stirred at r.t. for 16 h. The reaction mixture was concentrated and purified by silica gel chromatography column (PE:EtOAc=5:1) (3 g, 80% yield) as a yellow oil. LCMS (ESI) calcd. for C₁₄H₂₂BrNO₄Si: 375; found 376 [M+1]⁺.

### Step 2: 4-bromo-3-(2-((tert-butyldimethylsilyl)oxy)ethoxy)aniline

To a solution of (2-(2-bromo-5-nitrophenoxy) ethoxy) (tert-butyl)dimethylsilane (3.0 g, 7.98 mmol, 1.0 eq) in AcOH (40 mL) was added Fe (4.5 g, 79.8 mmol, 10 eq). The reaction mixture was stirred at r.t. for 2 h. The mixture was concentrated and purified by silica gel chromatography column (PE:EtOAc=3:1) (2 g, crude) as a yellow oil. LCMS (ESI) calcd. for C₁₄H₂₄BrNO₂Si: 345; found 346 [M+1]⁺.

### Step 3: 3-(2-((tert-butyldimethylsilyl)oxy) ethoxy)-4-(4,4,5,5-tetramethyl-1,3,2 dioxaborolan-2-yl) aniline

To a solution of 4-bromo-3-(2-((tert-butyldimethylsilyl)oxy)ethoxy)aniline (1.80 g, 5.2 mmol, 1.0 eq), B₂Pin₂ (1.98 g, 7.8 mmol, 1.5 eq), KOAc (1.02 g, 0.54 mmol, 2.0 eq) in DMSO (15 mL) was added Pd(OAc)₂ (238 mg, 1.04 mmol, 0.2 eq) and PCy₃ (582 mg, 2.08 mmol, 0.4 eq). The reaction mixture was stirred at 50° C. for 16 h under N₂ protection. The mixture was quenched with H₂O and extracted with EtOAc. The combined organic phases were washed with brine, dried over Na₂SO₄, concentrated in vacuo, and purified by FCC (DCM: MeOH=20:1) to afford the product (800 mg) as black solid. LCMS (ESI) calcd. for C₂₀H₃₆BNO₄Si: 393; found 394 [M+1].

### Step 4: 4-(4-amino-6-(4-(2-fluoroacrylamido)-2-methoxyphenyl)pyrazolo[5,1-f][1,2,4]triazin-5-yl)-N-cyclopropyl-2-methoxybenzamide

To a solution of 3-(2-((tert-butyldimethylsilyl)oxy)ethoxy)-4-(4,4,5,5-tetramethyl-1,3,2-dioxaborolan-2-yl)aniline (1-3 g, 3.3 mmol, 1-35 eq), 4-amino-3-bromo-2-iodo-1-((2-(trimethylsilyl)ethoxy)methyl)-1H-pyrrolo[3,2-c]pyridine-7-carbonitrile (1.22 g, 2.47 mmol, 1.0 eq) in DMF/H₂O (15/1.5 mL) was added K₂CO₃ (680 mg, 4.94 mmol, 2 eq.) and Pd(PPh₃)₄ (285 mg, 0.25 mmol, 0.05 eq), the reaction mixture was stirred at 100° C. for 16 h under N₂ atmosphere.

The mixture was quenched with H₂O and extracted with EtOAc. The combined organic phases were washed with brine, dried over Na₂SO₄, concentrated in vacuo, and purified by FCC to give the product (1 g, 80% purity). LCMS (ESI) calcd. for C₂₈H₄₂BrN₅O₃Si₂ 631, found 632 [M+1]⁺.

### Step 5: 4-amino-2-(4-amino-2-(2-hydroxyethoxy)phenyl)-3-bromo-1H-pyrrolo[3,2-c]pyridine-7-carbonitrile

To a solution of 4-(4-amino-6-(4-(2-fluoroacrylamido)-2-methoxyphenyl)pyrazolo[5,1-f][1,2,4]triazin-5-yl)-N-cyclopropyl-2-methoxybenzamide (1 g, 1.58 mmol, 1.0 eq) in DCM (15 mL) was added TFA (4 mL). The reaction mixture was stirred at 25° C. for 4 h, then concentrated. The residue was redissolved in MeOH/NH₄OH (10 mL/2 mL), then stirred at r.t. for 1 h. The reaction mixture was quenched with H₂O (50 mL) and filtered to give the product (260 mg) as grey solid. LCMS (ESI) calcd. for C₁₆H₁₄BrN₅O₂ 387, found 388 [M+1]⁺.

### Step 6: 3,12-diamino-13-bromo-6,7-dihydrobenzo[f]pyrido[3′,4′:4,5]pyrrolo[1,2-d][1,4]oxazepine-9-carbonitrile

To a solution of 4-amino-2-(4-amino-2-(2-hydroxyethoxy)phenyl)-3-bromo-1H-pyrrolo[3,2-c]pyridine-7-carbonitrile (120 mg, 0.31 mmol, 1.0 eq) and PPh₃ (240 mg, 0.92 mmol) in THE (20 mL) under the protection of N₂, DIAD (280 mg, 1-39 mmol) was added at 0° C. The reaction mixture was stirred at r.t. for 24 h, then concentrated in vacuo. The residue was purified by FCC (DCM: MeOH=20:1) to give the product (200 mg, crude) as yellow oil. LCMS (ESI) calcd. for C₁₆H₁₂BrNsO: 369; found 370 [M+1]⁺.

### Step 7: N-(12-amino-13-bromo-9-cyano-6,7-dihydrobenzo[f]pyrido[3′,4′:4,5]pyrrolo[1,2-d][1,4]oxazepin-3-yl)acrylamide

To a solution of 3,12-diamino-13-bromo-6,7-dihydrobenzo[f]pyrido[3′,4′:4,5]pyrrolo[1,2-d][1,4]oxazepine-9-carbonitrile (100 mg, 0.27 mmol, 1.0 eq) in DCM (8 mL) under the protection of N₂, acryloyl chloride (74 mg, 0.81 mmol) was added at −20° C. The reaction mixture was stirred at −20° C. for 30 min, then concentrated in vacuo. The residue was purified by FCC (DCM: MeOH=20:1) to give the product (50 mg, crude) as white solid. LCMS (ESI) calcd. for C₁₉H₁₄BrNsO₂: 423; found 424 [M+1]⁺.

### Step 8: 4-(3-acrylamido-12-amino-9-cyano-6,7-dihydrobenzo[f]pyrido[3′,4′:4,5]pyrrolo[1,2-d][1,4]oxazepin-13-yl)-N-(3,3-difluorocyclobutyl)-2-methoxybenzamide

To a solution of N-(12-amino-13-bromo-9-cyano-6,7-dihydrobenzo[f]pyrido[3′,4′:4,5]pyrrolo[1,2-d][1,4]oxazepin-3-yl)acrylamide (45 mg, 0.11 mmol, 1.0 eq) and N-(3,3-difluorocyclobutyl)-2-methoxy-4-(4,4,5,5-tetramethyl-1,3,2-dioxaborolan-2-yl)benzamide (60 mg, 0.16 mmol, 1.5 eq) in DMF/H₂O (5/0.5 mL) was added CsF (32 mg, 0.22 mmol, 2.0 eq) and Pd(DtBPF)Cl₂ (7 mg, 0.01 mmol, 0.1 eq). The reaction mixture was stirred at 50° C. for 2 h under N₂, then concentrated. The residue was purified by FCC (DCM: MeOH=20:1) and Prep-HPLC to give the Compound 532 (5 mg) as white solid. LCMS (ESI) calcd. for C₃₁H₂₆F₂N₆O₄: 584; found 585 [M+1]⁺. ¹H NMR (400 MHz, DMSO) δ 10.32 (s, 1H), 8.57 (d, J=6.8 Hz, 1H), 8.22 (s, 1H), 7.69 (m, 2H), 7.24-7.22 (m, 1H), 7.07 (s, 1H), 6.97 (d, J=8.0 Hz, 1H), 6.86 (d, J=8.4 Hz, 1H), 6.44-6.38 (m, 1H), 6.30-6.24 (m, 1H), 5.80-5.77 (m, 1H), 4.59 (s, 4H), 4.30-4.26 (m, 1H), 3.79 (s, 3H), 3.00-2.89 (m, 2H), 2.82-2.70 (m, 2H).

Examples of compounds of the present invention include those listed in the Table 2 and exemplification herein, or a pharmaceutically acceptable salt, prodrug, solvate, hydrate, tautomer, and isomer thereof. Some of those compounds can be prepared according to the similar method of Example 1, 71,100,338, 339, or 346; and some of those compounds can be prepared according to the similar method of Example 28,343,344, 355, 398, 459, 474, 488, 499, 501, or 513. Also some of those compounds can be prepared with reference to the above examples or combinations thereof.

TABLE 2

| Cpds | | | LC-MS |
|---|---|---|---|
| ID | Name | ¹H NMR | [M + H]⁺ |
| 2 | N-(4-(3-(3-methoxy-4- | ¹H NMR (400 MHz, DMSO) δ 10.24 (s, 1H), | 479 |
| | ((4-methylpyrimidin-2- | 8.52-8.51 (m, 1H), 8.46 (d, J = 5.2 Hz, 1H), 8.20 | |
| | yl)oxy)phenyl)imidazo[1,2- | (m, 1H), 7.73-7.59 (m, 4H), 7.39-7.25 (m, | |
| | b]pyridazin-2- | 3H), 7.16 (m, 2H), 6.44 (m, 1H), 6.26 (m, 1H), | |
| | yl)phenyl)acrylamide | 5.76 (m, 1H), 3.62 (s, 3H), 2.44 (s, 3H). | |
| 3 | N-(4-(9-(3-methoxy-4- | ¹H NMR (400 MHz, DMSO) δ 10.39 (s, 1H), | 480 |
| | ((4-methylpyrimidin-2- | 9.26 (s, 1H), 8.92 (s, 1H), 8.47 (d, J = 5.0 Hz, | |
| | yl)oxy)phenyl)-9H- | 1H), 7.74 (d, J = 8.8 Hz, 2H), 7.62 (d, J = 8.8 | |
| | purin-8- | Hz, 2H), 7.37 (dd, J = 14.9, 5.3 Hz, 2H), 7.16 (d, | |
| | yl)phenyl)acrylamide | J = 5.0 Hz, 1H), 7.08 (dd, J = 8.4, 2.3 Hz, 1H), | |
| | | 6.45 (dd, J = 17.0, 10.1 Hz, 1H), 6.29 (dd, J = | |
| | | 17.0, 2.0 Hz, 1H), 5.80 (dd, J = 10.1, 2.0 Hz, | |
| | | 1H), 3.61 (s, 3H), 2.43 (s, 3H). | |
| 4 | N-(4-(7-(3-methoxy-4- | ¹H NMR (400 MHz, DMSO) δ 10.29 (s, 1H), | 480 |
| | ((4-methylpyrimidin-2- | 8.64 (m, 2H), 8.46 (d, J = 5.2 Hz, 1H), 7.72 (s, | |
| | yl)oxy)phenyl)imidazo[1,2- | 4H), 7.36 (m, 2H), 7.26-7.06 (m, 2H), 6.45 (m, | |
| | b][1,2,4]triazin-6- | 1H), 6.27 (m, 1H), 5.77 (m, 1H), 3.62 (s, 3H), | |
| | yl)phenyl)acrylamide | 2.44 (s, 3H). | |
| 5 | N-(4-(4-amino-3-(3- | ¹H NMR (400 MHz, DMSO) δ 10.34 (s, 1H), | 495 |
| | methoxy-4-((4- | 8.44 (d, J = 5.2 Hz, 1H), 8.38 (s, 1H), 8.21-8.13 | |
| | methylpyrimidin-2- | (m, 2H), 7.86 (d, J = 9.2 Hz, 2H), 7.49 (s, 1H), | |
| | yl)oxy)phenyl)-1H- | 7.37 (d, J = 0.8 Hz, 2H), 7.14 (d, J = 5.2 Hz, | |
| | pyrazolo[3,4- | 1H), 6.47 (m, 1H), 6.29 (dd, J = 17.0, 2.0 Hz, | |
| | d]pyrimidin-1- | 1H), 5.79 (dd, J = 10.1, 2.0 Hz, 1H), 3.79 (s, | |
| | yl)phenyl)acrylamide | 3H), 2.42 (s, 3H). | |
| 6 | N-(4-(5-amino-3-(3- | ¹H NMR (400 MHz, DMSO) δ 10.16 (s, 1H), | 494 |
| | methoxy-4-((4- | 8.45 (d, J = 5.0 Hz, 1H), 8.08 (d, J = 7.4 Hz, | |
| | methylpyrimidin-2- | 1H), 7.60 (d, J = 8.9 Hz, 2H), 7.57-7.51 (m, | |
| | yl)oxy)phenyl)imidazo[1,2- | 2H), 7.30 (d, J = 8.1 Hz, 1H), 7.21 (d, J = 1.9 | |
| | a]pyrimidin-2- | Hz, 1H), 7.14 (d, J = 5.1 Hz, 1H), 7.02 (dd, J = | |
| | yl)phenyl)acrylamide | 8.1, 1.9 Hz, 1H), 6.89 (s, 2H), 6.43 (dd, J = 17.0, | |
| | | 10.1 Hz, 1H), 6.30 (d, J = 7.4 Hz, 1H), 6.24 (dd, | |
| | | J = 17.0, 2.0 Hz, 1H), 5.74 (dd, J = 10.1, 2.0 Hz, | |
| | | 1H), 3.65 (s, 3H), 2.44 (s, 3H). | |
| 7 | N-(4-(4-amino-5- | ¹H NMR (400 MHz, MeOD) δ 8.34 (d, J = 5.1 | 532 |
| | cyano-3-(3-methoxy-4- | Hz, 1H), 8.21 (s, 1H), 7.70 (d, J = 8.5 Hz, 2H), | |
| | ((4-methylpyrimidin-2- | 7.32 (d, J = 8.6 Hz, 2H), 7.11 (dd, J = 21.6, 6.6 | |
| | yl)oxy)phenyl)-1- | Hz, 2H), 6.95 (dd, J = 10.6, 2.6 Hz, 2H), 6.42 (d, | |
| | methyl-1H-pyrrolo[2,3- | J = 9.5 Hz, 1H), 6.39 (d, J = 2.4 Hz, 1H), 5.78 | |
| | b]pyridin-2- | (dd, J = 9.5, 2.4 Hz, 1H), 3.72 (s, 3H), 3.56 (s, | |
| | yl)phenyl)acrylamide | 3H), 2.46 (s, 3H). | |
| 8 | N-(4-(4-amino-7- | ¹H NMR (400 MHz, DMSO) δ 10.29 (s, 1H), | 532 |
| | cyano-3-(3-methoxy-4- | 8.40 (d, J = 5.2 Hz, 1H), 8.20 (s, 1H), 7.72 (m, | |
| | ((4-methylpyrimidin-2- | 2H), 7.35 (d, J = 8.4 Hz, 2H), 7.15 (d, J = 8.0 | |
| | yl)oxy)phenyl)-1- | Hz, 1H), 7.09 (d, J = 5.2 Hz, 1H), 6.99 (d, J = | |
| | methyl-1H-pyrrolo[3,2- | 1.6 Hz, 1H), 6.86 (m, 1H), 6.49-6.40 (m, 1H), | |
| | c]pyridin-2- | 6.29 (m, 1H), 5.78 (m, 1H), 3.81 (s, 3H), 3.55 (s, | |
| | yl)phenyl)acrylamide | 3H), 2.38 (s, 3H). | |
| 9 | N-(4-(4-amino-5-(3- | ¹H NMR (400 MHz, DMSO) δ 10.27 (s, 1H), | 522 |
| | methoxy-4-((4- | 8.40 (d, J = 5.0 Hz, 1H), 7.71 (d, J = 8.6 Hz, | |
| | methylpyrimidin-2- | 2H), 7.34 (d, J = 8.6 Hz, 2H), 7.10 (dd, J = 14.6, | |
| | yl)oxy)phenyl)-2,7- | 6.5 Hz, 2H), 6.94 (d, J = 1.7 Hz, 1H), 6.83 (dd, | |
| | dimethyl-7H- | J = 8.0, 1.8 Hz, 1H), 6.44 (dd, J = 17.0, 10.1 Hz, | |
| | pyrrolo[2,3- | 1H), 6.27 (dd, J = 17.0, 1.9 Hz, 1H), 5.91 (s, | |
| | d]pyrimidin-6- | 1H), 5.77 (dd, J = 10.1, 2.0 Hz, 1H), 3.56 (s, | |
| | yl)phenyl)acrylamide | 3H), 3.52 (s, 3H), 2.45 (s, 3H), 2.39 (s, 3H). | |
| 10 | N-(4-(4-amino-2- | ¹H NMR (400 MHz, DMSO) δ 10.28 (s, 1H), | 542 |
| | chloro-5-(3-methoxy-4- | 8.40 (d, J = 5.0 Hz, 1H), 7.72 (d, J = 8.3 Hz, | |
| | ((4-methylpyrimidin-2- | 2H), 7.35 (d, J = 8.4 Hz, 2H), 7.13 (d, J = 8.1 | |
| | yl)oxy)phenyl)-7- | Hz, 1H), 7.09 (d, J = 5.0 Hz, 1H), 6.95 (s, 1H), | |
| | methyl-7H-pyrrolo[2,3- | 6.84 (d, J = 7.9 Hz, 1H), 6.44 (dd, J = 17.0, 9.9 | |
| | d]pyrimidin-6- | Hz, 1H), 6.27 (d, J = 16.9 Hz, 1H), 5.78 (d, J = | |
| | yl)phenyl)acrylamide | 11.1 Hz, 1H), 3.54 (d, J = 11.8 Hz, 6H), 2.39 (s, | |
| | | 3H). | |
| 11 | N-(4-(4-methoxy-5-(3- | ¹H NMR (400 MHz, DMSO) δ 10.31 (s, 1H), | 523 |
| | methoxy-4-((4- | 8.51 (s, 1H), 8.40 (d, J = 4.6 Hz, 1H), 7.76 (d, | |
| | methylpyrimidin-2- | J = 8.0 Hz, 2H), 7.38 (d, J = 8.0 Hz, 2H), 7.03 (m, | |
| | yl)oxy)phenyl)-7- | 3H), 6.78 (d, J = 8.0 Hz, 1H), 6.45 (dd, J = 17.1, | |
| | methyl-7H-pyrrolo[2,3- | 10.1 Hz, 1H), 6.28 (d, J = 16.1 Hz, 1H), 5.78 (d, | |
| | d]pyrimidin-6- | J = 9.8 Hz, 1H), 4.00 (s, 3H), 3.66 (s, 3H), 3.51 | |
| | yl)phenyl)acrylamide | (s, 3H), 2.39 (s, 3H). | |
| 12 | N-(4-(4-cyclopropyl-5- | ¹H NMR (400 MHz, DMSO) δ 10.30 (s, 1H), | 533 |
| | (3-methoxy-4-((4- | 8.66 (s, 1H), 8.40 (d, J = 5.0 Hz, 1H), 7.74 (d, | |
| | methylpyrimidin-2- | J = 8.5 Hz, 2H), 7.41 (d, J = 8.5 Hz, 2H), 7.11- | |
| | yl)oxy)phenyl)-7- | 7.03 (m, 3H), 6.90 (dd, J = 8.1, 1.7 Hz, 1H), 6.44 | |
| | methyl-7H-pyrrolo[2,3- | (dd, J = 17.0, 10.1 Hz, 1H), 6.28 (dd, J = 17.0, | |
| | d]pyrimidin-6- | 1.8 Hz, 1H), 5.78 (dd, J = 10.2, 1.8 Hz, 1H), 3.68 | |
| | yl)phenyl)acrylamide | (s, 3H), 3.55 (s, 3H), 2.36 (s, 3H), 2.04 (dt, J = | |
| | | 12.8, 4.1 Hz, 1H), 1.18 (s, 2H), 0.92 (dd, J = 7.9, | |
| | | 3.1 Hz, 2H). | |
| 13 | N-(4-(4- | ¹H NMR (400 MHz, DMSO) δ 10.34 (s, 1H), | 559 |
| | (difluoromethoxy)-5-(3- | 8.61 (s, 1H), 8.40 (d, J = 4.8 Hz, 1H), 7.91 (m, | |
| | methoxy-4-((4- | 3H), 7.41 (d, J = 8.4 Hz, 2H), 7.09 (m, 2H), 7.00 | |
| | methylpyrimidin-2- | (d, J = 8.0 Hz, 1H), 6.79-6.71 (m, 1H), 6.45 (m, | |
| | yl)oxy)phenyl)-7- | 1H), 6.29 (m, 1H), 5.79 (m, 1H), 3.71 (s, 3H), | |
| | methyl-7H-pyrrolo[2,3- | 3.54 (s, 3H), 2.38 (s, 3H). | |
| | d]pyrimidin-6- | | |
| | yl)phenyl)acrylamide | | |
| 14 | N-(4-(4-cyano-5-(3- | ¹H NMR (400 MHz, DMSO) δ 10.37 (s, 1H), | 518 |
| | methoxy-4-((4- | 9.07 (s,1H), 8.40 (d, J = 5.0 Hz, 1H), 7.80 (d, J = | |
| | methylpyrimidin-2- | 8.7 Hz, 2H), 7.47 (d, J = 8.6 Hz, 2H), 7.21 (d, | |
| | yl)oxy)phenyl)-7- | J = 1.9 Hz, 1H), 7.12-7.07 (m, 2H), 6.83 (dd, J = | |
| | methyl-7H-pyrrolo[2,3- | 8.1, 1.9 Hz, 1H), 6.45 (dd, J = 16.9, 10.1 Hz, | |
| | d]pyrimidin-6- | 1H), 6.29 (dd, J = 17.0, 2.0 Hz, 1H), 5.80 (dd, | |
| | yl)phenyl)acrylamide | J = 10.1, 2.0 Hz, 1H), 3.78 (s, 3H), 3.61 (s, 3H), | |
| | | 2.38 (s, 3H). | |
| 15 | N-(4-(4-amino-6-(3- | ¹H NMR (400 MHz, DMSO) δ 10.23 (s, 1H), | 508 |
| | methoxy-4-((4- | 8.40 (d, J = 5.2 Hz, 1H), 8.20 (s, 1H), 7.69 (d, | |
| | methylpyrimidin-2- | J = 8.4 Hz, 2H), 7.26 (d, J = 8.4 Hz, 2H), 7.16 (d, | |
| | yl)oxy)phenyl)-7- | J = 8.0 Hz, 1H), 7.09 (m, 2H), 6.95 (m, 1H), | |
| | methyl-7H-pyrrolo[2,3- | 6.42 (m, 1H), 6.28 (m, 1H), 5.76 (m, 1H), 3.69 | |
| | d]pyrimidin-5- | (s, 3H), 3.55 (s, 3H), 2.40 (s, 3H). | |
| | yl)phenyl)acrylamide | | |
| 16 | N-(4-(4-amino-5-(4- | ¹H NMR (400 MHz, DMSO) δ 10.05 (s, 1H), | 486 |
| | (pyrrolidine-1-car- | 8.19 (s, 1H), 7.61 (m, 1H), 7.55 (d, J = 8.0 Hz, | |
| | bonyl)phenyl)pyrazolo[5,1- | 2H), 7.51 (m, 1H), 7.37 (t, J = 8.4 Hz, 1H), 7.33 | |
| | f][1,2,4]triazin- | (d, J = 8.0 Hz, 2H), 5.81 (s, 1H), 5.56 (s, 1H), | |
| | 6-yl)-3-fluoro- | 3.49-3.42 (m, 4H), 1.94 (s, 3H), 1.90-1.79 (m, | |
| | phenyl)methacrylamide | 4H). | |
| 17 | N-(4-(4-amino-5-(3- | ¹H NMR (400 MHz, MeOD) δ 7.98 (s, 1H), 7.86 | 510 |
| | methoxy-4-((5- | (s, 2H), 7.51 (d, J = 8.6 Hz, 2H), 7.41 (d, J = 8.6 | |
| | (methylamino)pyrimidin- | Hz, 2H), 7.18 (d, J = 8.0 Hz, 1H), 7.04 (s, 1H), | |
| | 2-yl)oxy)phe- | 6.96 (dd, J = 8.1, 1.4 Hz, 1H), 6.44-6.19 (m, | |
| | nyl)pyrazolo[5,1- | 2H), 5.67 (dd, J = 9.6, 1.9 Hz, 1H), 3.56 (s, 3H), | |
| | f][1,2,4]triazin-6- | 2.71 (s, 3H). | |
| | yl)phenyl)acrylamide | | |
| 18 | N-(4-(4-amino-5-(3- | ¹H NMR (400 MHz, DMSO) δ 10.21 (s, 1H), | 484 |
| | methoxy-4- | 8.16 (s, 1H), 7.62 (d, J = 8.8 Hz, 2H), 7.39 (d, | |
| | (pyrrolidine-1-car- | J = 8.8 Hz, 2H), 7.30 (d, J = 7.6 Hz, 1H), 7.15 (s, | |
| | bonyl)phenyl)pyrazolo[5,1- | 1H), 7.00 (d, J = 8.4 Hz, 1H), 6.42 (m, 1H), 6.25 | |
| | f][1,2,4]triazin- | (m, 1H), 5.76 (m, 1H), 3.73 (s, 3H), 3.46 (m, | |
| | 6-yl)phenyl)acrylamide | 2H), 3.21 (m, 2H), 1.93-1.74 (m, 4H). | |
| 19 | N-(4-(4-amino-5-(3- | ¹H NMR (400 MHz, DMSO) δ 10.22 (s, 1H), | 483 |
| | methoxy-4-((1-methyl- | 8.23 (d, J = 77.9 Hz, 2H), 7.80-7.55 (m, 3H), | |
| | 1H-pyrazol-3-yl)oxy)phe- | 7.42 (d, J = 8.4 Hz, 2H), 7.23-7.06 (m, 2H), | |
| | nyl)pyrazolo[5,1- | 6.91 (d, J = 8.2 Hz, 1H), 6.43 (dd, J = 16.9, 10.1 | |
| | f][1,2,4]triazin-6- | Hz, 1H), 6.26 (d, J = 16.9 Hz, 1H), 5.77 (dd, J = | |
| | yl)phenyl)acrylamide | 8.9, 6.2 Hz, 2H), 3.73 (d, J = 2.4 Hz, 6H). | |
| 20 | N-(4-(4-amino-5-(4- | ¹H NMR (400 MHz, MeOD) δ 8.56 (d J = 4.8 | 451 |
| | (pyrimidin-2- | Hz, 2H), 7.99 (s, 1H), 7.50 (d, J = 8.4 Hz, 2H), | |
| | yloxy)phenyl)pyrazolo[5,1- | 7.44 (d, J = 8.4 Hz, 2H), 7.38 (d, J = 8.4 Hz, | |
| | f][1,2,4]triazin-6- | 2H), 7.26 (d, J = 8.4 Hz, 2H), 7.19 (t, J = 4.8 Hz, | |
| | yl)phenyl)acrylamide | 1H), 6.30 (m, 2H), 5.68 (m, 1H). | |
| 21 | 2-(4- | ¹H NMR (400 MHz, MeOD) δ 8.34 (d, J = 4.8 | 550 |
| | acrylamidophenyl)-4- | Hz, 1H), 7.93 (s, 1H), 7.68 (d, J = 8.4 Hz, 2H), | |
| | amino-3-(3-methoxy-4- | 7.30 (d, J = 8.4 Hz, 2H), 7.14-7.06 (m, 2H), | |
| | ((4-methylpyrimidin-2- | 6.93 (d, J = 8.8 Hz, 2H), 6.46-6.33 (m, 2H), | |
| | yl)oxy)phenyl)-1- | 5.78 (m, 1H), 3.66 (s, 3H), 3.56 (s, 3H), 2.46 (s, | |
| | methyl-1H-pyrrolo[3,2- | 3H). | |
| | c]pyridine-7- | | |
| | carboxamide | | |
| 22 | N-(4-(4-amino-7- | ¹H NMR (400 MHz, MeOD) δ 8.39 (d, J = 4.8 | 518 |
| | cyano-3-(3-methoxy-4- | Hz, 1H), 8.05 (s, 1H), 7.62 (d, J = 8.8 Hz, 2H), | |
| | ((4-methylpyrimidin-2- | 7.39 (d, J = 8.8 Hz, 2H), 7.25 (d, J = 8.0 Hz, | |
| | yl)oxy)phenyl)-1H- | 1H), 7.15-7.07 (m, 2H), 7.07-6.98 (m, 1H), | |
| | pyrrolo[3,2-c]pyridin-2- | 6.49-6.31 (m, 2H), 5.77 (m, 1H), 3.63 (s, 3H), | |
| | yl)phenyl)acrylamide | 2.49 (s, 3H). | |
| 23 | 4-(2-(4- | ¹H NMR (400 MHz, DMSO) δ 12.45 (s, 1H), | 493 |
| | acrylamidophenyl)-4- | 10.22 (s, 1H), 8.20-8.09 (m, 2H), 7.68 (d, J = | |
| | amino-7-cyano-1H- | 7.8 Hz, 1H), 7.60 (d, J = 8.6 Hz, 2H), 7.33 (d, | |
| | pyrrolo[3,2-c]pyridin-3- | J = 8.6 Hz, 2H), 7.07 (s, 1H), 6.96 (d, J = 7.8 Hz, | |
| | yl)-N-cyclopropyl-2- | 1H), 6.46-6.37 (m, 1H), 6.25 (dd, J = 17.0, 1.6 | |
| | methoxybenzamide | Hz, 1H), 5.76 (dd, J = 10.1, 1.7 Hz, 3H), 3.76 (s, | |
| | | 3H), 2.84 (d, J = 3.9 Hz, 1H), 0.76-0.65 (m, | |
| | | 2H), 0.56 (dd, J = 6.5, 3.8 Hz, 2H). | |
| 24 | 4-(6-(4- | ¹H NMR (400 MHz, DMSO) δ 10.25 (s, 1H), | 538 |
| | acrylamidophenyl)-4- | 8.71 (d, J = 7.0 Hz, 1H), 8.43 (s, 1H), 8.18 (s, | |
| | aminopyrazolo[5,1- | 1H), 7.63 (d, J = 1.3 Hz, 3H), 7.40 (s, 2H), 7.35- | |
| | f][1,2,4]triazin-5-yl)-2- | 6.93 (m, 3H), 6.43 (m, 1H), 6.26 (m, 1H), 5.77 | |
| | (difluoromethoxy)-N- | (m, 2H), 5.39-5.13 (m, 1H), 4.65-4.37 (m, | |
| | ((1r,3r)-3- | 1H), 2.62-2.52 (m, 2H), 2.41 (m, 2H). | |
| | fluorocyclobutyl)benzamide | | |
| 25 | (S)-4-(6-(4- | ¹H NMR (400 MHz, DMSO) δ 10.24 (s, 1H), | 488 |
| | acrylamidophenyl)-4- | 8.58 (d, J = 6.5 Hz, 1H), 8.40 (s, 1H), 8.17 (s, | |
| | aminopyrazolo[5,1- | 1H), 7.66 (dd, J = 18.2, 8.2 Hz, 3H), 7.36 (d, J = | |
| | f][1,2,4]triazin-5-yl)-2- | 8.7 Hz, 3H), 7.28 (d, J = 7.8 Hz, 1H), 6.43 (dd, | |
| | fluoro-N- | J = 17.0, 10.0 Hz, 1H), 6.26 (dd, J = 16.9, 1.8 Hz, | |
| | (tetrahydrofuran-3- | 1H), 5.74 (dt, J = 42.5, 21.2 Hz, 2H), 4.45 (s, | |
| | yl)benzamide | 1H), 3.85 (dt, J = 14.4, 6.8 Hz, 2H), 3.72 (td, J = | |
| | | 8.2, 5.7 Hz, 1H), 3.60 (dd, J = 9.0, 4.0 Hz, 1H), | |
| | | 2.17 (dd, J = 12.7, 7.6 Hz, 1H), 1.91 (dd, J = | |
| | | 12.4, 4.8 Hz, 1H). | |
| 26 | 4-(6-(4- | ¹H NMR (400 MHz, DMSO) δ 10.23 (s, 1H), | 484 |
| | acrylamidophenyl)-4- | 8.34 (d, J = 7.7 Hz, 2H), 8.16 (s, 1H), 7.74 (d, | |
| | aminopyrazolo[5,1- | J = 7.8 Hz, 1H), 7.62 (d, J = 8.7 Hz, 2H), 7.41 (d, | |
| | f][1,2,4]triazin-5-yl)-N- | J = 8.7 Hz, 2H), 7.20 (s, 1H), 7.02 (dd, J = 7.8, | |
| | cyclobutyl-2- | 1.2 Hz, 1H), 6.43 (dd, J = 17.0, 10.1 Hz, 1H), | |
| | methoxybenzamide | 6.25 (dd, J = 17.0, 1.9 Hz, 1H), 5.76 (dd, J = | |
| | | 10.1, 1.9 Hz, 1H), 5.61 (s, 1H), 4.43 (dd, J = | |
| | | 16.3, 8.3 Hz, 1H), 3.82 (s, 3H), 2.24 (dt, J = | |
| | | 11.2, 5.3 Hz, 2H), 2.03 (dt, J = 11.6, 9.6 Hz, | |
| | | 2H), 1.76-1.59 (m, 2H). | |
| 27 | 4-(4-amino-6-(4- | ¹H NMR (400 MHz, DMSO) δ 9.85 (s, 1H), 8.58 | 470 |
| | methacrylamidophenyl) | (s, 1H), 8.40 (s, 1H), 8.16 (s, 1H), 7.96 (d, J = | |
| | pyrazolo[5,1- | 7.7 Hz, 2H), 7.62 (d, J = 8.2 Hz, 2H), 7.49 (d, | |
| | f][1,2,4]triazin-5-yl)-N- | J = 7.7 Hz, 2H), 7.33 (d, J = 8.2 Hz, 2H), 5.78 (s, | |
| | isobutylbenzamide | 1H), 5.51 (s, 2H), 3.11 (t, J = 5.9 Hz, 2H), 1.93 | |
| | | (s, 3H), 1.90-1.81 (m, 1H), 0.91 (d, J = 6.4 Hz, | |
| | | 6H). | |
| 29 | N-(4-(4-amino-5-(3- | ¹H NMR (400 MHz, DMSO) δ 10.27 (s, 1H), | 481 |
| | methoxy-4-(pyrimidin- | 8.66 (d, J = 4.7 Hz, 2H), 8.45 (s, 1H), 8.16 (s, | |
| | 2-yloxy)phenyl) | 1H), 7.66 (d, J = 8.6 Hz, 2H), 7.47 (d, J = 8.6 | |
| | pyrazolo[5,1- | Hz, 2H), 7.33 (d, J = 8.0 Hz, 1H), 7.27 (t, J = 4.7 | |
| | f][1,2,4]triazin-6- | Hz, 1H), 7.23 (s, 1H), 7.05 (d, J = 8.0 Hz, 1H), | |
| | yl)phenyl)acrylamide | 6.44 (m, 1H), 6.31-6.22 (m, 1H), 5.81-5.72 | |
| | | (m, 1H), 5.54 (s, 1H), 3.63 (s, 3H). | |
| 30 | N-(4-(4-amino-5-(3- | ¹H NMR (400 MHz, DMSO) δ 10.26 (s, 1H), | 469 |
| | fluoro-4-(pyrimidin-2- | 8.72 (d, J = 4.8 Hz, 2H), 8.17 (s, 1H), 7.66 (d, | |
| | yloxy)phenyl)pyrazolo[5,1- | J = 8.6 Hz, 2H), 7.57-7.45 (m, 2H), 7.44-7.34 | |
| | f][1,2,4]triazin-6- | (m, 3H), 7.30 (d, J = 8.3 Hz, 1H), 6.42 (d, J = | |
| | yl)phenyl)acrylamide | 10.1 Hz, 1H), 6.32-6.20 (m, 1H), 5.77 (m, 1H). | |
| 31 | N-(4-(4-amino-5-(4-((4- | ¹H NMR (400 MHz, DMSO) δ 10.24 (s, 1H), | 465 |
| | methylpyrimidin-2-yl)oxy)phe- | 8.51 (d, J = 5.0 Hz, 1H), 8.16 (s, 1H), 7.64 (d, | |
| | nyl)pyrazolo[5,1- | J = 8.7 Hz, 2H), 7.49 (d, J = 8.5 Hz, 2H), 7.42 (d, | |
| | f][1,2,4]triazin-6- | J = 8.7 Hz, 2H), 7.34 (d, J = 8.5 Hz, 2H), 7.19 | |
| | yl)phenyl)acrylamide | (d, J = 5.0 Hz, 1H), 6.43 (m, 1H), 6.26 (m, 1H), | |
| | | 5.76 (m, 1H), 2.44 (s, 3H). | |
| 32 | N-(4-(4-amino-5-(4- | ¹H NMR (400 MHz, DMSO) δ 10.25 (s, 1H), | 449 |
| | phenoxyphenyl)pyrazolo[5,1- | 8.15 (s, 1H), 7.61 (m, 2H), 7.49-7.34 (m, 6H), | |
| | f][1,2,4]triazin-6- | 7.22-7.09 (m, 5H), 6.44 (m, 1H), 6.27 (m, 1H), | |
| | yl)phenyl)acrylamide | 5.77 (m, 1H). | |
| 33 | N-(4-(4-amino-5-(4- | ¹H NMR (400 MHz, DMSO) δ 10.24 (s, 1H), | 450 |
| | (pyridin-2- | 8.41 (s, 1H), 8.25 (dd, J = 4.9, 1.5 Hz, 1H), 8.16 | |
| | yloxy)phenyl)pyrazolo[5,1- | (s, 1H), 7.95-7.87 (m, 1H), 7.64 (d, J = 8.7 Hz, | |
| | f][1,2,4]triazin-6- | 2H), 7.44 (dd, J = 19.2, 8.6 Hz, 4H), 7.27 (d, J = | |
| | yl)phenyl)acrylamide | 8.5 Hz, 2H), 7.20 (dd, J = 6.8, 5.2 Hz, 1H), 7.13 | |
| | | (d, J = 8.3 Hz, 1H), 6.43 (dd, J = 16.9, 10.1 Hz, | |
| | | 1H), 6.26 (dd, J = 17.0, 1.8 Hz, 1H), 5.77 (dd, | |
| | | J = 10.1, 1.8 Hz, 1H), 5.47 (s, 1H). | |
| 34 | N-(4-(4-amino-5-(4- | ¹H NMR (400 MHz, DMSO) δ 10.21 (s, 1H), | 441 |
| | (cyclopentyloxy)phe- | 8.32 (s, 1H), 8.12 (s, 1H), 7.60 (d, J = 8.6 Hz, | |
| | nyl)pyrazolo[5,1- | 2H), 7.40 (d, J = 8.6 Hz, 2H), 7.31 (d, J = 8.5 | |
| | f][1,2,4]triazin-6- | Hz, 2H), 7.03 (d, J = 8.6 Hz, 2H), 6.42 (dd, J = | |
| | yl)phenyl)acrylamide | 17.0, 10.1 Hz, 1H), 6.25 (dd, J = 17.0, 1.8 Hz, | |
| | | 1H), 5.76 (dd, J = 10.1, 1.9 Hz, 1H), 5.33 (d, J = | |
| | | 5.0 Hz, 1H), 4.89 (dd, J = 7.2, 4.6 Hz, 1H), 1.96 | |
| | | (dd, J = 13.0, 7.0 Hz, 2H), 1.74 (dd, J = 17.0, 9.2 | |
| | | Hz, 4H), 1.61 (dd, J = 7.3, 5.4 Hz, 2H). | |
| 35 | N-(4-(4-amino-5-(4- | ¹H NMR (400 MHz, DMSO) δ 10.03 (s, 1H), | 469 |
| | (pyrimidin-2-yloxy) | 8.70 (d, J = 4.8 Hz, 2H), 8.46 (s, 1H), 8.18 (d, | |
| | phenyl)pyrazolo[5,1- | J = 6.8 Hz, 1H), 8.02 (t, J = 8.3 Hz, 1H), 7.53 (d, | |
| | f][1,2,4]triazin-6-yl)-2- | J = 8.5 Hz, 2H), 7.40 (d, J = 8.5 Hz, 2H), 7.30 (m, | |
| | fluorophenyl)acrylamide | 3H), 6.62 (dd, J = 17.0, 10.2 Hz, 1H), 6.28 (d, | |
| | | J = 17.0 Hz, 1H), 5.78 (dd, J = 10.2, 1.7 Hz, 1H), | |
| | | 5.45 (s, 1H). | |
| 36 | N-(4-(4-amino-5-(4- | ¹H NMR (400 MHz, DMSO) δ 10.47 (s, 1H), | 469 |
| | (pyrimidin-2-yloxy) | 8.68 (d, J = 4.8 Hz, 2H), 8.49 (s, 1H), 8.20 (s, | |
| | phenyl)pyrazolo[5,1- | 1H), 7.73-7.64 (m, 1H), 7.47-7.34 (m, 4H), | |
| | f][1,2,4]triazin-6-yl)-3- | 7.29 (dd, J = 9.1, 5.4 Hz, 3H), 6.42 (dd, J = 17.0, | |
| | fluorophenyl)acrylamide | 10.0 Hz, 1H), 6.29 (dd, J = 17.0, 1.9 Hz, 1H), | |
| | | 5.81 (dd, J = 10.0, 1.9 Hz, 1H), 5.68 (s, 1H). | |
| 37 | 4-(2-(4- | ¹H NMR (400 MHz, DMSO) δ 10.26 (s, 1H), | 551 |
| | acrylamidophenyl)-4- | 8.24 (d, J = 7.8 Hz, 1H), 8.19 (s, 1H), 7.68 (d, | |
| | amino-7-cyano-1-(2- | J = 8.6 Hz, 2H), 7.57 (d, J = 7.8 Hz, 1H), 7.34 (d, | |
| | hydroxyethyl)-1H- | J = 8.5 Hz, 2H), 6.95-6.83 (m, 2H), 6.43 (dd, J = | |
| | pyrrolo[3,2-c]pyridin-3- | 17.0, 10.0 Hz, 1H), 6.27 (dd, J = 17.0, 2.0 Hz, | |
| | yl)-N-cyclobutyl-2- | 1H), 5.77 (dd, J = 10.1, 1.9 Hz, 1H), 4.90 (t, J = | |
| | methoxybenzamide | 5.2 Hz, 1H), 4.44-4.24 (m, 3H), 3.71 (s, 3H), | |
| | | 3.64-3.51 (m, 2H), 2.24-2.15 (m, 2H), 2.04-1.91 | |
| | | (m, 2H), 1.74-1.56 (m, 2H). | |
| 38 | 4-(6-(4- | ¹H NMR (400 MHz, DMSO) δ 10.22 (s, 1H), | 456 |
| | acrylamidophenyl)-4- | 8.59 (s, 1H), 8.35 (s, 1H), 8.16 (s, 1H), 7.96 (d, | |
| | aminopyrazolo[5,1-f] | J = 7.9 Hz, 2H), 7.61 (d, J = 8.3 Hz, 2H), 7.50 (d, | |
| | [1,2,4]triazin-5-yl)-N- | J = 7.8 Hz, 2H), 7.35 (d, J = 8.4 Hz, 2H), 6.42 | |
| | isobutylbenzamide | (dd, J = 16.9, 10.0 Hz, 1H), 6.25 (d, J = 17.1 Hz, | |
| | | 1H), 5.76 (d, J = 10.3 Hz, 1H), 5.48 (s, 1H), 3.11 | |
| | | (m, 2H), 1.92-1.80 (m, 1H), 0.91 (d, J = 6.5 Hz, | |
| | | 6H). | |
| 39 | 4-(6-(4- | ¹H NMR (400 MHz, DMSO) δ 10.23 (s, 1H), | 454 |
| | acrylamidophenyl)-4- | 8.74 (d, J = 7.5 Hz, 1H), 8.36 (s, 1H), 8.16 (s, | |
| | aminopyrazolo[5,1-f] | 1H), 7.96 (d, J = 8.2 Hz, 2H), 7.60 (d, J = 8.7 | |
| | [1,2,4]triazin-5-yl)-N- | Hz, 2H), 7.50 (d, J = 8.2 Hz, 2H), 7.35 (d, J = | |
| | cyclobutylbenzamide | 8.7 Hz, 2H), 6.42 (dd, J = 17.0, 10.1 Hz, 1H), | |
| | | 6.25 (dd, J = 17.0, 1.9 Hz, 1H), 5.76 (dd, J = | |
| | | 10.1, 1.9 Hz, 1H), 5.47 (s, 1H), 4.45 (dd, J = | |
| | | 16.3, 8.3 Hz, 1H), 2.22 (dd, m, 2H), 2.15-2.04 | |
| | | (m, 2H), 1.73-1.63 (m, 2H). | |
| 40 | 4-(6-(4- | ¹H NMR (400 MHz, DMSO) δ 10.24 (s, 1H), | 472 |
| | acrylamidophenyl)-4- | 8.59 (d, J = 7.9 Hz, 1H), 8.17 (s, 1H), 7.69-7.62 | |
| | aminopyrazolo[5,1-f] | (m, 3H), 7.50-7.32 (m, 3H), 7.27 (d, J = 7.9 Hz, | |
| | [1,2,4]triazin-5-yl)-N- | 1H), 6.43 (m, 1H), 6.26 (d, J = 17.0 Hz, 1H), | |
| | cyclobutyl-2- | 5.77 (d, J = 9.9 Hz, 1H), 4.62-4.15 (m, 1H), | |
| | fluorobenzamide | 2.23 (s, 2H), 2.10-1.92 (m, 2H), 1.68 (m, 2H). | |
| 41 | methyl 4-(6-(4- | ¹H NMR (400 MHz, DMSO) δ 10.23 (s, 1H), | 445 |
| | acrylamidophenyl)-4- | 8.35 (s, 1H), 8.16 (s, 1H), 7.74 (d, J = 7.8 Hz, | |
| | aminopyrazolo[5,1-f] | 1H), 7.62 (d, J = 8.7 Hz, 2H), 7.37 (d, J = 8.7 | |
| | [1,2,4]triazin-5-yl)-2- | Hz, 2H), 7.22 (s, 1H), 6.99 (dd, J = 7.8, 1.3 Hz, | |
| | methoxybenzoate | 1H), 6.43 (dd, J = 16.9, 10.1 Hz, 1H), 6.25 (dd, | |
| | | J = 17.0, 1.9 Hz, 1H), 5.99-5.52 (m, 2H), 3.81 (s, | |
| | | 3H), 3.76 (s, 3H). | |
| 42 | 4-(6-(4-acrylamidophenyl)-4- | ¹H NMR (400 MHz, DMSO) δ 10.23 (s, 1H), | 512 |
| | aminopyrazolo[5,1-f] | 8.74 (t, J = 6.4 Hz, 1H), 8.34 (s, 1H), 8.16 (s, | |
| | [1,2,4]triazin-5-yl)-2- | 1H), 7.84 (d, J = 7.9 Hz, 1H), 7.63 (d, J = 8.7 | |
| | methoxy-N-(2,2,2- | Hz, 2H), 7.40 (d, J = 8.7 Hz, 2H), 7.24 (s, 1H), | |
| | trifluoroethyl)benzamide | 7.04 (d, J = 7.8 Hz, 1H), 6.43 (dd, J = 16.9, 10.1 | |
| | | Hz, 1H), 6.25 (dd, J = 17.0, 1.9 Hz, 1H), 5.75 | |
| | | (m, 2H), 4.15 (dd, J = 9.7, 6.6 Hz, 2H), 3.84 (s, 3H). | |
| 43 | 4-(6-(4- | ¹H NMR (400 MHz, DMSO) δ 10.23 (s, 1H), | 486 |
| | acrylamidophenyl)-4- | 8.82 (d, J = 6.3 Hz, 1H), 8.17 (s, 1H), 7.75 (d, | |
| | aminopyrazolo[5,1-f] | J = 7.8 Hz, 1H), 7.63 (d, J = 8.7 Hz, 2H), 7.41 (d, | |
| | [1,2,4]triazin-5-yl)-2- | J = 8.7 Hz, 2H), 7.22 (s, 1H), 7.13-6.92 (m, | |
| | methoxy-N-(oxetan-3- | 1H), 6.43 (m, 1H), 6.25 (m, 1H), 5.76 (m, 1H), | |
| | yl)benzamide | 5.01 (d, J = 6.7 Hz, 1H), 4.78 (t, J = 7.0 Hz, 2H), | |
| | | 4.59 (t, J = 6.5 Hz, 2H), 3.83 (s, 3H). | |
| 44 | 4-(6-(4- | ¹H NMR (400 MHz, DMSO) δ 10.22 (s, 1H), | 524 |
| | acrylamidophenyl)-4- | 8.64 (t, J = 5.6 Hz, 1H), 8.16 (s, 1H), 7.88 (d, J = | |
| | aminopyrazolo[5,1-f] | 7.6 Hz, 1H), 7.62 (m, 3H), 7.40 (d, J = 8.8 Hz, | |
| | [1,2,4]triazin-5-yl)-2- | 2H), 7.22 (s, 1H), 7.04 (m, 1H), 6.43 (m, 1H), | |
| | methoxy-N-((1-methyl- | 6.30-6.12 (m, 2H), 5.76 (m, 1H), 4.46 (d, J = | |
| | 1H-pyrazol-3-yl) | 5.6 Hz, 2H), 3.84 (s, 3H), 3.80 (s, 3H). | |
| | methyl)benzamide | | |
| 45 | 4-(6-(4- | ¹H NMR (400 MHz, DMSO) δ 10.23 (s, 1H), | 498 |
| | acrylamidophenyl)-4- | 8.18 (d, J = 14.2 Hz, 2H), 7.79 (d, J = 7.8 Hz, | |
| | aminopyrazolo[5,1-f] | 1H), 7.62 (d, J = 8.7 Hz, 2H), 7.40 (d, J = 8.7 | |
| | [1,2,4]triazin-5-yl)-N- | Hz, 2H), 7.20 (s, 1H), 7.03 (d, J = 7.8 Hz, 1H), | |
| | (cyclobutylmethyl)-2- | 6.43 (dd, J = 17.0, 10.1 Hz, 1H), 6.25 (dd, J = | |
| | methoxybenzamide | 17.0, 1.9 Hz, 1H), 5.76 (dd, J = 10.2, 1.9 Hz, | |
| | | 1H), 3.82 (s, 3H), 3.34 (d, J = 9.7 Hz, 2H), 2.57- | |
| | | 2.53 (m, 1H), 2.02-1.97 (m, 2H), 1.87-1.83 (m, | |
| | | 2H), 1.77-1.73(m, 2H) | |
| 46 | 4-(6-(4- | ¹H NMR (400 MHz, DMSO) δ 10.23 (s, 1H), | 500 |
| | acrylamidophenyl)-4- | 8.26 (d, J = 7.5 Hz, 1H), 8.16 (s, 1H), 7.74 (d, | |
| | aminopyrazolo[5,1-f] | J = 7.8 Hz, 1H), 7.62 (d, J = 8.7 Hz, 2H), 7.41 (d, | |
| | [1,2,4]triazin-5-yl)-N- | J = 8.7 Hz, 2H), 7.20 (s, 1H), 7.02 (d, J = 7.9 Hz, | |
| | ((1s,3s)-3- | 1H), 6.43 (dd, J = 17.0, 10.1 Hz, 1H), 6.25 (dd, | |
| | hydroxycyclobutyl)-2- | J = 16.9, 1.9 Hz, 1H), 5.76 (dd, J = 10.1, 1.9 Hz, | |
| | methoxybenzamide | 1H), 5.10 (d, J = 6.0 Hz, 1H), 3.98-3.80 (m, | |
| | | 2H), 3.33 (s, 3H), 2.82-2.38 (m, 2H), 1.86 (m, 2H). | |
| 47 | 4-(6-(4- | ¹H NMR (400 MHz, DMSO) δ 10.23 (s, 1H), | 509 |
| | acrylamidophenyl)-4- | 8.59 (t, J = 6.1 Hz, 1H), 8.35 (s, 1H), 8.16 (s, | |
| | aminopyrazolo[5,1-f] | 1H), 7.82 (d, J = 7.8 Hz, 1H), 7.63 (d, J = 8.7 | |
| | [1,2,4]triazin-5-yl)-N- | Hz, 2H), 7.41 (d, J = 8.7 Hz, 2H), 7.23 (d, J = | |
| | ((1-cyanocyclopropyl) | 1.1 Hz, 1H), 7.05 (dd, J = 7.8, 1.3 Hz, 1H), 6.43 | |
| | methyl)-2- | (dd, J = 17.0, 10.1 Hz, 1H), 6.25 (dd, J = 17.0, | |
| | methoxybenzamide | 1.9 Hz, 1H), 5.76 (dd, J = 10.1, 1.9 Hz, 1H), 5.67 | |
| | | (s, 1H), 3.85 (s, 3H), 3.50 (d, J = 6.1 Hz, 2H), | |
| | | 1.28-1.11 (m, 4H). | |
| 48 | 4-(4-amino-6-(4-(2- | ¹H NMR (400 MHz, DMSO) δ 10.36 (s, 1H), | 474 |
| | fluoroacrylamido)phe- | 8.59 (t, J = 5.7 Hz, 1H), 8.36 (s, 1H), 8.17 (s, | |
| | nyl)pyrazolo[5,1-f] | 1H), 7.96 (d, J = 8.2 Hz, 2H), 7.67 (d, J = 8.7 | |
| | [1,2,4]triazin-5-yl)-N- | Hz, 2H), 7.50 (d, J = 8.2 Hz, 2H), 7.37 (d, J = | |
| | isobutylbenzamide | 8.7 Hz, 2H), 5.82-5.36 (m, 3H), 3.11 (t, J = 6.4 | |
| | | Hz, 2H), 1.87 (m, 1H), 0.91 (d, J = 6.7 Hz, 6H). | |
| 49 | 4-(6-(4- | ¹H NMR (400 MHz, DMSO) δ 10.20 (s, 1H), | 498 |
| | acrylamidophenyl)-4- | 8.35 (s, 1H), 8.15 (d, J = 6.3 Hz, 2H), 7.80 (d, | |
| | aminopyrazolo[5,1-f] | J = 7.8 Hz, 1H), 7.62 (d, J = 8.7 Hz, 2H), 7.41 (d, | |
| | [1,2,4]triazin-5-yl)-N- | J = 8.7 Hz, 2H), 7.20 (d, J = 1.1 Hz, 1H), 7.03 | |
| | cyclobutyl-2-methoxy- | (dd, J = 7.8, 1.3 Hz, 1H), 6.43 (dd, J = 17.0, 10.1 | |
| | N-methylbenzamide | Hz, 1H), 6.25 (dd, J = 17.0, 2.0 Hz, 1H), 5.76 | |
| | | (dd, J = 10.1, 2.0 Hz, 1H), 5.57 (s, 1H), 3.84 (s, | |
| | | 3H), 2.34 (dd, J = 15.3, 5.7 Hz, 2H), 2.07-1.97 | |
| | | (m, 2H), 1.84 (dd, J = 15.3, 8.6 Hz, 2H), 1.50 (s, 3H). | |
| 50 | 4-(6-(4- | ¹H NMR (400 MHz, DMSO) δ 10.28 (s, 1H), | 458 |
| | acrylamidophenyl)-4- | 8.30 (d, J = 48.8 Hz, 2H), 8.03 (s, 1H), 7.79- | |
| | aminopyrazolo[5,1-f] | 7.64 (m, 4H), 6.47 (dd, J = 17.0, 10.1 Hz, 1H), | |
| | [1,2,4]triazin-5-yl)-N- | 6.28 (dd, J = 17.0, 1.9 Hz, 1H), 5.96 (s, 1H), | |
| | cyclobutylcyclohex-3- | 5.78 (dd, J = 10.1, 1.9 Hz, 1H), 4.21 (dd, J = | |
| | ene-1-carboxamide | 16.0, 8.0 Hz, 1H), 2.34 (s, 1H), 2.15 (d, J = 3.7 | |
| | | Hz, 2H), 2.14 (s, 4H), 2.15-1.72 (m, 4H), 1.60 | |
| | | (m, 2H). | |
| 51 | 4-(6-(4-acrylamido-3- | ¹H NMR (400 MHz, DMSO) δ 10.02 (s, 1H), | 474 |
| | fluorophenyl)-4- | 8.61 (t, J = 5.8 Hz, 1H), 8.43 (s, 1H), 8.19 (s, | |
| | aminopyrazolo[5,1-f] | 1H), 7.98 (t, J = 7.5 Hz, 3H), 7.53 (d, J = 8.2 Hz, | |
| | [1,2,4]triazin-5-yl)-N- | 2H), 7.21 (dd, J = 15.5, 4.8 Hz, 2H), 6.61 (dd, | |
| | isobutylbenzamide | J = 17.0, 10.2 Hz, 1H), 6.26 (dd, J = 17.0, 1.9 Hz, | |
| | | 1H), 5.78 (dd, J = 10.2, 1.9 Hz, 1H), 5.56 (s, | |
| | | 1H), 3.12 (t, J = 6.4 Hz, 2H), 1.87 (m, 1H), 0.91 | |
| | | (d, J = 6.7 Hz, 6H). | |
| 52 | 4-(6-(4-acrylamido-2- | ¹H NMR (400 MHz, DMSO) δ 10.44 (s, 1H), | 474 |
| | fluorophenyl)-4- | 8.61-8.28 (m, 2H), 8.20 (s, 1H), 7.87 (d, J = 8.2 | |
| | aminopyrazolo[5,1-f] | Hz, 2H), 7.63 (d, J = 12.1 Hz, 1H), 7.39 (dd, J = | |
| | [1,2,4]triazin-5-yl)-N- | 13.6, 7.3 Hz, 4H), 6.42 (dd, J = 16.9, 10.0 Hz, | |
| | isobutylbenzamide | 1H), 6.28 (dd, J = 17.0, 1.8 Hz, 1H), 6.01-5.65 | |
| | | (m, 2H), 3.08 (t, J = 6.4 Hz, 2H), 1.84 (m, 1H), | |
| | | 0.89 (d, J = 6.7 Hz, 6H). | |
| 53 | 4-(6-(4-acrylamido-3- | ¹H NMR (400 MHz, DMSO) δ 9.42 (s, 1H), 8.59 | 486 |
| | methoxyphenyl)-4- | (t, J = 5.7 Hz, 1H), 8.39 (s, 1H), 8.17 (s, 1H), | |
| | aminopyrazolo[5,1-f] | 8.06-7.92 (m, 3H), 7.53 (d, J = 8.1 Hz, 2H), | |
| | [1,2,4]triazin-5-yl)-N- | 7.00 (dd, J = 19.1, 4.9 Hz, 2H), 6.69 (dd, J = | |
| | isobutylbenzamide | 16.9, 10.1 Hz, 1H), 6.22 (dd, J = 16.9, 1.8 Hz, | |
| | | 1H), 5.71 (dd, J = 10.2, 1.8 Hz, 1H), 5.53 (s, | |
| | | 1H), 3.63 (d, J = 7.5 Hz, 3H), 3.12 (t, J = 6.4 Hz, | |
| | | 2H), 1.87 (m, 1H), 0.91 (d, J = 6.7 Hz, 6H). | |
| 54 | 4-(6-(4-acrylamido-2- | ¹H NMR (400 MHz, DMSO) δ 10.26 (s, 1H), | 486 |
| | methoxyphenyl)-4- | 8.47 (t, J = 5.8 Hz, 1H), 8.16 (s, 1H), 7.85 (d, J = | |
| | aminopyrazolo[5,1-f] | 8.1 Hz, 2H), 7.39 (s, 1H), 7.33-7.22 (m, 4H), | |
| | [1,2,4]triazin-5-yl)-N- | 6.43 (dd, J = 16.9, 10.1 Hz, 1H), 6.26 (dd, J = | |
| | isobutylbenzamide | 17.0, 1.8 Hz, 1H), 6.03-5.47 (m, 2H), 3.26 (s, | |
| | | 3H), 3.07 (t, J = 6.4 Hz, 2H), 1.82 (dd, J = 13.6, | |
| | | 6.8 Hz, 1H), 0.88 (d, J = 6.7 Hz, 6H). | |
| 55 | 4-(6-(4- | ¹H NMR (400 MHz, DMSO) δ 10.22 (s, 1H), | 498 |
| | acrylamidophenyl)-4- | 8.16 (s, 1H), 8.08 (d, J = 7.3 Hz, 1H), 7.76 (d, | |
| | aminopyrazolo[5,1-f] | J = 7.8 Hz, 1H), 7.62 (d, J = 8.7 Hz, 2H), 7.41 (d, | |
| | [1,2,4]triazin-5-yl)-N- | J = 8.7 Hz, 2H), 7.20 (s, 1H), 7.02 (d, J = 7.8 Hz, | |
| | cyclopentyl-2- | 1H), 6.43 (dd, J = 16.9, 10.1 Hz, 1H), 6.25 (dd, | |
| | methoxybenzamide | J = 17.0, 2.0 Hz, 1H), 5.76 (dd, J = 10.1, 1.9 Hz, | |
| | | 1H), 4.24 (dd, J = 13.5, 6.3 Hz, 1H), 3.82 (s, | |
| | | 3H), 1.97-1.86 (m, 2H), 1.69 (m, 2H), 1.63-1.46 | |
| | | (m, 4H). | |
| 56 | 4-(6-(4- | ¹H NMR (400 Hz, DMSO) δ 10.22 (s, 1H), 8.19- | 516 |
| | acrylamidophenyl)-4- | 8.16 (m, 2H), 7.88(d, J = 8.0 Hz, 1H), 7.62 (d, | |
| | aminopyrazolo[5,1-f] | J = 8.8 Hz, 2H), 7.40 (d, J = 8.8 Hz, 2H), 7.24 (s, | |
| | [1,2,4]triazin-5-yl)-2- | 1H), 7.04 (d, J = 9.2 Hz, 1H), 6.46-6.39 (m, 1H), | |
| | methoxy-N-(2-methoxy- | 6.24 (dd, J = 1.6, 2.0 Hz, 1H), 5.76 (dd, J = 10.1, | |
| | 2-methylpropyl) | 1.9 Hz, 1H), 3.85(s, 3H), 3.37 (d, J = 5.6 Hz, | |
| | benzamide | 2H), 3.17 (s, 3H), 1.16 (s, 6H). | |
| 57 | 4-(6-(4- | ¹H NMR (400 MHz, DMSO) δ 10.22 (s, 1H), | 502 |
| | acrylamidophenyl)-4- | 8.23 (t, J = 7.2 Hz, 1H), 8.16 (s, 1H), 7.89 (d, J = | |
| | aminopyrazolo[5,1-f] | 7.9 Hz, 1H), 7.62 (d, J = 8.7 Hz, 2H), 7.40 (d, | |
| | [1,2,4]triazin-5-yl)-N- | J = 8.7 Hz, 2H), 7.24 (s, 1H), 7.07-7.01 (m, 1H), | |
| | (2-hydroxy-2- | 6.43 (dd, J = 16.9, 10.1 Hz, 1H), 6.25 (dd, J = | |
| | methylpropyl)-2- | 17.0, 1.9 Hz, 1H), 5.76 (dd, J = 10.1, 1.9 Hz, | |
| | methoxybenzamide | 1H), 4.65 (s, 1H), 3.85 (s, 3H), 3.29 (d, J = 5.7 | |
| | | Hz, 2H), 1.15 (s, 6H). | |
| 58 | 4-(6-(4- | ¹H NMR (400 Hz, DMSO) δ 10.22 (s, 1H), 8.15 | 512 |
| | acrylamidophenyl)-4- | (s, 1H), 8.02 (d, J = 8.0 Hz, 1H), 7.79 (d, J = 3.8 | |
| | aminopyrazolo[5,1-f] | Hz, 1H), 7.62 (d, J = 4.2 Hz, 2H), 7.40 (d, J = | |
| | [1,2,4]triazin-5-yl)-N- | 8.0 Hz, 2H), 7.20 (s, 1H), 7.02 (d, J = 8.0 Hz, | |
| | cyclohexyl-2- | 1H), 6.46-6.39 (m, 1H), 6.25 (d, J = 2.0, Hz, | |
| | methoxybenzamide | 1H), 5.76 (dd, J = 2.0, 1.6 Hz, 1H), 3.82 (s, 4H), | |
| | | 1.86-1.57 (m, 5H), 1.37-1.21(m, 5H). | |
| 59 | 4-(2-(4- | ¹H NMR (400 MHz, DMSO) δ 10.28 (s, 1H), | 565 |
| | acrylamidophenyl)-4- | 8.24 (d, J = 8.0 Hz, 2H), 8.21 (s, 1H), 7.69 (d, | |
| | amino-7-cyano-1-(2- | J = 8.4 Hz, 2H), 7.56 (d, J = 8.0 Hz, 1H), 7.33 (d, | |
| | methoxyethyl)-1H- | J = 8.4 Hz, 2H), 6.94 (d, J = 1.2 Hz, 2H), 6.87 | |
| | pyrrolo[3,2-c]pyridin-3- | (m, 1H), 6.43 (m, 1H), 6.27 (m, 1H), 5.78 (m, | |
| | yl)-N-cyclobutyl-2- | 1H), 4.38 (m, 3H), 3.72 (s, 3H), 3.51 (t, J = 5.6 | |
| | methoxybenzamide | Hz, 2H), 3.07 (s, 3H), 2.19 (m, 2H), 2.04-1.92 | |
| | | (m, 2H), 1.69-1.60 (m, 2H). | |
| 60 | 4-(6-(4- | ¹H NMR (400 MHz, DMSO) δ 10.22 (s, 1H), | 458 |
| | acrylamidophenyl)-4- | 8.24 (d, J = 5.8 Hz, 1H), 8.16 (s, 1H), 7.83 (d, | |
| | aminopyrazolo[5,1-f] | J = 7.8 Hz, 1H), 7.62 (d, J = 8.7 Hz, 2H), 7.40 (d, | |
| | [1,2,4]triazin-5-yl)-N- | J = 8.7 Hz, 2H), 7.19 (s, 1H), 7.06-6.99 (m, | |
| | ethyl-2- | 1H), 6.43 (dd, J = 16.9, 10.1 Hz, 1H), 6.25 (dd, | |
| | methoxybenzamide | J = 17.0, 1.9 Hz, 1H), 5.76 (dd, J = 10.1, 1.9 Hz, | |
| | | 1H), 3.82 (s, 3H), 3.40-3.12 (m, 2H), 1.14 (t, | |
| | | J = 7.2 Hz, 3H). | |
| 61 | 4-(6-(4- | ¹H NMR (400 MHz, DMSO) δ 10.23 (s, 1H), | 472 |
| | acrylamidophenyl)-4- | 8.16 (s, 1H), 7.98 (d, J = 7.7 Hz, 1H), 7.78 (d, | |
| | aminopyrazolo[5,1-f] | J = 7.7 Hz, 1H), 7.62 (d, J = 8.6 Hz, 2H), 7.41 (d, | |
| | [1,2,4]triazin-5-yl)-N- | J = 8.6 Hz, 2H), 7.20 (s, 1H), 7.02 (d, J = 7.7 Hz, | |
| | isopropyl-2- | 1H), 6.43 (dd, J = 17.0, 10.1 Hz, 1H), 6.30-6.20 | |
| | methoxybenzamide | (m, 1H), 5.80-5.72 (m, 1H), 4.10 (d, J = 6.8 Hz, | |
| | | 1H), 3.82 (s, 3H), 1.19 (d, J = 6.6 Hz, 6H). | |
| 62 | 4-(6-(4- | ¹H NMR (400 MHz, DMSO) δ 10.23 (s, 1H), | 501 |
| | acrylamidophenyl)-4- | 8.36 (t, J = 5.2 Hz, 2H), 8.16 (s, 1H), 7.90 (d, J = | |
| | aminopyrazolo[5,1-f] | 7.9 Hz, 1H), 7.62 (d, J = 8.7 Hz, 2H), 7.40 (d, | |
| | [1,2,4]triazin-5-yl)-N- | J = 8.7 Hz, 2H), 7.21 (s, 1H), 7.03 (dd, J = 7.9, 1.0 | |
| | (2-(dimethylamino) | Hz, 1H), 6.43 (dd, J = 17.0, 10.1 Hz, 1H), 6.25 | |
| | ethyl)-2- | (dd, J = 17.0, 1.9 Hz, 1H), 5.76 (dd, J = 10.1, 1.9 | |
| | methoxybenzamide | Hz, 2H), 3.84 (s, 3H), 3.40 (dd, J = 12.0, 6.3 Hz, | |
| | | 2H), 2.43 (t, J = 6.5 Hz, 2H), 2.22 (s, 6H). | |
| 63 | 4-(6-(4- | ¹H NMR (400 MHz, DMSO) δ 10.22 (s, 1H), | 514 |
| | acrylamidophenyl)-4- | 8.33 (d, J = 7.8 Hz, 1H), 8.16 (s, 1H), 7.73 (d, | |
| | aminopyrazolo[5,1-f] | J = 8.0 Hz, 1H), 7.62 (d, J = 8.4 Hz, 2H), 7.41 (d, | |
| | [1,2,4]triazin-5-yl)-2- | J = 8.8 Hz, 2H), 7.19 (s, 1H), 7.02 (m, 1H), 6.46- | |
| | methoxy-N-((1s,3s)-3- | 6.36 (m, 1H), 6.25 (m, 1H), 5.76 (m, 1H), 4.04 | |
| | methoxycyclobutyl)benzamide | (m, 1H), 3.82 (s, 3H), 3.62 (m, 1H), 3.15 (s, 3H), | |
| | | 2.66-2.56 (m, 2H), 1.91 (m, 2H). | |
| 64 | 4-(2-(4- | ¹H NMR (400 MHz, DMSO) δ 10.31 (s, 1H), | 523 |
| | acrylamidophenyl)-4- | 8.21 (s, 1H), 8.11 (t, J = 5.6 Hz, 1H), 7.67 (m, | |
| | amino-7-cyano-1- | 3H), 7.30 (d, J = 8.4 Hz, 2H), 6.95 (s, 1H), 6.88 | |
| | methyl-1H-pyrrolo | (d, J = 8.0 Hz, 1H), 6.43 (m, 1H), 6.26 (m, 1H), | |
| | [3,2-c] pyridin-3-yl)-N- | 5.77 (d, J = 10.4 Hz, 1H), 3.80 (s, 3H), 3.72 (s, | |
| | isobutyl-2- | 3H), 3.08 (t, J = 6.4 Hz, 2H), 1.80 (m, 1H), 0.88 | |
| | methoxybenzamide | (d, J = 6.4 Hz, 6H). | |
| 65 | 4-(2-(4- | ¹H NMR (400 MHz, DMSO) δ 10.26 (s, 1H), | 491 |
| | acrylamidophenyl)-4- | 8.61 (d, J = 7.5 Hz, 1H), 8.20 (s, 1H), 7.79 (d, | |
| | amino-7-cyano-1- | J = 8.1 Hz, 2H), 7.67 (d, J = 8.5 Hz, 2H), 7.28 (dd, | |
| | methyl-1H-pyrrolo[3,2- | J = 19.5, 8.3 Hz, 4H), 6.42 (dd, J = 17.0, 10.1 | |
| | c]pyridin-3-yl)-N- | Hz, 1H), 6.26 (dd, J = 16.9, 1.8 Hz, 1H), 5.93 (s, | |
| | cyclobutylbenzamide | 1H), 5.77 (d, J = 12.0 Hz, 1H), 4.39 (dd, J = | |
| | | 15.8, 8.0 Hz, 1H), 3.81 (s, 3H), 2.19 (d, J = 7.5 | |
| | | Hz, 2H), 2.03 (dd, J = 20.7, 9.0 Hz, 2H), 1.72- | |
| | | 1.61 (m, 2H). | |
| 66 | 4-(2-(4- | ¹H NMR (400 MHz, DMSO) δ 10.28 (s, 1H), | 521 |
| | acrylamidophenyl)-4- | 8.30-8.18 (m, 2H), 7.69 (d, J = 8.5 Hz, 2H), | |
| | amino-7-cyano-1- | 7.59 (d, J = 7.8 Hz, 1H), 7.30 (d, J = 8.5 Hz, | |
| | methyl-1H-pyrrolo[3,2- | 2H), 6.93 (s, 1H), 6.87 (d, J = 7.9 Hz, 1H), 6.43 | |
| | c]pyridin-3-yl)-N- | (dd, J = 16.9, 10.0 Hz, 1H), 6.32-6.20 (m, 1H), | |
| | cyclobutyl-2- | 5.82-5.73 (m, 1H), 4.37 (dd, J = 16.5, 8.2 Hz, | |
| | methoxybenzamide | 1H), 3.80 (s,3H), 3.72 (s, 3H), 2.23 (dd, J = 29.0, | |
| | | 25.5 Hz,2H), 1.98 (dd, J = 14.7, 6.4 Hz, 2H), | |
| | | 1.65 (m, 2H). | |
| 67 | N-(4-(4-amino-7- | ¹H NMR (400 MHz, DMSO) δ 10.28 (s, 1H), | 488 |
| | cyano-1-methyl-3-(4- | 8.66 (d, J = 4.8 Hz, 2H), 8.19 (s, 1H), 7.71 (d, | |
| | (pyrimidin-2- | J = 8.5 Hz, 2H), 7.34-7.26 (m, 5H), 7.22 (d, J = | |
| | yloxy)phenyl)-1H- | 8.5 Hz, 2H), 6.43 (dd, J = 16.9, 10.1 Hz, 1H), | |
| | pyrrolo[3,2-c]pyridin-2- | 6.27 (d, J = 16.9 Hz, 1H), 5.98 (s, 1H), 5.80- | |
| | yl)phenyl)acrylamide | 5.75 (m, 1H), 3.80 (s, 3H). | |
| 68 | N-(4-(4-amino-7- | ¹H NMR (400 MHz, DMSO) δ 10.30 (br s, 1H), | 518 |
| | cyano-3-(3-methoxy-4- | 8.61 (d, J = 4.8 Hz, 2H), 8.20 (s, 1H), 7.73 (d, | |
| | (pyrimidin-2- | J = 8.5 Hz, 2H), 7.35 (d, J = 8.5 Hz, 2H), 7.23 (t, | |
| | yloxy)phenyl)-1- | J = 4.8 Hz, 1H), 7.19 (d, J = 8.1 Hz, 1H), 7.00 (d, | |
| | methyl-1H-pyrrolo[3,2- | J = 1.6 Hz, 1H), 6.88 (dd, J = 8.0, 1.7 Hz, 1H), | |
| | c]pyridin-2- | 6.48-6.39 (m, 1H), 6.32-6.24 (m, 1H), 5.78 | |
| | yl)phenyl)acrylamide | (dd, J = 10.1, 1.9 Hz, 1H), 3.80 (s, 3H), 3.55 (s, 3H). | |
| 69 | N-(4-(4-amino-7- | ¹H NMR (400 MHz, DMSO) δ 10.28 (s, 1H), | 502 |
| | cyano-1-methyl-3-(4- | 8.46 (d, J = 4.8 Hz, 1H), 8.19 (s, 1H), 7.70 (d, | |
| | ((4-methylpyrimidin-2- | J = 8.4 Hz, 2H), 7.32-7.29 (m, 4H), 7.17 (m, 3H), | |
| | yl)oxy)phenyl)-1H- | 6.43 (m, 1H), 6.27 (m, 1H), 5.78 (m, 1H), 3.81 | |
| | pyrrolo[3,2-c]pyridin-2- | (s, 3H), 2.40 (s, 3H). | |
| | yl)phenyl)acrylamide | | |
| 70 | 4-(4-amino-6-(4- | ¹H NMR (400 MHz, DMSO) δ 9.85 (s, 1H), 8.73 | 526 |
| | methacrylamidophenyl) | (t, J = 6.5 Hz, 1H), 8.16 (s, 1H), 7.83 (d, J = 7.9 | |
| | pyrazolo[5,1- | Hz, 1H), 7.64 (d, J = 8.7 Hz, 2H), 7.37 (d, J = | |
| | f][1,2,4]triazin-5-yl)-2- | 8.6 Hz, 2H), 7.24 (s, 1H), 7.03 (d, J = 7.9 Hz, | |
| | methoxy-N-(2,2,2- | 1H), 5.78 (s, 1H), 5.52 (s, 1H), 4.15 (m, 2H), | |
| | trifluoroethyl)benzamide | 3.84 (s, 3H), 1.93 (s, 3H). | |
| 72 | (E)-4-(4-amino-6-(4-(4- | ¹H NMR (400 MHz, DMSO) δ 10.13 (s, 1H), | 569 |
| | (dimethylamino)but-2- | 8.73 (t, J = 6.5 Hz, 1H), 8.18 (d, J = 13.2 Hz, | |
| | enamido)phenyl)pyrazolo[5,1- | 2H), 7.84 (d, J = 7.9 Hz, 1H), 7.61 (d, J = 8.7 | |
| | f][1,2,4]triazin- | Hz, 2H), 7.38 (d, J = 8.7 Hz, 2H), 7.24 (s, 1H), | |
| | 5-yl)-2-methoxy-N- | 7.04 (d, J = 7.8 Hz, 1H), 6.77-6.65 (m, 1H), | |
| | (2,2,2- | 6.26 (d, J = 15.4 Hz, 1H), 4.15 (dd, J = 9.7, 6.5 | |
| | trifluoroethyl)benzamide | Hz, 2H), 3.84 (s, 3H), 3.05 (d, J = 5.0 Hz, 2H), | |
| | | 2.17 (s, 6H). | |
| 73 | 4-(6-(4- | ¹H NMR (400 Hz, MeOD) δ 8.14 (s, 1H), 7.73 | 538 |
| | acrylamidophenyl)-4- | (d, J = 8.0 Hz, 1H), 7.64 (d, J = 8.0 Hz, 2H), | |
| | aminopyrazolo[5,1- | 7.54 (dd, J = 4.0, 4.0 Hz, 1H), 7.46-7.42 (m, | |
| | f][1,2,4]triazin-5-yl)-N- | 3H), 6.48-6.35 (m, 2H), 5.79 (dd, J = 4.0, 4.0 | |
| | cyclobutyl-2- | Hz, 1H), 4.55-4.51 (m, 1H), 2.41-2.39 (m, 2H), | |
| | (trifluoromethoxy)benzamide | 2.33-2.32 (m, 2H), 1.87-1.80(m, 2H). | |
| 74 | 4-(6-(4- | ¹H NMR (400 MHz, DMSO) δ 10.23 (s, 1H), | 468 |
| | acrylamidophenyl)-4- | 8.59 (d, J = 7.8 Hz, 1H), 8.16 (s, 1H), 7.62 (d, | |
| | aminopyrazolo[5,1- | J = 8.6 Hz, 2H), 7.43 (dd, J = 13.0, 8.2 Hz, 3H), | |
| | f][1,2,4]triazin-5-yl)-N- | 7.33-7.24 (m, 2H), 6.42 (dd, J = 17.0, 10.1 Hz, | |
| | cyclobutyl-2- | 1H), 6.25 (dd, J = 17.0, 1.9 Hz, 1H), 5.79-5.74 | |
| | methylbenzamide | (m, 1H), 4.40 (dd, J = 16.1, 8.2 Hz, 1H), 2.35 (s, | |
| | | 3H), 2.24 (d, J = 7.8 Hz, 2H), 2.08-1.97 (m, | |
| | | 2H), 1.73-1.64 (m, 2H). | |
| 75 | 4-(6-(4- | ¹H NMR (400 MHz, DMSO) δ 10.25 (s, 1H), | 498 |
| | acrylamidophenyl)-4- | 8.38 (d, J = 7.7 Hz, 1H), 8.16 (s, 1H), 7.78 (d, | |
| | aminopyrazolo[5,1- | J = 7.7 Hz, 1H), 7.62 (d, J = 8.2 Hz, 2H), 7.40 (d, | |
| | f][1,2,4]triazin-5-yl)-N- | J = 8.2 Hz, 2H), 7.19 (s, 1H), 7.02 (d, J = 7.8 Hz, | |
| | cyclobutyl-2- | 1H), 6.43 (dd, J = 16.8, 10.1 Hz, 1H), 6.25 (d, | |
| | ethoxybenzamide | J = 16.9 Hz, 1H), 5.76 (d, J = 10.4 Hz, 1H), 4.44 | |
| | | (dd, J = 15.7, 7.8 Hz, 1H), 4.10 (d, J = 6.8 Hz, | |
| | | 2H), 2.37-2.16 (m, 3H), 2.06-1.90 (m, 3H), 1.36 | |
| | | (t, J = 6.6 Hz, 3H). | |
| 76 | 4-(6-(4- | ¹H NMR (400 MHz, DMSO) δ 10.22 (s, 1H), | 498 |
| | acrylamidophenyl)-4- | 8.35 (d, J = 7.8 Hz, 1H), 8.15 (s, 1H), 7.67-7.56 | |
| | aminopyrazolo[5,1- | (m, 3H), 7.40 (d, J = 8.6 Hz, 2H), 7.17 (s, 1H), | |
| | f][1,2,4]triazin-5-yl)-N- | 6.48-6.37 (m, 1H), 6.25 (dd, J = 17.0, 1.8 Hz, | |
| | cyclobutyl-2-methoxy- | 1H), 5.76 (dd, J = 10.2, 1.8 Hz, 1H), 4.44 (dd, | |
| | 5-methylbenzamide | J = 15.9, 7.7 Hz, 1H), 3.82 (s, 3H), 2.29-2.21 (m, | |
| | | 2H), 2.10-1.99 (m, 2H), 1.89 (s, 3H), 1.74-1.63 | |
| | | (m, 2H). | |
| 77 | 4-(4-amino-6-(4-(2- | ¹H NMR (400 MHz, DMSO) δ 10.51 (s, 1H), | 580 |
| | (trifluoromethyl)acryl- | 8.73 (t, J = 6.4 Hz, 1H), 8.33 (s, 1H), 8.17 (s, | |
| | amido)phenyl)pyrazolo[5,1- | 1H), 7.84 (d, J = 7.8 Hz, 1H), 7.61 (d, J = 8.5 | |
| | f][1,2,4]triazin-5- | Hz, 2H), 7.42 (d, J = 8.5 Hz, 2H), 7.25 (s, 1H), | |
| | yl)-2-methoxy-N- | 7.03 (d, J = 7.9 Hz, 1H), 6.53 (d, J = 14.4 Hz, | |
| | (2,2,2- | 2H), 5.77 (s, 1H), 4.15 (dd, J = 9.6, 6.6 Hz, 2H), | |
| | trifluoroethyl)benzamide | 3.85 (s, 3H). | |
| 78 | 4-(4-amino-6-(4-(N- | ¹H NMR (400 MHz, DMSO) δ 8.74 (d, J = 6.2 | 526 |
| | methylacrylamido)phe- | Hz, 1H), 8.37 (s, 1H), 8.19 (s, 1H), 7.88 (d, J = | |
| | nyl)pyrazolo[5,1-f] | 7.9 Hz, 1H), 7.52 (d, J = 8.4 Hz, 2H), 7.27 (d, | |
| | [1,2,4]triazin-5-yl)-2- | J = 6.8 Hz, 3H), 7.09 (d, J = 7.8 Hz, 1H), 6.13 (d, | |
| | methoxy-N-(2,2,2- | J = 2.6 Hz, 2H), 5.76 (s, 1H), 5.58 (d, J = 12.4 | |
| | trifluoroethyl)benzamide | Hz, 1H), 4.15 (dd, J = 9.6, 6.6 Hz, 2H), 3.85 (s, | |
| | | 3H), 3.23 (s, 3H). | |
| 79 | 4-(6-(4- | ¹H NMR (400 MHz, DMSO) δ 10.23 (s, 1H), | 526 |
| | acrylamidophenyl)-4- | 8.16 (s, 1H), 7.61 (t, J = 7.8 Hz, 2H), 7.37 (t, J = | |
| | aminopyrazolo[5,1-f] | 7.7 Hz, 2H), 7.26 (dd, J = 12.3, 7.6 Hz, 1H), 7.18 | |
| | [1,2,4]triazin-5-yl)-2- | (s, 1H), 7.01 (t, J = 8.5 Hz, 1H), 6.43 (dd, J = | |
| | methoxy-N-methyl-N- | 16.9, 10.1 Hz, 1H), 6.25 (d, J = 16.2 Hz, 1H), | |
| | (2,2,2-trifluoroethyl) | 5.76 (d, J = 11.1 Hz, 1H), 4.24 (d, J = 104.3 Hz, | |
| | benzamide | 2H), 3.75 (s, 3H), 3.10 (s, 1H), 2.98 (s, 2H). | |
| 80 | N-(4-(4-amino-5-(3- | ¹H NMR (400 MHz, DMSO) δ 10.23 (s, 1H), | 522 |
| | methoxy-4-(4- | 9.05 (d, J = 1.6 Hz, 1H), 8.17 (s, 1H), 7.97 (d, | |
| | (trifluoromethyl)oxazol- | J = 8.0 Hz, 1H), 7.63 (d, J = 8.8 Hz, 2H), 7.41 (d, | |
| | 2-yl)phenyl)pyrazolo[5,1- | J = 8.8 Hz, 2H), 7.32 (s, 1H), 7.08 (m, 1H), 6.43 | |
| | f][1,2,4]triazin-6- | (m, 1H), 6.25 (m, 1H), 5.76 (m, 1H), 3.85 (s, 3H). | |
| | yl)phenyl)acrylamide | | |
| 81 | 4-(6-(4- | ¹H NMR (400 MHz, DMSO) δ 10.22 (s, 1H), | 486 |
| | acrylamidophenyl)-4- | 8.40-8.14 (m, 1H), 7.79 (d, J = 7.8 Hz, 1H), | |
| | aminopyrazolo[5,1-f] | 7.62 (d, J = 8.7 Hz, 2H), 7.41 (d, J = 8.7 Hz, | |
| | [1,2,4]triazin-5-yl)-N- | 2H), 7.21 (s, 1H), 7.17 (d, J = 7.8 Hz, 1H), 7.03 | |
| | isobutyl-2- | (d, J = 7.8 Hz, 1H), 6.43 (dd, J = 17.0, 10.1 Hz, | |
| | methoxybenzamide | 1H), 6.25 (dd, J = 17.0, 1.9 Hz, 1H), 5.76 (dd, | |
| | | J = 10.1, 1.9 Hz, 1H), 3.82 (s, 3H), 3.14 (t, J = 6.4 | |
| | | Hz, 2H), 1.91-1.79 (m, 1H), 0.92 (d, J = 6.7 Hz, 6H). | |
| 82 | N-(4-(4-amino-5-(2,2- | ¹H NMR (400 MHz, MeOD) δ 8.13 (s, 1H), 8.00 | 456 |
| | dimethyl-4-oxo-3,4- | (d, J = 7.6 Hz, 1H), 7.63 (d, J = 8.8 Hz, 2H), | |
| | dihydro-2H-benzo[e] | 7.45 (d, J = 8.8 Hz, 2H), 7.21 (m, 1H), 7.01 (s, | |
| | [1,3]oxazin-7- | 1H), 6.41 (m, 2H), 5.79 (m, 1H), 1.66 (s, 6H). | |
| | yl)pyrazolo[5,1-f] | | |
| | [1,2,4]triazin-6-yl) | | |
| | phenyl)acrylamide | | |
| 83 | N-(4-(4-amino-5-(5- | ¹H NMR (400 Hz, DMSO) δ 10.23 (s, 1H), 8.41 | 442 |
| | oxo-2,3,4,5- | (t, J = 8.0 Hz, 1H), 8.15(s, 1H), 7.91 (d, J = 8.0 | |
| | tetrahydrobenzo[f][1,4] | Hz, 1H), 7.62 (d, J = 12 Hz, 2H), 7.37 (d, J = 8 | |
| | oxazepin-8-yl) | Hz, 2H), 7.11 (d, J = 8 Hz, 1H), 7.01 (d, J = 4 | |
| | pyrazolo[5,1-f] | Hz, 1H), 6.46-6.39 (m, 1H), 6.24 (dd, J = 1.6, | |
| | [1,2,4]triazin-6-yl) | 2.0 Hz, 1H), 5.76 (dd, J = 12, 4.0 Hz, 1H), 4.32 | |
| | phenyl)acrylamide | (d, J = 8 Hz, 2H), 3.41 (dd, J = 4.0, 2.0 Hz, 2H). | |
| 84 | 4-(6-(4- | ¹H NMR (400 MHz, MeOD) δ 8.07 (s, 1H), 7.68- | 498 |
| | acrylamidophenyl)-4- | 7.56 (m, 2H), 7.55-7.47 (m, 2H), 6.94 (s, 2H), | |
| | aminopyrazolo[5,1-f] | 6.47-6.31 (m, 2H), 5.77 (m, 1H), 4.61-4.42 | |
| | [1,2,4]triazin-5-yl)-N- | (m, 1H), 3.75 (s, 3H), 2.45-2.34 (m, 2H), 2.31 | |
| | cyclobutyl-2-methoxy- | (s, 3H), 2.10-2.02 (m, 2H), 1.91-1.73 (m, 2H). | |
| | 6-methylbenzamide | | |
| 85 | 4-(6-(4-acrylamido-2- | ¹H NMR (400 MHz, DMSO) δ 10.26 (s, 1H), | 542 |
| | methoxyphenyl)-4- | 8.63 (t, J = 6.4 Hz, 1H), 8.16 (s, 1H), 7.76 (d, J = | |
| | aminopyrazolo[5,1-f] | 7.9 Hz, 1H), 7.43 (s, 1H), 7.31-7.23 (m, 2H), | |
| | [1,2,4]triazin-5-yl)-2- | 7.01 (d, J = 1.1 Hz, 1H), 6.88 (dd, J = 7.9, 1.3 | |
| | methoxy-N-(2,2,2- | Hz, 1H), 6.53-6.40 (m, 1H), 6.27 (dd, J = 17.0, | |
| | trifluoroethyl)benzamide | 2.0 Hz, 1H), 5.78 (dd, J = 10.1, 1.9 Hz, 1H), 4.18- | |
| | | 4.03 (m, 2H), 3.75 (s, 3H), 3.34 (s, 3H). | |
| 86 | 4-(6-(4-acrylamido-2- | ¹H NMR (400 MHz, MeOD) δ 8.14 (s, 1H), 7.94 | 530 |
| | fluorophenyl)-4- | (d, J = 7.9 Hz, 1H), 7.63 (dd, J = 12.3, 1.8 Hz, | |
| | aminopyrazolo[5,1-f] | 1H), 7.43 (t, J = 8.1 Hz, 1H), 7.36 (dd, J = 8.5, | |
| | [1,2,4]triazin-5-yl)-2- | 1.9 Hz, 1H), 7.07 (dd, J = 11.9, 4.0 Hz, 2H), 6.39 | |
| | methoxy-N-(2,2,2- | (dd, J = 5.9, 3.4 Hz, 2H), 5.80 (dd, J = 8.2, 3.6 | |
| | trifluoroethyl)benzamide | Hz, 1H), 4.13 (m, 2H), 3.84 (s, 3H). | |
| 87 | 4-(6-(4-acrylamido-2- | ¹H NMR (400 MHz, DMSO) δ 10.16 (s, 1H), | 526 |
| | methylphenyl)-4- | 8.63 (t, J = 6.5 Hz, 1H), 8.19 (s, 1H), 7.73 (d, J = | |
| | aminopyrazolo[5,1-f] | 7.8 Hz, 1H), 7.55-7.45 (m, 2H), 7.16 (d, J = 8.2 | |
| | [1,2,4]triazin-5-yl)-2- | Hz, 1H), 7.01 (s, 1H), 6.89 (d, J = 7.9 Hz, 1H), | |
| | methoxy-N-(2,2,2- | 6.43 (dd, J = 16.9, 10.3 Hz, 1H), 6.25 (d, J = | |
| | trifluoroethyl)benzamide | 15.5 Hz, 1H), 5.76 (d, J = 11.2 Hz, 1H), 4.10 | |
| | | (dd, J = 9.6, 6.6 Hz, 2H), 3.73 (s, 3H), 1.99 (s, 3H). | |
| 88 | 4-(6-(2-acryloyl-2- | ¹H NMR (400 MHz, DMSO) δ 8.71 (t, J = 6.4 | 516 |
| | azaspiro[3.3]heptan-6- | Hz, 1H), 8.11 (s, 1H), 7.89 (d, J = 8.0 Hz, 1H), | |
| | yl)-4- | 7.11 (s, 1H), 7.03 (d, J = 8.0 Hz, 1H), 6.27 (m, | |
| | aminopyrazolo[5,1-f] | 1H), 6.07 (m, 1H), 5.64 (d, J = 10.0 Hz, 1H), | |
| | [1,2,4]triazin-5-yl)-2- | 4.29 (s, 1H), 4.15 (m, 3H), 3.99 (s, 1H), 3.93 (s, | |
| | methoxy-N-(2,2,2- | 3H), 3.87 (s, 1H), 3.51-3.43 (m, 1H), 2.53 (s, 4H). | |
| | trifluoroethyl)benzamide | | |
| 89 | 4-(6-(4- | ¹H NMR (400 MHz, DMSO) δ 10.22 (s, 1H), | 470 |
| | acrylamidophenyl)-4- | 8.16 (d, J = 3.1 Hz, 2H), 7.74 (d, J = 7.8 Hz, | |
| | aminopyrazolo[5,1-f] | 1H), 7.62 (d, J = 8.6 Hz, 2H), 7.40 (d, J = 8.6 | |
| | [1,2,4]triazin-5-yl)-N- | Hz, 2H), 7.18 (s, 1H), 7.02 (d, J = 7.8 Hz, 1H), | |
| | cyclopropyl-2- | 6.43 (dd, J = 17.0, 10.2 Hz, 1H), 6.25 (d, J = | |
| | methoxybenzamide | 15.3 Hz, 1H), 5.76 (d, J = 11.7 Hz, 1H), 3.79 (s, | |
| | | 3H), 2.85 (m, 1H), 0.72 (m, 2H), 0.61-0.54 (m, 2H). | |
| 90 | 4-(6-(6- | ¹H NMR (400 Hz, DMSO) δ 10.79 (s, 1H), 8.73 | 513 |
| | acrylamidopyridin-3- | (t, J = 6.6 Hz, 1H), 8.33 (d, J = 1.8 Hz, 1H), 8.19 | |
| | yl)-4-aminopyrazolo[5,1- | (s, 1H), 8.16 (d, J = 8.8 Hz, 1H), 7.87-7.79 (m, | |
| | f] [1,2,4]triazin-5- | 2H), 7.27 (s, 1H), 7.07 (d, J = 7.9 Hz, 1H), 6.58 | |
| | yl)-2-methoxy-N- | (dd, J = 17.0, 10.2 Hz, 1H), 6.31 (dd, J = 17.0, | |
| | (2,2,2-trifluoroethyl) | 1.8 Hz, 1H), 5.79 (dd, J = 10.1, 1.8 Hz, 1H), 4.14 | |
| | benzamide | m, 2H), 3.85 (s, 3H). | |
| 91 | 4-(6-(4- | ¹H NMR (400 MHz, DMSO) δ 10.25 (s, 1H), | 548 |
| | acrylamidophenyl)-4- | 8.98 (t, J = 6.4 Hz, 1H), 8.43 (s, 1H), 8.18 (s, | |
| | aminopyrazolo[5,1-f] | 1H), 7.64 (d, J = 7.9 Hz, 3H), 7.39 (d, J = 8.7 | |
| | [1,2,4]triazin-5-yl)-2- | Hz, 2H), 7.36-6.95 (m, 3H), 6.43 (dd, J = 17.0, | |
| | (difluoromethoxy)-N- | 10.1 Hz, 1H), 6.26 (dd, J = 17.0, 1.9 Hz, 1H), | |
| | (2,2,2-trifluoroethyl) | 5.77 (dd, J = 10.1, 1.9 Hz, 2H), 4.18-4.02 (m, 2H). | |
| | benzamide | | |
| 92 | 4-(4-amino-6-(4-(2- | ¹H NMR (400 MHz, DMSO) δ 10.36 (d, J = 4.9 | 544 |
| | fluoroacrylamido)phe- | Hz, 1H), 8.34 (s, 1H), 8.16 (d, J = 2.1 Hz, 1H), | |
| | nyl)pyrazolo[5,1- | 7.67 (dd, J = 8.7, 4.9 Hz, 2H), 7.42-7.35 (m, | |
| | f][1,2,4]triazin-5-yl)-2- | 2H), 7.26 (dd, J = 12.9, 7.7 Hz, 1H), 7.18 (s, | |
| | methoxy-N-methyl-N- | 1H), 7.01 (dd, J = 12.4, 4.6 Hz, 1H), 5.84 (s, | |
| | (2,2,2-trifluoroethyl) | 1H), 5.71 (dd, J = 47.7, 3.7 Hz, 1H), 5.43 (dd, | |
| | benzamide | J = 15.6, 3.6 Hz, 1H), 4.23 (d, J = 94.5 Hz, 2H), | |
| | | 3.75 (d, J = 2.1 Hz, 3H), 3.04 (d, J = 50.0 Hz, | |
| | | 3H). | |
| 93 | 4-(4-amino-6-(4-(2- | ¹H NMR (400 MHz, DMSO) δ 10.36 (s, 1H), | 516 |
| | fluoroacrylamido)phe- | 8.17 (s, 1H), 8.08 (d, J = 7.3 Hz, 1H), 7.76 (d, | |
| | nyl)pyrazolo[5,1-f] | J = 7.8 Hz, 1H), 7.68 (d, J = 8.7 Hz, 2H), 7.42 (d, | |
| | [1,2,4]triazin-5-yl)-N- | J = 8.7 Hz, 2H), 7.20 (m, J = 0.9 Hz, 1H), 7.02 | |
| | cyclopentyl-2- | (dd, J = 7.8, 1.3 Hz, 1H), 5.71 (dd, J = 47.7, 3.7 | |
| | methoxybenzamide | Hz, 1H), 5.44 (dd, J = 15.6, 3.7 Hz, 1H), 4.21- | |
| | | 4.26 (m, J = 13.2, 6.6 Hz, 1H), 3.82 (s, 3H), | |
| | | 1.99-1.82 (m, 2H), 1.78-1.64 (m, 2H), 1.62-1.45 | |
| | | (m, 4H). | |
| 94 | 4-(4-amino-6-(4-(2- | ¹H NMR (400 MHz, DMSO) δ 10.36 (s, 1H), | 476 |
| | fluoroacrylamido)phe- | 8.37 (s, 1H), 8.25 (t, J = 5.7 Hz, 1H), 8.17 (s, | |
| | nyl)pyrazolo[5,1-f][1,2,4] | 1H), 7.83 (d, J = 7.8 Hz, 1H), 7.68 (d, J = 8.8 | |
| | triazin-5-yl)-N-ethyl-2- | Hz, 2H), 7.42 (d, J = 8.7 Hz, 2H), 7.20 (d, J = | |
| | methoxybenzamide | 1.1 Hz, 1H), 7.02 (dd, J = 7.8, 1.3 Hz, 1H), 5.71 | |
| | | (dd, J = 47.7, 3.7 Hz, 2H), 5.44 (dd, J = 15.6, 3.7 | |
| | | Hz, 1H), 3.82 (s, 3H), 3.32 (d, J = 7.1 Hz, 1H), | |
| | | 3.30 (d, J = 7.1 Hz, 1H), 1.14 (t, J = 7.2 Hz, 3H). | |
| 95 | 4-(4-amino-6-(4-(2- | ¹H NMR (400 MHz, DMSO) δ 10.36 (s, 1H), | 502 |
| | fluoroacrylamido)phe- | 8.34 (d, J = 7.6 Hz, 1H), 8.17 (s, 1H), 7.74 (d, | |
| | nyl)pyrazolo[5,1-f][1,2,4] | J = 7.6 Hz, 1H), 7.68 (d, J = 8.4 Hz, 2H), 7.42 (d, | |
| | triazin-5-yl)-N- | J = 8.8 Hz, 2H), 7.20 (d, J = 1.2 Hz, 1H), 7.02 | |
| | cyclobutyl-2- | (m, 1H), 5.57 (m, 1H), 5.46-5.41 (m, 1H), 4.43 | |
| | methoxybenzamide | (m, 1H), 3.82 (s, 3H), 2.30-2.20 (m, 2H), 2.10- | |
| | | 1.98 (m, 2H), 1.74-1.63 (m, 2H). | |
| 97 | 4-(4-amino-7-cyano-2- | ¹H NMR (400 MHz, DMSO) δ 10.41 (s, 1H), | 509 |
| | (4-(2- | 8.63 (d, J = 7.6 Hz, 1H), 8.21 (s, 1H), 7.77 (m, | |
| | fluoroacrylamido)phe- | 4H), 7.34-7.27 (m, 4H), 6.27-5.32 (m, 4H), | |
| | nyl)-1-methyl-1H- | 4.39 (m, 1H), 3.81 (s, 3H), 2.24-2.15 (m, 2H), | |
| | pyrrolo[3,2-c]pyridin-3- | 2.09-1.99 (m, 2H), 1.66 (m, 2H). | |
| | yl)-N- | | |
| | cyclobutylbenzamide | | |
| 98 | 4-(6-(4- | ¹H NMR (400 MHz, DMSO) δ 10.23 (s, 1H), | 520 |
| | acrylamidophenyl)-4- | 8.58 (d, J = 6.4 Hz, 1H), 8.16 (s, 1H), 7.76 (d, | |
| | aminopyrazolo[5,1-f] | J = 7.6 Hz, 1H), 7.62 (d, J = 8.8 Hz, 2H), 7.40 (d, | |
| | [1,2,4]triazin-5-yl)-N- | J = 8.8 Hz, 2H), 7.21 (s, 1H), 7.04 (d, J = 8.4 Hz, | |
| | (3,3-difluorocyclobutyl)- | 1H), 6.43 (m, 1H), 6.25 (m, 1H), 5.76 (m, 1H), | |
| | 2-methoxybenzamide | 4.35-4.22 (m, 1H), 3.82 (s, 3H), 3.02-2.90 (m, | |
| | | 2H), 2.85-2.72 (m, 2H). | |
| 99 | 4-(4-amino-6-(4-(2- | ¹H NMR (400 MHz, DMSO) δ 10.36 (s, 1H), | 538 |
| | fluoroacrylamido)phe- | 8.58 (d, J = 6.8 Hz, 1H), 8.38 (s, 1H), 8.17 (s, | |
| | nyl)pyrazolo[5,1-f] | 1H), 7.75 (d, J = 8.0 Hz, 1H), 7.68 (d, J = 8.8 | |
| | [1,2,4]triazin-5-yl)-N- | Hz, 2H), 7.42 (d, J = 8.4 Hz, 2H), 7.21 (s, 1H), | |
| | (3,3-difluorocyclobutyl)- | 7.07-6.99 (m, 1H), 5.71 (m, 1H), 5.44 (m, 1H), | |
| | 2-methoxybenzamide | 4.38-4.19 (m, 1H), 3.82 (s, 3H), 3.07-2.87 (m, | |
| | | 2H), 2.77 (m, 2H). | |
| 101 | 4-(4-amino-6-(4-(2- | ¹H NMR (400 MHz, DMSO) δ 10.29 (s, 1H), | 544 |
| | fluoroacrylamido)-2- | 8.66-8.27 (m, 2H), 8.19 (s, 1H), 7.73 (d, J = 7.6 | |
| | methylphenyl)pyrazolo[ | Hz, 1H), 7.60 (s, 1H), 7.55-7.52 (m, 1H), 7.18 | |
| | 5,1-f][1,2,4]triazin-5- | (d, J = 8.4 Hz, 1H), 7.01 (d, J = 1.2 Hz, 1H), | |
| | yl)-2-methoxy-N- | 6.88 (m, 1H), 6.30-5.62 (m, 2H), 5.43 (m, 1H), | |
| | (2,2,2-trifluoroethyl) | 4.15-4.04 (m, 2H), 3.73 (s, 3H), 2.00 (s, 3H). | |
| | benzamide | | |
| 102 | 4-(4-amino-6-(4-(2- | ¹H NMR (400 MHz, DMSO) δ 10.36 (s, 1H), | 560 |
| | fluoroacrylamido)-2- | 8.63 (t, J = 6.4 Hz, 1H), 8.16 (s, 1H), 7.76 (d, J = | |
| | methoxyphenyl)pyrazolo[5,1- | 8.0 Hz, 1H), 7.45-7.41 (m, 2H), 7.31 (d, J = 8.8 | |
| | f][1,2,4]triazin-5- | Hz, 1H), 7.01 (s, 1H), 6.88 (d, J = 8.8 Hz, 1H), | |
| | yl)-2-methoxy-N- | 5.72 (m, 1H), 5.45 (m, 1H), 4.10 (m, 2H), 3.75 | |
| | (2,2,2-trifluoroethyl) | (s, 3H), 3.34 (s, 3H). | |
| | benzamide | | |
| 103 | 4-(4-amino-6-(4-(2- | ¹H NMR (400 MHz, DMSO) δ 10.36 (s, 1H), | 488 |
| | fluoroacrylamido)phe- | 8.16 (d, J = 6.0 Hz, 2H), 7.71 (m, 3H), 7.42 (d, | |
| | nyl)pyrazolo[5,1-f] | J = 8.8 Hz, 2H), 7.18 (s, 1H), 7.02 (d, J = 8.0 Hz, | |
| | [1,2,4]triazin-5-yl)-N- | 1H), 5.71 (m, 1H), 5.44 (m, 1H), 3.79 (s, 3H), | |
| | cyclopropyl-2- | 2.85 (m, 1H), 0.71 (m, 2H), 0.57 (m, 2H). | |
| | methoxybenzamide | | |
| 104 | 4-(4-amino-7-cyano-2- | ¹H NMR (400 MHz, DMSO) δ 10.41 (s, 1H), | 525 |
| | (4-(2-fluoroacrylamido) | 8.21 (s, 1H), 8.07 (d, J = 4.0 Hz, 1H), 7.76 (d, | |
| | phenyl)-1-methyl-1H- | J = 8.4 Hz, 2H), 7.58 (d, J = 8.0 Hz, 1H), 7.33 (d, | |
| | pyrrolo[3,2-c]pyridin-3- | J = 8.4 Hz, 2H), 6.95-6.83 (m, 2H), 6.58-5.35 | |
| | yl)-N-cyclopropyl-2- | (m, 4H), 3.80 (s, 3H), 3.69 (s, 3H), 2.80 (m, 1H), | |
| | methoxybenzamide | 0.73-0.60 (m, 2H), 0.58-0.45 (m, 2H). | |
| 105 | 4-(2-(4-acryl- | ¹H NMR (400 MHz, DMSO) δ 10.28 (s, 1H), | 507 |
| | amidophenyl)-4-amino- | 8.21 (s, 1H), 8.06 (d, J = 4.4 Hz, 1H), 7.69 (d, | |
| | 7-cyano-1-methyl-1H- | J = 8.4 Hz, 2H), 7.58 (d, J = 7.6 Hz, 1H), 7.30 (d, | |
| | pyrrolo[3,2-c]pyridin-3- | J = 8.4 Hz, 2H), 6.92 (s, 1H), 6.87 (d, J = 7.6 Hz, | |
| | yl)-N-cyclopropyl-2- | 1H), 6.43 (m, 1H), 6.30-6.24 (m, 1H), 6.05 (s, | |
| | methoxybenzamide | 1H), 5.80-5.74 (m, 1H), 3.80 (s, 3H), 3.69 (s, | |
| | | 3H), 2.80 (m, 1H), 0.67 (d, J = 5.2 Hz, 2H), 0.52 | |
| | | (d, J = 2.8 Hz, 2H). | |
| 106 | 4-(2-(4- | ¹H NMR (400 MHz, DMSO) δ 10.27 (s, 1H), | 519 |
| | acrylamidophenyl)-4- | 9.10 (t, J = 6.0 Hz, 1H), 8.21 (s, 1H), 7.85 (d, J = | |
| | amino-7-cyano-1- | 8.0 Hz, 2H), 7.67 (d, J = 8.4 Hz, 2H), 7.34 (d, | |
| | methyl-1H-pyrrolo[3,2- | J = 8.4 Hz, 2H), 7.26 (d, J = 8.8 Hz, 2H), 6.42 (m, | |
| | c]pyridin-3-yl)-N- | 1H), 6.31-5.83 (m, 1H), 5.79-5.75 (m, 1H), | |
| | (2,2,2-trifluoroethyl) | 4.06 (m, 2H), 3.81 (s, 3H). | |
| | benzamide | | |
| 107 | 4-(2-(4- | ¹H NMR (400 MHz, DMSO) δ 10.28 (s, 1H), | 495 |
| | acrylamidophenyl)-4- | 8.21 (s, 1H), 8.15 (t, J = 5.6 Hz, 1H), 7.69 (m, | |
| | amino-7-cyano-1- | 3H), 7.30 (d, J = 8.4 Hz, 2H), 6.94 (d, J = 0.8 | |
| | methyl-1H-pyrrolo[3,2- | Hz, 1H), 6.88 (m, 1H), 6.43 (m, 1H), 6.34-5.82 | |
| | c]pyridin-3-yl)-N-ethyl- | (m, 3H), 5.78 (m, 1H), 3.81 (s, 3H), 3.72 (s, 3H), | |
| | 2-methoxybenzamide | 3.27 (m, 2H), 1.09 (t, J = 7.2 Hz, 3H). | |
| 108 | 4-(2-(4- | ¹H NMR (400 MHz, DMSO) δ 10.28 (s, 1H), | 509 |
| | acrylamidophenyl)-4- | 8.21 (s, 1H), 7.89 (d, J = 7.6 Hz, 1H), 7.69 (d, | |
| | amino-7-cyano-1- | J = 8.8 Hz, 2H), 7.63 (d, J = 7.6 Hz, 1H), 7.30 (d, | |
| | methyl-1H-pyrrolo[3,2- | J = 8.8 Hz, 2H), 6.93 (s, 1H), 6.87 (d, J = 7.6 Hz, | |
| | c]pyridin-3-yl)-N- | 1H), 6.43 (m, 1H), 6.27 (m, 3H), 5.78 (m, 1H), | |
| | isopropyl-2- | 4.04 (m, 1H), 3.80 (s, 3H), 3.72 (s, 3H), 1.14 (d, | |
| | methoxybenzamide | J = 6.8 Hz, 6H). | |
| 109 | 4-(2-(4- | ¹H NMR (400 MHz, DMSO) δ 10.27 (s, 1H), | 535 |
| | acrylamidophenyl)-4- | 8.21 (s, 1H), 7.99 (d, J = 7.2 Hz, 1H), 7.69 (d, | |
| | amino-7-cyano-1- | J = 8.4 Hz, 2H), 7.61 (d, J = 7.6 Hz, 1H), 7.30 (d, | |
| | methyl-1H-pyrrolo[3,2- | J = 8.4 Hz, 2H), 6.93 (s, 1H), 6.87 (d, J = 8.4 Hz, | |
| | c]pyridin-3-yl)-N- | 1H), 6.43 (m, 1H), 6.27 (d, J = 16.8 Hz, 1H), | |
| | cyclopentyl-2- | 5.78 (d, J = 11.6 Hz, 1H), 4.18 (m, 1H), 3.80 (s, | |
| | methoxybenzamide | 3H), 3.71 (s, 3H), 1.84 (m, 2H), 1.65 (m, 2H), | |
| | | 1.57-1.43 (m, 4H). | |
| 110 | 4-(2-(4- | ¹H NMR (400 MHz, DMSO) δ 10.27 (s, 1H), | 549 |
| | acrylamidophenyl)-4- | 8.21 (s, 1H), 7.93 (d, J = 8.0 Hz, 1H), 7.69 (d, | |
| | amino-7-cyano-1- | J = 8.4 Hz, 2H), 7.64 (d, J = 8.0 Hz, 1H), 7.30 (d, | |
| | methyl-1H-pyrrolo[3,2- | J = 8.4 Hz, 2H), 6.94 (s, 1H), 6.87 (d, J = 8.0 Hz, | |
| | c]pyridin-3-yl)-N- | 1H), 6.43 (m, 1H), 6.27 (m, 3H), 5.78 (d, J = | |
| | cyclohexyl-2- | 11.6 Hz, 1H), 3.80 (s, 3H), 3.72 (s, 4H), 1.84- | |
| | methoxybenzamide | 1.54 (m, 5H), 1.36-1.16 (m, 5H). | |
| 111 | 4-(2-(4- | ¹H NMR (400 MHz, DMSO) δ 10.28 (s, 1H), | 546 |
| | acrylamidophenyl)-4- | 8.49 (t, J = 6.4 Hz, 1H), 8.21 (s, 1H), 7.68 (m, | |
| | amino-7-cyano-1- | 3H), 7.30 (d, J = 8.4 Hz, 2H), 6.96 (s, 1H), 6.92- | |
| | methyl-1H-pyrrolo[3,2- | 6.87 (m, 1H), 6.43 (m, 1H), 6.27 (m, 1H), 6.24- | |
| | c]pyridin-3-yl)-N-((1- | 5.80 (m, 2H), 5.78 (m, 1H), 3.81 (s, 3H), 3.74 (s, | |
| | cyanocyclopropyl)methyl)- | 3H), 3.44 (d, J = 6.0 Hz, 2H), 1.18 (s, 2H), 1.11 | |
| | 2-methoxybenzamide | (s, 2H). | |
| 112 | 4-(2-(4-acrylamido-2- | ¹H NMR (400 MHz, DMSO) δ 10.31 (s, 1H), | 579 |
| | methoxyphenyl)-4-amino- | 8.62 (t, J = 6.4 Hz, 1H), 8.20 (s, 1H), 7.69 (d, J = | |
| | 7-cyano-1-methyl-1H- | 8.0 Hz, 1H), 7.61 (s, 1H), 7.19-7.16(m, 1H), 7.04 | |
| | pyrrolo[3,2-c]pyridin-3- | (d, J = 8.0 Hz, 1H), 6.90 (m, 2H), 6.43 (m, 1H), | |
| | yl)-2-methoxy-N-(2,2,2- | 6.30-6.25 (m, 1H), 5.79 (m, 1H), 4.12-4.03 | |
| | trifluoroethyl)benzamide | (m, 2H), 3.73 (m, 6H), 3.69 (s, 3H). | |
| 113 | 5-(4-acrylamidophenyl)- | ¹H NMR (400 MHz, DMSO) δ 10.17 (s, 1H), | 481 |
| | 4-(3-methoxy-4-((6- | 8.61 (d, J = 9.6 Hz, 2H), 7.86 (s, 1H), 7.62 (dt, | |
| | methylpyridin-2-yl) | J = 17.3, 8.1 Hz, 4H), 7.08 (d, J = 8.6 Hz, 2H), | |
| | oxy)phenyl) | 6.99 (d, J = 8.1 Hz, 1H), 6.92 (d, J = 7.3 Hz, | |
| | nicotinamide | 1H), 6.83 (d, J = 1.9 Hz, 1H), 6.73 (dd, J = 8.1, | |
| | | 1.9 Hz, 1H), 6.50 (d, J = 8.2 Hz, 1H), 6.42 (dd, | |
| | | J = 17.0, 10.1 Hz, 1H), 6.25 (dd, J = 17.0, 2.0 Hz, | |
| | | 1H), 5.76 (dd, J = 10.1, 2.0 Hz, 1H), 3.44 (s, | |
| | | 3H), 2.30 (s, 3H). | |
| 114 | 5-(4-acrylamidophenyl)- | ¹H NMR (400 MHz, DMSO) δ 10.17 (s, 1H), | 510 |
| | 2-amino-4-(3- | 7.66-7.52 (m, 5H), 6.90 (dd, J = 12.7, 7.7 Hz, | |
| | methoxy-4-((6- | 6H), 6.73-6.66 (m, 2H), 6.46-6.33 (m, 2H), | |
| | methylpyridin-2- | 6.25 (d, J = 17.1 Hz, 1H), 5.75 (d, J = 11.6 Hz, | |
| | yl)oxy)phenyl)-6- | 1H), 3.43 (s, 3H), 2.25 (s, 6H). | |
| | methylnicotinamide | | |
| 115 | N-(4-(4-amino-3-(3- | ¹H NMR (400 MHz, DMSO) δ 11.34 (s, 1H), | 510 |
| | methoxy-4-((6-methyl- | 10.37(s, 1H), 7.75-7.66 (m, 3H), 7.39 (d, J = 8.0 | |
| | pyridin-2-yl) oxy) | Hz, 2H), 7.24-7.17 (m, 2H), 7.04-6.94 (m, 2H), | |
| | phenyl)-7-oxo-6,7- | 6.70 (d, J = 8.0 Hz, 1H), 6.47-6.25 (m, 2H), | |
| | dihydro-2H-pyrazolo | 5.80-5.77 (m, 1H), 3.58 (s, 3H), 2.28 (s, 3H). | |
| | [3,4-d]pyridazin-2- | | |
| | yl)phenyl)acrylamide | | |
| 116 | N-(4-(4-amino-3-(3- | ¹H NMR (400 MHz, DMSO) δ 11.34 (s, 1H), | 524 |
| | methoxy-4-((6- | 10.00 (s, 1H), 7.77-7.67 (m, 3H), 7.38 (d, J = 8.8 | |
| | methylpyridin-2-yl) | Hz, 2H), 7.25-7.17 (m, 2H), 7.04-6.95 (m, 2H), | |
| | oxy)phenyl)-7-oxo-6,7- | 6.71 (d, J = 8.0 Hz, 1H), 5.81 (s, 1H), 5.55 (s, | |
| | dihydro-2H-pyrazolo | 1H), 4.96 (s, 2H), 3.59 (s, 3H), 2.28 (s, 3H), 1.95 | |
| | [3,4-d]pyridazin-2-yl) | (s, 3H). | |
| | phenyl)methacrylamide | | |
| 117 | 1-(3-((4-amino-5-(3- | ¹H NMR (400 MHz, CDCl₃) δ 8.30 (s, 1H), 7.55 | 499 |
| | methoxy-4-((6- | (t, J = 7.8 Hz, 1H), 7.20 (d, J = 8.1 Hz, 1H), 7.07 | |
| | methylpyridin-2-yl) | (dd, J = 18.6, 9.7 Hz, 3H), 6.86 (d, J = 7.3 Hz, | |
| | oxy)phenyl)-7H- | 1H), 6.62 (d, J = 8.4 Hz, 1H), 6.25 (d, J = 16.4 | |
| | pyrrolo[2,3-d] | Hz, 1H), 5.74-5.41 (m, 2H), 4.45-4.02 (m, | |
| | pyrimidin-7-yl) | 3H), 3.83 (s, 4H), 3.25-2.78 (m, 2H), 2.44 (s, | |
| | methyl)piperidin-1-yl) | 3H), 1.61-1.27 (m, 5H). | |
| | prop-2-en-1-one | | |
| 118 | N-(4-(4-amino-3-(3- | ¹H NMR (400 MHz, DMSO) δ 11.37 (s, 1H), | 528 |
| | methoxy-4-((6-methyl- | 10.12 (s, 1H), 8.17 (t, J = 12.0 Hz, 1H), 7.70 (t, | |
| | pyridin-2-yl)oxy) | J = 7.6 Hz, 1H), 7.45-7.41 (m, 1H), 7.28-7.19 (m, | |
| | phenyl)-7-oxo-6,7- | 3H), 7.08-7.05 (m, 1H), 6.96 (d, J = 7.6 Hz, 1H), | |
| | dihydro-2H-pyrazolo | 6.71 (d, J = 8.4 Hz, 1H), 6.66-6.60 (m, 1H), | |
| | [3,4-d]pyridazin-2-yl)- | 6.32-6.27 (m, 1H), 5.82-5.79 (m, 1H), 4.97 (s, | |
| | 2-fluorophenyl) | 2H), 3.60 (s, 3H), 2.27 (s, 3H). | |
| | acrylamide | | |
| 119 | 5-(4-acrylamidophenyl)- | ¹H NMR (400 MHz, DMSO) δ 11.34 (s, 1H), | 466 |
| | 2-amino-4-(3-methoxy- | 10.36 (s, 1H), 8.22 (dd, J = 4.9, 1.4 Hz, 1H), | |
| | 4-((6-methylpyridin-2- | 7.89 (ddd, J = 8.3, 7.3, 2.0 Hz, 1H), 7.72 (d, J = | |
| | yl)oxy)phenyl)-6- | 8.9 Hz, 2H), 7.48 (d, J = 8.6 Hz, 2H), 7.35 (d, | |
| | methylnicotinamide | J = 8.9 Hz, 2H), 7.22 (d, J = 8.7 Hz, 2H), 7.20- | |
| | | 7.16 (m, 1H), 7.09 (d, J = 8.2 Hz, 1H), 6.42 (dd, | |
| | | J = 17.0, 10.1 Hz, 1H), 6.28 (dd, J = 17.0, 1.9 | |
| | | Hz, 1H), 5.79 (dd, J = 10.0, 2.0 Hz, 1H), 4.85 (s, 2H). | |
| 120 | 4-(4-amino-2-(4- | ¹H NMR (400 MHz, DMSO) δ 11.37 (s, 1H), | 486 |
| | methacrylamidophenyl)- | 9.97 (s, 1H), 8.58 (t, J = 5.8 Hz, 1H), 7.89 (d, J = | |
| | 7-oxo-6,7-dihydro-2H- | 8.4 Hz, 2H), 7.71 (d, J = 9.0 Hz, 2H), 7.54 (d, | |
| | pyrazolo[3,4- | J = 8.4 Hz, 2H), 7.32 (d, J = 8.9 Hz, 2H), 5.79 (s, | |
| | d]pyridazin-3-yl)-N- | 1H), 5.54 (s, 1H), 4.81 (s, 2H), 3.12-3.02 (m, | |
| | isobutylbenzamide | 2H), 1.93 (s, 3H), 1.83 (dt, J = 13.5, 6.7 Hz, 1H), | |
| | | 0.88 (d, J = 6.7 Hz, 6H). | |
| 121 | 4-(2-(4- | ¹H NMR (400 MHz, DMSO) δ 11.37 (s, 1H), | 472 |
| | acrylamidophenyl)-4- | 10.34 (s, 1H), 8.58 (t, J = 5.8 Hz, 1H), 7.89 (d, | |
| | amino-7-oxo-6,7- | J = 8.4 Hz, 2H), 7.69 (d, J = 8.9 Hz, 2H), 7.54 (d, | |
| | dihydro-2H- | J = 8.3 Hz, 2H), 7.33 (d, J = 8.9 Hz, 2H), 6.41 | |
| | pyrazolo[3,4- | (dd, J = 16.9, 10.1 Hz, 1H), 6.27 (dd, J = 17.0, | |
| | d]pyridazin-3-yl)-N- | 2.0 Hz, 1H), 5.78 (dd, J = 10.0, 2.0 Hz, 1H), 4.81 | |
| | isobutylbenzamide | (s, 2H), 3.11-3.03 (m, 2H), 1.83 (dt, J = 13.4, | |
| | | 6.8 Hz, 1H), 0.88 (d, J = 6.7 Hz, 6H). | |
| 122 | N-(4-(4-cyano-6-(3- | ¹H NMR (400 MHz, DMSO) δ 10.23 (s, 1H), | 517 |
| | methoxy-4-((6- | 9.07 (s, 1H), 7.71 (t, J = 8.0 Hz, 3H), 7.33 (d, J = | |
| | methylpyridin-2- | 8.8 Hz, 2H), 7.21-7.17 (m, 2H), 7.05-7.03 (m, | |
| | yl)oxy)phenyl)-7- | 1H), 6.96 (d, J = 7.2 Hz, 1H), 6.70 (d, J = 8.4 | |
| | methyl-7H-pyrrolo[2,3- | Hz, 1H), 6.47-6.41 (m, 1H), 6.29-6.24 (m, 1H), | |
| | d]pyrimidin-5- | 5.78-5.75 (m, 1H), 3.86 (s, 3H), 3.58 (m, 3H), | |
| | yl)phenyl)acrylamide | 2.29 (s, 3H). | |
| 123 | N-(4-(4-chloro-6-(3- | ¹H NMR (400 MHz, DMSO) δ 10.18 (s, 1H), | 526 |
| | methoxy-4-((6- | 8.71 (s, 1H), 7.72-7.58 (m, 3H), 7.25 (d, J = 8.0 | |
| | methylpyridin-2- | Hz, 2H), 7.13 (d, J = 4.4 Hz, 2H), 7.00 (d, J = | |
| | yl)oxy)phenyl)-7- | 7.9 Hz, 1H), 6.94 (d, J = 7.2 Hz, 1H), 6.66 (d, | |
| | methyl-7H-pyrrolo[2,3- | J = 8.1 Hz, 1H), 6.43 (dd, J = 16.9, 10.1 Hz, 1H), | |
| | d]pyrimidin-5- | 6.25 (d, J = 17.2 Hz, 1H), 5.75 (d, J = 9.8 Hz, | |
| | yl)phenyl)acrylamide | 1H), 3.82 (s, 3H), 3.55 (s, 3H), 2.28 (s, 3H). | |
| 124 | N-(4-(4-methoxy-6-(3- | ¹H NMR (400 MHz, DMSO) δ 10.12 (s, 1H), | 522 |
| | methoxy-4-((6- | 8.50 (s, 1H), 7.68 (t, J = 7.7 Hz, 1H), 7.57 (d, J = | |
| | methylpyridin-2- | 8.4 Hz, 2H), 7.20 (d, J = 8.5 Hz, 2H), 7.13 (d, | |
| | yl)oxy)phenyl)-7- | J = 8.4 Hz, 2H), 6.97-6.90 (m, 2H), 6.66 (d, J = | |
| | methyl-7H-pyrrolo[2,3- | 8.1 Hz, 1H), 6.43 (dd, J = 16.9, 10.1 Hz, 1H), | |
| | d]pyrimidin-5- | 6.24 (dd, J = 16.9, 1.6 Hz, 1H), 5.74 (dd, J = | |
| | yl)phenyl)acrylamide | 10.1, 1.6 Hz, 1H), 3.95 (s, 3H), 3.76 (s, 3H), | |
| | | 3.56 (s, 3H), 2.31 (s, 3H). | |
| 125 | 4-(2-(4- | ¹H NMR (400 MHz, DMSO) δ 11.38 (s, 1H), | 470 |
| | acrylamidophenyl)-4- | 10.35 (s, 1H), 8.73 (d, J = 7.5 Hz, 1H), 7.88 (d, | |
| | amino-7-oxo-6,7- | J = 8.4 Hz, 2H), 7.68 (d, J = 9.0 Hz, 2H), 7.53 (d, | |
| | dihydro-2H- | J = 8.4 Hz, 2H), 7.33 (d, 2H), 6.49-6.34 (m, | |
| | pyrazolo[3,4- | 1H), 6.27 (dd, J = 17.0, 2.0 Hz, 1H), 5.79 (dd, | |
| | d]pyridazin-3-yl)-N- | J = 10.0, 2.0 Hz, 1H), 4.82 (s, 2H), 4.48-4.31 (m, | |
| | cyclobutylbenzamide | 1H), 2.28-2.12 (m, 2H), 2.11-1.96 (m, 2H), | |
| | | 1.75-1.59 (m, 2H). | |
| 126 | N-(4-(4-amino-7-oxo-3- | ¹H NMR (400 MHz, DMSO) δ 11.37 (s, 1H), | 470 |
| | (4-(pyrrolidine-1- | 10.35 (s, 1H), 7.69 (d, J = 8.8 Hz, 2H), 7.58 (d, | |
| | carbonyl)phenyl)-6,7- | J = 8.4 Hz, 2H), 7.49 (d, J = 8.4 Hz, 2H), 7.32 (d, | |
| | dihydro-2H- | J = 8.8 Hz, 2H), 6.41 (dd, J = 17.0, 10.0 Hz, | |
| | pyrazolo[3,4- | 1H), 6.27 (dd, J = 17.0, 2.0 Hz, 1H), 5.79 (dd, | |
| | d]pyridazin-2- | J = 10.0, 2.0 Hz, 1H), 4.85 (s, 2H), 3.46 (t, J = 6.6 | |
| | yl)phenyl)acrylamide | Hz, 2H), 3.36 (t, J = 5.3 Hz, 2H), 1.91-1.76 (m, 4H). | |
| 127 | 4-(2-(4- | ¹H NMR (400 MHz, DMSO) δ 11.38 (s, 1H), | 472 |
| | acrylamidophenyl)-4- | 10.35 (s, 1H), 9.22 (d, J = 6.4 Hz, 1H), 7.92 (d, | |
| | amino-7-oxo-6,7- | J = 8.4 Hz, 2H), 7.69 (d, J = 8.8 Hz, 2H), 7.56 (d, | |
| | dihydro-2H- | J = 8.4 Hz, 2H), 7.33 (d, J = 8.8 Hz, 2H), 6.46- | |
| | pyrazolo[3,4- | 6.37 (m, 1H), 6.27 (dd, J = 17.0, 2.0 Hz, 1H), | |
| | d]pyridazin-3-yl)-N- | 5.79 (dd, J = 10.0, 2.0 Hz, 1H), 5.03-4.93 (m, | |
| | (oxetan-3-yl)benzamide | 1H), 4.82 (s, 2H), 4.76 (t, J = 6.8 Hz, 2H), 4.58 | |
| | | (t, J = 6.4 Hz, 2H). | |
| 128 | N-(4-(4-amino-3-(3- | ¹H NMR (400 MHz, DMSO) δ 11.35 (s, 1H), | 528 |
| | methoxy-4-((6- | 10.49 (s, 1H), 7.80 (d, J = 8.9 Hz, 2H), 7.71- | |
| | methylpyridin-2- | 7.64 (m, 1H), 7.41 (d, J = 8.9 Hz, 2H), 7.24 (d, | |
| | yl)oxy)phenyl)-7-oxo- | J = 1.8 Hz, 1H), 7.18 (d, J = 8.1 Hz, 1H), 7.03 (dd, | |
| | 6,7-dihydro-2H- | J = 8.1, 1.9 Hz, 1H), 6.95 (d, J = 7.3 Hz, 1H), | |
| | pyrazolo[3,4- | 6.69 (d, J = 8.2 Hz, 1H), 5.74 (dd, J = 47.6, 3.7 | |
| | d]pyridazin-2- | Hz, 1H), 5.46 (dd, J = 15.6, 3.7 Hz, 1H), 4.95 (s, | |
| | yl)phenyl)-2- | 2H), 3.58 (s, 3H), 2.28 (s, 3H). | |
| | fluoroacrylamide | | |
| 129 | N-(4-(4-amino-3-(4- | ¹H NMR (400 MHz, DMSO) δ 11.30 (s, 1H), | 457 |
| | (cyclopentyloxy)phenyl)- | 10.34 (s, 1H), 7.69 (d, J = 8.9 Hz, 2H), 7.37- | |
| | 7-oxo-6,7-dihydro- | 7.26 (m, 4H), 6.97 (d, J = 8.7 Hz, 2H), 6.42 (dd, | |
| | 2H-pyrazolo[3,4- | J = 17.0, 10.1 Hz, 1H), 6.27 (dd, J = 17.0, 1.9 | |
| | d]pyridazin-2- | Hz, 1H), 5.79 (dd, J = 10.0, 1.9 Hz, 1H), 4.85 (t, | |
| | yl)phenyl)acrylamide | J = 5.7 Hz, 1H), 4.80 (s, 2H), 1.98-1.87 (m, | |
| | | 2H), 1.76-1.64 (m, 4H), 1.59 (d, J = 9.7 Hz, 2H). | |
| 130 | 4-(2-(4- | ¹H NMR (400 MHz, DMSO) δ 11.37 (s, 1H), | 485 |
| | acrylamidophenyl)-4- | 10.38 (s, 1H), 8.80 (s, 1H), 8.31 (s, 1H), 7.93 (d, | |
| | amino-7-oxo-6,7- | J = 8.2 Hz, 2H), 7.69 (d, J = 8.9 Hz, 2H), 7.55 | |
| | dihydro-2H- | (d, J = 8.3 Hz, 2H), 7.33 (d, J = 8.9 Hz, 2H), | |
| | pyrazolo[3,4- | 6.46-6.36 (m, 1H), 6.26 (dd, J = 17.0, 1.9 Hz, | |
| | d]pyridazin-3-yl)-N- | 1H), 5.78 (dd, J = 10.1, 1.9 Hz, 1H), 4.80 (s, | |
| | (pyrrolidin-3- | 2H), 4.51-4.41 (m, 1H), 3.24-3.10 (m, 2H), | |
| | yl)benzamide | 3.08-2.93 (m, 2H), 2.17-2.02 (m, 1H), 1.93- | |
| | | 1.81 (m, 1H). | |
| 131 | 4-(2-(4- | ¹H NMR (400 MHz, DMSO) δ 11.37 (s, 1H), | 500 |
| | acrylamidophenyl)-4- | 10.34 (s, 1H), 8.35 (d, J = 7.8 Hz, 1H), 7.89 (d, | |
| | amino-7-oxo-6,7- | J = 8.4 Hz, 2H), 7.68 (d, J = 8.9 Hz, 2H), 7.54 (d, | |
| | dihydro-2H- | J = 8.4 Hz, 2H), 7.33 (d, J = 8.9 Hz, 2H), 6.41 | |
| | pyrazolo[3,4- | (M, 1H), 6.27 (dd, J = 17.0, 2.0 Hz, 1H), 5.79 | |
| | d]pyridazin-3-yl)-N- | (dd, J = 10.0, 2.0 Hz, 1H), 4.80 (s, 2H), 3.96- | |
| | (tetrahydro-2H-pyran- | 3.83 (m, 1H), 3.82-3.71 (m, 2H), 3.29-3.23 | |
| | 3-yl)benzamide | (m, 1H), 3.19-3.09 (m, 1H), 1.96-1.86 (m, | |
| | | 1H), 1.72-1.64 (m, 1H), 1.61-1.52 (m, 2H). | |
| 132 | 4-(2-(4- | ¹H NMR (400 MHz, DMSO) δ 11.36 (s, 1H), | 500 |
| | acrylamidophenyl)-4- | 10.36 (s, 1H), 8.34 (d, J = 7.7 Hz, 1H), 7.71 (d, | |
| | amino-7-oxo-6,7- | J = 8.9 Hz, 2H), 7.61 (d, J = 7.8 Hz, 1H), 7.37 (d, | |
| | dihydro-2H- | J = 8.9 Hz, 2H), 7.23 (s, 1H), 7.05 (dd, J = 7.8, | |
| | pyrazolo[3,4- | 1.4 Hz, 1H), 6.42 (dd, J = 17.0, 10.1 Hz, 1H), | |
| | d]pyridazin-3-yl)-N- | 6.27 (dd, J = 17.0, 1.9 Hz, 1H), 5.79 (dd, J = | |
| | cyclobutyl-2- | 10.1, 1.9 Hz, 1H), 4.87 (s, 2H), 4.37 (dd, J = | |
| | methoxybenzamide | 16.5, 8.3 Hz, 1H), 3.76 (s, 3H), 2.20 (s, 2H), | |
| | | 1.99 (dd, J = 19.5, 7.9 Hz, 2H), 1.70-1.62 (m, 2H). | |
| 133 | N-(4-(4-amino-3-(3- | ¹H NMR (400 MHz, DMSO) δ 11.35 (s, 1H), | 525 |
| | methoxy-4-((4-methyl- | 10.00 (s, 1H), 8.42 (d, J = 5.0 Hz, 1H), 7.76 (d, | |
| | pyrimidin-2-yl)oxy) | J = 8.8 Hz, 2H), 7.38 (d, J = 8.8 Hz, 2H), 7.26 (dd, | |
| | phenyl)-7-oxo-6,7- | J = 4.7, 3.2 Hz, 2H), 7.12 (d, J = 5.0 Hz, 1H), | |
| | dihydro-2H-pyrazolo | 7.06 (dd, J = 8.1, 1.6 Hz, 1H), 5.81 (s, 1H), 5.55 | |
| | [3,4-d]pyridazin-2-yl) | (s, 1H), 4.92 (s, 2H), 3.58 (s, 3H), 2.38 (s, 3H), | |
| | phenyl)methacrylamide | 1.95 (s, 3H). | |
| 134 | N-(4-(4-amino-3-(3- | ¹H NMR (400 MHz, DMSO) δ 11.35 (s, 1H), | 511 |
| | methoxy-4-((4- | 10.36 (s, 1H), 8.42 (d, J = 5.0 Hz, 1H), 7.74 (d, | |
| | methylpyrimidin-2- | J = 8.9 Hz, 2H), 7.39 (d, J = 8.9 Hz, 2H), 7.26 (d, | |
| | yl)oxy)phenyl)-7-oxo- | J = 8.2 Hz, 2H), 7.12 (d, J = 5.0 Hz, 1H), 7.06 | |
| | 6,7-dihydro-2H- | (dd, J = 8.1, 1.9 Hz, 1H), 6.48-6.37 (m, 1H), | |
| | pyrazolo[3,4- | 6.28 (dd, J = 17.0, 1.9 Hz, 1H), 5.79 (dd, J = | |
| | d]pyridazin-2- | 10.0, 2.0 Hz, 1H), 4.92 (s, 2H), 3.57 (s, 3H), | |
| | yl)phenyl)acrylamide | 2.38 (s, 3H). | |
| 135 | 4-(2-(4- | ¹H NMR (400 MHz, DMSO) δ 11.38 (s, 1H), | 502 |
| | acrylamidophenyl)-4- | 10.37 (s, 1H), 8.20 (t, J = 5.9 Hz, 1H), 7.69 (dd, | |
| | amino-7-oxo-6,7- | J = 17.2, 8.4 Hz, 3H), 7.38 (d, J = 8.9 Hz, 2H), | |
| | dihydro-2H- | 7.25 (d, J = 1.2 Hz, 1H), 7.06 (dd, J = 7.8, 1.4 | |
| | pyrazolo[3,4- | Hz, 1H), 6.42 (dd, J = 17.0, 10.1 Hz, 1H), 6.28 | |
| | d]pyridazin-3-yl)-N- | (dd, J = 17.0, 1.9 Hz, 1H), 5.79 (dd, J = 10.0, 2.0 | |
| | isobutyl-2- | Hz, 1H), 4.89 (s, 2H), 3.78 (d, J = 11.8 Hz, 3H), | |
| | methoxybenzamide | 3.09 (t, J = 6.4 Hz, 2H), 1.81 (dt, J = 13.5, 6.7 | |
| | | Hz, 1H), 0.89 (t, J = 5.8 Hz, 6H). | |
| 136 | N-(4-(4-amino-3-(4-((4- | ¹H NMR (400 MHz, DMSO) δ 11.37 (s, 1H), | 530 |
| | chloropyridin-2- | 10.38 (s, 1H), 8.11 (d, J = 5.5 Hz, 1H), 7.73 (d, | |
| | yl)oxy)-3- | J = 8.9 Hz, 2H), 7.39 (d, J = 8.9 Hz, 2H), 7.29- | |
| | methoxyphenyl)-7-oxo- | 7.20 (m, 3H), 7.17 (d, J = 1.6 Hz, 1H), 7.06 (dd, | |
| | 6,7-dihydro-2H- | J = 8.1, 1.9 Hz, 1H), 6.43 (dd, J = 17.0, 10.1 Hz, | |
| | pyrazolo[3,4- | 1H), 6.29 (dd, J = 17.0, 2.0 Hz, 1H), 5.80 (dd, | |
| | d]pyridazin-2- | J = 10.0, 2.0 Hz, 1H), 4.96 (s, 2H), 3.59 (s, 3H). | |
| | yl)phenyl)acrylamide | | |
| 137 | 4-(4-amino-2-(4-(2- | ¹H NMR (400 MHz, DMSO) δ 11.37 (s, 1H), | 518 |
| | fluoroacrylamido)phe- | 10.48 (s, 1H), 8.34 (d, J = 7.6 Hz, 1H), 7.78 (d, | |
| | nyl)-7-oxo-6,7-dihydro- | J = 8.8 Hz, 2H), 7.61 (d, J = 7.8 Hz, 1H), 7.40 (d, | |
| | 2H-pyrazolo[3,4- | J = 8.8 Hz, 2H), 7.24 (s, 1H), 7.05 (d, J = 7.8 | |
| | d]pyridazin-3-yl)-N- | Hz, 1H), 5.73 (dd, J = 47.6, 3.6 Hz, 1H), 5.46 | |
| | cyclobutyl-2- | (dd, J = 15.5, 3.6 Hz, 1H), 4.88 (s, 2H), 4.38 (d, | |
| | methoxybenzamide | J = 8.1 Hz, 1H), 3.76 (s, 3H), 2.20 (s, 2H), 2.06- | |
| | | 1.93 (m, 2H), 1.66 (dd, J = 12.1, 5.6 Hz, 2H). | |
| 138 | N-(4-(4-amino-3-(4- | ¹H NMR (400 MHz, DMSO) δ 11.30 (s, 1H), | 487 |
| | (cyclopentyloxy)-3- | 10.35 (s, 1H), 7.70 (d, J = 8.9 Hz, 2H), 7.35 (d, | |
| | methoxyphenyl)-7-oxo- | J = 8.9 Hz, 2H), 7.04-6.97 (m, 2H), 6.92 (dd, J = | |
| | 6,7-dihydro-2H- | 8.2, 1.9 Hz, 1H), 6.43 (dd, J = 17.0, 10.1 Hz, | |
| | pyrazolo[3,4- | 1H), 6.27 (dd, J = 17.0, 2.0 Hz, 1H), 5.79 (dd, | |
| | d]pyridazin-2- | J = 10.0, 2.0 Hz, 1H), 4.87 (s, 2H), 4.81 (t, J = 5.7 | |
| | yl)phenyl)acrylamide | Hz, 1H), 3.62 (d, J = 5.6 Hz, 3H), 1.91 (dd, J = | |
| | | 11.9, 7.2 Hz, 2H), 1.69 (dd, J = 9.3, 5.1 Hz, 4H), | |
| | | 1.62-1.48 (m, 2H). | |
| 139 | N-(4-(4-amino-3-(4- | ¹H NMR (400 MHz, DMSO) δ 11.32 (s, 1H), | 443 |
| | cyclobutoxyphenyl)-7- | 10.37 (s, 1H), 7.69 (d, J = 8.9 Hz, 2H), 7.33 (dd, | |
| | oxo-6,7-dihydro-2H- | J = 8.7, 7.6 Hz, 4H), 6.92 (d, J = 8.7 Hz, 2H), | |
| | pyrazolo[3,4- | 6.42 (dd, J = 17.0, 10.1 Hz, 1H), 6.28 (dd, J = | |
| | d]pyridazin-2- | 17.0, 2.0 Hz, 1H), 5.78 (dt, J = 13.0, 6.5 Hz, | |
| | yl)phenyl)acrylamide | 1H), 4.77 (d, J = 16.7 Hz, 2H), 4.70 (dd, J = | |
| | | 14.2, 7.1 Hz, 1H), 2.42 (ddd, J = 11.7, 9.3, 4.6 | |
| | | Hz, 2H), 2.10-1.99 (m, 2H), 1.78 (q, J = 10.3 | |
| | | Hz, 1H), 1.64 (tt, J = 10.1, 5.1 Hz, 1H). | |
| 140 | N-(4-(4-amino-3-(4- | ¹H NMR (400 MHz, DMSO) δ 11.32 (s, 1H), | 471 |
| | (cyclohexyloxy)phenyl)- | 10.38 (s, 1H), 7.69 (d, J = 8.8 Hz, 2H), 7.32 (d, | |
| | 7-oxo-6,7-dihydro-2H- | J = 8.8 Hz, 4H), 7.01 (d, J = 8.8 Hz, 2H), 6.50- | |
| | pyrazolo[3,4- | 6.37 (m, 1H), 6.28 (dd, J = 17.0, 2.0 Hz, 1H), | |
| | d]pyridazin-2- | 5.79 (dd, J = 10.0, 2.0 Hz, 1H), 4.81 (s, 2H), | |
| | yl)phenyl)acrylamide | 4.44-4.33 (m, 1H), 1.99-1.89 (m, 2H), 1.76- | |
| | | 1.64 (m, 2H), 1.58-1.49 (m, 1H), 1.44-1.34 | |
| | | (m, 4H), 1.29-1.24 (m, 1H). | |
| 141 | N-(4-(4-amino-3-(4- | ¹H NMR (400 MHz, DMSO) δ 11.31 (d, J = 14.0 | 445 |
| | isobutoxyphenyl)-7- | Hz, 1H), 10.39 (d, J = 17.6 Hz, 1H), 7.68 (t, J = | |
| | oxo-6,7-dihydro-2H- | 9.4 Hz, 2H), 7.34 (t, J = 8.1 Hz, 4H), 7.01 (d, J = | |
| | pyrazolo[3,4- | 8.5 Hz, 2H), 6.42 (dd, J = 16.9, 10.0 Hz, 1H), | |
| | d]pyridazin-2- | 6.28 (d, J = 17.0 Hz, 1H), 5.79 (d, J = 10.0 Hz, | |
| | yl)phenyl)acrylamide | 1H), 4.80 (s, 2H), 3.77 (d, J = 6.4 Hz, 2H), 2.00 | |
| | | (td, J = 13.2, 6.5 Hz, 1H), 0.96 (t, J = 8.1 Hz, 6H). | |
| 142 | N-(4-(4-amino-3-(4- | ¹H NMR (400 MHz, DMSO) δ 11.33 (s, 1H), | 475 |
| | (cyclopentyloxy)-3- | 10.36 (s, 1H), 7.70 (d, J = 8.9 Hz, 2H), 7.34 (d, | |
| | fluorophenyl)-7-oxo- | J = 8.9 Hz, 2H), 7.19 (dd, J = 8.7, 5.0 Hz, 2H), | |
| | 6,7-dihydro-2H- | 6.43 (dd, J = 17.0, 10.0 Hz, 1H), 6.28 (dd, J = | |
| | pyrazolo[3,4- | 17.0, 2.0 Hz, 1H), 5.79 (dd, J = 10.1, 2.0 Hz, | |
| | d]pyridazin-2- | 1H), 5.33 (t, J = 4.6 Hz, 1H), 4.92 (s, 1H), 4.85 | |
| | yl)phenyl)acrylamide | (s, 2H), 1.73 (d, J = 11.6 Hz, 4H), 1.59 (s, 2H), | |
| | | 1.46 (s, 2H). | |
| 143 | 5-(4- | ¹H NMR (400 MHz, DMSO) δ 13.29 (s, 1H), | 520 |
| | acrylamidophenyl)-6- | 10.11 (s, 1H), 8.17 (s, 1H), 7.78 (s, 1H), 7.64 | |
| | (3-methoxy-4-((6- | (dd, J = 15.9, 8.1 Hz, 2H), 7.59-7.49 (m, 3H), | |
| | methylpyridin-2- | 7.02 (d, J = 8.5 Hz, 2H), 6.93 (dd, J = 15.8, 7.7 | |
| | yl)oxy)phenyl)-1H- | Hz, 2H), 6.82-6.74 (m, 2H), 6.46-6.37 (m, | |
| | indazole-7-carboxamide | 2H), 6.25 (dd, J = 17.0, 1.9 Hz, 1H), 5.75 (dd, | |
| | | J = 10.1, 1.9 Hz, 1H), 3.43 (s, 3H), 2.31 (s, 3H). | |
| 144 | N-(4-(4-amino-7-oxo-3- | ¹H NMR (400 MHz, DMSO) δ 11.32 (s, 1H), | 473 |
| | (4-((tetrahydro-2H- | 10.36 (s, 1H), 7.69 (d, J = 8.9 Hz, 2H), 7.33 (t, | |
| | pyran-4- | J = 8.3 Hz, 4H), 7.06 (d, J = 8.8 Hz, 2H), 6.42 (dd, | |
| | yl)oxy)phenyl)-6,7- | J = 17.0, 10.0 Hz, 1H), 6.27 (dd, J = 17.0, 1.9 | |
| | dihydro-2H- | Hz, 1H), 5.79 (dd, J = 10.0, 1.9 Hz, 1H), 4.81 (s, | |
| | pyrazolo[3,4- | 2H), 4.68-4.56 (m, 1H), 3.91-3.79 (m, 2H), | |
| | d]pyridazin-2- | 3.47 (m, 2H), 1.97 (d, J = 10.1 Hz, 2H), 1.59 | |
| | yl)phenyl)acrylamide | (ddd, J = 18.7, 11.7, 6.8 Hz, 2H). | |
| 145 | N-(4-(4-amino-3-(6- | ¹H NMR (400 MHz, DMSO) δ 11.35 (s, 1H), | 472 |
| | (cyclohexyloxy)pyridin- | 10.37 (s, 1H), 8.18 (d, J = 2.8 Hz, 1H), 7.71 (d, | |
| | 3-yl)-7-oxo-6,7- | J = 8.8 Hz, 2H), 7.68 (dd, J = 8.6, 2.4 Hz, 1H), | |
| | dihydro-2H- | 7.35 (d, J = 8.8 Hz, 2H), 6.80 (d, J = 8.6 Hz, | |
| | pyrazolo[3,4- | 1H), 6.42 (dd, J = 17.0, 10.0 Hz, 1H), 6.28 (dd, | |
| | d]pyridazin-2- | J = 17.0, 2.0 Hz, 1H), 5.79 (dd, J = 10.0, 2.0 Hz, | |
| | yl)phenyl)acrylamide | 1H), 5.03-4.95 (m, 1H), 4.89 (s, 2H), 1.99- | |
| | | 1.92 (m, 2H), 1.75-1.67 (m, 2H), 1.58-1.51 | |
| | | (m, 1H), 1.48-1.34 (m, 4H), 1.34-1.29 (m, 1H). | |
| 146 | N-(4-(4-amino-3-(4- | ¹H NMR (400 MHz, DMSO) δ 11.26 (s, 1H), | 484 |
| | (cyclohexyl(methyl)ami- | 10.42 (s, 1H), 7.71 (d, J = 8.9 Hz, 2H), 7.33 (d, | |
| | no)phenyl)-7-oxo-6,7- | J = 8.9 Hz, 2H), 7.17 (d, J = 8.8 Hz, 2H), 6.79 (d, | |
| | dihydro-2H- | J = 8.9 Hz, 2H), 6.44 (dd, J = 17.0, 10.1 Hz, | |
| | pyrazolo[3,4- | 1H), 6.27 (dd, J = 17.0, 1.9 Hz, 1H), 5.78 (dd, | |
| | d]pyridazin-2- | J = 10.0, 1.9 Hz, 1H), 4.84 (s, 2H), 3.65 (t, J = | |
| | yl)phenyl)acrylamide | 11.2 Hz, 1H), 2.76 (s, 3H), 1.76 (d, J = 12.3 Hz, | |
| | | 2H), 1.63 (t, J = 12.0 Hz, 3H), 1.55-1.29 (m, 5H). | |
| 147 | N-(4-(4-amino-3-(4- | ¹H NMR (400 MHz, DMSO) δ 11.30 (s, 1H), | 507 |
| | ((4,4-difluoro- | 10.34 (s, 1H), 7.69 (d, J = 8.8 Hz, 2H), 7.34 (dd, | |
| | cyclohexyl)oxy)phenyl)- | J = 13.6, 8.7 Hz, 4H), 7.08 (d, J = 8.6 Hz, 2H), | |
| | 7-oxo-6,7-dihydro-2H- | 6.46-6.39 (m, 1H), 6.29-6.25 (m, 1H), 5.80- | |
| | pyrazolo[3,4-d] | 5.77 (m, 1H), 4.80 (s, 2H), 4.69-4.59 (m, 1H), | |
| | pyridazin-2-yl)phenyl) | 2.10-1.90 (m, 6H), 1.82-1.77 (m, 2H). | |
| | acrylamide | | |
| 148 | N-(4-(4-amino-3-(4- | ¹H NMR (400 MHz, DMSO-d6) δ 11.33 (s, 1H), | 487 |
| | (cyclohexylthio)phenyl)- | 10.34 (s, 1H), 7.69 (d, J = 8.8 Hz, 2H), 7.40-7.34 | |
| | 7-oxo-6,7-dihydro-2H- | (m, 4H), 7.31 (d, J = 8.8 Hz, 2H), 6.47-6.35 (m, | |
| | pyrazolo[3,4- | 1H), 6.27 (dd, J = 16.8, 10.0 Hz, 1H), 5.78 (dd, | |
| | d]pyridazin-2- | J = 10.0, 2.0 Hz, 1H), 4.81 (s, 2H), 3.42-3.38 (m, | |
| | yl)phenyl)acrylamide | 1H), 1.96-1.89 (m, 2H), 1.74-1.66 (m, 2H), 1.62- | |
| | | 1.50 (m, 2H), 1.37-1.32 (m, 2H), 1.26-1.20 (s, | |
| | | 2H). | |
| 149 | N-(4-(4-amino-3-(4- | ¹H NMR (400 MHz, DMSO-d6) difluoro 11.30 (s, 1H), | 471 |
| | (cyclopentyloxy)phenyl)- | 9.97 (s, 1H), 7.73 (d, J = 8.0 Hz, 2H), 7.35 (t, | |
| | 7-oxo-6,7-dihydro- | J = 8.0 Hz, 4H), 6.99 (d, J = 8.0 Hz, 2H), 5.81 (s, | |
| | 2H-pyrazolo[3,4-d] | 1H), 5.55 (s, 1H), 4.87 (t, J = 8.0 Hz, 1H), 4.81 | |
| | pyridazin-2-yl)phenyl) | (s, 2H), 1.96-1.90 (m, 5H), 1.74-1.68 (m, 4H), | |
| | methacrylamide | 1.61-1.56 (m, 2H). | |
| 150 | 4-(2-(4-acrylamido-2- | ¹H NMR (400 MHz, MeOD) δ 8.14 (s, 1H), 7.89- | 567 |
| | fluorophenyl)-4-amino- | 7.84 (m, 1H), 7.82-7.74 (m, 1H), 7.38-7.28 | |
| | 7-cyano-1-methyl-1H- | (m, 1H), 7.26-7.15 (m, 1H), 7.10-6.89 (m, | |
| | pyrrolo[3,2-c]pyridin-3- | 2H), 6.44-6.37 (m, 2H), 5.81 (m, 1H), 4.14- | |
| | yl)-2-methoxy-N- | 4.07 (m, 2H), 3.88 (s, 3H), 3.82 (s, 3H). | |
| | (2,2,2-trifluoroethyl) | | |
| | benzamide | | |
| 151 | 4-(4-amino-7-cyano-2- | ¹H NMR (400 MHz, MeOD) δ 8.15 (s, 1H), 7.95- | 585 |
| | (2-fluoro-4-(2- | 7.83 (m, 1H), 7.82-7.67 (m, 1H), 7.50-7.40 | |
| | fluoroacrylamido)phenyl)- | (m, 1H), 7.29-7.18 (m, 1H), 7.12-6.84 (m, | |
| | 1-methyl-1H-pyrrolo | 2H), 5.82-5.66 (m, 1H), 5.42-5.24 (m, 1H), | |
| | [3,2-c]pyridin-3-yl)-2- | 4.11 (q, J = 9.3 Hz, 2H), 3.88 (s, 3H), 3.82 (s, | |
| | methoxy-N-(2,2,2- | 3H). | |
| | trifluoroethyl)benzamide | | |
| 152 | 4-(4-amino-6-(4-(2- | ¹H NMR (400 MHz, DMSO) δ 10.36 (s, 1H), | 518 |
| | fluoroacrylamido)-2- | 8.16 (s, 1H), 8.05 (d, J = 4.0 Hz, 1H), 7.65 (d, | |
| | methoxyphenyl)pyrazolo[5,1- | J = 7.6 Hz, 1H), 7.44-7.41 (m, 2H), 7.29 (d, J = | |
| | f][1,2,4]triazin-5- | 8.0 Hz, 1H), 6.95 (s, 1H), 6.86 (d, J = 8.0 Hz, | |
| | yl)-N-cyclopropyl-2- | 1H), 5.78-5.66 (m, 1H), 5.48-5.43 (m, 1H), | |
| | methoxybenzamide | 3.70 (s, 3H), 3.36 (s, 3H), 2.81 (m, 1H), 0.72- | |
| | | 0.63 (m, 2H), 0.56-0.48 (m, 2H). | |
| 153 | 4-(4-amino-6-(2-fluoro- | ¹H NMR (400 MHz, DMSO) δ 10.56 (s, 1H), | 506 |
| | 4-(2-fluoroacrylamido) | 8.20 (s, 1H), 8.09 (d, J = 4.4 Hz, 1H), 7.70-7.56 | |
| | phenyl)pyrazolo[5,1- | (m, 3H), 7.44 (t, J = 8.4 Hz, 1H), 7.04 (s, 1H), | |
| | f][1,2,4]triazin-5-yl)-N- | 6.90 (d, J = 8.0 Hz, 1H), 5.81-5.68 (m, 1H), | |
| | cyclopropyl-2- | 5.51-5.46 (m, 1H), 3.73 (s, 3H), 2.83 (m, 1H), | |
| | methoxybenzamide | 0.73-0.65 (m, 2H), 0.58-0.49 (m, 2H). | |
| 154 | 4-(6-(4-acrylamido-2- | ¹H NMR (400 MHz, DMSO) δ 10.26 (s, 1H), | 500 |
| | methoxyphenyl)-4- | 8.16 (s, 1H), 8.04 (d, J = 4.4 Hz, 1H), 7.66 (d, | |
| | aminopyrazolo[5,1- | J = 8.0 Hz, 1H), 7.43 (s, 1H), 7.30-7.23 (m, 2H), | |
| | f][1,2,4]triazin-5-yl)-N- | 6.95 (s, 1H), 6.87 (d, J = 8.0 Hz, 1H), 6.47-6.40 | |
| | cyclopropyl-2- | (m, 1H), 6.26 (m, 1H), 5.78 (m, 1H), 3.70 (s, | |
| | methoxybenzamide | 3H), 3.36 (s, 3H), 2.82 (m, 1H), 0.70-0.66 (m, | |
| | | 2H), 0.56-0.48 (m, 2H). | |
| 155 | 4-(6-(4-acrylamido-2- | ¹H NMR (400 MHz, DMSO) difluoro 10.44 (s, 1H), | 488 |
| | fluorophenyl)-4- | 8.20 (s, 1H), 8.08 (d, J = 4.4 Hz, 1H), 7.69-7.60 | |
| | aminopyrazolo[5,1-f] | (m, 2H), 7.41 (m, 2H), 7.04 (s, 1H), 6.90 (d, J = | |
| | [1,2,4]triazin-5-yl)-N- | 8.0 Hz, 1H), 6.42 (m, 1H), 6.29 (m, 1H), 5.81 | |
| | cyclopropyl-2- | (m, 1H), 3.73 (s, 3H), 2.83 (m, 1H), 0.73-0.65 | |
| | methoxybenzamide | (m, 2H), 0.58-0.49 (m, 2H). | |
| 156 | 4-(2-(4-acrylamido-2- | ¹H NMR (400 MHz, DMSO) δ 10.31 (s, 1H), | 537 |
| | methoxyphenyl)-4- | 8.19 (s, 1H), 8.04 (d, J = 4.4 Hz, 1H), 7.59-7.57 | |
| | amino-7-cyano-1- | (m, 2H), 7.18-7.16 (m, 1H), 7.04 (d, J = 8.0 Hz, | |
| | methyl-1H-pyrrolo[3,2- | 1H), 6.85 (s, 2H), 6.46-6.40 (m, 1H), 6.30-6.25 | |
| | c]pyridin-3-yl)-N- | (m, 1H), 5.80-5.77 (m, 1H), 3.74 (s, 3H), 3.67 (s, | |
| | cyclopropyl-2- | 6H), 2.84-2.77 (m, 1H), 0.69-0.63 (m, 2H), | |
| | methoxybenzamide | 0.53-0.49 (m, 2H). | |
| 157 | 4-(2-(4-acrylamido-2- | ¹H NMR (400 MHz, DMSO) δ 10.53 (s, 1H), | 525 |
| | fluorophenyl)-4-amino- | 8.23 (s, 1H), 8.06 (d, J = 4.2 Hz, 1H), 7.78 (d, | |
| | 7-cyano-1-methyl-1H- | J = 11.1 Hz, 1H), 7.58 (d, J = 7.8 Hz, 1H), 7.33 | |
| | pyrrolo[3,2-c]pyridin-3- | (dd, 2H), 6.92-6.83 (m, 2H), 6.61-5.88 (m, | |
| | yl)-N-cyclopropyl-2- | 4H), 5.82 (dd, J = 10.1, 1.7 Hz, 1H), 3.75 (s, | |
| | methoxybenzamide | 3H), 3.70 (s, 3H), 2.84-2.76 (m, 1H), 0.69- | |
| | | 0.64 (m, 2H), 0.54-0.49 (m, 2H). | |
| 158 | 4-(4-amino-7-cyano-2- | ¹H NMR (400 MHz, DMSO) δ 10.41 (s, 1H), | 527 |
| | (4-(2-fluoroacrylamido) | 8.21 (s, 1H), 7.89 (d, J = 7.7 Hz, 1H), 7.76 (d, | |
| | phenyl)-1-methyl-1H- | J = 8.5 Hz, 2H), 7.62 (d, J = 7.8 Hz, 1H), 7.33 (d, | |
| | pyrrolo[3,2-c]pyridin-3- | J = 8.5 Hz, 2H), 6.94 (s, 1H), 6.87 (d, J = 7.9 Hz, | |
| | yl)-N-isopropyl-2- | 1H), 5.78 (d, J = 3.6 Hz, 0.5H), 5.66 (d, J = 3.7 | |
| | methoxybenzamide | Hz, 0.5H), 5.45 (dd, J = 15.6, 3.6 Hz, 1H), 4.09- | |
| | | 3.99 (m, 1H), 3.80 (s, 3H), 3.72 (s, 3H) 1.14 (d, | |
| | | J = 6.5 Hz, 6H). | |
| 159 | (S)-4-(4-amino-6-(4-(2- | ¹H NMR (400 MHz, MeOD) difluoro 8.10 (s, 1H), 7.93 | 552 |
| | fluoroacrylamido)phe- | (d, J = 7.8 Hz, 1H), 7.63 (d, J = 8.6 Hz, 2H), | |
| | nyl)pyrazolo[5,1-f][1,2,4] | 7.48 (d, J = 8.6 Hz, 2H), 7.21-7.12 (m, 2H), | |
| | triazin-5-yl)-N-(3,3- | 5.76 (d, J = 3.4 Hz, 0.5H), 5.65 (d, J = 3.4 Hz, | |
| | difluorocyclopentyl)-2- | 0.5H), 5.29 (dd, J = 15.2, 3.5 Hz, 1H), 4.60- | |
| | methoxybenzamide | 4.51 (m, 1H), 3.86 (s, 3H), 2.65-2.50 (m, 1H), | |
| | | 2.37-2.07 (m, 4H), 1.89 (dd, J = 11.8, 7.4 Hz, 1H). | |
| 160 | 4-(2-(4-acrylamido- | ¹H NMR (400 MHz, DMSO) δ 10.29 (s, 1H), | 537 |
| | phenyl)-4-amino-7- | 8.95 (m, 1H), 8.22 (s, 1H), 7.71 (d, J = 8.6 Hz, | |
| | cyano-1-methyl-1H- | 2H), 7.59 (m, 1H), 7.28 (d, J = 8.6 Hz, 2H), 7.19- | |
| | pyrrolo[3,2-c]pyridin-3- | 7.09 (m, 2H), 6.43 (m, 1H), 6.27 (m, 1H), 5.78 | |
| | yl)-2-fluoro-N-(2,2,2- | (m, 1H), 4.06 (m, 2H), 3.79 (s, 3H). | |
| | trifluoroethyl) | | |
| | benzamide | | |
| 161 | N-(4-(4-amino-7- | ¹H NMR (400 MHz, DMSO) δ 10.26 (s, 1H), | 478 |
| | cyano-3-(4- | 8.16 (d, J = 2.4 Hz, 1H), 7.67 (d, J = 8.8 Hz, | |
| | (cyclopentyloxy)phenyl)- | 2H), 7.26 (d, J = 8.4 Hz, 2H), 7.14 (d, J = 8.4 | |
| | 1-methyl-1H- | Hz, 2H), 6.88 (d, J = 8.8 Hz, 2H), 6.43 (m, 1H), | |
| | pyrrolo[3,2-c]pyridin-2- | 6.26 (m, 1H), 5.77 (m, 1H), 4.79 (s, 1H), 3.79 (s, | |
| | yl)phenyl)acrylamide | 3H), 1.90 (m, 2H), 1.69 (m, 4H), 1.56 (m, 2H). | |
| 162 | 4-(6-(4- | ¹H NMR (400 MHz, DMSO) δ 10.22 (s,1H), | 484 |
| | acrylamidophenyl)-4- | 8.32 (s, 1H), 8.16 (s, 1H), 7.78 (d, J = 7.8 Hz, | |
| | aminopyrazolo[5,1-f] | 1H), 7.62 (d, J = 8.6 Hz, 2H), 7.40 (d, J = 8.5 | |
| | [1,2,4]triazin-5-yl)-2- | Hz, 2H), 7.18 (s, 1H), 7.01 (d, J = 7.8 Hz, 1H), | |
| | methoxy-N-(1- | 6.43 (dd, J = 17.0, 10.1 Hz, 1H), 6.25 (d, J = | |
| | methylcyclopropyl)benzamide | 15.7 Hz, 1H), 5.76 (d, J = 11.5 Hz, 1H), 3.81 (s, | |
| | | 3H), 1.40 (s, 3H), 0.75 (t, J = 5.4 Hz, 2H), 0.63 | |
| | | (t, J = 5.7 Hz, 2H). | |
| 163 | 4-(4-amino-6-(4-(2- | ¹H NMR (400 MHz, DMSO) δ 10.36 (s, 1H), | 502 |
| | fluoroacrylamido)phe- | 8.74-8.20 (m, 1H), 8.17 (s, 1H), 7.86-7.82 (m, | |
| | nyl)pyrazolo[5,1-f] | 1H), 7.71-7.65 (m, 2H), 7.45-7.40 (m, 2H), | |
| | [1,2,4]triazin-5-yl)-N- | 7.22 (s, 1H), 7.06-7.01 (m, 1H), 5.98-5.51 (m, | |
| | (cyclopropylmethyl)-2- | 2H), 5.47-5.40 (m, 1H), 3.84 (s, 3H), 3.21 (t, | |
| | methoxybenzamide | J = 6.2 Hz, 2H), 1.10-1.02 (m, 1H), 0.48-0.42 | |
| | | (m, 2H), 0.29-0.23 (m, 2H). | |
| 164 | N-(4-(4-amino-5-(4- | ¹H NMR (400 MHz, DMSO) δ 10.20 (s, 1H), | 454 |
| | (cyclopentyl(methyl)ami- | 8.10 (s, 1H), 7.61 (d, J = 8.7 Hz, 2H), 7.46 (d, | |
| | no)phenyl)pyrazolo[5,1- | J = 8.8 Hz, 2H), 7.19 (d, J = 8.8 Hz, 2H), 6.91 (d, | |
| | f][1,2,4]triazin-6- | J = 8.8 Hz, 2H), 6.42 (dd, J = 17.0, 10.2 Hz, 1H), | |
| | yl)phenyl)acrylamide | 6.32-6.14 (m, 1H), 5.76 (dd, J = 10.1, 2.0 Hz, | |
| | | 1H), 4.40-4.23 (m, 1H), 2.80 (s, 3H), 1.86 (t, | |
| | | J = 9.6 Hz, 2H), 1.70 (t, J = 10.4 Hz, 2H), 1.65- | |
| | | 1.50 (m, 4H). | |
| 165 | N-(4-(4-amino-5-(4- | ¹H NMR (400 MHz, DMSO) δ10.21 (s, 1H), | 427 |
| | cyclobutoxyphenyl)pyrazolo[5,1- | 8.12 (s, 1H), 7.60 (d, J = 8.7 Hz, 2H), 7.35 (dd, | |
| | f][1,2,4] | J = 28.1, 8.7 Hz, 4H), 6.98 (d, J = 8.6 Hz, 2H), | |
| | triazin-6-yl)phenyl) | 6.42 (dd, J = 17.0, 10.1 Hz, 1H), 6.25 (dd, J = | |
| | acrylamide | 17.0, 1.9 Hz, 1H), 5.76 (dd, J = 10.1, 2.0 Hz, | |
| | | 1H), 4.82-4.68 (m, 1H), 2.46-2.43 (m, 2H), | |
| | | 2.18-2.04 (m, 2H), 1.77-1.67 (m, 2H). | |
| 166 | 4-(4-amino-6-(6- | ¹H NMR (400 MHz, DMSO) δ 8.74 (t, J = 6.4 | 468 |
| | ethynylpyridin-3-yl) | Hz, 1H), 8.58 (d, J = 1.8 Hz, 1H), 8.21 (s, 1H), | |
| | pyrazolo[5,1-f][1,2,4] | 7.86 (d, J = 7.8 Hz, 1H), 7.80 (dd, J = 8.2, 2.2 | |
| | triazin-5-yl)-2-methoxy- | Hz, 1H), 7.57 (d, J = 8.2 Hz, 1H), 7.29 (s, 1H), | |
| | N-(2,2,2-trifluoroethyl) | 7.06 (d, J = 8.0 Hz, 1H), 4.41 (s, 1H), 4.20-4.09 | |
| | benzamide | (m, 2H), 3.85 (s, 3H). | |
| 167 | 4-(2-(4- | ¹H NMR (400 MHz, DMSO) δ 10.28 (s, 1H), | 543 |
| | acrylamidophenyl)-4- | 8.37 (s, 1H), 8.20 (d, J = 6.6 Hz, 1H), 7.69 (d, | |
| | amino-7-cyano-1- | J = 8.5 Hz, 2H), 7.64 (d, J = 7.8 Hz, 1H), 7.30 (d, | |
| | methyl-1H-pyrrolo[3,2- | J = 8.4 Hz, 2H), 6.96 (s, 1H), 6.90 (d, J = 8.0 Hz, | |
| | c]pyridin-3-yl)-N-(2,2- | 1H), 6.46-6.40 (m, 1H), 6.34-5.85 (m, 1H), | |
| | difluorocyclopropyl)-2- | 5.81-5.76 (m, 1H), 3.80 (s, 3H), 3.71 (s, 3H), | |
| | methoxybenzamide | 3.49-3.40 (m, 1H), 1.98-1.86 (m, 1H), 1.73- | |
| | | 1.62 (m, 1H). | |
| 168 | 4-(2-(4- | ¹H NMR (400 MHz, DMSO) δ 10.28 (s, 1H), | 539 |
| | acrylamidophenyl)-4- | 8.36 (d, J = 6.9 Hz, 1H), 8.21 (s, 1H), 7.69 (d, | |
| | amino-7-cyano-1- | J = 8.5 Hz, 2H), 7.57 (d, J = 7.8 Hz, 1H), 7.30 (d, | |
| | methyl-1H-pyrrolo[3,2- | J = 8.4 Hz, 2H), 6.95-6.86 (m, 2H), 6.43 (dd, | |
| | c]pyridin-3-yl)-N-(3- | J = 17.0, 10.1 Hz, 1H), 6.27 (d, J = 15.2 Hz, 1H), | |
| | fluorocyclobutyl)-2- | 5.78 (d, J = 12.0 Hz, 1H), 5.24 (d, J = 56.7 Hz, | |
| | methoxybenzamide | 1H), 4.50 (m, 1H), 3.80 (s, 3H), 3.71 (s, 3H), | |
| | | 2.33 (m, 2H), 1.16 (m, 2H). | |
| 169 | 4-(2-(4-acryl- | ¹H NMR (400 MHz, DMSO) δ 10.27 (s, 1H), | 521 |
| | amidophenyl)-4-amino- | 8.21 (d, J = 6.7 Hz, 2H), 7.69 (d, J = 8.4 Hz, | |
| | 7-cyano-1-methyl-1H- | 2H), 7.63 (d, J = 7.7 Hz, 1H), 7.29 (d, J = 8.5 | |
| | pyrrolo[3,2-c]pyridin-3- | Hz, 2H), 6.93-6.83 (m, 2H), 6.43 (dd, J = 17.0, | |
| | yl)-2-methoxy-N-(1- | 10.0 Hz, 1H), 6.26 (d, J = 17.2 Hz, 1H), 5.78 (d, | |
| | methylcyclopropyl)benzamide | J = 11.5 Hz, 1H), 3.80 (s, 3H), 3.70 (s, 3H), 1.35 | |
| | | (s, 3H), 0.70 (s, 2H), 0.58 (t, J = 5.6 Hz, 2H). | |
| 170 | 4-(2-(4-acrylamido- | ¹H NMR (400 MHz, DMSO) δ 10.28 (s, 1H), | 521 |
| | phenyl)-4-amino-7- | 8.26-8.16 (m, 2H), 7.73-7.65 (m, 3H), 7.33- | |
| | cyano-1-methyl-1H- | 7.26 (m, 2H), 6.97-6.92 (m, 1H), 6.91-6.83 | |
| | pyrrolo[3,2-c]pyridin-3- | (m, 1H), 6.58-5.63 (m, 5H), 3.81 (s, 3H), 3.73 | |
| | yl)-N- | (s, 3H), 3.18-3.12 (m, 2H), 1.06-0.97 (m, 1H), | |
| | (cyclopropylmethyl)-2- | 0.45-0.37 (m, 2H), 0.26-0.19 (m, 2H). | |
| | methoxybenzamide | | |
| 171 | 4-(4-amino-7-cyano-2- | ¹H NMR (400 MHz, DMSO) δ 10.41 (s, 1H), | 555 |
| | (4-(2- | 8.20 (s, 1H), 8.05 (d, J = 4.2 Hz, 1H), 7.65-7.52 | |
| | fluoroacrylamido)-2- | (m, 2H), 7.34 (d, J = 8.2 Hz, 1H), 7.08 (d, J = 8.2 | |
| | methoxyphenyl)-1- | Hz, 1H), 6.85 (s, 2H), 6.09 (s, 2H), 5.80-5.67 (m, | |
| | methyl-1H-pyrrolo[3,2- | 1H), 5.49-5.44 (m, 1H), 3.73 (s, 3H), 3.68 (s, | |
| | c]pyridin-3-yl)-N- | 6H), 2.82-2.77 (m, 1H), 0.74-0.61 (m, 2H), 0.53- | |
| | cyclopropyl-2- | 0.50 (m, 2H). | |
| | methoxybenzamide | | |
| 172 | N-(4-(4-amino-3-(4- | ¹H NMR (400 MHz, DMSO) δ 11.31 (s, 1H), | 429 |
| | cyclopropoxyphenyl)-7- | 10.34 (s, 1H), 7.69 (d, J = 8.9 Hz, 2H), 7.35 (dd, | |
| | oxo-6,7-dihydro-2H- | J = 20.5, 8.8 Hz, 4H), 7.14 (d, J = 8.8 Hz, 2H), | |
| | pyrazolo[3,4- | 6.42 (dd, J = 16.9, 10.1 Hz, 1H), 6.27 (dd, J = | |
| | d]pyridazin-2- | 17.0, 2.0 Hz, 1H), 5.82-5.73 (m, 1H), 4.79 (s, | |
| | yl)phenyl)acrylamide | 2H), 3.94-3.85 (m, 1H), 0.84-0.75 (m, 2H), | |
| | | 0.73-0.56 (m, 2H). | |
| 173 | N-(4-(4-amino-3-(4-((3- | ¹H NMR (400 MHz, DMSO) δ 11.30 (s, 1H), | 475 |
| | fluorocyclopentyl)oxy) | 10.34 (s, 1H), 7.69 (d, J = 8.8 Hz, 2H), 7.33 (dd, | |
| | phenyl)-7-oxo-6,7- | J = 10.0, 8.8 Hz, 4H), 6.99 (d, J = 8.8 Hz, 2H), | |
| | dihydro-2H- | 6.42 (dd, J = 17.0, 10.0 Hz, 1H), 6.27 (dd, J = | |
| | pyrazolo[3,4- | 17.0, 2.0 Hz, 1H), 5.79 (dd, J = 10.0, 2.0 Hz, | |
| | d]pyridazin-2- | 1H), 5.39-5.19 (m, 1H), 5.06-4.98 (m, 1H), | |
| | yl)phenyl)acrylamide | 4.79 (s, 2H), 2.40-2.31 (m, 1H), 2.15-2.09 (m, | |
| | | 1H), 2.05-1.77 (m, 4H). | |
| 174 | N-(4-(4-amino-3-(4- | ¹H NMR (400 MHz, DMSO) δ 11.31 (s, 1H), | 493 |
| | ((3,3-difluoro- | 10.35 (s, 1H), 7.70 (d, J = 8.9 Hz, 2H), 7.35 (dd, | |
| | cyclopentyl)oxy)phenyl)- | J = 16.3, 8.8 Hz, 4H), 7.01 (d, J = 8.8 Hz, 2H), | |
| | 7-oxo-6,7- | 6.43 (dd, J = 17.0, 10.1 Hz, 1H), 6.28 (dd, J = | |
| | dihydro-2H- | 17.0, 2.0 Hz, 1H), 5.79 (dd, J = 10.1, 2.0 Hz, | |
| | pyrazolo[3,4- | 1H), 5.01 (s, 1H), 4.81 (s, 2H), 2.66 (tt, J = 15.8, | |
| | d]pyridazin-2- | 7.8 Hz, 1H), 2.33-2.10 (m, 4H), 2.02-1.88 (m, | |
| | yl)phenyl)acrylamide | 1H). | |
| 175 | N-(4-(4-amino-3-(4-((3- | ¹H NMR (400 MHz, DMSO) δ 11.30 (s, 1H), | 485 |
| | methylcyclohexyl)oxy) | 10.34 (s, 1H), 7.69 (d, J = 8.8 Hz, 2H), 7.32 (dd, | |
| | phenyl)-7-oxo-6,7- | J = 8.8, 2.6 Hz, 4H), 7.01 (d, J = 8.8 Hz, 2H), | |
| | dihydro-2H- | 6.42 (dd, J = 17.0, 10.0 Hz, 1H), 6.27 (dd, J = | |
| | pyrazolo[3,4- | 17.0, 2.0 Hz, 1H), 5.79 (dd, J = 10.0, 2.0 Hz, | |
| | d]pyridazin-2- | 1H), 4.80 (s, 2H), 4.74-4.27 (m, 1H), 2.07- | |
| | yl)phenyl)acrylamide | 2.00 (m, 1H), 1.89-1.69 (m, 2H), 1.67-1.47 | |
| | | (m, 3H), 1.35-1.16 (m, 2H), 1.05-0.94 (m, | |
| | | 1H), 0.89 (dd, J = 18.0, 6.8 Hz, 3H). | |
| 176 | N-(4-(4-amino-3-(4- | ¹H NMR (400 MHz, DMSO) δ 11.29 (s, 1H), | 389 |
| | hydroxyphenyl)-7-oxo- | 10.35 (s, 1H), 10.02 (s, 1H), 7.69 (d, J = 8.9 Hz, | |
| | 6,7-dihydro-2H- | 2H), 7.31 (d, J = 8.9 Hz, 2H), 7.23 (d, J = 8.5 | |
| | pyrazolo[3,4- | Hz, 2H), 6.83 (d, J = 8.6 Hz, 2H), 6.43 (dd, J = | |
| | d]pyridazin-2- | 17.0, 10.0 Hz, 1H), 6.28 (dd, J = 17.0, 1.9 Hz, | |
| | yl)phenyl)acrylamide | 1H), 5.79 (dd, J = 10.0, 1.9 Hz, 1H), 4.80 (s, 2H). | |
| 177 | N-(4-(4-amino-3-(4- | ¹H NMR (400 MHz, DMSO) δ 11.31 (s, 1H), | 433 |
| | (methoxymethoxy)phe- | 10.34 (s, 1H), 7.69 (d, J = 8.9 Hz, 2H), 7.35 (dd, | |
| | nyl)-7-oxo-6,7-dihydro- | J = 19.5, 8.8 Hz, 4H), 7.09 (d, J = 8.7 Hz, 2H), | |
| | 2H-pyrazolo[3,4- | 6.42 (dd, J = 17.0, 10.1 Hz, 1H), 6.27 (dd, J = | |
| | d]pyridazin-2- | 17.0, 1.9 Hz, 1H), 5.79 (dd, J = 10.0, 1.9 Hz, | |
| | yl)phenyl)acrylamide | 1H), 5.24 (s, 2H), 4.79 (s, 2H), 3.39 (s, 3H). | |
| 178 | N-(4-(4-amino-3-(1- | ¹H NMR (400 MHz, DMSO) δ 11.29 (s, 1H), | 516 |
| | (3,3-difluoro- | 10.31 (s, 1H), 7.71-7.61 (m, 5H), 7.35 (d, J = | |
| | cyclopentyl)-1H-indol- | 8.9 Hz, 2H), 7.17 (d, J = 8.6 Hz, 1H), 6.58 (d, | |
| | 5-yl)-7-oxo-6,7- | J = 3.1 Hz, 1H), 6.40 (dd, J = 16.9, 10.1 Hz, 1H), | |
| | dihydro-2H-pyrazolo | 6.25 (dd, J = 17.1, 1.8 Hz, 1H), 5.77 (dd, J = | |
| | [3,4-d]pyridazin-2- | 10.1, 1.7 Hz, 1H), 5.26-5.13 (m, 1H), 4.76 (s, | |
| | yl)phenyl)acrylamide | 2H), 2.79 (t, J = 9.7 Hz, 1H), 2.42 (d, J = 10.2 | |
| | | Hz, 3H), 2.20 (dd, J = 31.2, 15.8 Hz, 2H). | |
| 179 | 4-(4-amino-6-(6- | ¹H NMR (400 MHz, DMSO) δ 8.66 (t, J = 6.5 | 482 |
| | ethynyl-4-methyl- | Hz, 1H), 8.29 (s, 1H), 8.23 (s, 1H), 7.74 (d, J = | |
| | pyridin-3-yl)pyrazolo | 7.9 Hz, 1H), 7.51 (s, 1H), 7.38-7.25 (m, 2H), | |
| | [5,1-f][1,2,4]triazin-5- | 7.08 (s, 1H), 6.88 (d, J = 8.1 Hz, 1H), 4.37 (s, | |
| | yl)-2-methoxy-N- | 1H), 4.15-4.05 (m, 2H), 3.76 (s, 3H), 2.07 (s, | |
| | (2,2,2-trifluoroethyl) | 3H). | |
| | benzamide | | |
| 180 | 4-(4-amino-7-cyano-2- | ¹H NMR (400 MHz, DMSO) δ 8.67 (t, J = 6.4 | 505 |
| | (6-ethynylpyridin-3-yl)- | Hz, 1H), 8.47 (d, J = 2.0 Hz, 1H), 8.25 (s, 1H), | |
| | 1-methyl-1H- | 7.85 (m, 1H), 7.70 (d, J = 7.6 Hz, 1H), 7.63 (d, | |
| | pyrrolo[3,2-c]pyridin-3- | J = 8.0 Hz, 1H), 7.01 (s, 1H), 6.89 (m, 1H), 4.45 | |
| | yl)-2-methoxy-N- | (s, 1H), 4.14-4.03 (m, 2H), 3.85 (s, 3H), 3.76 | |
| | (2,2,2-trifluoroethyl) | (s, 3H). | |
| | benzamide | | |
| 181 | 4-(4-amino-7-cyano-2- | ¹H NMR (400 MHz, DMSO) δ 8.46 (d, J = 1.6 | 463 |
| | (6-ethynylpyridin-3-yl)- | Hz, 1H), 8.24 (s, 1H), 8.09 (d, J = 4.4 Hz, 1H), | |
| | 1-methyl-1H- | 7.86 (m, 1H), 7.61 (m, 2H), 6.95 (s, 1H), 6.86 | |
| | pyrrolo[3,2-c]pyridin-3- | (m, 1H), 4.46 (s, 1H), 3.84 (s, 3H), 3.71 (s, 3H), | |
| | yl)-N-cyclopropyl-2- | 2.81 (m, 1H), 0.71-0.64 (m, 2H), 0.55-0.49 | |
| | methoxybenzamide | (m, 2H). | |
| 182 | N-(4-(4-amino-5-(4- | ¹H NMR (400 MHz, DMSO) δ 10.21 (s, 1H), | 471 |
| | (cyclopentyloxy)-3- | 8.12 (s, 1H), 7.61 (d, J = 8.8 Hz, 2H), 7.43 (d, | |
| | methoxyphenyl)pyrazolo[5,1- | J = 8.8 Hz, 2H), 7.06-6.99 (m, 2H), 6.90-6.89 (m, | |
| | f][1,2,4]triazin-6- | 1H), 6.46-6.40 (m, 1H), 6.27-6.23 (m, 1H), 5.77- | |
| | yl)phenyl)acrylamide | 5.74 (m, 1H), 4.85 (s, 1H), 3.68 (s, 3H), 2.00- | |
| | | 1.88 (m, 2H), 1.78-1.75 (m, 4H), 1.60 (s, 2H). | |
| 183 | 4-(4-amino-6-(4-(2- | ¹H NMR (400 MHz, DMSO) δ 10.36 (s, 1H), | 448 |
| | fluoroacrylamido)phe- | 8.16 (s, 1H), 7.93-7.83 (m, 1H), 7.68 (m, 3H), | |
| | nyl)pyrazolo[5,1- | 7.61 (s, 1H), 7.41 (d, J = 8.8 Hz, 2H), 7.21 (s, | |
| | f][1,2,4]triazin-5-yl)-2- | 1H), 7.03-7.01 (m, 1H), 5.77-5.65 (m, 1H), | |
| | methoxybenzamide | 5.46-5.41 (m, 1H), 3.83 (s, 3H). | |
| 184 | 4-(6-(4- | ¹H NMR (400 MHz, DMSO) δ 10.23 (s, 1H), | 502 |
| | acrylamidophenyl)-4- | 8.46 (d, J = 6.9 Hz, 1H), 8.16 (s, 1H), 7.73 (d, | |
| | aminopyrazolo[5,1- | J = 7.8 Hz, 1H), 7.62 (d, J = 8.6 Hz, 2H), 7.41 (d, | |
| | f][1,2,4]triazin-5-yl)-N- | J = 8.6 Hz, 2H), 7.20 (s, 1H), 7.03 (d, J = 7.8 Hz, | |
| | (3-fluorocyclobutyl)-2- | 1H), 6.43 (dd, J = 17.0, 10.1 Hz, 1H), 6.25 (dd, | |
| | methoxybenzamide | J = 17.0, 1.7 Hz, 1H), 5.76 (dd, J = 10.1, 1.6 Hz, | |
| | | 1H), 5.28 (dd, J = 56.6, 3.6 Hz, 1H), 4.56 (d, J = | |
| | | 7.8 Hz, 1H), 3.81 (s, 3H), 2.54 (m, 2H), 2.48- | |
| | | 2.40 (m, 2H). | |
| 185 | 4-(4-amino-6-(4-(2- | ¹H NMR (400 MHz, DMSO) δ 10.37 (s, 1H), | 520 |
| | fluoroacrylamido)phe- | 8.46 (d, J = 6.9 Hz, 1H), 8.17 (s, 1H), 7.71 (dd, | |
| | nyl)pyrazolo[5,1- | J = 17.3, 8.2 Hz, 3H), 7.42 (d, J = 8.5 Hz, 2H), | |
| | f][1,2,4]triazin-5-yl)-N- | 7.20 (s, 1H), 7.03 (d, J = 7.8 Hz, 1H), 5.77 (d, | |
| | (3-fluorocyclobutyl)-2- | J = 3.7 Hz, 0.5H), 5.65 (d, J = 3.6 Hz, 0.5H), 5.44 | |
| | methoxybenzamide | (dd, J = 15.6, 3.5 Hz, 1H), 5.28 (d, J = 57.0 Hz, | |
| | | 1H), 4.56 (d, J = 8.2 Hz, 1H), 3.81 (s, 3H), 2.54 | |
| | | (m, 2H), 2.48-2.40 (m, 2H). | |
| 186 | 4-(4-amino-6-(4-(2- | ¹H NMR (400 MHz, DMSO) δ 10.36 (s, 1H), | 502 |
| | fluoroacrylamido)phe- | 8.32 (s, 1H), 8.16 (s, 1H), 7.78 (d, J = 7.8 Hz, | |
| | nyl)pyrazolo[5,1- | 1H), 7.68 (d, J = 8.8 Hz, 2H), 7.41 (d, J = 8.4 | |
| | f][1,2,4]triazin-5-yl)-2- | Hz, 2H), 7.18 (s, 1H), 7.01 (d, J = 8.0 Hz, 1H), | |
| | methoxy-N-(1- | 5.71 (m, 1H), 5.43 (m, 1H), 3.81 (s, 3H), 1.40 (s, | |
| | methylcyclopropyl)benzamide | 3H), 0.75 (m, 2H), 0.63 (m, 2H). | |
| 187 | 4-(6-(4-acrylamido-2- | ¹H NMR (400 MHz, DMSO) δ 10.44 (s, 1H), | 504 |
| | chlorophenyl)-4- | 8.26-8.17 (m, 1H), 8.09-8.00 (m, 1H), 7.99- | |
| | aminopyrazolo[5,1- | 7.89 (m, 1H), 7.77-7.60 (m, 1H), 7.59-7.49 | |
| | f][1,2,4]triazin-5-yl)-N- | (m, 1H), 7.43-7.36 (m, 1H), 7.04-6.92 (m, | |
| | cyclopropyl-2- | 1H), 6.92-6.83 (m, 1H), 6.48-6.24 (m, 2H), | |
| | methoxybenzamide | 5.90-5.76 (m, J = 8.6 Hz, 1H), 3.70 (s, 3H), | |
| | | 2.86-2.77 (m, 1H), 0.76-0.62 (m, 2H), 0.60- | |
| | | 0.44 (m, 2H). | |
| 188 | 4-(2-(4- | ¹H NMR (400 MHz, DMSO) δ 10.30 (s, 1H), | 557 |
| | acrylamidophenyl)-4- | 8.47 (s, 1H), 8.36 (s, 1H), 7.71 (d, J = 8.4 Hz, | |
| | amino-7-cyano-1- | 2H), 7.63 (d, J = 7.6 Hz, 1H), 7.30 (d, J = 8.4 | |
| | methyl-1H-pyrrolo[3,2- | Hz, 2H), 6.96 (s, 1H), 6.88 (d, J = 8.0 Hz, 1H), | |
| | c]pyridin-3-yl)-N-(3,3- | 6.46-6.40 (m, 1H), 6.29-6.25 (m, 1H), 5.78-5.77 | |
| | difluorocyclobutyl)-2- | (m, 1H), 4.24 (s, 1H), 3.84 (s, 3H), 3.72 (s, 3H), | |
| | methoxybenzamide | 2.95-2.88 (m, 2H), 2.73-2.71 (m, 2H). | |
| 189 | 4-(2-(4- | ¹H NMR (400 MHz, DMSO) δ 10.27 (s, 1H), | 513 |
| | acrylamidophenyl)-4- | 8.33 (t, J = 5.6 Hz, 1H), 8.22 (s, 1H), 7.75-7.64 | |
| | amino-7-cyano-1- | (m, 3H), 7.30 (d, J = 8.4 Hz, 2H), 6.96 (s, 1H), | |
| | methyl-1H-pyrrolo[3,2- | 6.89 (d, J = 7.6 Hz, 1H), 6.46-6.39 (m, 1H), | |
| | c]pyridin-3-yl)-N-(2- | 6.33-5.84 (m, 3H), 5.79-5.76 (m, 1H), 4.57 (t, | |
| | fluoroethyl)-2- | J = 5.2 Hz, 1H), 4.45 (t, J = 5.2 Hz, 1H), 3.81 (s, | |
| | methoxybenzamide | 3H), 3.74 (s, 3H), 3.62-3.58 (m, 1H), 3.56-3.52 | |
| | | (m, 1H). | |
| 190 | N-(4-(4-amino-7- | ¹H NMR (400 MHz, DMSO) δ 10.32 (s, 1H), | 520 |
| | cyano-3-(3-fluoro-4- | 8.47 (d, J = 5.0 Hz, 1H), 8.35 (s, 1H), 7.74 (d, | |
| | ((4-methylpyrimidin-2- | J = 8.5 Hz, 2H), 7.37 (t, J = 8.3 Hz, 1H), 7.33 (d, | |
| | yl)oxy)phenyl)-1- | J = 8.4 Hz, 2H), 7.26 (d, J = 11.1 Hz, 1H), 7.19 (d, | |
| | methyl-1H-pyrrolo[3,2- | J = 5.0 Hz, 1H), 7.12 (d, J = 8.3 Hz, 1H), 6.44 | |
| | c]pyridin-2- | (dd, J = 16.9, 10.0 Hz, 1H), 6.28 (d, J = 15.4 Hz, | |
| | yl)phenyl)acrylamide | 1H), 5.79 (d, J = 11.6 Hz, 1H), 3.84 (s, 3H), 2.41 | |
| | | (s, 3H). | |
| 191 | N-(4-(4-amino-7- | ¹H NMR (400 MHz, DMSO) δ 10.30 (s, 1H), | 531 |
| | cyano-3-(3-methoxy-4- | 8.36 (s, 1H), 7.73 (d, J = 8.6 Hz, 2H), 7.69-7.63 | |
| | ((6-methylpyridin-2- | (m, 1H), 7.32 (d, J = 8.6 Hz, 2H), 7.09 (d, J = 8.0 | |
| | yl)oxy)phenyl)-1- | Hz, 1H), 6.99 (d, J = 1.8 Hz, 1H), 6.93 (d, J = | |
| | methyl-1H-pyrrolo[3,2- | 7.3 Hz, 1H), 6.83 (dd, J = 8.0, 1.9 Hz, 1H), 6.64 | |
| | c]pyridin-2- | (d, J = 8.2 Hz, 1H), 6.44 (dd, J = 17.0, 10.1 Hz, | |
| | yl)phenyl)acrylamide | 1H), 6.27 (dd, J = 17.0, 1.9 Hz, 1H), 5.78 (dd, | |
| | | J = 10.1, 1.9 Hz, 1H), 3.87 (s, 3H), 3.56 (s, 3H), | |
| | | 2.28 (s, 3H) | |
| 192 | 4-(2-(1-(1- | ¹H NMR (400 MHz, DMSO) δ 8.68 (t, J = 6.4 | 607 |
| | acryloylpiperidin-4-yl)- | Hz, 1H), 8.18 (s, 1H), 7.98 (s, 1H), 7.76 (d, J = | |
| | 1H-pyrazol-4-yl)-4- | 7.9 Hz, 1H), 7.43 (s, 1H), 7.03 (s, 1H), 6.98 (d, | |
| | amino-7-cyano-1- | J = 8.0 Hz, 1H), 6.84 (dd, J = 16.8, 10.5 Hz, 1H), | |
| | methyl-1H-pyrrolo[3,2- | 6.14-6.09 (m, 1H), 5.69 (dd, J = 10.4, 2.3 Hz, | |
| | c]pyridin-3-yl)-2- | 1H), 4.47 (m, 2H), 4.11 (m, 3H), 3.87 (s, 3H), | |
| | methoxy-N-(2,2,2- | 3.80 (s, 3H), 3.23 (m, 1H), 2.82 (m, 1H), 2.03 | |
| | trifluoroethyl)benzamide | (m, 2H), 1.76 (m, 2H). | |
| 193 | 4-(4-amino-6-(4-(2- | ¹H NMR (400 MHz, DMSO) δ 10.36 (s, 1H), | 524 |
| | fluoroacrylamido)phe- | 8.47 (s, 2H), 8.17 (s, 1H), 7.79 (d, J = 7.8 Hz, | |
| | nyl)pyrazolo[5,1- | 1H), 7.69 (d, J = 8.4 Hz, 2H), 7.42 (d, J = 8.4 | |
| | f][1,2,4]triazin-5-yl)-N- | Hz, 2H), 7.23 (s, 1H), 7.05 (d, J = 7.8 Hz, 1H), | |
| | (2,2- | 5.78-5.65 (m, 2H), 5.46-5.42 (m, 1H), 3.82 (s, | |
| | difluorocyclopropyl)-2- | 3H), 3.50 (s, 1H), 2.04-1.90 (m, 1H), 1.80- | |
| | methoxybenzamide | 1.66 (m, 1H). | |
| 194 | 4-(6-(4- | ¹H NMR (400 MHz, DMSO) δ 10.23 (s, 1H), | 476 |
| | acrylamidophenyl)-4- | 8.44 (t, J = 5.6 Hz, 2H), 8.16 (s, 1H), 7.87 (d, J = | |
| | aminopyrazolo[5,1- | 7.8 Hz, 1H), 7.63 (d, J = 8.5 Hz, 2H), 7.40 (d, | |
| | f][1,2,4]triazin-5-yl)-N- | J = 8.5 Hz, 2H), 7.23 (s, 1H), 7.04 (d, J = 7.9 Hz, | |
| | (2-fluoroethyl)-2- | 1H), 6.46-6.40 (m, 1H), 6.25 (d, J = 17.0 Hz, | |
| | methoxybenzamide | 1H), 5.77-5.68 (m, 2H), 4.62 (t, J = 5.0 Hz, | |
| | | 1H), 4.50 (t, J = 5.0 Hz, 1H), 3.84 (s, 3H), 3.69- | |
| | | 3.65 (m, 1H), 3.62-3.58 (m, 1H). | |
| 195 | 4-(4-amino-6-(4-(2- | ¹H NMR (400 MHz, DMSO) δ 10.36 (s, 1H), | 494 |
| | fluoroacrylamido)phe- | 8.45-8.34 (m, 2H), 8.17 (s, 1H), 7.87 (d, J = 7.8 | |
| | nyl)pyrazolo[5,1- | Hz, 1H), 7.68 (d, J = 8.6 Hz, 2H), 7.41 (d, J = | |
| | f][1,2,4]triazin-5-yl)-N- | 8.6 Hz, 2H), 7.23 (s, 1H), 7.03 (d, J = 7.9 Hz, | |
| | (2-fluoroethyl)-2- | 1H), 5.77-5.65 (m, 2H), 5.46-5.41 (m, 1H), | |
| | methoxybenzamide | 4.62 (t, J = 5.2 Hz, 1H), 4.50 (t, J = 5.2 Hz, 1H), | |
| | | 3.84 (s, 3H), 3.68-3.64 (m, 1H), 3.62-3.58 (m, 1H). | |
| 196 | 4-(6-(4- | ¹H NMR (400 MHz, DMSO) δ 10.22 (s, 1H), | 494 |
| | acrylamidophenyl)-4- | 8.55 (t, J = 6.0 Hz, 1H), 8.16 (s, 1H), 7.88 (d, J = | |
| | aminopyrazolo[5,1- | 7.6 Hz, 1H), 7.62 (d, J = 8.4 Hz, 2H), 7.39 (d, | |
| | f][1,2,4]triazin-5-yl)-N- | J = 8.4 Hz, 2H), 7.24 (s, 1H), 7.04 (d, J = 8.0 Hz, | |
| | (2,2-difluoroethyl)-2- | 1H), 6.43 (m, 1H), 6.33-5.96 (m, 2H), 5.76 (m, | |
| | methoxybenzamide | 1H), 3.85 (s, 3H), 3.75 (m, 2H). | |
| 197 | 4-(4-amino-6-(4-(2- | ¹H NMR (400 MHz, DMSO) δ 10.36 (s, 1H), | 512 |
| | fluoroacrylamido)phe- | 8.55 (t, J = 6.0 Hz, 1H), 8.17 (s, 1H), 7.88 (d, J = | |
| | nyl)pyrazolo[5,1- | 7.6 Hz, 1H), 7.68 (d, J = 8.4 Hz, 2H), 7.41 (d, | |
| | f][1,2,4]triazin-5-yl)-N- | J = 8.4 Hz, 2H), 7.24 (s, 1H), 7.04 (d, J = 8.0 Hz, | |
| | (2,2-difluoroethyl)-2- | 1H), 6.16 (m, 1H), 5.76 (s, 2H), 5.43 (m, 1H), | |
| | methoxybenzamide | 3.85 (s, 3H), 3.75 (s, 2H). | |
| 198 | 4-(2-(4- | ¹H NMR (400 MHz, DMSO) δ 10.27 (s, 1H), | 531 |
| | acrylamidophenyl)-4- | 8.45 (t, J = 6.0 Hz, 1H), 8.21 (s, 1H), 7.71 (m, | |
| | amino-7-cyano-1- | 3H), 7.29 (d, J = 8.4 Hz, 2H), 6.96 (s, 1H), 6.89 | |
| | methyl-1H-pyrrolo[3,2- | (d, J = 7.6 Hz, 1H), 6.43 (m, 1H), 6.32-5.94 (m, | |
| | c]pyridin-3-yl)-N-(2,2- | 4H), 5.77 (d, J = 10.0 Hz, 1H), 3.81 (s, 3H), 3.74 | |
| | difluoroethyl)-2- | (s, 3H), 3.68 (m, 2H). | |
| | methoxybenzamide | | |
| 199 | 4-(4-amino-6-(2-chloro- | ¹H NMR (400 MHz, DMSO) δ 10.54 (s, 1H), | 522 |
| | 4-(2- | 8.21 (s, 1H), 8.05 (d, J = 4.3 Hz, 1H), 7.94 (d, | |
| | fluoroacrylamido)phe- | J = 2.0 Hz, 1H), 7.78-7.67 (m, 1H), 7.67-7.57 | |
| | nyl)pyrazolo[5,1- | (m, 1H), 7.48-7.36 (m, 1H), 7.02-6.92 (m, | |
| | f][1,2,4]triazin-5-yl)-N- | 1H), 6.90-6.81 (m, 1H), 5.75 (dd, J = 47.6, 3.7 | |
| | cyclopropyl-2- | Hz, 1H), 5.48 (dd, J = 15.6, 3.7 Hz, 1H), 3.69 (s, | |
| | methoxybenzamide | 3H), 2.91-2.72 (m, 1H), 0.76-0.61 (m, 2H), | |
| | | 0.59-0.46 (m, 2H). | |
| 200 | 4-(2-(1- | ¹H NMR (400 MHz, MeOD) δ 8.07-7.95 (m, | 527 |
| | acryloylpyrrolidin-3- | 2H), 7.22-7.10 (m, 2H), 6.58-6.18 (m, 1H), | |
| | yl)-4-amino-7-cyano-1- | 6.17-6.08 (m, 1H), 5.74-5.45 (m, 1H), 4.16 | |
| | methyl-1H-pyrrolo[3,2- | (m, 2H), 4.09 (s, 3H), 3.98 (m, 3H), 3.94-3.78 | |
| | c]pyridin-3-yl)-2- | (m, 2H), 3.69 (m, 1H), 3.54 (m, 2H), 2.35-2.23 | |
| | methoxy-N-(2,2,2- | (m, 1H), 2.13-2.01 (m, 1H). | |
| | trifluoroethyl)benzamide | | |
| 201 | 4-(6-(1-(1- | ¹H NMR (400 MHz, DMSO) δ 8.75 (t, J = 6.4 | 570 |
| | acryloylpiperidin-4-yl)- | Hz, 1H), 8.12 (s, 1H), 7.93-7.85 (m, 2H), 7.34 | |
| | 1H-pyrazol-4-yl)-4- | (s, 1H), 7.25 (s, 1H), 7.13 (d, J = 7.8 Hz, 1H), | |
| | aminopyrazolo[5,1- | 6.83 (dd, J = 16.7, 10.5 Hz, 1H), 6.10 (dd, J = | |
| | f][1,2,4]triazin-5-yl)-2- | 16.7, 2.2 Hz, 1H), 5.68 (dd, J = 10.5, 2.1 Hz, | |
| | methoxy-N-(2,2,2- | 1H), 4.47 (d, J = 11.5 Hz, 2H), 4.23-4.07 (m, | |
| | trifluoroethyl)benzamide | 3H), 3.88 (s, 3H), 3.19 (t, J = 12.3 Hz, 1H), 2.79 | |
| | | (t, J = 12.5 Hz, 1H), 2.00 (d, J = 11.7 Hz, 2H), | |
| | | 1.78 (dd, J = 21.7, 11.0 Hz, 2H). | |
| 202 | N-(4-(4-amino-7-oxo-3- | ¹H NMR (400 MHz, DMSO) δ 11.34 (s, 1H), | 456 |
| | (4-(pyrrolidin-1- | 10.36 (s, 1H), 8.21 (s, 1H), 7.68 (d, J = 8.8 Hz, | |
| | ylmethyl)phenyl)-6,7- | 2H), 7.40 (s, 4H), 7.31 (d, J = 8.7 Hz, 2H), 6.42 | |
| | dihydro-2H- | (dd, J = 17.0, 10.1 Hz, 1H), 6.27 (dd, J = 16.9, | |
| | pyrazolo[3,4- | 1.4 Hz, 1H), 5.78 (dd, J = 10.2, 1.3 Hz, 1H), 4.78 | |
| | d]pyridazin-2- | (s, 2H), 3.64 (s, 2H), 2.45 (s, 4H), 1.70 (s, 4H). | |
| | yl)phenyl)acrylamide | | |
| 203 | 4-amino-3-(4- | ¹H NMR (400 MHz, DMSO) δ 11.38 (s, 1H), | 427 |
| | (cyclohexyloxy)phenyl)- | 8.52 (d, J = 1.9 Hz, 1H), 7.87 (dd, J = 8.3, 2.3 | |
| | 2-(6-ethynylpyridin-3- | Hz, 1H), 7.69 (d, J = 8.4 Hz, 1H), 7.38 (d, J = | |
| | yl)-2,6-dihydro-7H- | 8.5 Hz, 2H), 7.05 (d, J = 8.6 Hz, 2H), 4.87 (s, | |
| | pyrazolo[3,4- | 2H), 4.51 (s, 1H), 4.41 (s, 1H), 1.93 (t, J = 11.5 | |
| | d]pyridazin-7-one | Hz, 2H), 1.79-1.67 (m, 2H), 1.59-1.50 (m, | |
| | | 1H), 1.41 (dq, J = 22.5, 11.3 Hz, 4H), 1.25 (d, | |
| | | J = 10.4 Hz, 1H). | |
| 204 | 4-(4-amino-6-(4-(2- | ¹H NMR (400 MHz, DMSO) δ 10.36 (s, 1H), | 520 |
| | fluoroacrylamido)phe- | 8.50 (t, J = 5.9 Hz, 1H), 8.17 (s, 1H), 7.84 (d, J = | |
| | nyl)pyrazolo[5,1- | 7.8 Hz, 1H), 7.75-7.66 (m, 2H), 7.46-7.38 (m, | |
| | f][1,2,4]triazin-5-yl)-N- | 2H), 7.24 (d, J = 1.3 Hz, 1H), 7.04 (dd, J = 7.8, | |
| | ((1- | 1.4 Hz, 1H), 5.65-5.77 (m, 1H), 5.43 (dd, J = | |
| | fluorocyclopropyl)methyl)- | 15.6, 3.7 Hz, 1H), 3.85 (s, 3H), 3.76 (dd, J = | |
| | 2-methoxybenzamide | 20.7, 5.9 Hz, 2H), 1.11-0.95 (m, 2H), 0.90- | |
| | | 0.75 (m, 2H). | |
| 205 | N-(4-(4-amino-5-(3- | ¹H NMR (400 MHz, DMSO) δ 10.24 (s, 1H), | 483 |
| | fluoro-4-((4- | 8.53 (d, J = 5.0 Hz, 1H), 7.66 (d, J = 8.7 Hz, | |
| | methylpyrimidin-2- | 2H), 7.53-7.43 (m, 2H), 7.42 (s, 1H), 7.40 (s, | |
| | yl)oxy)phenyl)pyrazolo | 0H), 7.30-7.27 (m, 1H), 7.23 (d, J = 5.0 Hz, | |
| | [5,1-f][1,2,4]triazin-6- | 1H), 6.43 (dd, J = 17.0, 10.1 Hz, 1H), 6.26 (dd, | |
| | yl)phenyl)acrylamide | J = 17.0, 2.0 Hz, 1H), 5.77 (dd, J = 10.1, 2.0 Hz, | |
| | | 1H), 2.45 (s, 3H). | |
| 206 | N-(4-(4-amino-5-(4-cyclobutoxy- | ¹H NMR (400 MHz, DMSO) δ 8.13 (s, 1H), 7.68- | 445 |
| | phenyl)pyrazolo[5,1- | 7.63 (m, 2H), 7.42-7.36 (m, 2H), 7.34-7.29 | |
| | f][1,2,4]triazin-6- | (m, 2H), 7.01-6.92 (m, 2H), 5.76 (d, J = 3.6 Hz, | |
| | yl)phenyl)-2- | 0.5H), 5.64 (d, J = 3.6 Hz, 0.5H), 5.42 (dd, J = | |
| | fluoroacrylamide | 15.6, 3.6 Hz, 1H), 4.79-4.72 (m, 1H), 2.48- | |
| | | 2.42 (m, 2H), 2.15-2.04 (m, 2H), 1.85-1.61 | |
| | | (m, 2H). | |
| 207 | N-(4-(4-amino-5-(4-cyclobutoxy- | ¹H NMR (400 MHz, DMSO) δ 9.84 (s, 1H), 8.12 | 441 |
| | phenyl)pyrazolo[5,1- | (s, 1H), 7.61 (d, J = 8.8 Hz, 2H), 7.39-7.34 (m, | |
| | f][1,2,4]triazin-6- | 2H), 7.34-7.28 (m, 2H), 6.97 (d, J = 8.7 Hz, | |
| | yl)phenyl)methacrylamide | 2H), 5.78 (s, 1H), 5.52 (s, 1H), 4.80-4.70 (m, | |
| | | 1H), 2.45 (m, 2H), 2.15-2.04 (m, 2H), 1.93 (s, | |
| | | 3H), 1.73 (m, 2H). | |
| 208 | N-(4-(4-amino-5-(4- | ¹H NMR (400 MHz, MeOD) δ 8.06 (s, 1H), 7.59 | 445 |
| | cyclobutoxy-3- | (d, J = 8.7 Hz, 2H), 7.44 (d, J = 8.8 Hz, 2H), | |
| | fluorophenyl)pyrazolo[5,1- | 7.23-7.05 (m, 3H), 6.42 (dd, J = 16.9, 9.5 Hz, | |
| | f][1,2,4]triazin-6- | 1H), 6.35 (dd, J = 16.9, 2.3 Hz, 1H), 5.77 (dd, | |
| | yl)phenyl)acrylamide | J = 9.5, 2.3 Hz, 1H), 2.57-2.49 (m, 2H), 2.30- | |
| | | 2.16 (m, 2H), 1.90 (q, J = 10.4 Hz, 1H), 1.76 | |
| | | (dd, J = 18.8, 8.1 Hz, 1H). | |
| 209 | 4-(6-(1- | ¹H NMR (400 MHz, DMSO) δ 8.72 (s, 1H), 8.12 | 490 |
| | acryloylpyrrolidin-3- | (d, J = 2.8 Hz, 1H), 7.93-7.88 (m, 1H), 7.23 (s, | |
| | yl)-4- | 1H), 7.14 (d, J = 7.6 Hz, 1H), 6.63-6.51 (m, | |
| | aminopyrazolo[5,1- | 1H), 6.12 (dd, J = 16.8 Hz, J = 2.4 Hz, 1H), 5.65 | |
| | f][1,2,4]triazin-5-yl)-2- | (t, J = 10 Hz, 1H), 4.20-4.11 (m, 2H), 3.94 (s, | |
| | methoxy-N-(2,2,2- | 3H), 3.81-3.72 (m, 2H), 3.64-3.54 (m, 2H), | |
| | trifluoroethyl)benzamide | 3.49-3.37 (m, 1H), 2.24-2.07 (m, 2H) | |
| 210 | 4-(2-(4- | ¹H NMR (400 MHz, DMSO) δ 10.28 (s, 1H), | 539 |
| | acrylamidophenyl)-4- | 8.40 (t, J = 5.9 Hz, 1H), 8.21 (s, 1H), 7.78-7.61 | |
| | amino-7-cyano-1- | (m, 3H), 7.35-7.26 (m, 2H), 6.97 (d, J = 1.3 Hz, | |
| | methyl-1H-pyrrolo[3,2- | 1H), 6.89 (dd, J = 7.9, 1.4 Hz, 1H), 6.47-6.38 | |
| | c]pyridin-3-yl)-N-((1- | (m, 1H), 6.30-6.23 (m, 1H), 5.78 (dd, J = 10.1, | |
| | fluorocyclopropyl)methyl)- | 2.0 Hz, 1H), 3.81 (s, 3H), 3.74 (s, 3H), 3.74- | |
| | 2-methoxybenzamide | 3.66 (m, 2H), 1.06-0.90 (m, 2H), 0.85-0.73 | |
| | | (m, 2H). | |
| 211 | 4-(2-(4-acrylamido-3- | ¹H NMR (400 MHz, DMSO) δ 10.07 (s, 1H), | 525 |
| | fluorophenyl)-4-amino- | 8.22 (s, 1H), 8.09-8.05 (m, 2H), 7.60 (d, J = 7.6 | |
| | 7-cyano-1-methyl-1H- | Hz, 1H), 7.35-7.32 (m, 1H), 7.17-7.15 (m, | |
| | pyrrolo[3,2-c]pyridin-3- | 1H), 6.96 (d, J = 1.2 Hz, 1H), 6.90-6.87 (m, | |
| | yl)-N-cyclopropyl-2- | 1H), 6.66-6.59 (m, 1H), 6.30-6.07 (m, 3H), | |
| | methoxybenzamide | 5.80-5.77 (m, 1H), 3.82 (s, 3H), 3.72 (s, 3H), | |
| | | 2.83-2.78 (m, 1H), 0.68-0.66 (m, 2H), 0.53- | |
| | | 0.51 (m, 2H). | |
| 212 | 4-(2-(4-acrylamido-3- | ¹H NMR (400 MHz, DMSO) δ 10.06 (s, 1H), | 567 |
| | fluorophenyl)-4-amino- | 8.66 (t, J = 6.4 Hz, 1H), 8.23 (s, 1H), 8.08 (t, J = | |
| | 7-cyano-1-methyl-1H- | 8.2 Hz, 1H), 7.71 (d, J = 8.0 Hz, 1H), 7.36-7.32 | |
| | pyrrolo[3,2-c]pyridin-3- | (m, 1H), 7.18-7.15 (m, 1H), 7.02 (d, J = 1.6 Hz, | |
| | yl)-2-methoxy-N- | 1H), 6.93-6.90 (m, 1H), 6.66-6.59 (m, 1H), | |
| | (2,2,2- | 6.44-5.86 (m, 3H), 5.81-5.78 (m, 1H), 4.11- | |
| | trifluoroethyl)benzamide | 4.07 (m, 2H), 3.83 (s, 3H), 3.77 (s, 3H). | |
| 213 | 4-(2-(1-acryloyl-2,5- | ¹H NMR (400 MHz, MeOD) δ 8.11 (d, J = 1.3 | 525 |
| | dihydro-1H-pyrrol-3- | Hz, 1H), 7.99 (dd, J = 7.9, 4.6 Hz, 1H), 7.19 (d, | |
| | yl)-4-amino-7-cyano-1- | J = 1.4 Hz, 1H), 7.16-7.11 (m, 1H), 6.61-6.34 | |
| | methyl-1H-pyrrolo[3,2- | (m, 1H), 6.30-6.17 (m, 2H), 5.74 (m, 1H), 4.59 | |
| | c]pyridin-3-yl)-2- | (dd, J = 5.8, 3.6 Hz, 1H), 4.41 (d, J = 2.1 Hz, | |
| | methoxy-N-(2,2,2- | 1H), 4.34 (s, 1H), 4.16 (m, 3H), 4.05 (d, J = 1.9 | |
| | trifluoroethyl)benzamide | Hz, 3H), 3.97 (d, J = 2.9 Hz, 3H). | |
| 214 | N-(4-(4-amino-3- | ¹H NMR (400 MHz, DMSO) δ 10.54 (s, 1H), | 414 |
| | bromo-7-cyano-1- | 8.17 (s, 1H), 7.93 (d, J = 8.4 Hz, 2H), 7.51 (d, | |
| | methyl-1H-pyrrolo[3,2- | J = 8.8 Hz, 2H), 7.00 (s, 2H), 5.84-5.71 (m, 1H), | |
| | c]pyridin-2-yl)phenyl)- | 5.51-5.47 (m, 1H), 3.78 (s, 3H). | |
| | 2-fluoroacrylamide | | |
| 215 | 1-(4- | ¹H NMR (400 MHz, DMSO) δ 10.21 (s, 1H), | 485 |
| | acrylamidophenyl)-3- | 7.67 (t, 1H), 7.59 (d, J = 8.9 Hz, 2H), 7.18-7.01 | |
| | amino-5-(3-methoxy-4- | (m, 5H), 6.94 (d, J = 6.6 Hz, 2H), 6.67 (d, J = 8.2 | |
| | ((6-methylpyridin-2- | Hz, 1H), 6.40 (dd, J = 16.9, 10.1 Hz, 1H), 6.24 | |
| | yl)oxy)phenyl)-1H- | (dd, J = 17.0, 2.0 Hz, 1H), 5.75 (dd, J = 10.1, 2.0 | |
| | pyrazole-4- | Hz, 1H), 5.67 (s, 2H), 5.45 (s, 1H), 3.58 (s, 3H), | |
| | carboxamide | 2.27 (s, 3H). | |
| 216 | N-(4-(4-amino-7-oxo-3- | ¹H NMR (400 MHz, DMSO) δ 11.24 (s, 1H), | 442 |
| | (4-(pyrrolidin-1- | 10.35 (s, 1H), 7.69 (d, J = 8.9 Hz, 2H), 7.33 (d, | |
| | yl)phenyl)-6,7-dihydro- | J = 8.9 Hz, 2H), 7.17 (d, J = 8.7 Hz, 2H), 6.57 (d, | |
| | 2H-pyrazolo[3,4- | J = 8.8 Hz, 2H), 6.43 (dd, J = 16.9, 10.1 Hz, 1H), | |
| | d]pyridazin-2- | 6.27 (dd, J = 17.0, 2.0 Hz, 1H), 5.79 (dd, J = | |
| | yl)phenyl)acrylamide | 10.1, 1.9 Hz, 1H), 4.83 (s, 2H), 3.24 (d, J = 6.2 | |
| | | Hz, 4H), 1.95 (t, J = 6.4 Hz, 4H). | |
| 217 | N-(4-(4-amino-3-(3- | ¹H NMR (400 MHz, DMSO) δ 10.35 (s, 1H), | 494 |
| | methoxy-4-((6- | 8.30 (s, 1H), 7.73 (d, J = 8.9 Hz, 2H), 7.71-7.65 | |
| | methylpyridin-2- | (m, 1H), 7.37 (d, J = 8.9 Hz, 2H), 7.22 (d, J = 1.8 | |
| | yl)oxy)phenyl)-2H- | Hz, 1H), 7.19 (d, J = 8.1 Hz, 1H), 7.00 (dd, J = | |
| | pyrazolo[3,4- | 8.0, 1.9 Hz, 1H), 6.96 (d, J = 7.3 Hz, 1H), 6.71 | |
| | d]pyrimidin-2- | (d, J = 8.1 Hz, 1H), 6.44 (dd, J = 17.0, 10.1 Hz, | |
| | yl)phenyl)acrylamide | 1H), 6.28 (dd, J = 17.0, 1.9 Hz, 1H), 5.79 (dd, | |
| | | J = 10.1, 1.9 Hz, 1H), 3.59 (s, 3H), 2.31 (s, 3H). | |
| 218 | N-(4-(4-amino-5-(6- | ¹H NMR (400 MHz, DMSO) δ 10.23 (s, 1H), | 428 |
| | cyclobutoxypyridin-3- | 8.14-8.10 (m, 2H), 7.71-7.60 (m, 3H), 7.39- | |
| | yl)pyrazolo[5,1- | 7.31 (m, 2H), 6.93-6.82 (m, 1H), 6.46-6.40 | |
| | f][1,2,4]triazin-6- | (m, 1H), 6.28-6.23 (m, 1H), 5.78-5.75 (m, | |
| | yl)phenyl)acrylamide | 1H), 5.29-5.13 (m, 1H), 2.45-2.38 (m, 2H), | |
| | | 2.17-2.06 (m, 2H), 1.84-1.76 (m, 1H), 1.68- | |
| | | 1.61 (m, 1H). | |
| 219 | N-(4-(4-amino-5-(4-cyclo- | ¹H NMR (400 MHz, DMSO) δ 10.43 (s, 1H), | 445 |
| | butoxyphenyl)pyrazolo[5,1- | 8.15 (s, 1H), 7.64 (d, J = 13.0 Hz, 1H), 7.39- | |
| | f][1,2,4]triazin-6-yl)-3- | 7.33 (m, 2H), 7.19 (m, 2H), 6.89 (d, J = 8.7 Hz, | |
| | fluorophenyl)acrylamide | 2H), 6.42 (m, 1H), 6.28 (dd, J = 17.0, 2.0 Hz, | |
| | | 1H), 5.81 (dd, J = 10.0, 2.0 Hz, 1H), 4.73-4.64 | |
| | | (m, 1H), 2.46-2.40 (m, 2H), 2.11-2.01 (m, | |
| | | 2H), 1.82-1.58 (m, 2H). | |
| 220 | N-(4-(4-amino-5-(4-cyclo- | ¹H NMR (400 MHz, DMSO) δ 10.24 (s, 1H), | 457 |
| | butoxyphenyl)pyrazolo[5,1- | 8.11 (s, 1H), 7.40 (m, 2H), 7.25 (m, 2H), 7.13 (d, | |
| | f][1,2,4]triazin-6-yl)-3- | J = 8.7 Hz, 2H), 6.86 (d, J = 8.7 Hz, 2H), 6.43 | |
| | methoxyphenyl)acrylamide | (dd, J = 17.0, 10.1 Hz, 1H), 6.26 (dd, J = 17.0, | |
| | | 2.0 Hz, 1H), 5.77 (dd, J = 10.1, 2.0 Hz, 1H), 4.71- | |
| | | 4.64 (m, 1H), 2.45-2.39 (m, 2H), 2.08-1.99 | |
| | | (m, 2H), 1.80-1.58 (m, 2H). | |
| 221 | N-(4-(4-amino-5-(4- | ¹H NMR (400 MHz, Methanol-d4) δ 8.05 (s, | 427 |
| | (cyclopropylmethoxy)phe- | 1H), 7.61-7.52 (m, 2H), 7.50-7.44 (m, 2H), | |
| | nyl)pyrazolo[5,1- | 7.39-7.31 (m, 2H), 7.16-7.02 (m, 2H), 6.42 | |
| | f][1,2,4]triazin-6- | (dd, J = 16.9, 9.6 Hz, 1H), 6.34 (dd, J = 17.0, 2.3 | |
| | yl)phenyl)acrylamide | Hz, 1H), 5.76 (dd, J = 9.5, 2.4 Hz, 1H), 3.91 (d, | |
| | | J = 6.9 Hz, 2H), 1.42-1.22 (m, 1H), 0.73-0.57 | |
| | | (m, 2H), 0.39 (dt, J = 6.2, 4.5 Hz, 2H). | |
| 222 | N-(4-(4-amino-5-(4-cyclo- | ¹H NMR (400 MHz, DMSO) δ 10.22 (s, 1H), | 413 |
| | propoxyphenyl)pyrazolo[5,1- | 8.13 (s, 1H), 7.61 (d, J-7.7 Hz, 2H), 7.38 (m, | |
| | f][1,2,4]triazin-6- | 4H), 7.19 (d, J = 7.4 Hz, 2H), 6.43 (m, 1H), 6.25 | |
| | yl)phenyl)acrylamide | (d, J = 17.0 Hz, 1H), 5.76 (d, J = 10.0 Hz, 1H), | |
| | | 3.92 (m, 1H), 0.82 (d, J = 5.1 Hz, 2H), 0.73 (m, 2H). | |
| 223 | N-(4-(4-amino-5-(4- | ¹H NMR (400 MHz, DMSO) δ 10.21 (s, 1H), | 471 |
| | (cyclohexylthio)phenyl) | 8.14 (s, 1H), 7.60 (d, J = 8.8 Hz, 2H), 7.46 (d, | |
| | pyrazolo[5,1- | J = 8.4 Hz, 2H), 7.35 (d, J = 8.4 Hz, 4H), 6.46- | |
| | f][1,2,4]triazin-6- | 6.39 (m, 1H), 6.27-6.23 (m, 1H), 5.82-5.70 | |
| | yl)phenyl)acrylamide | (m, 1H), 3.42-3.35 (m, 1H), 2.04-1.92 (m, | |
| | | 2H), 1.76-1.72 (m, 2H), 1.61-1.58 (m, 1H), | |
| | | 1.45-1.23 (m, 5H). | |
| 224 | N-(4-(4-amino-3- | ¹H NMR (400 MHz, DMSO) δ 10.39 (s, 1H), | 396 |
| | bromo-7-cyano-1- | 8.17 (s, 1H), 7.86 (d, J = 8.7 Hz, 2H), 7.48 (d, | |
| | methyl-1H-pyrrolo[3,2- | J = 8.7 Hz, 2H), 6.99 (s, 2H), 6.48 (dd, J = 17.0, | |
| | c]pyridin-2- | 10.1 Hz, 1H), 6.31 (dd, J = 17.0, 2.0 Hz, 1H), | |
| | yl)phenyl)acrylamide | 5.81 (dd, J = 10.1, 2.0 Hz, 1H), 3.78 (s, 3H). | |
| 225 | N-(4-(4-amino-5-(4- | ¹H NMR (400 MHz, DMSO) δ 10.22 (s, 1H), | 455 |
| | (2,2,2- | 8.14 (s, 1H), 7.60 (d, J = 8.8 Hz, 2H), 7.41-7.37 | |
| | trifluoroethoxy)phenyl) | (m, 4H), 7.21-7.18 (m, 2H), 6.42 (dd, J = 16.8, | |
| | pyrazolo[5,1- | 10.0 Hz, 1H), 6.25 (dd, J = 17.2, 2.0 Hz, 1H), | |
| | f][1,2,4]triazin-6- | 5.76 (dd, J = 10, 2.0 Hz, 1H), 4.85 (q, J = 8.8 Hz, | |
| | yl)phenyl)acrylamide | 2H). | |
| 226 | N-(4-(4-amino-5-(4-iso- | ¹H NMR (400 MHz, DMSO) δ 10.21 (s, 1H), | 415 |
| | propoxyphenyl)pyrazolo[5,1- | 8.31 (s, 1H), 8.12 (s, 1H), 7.60 (d, J = 8.7 Hz, | |
| | f][1,2,4]triazin-6- | 2H), 7.40 (d, J = 8.7 Hz, 2H), 7.31 (d, J = 8.6 | |
| | yl)phenyl)acrylamide | Hz, 2H), 7.05 (d, J = 8.7 Hz, 2H), 6.46-6.39 (m, | |
| | | 1H), 6.27-6.23 (m, 1H), 5.77-5.74 (m, 1H), | |
| | | 5.35 (s, 1H), 4.78-4.62 (m, 1H), 1.32 (d, J = 6.0 | |
| | | Hz, 6H). | |
| 227 | N-(4-(4-amino-5-(4- | ¹H NMR (400 MHz, DMSO) δ 10.22 (s, 1H), | 429 |
| | (oxetan-3- | 8.31 (s, 1H), 8.13 (s, 1H), 7.60 (d, J = 8.8 Hz, | |
| | yloxy)phenyl)pyrazolo[5,1- | 2H), 7.37-7.33 (m, 4H), 6.98-6.77 (m, 2H), | |
| | f][1,2,4]triazin-6- | 6.46-6.39 (m, 1H), 6.27-6.23 (m, 1H), 5.78- | |
| | yl)phenyl)acrylamide | 5.75 (m, 1H), 5.38-5.34 (m, 2H), 4.97-4.94 | |
| | | (m, 2H), 4.63-4.60 (m, 2H). | |
| 228 | N-(4-(4-amino-5-(4- | ¹H NMR (400 MHz, DMSO) δ 10.22 (s, 1H), | 455 |
| | (cyclohexyloxy)phenyl) | 8.12 (s, 1H), 7.60 (d, J = 8.8 Hz, 2H), 7.47-7.37 | |
| | pyrazolo[5,1- | (m, 2H), 7.31 (d, J = 8.7 Hz, 2H), 7.07 (d, J = 8.7 | |
| | f][1,2,4]triazin-6- | Hz, 2H), 6.42 (dd, J = 17.0, 10.1 Hz, 1H), 6.25 | |
| | yl)phenyl)acrylamide | (dd, J = 17.0, 2.0 Hz, 1H), 5.76 (dd, J = 10.1, 2.0 | |
| | | Hz, 1H), 4.45-4.38 (m, 1H), 2.00 (d, J = 9.6 Hz, | |
| | | 2H), 1.79-1.70 (m, 2H), 1.61-1.22 (m, 6H). | |
| 229 | N-(4-(4-amino-5-(4- | ¹H NMR (400 MHz, DMSO) δ 10.21 (s, 1H), | 457 |
| | ((tetrahydro-2H-pyran- | 8.32 (s, 1H), 8.13 (s, 1H), 7.60 (d, J = 8.7 Hz, | |
| | 4- | 2H), 7.39 (d, J = 8.7 Hz, 2H), 7.33 (d, J = 8.5 | |
| | yl)oxy)phenyl)pyrazolo | Hz, 2H), 7.12 (d, J = 8.6 Hz, 2H), 6.46-6.39 (m, | |
| | [5,1-f][1,2,4]triazin-6- | 1H), 6.27-6.23 (m, 1H), 5.77-5.74 (m, 1H), | |
| | yl)phenyl)acrylamide | 5.38 (s, 1H), 4.73-4.61 (m, 1H), 3.90-3.86 (m, | |
| | | 2H), 3.58-3.46 (m, 2H), 2.03 (d, J = 9.8 Hz, | |
| | | 2H), 1.67-1.61 (m, 2H). | |
| 230 | N-(4-(4-amino-5-(4-((3- | ¹H NMR (400 MHz, DMSO) δ 10.21 (s, 1H), | 469 |
| | methylcyclohexyl)oxy) | 8.12 (s, 1H), 7.60 (d, J = 8.8 Hz, 2H), 7.40 (dd, | |
| | phenyl)pyrazolo[5,1- | J = 8.8, 2.0 Hz, 2H), 7.31 (dd, J = 8.8, 2.4 Hz, | |
| | f][1,2,4]triazin-6- | 2H), 7.06 (dd, J = 8.8, 1.6 Hz, 2H), 6.42 (dd, J = | |
| | yl)phenyl)acrylamide | 16.0, 10.0 Hz, 1H), 6.25 (dd, J = 16.8, 2.0 Hz, | |
| | | 1H), 5.76 (dd, J = 10.4, 2.0 Hz, 1H), 4.46 (dd, | |
| | | J = 84.0, 74.4 Hz, 1H), 2.11 (d, J = 11.2 Hz, 1H), | |
| | | 1.95-1.71 (m, 2H), 1.58 (dd, J = 41.6, 13.2 Hz, | |
| | | 2H), 1.39 (d, J = 12.8 Hz, 1H), 1.33-1.20 (m, | |
| | | 1H), 1.08-0.97 (m, 1H), 0.92 (dd, J = 16.4, 6.4 | |
| | | Hz, 4H). | |
| 231 | N-(4-(4-amino-5-(4- | ¹H NMR (400 MHz, DMSO) δ 10.21 (s, 1H), | 483 |
| | ((4,4-dimethyl- | 8.12 (s, 1H), 7.60 (d, J = 8.8 Hz, 2H), 7.39 (d, | |
| | cyclohexyl)oxy)phe- | J = 8.8 Hz, 2H), 7.31 (d, J = 8.8 Hz, 2H), 7.06 (d, | |
| | nyl)pyrazolo[5,1- | J = 8.8 Hz, 2H), 6.42 (dd, J = 16.8, 10.0 Hz, 1H), | |
| | f][1,2,4]triazin-6- | 6.25 (dd, J = 16.8, 2.0 Hz, 1H), 5.76 (dd, J = | |
| | yl)phenyl)acrylamide | 10.0, 2.0 Hz, 1H), 4.53-4.18 (m, 1H), 1.87 (dd, | |
| | | J = 13.2, 4.4 Hz, 2H), 1.62 (td, J = 13.2, 3.6 Hz, | |
| | | 2H), 1.47 (dd, J = 13.6, 4.4 Hz, 2H), 1.36-1.27 | |
| | | (m, 2H), 0.95 (d, J = 8.0 Hz, 6H). | |
| 232 | N-(4-(6-(4- | ¹H NMR (400 MHz, DMSO) δ 10.23 (s, 1H), | 470 |
| | acrylamidophenyl)-4- | 9.55 (s, 1H), 8.70-7.89 (m, 3H), 7.62 (d, J = 8.7 | |
| | aminopyrazolo[5,1- | Hz, 2H), 7.43 (d, J = 8.8 Hz, 2H), 7.10 (d, J = | |
| | f][1,2,4]triazin-5-yl)-2- | 1.7 Hz, 1H), 6.95-6.92 (m, 1H), 6.46-6.40 (m, | |
| | methoxyphenyl)cyclo- | 1H), 6.28-6.23 (m, 1H), 5.77-5.74 (m, 1H), | |
| | propanecarboxamide | 5.49 (s, 1H), 3.79 (s, 3H), 2.15 (d, J = 4.8 Hz, | |
| | | 1H), 0.84-0.73 (m, 4H). | |
| 233 | N-(4-(4-amino-5-(4-(1- | ¹H NMR (400 MHz, DMSO) δ 10.21 (s, 1H), | 524 |
| | (cyclopropylamino)- | 8.16 (s, 1H), 7.67 (d, J = 7.6 Hz, 1H), 7.59 (d, | |
| | 2,2,2-trifluoroethyl)-3- | J = 8.8 Hz, 2H), 7.38 (d, J = 8.8 Hz, 2H), 7.15 (s, | |
| | methoxyphenyl)pyrazolo[5,1- | 1H), 7.06 (d, J = 7.6 Hz, 1H), 6.41 (dd, J = 17.2, | |
| | f][1,2,4]triazin-6- | 10.4 Hz, 1H), 6.25 (dd, J = 16.8, 2.0 Hz, 1H), | |
| | yl)phenyl)acrylamide | 5.76 (dd, J = 10.2, 2.0 Hz, 1H), 4.86-4.73 (m, | |
| | | 1H), 3.77 (s, 3H), 3.64 (d, J = 10.8 Hz, 1H), 2.07 | |
| | | (td, J = 6.0, 2.4 Hz, 1H), 0.46-0.25 (m, 4H). | |
| 234 | 4-(4-amino-6-(2-fluoro- | ¹H NMR (400 MHz, MeOD) δ 8.14 (d, J = 0.8 | 556 |
| | 4-(2-fluoroacryl- | Hz, 1H), 7.83 (d, J = 7.6 Hz, 1H), 7.62 (d, J = | |
| | amido)phenyl)pyrazolo[ | 12.4 Hz, 1H), 7.50-7.42 (m, 2H), 7.05 (d, J = | |
| | 5,1-f][1,2,4]triazin-5- | 10.8 Hz, 2H), 5.73 (dd, J = 46.4, 3.5 Hz, 1H), | |
| | yl)-N-(3,3- | 5.32 (dd, J = 15.2, 3.6 Hz, 1H), 4.33 (d, J = 7.2 | |
| | difluorocyclobutyl)-2- | Hz, 1H), 3.81 (s, 3H), 3.03-2.94 (m, 2H), 2.75- | |
| | methoxybenzamide | 2.63 (m, 2H). | |
| 235 | 4-(4-amino-7-cyano-2- | ¹H NMR (400 MHz, DMSO) δ 12.48 (s, 1H), | 511 |
| | (4-(2- | 10.36 (s, 1H), 8.18-8.11 (m, 2H), 7.70-7.64 | |
| | fluoroacrylamido)phe- | (m, 3H), 7.38-7.33 (m, 2H), 7.08 (d, J = 1.2 Hz, | |
| | nyl)-1H-pyrrolo[3,2- | 1H), 6.96 (dd, J = 7.8, 1.4 Hz, 1H), 6.59-5.48 | |
| | c]pyridin-3-yl)-N- | (m, 3H), 5.44 (dd, J = 15.6, 3.7 Hz, 1H), 3.77 (s, | |
| | cyclopropyl-2- | 3H), 2.88-2.80 (m, 1H), 0.73-0.67 (m, 2H), | |
| | methoxybenzamide | 0.58-0.54 (m, 2H). | |
| 236 | 4-(2-(4- | ¹H NMR (400 MHz, DMSO) δ 10.28 (s, 1H), | 539 |
| | acrylamidophenyl)-4- | 8.22 (s, 1H), 8.05 (d, J = 4.4 Hz, 1H), 7.69 (d, | |
| | amino-7-cyano-1-(2- | J = 8.4 Hz, 2H), 7.56 (d, J = 7.6 Hz, 1H), 7.33 (d, | |
| | fluoroethyl)-1H- | J = 8.4 Hz, 2H), 6.95 (d, J = 1.2 Hz, 1H), 6.88 | |
| | pyrrolo[3,2-c]pyridin-3- | (m, 1H), 6.43 (m, 1H), 6.27 (m, 1H), 5.78 (m, | |
| | yl)-N-cyclopropyl-2- | 1H), 4.61 (m, 2H), 4.52 (s, 2H), 3.70 (s, 3H), | |
| | methoxybenzamide | 2.79 (m, 1H), 0.66 (m, 2H), 0.54-0.48 (m, 2H). | |
| 237 | N-(4-(2-(4- | ¹H NMR (400 MHz, DMSO) δ 10.27 (s, 1H), | 507 |
| | acrylamidophenyl)-4- | 9.41 (s, 1H), 8.18 (s, 1H), 7.92 (d, J = 8.1 Hz, | |
| | amino-7-cyano-1- | 1H), 7.68 (d, J = 8.6 Hz, 2H), 7.30 (d, J = 8.5 | |
| | methyl-1H-pyrrolo[3,2- | Hz, 2H), 6.86 (d, J = 1.6 Hz, 1H), 6.79-6.76 (m, | |
| | c]pyridin-3-yl)-2- | 1H), 6.46-6.40 (m, 1H), 6.29-6.24 (m, 1H), | |
| | methoxyphenyl)cyclo- | 5.79-5.76 (m, 1H), 3.80 (s, 3H), 3.70 (s, 3H), | |
| | propanecarboxamide | 2.06 (d, J = 4.9 Hz, 1H), 0.76-0.74 (m, 4H). | |
| 238 | N-(4-(4-amino-7- | ¹H NMR (400 MHz, DMSO) δ 10.29 (s, 1H), | 506 |
| | cyano-3-(4-((5- | 8.75 (s, 2H), 8.20 (s, 1H), 7.70 (d, J = 8.7 Hz, | |
| | fluoropyrimidin-2- | 2H), 7.33-7.30 (m, 4H), 7.24-7.21 (m, 2H), | |
| | yl)oxy)phenyl)-1- | 6.41 (d, J = 10.1 Hz, 1H), 6.27 (dd, J = 17.0, 2.0 | |
| | methyl-1H-pyrrolo[3,2- | Hz, 1H), 5.78 (dd, J = 10.1, 2.0 Hz, 1H), 3.80 (s, | |
| | c]pyridin-2- | 3H). | |
| | yl)phenyl)acrylamide | | |
| 239 | N-(4-(4-amino-5-(4- | ¹H NMR (400 MHz, DMSO) δ 10.22 (s, 1H), | 477 |
| | ((3,3-difluoro- | 8.32 (s, 1H), 8.13 (s, 1H), 7.60 (d, J = 8.8 Hz, | |
| | cyclopentyl)oxy)phe- | 2H), 7.39 (d, J = 8.7 Hz, 2H), 7.34 (d, J = 8.7 | |
| | nyl)pyrazolo[5,1- | Hz, 2H), 7.06 (d, J = 8.7 Hz, 2H), 6.46-6.39 (m, | |
| | f][1,2,4]triazin-6- | 1H), 6.28-6.23 (m, 1H), 5.78-5.75 (m, 1H), | |
| | yl)phenyl)acrylamide | 5.38 (s, 1H), 5.04 (s, 1H), 2.72-2.67 (m, 1H), | |
| | | 2.36-2.16 (m, 4H), 2.07-1.98 (m, 1H). | |
| 240 | N-(4-(4-amino-5-(4- | ¹H NMR (400 MHz, DMSO) δ 10.22 (s, 1H), | 457 |
| | cyclobutoxy-3- | 8.12 (s, 1H), 7.61 (d, J = 8.8 Hz, 2H), 7.43 (d, | |
| | methoxyphenyl)pyrazolo[5,1- | J = 8.8 Hz, 2H), 7.01 (d, J = 1.8 Hz, 1H), 6.90- | |
| | f][1,2,4]triazin-6- | 6.86 (m, 2H), 6.43 (dd, J = 17.2, 10.0 Hz, 1H), | |
| | yl)phenyl)acrylamide | 6.25 (dd, J = 17.2, 2.0 Hz, 1H), 5.76 (dd, J = | |
| | | 10.0, 2.0 Hz, 1H), 4.76-4.65 (m, 1H), 3.69 (s, | |
| | | 3H), 2.47-2.40 (m, 2H), 2.15-2.07 (m, 2H), | |
| | | 1.84-1.76 (m, 1H), 1.71-1.58 (m, 1H). | |
| 241 | N-(4-(4-amino-5-(4-(1- | ¹H NMR (400 MHz, DMSO) δ 10.34 (s, 1H), | 542 |
| | (cyclopropylamino)- | 8.17 (s, 1H), 7.66 (d, J = 8.8 Hz, 3H), 7.39 (d, | |
| | 2,2,2-trifluoroethyl)-3- | J = 8.7 Hz, 2H), 7.15 (s, 1H), 7.06 (d, J = 7.8 Hz, | |
| | methoxyphenyl)pyrazolo[5,1- | 1H), 5.70 (dd, J = 47.6, 3.6 Hz, 1H), 5.43 (dd, | |
| | f][1,2,4]triazin-6- | J = 15.6, 3.6 Hz, 1H), 4.79 (dd, J = 17.9, 8.8 Hz, | |
| | yl)phenyl)-2- | 1H), 3.77 (s, 3H), 3.64 (d, J = 10.7 Hz, 1H), 2.07 | |
| | fluoroacrylamide | (s, 1H), 0.48-0.25 (m, 4H). | |
| 242 | 4-(2-(4- | ¹H NMR (400 MHz, DMSO) δ 10.29 (s, 1H), | 521 |
| | acrylamidophenyl)-4- | 8.23 (s, 1H), 8.05 (d, J = 4.3 Hz, 1H), 7.71 (d, | |
| | amino-7-cyano-1-ethyl- | J = 8.6 Hz, 2H), 7.56 (d, J = 7.8 Hz, 1H), 7.35 (d, | |
| | 1H-pyrrolo[3,2- | J = 8.5 Hz, 2H), 6.95 (d, J = 1.2 Hz, 1H), 6.89- | |
| | c]pyridin-3-yl)-N- | 6.87 (m, 1H), 6.47-6.40 (m, 1H), 6.30-6.25 | |
| | cyclopropyl-2- | (m, 1H), 5.80-5.77 (m, 1H), 4.24-4.19 (m, | |
| | methoxybenzamide | 2H), 3.69 (s, 3H), 2.82-2.77 (m, 1H), 1.23 (t, | |
| | | J = 7.0 Hz, 3H), 0.69-0.63 (m, 2H), 0.53-0.49 | |
| | | (m, 2H). | |
| 243 | N-(4-(4-amino-7-cyano- | ¹H NMR (400 MHz, DMSO) δ 10.29 (s, 1H), | 520 |
| | 3-(4-((5-fluoro-4- | 8.55 (d, J = 1.3 Hz, 1H), 8.19 (s, 1H), 7.70 (d, | |
| | methylpyrimidin-2-yl) | J = 8.6 Hz, 2H), 7.31 (d, J = 8.4 Hz, 4H), 7.22- | |
| | oxy)phenyl)-1-methyl- | 7.17 (m, 2H), 6.48-6.39 (m, 1H), 6.27 (dd, J = | |
| | 1H-pyrrolo[3,2-c] | 17.0, 2.0 Hz, 1H), 5.78 (dd, J = 10.1, 2.0 Hz, | |
| | pyridine-2-yl)phenyl) | 1H), 3.80 (s, 3H), 2.40 (d, J = 2.5 Hz, 3H). | |
| | acrylamide | | |
| 244 | N-(4-(4-amino-7- | ¹H NMR (400 MHz, DMSO) δ 10.30 (s, 1H), | 561 |
| | cyano-3-(4-(1- | 8.37 (s, 1H), 7.70 (d, J = 8.6 Hz, 2H), 7.53 (d, | |
| | (cyclopropylamino)- | J = 7.8 Hz, 1H), 7.29 (t, J = 8.2 Hz, 2H), 6.92-6.87 | |
| | 2,2,2-trifluoroethyl)-3- | (m, 2H), 6.46-6.39 (m, 1H), 6.28-6.24 (m, 1H), | |
| | methoxyphenyl)-1- | 5.78 (dd, J = 10.4, 2.0 Hz, 1H), 4.73 (d, J = 7.2 | |
| | methyl-1H-pyrrolo[3,2- | Hz, 1H), 3.85 (s, 3H), 3.69 (s, 3H), 2.00 (s, 1H), | |
| | c]pyridin-2- | 0.39-0.27 (m, 4H). | |
| | yl)phenyl)acrylamide | | |
| 245 | N-(4-(4-amino-5-(4- | ¹H NMR (400 MHz, DMSO) δ 10.22 (s, 1H), | 491 |
| | ((4,4-difluoro- | 8.13 (s, 1H), 7.60 (d, J = 8.8 Hz, 2H), 7.39 (d, | |
| | cyclohexyl)oxy)phenyl) | J = 8.8 Hz, 4H), 7.34 (d, J = 8.4 Hz, 2H), 7.13 (d, | |
| | pyrazolo[5,1-f][1,2,4] | J = 8.4 Hz, 2H), 6.42 (m, 1H), 6.25 (m, 1H), | |
| | triazin-6-yl)phenyl) | 5.76 (m, 1H), 4.69 (s, 1H), 2.11-1.94 (m, 6H), | |
| | acrylamide | 1.84 (m, 2H). | |
| 246 | N-(4-(4-amino-5-(4-((1- | ¹H NMR (400 MHz, DMSO) δ 10.21 (s, 1H), | 442 |
| | methylazetidin-3- | 8.12 (s, 1H), 7.60 (d, J = 8.8 Hz, 2H), 7.35 (dd, | |
| | yl)oxy)phenyl)pyrazolo | J = 20.6, 8.8 Hz,4H), 6.96 (d, J = 8.7 Hz, 2H), | |
| | [5,1-f][1,2,4]triazin-6- | 6.40 (d, J = 10.1 Hz, 1H), 6.27 (d, J = 2.0 Hz, | |
| | yl)phenyl)acrylamide | 1H), 5.76 (d, J = 12.1 Hz, 1H), 4.82 (s, 1H), 3.78- | |
| | | 3.74 (m, 2H), 3.04-3.00 (m, 2H), 2.30 (s, 3H). | |
| 247 | N-(4-(4-amino-5-(4-(3- | ¹H NMR (400 MHz, MeOD) δ 8.05 (s, 1H), 7.57 | 471 |
| | (dimethylamino)cyclo- | (d, J = 8.4 Hz, 2H), 7.46 (d, J = 8.8 Hz, 2H), | |
| | butoxy)phenyl)pyrazolo[5,1- | 7.35 (d, J = 8.8 Hz, 2H), 6.98 (d, J = 8.4 Hz, | |
| | f][1,2,4]triazin-6- | 2H), 6.46-6.31 (m, 2H), 5.76 (d, J = 9.8 Hz, | |
| | yl)phenyl)acrylamide | 1H), 4.60 (s, 1H), 3.08 (s, 1H), 2.45-2.44 (m, | |
| | | 2H), 2.39-2.37 (m, 2H), 2.24 (s, 6H). | |
| 248 | 4-(2-(4-acrylamido- | ¹H NMR (400 MHz, DMSO) δ 12.48 (br s, 1H), | 535 |
| | phenyl)-4-amino-7- | 10.24 (s, 1H), 8.73 (t, J = 6.3 Hz, 1H), 8.14 (s, | |
| | cyano-1H-pyrrolo[3,2- | 1H), 7.79 (d, J = 7.8 Hz, 1H), 7.61 (d, J = 8.7 | |
| | c]pyridin-3-yl)-2- | Hz, 2H), 7.33 (d, J = 8.7 Hz, 2H), 7.13 (d, J = | |
| | methoxy-N-(2,2,2- | 1.0 Hz, 1H), 7.00 (dd, J = 7.9, 1.2 Hz, 1H), 6.52- | |
| | trifluoroethyl)benzamide | 6.37 (m, 1H), 6.36-5.39 (m, 4H), 4.17-4.08 | |
| | | (m, 2H), 3.81 (s, 3H). | |
| 249 | 4-(4-amino-6-(2-fluoro- | ¹H NMR (400 MHz, DMSO) δ 10.57 (s, 1H), | 538 |
| | 4-(2- | 8.39 (d, J = 6.8 Hz, 1H), 8.21 (s, 1H), 7.68-7.58 | |
| | fluoroacrylamido)phe- | (m, 3H), 7.45 (m, 1H), 7.06 (s, 1H), 6.91 (dd, J = | |
| | nyl)pyrazolo[5,1-f][1,2,4] | 7.8, 1.3 Hz, 1H), 5.75 (dd, J = 47.6, 3.7 Hz, 1H), | |
| | triazin-5-yl)-N-(3- | 5.48 (dd, J = 15.6, 3.7 Hz, 1H), 5.37-5.13 (m, | |
| | fluorocyclobutyl)-2- | 1H), 4.53 (d, J = 8.9 Hz, 1H), 3.75 (s, 3H), 2.49- | |
| | methoxybenzamide | 2.37 (m, 4H). | |
| 250 | 4-(4-amino-6-(4-(2- | ¹H NMR (400 MHz, DMSO) δ 10.37 (s, 1H), | 550 |
| | fluoroacrylamido)-2- | 8.40 (t, J = 5.8 Hz, 2H), 8.16 (s, 1H), 7.76 (d, J = | |
| | methoxyphenyl)pyrazolo[5,1- | 7.9 Hz, 1H), 7.44-7.42 (m, 2H), 7.35-7.27 (m, | |
| | f][1,2,4]triazin-5- | 1H), 7.00 (d, J = 1.1 Hz, 1H), 6.89-6.86 (m, | |
| | yl)-N-((1- | 1H), 6.04 (s, 1H), 5.79-5.66 (m, 1H), 5.48- | |
| | fluorocyclopropyl)methyl)-2- | 5.43 (m, 1H), 3.78-3.67 (m, 5H), 3.35 (s, 3H), | |
| | methoxybenzamide | 1.01-0.96 (m, 2H), 0.85-0.74 (m, 2H). | |
| 251 | 4-(4-amino-6-(4-(2- | ¹H NMR (400 MHz, MeOD) δ 8.11 (s, 1H), 7.95 | 542 |
| | fluoroacrylamido)-2- | (d, J = 8.0 Hz, 1H), 7.42 (d, J = 1.6 Hz, 1H), | |
| | methoxyphenyl)pyrazolo[5,1- | 7.40 (d, J = 8.4 Hz, 1H), 7.32 (dd, J = 8.4, 1.6 | |
| | f][1,2,4]triazin-5- | Hz, 1H), 7.04 (d, J = 8.0 Hz, 1H), 7.01 (s, 1H), | |
| | yl)-N-(2,2- | 6.19-5.84 (m, 1H), 5.72 (dd, J = 46.4, 3.6 Hz, | |
| | difluoroethyl)-2- | 1H), 5.31 (dd, J = 15.2, 3.6 Hz, 1H), 3.83-3.72 | |
| | methoxybenzamide | (m, 5H), 3.39 (s, 3H). | |
| 252 | 4-(4-amino-6-(6-(2- | ¹H NMR (400 MHz, DMSO) δ 10.74 (s, 1H), | 531 |
| | fluoroacrylamido)pyridin- | 8.74 (t, J = 6.4 Hz, 1H), 8.33 (d, J = 2.0 Hz, 1H), | |
| | 3-yl)pyrazolo[5,1-f] | 8.20 (s, 1H), 8.04 (d, J = 8.8 Hz, 1H), 7.90 (dd, | |
| | [1,2,4]triazin-5-yl)-2- | J = 8.8, 2.2 Hz, 1H), 7.85 (d, J = 8.0 Hz, 1H), 7.28 | |
| | methoxy-N-(2,2,2- | (s, 1H), 7.07 (d, J = 8.0 Hz, 1H), 5.84 (dd, J = | |
| | trifluoroethyl)benzamide | 47.2, 4.0 Hz, 1H), 5.49 (dd, J = 15.6, 4.0 Hz, | |
| | | 1H), 4.21-4.09 (m, 2H), 3.85 (s, 3H). | |
| 253 | 4-(4-amino-6-(3-fluoro- | ¹H NMR (400 MHz, DMSO) δ 10.20 (s, 1H), | 548 |
| | 4-(2-fluoroacrylamido) | 8.74 (s, 1H), 8.20 (s, 1H), 7.86 (d, J = 7.9 Hz, | |
| | phenyl)pyrazolo[5,1-f] | 1H), 7.49 (m, 1H), 7.32-7.23 (m, 3H), 7.08 (d, | |
| | [1,2,4]triazin-5-yl)-2- | J = 7.9 Hz, 1H), 5.77 (d, J = 3.8 Hz, 0.5H), 5.65 | |
| | methoxy-N-(2,2,2- | (d, J = 3.7 Hz, 0.5H), 5.45 (dd, J = 15.6, 3.8 Hz, | |
| | trifluoroethyl)benzamide | 1H), 4.15 (m, 2H), 3.86 (s, 3H). | |
| 254 | 4-(4-amino-6-(4-(2- | ¹H NMR (400 MHz, DMSO) δ 9.29 (s, 1H), 8.73 | 560 |
| | fluoroacrylamido)-3- | (s, 1H), 8.18 (s, 1H), 7.86 (d, J = 7.8 Hz, 1H), | |
| | methoxyphenyl)pyrazolo[5,1- | 7.72 (d, J = 8.3 Hz, 1H), 7.28 (d, J = 1.2 Hz, 1H), | |
| | f][1,2,4]triazin-5- | 7.14 (d, J = 1.7 Hz, 1H), 7.08 (dd, J = 7.9, 1.4 | |
| | yl)-2-methoxy-N- | Hz, 1H), 7.03 (dd, J = 8.2, 1.7 Hz, 1H), 5.76 (d, | |
| | (2,2,2-trifluoroethyl) | J = 3.8 Hz, 0.5H), 5.64 (d, J = 3.7 Hz, 0.5H), 5.43 | |
| | benzamide | (dd, J = 15.8, 3.7 Hz, 1H), 4.14 (m, 2H), 3.86 (s, | |
| | | 3H), 3.66 (s, 3H). | |
| 255 | 4-(4-amino-6-(4-(2- | ¹H NMR (400 MHz, DMSO) δ 10.37 (s, 1H), | 550 |
| | fluoroacrylamido)-2- | 8.34 (d, J = 7.0 Hz, 1H), 8.17 (s, 1H), 7.65 (d, | |
| | methoxyphenyl)pyrazolo[5,1- | J = 7.8 Hz, 1H), 7.43 (dd, J = 10.9, 2.7 Hz, 2H), | |
| | f][1,2,4]triazin-5- | 7.30 (dd, J = 8.1, 3.1 Hz, 1H), 6.97 (s, 1H), 6.87 | |
| | yl)-N-(3- | (dd, J = 7.8, 1.3 Hz, 1H), 5.72 (dd, J = 47.6, 3.6 | |
| | fluorocyclobutyl)-2- | Hz, 1H), 5.45 (dd, J = 15.6, 3.7 Hz, 1H), 5.36- | |
| | methoxybenzamide | 5.13 (m, 1H), 4.53 (m, 1H), 3.72 (s, 3H), 3.36 (s, | |
| | | 3H), 2.41 (m, 4H). | |
| 256 | 4-(4-amino-6-(2-fluoro- | ¹H NMR (400 MHz, DMSO) δ 10.57 (s, 1H), | 520 |
| | 4-(2-fluoroacrylamido) | 8.26 (s, 1H), 8.20 (s, 1H), 7.71 (d, J = 8.0 Hz, | |
| | phenyl)pyrazolo[5,1- | 1H), 7.65 (dd, J = 12.4, 2.0 Hz, 1H), 7.59 (dd, | |
| | f][1,2,4]triazin-5-yl)-2- | J = 8.4, 2.0 Hz, 1H), 7.43 (t, J = 8.4 Hz, 1H), 7.04 | |
| | methoxy-N-(1- | (d, J = 1.2 Hz, 1H), 6.88 (dd, J = 8.0, 1.2 Hz, | |
| | methylcyclopropyl)benzamide | 1H), 5.74 (dd, J = 47.6, 3.6 Hz, 1H), 5.48 (dd, | |
| | | J = 15.6, 3.6 Hz, 1H), 3.75 (s, 3H), 1.37 (s, 3H), | |
| | | 0.72 (t, J = 5.6 Hz, 2H), 0.66-0.54 (m, 2H). | |
| 257 | 4-(4-amino-6-(4-(2- | ¹H NMR (400 MHz, DMSO) δ 10.36 (s, 1H), | 532 |
| | fluoroacrylamido)-2- | 8.22 (s, 1H), 8.16 (s, 1H), 7.69 (d, J = 8.0 Hz, | |
| | methoxyphenyl)pyrazolo[5,1- | 1H), 7.46-7.39 (m, 2H), 7.29 (d, J = 8.0 Hz, | |
| | f][1,2,4]triazin-5- | 1H), 6.95 (d, J = 1.2 Hz, 1H), 6.85 (dd, J = 8.0, | |
| | yl)-2-methoxy-N-(1- | 1.4 Hz, 1H), 5.72 (dd, J = 47.6, 3.6 Hz, 1H), 5.45 | |
| | methylcyclopropyl)benzamide | (dd, J = 15.6, 3.6 Hz, 1H), 3.71 (s, 3H), 3.36 (s, | |
| | | 3H), 1.36 (s, 3H), 0.71 (t, J = 5.6 Hz, 2H), 0.60 | |
| | | (dd, J = 6.8, 4.8 Hz, 2H). | |
| 258 | 4-(4-amino-6-(2-fluoro- | ¹H NMR (400 MHz, DMSO) δ 10.57 (s, 1H), | 538 |
| | 4-(2-fluoroacrylamido) | 8.44 (t, J = 5.8 Hz, 2H), 8.21 (s, 1H), 7.77 (d, J = | |
| | phenyl)pyrazolo[5,1- | 7.9 Hz, 1H), 7.67-7.59 (m, 2H), 7.44 (t, J = 8.4 | |
| | f][1,2,4]triazin-5-yl)-N- | Hz, 1H), 7.10 (d, J = 1.2 Hz, 1H), 6.93-6.90 (m, | |
| | ((1-fluorocyclopropyl) | 1H), 6.07 (s, 1H), 5.81-5.68 (m, 1H), 5.51- | |
| | methyl)-2- | 5.46 (m, 1H), 3.84-3.67 (m, 5H), 0.99 (d, J = | |
| | methoxybenzamide | 19.2 Hz, 2H), 0.87-0.76 (m, 2H). | |
| 259 | 4-(4-amino-6-(4-(2- | ¹H NMR (400 MHz, DMSO) δ 10.37 (s, 1H), | 554 |
| | fluoroacrylamido)-2- | 8.35 (s, 2H), 8.17 (s, 1H), 7.71 (d, J = 7.9 Hz, | |
| | methoxyphenyl)pyrazolo[5,1- | 1H), 7.47-7.37 (m, 2H), 7.30 (d, J = 8.1 Hz, | |
| | f][1,2,4]triazin-5- | 1H), 6.99 (d, J = 1.1 Hz, 1H), 6.90-6.87 (m, | |
| | yl)-N-(2,2- | 1H), 6.01 (s, 1H), 5.79-5.66 (m, 1H), 5.48- | |
| | difluorocyclopropyl)-2- | 5.43 (m, 1H), 3.73 (s, 3H), 3.48-3.44 (m, 1H), | |
| | methoxybenzamide | 3.35 (s, 3H), 1.99-1.88 (m, 1H), 1.76-1.64 (m, 1H). | |
| 260 | N-(4-(4-amino-5-(4- | ¹H NMR (400 MHz, DMSO) δ 10.22 (s, 1H), | 453 |
| | (bicyclo[3.1.0]hexan-3- | 8.30 (br s, 1H), 8.12 (s, 1H), 7.60 (d, J = 8.6 Hz, | |
| | yloxy)phenyl)pyrazolo[5,1- | 2H), 7.39 (d, J = 8.6 Hz, 2H), 7.30 (d, J = 8.5 | |
| | f][1,2,4]triazin-6- | Hz, 2H), 7.04 (d, J = 8.5 Hz, 2H), 6.43 (dd, J = | |
| | yl)phenyl)acrylamide | 17.0, 10.1 Hz, 1H), 6.25 (d, J = 16.7 Hz, 1H), | |
| | | 5.76 (d, J = 11.7 Hz, 1H), 5.32 (br s, 1H), 4.65- | |
| | | 4.56 (m, 1H), 2.36 (dd, J = 12.6, 7.1 Hz, 2H), | |
| | | 1.88-1.80 (m, 2H), 1.37-1.32 (m, 2H), 0.44- | |
| | | 0.37 (m, 1H), 0.24-0.19 (m, 1H). | |
| 261 | N-(4-(4-amino-5-(4-((3- | ¹H NMR (400 MHz, DMSO) δ 10.21 (s, 1H), | 459 |
| | fluorocyclopentyl)oxy) | 8.13 (s, 1H), 7.60 (d, J = 8.7 Hz, 2H), 7.36 (dd, | |
| | phenyl)pyrazolo[5,1- | J = 26.8, 8.7 Hz, 4H), 7.04 (d, J = 8.7 Hz, 2H), | |
| | f][1,2,4]triazin-6- | 6.42 (dd, J = 17.0, 10.1 Hz, 1H), 6.25 (dd, J = | |
| | yl)phenyl)acrylamide | 17.0, 2.0 Hz, 1H), 5.78-5.74 (m, 1H), 5.33 (d, | |
| | | J = 51.6 Hz, 1H), 5.12-4.98 (m, 1H), 2.45-2.32 | |
| | | (m, 1H), 2.21-1.85 (m, 5H). | |
| 262 | N-(5-(4-amino-5-(4- | ¹H NMR (400 MHz, MeOD) δ 8.38 (d, J = 1.3 | 428 |
| | cyclobutoxyphe- | Hz, 1H), 8.09 (d, J = 4.6 Hz, 1H), 8.08-7.98 (m, | |
| | nyl)pyrazolo[5,1- | 2H), 7.39-7.33 (m, 2H), 7.08-6.98 (m, 2H), | |
| | f][1,2,4]triazin-6- | 6.49 (dd, J = 13.8, 5.4 Hz, 2H), 5.87 (dd, J = 8.6, | |
| | yl)pyridin-2- | 3.2 Hz, 1H), 4.78-4.73 (m, 1H), 2.55-2.46 (m, | |
| | yl)acrylamide | 2H), 2.22-2.14 (m, 2H), 1.93-1.74 (m, 2H). | |
| 263 | N-(4-(6-(4- | ¹H NMR (400 MHz, DMSO) δ 10.41 (s, 1H), | 440 |
| | acrylamidophenyl)-4- | 10.22 (s, 1H), 8.13 (s, 1H), 7.74 (d, J = 8.8 Hz, | |
| | aminopyrazolo[5,1- | 2H), 7.60 (d, J = 8.8 Hz, 2H), 7.40 (d, J = 8.8 | |
| | f][1,2,4]triazin-5- | Hz, 2H), 7.34 (d, J = 8.8 Hz, 2H), 6.42 (dd, J = | |
| | yl)phenyl)cyclopropane | 17.4, 10.0 Hz, 1H), 6.25 (dd, J = 16.8, 2.0 Hz, | |
| | carboxamide | 1H), 5.76 (dd, J = 10.0, 1.6 Hz, 1H), 1.87-1.78 | |
| | | (m, 1H), 0.88-0.79 (m, 4H). | |
| 264 | N-(4-(4-amino-5-(4- | ¹H NMR (400 MHz, DMSO) δ 10.21 (s, 1H), | 426 |
| | (pyrrolidin-1- | 8.33 (s, 1H), 8.10 (s, 1H), 7.60 (d, J = 8.8 Hz, | |
| | yl)phenyl)pyrazolo[5,1- | 2H), 7.47 (d, J = 8.8 Hz, 2H), 7.20 (d, J = 8.4 | |
| | f][1,2,4]triazin-6- | Hz, 2H), 6.66 (d, J = 8.4 Hz, 2H), 6.43 (dd, J = | |
| | yl)phenyl)acrylamide | 16.8, 10.0 Hz, 1H), 6.25 (dd, J = 16.8, 2.0 Hz, | |
| | | 1H), 5.76 (dd, J = 10.0, 2.0 Hz, 1H), 5.27 (s, | |
| | | 1H), 3.30 (t, J = 6.4 Hz, 4H), 1.99 (t, J = 6.4 Hz, | |
| | | 4H). | |
| 265 | N-(4-(2-(4- | ¹H NMR (400 MHz, DMSO) δ 10.26 (s, 1H), | 52 |
| | acrylamidophenyl)-4- | 8.86 (s, 1H), 8.18 (s, 1H), 7.99 (d, J = 8.0 Hz, | |
| | amino-7-cyano-1- | 1H), 7.68 (d, J = 8.8 Hz, 2H), 7.29 (d, J = 8.8 | |
| | methyl-1H-pyrrolo[3,2- | Hz, 2H), 6.84 (d, J = 1.6 Hz, 1H), 6.79 (m, 1H), | |
| | c]pyridin-3-yl)-2- | 6.43 (m, 1H), 6.26 (m, 1H), 5.77 (m, 1H), 3.80 | |
| | methoxyphenyl)cyclo- | (s, 3H), 3.67 (s, 3H), 3.45-3.36 (m, 1H), 2.24- | |
| | butanecarboxamide | 2.14 (m, 2H), 2.07 (m, 2H), 1.95-1.76 (m, 2H). | |
| 266 | N-(4-(2-(4- | ¹H NMR (400 MHz, DMSO) δ 10.26 (s, 1H), | 535 |
| | acrylamidophenyl)-4- | 8.98 (s, 1H), 8.18 (s, 1H), 7.97 (d, J = 8.0 Hz, | |
| | amino-7-cyano-1- | 1H), 7.68 (d, J = 8.8 Hz, 2H), 7.29 (d, J = 8.4 | |
| | methyl-1H-pyrrolo[3,2- | Hz, 2H), 6.85 (d, J = 1.6 Hz, 1H), 6.80-6.76 (m, | |
| | c]pyridin-3-yl)-2- | 1H), 6.43 (m, 1H), 6.26 (m, 1H), 5.77 (m, 1H), | |
| | methoxyphenyl)cyclo- | 3.80 (s, 3H), 3.68 (s, 3H), 3.01-2.87 (m, 1H), | |
| | pentanecarboxamide | 1.81 (m, 2H), 1.68 (m, 4H), 1.52 (s, 2H). | |
| 267 | 4-(4-amino-7-cyano-2- | ¹H NMR (400 MHz, DMSO) δ 12.64 (s, 1H), | 529 |
| | (2-fluoro-4-(2- | 10.55 (s, 1H), 8.17 (s, 1H), 8.10 (d, J = 4.3 Hz, | |
| | fluoroacrylamido)phe- | 1H), 7.71-7.66 (m, 1H), 7.63-7.60 (m, 1H), | |
| | nyl)-1H-pyrrolo[3,2- | 7.52-7.48 (m, 1H), 7.33-7.28 (m, 1H), 6.99- | |
| | c]pyridin-3-yl)-N- | 6.96 (m, 1H), 6.90-6.87 (m, 1H), 6.57-5.53 | |
| | cyclopropyl-2- | (m, 3H), 5.48 (dd, J = 15.6, 3.7 Hz, 1H), 3.72 (s, | |
| | methoxybenzamide | 3H), 2.85-2.78 (m, 1H), 0.71-0.66 (m, 2H), | |
| | | 0.55-0.51 (m, 2H). | |
| 268 | 4-(6-(4- | ¹H NMR (400 MHz, DMSO) δ 10.22 (s, 1H), | 495 |
| | acrylamidophenyl)-4- | 8.96 (s, 1H), 8.16 (s, 1H), 7.81 (d, J = 7.8 Hz, | |
| | aminopyrazolo[5,1- | 1H), 7.62 (d, J = 8.8 Hz, 2H), 7.39 (d, J = 8.8 | |
| | f][1,2,4]triazin-5-yl)-N- | Hz, 2H), 7.21 (d, J = 1.2 Hz, 1H), 7.04 (dd, J = | |
| | (1-cyanocyclopropyl)- | 8.0, 1.3 Hz, 1H), 6.43 (dd, J = 17.2, 10.0 Hz, | |
| | 2-methoxybenzamide | 1H), 6.25 (dd, J = 17.2, 2.0 Hz, 1H), 5.76 (dd, | |
| | | J = 10.0, 2.0 Hz, 1H), 3.82 (s, 3H), 1.57 (dd, J = | |
| | | 8.4, 5.6 Hz, 2H), 1.30 (dd, J = 8.4, 5.6 Hz, 2H). | |
| 269 | 4-(6-(4- | ¹H NMR (400 MHz, DMSO) δ 10.23 (s, 1H), | 496 |
| | acrylamidophenyl)-4- | 8.54 (s, 1H), 8.16 (s, 1H), 7.76 (d, J = 7.8 Hz, | |
| | aminopyrazolo[5,1- | 1H), 7.62 (d, J = 8.8 Hz, 2H), 7.40 (d, J = 8.8 | |
| | f][1,2,4]triazin-5-yl)-N- | Hz, 2H), 7.19 (d, J = 1.2 Hz, 1H), 7.02 (dd, J = | |
| | (bicyclo[1.1.1 ]pentan- | 7.8, 1.6 Hz, 1H), 6.43 (dd, J = 17.0, 10.2 Hz, | |
| | 1-yl)-2- | 1H), 6.25 (dd, J = 17.2, 2.0 Hz, 1H), 5.76 (dd, | |
| | methoxybenzamide | J = 10.0, 2.0 Hz, 1H), 3.81 (s, 3H), 2.47 (s, 1H), | |
| | | 2.10 (s, 6H). | |
| 270 | 4-(4-amino-6-(4-(2- | ¹H NMR (400 MHz, DMSO) δ 10.37 (s, 1H), | 514 |
| | fluoroacrylamido)phe- | 8.54 (s, 1H), 8.17 (s, 1H), 7.75 (d, J = 8.0 Hz, | |
| | nyl)pyrazolo[5,1- | 1H), 7.68 (d, J = 8.8 Hz, 2H), 7.41 (d, J = 8.8 | |
| | f][1,2,4]triazin-5-yl)-N- | Hz, 2H), 7.20 (d, J = 0.8 Hz, 1H), 7.02 (dd, J = | |
| | (bicyclo[1.1.1 ]pentan- | 7.8, 1.2 Hz, 1H), 5.71 (dd, J = 47.8, 3.6 Hz, 1H), | |
| | 1-yl)-2- | 5.44 (dd, J = 15.6, 3.6 Hz, 1H), 3.81 (s, 3H), | |
| | methoxybenzamide | 2.47 (s, 1H), 2.10 (s, 6H). | |
| 271 | 4-(4-amino-6-(2-fluoro- | ¹H NMR (400 MHz, DMSO) δ 10.57 (s, 1H), | 542 |
| | 4-(2- | 8.46-8.39 (m, 2H), 8.21 (s, 1H), 7.76-7.56 (m, | |
| | fluoroacrylamido)phe- | 3H), 7.45 (t, J = 8.4 Hz, 1H), 7.09 (d, J = 1.0 Hz, | |
| | nyl)pyrazolo[5,1-f][1,2,4] | 1H), 6.93-6.91 (m, 1H), 6.04 (s, 1H), 5.81- | |
| | triazin-5-yl)-N-(2,2- | 5.68 (m, 1H), 5.51-5.46 (m, 1H), 3.76 (s, 3H), | |
| | difluorocyclopropyl)-2- | 3.52-3.41 (m, 1H), 2.01-1.86 (m, 1H), 1.78- | |
| | methoxybenzamide | 1.63 (m, 1H). | |
| 272 | N-(4-(6-(4- | ¹H NMR (400 MHz, DMSO) δ 10.22 (s, 1H), | 484 |
| | acrylamidophenyl)-4- | 8.99 (s, 1H), 8.21 (d, J = 8.4 Hz, 1H), 8.13 (s, | |
| | aminopyrazolo[5,1- | 1H), 7.61 (d, J = 8.8 Hz, 2H), 7.43 (d, J = 8.8 | |
| | f][1,2,4]triazin-5-yl)-2- | Hz, 2H), 7.08 (d, J = 1.6 Hz, 1H), 6.95 (m, 1H), | |
| | methoxyphenyl)cyclo- | 6.43 (m, 1H), 6.25 (m, 1H), 5.76 (m, 1H), 3.76 | |
| | butanecarboxamide | (s, 3H), 3.53-3.42 (m, 1H), 2.22 (m, 2H), 2.11 | |
| | | (m, 2H), 1.93 (m, 1H), 1.81 (m, 1H). | |
| 273 | N-(4-(4-amino-5-(4-(3- | ¹H NMR (400 MHz, DMSO) δ 10.21 (s, 1H), | 457 |
| | methoxycyclobutoxy)phe- | 8.13 (s, 1H), 7.60 (d, J = 8.7 Hz, 2H), 7.39 (d, | |
| | nyl)pyrazolo[5,1- | J = 8.7 Hz, 2H), 7.32 (d, J = 8.6 Hz, 2H), 7.01- | |
| | f][1,2,4]triazin-6- | 6.93 (m, 2H), 6.42 (dd, J = 17.0, 10.1 Hz, 1H), | |
| | yl)phenyl)acrylamide | 6.25 (dd, J = 17.0, 2.0 Hz, 1H), 5.76 (dd, J = | |
| | | 10.1, 2.0 Hz, 1H), 4.93-4.87 (m, 0.4H), 4.48- | |
| | | 4.41 (m, 0.6H), 4.12-4.07 (m, 0.4H), 3.68-3.61 | |
| | | (m, 0.6H), 3.18 (s, 1H), 3.17 (s, 2H), 2.94-2.84 | |
| | | (m, 1.4H), 2.47-2.41 (m, 1.4H), 2.38-2.32 (m, | |
| | | 0.6H), 1.98-1.92 (m, 1H). | |
| 274 | N-(4-(4-amino-5-(4- | ¹H NMR (400 MHz, DMSO) δ 10.21 (s, 1H), | 453 |
| | (spiro[2.3 ]hexan-5- | 8.12 (s, 1H), 7.60 (d, J = 8.7 Hz, 2H), 7.39 (d, | |
| | yloxy)phenyl)pyrazolo[5,1- | J = 8.7 Hz, 2H), 7.33 (d, J = 8.6 Hz, 2H), 6.99 (d, | |
| | f][1,2,4]triazin-6- | J = 8.7 Hz, 2H), 6.42 (dd, J = 17.0, 10.1 Hz, 1H), | |
| | yl)phenyl)acrylamide | 6.25 (dd, J = 17.0, 2.0 Hz, 1H), 5.76 (dd, J = | |
| | | 10.1, 2.0 Hz, 1H), 4.98 (m, 1H), 2.45-2.30 (m, | |
| | | 4H), 0.55-0.45 (m, 4H). | |
| 275 | N-(4-(4-amino-5-(3- | ¹H NMR (400 MHz, DMSO) δ 10.38 (s, 1H), | 501 |
| | fluoro-4-((4- | 8.53 (d, J = 5.0 Hz, 1H), 8.17 (s, 1H), 7.72 (d, | |
| | methylpyrimidin-2- | J = 8.7 Hz, 2H), 7.51-7.45 (m, 2H), 7.42 (d, J = | |
| | yl)oxy)phenyl)pyrazolo | 8.7 Hz, 2H), 7.29 (d, J = 9.6 Hz, 1H), 7.23 (d, | |
| | [5,1-f][1,2,4]triazin-6- | J = 5.0 Hz, 1H), 5.78 (d, J = 3.7 Hz, 0.5H), 5.66 | |
| | yl)phenyl)-2- | (d, J = 3.7 Hz, 0.5H), 5.44 (dd, J = 15.6, 3.6 Hz, | |
| | fluoroacrylamide | 1H), 2.45 (s, 3H). | |
| 276 | 4-(2-(4-acrylamido-2- | ¹H NMR (400 MHz, DMSO) δ 12.43 (s, 1H), | 573 |
| | methoxyphenyl)-4- | 10.26 (s, 1H), 8.48 (d, J = 6.8 Hz, 1H), 8.16 (s, | |
| | amino-7-cyano-1H- | 1H), 7.63 (d, J = 7.8 Hz, 1H), 7.50 (s, 1H), 7.20- | |
| | pyrrolo[3,2-c]pyridin-3- | 7.07 (m, 2H), 6.94 (s, 1H), 6.87 (d, J = 8.4 Hz, | |
| | yl)-N-(3,3- | 1H), 6.46-6.39(m, 1H), 6.29-6.25 (m, 1H), | |
| | difluorocyclobutyl)-2- | 5.80-5.77 (m, 1H), 4.31-4.17 (m, 1H), 3.73 (s, | |
| | methoxybenzamide | 3H), 3.58 (s, 3H), 3.00-2.85 (m, 2H), 2.82- | |
| | | 2.64 (m, 2H). | |
| 277 | N-(4-(4-amino-7- | ¹H NMR (400 MHz, DMSO) δ 10.29 (s, 1H), | 495 |
| | cyano-3-(3-methoxy-4- | 9.06 (s, 1H), 8.38 (s, 1H), 8.03 (d, J = 8.1 Hz, | |
| | propionamidophenyl)- | 1H), 7.70 (d, J = 8.6 Hz, 2H), 7.29 (d, J = 8.6 | |
| | 1-methyl-1H- | Hz, 2H), 6.85 (d, J = 1.8 Hz, 1H), 6.79 (dd, J = | |
| | pyrrolo[3,2-c]pyridin-2- | 8.2, 1.8 Hz, 1H), 6.43 (dd, J = 17.0, 10.1 Hz, | |
| | yl)phenyl)acrylamide | 1H), 6.27 (dd, J = 17.0, 2.0 Hz, 1H), 5.78 (dd, | |
| | | J = 10.1, 2.0 Hz, 1H), 3.86 (s, 3H), 3.68 (s, 3H), | |
| | | 2.39 (q, J = 7.5 Hz, 2H), 1.05 (t, J = 7.5 Hz, 3H). | |
| 278 | 4-(2-(4- | ¹H NMR (400 MHz, DMSO) δ 10.28 (s, 1H), | 589 |
| | acrylamidophenyl)-4- | 8.48 (d, J = 6.8 Hz, 1H), 8.23 (s, 1H), 7.69 (d, | |
| | amino-7-cyano-1-(2- | J = 8.4 Hz, 2H), 7.58 (d, J = 8.0 Hz, 1H), 7.33 (d, | |
| | fluoroethyl)-1H- | J = 8.4 Hz, 2H), 6.98 (s, 1H), 6.89 (d, J = 8.0 | |
| | pyrrolo[3,2-c]pyridin-3- | Hz, 1H), 6.43 (m, 1H), 6.27 (m, 2H), 5.78 (dd, | |
| | yl)-N-(3,3- | J = 10.0, 1.9 Hz, 1H), 4.66-4.58 (m, 2H), 4.53 (s, | |
| | difluorocyclobutyl)-2- | 2H), 4.22 (m, 1H), 3.72 (s, 4H), 2.94-2.86 (m, | |
| | methoxybenzamide | 2H), 2.74-2.66 (m, 3H). | |
| 279 | 4-(4-amino-6-(4-(2- | ¹H NMR (400 MHz, DMSO) δ 10.37 (s, 1H), | 513 |
| | fluoroacrylamido)phe- | 8.96 (s, 1H), 8.17 (s, 1H), 7.81 (d, J = 8.0 Hz, | |
| | nyl)pyrazolo[5,1- | 1H), 7.68 (d, J = 8.8 Hz, 2H), 7.40 (d, J = 8.8 | |
| | f][1,2,4]triazin-5-yl)-N- | Hz, 2H), 7.22 (d, J = 1.2 Hz, 1H), 7.02 (d, J = | |
| | (1-cyanocyclopropyl)- | 1.2 Hz, 1H), 5.71 (dd, J = 47.6, 3.6 Hz, 1H), 5.44 | |
| | 2-methoxybenzamide | (dd, J = 15.6, 3.6 Hz, 1H), 3.82 (s, 3H), 1.57 (dd, | |
| | | J = 8.4, 5.6 Hz, 2H), 1.30 (dd, J = 8.4, 5.6 Hz, | |
| | | 2H). | |
| 280 | N-(4-(4-amino-5-(4- | ¹H NMR (400 MHz, DMSO) δ 10.21 (s, 1H), | 426 |
| | (cyclobutylamino)phe- | 8.09 (s, 1H), 7.60 (d, J = 8.8 Hz, 2H), 7.46 (d, | |
| | nyl)pyrazolo[5,1- | J = 8.8 Hz, 2H), 7.10 (d, J = 8.4 Hz, 2H), 6.62 (d, | |
| | f][1,2,4]triazin-6- | J = 8.4 Hz, 2H), 6.43 (m, 1H), 6.26 (m, 2H), | |
| | yl)phenyl)acrylamide | 5.76 (m, 1H), 3.96-3.78 (m, 1H), 2.36 (m, 2H), | |
| | | 1.91-1.83 (m, 2H), 1.77-1.69 (m, 2H). | |
| 281 | 4-(2-(4-acrylamido-2- | ¹H NMR (400 MHz, MeOD) δ 8.09 (s, 1H), 7.82 | 561 |
| | fluorophenyl)-4-amino- | (d, J = 8.3 Hz, 1H), 7.70 (dd, J = 12.4, 1.9 Hz, | |
| | 7-cyano-1H- | 1H), 7.29-7.18 (m, 2H), 7.09-6.99 (m, 2H), | |
| | pyrrolo[3,2-c]pyridin-3- | 6.40 (dd, J = 7.4, 3.5 Hz, 2H), 5.80 (dd, J = 7.7, | |
| | yl)-N-(3,3- | 4.1 Hz, 1H), 4.36-4.28 (m, 1H), 3.82 (s, 3H), | |
| | difluorocyclobutyl)-2- | 3.02-2.94 (m, 2H), 2.72-2.62 (m, 2H). | |
| | methoxybenzamide | | |
| 282 | 4-(2-(4- | ¹H NMR (400 MHz, DMSO) δ 10.28 (s, 1H), | 585 |
| | acrylamidophenyl)-4- | 8.46 (d, J = 6.4 Hz, 1H), 8.22 (s, 1H), 7.68 (d, | |
| | amino-7-cyano-1- | J = 8.8 Hz, 2H), 7.56 (d, J = 7.6 Hz, 1H), 7.35 (s, | |
| | isopropyl-1H- | 2H), 6.95 (s, 1H), 6.89 (d, J = 8.0 Hz, 1H), 6.42 | |
| | pyrrolo[3,2-c]pyridin-3- | (dd, J = 16.8, 10.0 Hz, 1H), 6.26 (dd, J = 16.8, | |
| | yl)-N-(3,3- | 2.0 Hz, 1H), 5.78 (dd, J = 10.0, 2.0 Hz, 1H), | |
| | difluorocyclobutyl)-2- | 4.72-5.12 (m, 1H), 4.27-4.15 (m, 1H), 3.70 (s, | |
| | methoxybenzamide | 3H), 2.98-2.82 (m, 2H), 2.78-2.65 (m, 2H), | |
| | | 1.47 (s, 6H). | |
| 283 | N-(4-(2-(4- | ¹H NMR (400 MHz, DMSO) δ 10.27 (s, 1H), | 521 |
| | acrylamidophenyl)-4- | 8.20 (s, 1H), 7.65 (s, 2H), 7.27 (s, 3H), 6.90 (d, | |
| | amino-7-cyano-1- | J = 38.7 Hz, 2H), 6.41 (d, J = 10.1 Hz, 1H), 6.26 | |
| | methyl-1H-pyrrolo[3,2- | (d, J = 16.9 Hz, 3H), 5.78 (d, J = 9.7 Hz, 1H), | |
| | c]pyridin-3-yl)-2- | 3.83 (s, 3H), 3.65 (s, 3H), 3.04 (s, 3H), 1.16 (m, | |
| | methoxyphenyl)-N- | 1H), 0.69 (s, 2H), 0.53 (s, 2H). | |
| | methylcyclopropanecar- | | |
| | boxamide | | |
| 284 | N-(4-(2-(4- | ¹H NMR (400 MHz, MeOD) δ 8.27 (s, 1H), 7.70 | 477 |
| | acrylamidophenyl)-4- | (d, J = 8.8 Hz, 2H), 7.57 (d, J = 8.4 Hz, 2H), | |
| | amino-7-cyano-1- | 7.26 (dd, J = 16.0, 8.4 Hz, 4H), 6.49-6.31 (m, | |
| | methyl-1H-pyrrolo[3,2- | 2H), 5.79 (dd, J = 9.2, 2.8 Hz, 1H), 4.02 (s, 3H), | |
| | c]pyridin-3- | 1.80-1.69 (m, 1H), 0.97-0.90 (m, 2H), 0.90- | |
| | yl)phenyl)cyclopropanecar- | 0.82 (m, 2H). | |
| | boxamide | | |
| 285 | N-(4-(2-(4- | ¹H NMR (400 MHz, DMSO) δ 10.30 (s, 1H), | 557 |
| | acrylamidophenyl)-4- | 9.39 (s, 1H), 8.40 (s, 1H), 8.04 (d, J = 8.4 Hz, | |
| | amino-7-cyano-1- | 1H), 7.70 (d, J = 8.4 Hz, 2H), 7.29 (d, J = 8.4 | |
| | methyl-1H-pyrrolo[3,2- | Hz, 2H), 6.87 (d, J = 1.6 Hz, 1H), 6.80 (dd, J = | |
| | c]pyridin-3-yl)-2- | 8.4, 1.6 Hz, 1H), 6.43 (dd, J = 16.0, 10. Hz, 1H), | |
| | methoxyphenyl)-3,3- | 6.27 (dd, J = 16.0, 2.0 Hz, 1H), 5.78 (dd, J = 10., | |
| | difluorocyclobutane-1- | 2.0 Hz, 1H), 3.86 (s, 4H), 3.69 (s, 3H), 3.37- | |
| | carboxamide | 3.30 (m, 1H), 2.80-2.70 (m, 4H). | |
| 286 | N-(4-(2-(4- | ¹H NMR (400 MHz, DMSO) δ 10.26 (s, 1H), | 521 |
| | acrylamidophenyl)-4- | 8.42 (s, 1H), 8.19 (s, 1H), 7.87 (d, J = 8.1 Hz, | |
| | amino-7-cyano-1- | 1H), 7.68 (d, J = 8.6 Hz, 2H), 7.30 (d, J = 8.6 | |
| | methyl-1H-pyrrolo[3,2- | Hz, 2H), 6.89 (d, J = 1.7 Hz, 1H), 6.80 (dd, J = | |
| | c]pyridin-3-yl)-2- | 8.1, 1.7 Hz, 1H), 6.43 (dd, J = 16.9, 10.1 Hz, | |
| | methoxyphenyl)-1- | 1H), 6.26 (dd, J = 17.0, 2.0 Hz, 1H), 5.77 (dd, | |
| | methylcyclopropane-1- | J = 10.1, 2.0 Hz, 1H), 3.81 (s, 3H), 3.71 (s, 3H), | |
| | carboxamide | 1.39 (s, 3H), 1.06 (q, J = 3.5 Hz, 2H), 0.65 (q, | |
| | | J = 3.7 Hz, 2H). | |
| 287 | N-(4-(2-(4- | ¹H NMR (400 MHz, DMSO) δ 10.27 (s, 1H), | 525 |
| | acrylamidophenyl)-4- | 9.06 (d, J = 3.6 Hz, 1H), 8.19 (s, 1H), 7.86 (d, | |
| | amino-7-cyano-1- | J = 8.0 Hz, 1H), 7.69 (d, J = 8.8 Hz, 2H), 7.31 (d, | |
| | methyl-1H-pyrrolo[3,2- | J = 8.4 Hz, 2H), 6.95 (d, J = 1.6 Hz, 1H), 6.84 | |
| | c]pyridin-3-yl)-2- | (m, 1H), 6.43 (m, 1H), 6.26 (m, 1H), 5.77 (m, | |
| | methoxyphenyl)-1- | 1H), 3.80 (s, 3H), 3.73 (s, 3H), 1.43 (m, 2H), | |
| | fluorocyclopropane-1- | 1.30 (m, 2H). | |
| | carboxamide | | |
| 288 | N-(4-(2-(4- | ¹H NMR (400 MHz, DMSO) δ 10.27 (s, 1H), | 576 |
| | acrylamidophenyl)-4- | 8.76 (s, 1H), 8.19 (s, 1H), 7.72 (d, J = 8.0 Hz, | |
| | amino-7-cyano-1- | 1H), 7.68 (d, J = 8.4 Hz, 2H), 7.30 (d, J = 8.4 | |
| | methyl-1H-pyrrolo[3,2- | Hz, 2H), 6.93 (d, J = 1.6 Hz, 1H), 6.82 (dd, J = | |
| | c]pyridin-3-yl)-2- | 8.0, 1.6 Hz, 1H), 6.43 (dd, J = 17.2, 10.1 Hz, | |
| | methoxyphenyl)-1- | 1H), 6.26 (dd, J = 17.2, 2.0 Hz, 1H), 5.77 (dd, | |
| | (trifluoromethyl)cyclo- | J = 10.0, 2.0 Hz, 1H), 3.80 (s, 3H), 3.70 (s, 3H), | |
| | propane-1-carboxamide | 1.45 (7.6, 5.2 Hz, 2H), 1.34 (dd, J = 7.6, 5.2 Hz, | |
| | | 2H). | |
| 289 | 4-(4-amino-6-(2-fluoro- | ¹H NMR (400 MHz, MeOD) δ 8.15 (s, 1H), 7.95 | 530 |
| | 4-(2- | (d, J = 7.9 Hz, 1H), 7.62 (dd, J = 12.2, 1.8 Hz, | |
| | fluoroacrylamido)phe- | 1H), 7.52-7.43 (m, 2H), 7.06 (dd, J = 11.5, 3.6 | |
| | nyl)pyrazolo[5,1-f][1,2,4] | Hz, 2H), 6.16-6.06 (m, 1H), 5.79-5.29 (m, 1H), | |
| | triazin-5-yl)-N-(2,2- | 3.83 (s, 3H). | |
| | difluoroethyl)-2- | | |
| | methoxybenzamide | | |
| 290 | N-(4-(4-amino-7- | ¹H NMR (400 MHz, DMSO) δ 10.26 (s, 1H), | 494 |
| | cyano-3-(4- | 8.17 (s, 1H), 7.68 (d, J = 8.6 Hz, 2H), 7.29 (d, | |
| | cyclobutoxy-3- | J = 8.6 Hz, 2H), 6.79-6.70 (m, 3H), 6.43 (dd, J = | |
| | methoxyphenyl)-1- | 17.0, 10.1 Hz, 1H), 6.27 (dd, J = 17.0, 1.9 Hz, | |
| | methyl-1H-pyrrolo[3,2- | 1H), 5.78 (dd, J = 10.1, 2.0 Hz, 1H), 4.61 (p, J = | |
| | c]pyridin-2- | 7.1 Hz, 1H), 3.79 (s, 3H), 3.61 (s, 3H), 2.42- | |
| | yl)phenyl)acrylamide | 2.36 (m, 2H), 2.09-1.99 (m, 2H), 1.82-1.53 (m, 2H). | |
| 291 | N-(4-(2-(4- | ¹H NMR (400 MHz, DMSO) δ 10.26 (s, 1H), | 543 |
| | acrylamidophenyl)-4- | 9.73 (s, 1H), 8.18 (d, J = 1.9 Hz, 1H), 7.94 (d, | |
| | amino-7-cyano-1- | J = 8.2 Hz, 1H), 7.68 (d, J = 8.5 Hz, 2H), 7.30 (d, | |
| | methyl-1H-pyrrolo[3,2- | J = 8.5 Hz, 2H), 6.88 (s, 1H), 6.80 (d, J = 8.2 Hz, | |
| | c]pyridin-3-yl)-2- | 1H), 6.43 (dd, J = 16.9, 10.1 Hz, 1H), 6.26 (dd, | |
| | methoxyphenyl)-2,2- | J = 17.0, 1.8 Hz, 1H), 5.77 (dd, J = 10.1, 1.9 Hz, | |
| | difluorocyclopropane- | 1H), 3.80 (s, 3H), 3.71 (s, 3H), 3.18-3.09 (m, | |
| | 1-carboxamide | 1H), 2.00-1.88 (m, 2H). | |
| 292 | N-(4-(4-amino-7- | ¹H NMR (400 MHz, DMSO) δ 10.26 (s, 1H), | 522 |
| | cyano-3-(4-(3- | 8.17 (s, 1H), 8.07 (d, J = 8.2 Hz, 1H), 7.82 (s, | |
| | cyclopropylureido)-3- | 1H), 7.68 (d, J = 8.6 Hz, 2H), 7.29 (d, J = 8.6 | |
| | methoxyphenyl)-1- | Hz, 2H), 7.05 (d, J = 2.8 Hz, 1H), 6.79 (s, 1H), | |
| | methyl-1H-pyrrolo[3,2- | 6.77-6.71 (m, 1H), 6.48-6.38 (m, 1H), 6.30- | |
| | c]pyridin-2- | 6.22 (m, 1H), 5.81-5.73 (m, 1H), 3.80 (s, 3H), | |
| | yl)phenyl)acrylamide | 3.68 (s, 3H), 2.55-2.52 (m, 1H), 0.65-0.60 (m, | |
| | | 2H), 0.39-0.34 (m, 2H). | |
| 293 | 4-(4-amino-6-(4-(2- | ¹H NMR (400 MHz, DMSO) δ 10.37 (s, 1H), | 544 |
| | fluoroacrylamido)phe- | 8.53 (d, J = 9.2 Hz, 1H), 8.17 (s, 1H), 7.70 (dd, | |
| | nyl)pyrazolo[5,1-f][1,2,4] | J = 8.4, 4.4 Hz, 2H), 7.42 (d, J = 8.8 Hz, 2H), 7.24 | |
| | triazin-5-yl)-2-methoxy- | (s, 1H), 7.04 (dd, J = 7.6, 1.2 Hz, 1H), 5.71 (dd, | |
| | N-(1,1,1- | J = 47.6, 3.6 Hz, 1H), 5.44 (dd, J = 15.6, 3.6 Hz, | |
| | trifluoropropan-2- | 1H), 4.87 (dq, J = 15.2, 7.6 Hz, 1H), 3.82 (s, | |
| | yl)benzamide | 3H), 1.36 (dd, J = 6.8, 3.6 Hz, 3H). | |
| 294 | 4-(4-amino-6-(3- | ¹H NMR (400 MHz, DMSO) δ 11.89 (s, 1H), | 587 |
| | ((dimethylamino)methyl)- | 8.71 (t, J = 6.4 Hz, 1H), 8.17 (s, 1H), 8.10 (d, J = | |
| | 4-(2-fluoroacrylamido)phe- | 8.4 Hz, 1H), 7.85 (d, J = 8.0 Hz, 1H), 7.34 (dd, | |
| | nyl)pyrazolo[5,1- | J = 8.4, 2.0 Hz, 1H), 7.30 (d, J = 2.0 Hz, 1H), 7.23 | |
| | f][1,2,4] | (d, J = 1.2 Hz, 1H), 7.04 (dd, J = 8.0, 1.2 Hz, | |
| | triazin-5-yl)-2-methoxy- | 1H), 5.73 (d, J = 3.6 Hz, 0.5H), 5.61 (d, J = 3.6 | |
| | N-(2,2,2-trifluoroethyl) | Hz, 0.5H), 5.45 (d, J = 3.6 Hz, 0.5H), 5.42 (d, | |
| | benzamide | J = 3.6 Hz, 0.5H), 4.15 (tt, J = 9.6, 4.8 Hz, 2H), | |
| | | 3.84 (s, 3H), 3.47 (s, 2H), 2.16 (s, 6H). | |
| 295 | N-(4-(4-amino-5-(4- | ¹H NMR (400 MHz, DMSO) δ 10.21 (s, 1H), | 412 |
| | (azetidin-1- | 8.11 (s, 1H), 7.60 (d, J = 8.7 Hz, 2H), 7.43 (d, | |
| | yl)phenyl)pyrazolo[5,1- | J = 8.7 Hz, 2H), 7.20 (d, J = 8.5 Hz, 2H), 6.53 (d, | |
| | f][1,2,4]triazin-6- | J = 8.5 Hz, 2H), 6.46-6.38 (m, 1H), 6.25 (dd, | |
| | yl)phenyl)acrylamide | J = 17.0, 2.0 Hz, 1H), 5.76 (dd, J = 10.1, 2.0 Hz, | |
| | | 1H), 3.89 (m, 4H), 2.34 (m, 2H). | |
| 296 | N-(4-(4-amino-5-(4-(3- | ¹H NMR (400 MHz, DMSO) δ 10.22 (s, 1H), | 485 |
| | cyclopropylureido)-3- | 8.25 (d, J = 8.3 Hz, 1H), 8.12 (s, 1H), 7.97 (s, | |
| | methoxyphenyl)pyrazolo[5,1- | 1H), 7.61 (d, J = 8.7 Hz, 2H), 7.45 (d, J = 8.7 | |
| | f][1,2,4]triazin-6- | Hz, 2H), 7.13 (d, J = 2.8 Hz, 1H), 7.02 (d, J = | |
| | yl)phenyl)acrylamide | 1.7 Hz, 1H), 6.91 (dd, J = 8.2, 1.8 Hz, 1H), 6.43 | |
| | | (dd, J = 17.0, 10.1 Hz, 1H), 6.25 (dd, J = 17.0, | |
| | | 2.0 Hz, 1H), 5.76 (dd, J = 10.1, 1.9 Hz, 1H), 3.77 | |
| | | (s, 3H), 2.55 (m, 1H), 0.67-0.62 (m, 2H), 0.40 | |
| | | (m, 2H). | |
| 297 | N-(4-(6-(4- | ¹H NMR (400 MHz, DMSO) δ 10.23 (s, 1H), | 485 |
| | acrylamidophenyl)-4- | 8.34 (s, 1H), 8.13 (s, 1H), 8.01 (d, J = 8.2 Hz, | |
| | aminopyrazolo[5,1-f] | 1H), 7.61 (d, J = 8.7 Hz, 2H), 7.44 (d, J = 8.7 | |
| | [1,2,4]triazin-5-yl)-2- | Hz, 2H), 7.30 (s, 1H), 7.06 (d, J = 1.6 Hz, 1H), | |
| | methoxyphenyl)azetidine- | 6.95-6.93 (m, 1H), 6.46-6.39 (m, 1H), 6.27- | |
| | 1-carboxamide | 6.23 (m, 1H), 5.77-5.74 (m, 1H), 5.44 (s, 1H), | |
| | | 3.99 (t, J = 7.6 Hz, 4H), 3.76 (s, 3H), 2.27-2.15 | |
| | | (m, 2H). | |
| 298 | N-(4-(4-amino-7-cyano- | ¹H NMR (400 MHz, DMSO) δ 10.25 (s, 1H), | 463 |
| | 3-(4-(cyclobutylamino) | 8.13 (d, J = 2.6 Hz, 1H), 7.67 (d, J = 8.5 Hz, | |
| | phenyl)-1-methyl-1H- | 2H), 7.27 (d, J = 8.5 Hz, 2H), 6.94 (d, J = 8.4 | |
| | pyrrolo[3,2-c]pyridin-2- | Hz, 2H), 6.52-6.38 (m, 3H), 6.27 (d, J = 16.9 | |
| | yl)phenyl)acrylamide | Hz, 1H), 6.05 (d, J = 6.5 Hz, 1H), 5.77 (d, J = | |
| | | 9.9 Hz, 1H), 3.84-3.72 (m, 4H), 2.36-2.24 (m, | |
| | | 2H), 1.83-1.66 (m, 4H). | |
| 299 | N-(4-(2-(4- | ¹H NMR (400 MHz, DMSO) δ 10.26 (s, 1H), | 522 |
| | acrylamidophenyl)-4- | 8.18 (s, 1H), 7.80 (d, J = 8.1 Hz, 1H), 7.67 (d, | |
| | amino-7-cyano-1- | J = 8.6 Hz, 2H), 7.29 (d, J = 8.6 Hz, 2H), 7.18 (s, | |
| | methyl-1H-pyrrolo[3,2- | 1H), 6.82 (d, J = 1.7 Hz, 1H), 6.79-6.76 (m, | |
| | c]pyridin-3-yl)-2- | 1H), 6.46-6.39 (m, 1H), 6.29-6.24 (m, 1H), | |
| | methoxyphenyl)azetidine- | 5.79-5.76 (m, 1H), 3.94 (t, J = 7.6 Hz, 4H), | |
| | 1-carboxamide | 3.80 (s, 3H), 3.67 (s, 3H), 2.20-2.13 (m, 2H). | |
| 300 | 4-(2-(4-acrylamido-3- | ¹H NMR (400 MHz, DMSO-d₆) δ 10.76 (s, 1H), | 606 |
| | ((dimethylamino)methyl)phe- | 8.60 (t, J = 6.4 Hz, 1H), 8.22 (s, 1H), 8.14 (d, J = | |
| | nyl)-4-amino-7- | 8.3 Hz, 1H), 7.72 (d, J = 7.8 Hz, 1H), 7.33 (dd, | |
| | cyano-1-methyl-1H- | J = 8.3, 2.1 Hz, 1H), 7.09 (d, J = 2.1 Hz, 1H), 6.96- | |
| | pyrrolo[3,2-c]pyridin-3- | 6.87 (m, 2H), 6.32 (dd, J = 17.1, 10.1 Hz, 1H), | |
| | yl)-2-methoxy-N- | 6.20 (dd, J = 17.0, 1.7 Hz, 1H), 5.80 (dd, J = | |
| | (2,2,2-trifluoroethyl) | 10.1, 1.7 Hz, 1H), 4.20-4.01 (m, 2H), 3.84 (s, | |
| | benzamide | 2H), 3.72 (s, 3H), 3.42 (s, 2H), 2.07 (s, 5H). | |
| 301 | N-(4-(4-amino-7- | ¹H NMR (400 MHz, DMSO) δ 10.42 (s, 1H), | 520 |
| | cyano-1-methyl-3-(4- | 8.46 (d, J = 5.0 Hz, 1H), 8.19 (d, J = 5.5 Hz, | |
| | ((4-methylpyrimidin-2- | 2H), 7.78 (d, J = 8.6 Hz, 2H), 7.35-7.30 (m, | |
| | yl)oxy)phenyl)-1H- | 4H), 7.20-7.14 (m, 3H), 5.99 (s, 1H), 5.79- | |
| | pyrrolo[3,2-c]pyridin-2- | 5.66 (m, 1H), 5.48-5.43 (m, 1H), 3.81 (s, 3H), | |
| | yl)phenyl)-2- | 2.40 (s, 3H). | |
| | fluoroacrylamide | | |
| 302 | 4-(4-amino-7-cyano-2- | ¹H NMR (400 MHz, DMSO) δ 12.99-12.51 (m, | 553 |
| | (4-(2-fluoroacrylamido) | 1H), 10.36 (s, 1H), 8.73 (t, J = 6.4 Hz, 1H), 8.14 | |
| | phenyl)-1H-pyrrolo | (s, 1H), 7.80 (d, J = 7.9 Hz, 1H), 7.67 (d, J = 8.8 | |
| | [3,2-c] pyridin-3-yl)-2- | Hz, 2H), 7.35 (d, J = 8.7 Hz, 2H), 7.14 (d, J = | |
| | methoxy-N-(2,2,2-tri- | 1.1 Hz, 1H), 6.99 (dd, J = 7.9, 1.3 Hz, 1H), 6.20 | |
| | fluoroethyl)benzamide | (br s, 2H), 5.71 (dd, J = 47.7, 3.7 Hz, 1H), 5.44 | |
| | | (dd, J = 15.6, 3.7 Hz, 1H), 4.18-4.08 (m, 2H), | |
| | | 3.82 (s, 3H). | |
| 303 | 4-(4-amino-7-cyano-2- | ¹H NMR (400 MHz, DMSO) δ 12.50 (s, 1H), | 579 |
| | (4-(2-fluoroacrylamido) | 10.36 (s, 1H), 8.77 (s, 1H), 8.15 (s, 1H), 7.66 (d, | |
| | phenyl)-1H-pyrrolo | J = 5.2 Hz, 3H), 7.35 (d, J = 8.1 Hz, 2H), 7.10 (s, | |
| | [3,2-c]pyridin-3-yl)-2- | 1H), 6.97 (d, J = 7.6 Hz, 1H), 5.71 (d, J = 45.6 | |
| | methoxy-N-(1- | Hz, 1H), 5.44 (d, J = 14.9 Hz, 1H), 3.79 (s, 3H), | |
| | (trifluoromethyl)cyclo- | 1.32 (m, 2H), 1.18 (m, 2H). | |
| | propyl)benzamide | | |
| 304 | 4-(2-(4- | ¹H NMR (400 MHz, DMSO) δ 10.28 (s, 1H), | 601 |
| | acrylamidophenyl)-4- | 8.47 (d, J = 6.8 Hz, 1H), 8.21 (s, 1H), 7.69 (d, | |
| | amino-7-cyano-1-(2- | J = 8.5 Hz, 2H), 7.58 (d, J = 7.8 Hz, 1H), 7.34 (d, | |
| | methoxyethyl)-1H- | J = 8.5 Hz, 2H), 6.94 (s, 1H), 6.89 (d, J = 7.8 Hz, | |
| | pyrrolo[3,2-c]pyridin-3- | 1H), 6.43 (dd, J = 17.0, 10.1 Hz, 1H), 6.27 (dd, | |
| | yl)-N-(3,3- | J = 17.0, 1.9 Hz, 1H), 5.78 (dd, J = 10.1, 1.9 Hz, | |
| | difluorocyclobutyl)-2- | 1H), 4.40 (t, J = 5.6 Hz, 2H), 3.71 (s, 3H), 3.51 | |
| | methoxybenzamide | (t, J = 5.8 Hz, 2H), 3.07 (s, 3H), 2.95-2.86 (m, | |
| | | 2H), 2.77-2.64 (m, 2H). | |
| 305 | 4-(2-(4- | ¹H NMR (400 MHz, DMSO) δ 10.28 (s, 1H), | 581 |
| | acrylamidophenyl)-4- | 8.64 (t, J = 6.4 Hz, 1H), 8.23 (s, 1H), 7.68 (m, | |
| | amino-7-cyano-1-(2- | 3H), 7.33 (d, J = 8.8 Hz, 2H), 7.01 (d, J = 1.2 | |
| | fluoroethyl)-1H- | Hz, 1H), 6.90 (m, 1H), 6.43 (m, 1H), 6.27 (m, | |
| | pyrrolo[3,2-c]pyridin-3- | 1H), 5.78 (m, 1H), 4.60 (m, 4H), 4.12-4.03 (m, | |
| | yl)-2-methoxy-N- | 2H), 3.74 (s, 3H). | |
| | (2,2,2-trifluoroethyl) | | |
| | benzamide | | |
| 306 | 4-(2-(4- | ¹H NMR (400 MHz, DMSO) δ 10.28 (s, 1H), | 575 |
| | acrylamidophenyl)-4- | 8.68 (s, 1H), 8.21 (s, 1H), 7.69 (d, J = 8.4 Hz, | |
| | amino-7-cyano-1- | 2H), 7.56 (d, J = 8 Hz, 1H), 7.30 (d, J = 8.4 Hz, | |
| | methyl-1H-pyrrolo[3,2- | 2H), 6.95 (s, 1H), 6.87 (d, J = 7.6 Hz, 1H), 6.43 | |
| | c]pyridin-3-yl)-2- | (m, 1H), 6.27 (m, 1H), 5.78 (m, 1H), 3.80 (s, | |
| | methoxy-N-(1- | 3H), 3.71 (s, 3H), 1.24 (m, 2H), 1.13 (m, 2H). | |
| | (trifluoromethyl)cyclo- | | |
| | propyl)benzamide | | |
| 307 | 4-(6-(4- | ¹H NMR (400 MHz, DMSO) δ 10.23 (s, 1H), | 538 |
| | acrylamidophenyl)-4- | 8.77 (s, 1H), 8.16 (s, 1H), 7.71 (d, J = 8 Hz, 1H), | |
| | aminopyrazolo[5,1-f] | 7.62 (d, J = 8.4 Hz, 2H), 7.40 (d, J = 8.8 Hz, | |
| | [1,2,4]triazin-5-yl)-2- | 2H), 7.21 (s, 1H), 7.02 (m, 1H), 6.43 (m, 1H), | |
| | methoxy-N-(1- | 6.25 (m, 1H), 5.76 (m, 1H), 3.81 (s, 3H), 1.33 | |
| | (trifluoromethyl)cyclo- | (m, 2H), 1.19 (m, 2H). | |
| | propyl)benzamide | | |
| 308 | 4-(4-amino-6-(4-(2- | ¹H NMR (400 MHz, DMSO) δ 10.37 (s, 1H), | 556 |
| | fluoroacrylamido)phe- | 8.77 (s, 1H), 8.17 (s, 1H), 7.73-7.66 (m, 3H), | |
| | nyl)pyrazolo[5,1-f][1,2,4] | 7.41 (d, J = 8.4 Hz, 2H), 7.22 (s, 1H), 7.02 (d, | |
| | triazin-5-yl)-2-methoxy- | J = 7.6 Hz, 1H), 5.77-5.65 (m, 1H), 5.44 (m, 1H), | |
| | N-(1-(trifluoromethyl) | 3.81 (s, 3H), 1.33 (m, 2H), 1.19 (m, 2H). | |
| | cyclopropyl)benzamide | | |
| 309 | 4-(4-amino-6-(4-(2- | ¹H NMR (400 MHz, DMSO) δ 10.37 (s, 1H), | 570 |
| | fluoroacrylamido)phe- | 8.41 (t, J = 6.0 Hz, 2H), 8.17 (s, 1H), 7.80 (d, J = | |
| | nyl)pyrazolo[5,1-f][1,2,4] | 7.8 Hz, 1H), 7.68 (d, J = 8.8 Hz, 2H), 7.41 (d, | |
| | triazin-5-yl)-2-methoxy- | J = 8.7 Hz, 2H), 7.23 (d, J = 1.1 Hz, 1H), 7.05- | |
| | N-((1-(trifluoromethyl) | 7.02 (m, 1H), 5.78-5.65 (m, 2H), 5.46-5.41 | |
| | cyclopropyl)methyl)benzamide | (m, 1H), 3.83 (s, 3H), 3.65 (d, J = 6.1 Hz, 2H), | |
| | | 0.96 (d, J = 6.0 Hz, 4H). | |
| 310 | 4-(4-amino-6-(4-(2- | ¹H NMR (400 MHz, DMSO) δ 10.36 (s, 1H), | 544 |
| | fluoroacrylamido)phe- | 8.49 (t, J = 5.8 Hz, 1H), 8.17 (s, 1H), 7.87 (d, J = | |
| | nyl)pyrazolo[5,1-f][1,2,4] | 7.9 Hz, 1H), 7.68 (d, J = 8.5 Hz, 2H), 7.40 (d, | |
| | triazin-5-yl)-2-methoxy- | J = 8.5 Hz, 2H), 7.22 (s, 1H), 7.04 (d, J = 7.9 Hz, | |
| | N-(3,3,3- | 1H), 5.71 (dd, J = 47.6, 3.5 Hz, 2H), 5.44 (dd, | |
| | trifluoropropyl) | J = 15.5, 3.6 Hz, 1H), 3.83 (s, 3H), 3.56 (dd, J = | |
| | benzamide | 13.1, 6.6 Hz, 2H), 2.65-2.53 (m, 2H). | |
| 311 | 4-(6-(4-acrylamido-3- | ¹H NMR (400 MHz, DMSO) δ 9.94 (s, 1H), 9.42 | 569 |
| | ((dimethylamino)methl)phe- | (s, 1H), 8.72 (t, J = 6.4 Hz, 1H), 8.20 (s, 1H), | |
| | nyl)-4-amino | 7.86 (d, J = 6.0 Hz, 1H), 7.50 (d, J = 8.4 Hz, | |
| | pyrazolo[5,1-f] [1,2,4] | 1H), 7.34 (dd, J = 8.4, 1.9 Hz, 1H), 7.27 (s, 1H), | |
| | triazin-5-yl)-2-methoxy- | 7.05 (dd, J = 8.0, 1.2 Hz, 1H), 6.51 (dd, J = 17.2, | |
| | N-(2,2,2-trifluoroethyl) | 10.0 Hz, 1H), 6.28 (dd, J = 17.2, 2.0 Hz, 1H), | |
| | benzamide | 5.84 (dd, J = 10.0, 2.0 Hz, 1H), 4.30 (d, J = 5.2 | |
| | | Hz, 2H), 4.15 (dd, J = 9.6, 6.4 Hz, 2H), 3.86 (s, | |
| | | 3H), 2.67 (d, J = 4.8 Hz, 6H). | |
| 312 | 4-(6-(4-acrylamido-2- | ¹H NMR (400 MHz, MeOD) δ 8.20 (s, 1H), 8.18 | 569 |
| | ((dimethylamino)methyl)phe- | (d, J = 2.0 Hz, 1H), 7.98 (d, J = 8.0 Hz, 1H), | |
| | nyl)-4-amino- | 7.39 (dd, J = 8.4, 2.0 Hz, 1H), 7.16 (d, J = 8.8 | |
| | pyrazolo[5,1-f][1,2,4] | Hz, 2H), 7.08 (dd, J = 8.0, 1.2 Hz, 1H), 6.43- | |
| | triazin-5-yl)-2-methoxy- | 6.39 (m, 2H), 5.82 (dd, J = 7.6, 4.0 Hz, 1H), 4.37 | |
| | N-(2,2,2-trifluoroethyl) | (s, 2H), 4.16 (q, J = 9.2 Hz, 2H), 3.88 (s, 3H), | |
| | benzamide | 3.03 (s, 6H). | |
| 313 | N-(4-(4-amino-5-(3- | ¹H NMR (400 MHz, DMSO) δ 10.22 (s, 1H), | 456 |
| | methoxy-4-(pyrrolidin- | 8.11 (s, 1H), 7.61 (d, J = 8.4 Hz, 2H), 7.48 (d, | |
| | 1-yl)phenyl)pyrazolo | J = 8.0 Hz, 2H), 6.91 (s, 1H), 6.86 (d, J = 8.0 Hz, | |
| | [5,1-f][1,2,4]triazin-6- | 1H), 6.74 (d, J = 7.6 Hz, 1H), 6.43 (m, 1H), 6.25 | |
| | yl)phenyl)acrylamide | (d, J = 16.8 Hz, 1H), 5.76 (d, J = 9.6 Hz, 1H), | |
| | | 3.67 (s, 3H), 3.31-3.27 (m, 4H), 1.88 (s, 4H). | |
| 314 | N-(4-(4-amino-5-(4- | ¹H NMR (400 MHz, DMSO) δ 10.21 (s, 1H), | 456 |
| | (cyclobutylamino)-3- | 8.31 (s, 1H), 8.11 (s, 1H), 7.61 (d, J = 8.8 Hz, | |
| | methoxyphenyl)pyrazolo[5,1- | 2H), 7.47 (d, J = 8.8 Hz, 2H), 6.86 (d, J = 1.6 | |
| | f][1,2,4]triazin-6- | Hz, 1H), 6.82 (dd, J = 8.0, 1.6 Hz, 1H), 6.59 (d, | |
| | yl)phenyl)acrylamide | J = 8.0 Hz, 1H), 6.43 (dd, J = 17.2, 10.0 Hz, 1H), | |
| | | 6.25 (dd, J = 17.2, 2.0 Hz, 1H), 5.76 (dd, J = | |
| | | 10.0, 2.0 Hz, 1H), 3.92 (dt, J = 15.2, 7.6 Hz, | |
| | | 1H), 3.72 (s, 3H), 2.39-2.26 (m, 2H), 2.06- | |
| | | 1.87 (m, 2H), 1.81-1.65 (m, 2H). | |
| 315 | N-(4-(4-amino-7- | ¹H NMR (400 MHz, DMSO) δ 10.27 (s, 1H), | 463 |
| | cyano-1-methyl-3-(4- | 8.31 (s, 1H), 7.68 (d, J = 8.4 Hz, 2H), 7.27 (d, | |
| | (pyrrolidin-1- | J = 8.4 Hz, 2H), 7.04 (d, J = 8.4 Hz, 2H), 6.52 (d, | |
| | yl)phenyl)-1H- | J = 8.4 Hz, 2H), 6.43 (dd, J = 17.2, 10.0 Hz, 1H), | |
| | pyrrolo[3,2-c]pyridin-2- | 6.26 (dd, J = 17.2, 2.0 Hz, 1H), 5.78 (dd, J = | |
| | yl)phenyl)acrylamide | 10.0, 2.0 Hz, 1H), 3.84 (s, 3H), 3.22 (t, J = 6.4 | |
| | | Hz, 4H), 1.94 (t, J = 6.4 Hz, 4H). | |
| 316 | N-(4-(4-amino-7- | ¹H NMR (400 MHz, DMSO) δ 10.27 (s, 1H), | 493 |
| | cyano-3-(3-methoxy-4- | 8.15 (s, 1H), 7.68 (d, J = 8.0 Hz, 2H), 7.30 (d, | |
| | (pyrrolidin-1- | J = 8.4 Hz, 2H), 6.70 (d, J = 8.8 Hz, 2H), 6.61 (d, | |
| | yl)phenyl)-1-methyl- | J = 8.4 Hz, 1H), 6.43 (m, 1H), 6.26 (d, J = 16.4 | |
| | 1H-pyrrolo[3,2- | Hz, 1H), 5.77 (d, J = 10.0 Hz, 1H), 3.79 (s, 3H), | |
| | c]pyridin-2- | 3.59 (s, 3H), 3.25 (s, 4H), 1.82 (s, 4H). | |
| | yl)phenyl)acrylamide | | |
| 317 | N-(4-(4-amino-7- | ¹H NMR (400 MHz, DMSO) δ 10.26 (s, 1H), | 493 |
| | cyano-3-(4- | 8.14 (s, 1H), 7.67 (d, J = 8.6 Hz, 2H), 7.29 (d, | |
| | (cyclobutylamino)-3- | J = 8.6 Hz, 2H), 6.65 (d, J = 7.6 Hz, 2H), 6.48- | |
| | methoxyphenyl)-1- | 6.39 (m, 2H), 6.26 (dd, J = 17.0, 1.9 Hz, 1H), | |
| | methyl-1H-pyrrolo[3,2- | 5.77 (dd, J = 10.1, 1.9 Hz, 1H), 5.02 (d, J = 6.9 | |
| | c]pyridin-2- | Hz, 1H), 3.88-3.78 (m, 1H), 3.78 (s, 3H), 3.63 | |
| | yl)phenyl)acrylamide | (s, 3H), 1.95-1.82 (m, 2H), 1.68 (dt, J = 17.9, | |
| | | 7.4 Hz, 2H), 1.26-1.06 (m, 2H). | |
| 318 | 4-(2-(4- | ¹H NMR (400 MHz, DMSO) δ 10.27 (s, 1H), | 593 |
| | acrylamidophenyl)-4- | 8.63 (t, J = 6.4 Hz, 1H), 8.22 (s, 1H), 7.71-7.65 | |
| | amino-7-cyano-1-(2- | (m, 3H), 7.33 (d, J = 8.6 Hz, 2H), 6.98 (d, J = 1.2 | |
| | methoxyethyl)-1H- | Hz, 1H), 6.90 (dd, J = 7.9, 1.4 Hz, 1H), 6.43 (dd, | |
| | pyrrolo[3,2-c]pyridin-3- | J = 17.0, 10.1 Hz, 1H), 6.27 (dd, J = 17.0, 2.0 | |
| | yl)-2-methoxy-N- | Hz, 1H), 5.78 (dd, J = 10.1, 2.0 Hz, 1H), 4.41 (t, | |
| | (2,2,2-trifluoroethyl) | J = 5.7 Hz, 2H), 4.12-4.03 (m, 2H), 3.73 (s, | |
| | benzamide | 3H), 3.51 (t, J = 5.7 Hz, 2H), 3.08 (s, 3H). | |
| 319 | 4-(4-amino-7-cyano-2- | ¹H NMR (400 MHz, DMSO) δ 12.75 (br s, 1H), | 561 |
| | (4-(2- | 12.54 (s, 1H), 10.36 (s, 1H), 8.57 (d, J = 6.7 Hz, | |
| | fluoroacrylamido)phe- | 1H), 8.16 (s, 1H), 8.13 (s, 1H), 7.83-7.56 (m, | |
| | nyl)-1H-pyrrolo[3,2- | 3H), 7.35 (d, J = 8.5 Hz, 2H), 7.10 (s, 1H), 6.98 | |
| | c]pyridin-3-yl)-N-(3,3- | (d, J = 7.8 Hz, 1H), 5.71 (dd, J = 47.6, 3.5 Hz, | |
| | difluorocyclobutyl)-2- | 1H), 5.44 (dd, J = 15.6, 3.5 Hz, 1H), 4.38-4.17 | |
| | methoxybenzamide | (m, 1H), 3.79 (s, 3H), 3.04-2.87 (m, 2H), 2.83- | |
| | | 2.66 (m, 2H). | |
| 320 | 4-(4-amino-7-cyano-2- | ¹H NMR (400 MHz, DMSO) δ 12.50 (s, 1H), | 547 |
| | (4-(2- | 10.35 (s, 1H), 8.46 (s, 1H), 8.14 (s, 1H), 7.73 (d, | |
| | fluoroacrylamido)phe- | J = 7.8 Hz, 1H), 7.66 (d, J = 8.6 Hz, 2H), 7.35 | |
| | nyl)-1H-pyrrolo[3,2- | (d, J = 8.7 Hz, 2H), 7.11 (s, 1H), 6.99 (d, J = 8.0 | |
| | c]pyridin-3-yl)-N-(2,2- | Hz, 1H), 5.77 (d, J = 3.6 Hz, 0.5H), 5.65 (d, J = | |
| | difluorocyclopropyl)-2- | 3.5 Hz, 0.5H), 5.44 (dd, J = 15.6, 3.6 Hz, 1H), | |
| | methoxybenzamide | 3.79 (s, 3H), 3.47 (m, 1H), 1.95 (m, 1H), 1.71 | |
| | | (m, 1H). | |
| 321 | 4-(4-amino-7-cyano-2- | ¹H NMR (400 MHz, DMSO) δ 12.48 (s, 1H), | 525 |
| | (4-(2- | 10.35 (s, 1H), 8.31 (s, 1H), 8.16-8.13 (m, 1H), | |
| | fluoroacrylamido)phe- | 7.73 (d, J = 7.8 Hz, 1H), 7.66 (d, J = 8.7 Hz, | |
| | ny1)-1H-pyrrolo[3,2- | 2H), 7.34 (d, J = 8.7 Hz, 2H), 7.07 (s, 1H), 6.95 | |
| | c]pyridin-3-yl)-2- | (d, J = 7.8 Hz, 1H), 5.77 (d, J = 3.7 Hz, 0.5H), | |
| | methoxy-N-(1- | 5.65 (d, J = 3.7 Hz, 0.5H), 5.44 (dd, J = 15.6, 3.7 | |
| | methylcyclopropyl)benzamide | Hz, 1H), 3.78 (s, 3H), 1.39 (s, 3H), 0.74 (m, 2H), | |
| | | 0.61 (m, 2H). | |
| 322 | N-(4-(4-amino-5-(1- | ¹H NMR (400 MHz, DMSO) δ 10.18 (s, 1H), | 410 |
| | methyl-1H-indol-5- | 8.16-8.09 (m, 1H), 7.57 (dd, J = 26.3, 9.1 Hz, | |
| | yl)pyrazolo[5,1- | 4H), 7.47-7.39 (m, 3H), 7.18 (d, J = 8.3 Hz, | |
| | f][1,2,4]triazin-6- | 1H), 6.49 (s, 1H), 6.40 (dd, J = 16.5, 10.3 Hz, | |
| | yl)phenyl)acrylamide | 1H), 6.23 (d, J = 16.9 Hz, 1H), 5.74 (d, J = 10.2 | |
| | | Hz, 1H), 3.87 (s, 3H). | |
| 323 | N-(4-(4-amino-5-(1- | ¹H NMR (400 MHz, DMSO) δ 10.31 (s, 1H), | 428 |
| | methyl-1H-indol-5- | 8.13 (s, 1H), 7.65-7.57 (m, 4H), 7.44 (dd, J = | |
| | yl)pyrazolo[5,1- | 13.1, 5.9 Hz, 3H), 7.19 (dd, J = 8.4, 1.5 Hz, 1H), | |
| | f][1,2,4]triazin-6- | 6.49 (d, J = 3.0 Hz, 1H), 5.74 (d, J = 3.6 Hz, | |
| | yl)phenyl)-2- | 0.5H), 5.62 (d, J = 3.6 Hz, 0.5H), 5.42 (dd, J = | |
| | fluoroacrylamide | 15.6, 3.7 Hz, 1H), 3.87 (s, 3H). | |
| 324 | 4-(4-amino-6-(4-(2- | ¹H NMR (400 MHz, DMSO) δ 10.36 (s, 1H), | 500 |
| | fluoroacrylamido)phe- | 9.22 (m, 1H), 8.17 (s, 1H), 8.01 (d, J = 8.3 Hz, | |
| | nyl)pyrazolo[5,1- | 2H), 7.67 (d, J = 8.8 Hz, 2H), 7.53 (d, J = 8.3 | |
| | f][1,2,4]triazin-5-yl)-N- | Hz, 2H), 7.35 (d, J = 8.8 Hz, 2H), 5.76 (d, J = | |
| | (2,2,2- | 3.7 Hz, 0.5H), 5.65 (d, J = 3.6 Hz, 0.5H), 5.43 | |
| | trifluoroethyl)benzamide | (dd, J = 15.6, 3.7 Hz, 1H), 4.13 (m, 2H). | |
| 325 | N-(4-(4-amino-5-(4-((4- | ¹H NMR (400 MHz, DMSO) δ 10.37 (s, 1H), | 483 |
| | methylpyrimidin-2- | 8.51 (d, J = 4.8 Hz, 1H), 8.17 (s, 1H), 7.70 (d, | |
| | yl)oxy)phenyl)pyrazolo | J = 8.8 Hz, 2H), 7.49 (d, J = 8.4 Hz, 2H), 7.44 (d, | |
| | [5,1-f][1,2,4]triazin-6- | J = 8.8 Hz, 2H), 7.34 (d, J = 8.4 Hz, 2H), 7.19 | |
| | yl)phenyl)-2- | (d, J = 5.2 Hz, 1H), 5.71 (m, 1H), 5.44 (m, 1H), | |
| | fluoroacrylamide | 2.44 (s, 3H). | |
| 326 | N-(4-(4-amino-7- | ¹H NMR (400 MHz, MeOD) δ 8.07 (s, 1H), 7.59 | 447 |
| | cyano-1-methyl-3-(1- | (d, J = 8.6 Hz, 2H), 7.47 (s, 1H), 7.37 (d, J = 8.4 | |
| | methyl-1H-indol-5-yl)- | Hz, 1H), 7.27 (d, J = 8.6 Hz, 2H), 7.19 (d, J = | |
| | 1H-pyrrolo[3,2- | 3.2 Hz, 1H), 7.10-7.02 (m, 1H), 6.42-6.32 (m, | |
| | c]pyridin-2- | 3H), 5.75 (dd, J = 9.3, 2.5 Hz, 1H), 3.94 (s, 3H), | |
| | yl)phenyl)acrylamide | 3.80 (s, 3H). | |
| 327 | N-(4-(4-amino-3- | ¹H NMR (400 MHz, DMSO) δ 10.28 (s, 1H), | 450 |
| | (benzo[b]thiophen-2- | 8.23 (s, 1H), 7.87 (dd, J = 18.8, 7.8 Hz, 2H), | |
| | yl)-7-cyano-1-methyl- | 7.69 (d, J = 8.5 Hz, 2H), 7.43-7.32 (m, 5H), | |
| | 1H-pyrrolo[3,2- | 6.45-6.38 (m, 1H), 6.25 (d, J = 16.8 Hz, 1H), | |
| | c]pyridin-2- | 5.77 (d, J = 10.0 Hz, 1H), 3.82 (s, 3H). | |
| | yl)phenyl)acrylamide | | |
| 328 | 4-(2-(4- | ¹H NMR (400 MHz, DMSO) δ 10.29 (s, 1H), | 577 |
| | acrylamidophenyl)-4- | 8.62 (t, J = 6.4 Hz, 1H), 8.30 (s, 1H), 7.72-7.63 | |
| | amino-7-cyano-1- | (m, 3H), 7.35 (s, 2H), 6.99 (s, 1H), 6.91 (d, J = | |
| | isopropyl-1H- | 9.2 Hz, 1H), 6.43 (m, 1H), 6.27 (m, 1H), 5.82- | |
| | pyrrolo[3,2-c]pyridin-3- | 5.74 (m, 1H), 4.08 (d, J = 7.4 Hz, 2H), 3.73 (s, | |
| | yl)-2-methoxy-N- | 3H), 3.60 (s, 1H), 1.47 (s, 6H). | |
| | (2,2,2- | | |
| | trifluoroethyl)benzamide | | |
| 329 | 4-(2-(4-acryl- | ¹H NMR (400 MHz, DMSO) δ 10.30 (s, 1H), | 617 |
| | amidophenyl)-4-amino- | 8.65 (s, 1H), 8.32 (s, 1H), 7.69 (m, 3H), 7.31 (d, | |
| | 7-cyano-1-(2,2,2-tri- | J = 8.3 Hz, 2H), 6.98 (d, J = 63.0 Hz, 2H), 6.43 | |
| | fluoroethyl)-1H- | (m, 1H), 6.30-6.24 (m, 1H), 5.78 (d, J = 12.0 | |
| | pyrrolo[3,2-c]pyridin-3- | Hz, 1H), 5.13 (s, 2H), 4.10-4.02 (m, 2H), 3.76 | |
| | yl)-2-methoxy-N- | (s, 3H). | |
| | (2,2,2-trifluoroethyl) | | |
| | benzamide | | |
| 330 | 4-(4-amino-6-(4-(2- | ¹H NMR (400 MHz, DMSO) δ 10.37 (s, 1H), | 532 |
| | fluoroacrylamido)phe- | 8.71 (s, 1H), 8.17 (s, 1H), 7.74 (d, J = 7.6 Hz, | |
| | nyl)pyrazolo[5,1-f][1,2,4] | 1H), 7.68 (d, J = 8.8 Hz, 2H), 7.41 (d, J = 8.8 | |
| | triazin-5-yl)-N-(3- | Hz, 2H), 7.21 (d, J = 1.2 Hz, 1H), 7.02 (dd, J = | |
| | fluorobicyclo[1.1.1]pentan- | 7.6, 1.2 Hz, 1H), 5.71 (dd, J = 47.6, 3.6 Hz, 1H), | |
| | 1-yl)-2-methoxybenzamide | 5.44 (dd, J = 15.6, 3.6 Hz, 1H), 3.80 (s, 3H), | |
| | | 2.43 (d, J = 2.4 Hz, 6H). | |
| 331 | N-(4-(4-amino-5- | ¹H NMR (400 MHz, DMSO) δ 10.36 (s, 1H), | 431 |
| | (benzo[b]thiophen-2- | 8.21 (s, 1H), 8.01 (d, J = 6.9 Hz, 1H), 7.92 (d, | |
| | yl)pyrazolo[5,1- | J = 6.6 Hz, 1H), 7.67 (s, 2H), 7.62 (s, 1H), 7.53- | |
| | f][1,2,4]triazin-6- | 7.41 (m, 4H), 5.76 (d, J = 3.6 Hz, 0.5H), 5.64 (d, | |
| | yl)phenyl)-2- | J = 3.6 Hz, 0.5H), 5.43 (dd, J = 15.6, 3.6 Hz, | |
| | fluoroacrylamide | 1H). | |
| 332 | N-(4-(4-amino-5- | ¹H NMR (400 MHz, DMSO) δ 10.23 (s, 1H), | 413 |
| | (benzo[b]thiophen-2- | 8.20 (s, 1H), 8.04-7.89 (m, 2H), 7.68-7.58 (m, | |
| | yl)pyrazolo[5,1- | 3H), 7.54-7.37 (m, 4H), 6.41 (dd, J = 16.9, 10.1 | |
| | f][1,2,4]triazin-6- | Hz, 1H), 6.24 (dd, J = 17.0, 1.9 Hz, 1H), 5.75 | |
| | yl)phenyl)acrylamide | (dd, J = 10.1, 1.9 Hz, 1H). | |
| 333 | 4-(4-amino-6-(4-(2- | ¹H NMR (400 MHz, DMSO) δ 10.35 (s, 1H), | 552 |
| | fluoroacrylamido)phe- | 8.41-8.38 (m, 1H), 8.16 (s, 1H), 7.77 (d, J = 8.0 | |
| | nyl)pyrazolo[5,1-f][1,2,4] | Hz, 1H), 7.68 (d, J = 8.8 Hz, 2H), 7.42 (d, J = 8.8 | |
| | triazin-5-yl)-N-((3,3- | Hz, 2H), 7.20 (s, 1H), 7.04-7.02 (m, 1H), 5.77- | |
| | difluorocyclobutyl)methyl)- | 5.64 (m, 1H), 5.45-5.41(m, 1H), 3.81 (s, 3H), | |
| | 2-methoxybenzamide | 3.45-3.42 (m, 2H), 2.66-2.60 (m, 2H), 2.43- | |
| | | 2.33 (m, 3H). | |
| 334 | 4-(4-amino-6-(4-(2- | ¹H NMR (400 MHz, DMSO) δ 10.38 (s, 1H), | 518 |
| | fluoroacrylamido)phe- | 9.00 (s, 1H), 8.18 (s, 1H), 7.72 (m, 3H), 7.39 (m, | |
| | nyl)pyrazolo[5,1-f][1,2,4] | 3H), 7.29 (m, 1H), 5.71 (m, 1H), 5.44 (m, 1H), | |
| | triazin-5-yl)-2-fluoro- | 4.17-4.07 (m, 2H). | |
| | N-(2,2,2-trifluoroethyl) | | |
| | benzamide | | |
| 335 | N-(4-(4-amino-5-(3- | ¹H NMR (400 MHz, DMSO) δ 10.38 (s, 1H), | 505 |
| | fluoro-4-((5-fluoro- | 8.81 (s, 2H), 8.17 (s, 1H), 7.72 (m, 2H), 7.55- | |
| | pyrimidin-2-yl) oxy) | 7.46 (m, 2H), 7.41 (m, 2H), 7.30 (m, 1H), 5.78- | |
| | phenyl)pyrazolo[5,1-f] | 5.66 (m, 1H), 5.46-5.42 (m, 1H). | |
| | [1,2,4]triazin-6- | | |
| | yl)phenyl)-2- | | |
| | fluoroacrylamide | | |
| 336 | 4-(4-amino-6-(4-(2- | ¹H NMR (400 MHz, DMSO) δ 10.36 (s, 1H), | 582 |
| | fluoroacrylamido)phe- | 8.78 (s, 1H), 8.17 (s, 1H), 7.76 (d, J = 7.8 Hz, | |
| | nyl)pyrazolo[5,1- | 1H), 7.68 (d, J = 8.8 Hz, 2H), 7.41 (d, J = 8.8 | |
| | f][1,2,4]triazin-5-yl)-2- | Hz, 2H), 7.21 (d, J = 1.2 Hz, 1H), 7.03 (dd, J = | |
| | methoxy-N-(3- | 8.0, 1.2 Hz, 1H), 5.77 (d, J = 3.6 Hz, 1H), 5.65 | |
| | (trifluoromethyl)bicyclo | (d, J = 3.6 Hz, 1H), 5.46 (d, J = 3.6 Hz, 0.5 H), | |
| | [1.1.1]pentan-1- | 5.42 (d, J = 3.6 Hz, 0.5 H), 3.81 (s, 3H), 2.35 (s, | |
| | yl)benzamide | 6H). | |
| 337 | 4-(4-amino-6-(4-(2- | ¹H NMR (400 MHz, DMSO) δ 10.36 (s, 1H), | 526 |
| | fluoroacrylamido)phe- | 8.30 (d, J = 8.8 Hz, 1H), 8.17 (s, 1H), 7.80 (d, | |
| | nyl)pyrazolo[5,1- | J = 8.0 Hz, 1H), 7.69 (d, J = 8.8 Hz, 2H), 7.43- | |
| | f][1,2,4]triazin-5-yl)-N- | 7.40 (m, 2H), 7.24 (d, J = 1.2 Hz, 1H), 7.04 (dd, | |
| | (1,1-difluoropropan-2- | J = 8.0, 1.4 Hz, 1H), 6.11 (td, J = 56.0, 3.2 Hz, | |
| | yl)-2- | 1H), 5.77 (d, J = 3.6 Hz, 0.5H), 5.65 (d, J = 3.6 | |
| | methoxybenzamide | Hz, 0.5H), 5.46 (d, J = 3.6 Hz, 0.5H), 5.42 (d, | |
| | | J = 3.6 Hz, 0.5H), 4.42 (dd, J = 16.0, 8.0 Hz, 1H), | |
| | | 3.84 (s, 3H), 1.24 (d, J = 7.2 Hz, 3H). | |
| 340 | N-(4-(6-(4- | ¹H NMR (400 MHz, DMSO) δ 10.22 (s, 1H), | 520 |
| | acrylamidophenyl)-4- | 9.50 (s, 1H), 8.20 (d, J = 8.0 Hz, 1H), 8.14 (s, | |
| | aminopyrazolo[5,1- | 1H), 7.61 (d, J = 8.0 Hz, 2H), 7.42 (d, J = 12.0 | |
| | f][1,2,4]triazin-5-yl)-2- | Hz, 2H), 7.11 (m, 1H), 6.97 (m, 1H), 6.42 (m, | |
| | methoxyphenyl)-3,3- | 1H), 6.25 (m, 1H), 5.77-5.74 (m, 1H), 3.38 (m, | |
| | difluorocyclobutane-1- | 1H), 2.78 (m, 4H). | |
| | carboxamide | | |
| 341 | 4-(2-(4-acryl | ¹H NMR (400 MHz, DMSO) δ 10.30 (s, 1H), | 533 |
| | amidophenyl)-4-amino- | 8.44 (s, 1H), 8.36 (s, 1H), 7.71 (d, J = 8.6 Hz, | |
| | 7-cyano-1-methyl-1H- | 2H), 7.64 (d, J = 7.8 Hz, 1H), 7.29 (d, J = 8.5 | |
| | pyrrolo[3,2-c]pyridin-3- | Hz, 2H), 6.94 (s, 1H), 6.87 (d, J = 7.8 Hz, 1H), | |
| | yl)-N-(bicyclo[1.1.1] | 6.41 (d, J = 10.1 Hz, 1H), 6.29 (d, J = 1.8 Hz, | |
| | pentan-1-yl)-2- | 1H), 5.78 (m, 1H), 3.84 (s, 3H), 3.71 (s, 3H), | |
| | methoxybenzamide | 2.44 (s, 1H), 2.05 (s, 6H). | |
| 342 | 4-(2-(4-acryl- | ¹H NMR (400 MHz, DMSO) δ 10.29 (s, 1H), | 575 |
| | amidophenyl)-4-amino- | 8.31 (s, 1H), 8.06 (d, J = 4.4 Hz, 1H), 7.70 (d, | |
| | 7-cyano-1-(2,2,2- | J = 8.5 Hz, 2H), 7.56 (d, J = 7.8 Hz, 1H), 7.31 (d, | |
| | trifluoroethyl)-1H- | J = 8.2 Hz, 2H), 7.00 (s, 1H), 6.86 (s, 1H), 6.40 | |
| | pyrrolo[3,2-c]pyridin-3- | (d, J = 10.1 Hz, 1H), 6.29 (d, J = 1.9 Hz, 1H), | |
| | yl)-N-cyclopropyl-2- | 5.80 (m, 1H), 5.13 (s, 2H), 3.72 (s, 3H), 2.79 | |
| | methoxybenzamide | (m, 1H), 0.69-0.64 (m, 2H), 0.53-0.48 (m, 2H). | |
| 345 | N-(4-(2-(4- | ¹H NMR (400 MHz, DMSO) δ 10.30 (s, 1H), | 495 |
| | acrylamidophenyl)-4- | 10.02 (s, 1H), 8.34 (s, 1H), 7.97 (t, J = 8.4 Hz, | |
| | amino-7-cyano-1- | 1H), 7.71 (d, J = 8.4 Hz, 2H), 7.27 (d, J = 8.4 | |
| | methyl-1H-pyrrolo[3,2- | Hz, 2H), 7.10 (dd, J = 11.6, 1.7 Hz, 1H), 7.00 (d, | |
| | c]pyridin-3-yl)-2- | J = 10.0 Hz, 1H), 6.43 (dd, J = 16.8, 10.0 Hz, | |
| | fluorophenyl)cyclo- | 1H), 6.27 (dd, J = 17.2, 2.0 Hz, 1H), 5.78 (dd, | |
| | propanecarboxamide | J = 10.0, 2.0 Hz, 1H), 3.83 (s, 3H), 2.05-1.99 (m, | |
| | | 1H), 0.79 (d, J = 6.4 Hz, 4H). | |
| 347 | 4-(3-acrylamido-12- | ¹H NMR (400 MHz, DMSO) δ 10.27 (s, 1H), | 583 |
| | amino-9-cyano-6,7- | 8.54 (d, J = 6.8 Hz, 1H), 8.39 (s, 1H), 7.71 (dd, | |
| | dihydro-5H-benzo[c]pyr- | J = 14.4, 4.4 Hz, 2H), 7.42 (dd, J = 8.4, 1.6 Hz, | |
| | ido[3′,4′:4,5]pyr- | 1H), 7.01 (m, 2H), 6.88 (d, J = 8.4 Hz, 1H), 6.43 | |
| | rolo[1,2-a]azepin- | (dd, J = 17.2, 10.0 Hz, 1H), 6.26 (dd, J = 17.2, | |
| | 13-yl)-N-(3,3- | 1.6 Hz, 1H), 5.77 (dd, J = 10.0, 1.6 Hz, 1H), 4.34- | |
| | difluorocyclobutyl)-2- | 4.23 (m, 3H), 3.79 (s, 3H), 2.99-2.89 (m, 2H), | |
| | methoxybenzamide | 2.82-2.67 (m, 4H), 2.30 (m, 2H). | |
| 348 | 4-(4-amino-6-(4-(2- | ¹H NMR (400 MHz, DMSO) δ 10.38 (s, 1H), | 546 |
| | fluoroacrylamido)phe- | 9.08 (t, J = 6.4 Hz, 1H), 8.18 (s, 1H), 7.70 (d, J = | |
| | nyl)pyrazolo[5,1-f][1,2,4] | 8.4 Hz, 2H), 7.55 (d, J = 8.0 Hz, 1H), 7.43-7.35 | |
| | triazin-5-yl)-2-(methylthio)- | (m, 3H), 7.26-7.21 (m, 1H), 5.71 (m, 1H), 5.44 | |
| | N-(2,2,2-trifluoro- | (m, 1H), 4.14-4.00 (m, 2H), 2.27 (s, 3H). | |
| | ethyl)benzamide | | |
| 349 | N-(4-(4-amino-5-(2-(4- | ¹H NMR (400 MHz, DMSO) δ 10.39 (s, 1H), | 488 |
| | methoxyphenyl)thiazol- | 8.19 (s, 1H), 7.95-7.87 (m, 3H), 7.73 (d, J = 8.6 | |
| | 5-yl)pyrazolo[5,1-f] | Hz, 2H), 7.51 (d, J = 8.6 Hz, 2H), 7.07 (d, J = | |
| | [1,2,4]triazin-6- | 8.7 Hz, 2H), 5.77-5.65 (d, J = 3.6 Hz, 1H), 5.43 | |
| | yl)phenyl)-2- | (dd, J = 15.6, 3.6 Hz, 1H), 3.83 (s, 3H). | |
| | fluoroacrylamide | | |
| 350 | N-(4-(4-amino-5-(2-(4- | ¹H NMR (400 MHz, DMSO) δ 10.25 (s, 1H), | 470 |
| | methoxyphenyl)thiazol- | 8.19 (s, 1H), 7.94-7.87 (m, 3H), 7.67 (d, J = 8.6 | |
| | 5-yl)pyrazolo[5,1- | Hz, 2H), 7.50 (d, J = 8.7 Hz, 2H), 7.07 (d, J = | |
| | f][1,2,4]triazin-6- | 8.9 Hz, 2H), 6.41-6.23 (m, 2H), 5.81-5.71 (m, | |
| | yl)phenyl)acrylamide | 1H), 3.83 (s, 3H). | |
| 351 | 4-(2-(4-acryl | ¹H NMR (400 MHz, DMSO) δ 10.27 (s, 1H), | 575 |
| | amidophenyl)-4-amino- | 8.66 (t, J = 6.4 Hz, 1H), 8.40 (s, 1H), 7.74 (dd, | |
| | 7-cyano-1-cyclopropyl- | J = 8.4, 3.6 Hz, 1H), 7.67 (d, J = 8.4 Hz, 2H), 7.34 | |
| | 1H-pyrrolo[3,2-c] | (d, J = 8.4 Hz, 2H), 7.04 (s, 1H), 6.89-6.82 (m, | |
| | pyridin-3-yl)-2- | 1H), 6.48-6.37 (m, 1H), 6.26 (dd, J = 17.2, 2.0 | |
| | methoxy-N-(2,2,2-tri- | Hz, 1H), 5.78 (dd, J = 10.0, 1.6 Hz, 1H), 4.09 | |
| | fluoroethyl)benzamide | (dd, J = 16.0, 9.2 Hz, 2H), 3.87 (m, 1H), 3.77 (s, | |
| | | 3H), 0.96 (d, J = 6.4 Hz, 2H), 0.69 (s, 2H). | |
| 352 | 4-(2-(4-acryl | ¹H NMR (400 MHz, DMSO) δ 10.30 (s, 1H), | 565 |
| | amidophenyl)-4-amino- | 9.02 (t, J = 16.0 Hz, 1H), 8.22 (s, 1H), 7.70 (d, | |
| | 7-cyano-1-methyl-1H- | J = 12.0 Hz, 2H), 7.43 (d, J = 8.0 Hz, 1H), 7.30 (d, | |
| | pyrrolo[3,2-c]pyridin-3- | J = 8.0 Hz, 2H), 7.12 (s, 1H), 7.10-7.06 (m, | |
| | yl)-2-(methylthio)-N- | 1H), 6.43 (m, 1H), 6.27 (m, 1H), 5.78 (m, 1H), | |
| | (2,2,2-trifluoroethyl) | 4.05-3.97 (m, 2H), 3.80 (s, 3H), 2.18 (s, 3H). | |
| | benzamide | | |
| 353 | N-(4-(4-amino-5-(3- | ¹H NMR (400 MHz, DMSO) δ 10.22 (s, 1H), | 526 |
| | methoxy-4-(2-oxo-2- | 8.57-8.54 (m, 1H), 8.15 (s, 1H), 7.62-7.60 (d, J = | |
| | ((2,2,2-trifluoroethyl) | 8.0 Hz, 2H), 7.48-7.43 (d, J = 8.0 Hz, 2H), 7.31- | |
| | amino)ethyl)phe- | 7.29 (d, J = 8.0 Hz, 1H), 7.05 (s, 1H), 6.97- | |
| | nyl)pyrazolo[5,1- | 6.95(d, J = 8.0 Hz, 1H), 6.46-6.39 (m, 1H), 6.28- | |
| | f][1,2,4] | 6.23 (d, J = 20.0 Hz, 1H), 5.78-5.75 (d, J = 12.0 | |
| | triazin-6-yl)phenyl) | Hz, 1H), 3.95-3.91 (m,, 2H), 3.69 (s, 3H), 3.57 | |
| | acrylamide | (s, 2H). | |
| 354 | N-(4-(4-amino-7- | ¹H NMR (400 MHz, DMSO) δ 10.28 (s, 1H), | 563 |
| | cyano-3-(3-methoxy-4- | 8.56 (t, J = 6.4 Hz, 1H), 8.19 (s, 1H), 7.68 (d, J = | |
| | (2-oxo-2-((2,2,2- | 8.4 Hz, 2H), 7.31 (d, J = 8.8 Hz, 2H), 7.14 (d, | |
| | trifluoroethyl)ami- | J = 7.6 Hz, 1H), 6.89-6.73 (m, 2H), 6.43 (m, 1H), | |
| | no)ethyl)phenyl)-1-methyl- | 6.27 (m, 1H), 5.77 (m, 1H), 3.90 (m, 2H), 3.79 | |
| | 1H-pyrrolo[3,2-c] | (s, 3H), 3.61 (s, 3H), 3.46 (s, 2H). | |
| | pyridin-2-yl)phenyl) | | |
| | acrylamide | | |
| 356 | 4-(4-amino-6-(2-fluoro- | ¹H NMR (400 MHz, DMSO) δ 10.57 (s, 1H), | 574 |
| | 4-(2-fluoroacrylamido) | 8.70 (s, 1H), 8.20 (s, 1H), 7.67-7.58 (m, 3H), | |
| | phenyl)pyrazolo[5,1- | 7.46-7.42 (t, J = 8.0 Hz, 1H), 7.08 (s, 1H), 6.91- | |
| | f][1,2,4]triazin-5-yl)-2- | 6.89 (d, J = 8.0 Hz, 1H), 5.81-5.68(dd, J = | |
| | methoxy-N-(1- | 4.0,4.0 Hz, 1H), 5.51-5.46 (dd, J = 4.0,4.0 Hz, | |
| | (trifluoromethyl)cyclo- | 1H), 3.76 (s, 3H), 1.33-1.29 (m, 2H), 1.16 (s, | |
| | propyl)benzamide | 2H). | |
| 357 | 4-(4-amino-6-(2-fluoro- | ¹H NMR (400 MHz, DMSO) δ 10.57 (s, 1H), | 532 |
| | 4-(2-fluoroacryl amido) | 8.47 (s, 1H), 8.20 (s, 1H), 7.68-7.58 (m, 3H), | |
| | phenyl)pyrazolo[5,1- | 7.44 (t, J = 8.4 Hz, 1H), 7.05 (d, J = 1.2 Hz, 1H), | |
| | f][1,2,4]triazin-5-yl)-N- | 6.90 (dd, J = 7.9, 1.4 Hz, 1H), 5.75 (dd, J = 47.6, | |
| | (bicyclo[1.1.1] pentan- | 3.7 Hz, 1H), 5.48 (dd, J = 15.6, 3.8 Hz, 1H), 3.74 | |
| | 1-yl)-2- | (s, 3H), 2.45 (s, 1H), 2.07 (s, 6H). | |
| | methoxybenzamide | | |
| 358 | 4-(4-amino-6-(2-fluoro- | ¹H NMR (400 MHz, DMSO) δ 10.57 (s, 1H), | 562 |
| | 4-(2-fluoroacrylamido) | 8.42 (t, J = 5.6 Hz, 1H), 8.20 (s, 1H), 7.79 (d, J = | |
| | phenyl)pyrazolo[5,1-f] | 8.0 Hz, 1H), 7.68-7.57 (m, 2H), 7.44 (t, J = 8.4 | |
| | [1,2,4]triazin-5-yl)-2- | Hz, 1H), 7.07 (d, J = 1.2 Hz, 1H), 6.91 (dd, J = | |
| | methoxy-N-((1- | 8.0, 1.2 Hz, 1H), 5.74 (dd, J = 47.6, 3.6 Hz, 1H), | |
| | (trifluoromethyl)cyclo- | 5.48 (dd, J = 15.6, 3.6 Hz, 1H), 3.75 (d, J = 8.8 | |
| | propyl)methyl) | Hz, 3H), 3.53 (dd, J = 13.2, 6.8 Hz, 2H), 2.61- | |
| | benzamide | 2.52 (m, 2H). | |
| 359 | 4-(2-(4- | ¹H NMR (400 MHz, DMSO) δ 10.25 (s, 1H), | 533 |
| | acrylamidophenyl)-4- | 8.29 (s, 1H), 8.08 (d, J = 4.4 Hz, 1H), 7.65 (d, | |
| | amino-7-cyano-1- | J = 8.4 Hz, 2H), 7.61 (d, J = 7.6 Hz, 1H), 7.33 (d, | |
| | cyclopropyl-1H- | J = 8.4 Hz, 2H), 6.96 (s, 1H), 6.84 (d, J = 7.6 Hz, | |
| | pyrrolo[3,2-c]pyridin-3- | 1H), 6.43 (dd, J = 16.8, 10.0 Hz, 1H), 6.26 (d, | |
| | yl)-N-cyclopropyl-2- | J = 16.8 Hz, 1H), 5.78 (d, J = 10.0 Hz, 1H), 3.87 | |
| | methoxybenzamide | (m, 1H), 3.71 (s, 3H), 2.81 (d, J = 4.4 Hz, 1H), | |
| | | 0.94 (d, J = 6.4 Hz, 2H), 0.68 (d, J = 5.2 Hz, | |
| | | 4H), 0.53 (s, 2H). | |
| 360 | N-(4-(4-amino-5-(3- | ¹H NMR (400 MHz, DMSO) δ 10.36 (s, 1H), | 544 |
| | methoxy-4-(2-oxo-2- | 8.55 (t, J = 8.0 Hz, 1H), 8.16 (s, 1H), 7.69-7.67 | |
| | ((2,2,2-trifluoroethyl) | (d, J = 8.0 Hz, 2H), 7.46-7.44 (d, J = 8.0 Hz, | |
| | amino)ethyl)phe- | 2H), 7.31-7.29 (d, J = 8.0 Hz, 1H), 7.05 (s, 1H), | |
| | nyl)pyrazolo[5,1-f][1,2,4] | 6.97-6.95 (m, 1H), 5.77-5.65 (m, 1H), 5.46- | |
| | triazin-6-yl)phenyl)-2- | 5.41 (m, 1H), 3.95-3.91 (m, 2H), 3.69 (s, 3H), | |
| | fluoroacrylamide | 3.57 (s, 2H). | |
| 361 | 4-(4-amino-6-(2-fluoro- | ¹H NMR (400 MHz, DMSO) δ 10.57 (s, 1H), | 588 |
| | 4-(2-fluoroacrylamido) | 8.34 (t, J = 6.0 Hz, 1H), 8.20 (s, 1H), 7.73 (d, J = | |
| | phenyl)pyrazolo[5,1- | 8.0 Hz, 1H), 7.68-7.57 (m, 2H), 7.44 (t, J = 8.4 | |
| | f][1,2,4]triazin-5-yl)-2- | Hz, 1H), 7.09 (s, 1H), 6.91 (d, J = 9.2 Hz, 1H), | |
| | methoxy-N-((1-(tri- | 5.74 (dd, J = 47.6, 3.6 Hz, 1H), 5.48 (dd, J = | |
| | fluoromethyl)cyclo- | 15.6, 3.6 Hz, 1H), 3.77 (s, 3H), 3.62 (d, J = 6.0 | |
| | propyl)methyl)benzamide | Hz, 2H), 0.93 (d, J = 4.4 Hz, 4H). | |
| 362 | 4-(4-amino-6-(4-(2- | ¹H NMR (400 MHz, DMSO) δ 10.36 (s, 1H), | 526 |
| | fluoroacrylamido)phe- | 8.51 (s, 1H), 8.17 (s, 1H), 7.81 (d, J = 7.9 Hz, | |
| | nyl)pyrazolo[5,1- | 1H), 7.69 (d, J = 8.7 Hz, 2H), 7.42 (d, J = 8.7 | |
| | f][1,2,4]triazin-5-yl)-N- | Hz, 2H), 7.24 (s, 1H), 7.04 (d, J = 7.8 Hz, 1H), | |
| | (2,2-difluoropropyl)-2- | 5.77 (d, J = 3.7 Hz, 1H), 5.65 (d, J = 3.6 Hz, | |
| | methoxybenzamide | 1H), 5.44 (m,1H), 3.84 (s, 3H), 3.82-3.75 (m, | |
| | | 2H), 1.66 (t, J = 19.0 Hz, 3H). | |
| 364 | N-(4-(4-amino-5-(4- | ¹H NMR (400 MHz, DMSO) δ 10.35 (s, 1H), | 488 |
| | (azetidine-1-carbonyl)- | 8.16 (s, 1H), 7.69-7.66 (d, J = 12.0 Hz, 2H), | |
| | 3-methoxyphenyl) | 7.39-7.36 (m,3H), 7.15-7.14 (d, J = 4.0 Hz, 1H), | |
| | pyrazolo[5,1- | 7.01-6.98 (m, 1H), 5.77-5.64 (m, 1H), 5.46-5.41 | |
| | f][1,2,4]triazin-6- | (m, 1H), 4.04-4.00(t, J = 8.0 Hz, 2H), 3.96- | |
| | yl)phenyl)-2- | 3.93(t, J = 8.0 Hz, 2H), 3.75 (s, 3H), 2.30-2.19 | |
| | fluoroacrylamide | (m, 2H). | |
| 365 | N-(4-(4-amino-5-(3- | ¹H NMR (400 MHz, DMSO) δ 10.38 (s, 1H), | 500 |
| | fluoro-4-((6-methyl- | 8.16 (s, 1H), 7.85-7.74 (m, 1H), 7.72 (d, J = 8.8 | |
| | pyridin-2-yl)oxy) | Hz, 2H), 7.55-7.32 (m, 4H), 7.26 (dd, J = 8.3, | |
| | phenyl)pyrazolo[5,1- | 1.4 Hz, 1H), 7.04 (d, J = 7.3 Hz, 1H), 6.94 (d, | |
| | f][1,2,4]triazin-6-yl) | J = 8.2 Hz, 1H), 5.78-5.65 (m, 1H), 5.51-5.38 | |
| | phenyl)-2- | (m, 1H), 2.35 (s, 3H). | |
| | fluoroacrylamide | | |
| 366 | N-(4-(4-amino-5-(3- | ¹H NMR (400 MHz, DMSO) δ 10.25 (s, 1H), | 482 |
| | fluoro-4-((6-methyl- | 8.16 (s, 1H), 7.78 (t, J = 7.8 Hz, 1H), 7.66 (d, J = | |
| | pyridin-2-yl)oxy) | 8.7 Hz, 2H), 7.57-7.31 (m, 4H), 7.26 (dd, J = | |
| | phenyl)pyrazolo[5,1- | 8.2, 1.5 Hz, 1H), 7.05 (d, J = 7.3 Hz, 1H), 6.94 | |
| | f][1,2,4]triazin-6- | (d, J = 8.2 Hz, 1H), 6.53-6.35 (m, 1H), 6.33- | |
| | yl)phenyl)acrylamide | 6.18 (m, 1H), 5.79-5.74 (m, 1H), 2.36 (s, 3H). | |
| 367 | N-(4-(4-amino-5-(3- | ¹H NMR (400 MHz, DMSO) δ 10.38 (s, 1H), | 501 |
| | fluoro-4-((2- | 8.64 (d, J = 5.8 Hz, 1H), 8.16 (d, J = 9.2 Hz, | |
| | methylpyrimidin-4- | 1H), 7.72 (d, J = 8.8 Hz, 2H), 7.53-7.47 (m, | |
| | yl)oxy)phenyl)pyrazolo | 2H), 7.41 (t, J = 5.6 Hz, 2H), 7.30 (dd, J = 8.2, | |
| | [5,1-f][1,2,4]triazin-6- | 1.3 Hz, 1H), 7.11 (d, J = 5.8 Hz, 1H), 5.78 (d, | |
| | yl)phenyl)-2- | J = 3.7 Hz, 0.5H), 5.66 (d, J = 3.7 Hz, 0.5H), 5.44 | |
| | fluoroacrylamide | (dd, J = 15.6, 3.7 Hz, 1H), 2.51 (s, 3H). | |
| 368 | N-(4-(4-amino-5-(3- | ¹H NMR (400 MHz, DMSO) δ 10.24 (s, 1H), | 483 |
| | fluoro-4-((2- | 8.64 (d, J = 5.8 Hz, 1H), 8.16 (s, 1H), 7.66 (d, | |
| | methylpyrimidin-4- | J = 8.7 Hz, 2H), 7.54-7.46 (m, 2H), 7.40 (d, J = | |
| | yl)oxy)phenyl)pyrazolo | 8.7 Hz, 2H), 7.33-7.25 (m, 1H), 7.11 (d, J = 5.7 | |
| | [5,1-f][1,2,4]triazin-6- | Hz, 1H), 6.41 (d, J = 10.1 Hz, 1H), 6.28 (d, J = | |
| | yl)phenyl)acrylamide | 1.9 Hz, 1H), 5.78-5.74 (m, 1H), 2.51 (s, 3H). | |
| 369 | N-(4-(4-amino-5-(1- | ¹H NMR (400 MHz, DMSO) δ 10.20 (s, 1H), | 506 |
| | oxo-2-(2,2,2- | 8.30 (d, J = 8.0 Hz, 1H), 8.17 (s, 1H), 7.73 (s, | |
| | trifluoroethyl)-1,2- | 1H), 7.60-7.58 (d, J = 8.0 Hz, 2H), 7.52-7.50 (d, | |
| | dihydroisoquinolin-6- | J = 8.0 Hz, 2H), 7.35-7.32 (d, J = 12.0 Hz, 2H), | |
| | yl)pyrazolo[5,1- | 6.74-6.72(d, J = 8.0 Hz, 1H), 6.44-6.38 (m, 1H), | |
| | f][1,2,4]triazin-6- | 6.26-6.21 (m, 1H), 5.76-5.73 (m, 1H), 4.97- | |
| | yl)phenyl)acrylamide | 4.92(m, 2H). | |
| 371 | N-(4-(4-amino-5-(1- | ¹H NMR (400 MHz, DMSO) δ 10.21 (s, 1H), | 494 |
| | oxo-2-(2,2,2- | 8.16 (s, 1H), 7.83 (d, J = 7.8 Hz, 1H), 7.70 (s, | |
| | trifluoroethyl)isoindolin- | 1H), 7.60 (d, J = 8.8 Hz, 2H), 7.49 (d, J = 7.6 | |
| | 5-yl)pyrazolo[5,1- | Hz, 1H), 7.35 (d, J = 8.7 Hz, 2H), 6.42 (dd, J = | |
| | f][1,2,4]triazin-6- | 16.9, 10.1 Hz, 1H), 6.24 (dd, J = 17.0, 2.0 Hz, | |
| | yl)phenyl)acrylamide | 1H), 5.75 (dd, J = 10.1, 1.9 Hz, 1H), 4.65 (s, | |
| | | 2H), 4.43 (d, J = 9.4 Hz, 2H). | |
| 372 | 4-(4-amino-6-(4-(2- | ¹H NMR (400 MHz, DMSO) δ 10.36 (s, 1H), | 523 |
| | fluoroacrylamido)phe- | 8.50 (s, 1H), 8.17 (s, 1H), 7.84 (d, J = 7.8 Hz, | |
| | nyl)pyrazolo[5,1- | 1H), 7.68 (d, J = 8.7 Hz, 2H), 7.42 (d, J = 8.7 | |
| | f][1,2,4]triazin-5-yl)-N- | Hz,2H), 7.23 (d, J = 1.4 Hz, 1H), 7.03 (dd, J = | |
| | ((1-fluorocyclo- | 7.9, 1.4 Hz, 1H), 5.77 (d, J = 3.7 Hz, 0.5H), 5.65 | |
| | propyl)methyl)- | (d, J = 3.6 Hz, 0.5H), 5.47-5.40 (m, 1H), 3.76 | |
| | 2-(methoxy- | (dd, J = 20.7, 5.9 Hz, 2H), 1.01 (d, J = 19.2 Hz, | |
| | d3)benzamide | 2H), 0.83 (d, J = 6.6 Hz, 2H). | |
| 373 | N-(4-(4-amino-5-(3- | ¹H NMR (400 MHz, DMSO) δ 10.38 (s, 1H), | 501 |
| | fluoro-4-((6- | 8.46 (s, 1H), 8.35 (s, 1H), 8.17 (s, 1H), 7.72 (d, | |
| | methylpyrazin-2- | J = 8.7 Hz, 2H), 7.57-7.46 (m, 2H), 7.41 (d, J = | |
| | yl)oxy)phenyl)pyrazolo | 8.7 Hz, 2H), 7.29 (d, J = 8.3 Hz, 1H), 5.72 (dd, | |
| | [5,1-f][1,2,4]triazin-6- | J = 47.6, 3.6 Hz, 1H), 5.44 (dd, J = 15.6, 3.6 Hz, | |
| | yl)phenyl)-2- | 1H), 2.41 (s, 3H). | |
| | fluoroacrylamide | | |
| 374 | N-(4-(4-amino-5-(3- | ¹H NMR (400 MHz, DMSO) δ 10.24 (s, 1H), | 483 |
| | fluoro-4-((6- | 8.46 (s, 1H), 8.36 (s, 1H), 8.16 (s, 1H), 7.66 (d, | |
| | methylpyrazin-2- | J = 8.7 Hz, 2H), 7.58-7.45 (m, 2H), 7.39 (d, J = | |
| | yl)oxy)phenyl)pyrazolo | 8.7 Hz, 2H), 7.28 (dd, J = 8.3, 1.4 Hz, 1H), 6.43 | |
| | [5,1-f][1,2,4]triazin-6- | (dd, J = 17.0, 10.0 Hz, 1H), 6.26 (dd, J = 17.0, | |
| | yl)phenyl)acrylamide | 2.0 Hz, 1H), 5.77 (dd, J = 10.1, 2.0 Hz, 1H), 2.41 | |
| | | (s, 3H). | |
| 375 | N-(4-(4-amino-5-(3- | ¹H NMR (400 MHz, DMSO) δ 10.38 (s, 1H), | 501 |
| | fluoro-4-((5-methyl- | 8.54 (s, 2H), 8.17 (s, 1H), 7.73-7.71 (d, J = | |
| | pyrimidin-2-yl)oxy) | 8.0 Hz, 2H), 7.51-7.41 (m, 4H), 7.30-7.27 (m, | |
| | phenyl)pyrazolo[5,1- | 1H), 5.78-5.66 (m, 1H), 5.47-5.42 (m, 1H), | |
| | f][1,2,4]triazin-6-yl) | 2.25 (s, 3H). | |
| | phenyl)-2- | | |
| | fluoroacrylamide | | |
| 376 | N-(4-(4-amino-5-(3- | ¹H NMR (400 MHz, DMSO) δ 10.25 (s, 1H), | 483 |
| | fluoro-4-((5- | 8.55 (s, 2H), 8.17 (s, 1H), 7.67-7.65(d, J = 8.0 | |
| | methylpyrimidin-2- | Hz, 2H), 7.47-7.39 (m, 4H), 7.30-7.27 (dd, J = | |
| | yl)oxy)phenyl)pyrazolo | 4.0, 4.0 Hz, 1H), 6.47-6.40 (m, 1H), 6.29-6.24 | |
| | [5,1-f][1,2,4]triazin-6- | (m, 1H), 5.78-5.75 (m, 1H), 2.25 (s, 3H). | |
| | yl)phenyl)acrylamide | | |
| 377 | N-(4-(4-amino-5-(3- | ¹H NMR (400 MHz, DMSO) δ 10.39 (s, 1H), | 487 |
| | fluoro-4-(pyrimidin-2- | 8.72 (d, J = 4.8 Hz, 2H), 8.17 (s, 1H), 7.72 (d, | |
| | yloxy)phenyl)pyrazolo[5,1- | J = 8.8 Hz, 2H), 7.55-7.45 (m, 2H), 7.42 (d, J = | |
| | f][1,2,4]triazin-6- | 8.8 Hz, 2H), 7.36 (m, 1H), 7.30 (m, 1H), 5.72 | |
| | yl)phenyl)-2- | (dd, J = 47.7, 3.7 Hz, 1H), 5.44 (dd, J = 15.6, 3.7 | |
| | fluoroacrylamide | Hz, 1H). | |
| 378 | N-(4-(4-amino-3-(3- | ¹H NMR (400 MHz, DMSO) δ 8.48 (d, J = 5.0 | 497 |
| | fluoro-4-((4-methyl- | Hz, 1H), 8.26 (s, 1H), 7.59 (d, J = 11.1 Hz, 1H), | |
| | pyrimidin-2-yl)oxy) | 7.34 (d, J = 8.3 Hz, 1H), 7.20 (d, J = 5.0 Hz, | |
| | phenyl)-2H-pyrazolo | 2H), 7.05 (d, J = 8.6 Hz, 2H), 6.55 (d, J = 8.7 | |
| | [3,4-d]pyrimidin-2-yl) | Hz, 2H), 5.95 (s, 1H), 5.52 (s, 2H), 5.48 (s, 1H), | |
| | phenyl)methacrylamide | 2.42 (s, 3H), 1.80 (s, 3H). | |
| 379 | N-(4-(4-amino-5-(3- | ¹H NMR (400 MHz, DMSO) δ 10.38 (s, 1H), | 517 |
| | fluoro-4-((4- | 8.38 (d, J = 5.7 Hz, 1H), 8.17 (s, 1H), 7.70 (d, | |
| | methoxypyrimidin-2- | J = 8.8 Hz, 2H), 7.48 (ddd, J = 25.5, 19.0, 8.5 Hz, | |
| | yl)oxy)phenyl)pyrazolo | 4H), 7.29 (d, J = 6.8 Hz, 1H), 6.76 (d, J = 5.7 | |
| | [5,1-f][1,2,4]triazin-6- | Hz, 1H), 5.78 (d, J = 3.7 Hz, 1H), 5.66 (d, J = | |
| | yl)phenyl)-2- | 3.6 Hz, 1H), 5.45 (dd, J = 15.6, 3.7 Hz, 1H), 3.86 | |
| | fluoroacrylamide | (s, 3H). | |
| 380 | N-(4-(4-amino-5-(4-((4- | ¹H NMR (400 MHz, DMSO) δ 10.38 (s, 1H), | 537 |
| | (difluoromethyl)pyr- | 8.94 (d, J = 4.9 Hz, 1H), 8.17 (s, 1H), 7.72 (d, | |
| | imidin-2-yl)oxy)-3- | J = 8.8 Hz, 2H), 7.63 (d, J = 5.0 Hz, 1H), 7.59- | |
| | fluorophenyl)pyrazolo[5,1- | 7.48 (m, 2H), 7.42 (d, J = 8.7 Hz, 2H), 7.32 (d, | |
| | f][1,2,4]triazin-6- | J = 8.3 Hz, 1H), 6.97 (t, J = 54.0 Hz, 1H), 5.78 (d, | |
| | yl)phenyl)-2- | J = 3.6 Hz, 1H), 5.44 (dd, J = 15.6, 3.7 Hz, 1H). | |
| | fluoroacrylamide | | |
| 381 | N-(4-(4-amino-5-(3- | ¹H NMR (400 MHz, DMSO) δ 10.38 (s, 1H), | 501 |
| | fluoro-4-((6-methyl- | 8.70 (s, 1H), 8.17 (s, 1H), 7.72 (d, J = 8.8 Hz, | |
| | pyrimidin-4-yl)oxy) | 2H), 7.50 (s, 2H), 7.41 (d, J = 8.8 Hz, 2H), 7.30 | |
| | phenyl)pyrazolo[5,1- | (d, J = 7.8 Hz, 1H), 7.20 (s, 1H), 5.78 (s, 0.5H), | |
| | f][1,2,4]triazin-6-yl) | 5.67 (s, 0.5H), 5.44 (d, J = 15.5 Hz, 1H), 3.30 (s, | |
| | phenyl)-2- | 3H). | |
| | fluoroacrylamide | | |
| 382 | N-(4-(4-amino-5-(3- | ¹H NMR (400 MHz, DMSO) δ 10.25 (s, 1H), | 483 |
| | fluoro-4-((6- | 8.70 (s, 1H), 8.17 (s, 1H), 7.66 (d, J = 8.7 Hz, | |
| | methylpyrimidin-4- | 2H), 7.49 (d, J = 8.5 Hz, 2H), 7.39 (d, J = 8.7 | |
| | yl)oxy)phenyl)pyrazolo | Hz, 2H), 7.29 (d, J = 9.6 Hz, 1H), 7.20 (s, 1H), | |
| | [5,1-f][1,2,4]triazin-6- | 6.41 (d, J = 10.1 Hz, 1H), 6.28 (d, J = 1.8 Hz, | |
| | yl)phenyl)acrylamide | 1H), 5.77 (d, J = 10.0 Hz, 1H), 3.45 (s, 3H). | |
| 383 | 4-(4-amino-6-(4-(2- | ¹H NMR (400 MHz, DMSO) δ 10.36 (s, 1H), | 490 |
| | fluoroacrylamido)phe- | 8.93 (t, J = 6.0 Hz, 1H), 8.17 (s, 1H), 8.01 (d, J = | |
| | nyl)pyrazolo[5,1- | 8.4 Hz, 2H), 7.67 (d, J = 8.8 Hz, 2H), 7.51 (d, | |
| | f][1,2,4]triazin-5-yl)-N- | J = 8.4 Hz, 2H), 7.36 (d, J = 8.8 Hz, 2H), 5.71 (dd, | |
| | ((1-fluorocyclopropyl) | J = 47.6, 3.6 Hz, 1H), 5.43 (dd, J = 15.6, 3.6 Hz, | |
| | methyl)benzamide | 1H), 3.76 (d, J = 6.0 Hz, 1H), 3.71 (d, J = 6.0 | |
| | | Hz, 1H), 1.01 (dt, J = 13.6, 6.4 Hz, 2H), 0.84 (dt, | |
| | | J = 14.8, 7.2 Hz, 2H). | |
| 384 | N-(4-(4-amino-5-(3- | ¹H NMR (400 MHz, DMSO) δ 10.38 (s, 1H), | 517 |
| | fluoro-4-((4- | 8.48 (d, J = 8.0 Hz, 1H), 8.18 (s, 1H), 7.79-7.72 | |
| | methylpyrimidin-2-yl) | (m, 3H), 7.47-7.41 (m, 3H), 7.33-7.30 (dd, J = | |
| | thio)phenyl)pyrazolo[5, | 4.0 Hz, 1H), 7.19-7.18 (d, J = 4.0 Hz, 1H), 5.78- | |
| | 1-f][1,2,4]triazin-6- | 5.65(dd, J = 4.0 Hz, 1H), 5.47-5.42 (d, J = 4.0 | |
| | yl)phenyl)-2- | Hz, 1H), 2.39 (s, 3H). | |
| | fluoroacrylamide | | |
| 385 | N-(4-(4-amino-5-(3- | ¹H NMR (400 MHz, DMSO) δ 10.24 (s, 1H), | 499 |
| | fluoro-4-((4-methyl- | 8.49-8.48 (d, J = 4.0 Hz, 1H), 8.18 (s, 1H), 7.79- | |
| | pyrimidin-2-yl)thio) | 7.75 (t, J = 8.0 Hz, 1H), 7.67-7.65 (d, J = 8.0 Hz, | |
| | phenyl)pyrazolo[5,1- | 2H), 7.47-7.39 (m, 3H), 7.33-7.31 (m, 1H), | |
| | f][1,2,4]triazin-6-yl) | 7.19-7.18 (d, J = 4.0 Hz, 1H), 6.47-6.40 (m, 1H), | |
| | phenyl)-2- | 6.29-6.24 (m, 1H), 5.78-5.75(m, 1H), 2.39 (s, | |
| | fluoroacrylamide | 3H). | |
| 386 | N-(4-(4-amino-5-(4-((4- | ¹H NMR (400 MHz, DMSO) δ 10.39 (s, 1H), | 527 |
| | cyclopropylpyrimidin- | 8.46 (d, J = 5.1 Hz, 1H), 8.17 (s, 1H), 7.75 (d, | |
| | 2-yl)oxy)-3-fluoro- | J = 8.8 Hz, 2H), 7.46 (m, 4H), 7.31-7.24 (m, 2H), | |
| | phenyl)pyrazolo[5,1-f] | 5.77 (d, J = 3.7 Hz, 0.5H), 5.65 (d, J = 3.7 Hz, | |
| | [1,2,4]triazin-6-yl) | 0.5H), 5.44 (dd, J = 15.6, 3.7 Hz, 1H), 2.13 (m, | |
| | phenyl)-2- | 1H), 1.10 (m, 2H), 0.93-0.83 (m, 2H). | |
| | fluoroacrylamide | | |
| 387 | N-(4-(4-amino-5-(4-((4- | ¹H NMR (400 MHz, DMSO) δ 10.24 (s, 1H), | 509 |
| | cyclopropylpyrimidin- | 8.47 (d, J = 5.1 Hz, 1H), 8.16 (s, 1H), 7.68 (d, | |
| | 2-yl)oxy)-3- | J = 8.8 Hz, 2H), 7.45 (m, 4H), 7.27 (d, J = 5.2 Hz, | |
| | fluorophenyl)pyrazolo[5,1- | 2H), 6.47-6.40 (m, 1H), 6.25 (dd, J = 17.0, 2.0 | |
| | f][1,2,4]triazin-6- | Hz, 1H), 5.76 (dd, J = 10.1, 2.0 Hz, 1H), 2.13 | |
| | yl)phenyl)acrylamide | (m, 1H), 1.11 (m, 2H), 0.90-0.86 (m, 2H). | |
| 388 | N-(4-(4-amino-5-(3- | ¹H NMR (400 MHz, DMSO) δ 10.39 (s, 1H), | 526 |
| | fluoro-4-(imidazo[1,2- | 8.18 (s, 1H), 8.13 (s, 1H), 7.75 (m, 4H), 7.69 (d, | |
| | c]pyrimidin-5-yloxy) | J = 6.5 Hz, 1H), 7.58 (m, 1H), 7.42 (m, 3H), | |
| | phenyl)pyrazolo[5,1- | 7.36 (d, J = 8.1 Hz, 1H), 5.78 (d, J = 3.7 Hz, | |
| | f][1,2,4]triazin-6-yl) | 0.5H), 5.66 (d, J = 3.7 Hz, 0.5H), 5.45 (m, 1H). | |
| | phenyl)-2- | | |
| | fluoroacrylamide | | |
| 389 | N-(4-(4-amino-5-(3- | ¹H NMR (400 MHz, DMSO) δ 10.09 (s, 1H), | 515 |
| | fluoro-4-((4-methyl- | 8.49 (d, J = 5.0 Hz, 1H), 8.20 (s, 1H), 7.68 (dd, | |
| | pyrimidin-2-yl)oxy) | J = 12.7, 1.9 Hz, 1H), 7.54 (dd, J = 8.5, 1.9 Hz, | |
| | phenyl)pyrazolo[5,1- | 1H), 7.41 (t, J = 8.5 Hz, 2H), 7.32 (dd, J = 11.2, | |
| | f][1,2,4]triazin-6-yl)-3- | 2.0 Hz, 1H), 7.23-7.13 (m, 2H), 5.82 (s, 1H), | |
| | fluorophenyl)methacrylamide | 5.57 (s, 1H), 2.43 (s, 3H), 1.95 (s, 3H). | |
| 390 | 4-(4-amino-6-(4-(2- | ¹H NMR (400 MHz, DMSO) δ 10.38 (s, 1H), | 524 |
| | fluoroacrylamido)phe- | 10.17 (s, 1H), 8.18 (s, 1H), 7.74 (m, 5H), 7.46 | |
| | nyl)pyrazolo[5,1-f][1,2,4] | (d, J = 8.7 Hz, 2H), 7.36 (s, 2H), 7.28 (s, 1H), | |
| | triazin-5-yl)-2-methoxy- | 7.13-7.06 (m, 2H), 5.77 (d, J = 3.7 Hz, 0.5 H), | |
| | N-phenylbenzamide | 5.65 (d, J = 3.7 Hz, 0.5 H), 5.44 (m, 1H), 3.86 (s, | |
| | | 3H). | |
| 391 | 4-(4-amino-6-(4-(2- | ¹H NMR (400 MHz, DMSO) δ 10.51 (s, 1H), | 525 |
| | fluoroacrylamido)phe- | 10.37 (s, 1H), 8.37 (d, J = 3.6 Hz, 1H), 8.28 (d, | |
| | nyl)pyrazolo[5,1- | J = 8.4 Hz, 1H), 8.18 (s, 1H), 7.93 (d, J = 7.9 Hz, | |
| | f][1,2,4]triazin-5-yl)-2- | 1H), 7.88 (t, J = 7.8 Hz, 1H), 7.70 (d, J = 8.8 Hz, | |
| | methoxy-N-(pyridin-2- | 2H), 7.45 (d, J = 8.8 Hz, 2H), 7.35 (s, 1H), 7.19 | |
| | yl)benzamide | (dd, J = 7.3, 4.9 Hz, 1H), 7.08 (d, J = 7.8 Hz, | |
| | | 1H), 5.71 (dd, J = 47.7, 3.7 Hz, 1H), 5.43 (dd, | |
| | | J = 15.6, 3.7 Hz, 1H), 3.95 (s, 3H). | |
| 392 | N-(4-(4-amino-5-(3- | ¹H NMR (400 MHz, DMSO) δ 10.38 (s, 1H), | 514 |
| | fluoro-4-(methyl(4- | 8.28-8.26 (d, J = 8.0 Hz, 1H), 8.18 (s, 1H), 7.73- | |
| | methylpyrimidin-2-yl)ami- | 7.71 (d, J = 8.0 Hz, 2H), 7.56-7.52 (t, J = 8.0 Hz, | |
| | no)phenyl)pyrazolo[5,1- | 1H), 7.45-7.43 (d, J = 8.0 Hz, 2H), 7.37-7.34 (m, | |
| | f][1,2,4]triazin- | 1H), 7.29-7.26 (m, 1H), 6.71-6.69 (d, J = 8.0 Hz, | |
| | 6-yl)phenyl)-2- | 1H), 5.78-5.65 (m, 1H), 5.47-5.42 (m, 1H), | |
| | fluoroacrylamide | 3.48 (s, 3H), 2.31 (s, 3H). | |
| 393 | N-(4-(4-amino-5-(3- | ¹H NMR (400 MHz, DMSO) δ 10.39 (s, 1H), | 555 |
| | fluoro-4-((4- | 9.10 (d, J = 5.1 Hz, 1H), 8.17 (s, 1H), 7.89 (d, | |
| | (trifluoromethyl)pyr- | J = 4.9 Hz, 1H), 7.71 (d, J = 8.7 Hz, 2H), 7.64- | |
| | imidin-2-yl)oxy)phenyl) | 7.49 (m, 2H), 7.42 (d, J = 8.8 Hz, 2H), 7.32 (d, | |
| | pyrazolo[5,1-f][1,2,4] | J = 9.5 Hz, 1H), 5.72 (dd, J = 47.8, 3.7 Hz, 1H), | |
| | triazin-6-yl)phenyl)-2- | 5.44 (dd, J = 15.6, 3.6 Hz, 1H). | |
| | fluoroacrylamide | | |
| 394 | N-(4-(4-amino-5-(3- | ¹H NMR (400 MHz, DMSO) δ 10.24 (s, 1H), | 537 |
| | fluoro-4-((4-(trifluoro- | 9.10 (d, J = 4.8 Hz, 1H), 8.17 (s, 1H), 7.89 (d, | |
| | methyl)pyrimidin-2- | J = 5.2 Hz, 1H), 7.66 (s, 1H), 7.64 (s, 1H), 7.59 (t, | |
| | yl)oxy)phenyl)pyrazolo | J = 8.4 Hz, 1H), 7.53 (dd, J = 11.2, 2.0 Hz, 1H), | |
| | [5,1-f][1,2,4]triazin-6- | 7.41 (s, 1H), 7.39 (s, 1H), 7.32 (dd, J = 8.4, 1.2 | |
| | yl)phenyl)acrylamide | Hz, 1H), 6.44 (dd, J = 17.2, 10.0 Hz, 1H), 6.26 | |
| | | (dd, J = 17.2, 2.0 Hz, 1H), 5.77 (dd, J = 10.0, 2.0 | |
| | | Hz, 1H). | |
| 395 | 4-(4-amino-6-(4-(but-2- | ¹H NMR (400 MHz, DMSO) δ 10.70 (s, 1H), | 550 |
| | ynamido)phenyl)pyrazolo[5,1- | 8.77 (s, 1H), 8.16 (s, 1H), 7.70 (d, J = 7.8 Hz, | |
| | f][1,2,4]triazin- | 1H), 7.54 (d, J = 8.7 Hz, 2H), 7.38 (d, J = 8.7 | |
| | 5-yl)-2-methoxy-N-(1- | Hz, 2H), 7.21 (d, J = 1.0 Hz, 1H), 7.00 (dd, J = | |
| | (trifluoromethyl)cyclo- | 7.8, 1.2 Hz, 1H), 3.81 (s, 3H), 2.04 (s, 3H), 1.40- | |
| | propyl)benzamide | 1.29 (m, 2H), 1.23-1.12 (m, 2H). | |
| 396 | 4-(4-amino-6-(4- | ¹H NMR (400 MHz, DMSO) δ 10.91 (s, 1H), | 536 |
| | propiolamidophenyl)pyr- | 8.77 (s, 1H), 8.16 (s, 1H), 7.70 (d, J = 7.8 Hz, | |
| | azolo[5,1- | 1H), 7.55 (d, J = 8.7 Hz, 2H), 7.40 (d, J = 8.7 | |
| | f][1,2,4]triazin-5-yl)-2- | Hz, 2H), 7.21 (d, J = 1.0 Hz, 1H), 7.01 (dd, J = | |
| | methoxy-N-(1- | 7.8, 1.2 Hz, 1H), 4.45 (s, 1H), 3.81 (s, 3H), 1.36- | |
| | (trifluoromethyl)cyclo- | 1.29 (m, 2H), 1.22-1.16 (m, 2H). | |
| | propyl)benzamide | | |
| 397 | 4-(4-amino-6-(4-(2- | ¹H NMR (400 MHz, DMSO) δ 10.37 (s, 1H), | 504 |
| | fluoroacrylamido)phe- | 8.16 (s, 1H), 7.89 (s, 1H), 7.83 (d, J = 8.0 Hz, | |
| | nyl)pyrazolo[5,1- | 1H), 7.68 (d, J = 8.8 Hz, 2H), 7.42 (d, J = 8.8 | |
| | f][1,2,4]triazin-5-yl)-N- | Hz, 2H), 7.21 (d, J = 1.2 Hz, 1H), 7.02 (dd, J = | |
| | (tert-butyl)-2- | 8.0, 1.6 Hz, 1H), 5.77 (d, J = 3.6 Hz, 0.5H), 5.65 | |
| | methoxybenzamide | (d, J = 3.6 Hz, 0.5H), 5.46 (d, J = 3.6 Hz, 0.5H), | |
| | | 5.42 (d, J = 3.6 Hz, 0.5H), 3.84 (s, 3H), 1.40 (s, | |
| | | 9H). | |
| 399 | N-(4-(4-amino-5-(4-((4- | ¹H NMR (400 MHz, DMSO) δ 10.39 (s, 1H), | 515 |
| | ethylpyrimidin-2- | 8.56 (d, J = 4.8 Hz, 1H), 8.17 (s, 1H), 7.72 (d, | |
| | yl)oxy)-3-fluoro- | J = 8.8 Hz, 2H), 7.53-7.41 (m, 4H), 7.29 (d, J = | |
| | phenyl)pyrazolo[5,1- | 8.0 Hz, 1H), 7.23 (d, J = 5.2 Hz, 1H), 5.72 (m, | |
| | f][1,2,4]triazin-6-yl) | 1H), 5.44 (m, 1H), 2.73 (m, 2H), 1.19 (m, 3H). | |
| | phenyl)-2- | | |
| | fluoroacrylamide | | |
| 400 | N-(4-(4-amino-5-(4-((4- | ¹H NMR (400 MHz, DMSO) δ 10.24 (s, 1H), | 497 |
| | ethylpyrimidin-2- | 8.56 (d, J = 4.8 Hz, 1H), 8.16 (s, 1H), 7.65 (d, | |
| | yl)oxy)-3- | J = 8.8 Hz, 2H), 7.53-7.45 (m, 2H), 7.41 (d, J = | |
| | fluorophenyl)pyrazolo[5,1- | 8.8 Hz, 2H), 7.29 (d, J = 8.4 Hz, 1H), 7.23 (d, | |
| | f][1,2,4]triazin-6- | J = 4.8 Hz, 1H), 6.43 (m, 1H), 6.26 (m, 1H), 5.77 | |
| | yl)phenyl)acrylamide | (m, 1H), 2.73 (m, 2H), 1.19 (m, 5H). | |
| 401 | N-(4-(4-amino-5-(3- | ¹H NMR (400 MHz, DMSO) δ 10.37 (s, 1H), | 489 |
| | fluoro-4-((1-methyl- | 8.15 (s, 1H), 7.68 (dd, J = 10.8, 5.5 Hz, 3H), | |
| | 1H-pyrazol-3- | 7.45-7.34 (m, 3H), 7.28 (t, J = 8.5 Hz, 1H), | |
| | yl)oxy)phenyl)pyrazolo | 7.16 (d, J = 8.3 Hz, 1H), 5.94 (d, J = 2.3 Hz, | |
| | [5,1-f][1,2,4]triazin-6- | 1H), 5.71 (dd, J = 47.7, 3.7 Hz, 1H), 5.44 (dd, | |
| | yl)phenyl)-2- | J = 15.6, 3.6 Hz, 1H), 3.76 (s, 3H). | |
| | fluoroacrylamide | | |
| 402 | N-(4-(4-amino-5-(3- | ¹H NMR (400 MHz, DMSO) δ 10.24 (s, 1H), | 471 |
| | fluoro-4-((1-methyl- | 8.14 (s, 1H), 7.65 (dd, J = 13.3, 5.5 Hz, 3H), | |
| | 1H-pyrazol-3- | 7.44-7.33 (m, 3H), 7.28 (t, J = 8.6 Hz, 1H), | |
| | yl)oxy)phenyl)pyrazolo | 7.16 (d, J = 8.3 Hz, 1H), 6.43 (dd, J = 17.0, 10.1 | |
| | [5,1-f][1,2,4]triazin-6- | Hz, 1H), 6.26 (dd, J = 17.0, 2.0 Hz, 1H), 5.94 (d, | |
| | yl)phenyl)acrylamide | J = 2.3 Hz, 1H), 5.76 (dd, J = 10.1, 2.0 Hz, 1H), | |
| | | 3.76 (s, 3H). | |
| 403 | N-(4-(4-amino-5-(3- | ¹H NMR (400 MHz, DMSO) δ 10.37 (s, 1H), | 500 |
| | fluoro-4-((4- | 9.12 (s, 1H), 8.36-8.35 (d, J = 4.0 Hz, 1H), 8.20- | |
| | methylpyrimidin-2- | 8.16(m, 2H), 7.71-7.69 (d, J = 8.0 Hz, 2H), 7.44- | |
| | yl)amino)phenyl)pyrazolo[5,1- | 7.42 (d, J = 8.0 Hz, 2H), 7.31-7.28 (m, 1H), | |
| | f][1,2,4]triazin- | 7.21-7.19 (m, 1H), 6.82-6.80 (d, J = 8.0 Hz, 1H), | |
| | 6-yl)phenyl)-2- | 5.77-5.64 (m, 1H), 5.46-5.41 (m, 1H), 2.37 (s, | |
| | fluoroacrylamide | 3H). | |
| 404 | N-(4-(4-amino-5-(3- | ¹H NMR (400 MHz, MeOD) δ 8.29 (m, 2H), | 482 |
| | fluoro-4-((4- | 8.08 (s, 1H), 7.62-7.60 (d, J = 8.0 Hz, 2H), 7.51- | |
| | methylpyrimidin-2- | 7.48 (d, J = 12.0 Hz, 2H), 7.26-7.23 (m, 2H), | |
| | yl)amino)phenyl)pyrazolo[5,1- | 6.81-6.80 (d, J = 4.0 Hz, 1H), 6.45-6.33 (m, | |
| | f][1,2,4]triazin- | 2H), 5.78-5.75 (m, 1H), 2.44 (s, 3H). | |
| | 6-yl)phenyl)acrylamide | | |
| 405 | N-(4-(4-amino-5-(3- | ¹H NMR (400 MHz, DMSO) δ 10.38 (s, 1H), | 500 |
| | fluoro-4-((4-methyl- | 8.17 (s, 1H), 8.05-8.03 (d, J = 8.0 Hz, 1H), 7.73- | |
| | pyridin-2-yl)oxy) | 7.71 (d, J = 8.0 Hz, 2H), 7.46-7.39 (m, 4H), | |
| | phenyl)pyrazolo[5,1-f] | 7.27-7.25 (m, 1H), 7.03-7.00 (m, 2H), 5.79- | |
| | [1,2,4]triazin-6-yl) | 5.66 (m, 1H), 5.47-5.42 (m, 1H), 2.37 (s, 3H). | |
| | phenyl)-2- | | |
| | fluoroacrylamide | | |
| 406 | N-(4-(4-amino-5-(3- | ¹H NMR (400 MHz, DMSO) δ 10.25 (s, 1H), | 482 |
| | fluoro-4-((4- | 8.16 (s, 1H), 8.05-8.03 (d, J = 8.0 Hz, 1H), 7.67- | |
| | methylpyridin-2- | 7.64(d, J = 12.0 Hz, 2H), 7.46-7.39(m, 4H), | |
| | yl)oxy)phenyl)pyrazolo | 7.27-7.25 (m, 1H), 7.03-7.00 (m, 2H), 6.47- | |
| | [5,1-f][1,2,4]triazin-6- | 6.40 (m, 1H), 6.29-6.24 (m, 1H), 5.78-5.75 (m, | |
| | yl)phenyl)acrylamide | 1H), 2.37 (s, 3H). | |
| 407 | N-(4-(4-amino-5-(4-(4- | ¹H NMR (400 MHz, DMSO) δ 10.24 (s, 1H), | 509 |
| | fluorobenzoyl)-3- | 8.17 (s, 1H), 7.93 (m, 2H), 7.66 (d, J = 8.8 Hz, | |
| | methoxyphenyl)pyrazolo[5,1- | 2H), 7.43-7.35 (m, 5H), 7.24 (s, 1H), 7.08- | |
| | f][1,2,4]triazin-6- | 7.03 (m, 1H), 6.44 (m, 1H), 6.26 (dd, J = 17.0, | |
| | yl)phenyl)acrylamide | 2.0 Hz, 1H), 5.77 (m, 1H), 3.62 (s, 3H). | |
| 408 | N-(4-(4-amino-5-(3- | ¹H NMR (400 MHz, DMSO) δ 9.88 (s, 1H), 8.16 | 496 |
| | fluoro-4-((6-methyl- | (s, 1H), 7.78 (dd, J = 8.0, 7.6 Hz, 1H), 7.73- | |
| | pyridin-2-yl)oxy) | 7.63 (m, 2H), 7.46-7.36 (m, 4H), 7.2 (dd, J = | |
| | phenyl)pyrazolo[5,1- | 8.4, 1.3 Hz, 1H), 7.04 (d, J = 7.2 Hz, 1H), 6.94 | |
| | f][1,2,4]triazin-6-yl) | (d, J = 8.4 Hz, 1H), 5.78 (s, 1H), 5.52 (s, 1H), | |
| | phenyl)methacrylamide | 2.36 (s, 3H), 1.94 (s, 3H). | |
| 409 | N-(4-(4-amino-5-(3- | ¹H NMR (400 MHz, DMSO) δ 10.71 (s, 1H), | 494 |
| | fluoro-4-((6-methyl- | 8.15 (s, 1H), 7.85-7.74 (m, 1H), 7.63-7.52 (m, | |
| | pyridin-2-yl)oxy) | 2H), 7.48-7.33 (m, 4H), 7.28-7.21 (m, 1H), | |
| | phenyl)pyrazolo[5,1- | 7.07-7.01 (m, 1H), 6.97-6.91 (m, 1H), 2.39- | |
| | f][1,2,4]triazin-6- | 2.30 (m, 3H), 2.10-1.90 (m, 3H). | |
| | yl)phenyl)but-2- | | |
| | ynamide | | |
| 410 | N-(4-(4-amino-5-(3- | ¹H NMR (400 MHz, DMSO) δ 10.92 (s, 1H), | 480 |
| | fluoro-4-((6-methyl- | 8.16 (s, 1H), 7.82-7.73 (m, 1H), 7.58 (d, J = 8.8 | |
| | pyridin-2-yl)oxy) | Hz, 2H), 7.49-7.35 (m, 4H), 7.25 (dd, J = 8.2, | |
| | phenyl)pyrazolo[5,1- | 1.6 Hz, 1H), 7.04 (d, J = 7.2 Hz, 1H), 6.94 (d, | |
| | f][1,2,4]triazin-6-yl) | J = 8.2 Hz, 1H), 4.45 (s, 1H), 2.35 (s, 3H). | |
| | phenyl)propiolamide | | |
| 411 | N-(4-(4-amino-5-(3- | ¹H NMR (400 MHz, DMSO) δ 10.21 (s, 1H), | 518 |
| | fluoro-4-((6-methyl- | 8.15 (s, 1H), 7.85-7.71 (m, 1H), 7.46-7.36 (m, | |
| | pyridin-2-yl)oxy) | 4H), 7.24 (dd, J = 8.0, 1.5 Hz, 1H), 7.13-7.09 | |
| | phenyl)pyrazolo[5,1-f] | (m, 2H), 7.05 (d, J = 7.2 Hz, 1H), 6.94 (d, J = 8.0 | |
| | [1,2,4]triazin-6- | Hz, 1H), 6.81 (dd, J = 16.4, 9.9 Hz, 1H), 6.14 (d, | |
| | yl)phenyl)ethenesulfonamide | J = 16.4 Hz, 1H), 6.05 (d, J = 10.0 Hz, 1H), 2.35 | |
| | | (s, 3H). | |
| 412 | (E)-N-(4-(4-amino-5- | ¹H NMR (400 MHz, DMSO) δ 10.15 (s, 1H), | 539 |
| | (3-fluoro-4-((6-methyl- | 8.16 (s, 1H), 7.83-7.73 (m, 1H), 7.67-7.58 (m, | |
| | pyridin-2-yl)oxy) | 2H), 7.48-7.35 (m, 4H), 7.29-7.22 (m, 1H), | |
| | phenyl)pyrazolo[5,1-f] | 7.08-7.01 (m, 1H), 6.97-6.91 (m, 1H), 6.78- | |
| | [1,2,4]triazin-6- | 6.69 (m, 1H), 6.32-6.23 (m, 1H), 3.09-3.02 | |
| | yl)phenyl)-4- | (m, 2H), 2.36 (s, 3H), 2.17 (s, 6H). | |
| | (dimethylamino)but-2- | | |
| | enamide | | |
| 413 | 1-(4-(4-amino-5-(3- | ¹H NMR (400 MHz, DMSO) δ 8.12 (s, 1H), 7.78 | 472 |
| | fluoro-4-((6-methyl- | (t, J = 7.8 Hz, 1H), 7.48 (d, J = 11.1 Hz, 1H), | |
| | pyridin-2-yl)oxy) | 7.41 (t, J = 8.3 Hz, 1H), 7.28 (d, J = 8.2 Hz, 1H), | |
| | phenyl)pyrazolo[5,1-f] | 7.04 (d, J = 7.3 Hz, 1H), 6.94 (d, J = 8.1 Hz, | |
| | [1,2,4]triazin-6-yl)-3,6- | 1H), 6.78 (m, 1H), 6.11 (m, 1H), 5.82 (d, J = | |
| | dihydropyridin-1(2H)- | 10.6 Hz, 1H), 5.68 (d, J = 10.3 Hz, 1H), 4.09 (m, | |
| | yl)prop-2-en-1-one | 2H), 3.68 (m, 2H), 2.41 (m, 2H), 2.33 (s, 3H). | |
| 414 | (E)-N-(4-(4-amino-5- | ¹H NMR (400 MHz, DMSO) δ 10.04 (s, 1H), | 496 |
| | (3-fluoro-4-((6-methyl- | 8.16 (s, 1H), 7.80-7.75 (m, 1H), 7.63 (d, J = 8.8 | |
| | pyridin-2-yl)oxy) | Hz, 2H), 7.47-7.37 (m, 4H), 7.25 (dd, J = 8.0, | |
| | phenyl)pyrazolo[5,1-f] | 1.2 Hz, 1H), 7.05 (d, J = 7.2 Hz, 1H), 6.94 (d, | |
| | [1,2,4]triazin-6- | J = 8.0 Hz, 1H), 6.80 (dq, J = 13.6, 6.8 Hz, 1H), | |
| | yl)phenyl)but-2- | 6.12 (dd, J = 15.2, 1.6 Hz, 1H), 2.36 (s, 3H), | |
| | enamide | 1.87 (dd, J = 6.8, 1.6 Hz, 3H). | |
| 415 | 4-(4-amino-6-(4-(2- | ¹H NMR (400 MHz, DMSO) δ 10.37 (s, 1H), | 586 |
| | fluoroacrylamido)-2- | 8.66 (s, 1H), 8.16 (s, 1H), 7.61 (d, J = 7.9 Hz, | |
| | methoxyphenyl)pyrazolo[5,1- | 1H), 7.46-7.39 (m, 2H), 7.29 (d, J = 8.2 Hz, | |
| | f][1,2,4]triazin-5- | 1H), 6.98 (d, J = 1.1 Hz, 1H), 6.86 (dd, J = 7.9, | |
| | yl)-2-methoxy-N-(1- | 1.3 Hz, 1H), 5.72 (dd, J = 47.6, 3.7 Hz, 1H), 5.45 | |
| | (trifluoromethyl)cyclo- | (dd, J = 15.6, 3.7 Hz, 1H), 3.72 (s, 3H), 3.37 (s, | |
| | propyl)benzamide | 3H), 1.34-1.26 (m, 2H), 1.18-1.09 (m, 2H). | |
| 416 | 4-(4-amino-6-(4-(2- | ¹H NMR (400 MHz, MeOD) δ 8.10 (s, 1H), 7.83 | 562 |
| | fluoroacrylamido)-2- | (d, J = 7.8 Hz, 1H), 7.41 (dd, J = 13.8, 5.0 Hz, | |
| | methoxyphenyl)pyrazolo[5,1- | 2H), 7.31 (dd, J = 8.4, 2.0 Hz, 1H), 7.02 (d, J = | |
| | f][1,2,4]triazin-5- | 7.8 Hz, 1H), 6.97 (d, J = 1.2 Hz, 1H), 5.72 (dd, | |
| | yl)-N-(3- | J = 46.4, 3.2 Hz, 1H), 5.31 (dd, J = 15.2, 3.2 Hz, | |
| | fluorobicyclo[1.1.1]pentan- | 1H), 3.75 (s, 3H), 3.38 (s, 3H), 2.44 (d, J = 2.0 | |
| | 1-yl)-2- | Hz, 6H). | |
| | methoxybenzamide | | |
| 417 | N-(4-(4-amino-5-(3- | ¹H NMR (400 MHz, DMSO) δ 10.24 (s, 1H), | 496 |
| | fluoro-4-(methyl(4- | 8.27-8.26 (d, J = 4.0 Hz, 1H), 8.17 (s, 1H), 7.67- | |
| | methylpyrimidin-2- | 7.64 (d, J = 12.0 Hz, 2H), 7.56-7.52 (t, J = 4.0 | |
| | yl)amino)phenyl)pyrazolo[5,1- | Hz, 1H), 7.44-7.41(d, J = 12.0 Hz, 2H), 7.36- | |
| | f][1,2,4]triazin- | 7.33 (m, 1H), 7.29-7.26 (m, 1H), 6.70-6.99 (d, | |
| | 6-yl)phenyl)acrylamide | J = 4.0 Hz, 1H), 6.47-6.40 (m, 10.1 Hz, 1H), 6.28- | |
| | | 6.23 (m, 1H), 5.78-5.75 (m, 1H), 3.48 (s, 3H), | |
| | | 2.31 (s, 3H). | |
| 418 | N-(4-(4-amino-5-(4- | ¹H NMR (400 MHz, DMSO) δ 10.25 (s, 1H), | 491 |
| | benzoyl-3- | 8.18 (s, 1H), 7.85-7.84 (d, J = 4.0 Hz, 2H), 7.68- | |
| | methoxyphenyl)pyrazolo[5,1- | 7.65 (d, J = 12.0 Hz, 3H), 7.58-7.54 (t, J = 8.0 | |
| | f][1,2,4]triazin-6- | Hz, 2H), 7.43-7.31 (m, 3H), 7.24 (s, 1H), 7.08- | |
| | yl)phenyl)acrylamide | 7.06 (d, 8.0 Hz, 1H), 6.47-6.41 (m, 1H), 6.29- | |
| | | 6.24 (m, 1H), 5.78-5.75 (m, 1H), 3.61 (s, 3H). | |
| 419 | N-(4-(4-amino-5-(4- | ¹H NMR (400 MHz, DMSO) δ 10.23 (s, 1H), | 533 |
| | (4,4- | 8.17 (s, 1H), 7.60 (m, 3H), 7.37 (d, J = 8.8 Hz, | |
| | difluorocyclohexane-1- | 2H), 7.23 (d, J = 1.2 Hz, 1H), 7.01 (dd, J = 7.8, | |
| | carbonyl)-3- | 1.2 Hz, 1H), 6.43 (m, 1H), 6.25 (m, 1H), 5.76 | |
| | methoxyphenyl)pyrazolo[5,1- | (m, 1H), 3.83 (s, 3H), 3.40 (m, 1H), 2.17-1.74 | |
| | f][1,2,4]triazin-6- | (m, 6H), 1.73-1.47 (m, 2H). | |
| | yl)phenyl)acrylamide | | |
| 420 | 1-(4-(4-amino-5-(3- | ¹H NMR (400 MHz, DMSO) δ 8.09 (s, 1H), 7.86- | 474 |
| | fluoro-4-((6- | 7.64 (m, 1H), 7.51-7.34 (m, 1H), 7.28 (d, J = | |
| | methylpyridin-2- | 8.3 Hz, 1H), 7.04 (d, J = 7.3 Hz, 1H), 6.93 (d, | |
| | yl)oxy)phenyl)pyrazolo[5,1- | J = 8.2 Hz, 1H), 6.86-6.72 (m, 1H), 6.12 (d, J = | |
| | f][1,2,4]triazin-6- | 2.5 Hz, 1H), 5.66 (d, J = 12.9 Hz, 1H), 4.46 (s, | |
| | yl)piperidin-1-yl)prop- | 1H), 4.12 (s, 1H), 3.08 (d, J = 31.6 Hz, 2H), 2.69 | |
| | 2-en-1-one | (d, J = 15.7 Hz, 2H), 2.34 (s, 3H), 2.29-2.18 (m, | |
| | | 1H), 1.83 (m, 2H), 1.64 (m, 2H). | |
| 421 | 1-(4-(4-(4-amino-5-(3- | ¹H NMR (400 MHz, DMSO) δ 8.11 (s, 1H), 7.87 | 540 |
| | fluoro-4-((6- | (s, 1H), 7.82-7.74 (m, 1H), 7.50 (m, 1H), 7.44 | |
| | methylpyridin-2- | (t, J = 8.3 Hz, 1H), 7.32 (s, 1H), 7.31 (d, J = 1.3 | |
| | yl)oxy)phenyl)pyrazolo | Hz, 1H), 7.04 (d, J = 7.3 Hz, 1H), 6.95 (d, J = | |
| | [5,1-f][1,2,4]triazin-6- | 8.2 Hz, 1H), 6.83 (m, 1H), 6.11 (m, 1H), 5.68 (m, | |
| | yl)-1H-pyrazol-1- | 1H), 4.46 (m, 2H), 4.13 (s, 1H), 3.20 (m, 2H), | |
| | yl)piperidin-1-yl)prop- | 2.80 (t, J = 12.2 Hz, 1H), 2.34 (s, 3H), 2.03 (d, | |
| | 2-en-1-one | J = 10.8 Hz, 2H), 1.86-1.69 (m, 2H). | |
| 422 | N-(4-(4-amino-5-(3- | ¹H NMR (400 MHz, DMSO) δ 11.24 (s, 1H), | 539 |
| | fluoro-4-((6- | 8.46 (s, 1H), 8.16 (s, 1H), 7.84-7.71 (m, 1H), | |
| | methylpyridin-2- | 7.64-7.55 (m, 2H), 7.47-7.42 (m, 1H), 7.41- | |
| | yl)oxy)phenyl)pyrazolo | 7.37 (m, 2H), 7.27-7.22 (m, 1H), 7.08-7.01 | |
| | [5,1-f][1,2,4]triazin-6- | (m, 1H), 6.97-6.92 (m, 1H), 6.03-5.99 (m, | |
| | yl)phenyl)-2- | 1H), 5.57 (s, 1H), 3.22 (s, 2H), 2.36 (s, 3H), 2.24 | |
| | ((dimethylami- | (s, 6H). | |
| | no)methyl)acrylamide | | |
| 423 | N-(4-(4-amino-5-(3- | ¹H NMR (400 MHz, DMSO) δ 10.42 (s, 1H), | 504 |
| | fluoro-4-((6-methyl- | 8.16 (s, 1H), 7.83-7.72 (m, 1H), 7.62-7.54 (m, | |
| | pyridin-2-yl)oxy) | 2H), 7.47-7.37 (m, 4H), 7.28-7.21 (m, 1H), | |
| | phenyl)pyrazolo[5,1- | 7.08-7.01 (m, 1H), 6.96-6.91 (m, 1H), 4.25 (s, | |
| | f][1,2,4]triazin-6-yl) | 2H), 2.36 (s, 3H). | |
| | phenyl)-2- | | |
| | chloroacetamide | | |
| 424 | N-(4-(4-amino-5-(3- | ¹H NMR (400 MHz, DMSO) δ 10.30 (s, 1H), | 516 |
| | fluoro-4-((6- | 8.17 (s, 1H), 7.78 (t, J = 8.0 Hz, 1H), 7.66 (d, J = | |
| | methylpyridin-2- | 8.8 Hz, 2H), 7.47-7.38 (m, 4H), 7.25 (dd, J = | |
| | yl)oxy)phenyl)pyrazolo | 8.4, 1.6 Hz, 1H), 7.04 (d, J = 7.2 Hz, 1H), 6.94 | |
| | [5,1-f][1,2,4]triazin-6- | (d, J = 8.4 Hz, 1H), 6.41 (d, J = 2.8 Hz, 1H), | |
| | yl)phenyl)-2- | 6.09 (d, J = 2.8 Hz, 1H), 2.36 (s, 3H). | |
| | chloroacrylamide | | |
| 425 | (Z)-4-((4-(4-amino-5- | ¹H NMR (400 MHz, DMSO) δ 10.46 (s, 1H), | 526 |
| | (3-fluoro-4-((6- | 8.16 (s, 1H), 7.81-7.74 (m, 1H), 7.61 (d, J = 8.8 | |
| | methylpyridin-2- | Hz, 2H), 7.47-7.38 (m, 4H), 7.25 (dd, J = 8.4, | |
| | yl)oxy)phenyl)pyrazolo | 1.6 Hz, 1H), 7.04 (d, J = 7.2 Hz, 1H), 6.94 (d, | |
| | [5,1-f][1,2,4]triazin-6- | J = 8.4 Hz, 1H), 6.48 (d, J = 12.0 Hz, 1H), 6.29 (d, | |
| | yl)phenyl)amino)-4- | J = 12.0 Hz, 1H), 2.35 (s, 3H). | |
| | oxobut-2-enoic acid | | |
| 426 | 4-(4-amino-6-(4-(2- | ¹H NMR (400 MHz, DMSO) δ 10.36 (s, 1H), | 544 |
| | fluoroacrylamido)-2- | 8.43 (s, 1H), 8.16 (s, 1H), 7.67 (d, J = 8.0 Hz, | |
| | methoxyphenyl)pyrazolo[5,1- | 1H), 7.42 (dd, J = 5.6, 2.0 Hz, 2H), 7.31 (d, J = | |
| | f][1,2,4]triazin-5- | 8.8 Hz, 1H), 6.96 (d, J = 1.2 Hz, 1H), 6.85 (dd, | |
| | yl)-N-(bicyclo[1.1.1] | J = 8.0, 1.6 Hz, 1H), 5.72 (dd, J = 47.6, 3.6 Hz, | |
| | pentan-1-yl)-2- | 1H), 5.45 (dd, J = 15.6, 3.6 Hz, 1H), 3.71 (s, | |
| | methoxybenzamide | 3H), 3.33 (s, 3H), 2.45 (s, 1H), 2.06 (s, 6H). | |
| 427 | 4-(4-amino-6-(2-fluoro- | ¹H NMR (400 MHz, DMSO) δ 10.57 (s, 1H), | 550 |
| | 4-(2-fluoroacryl- | 8.64 (s, 1H), 8.20 (s, 1H), 7.72-7.56 (m, 3H), | |
| | amido)phenyl)pyrazolo[5,1- | 7.44 (t, J = 8.4 Hz, 1H), 7.06 (d, J = 1.2 Hz, 1H), | |
| | f][1,2,4]triazin-5- | 6.90 (dd, J = 7.8, 1.2 Hz, 1H), 5.75 (dd, J = 47.6, | |
| | yl)-N-(3-fluorobicyclo | 3.8 Hz, 1H), 5.48 (dd, J = 15.6, 3.6 Hz, 1H), 3.74 | |
| | [1.1.1]pentan-1-yl)-2- | (s, 3H), 2.40 (d, J = 2.2 Hz, 6H). | |
| | methoxybenzamide | | |
| 428 | N-(4-(4-amino-5-(4-((4- | ¹H NMR (400 MHz, DMSO) δ 10.25 (s, 1H), | 484 |
| | aminopyrimidin-2- | 8.16 (s, 1H), 7.88 (d, J = 5.8 Hz, 1H), 7.66 (d, | |
| | yl)oxy)-3-fluoro- | J = 8.8 Hz, 2H), 7.43 (s, 2H), 7.40 (dd, J = 3.6, 2.0 | |
| | phenyl)pyrazolo[5,1-f] | Hz, 2H), 7.24 (dd, J = 8.4, 1.6 Hz, 1H), 7.11 (s, | |
| | [1,2,4]triazin-6- | 1H), 6.44 (dd, J = 17.2, 10.1 Hz, 1H), 6.28 (d, | |
| | yl)phenyl)acrylamide | J = 2.0 Hz, 1H), 6.25-6.19 (m, 2H), 5.77 (dd, J = | |
| | | 10.0, 2.0 Hz, 1H). | |
| 429 | 4-(4-amino-6-(2-fluoro- | ¹H NMR (400 MHz, DMSO) δ 10.06 (s, 1H), | 570 |
| | 4-methacrylamido- | 8.69 (s, 1H), 8.20 (s, 1H), 7.73-7.58 (m, 2H), | |
| | phenyl)pyrazolo[ | 7.57-7.46 (m, 1H), 7.44-7.28 (m, 1H), 7.08 (s, | |
| | 5,1-f][1,2,4]triazin-5- | 1H), 6.98-6.82 (m, 1H), 5.82 (s, 1H), 5.57 (s, | |
| | yl)-2-methoxy-N-(1- | 1H), 3.76 (s, 3H), 1.94 (s, 3H), 1.35-1.27 (m, | |
| | (trifluoromethyl)cyclo- | 2H), 1.20-1.12 (m, 2H). | |
| | propyl)benzamide | | |
| 430 | N-(4-(4-amino-7- | ¹H NMR (400 MHz, DMSO) δ 10.30 (s, 1H), | 519 |
| | cyano-3-(3-fluoro-4- | 8.21 (s, 1H), 7.78-7.67 (m, 3H), 7.31-7.20 (m, | |
| | ((6-methylpyridin-2- | 4H), 7.08 (dd, J = 8.2, 1.6 Hz, 1H), 7.00 (d, J = | |
| | yl)oxy)phenyl)-1- | 7.2 Hz, 1H), 6.86 (d, J = 8.2 Hz, 1H), 6.44 (dd, | |
| | methyl-1H-pyrrolo[3,2- | J = 17.0, 10.1 Hz, 1H), 6.27 (dd, J = 17.0, 2.0 Hz, | |
| | c]pyridin-2- | 1H), 5.78 (dd, J = 10.0, 2.0 Hz, 1H), 3.82 (s, | |
| | yl)phenyl)acrylamide | 3H), 2.28 (s, 3H). | |
| 431 | N-(4-(4-amino-5-(4- | ¹H NMR (400 MHz, DMSO) δ 10.22 (s, 1H), | 497 |
| | (cyclohexanecarbonyl)- | 8.16 (s, 1H), 7.62 (d, J = 8.8 Hz, 2H), 7.51 (d, | |
| | 3-methoxyphenyl) | J = 7.6 Hz, 1H), 7.37 (d, J = 8.8 Hz, 2H), 7.21 (d, | |
| | pyrazolo[5,1-f][1,2,4] | J = 1.2 Hz, 1H), 6.99 (m, 1H), 6.42 (m, 1H), 6.25 | |
| | triazin-6-yl)phenyl) | (m, 1H), 5.76 (m, 1H), 3.80 (s, 3H), 3.21 (m, | |
| | acrylamide | 1H), 1.86 (d, J = 7.6 Hz, 2H), 1.74 (m, 2H), 1.65 | |
| | | (m, 1H), 1.33 (m, 4H), 1.23 (m, 1H). | |
| 432 | 4-(4-amino-6-(4-(2- | ¹H NMR (400 MHz, DMSO) δ 10.67 (s, 1H), | 624 |
| | fluoroacrylamido)-2- | 8.64 (s, 1H), 8.26 (d, J = 2.0 Hz, 1H), 8.21 (s, | |
| | (trifluoromethyl)phe- | 1H), 7.98 (m, 1H), 7.59 (d, J = 7.6 Hz, 1H), 7.45 | |
| | nyl)pyrazolo[5,1-f] | (d, J = 8.4 Hz, 1H), 6.98 (m, 1H), 6.84 (dd, J = | |
| | [1,2,4]triazin-5-yl)-2- | 8.0, 1.2 Hz, 1H), 5.76 (m, 1H), 5.50 (m, 1H), | |
| | methoxy-N-(1- | 3.72 (s, 3H), 1.29 (m, 2H), 1.13 (m, 2H). | |
| | (trifluoromethyl)cyclo- | | |
| | propyl)benzamide | | |
| 433 | N-(4-(4-amino-5-(3- | ¹H NMR (400 MHz, DMSO) δ 10.25 (s, 1H), | 498 |
| | fluoro-4-((4- | 8.16 (s, 1H), 7.85 (s, 1H), 7.63 (d, J = 8.8 Hz, | |
| | (methylamino)pyrimidin- | 3H), 7.41 (m, 4H), 7.25 (d, J = 8.5 Hz, 1H), 6.43 | |
| | 2-yl)oxy)phenyl) | (m, 1H), 6.26 (m, 2H), 5.77 (m, 1H), 2.71 (d, J = | |
| | pyrazolo[5,1-f][1,2,4] | 4.2 Hz, 3H). | |
| | triazin-6-yl)phenyl) | | |
| | acrylamide | | |
| 434 | N-(4-(4-amino-5-(3- | ¹H NMR (400 MHz, DMSO) δ 10.08 (s, 1H), | 514 |
| | fluoro-4-((6-methyl- | 8.19 (s, 1H), 7.75 (t, J = 7.6 Hz, 1H), 7.66 (dd, | |
| | pyridin-2-yl)oxy) | J = 12.8, 1.6 Hz, 1H), 7.53 (dd, J = 8.4, 1.6 Hz, | |
| | phenyl)pyrazolo[5,1-f] | 1H), 7.39 (t, J = 8.4 Hz, 1H), 7.34-7.28 (m, | |
| | [1,2,4]triazin-6-yl)-3- | 2H), 7.12 (dd, J = 8.4, 1.2 Hz, 1H), 7.02 (d, J = | |
| | fluorophenyl) | 7.2 Hz, 1H), 6.88 (d, J = 8.0 Hz, 1H), 5.81 (s, | |
| | methacrylamide | 1H), 5.56 (s, 1H), 2.32 (s, 3H), 1.95 (s, 3H). | |
| 435 | N-(4-(4-amino-7- | ¹H NMR (400 MHz, DMSO) δ 10.31 (s, 1H), | 556 |
| | cyano-3-(4-((4- | 8.89 (d, J = 5.2 Hz, 1H), 8.21 (s, 1H), 7.73 (d, | |
| | (difluoromethyl)pyrimidin- | J = 8.4 Hz, 2H), 7.59 (d, J = 5.2 Hz, 1H), 7.44 (t, | |
| | 2-yl)oxy)-3- | J = 8.4 Hz, 1H), 7.32 (d, J = 8.4 Hz, 2H), 7.27 (s, | |
| | fluorophenyl)-1- | 1H), 7.15 (d, J = 8.8 Hz, 1H), 6.93 (t, J = 54.2 | |
| | methyl-1H-pyrrolo[3,2- | Hz, 1H), 6.44 (dd, J = 17.2, 10.2 Hz, 1H), 6.28 | |
| | c]pyridin-2-yl)phenyl) | (d, J = 17.2 Hz, 1H), 5.78 (d, J = 10.2 Hz, 1H). | |
| | acrylamide | | |
| 436 | N-(4-(4-amino-7- | ¹H NMR (400 MHz, DMSO) δ 10.31 (s, 1H), | 538 |
| | cyano-3-(3-fluoro-4- | 8.56 (d, J = 1.2 Hz, 1H), 8.21 (s, 1H), 7.73 (d, | |
| | ((5-fluoro-4-methyl- | J = 8.8 Hz, 2H), 7.37 (t, J = 8.4 Hz, 1H), 7.32 (d, | |
| | pyrimidin-2-yl)oxy) | J = 8.8 Hz, 2H), 7.25 (dd, J = 11.2, 1.9 Hz, 1H), | |
| | phenyl)-1-methyl-1H- | 7.12 (dd, J = 8.4, 1.2 Hz, 1H), 6.44 (dd, J = 17.2, | |
| | pyrrolo[3,2-c]pyridin-2- | 10.4 Hz, 1H), 6.27 (dd, J = 17.4, 2.0 Hz, 1H), | |
| | yl)phenyl)acrylamide | 5.78 (dd, J = 10.4, 2.0 Hz, 1H), 3.80 (s, 3H), | |
| | | 2.41 (d, J = 2.4 Hz, 3H). | |
| 437 | N-(4-(4-amino-7- | ¹H NMR (400 MHz, DMSO) δ 10.25 (s, 1H), | 501 |
| | cyano-1-methyl-3-(4- | 8.18 (s, 1H), 7.75 (t, J = 7.7 Hz, 1H), 7.68 (d, J = | |
| | ((6-methylpyridin-2- | 8.5 Hz, 2H), 7.33 (s, 2H), 7.22 (d, J = 8.4 Hz, | |
| | yl)oxy)phenyl)-1H- | 2H), 7.11 (d, J = 8.6 Hz, 2H), 7.04 (d, J = 7.3 | |
| | pyrrolo[3,2-c]pyridin-2- | Hz, 1H), 6.79 (d, J = 8.1 Hz, 1H), 6.47-6.38 (m, | |
| | yl)phenyl)acrylamide | 1H), 6.26 (m, 1H), 5.76 (m, 1H), 3.83 (s, 3H), | |
| | | 2.34 (s, 3H). | |
| 438 | N-(4-(4-amino-5-(3- | ¹H NMR (400 MHz, DMSO) δ 10.58 (s, 1H), | 518 |
| | fluoro-4-((6- | 8.20 (s, 1H), 7.79-7.72 (m, 1H), 7.68 (dd, J = | |
| | methylpyridin-2- | 12.4, 1.6 Hz, 1H), 7.62 (dd, J = 8.4, 2.0 Hz, 1H), | |
| | yl)oxy)phenyl)pyrazolo | 7.44 (t, J = 8.4 Hz, 1H), 7.31 (dd, J = 14.0, 5.2 | |
| | [5,1-f][1,2,4]triazin-6- | Hz, 2H), 7.12 (dd, J = 8.0, 1.2 Hz, 1H), 7.02 (d, | |
| | yl)-3-fluorophenyl)-2- | J = 7.2 Hz, 1H), 6.88 (d, J = 8.0 Hz, 1H), 5.75 (dd, | |
| | fluoroacrylamide | J = 47.6, 3.6 Hz, 1H), 5.48 (dd, J = 15.6, 3.6 Hz, | |
| | | 1H), 2.32 (s, 3H). | |
| 439 | N-(4-(4-amino-5-(3- | ¹H NMR (400 MHz, DMSO) δ 10.29 (s, 1H), | 514 |
| | fluoro-4-((6- | 8.18 (s, 1H), 7.78-7.71 (m, 1H), 7.62 (d, J = 1.6 | |
| | methylpyridin-2- | Hz, 1H), 7.60-7.56 (m, 1H), 7.28 (t, J = 8.4 Hz, | |
| | yl)oxy)phenyl)pyrazolo | 1H), 7.23 (dd, J = 11.2, 2.0 Hz, 1H), 7.20 (d, J = | |
| | [5,1-f][1,2,4]triazin-6- | 8.4 Hz, 1H), 7.06 (dd, J = 8.4, 1.2 Hz, 1H), 7.01 | |
| | yl)-3-methylphenyl)-2- | (d, J = 7.2 Hz, 1H), 6.87 (d, J = 8.4 Hz, 1H), | |
| | fluoroacrylamide | 5.71 (dd, J = 47.6, 3.6 Hz, 1H), 5.43 (dd, J = | |
| | | 15.6, 3.6 Hz, 1H), 2.30 (s, 3H), 2.01 (s, 3H). | |
| 440 | N-(4-(4-amino-7- | ¹H NMR (400 MHz, DMSO) δ 10.29 (s, 1H), | 508 |
| | cyano-3-(3-fluoro-4- | 8.19 (s, 1H), 7.70 (d, J = 8.4 Hz, 2H), 7.62 (d, | |
| | ((1-methyl-1H-pyrazol- | J = 2.4 Hz, 1H), 7.28 (d, J = 8.4 Hz, 2H), 7.22- | |
| | 3-yl)oxy)phenyl)-1- | 7.12 (m, 2H), 7.01 (dd, J = 8.4, 1.2 Hz, 1H), 6.44 | |
| | methyl-1H-pyrrolo[3,2- | (dd, J = 17.2, 10.0 Hz, 1H), 6.27 (dd, J = 17.2, | |
| | c]pyridin-2- | 2.0 Hz, 1H), 5.86 (d, J = 2.4 Hz, 1H), 5.78 (dd, | |
| | yl)phenyl)acrylamide | J = 10.0, 2.0 Hz, 1H), 3.79 (s, 3H), 3.72 (s, 3H). | |
| 441 | N-(4-(4-amino-5-(2-(1- | ¹H NMR (400 MHz, DMSO) 810.19 (s, 1H), | 468 |
| | methylcyclopropyl)ben- | 8.15 (s, 1H), 8.09 (s, 1H), 7.96 (d, J = 8.3 Hz, | |
| | zo[d]thiazol-6- | 1H), 7.57 (d, J = 8.7 Hz, 2H), 7.45 (dd, J = 8.3, | |
| | yl)pyrazolo[5,1- | 1.5 Hz, 1H), 7.35 (d, J = 8.7 Hz, 2H), 6.41 (dd, | |
| | f][1,2,4]triazin-6- | J = 17.0, 10.1 Hz, 1H), 6.23 (dd, J = 17.0, 1.8 Hz, | |
| | yl)phenyl)acrylamide | 1H), 5.74 (dd, J = 10.1, 1.9 Hz, 1H), 1.63 (s, | |
| | | 3H), 1.36 (q, J = 3.9 Hz, 2H), 1.16 (q, J = 3.9 | |
| | | Hz, 2H). | |
| 442 | N-(4-(4-amino-5-(3- | ¹H NMR (400 MHz, DMSO-d6) δ 10.20 (s, 1H), | 499 |
| | methoxy-4-((1- | 8.29 (s, 1H), 8.13 (s, 1H), 7.72-7.53 (m, 2H), | |
| | methylcyclohexyl)oxy) | 7.53-7.36 (m, 2H), 7.28-7.02 (m, 2H), 6.88 | |
| | phenyl)pyrazolo[5,1- | (dd, J = 8.1, 2.1 Hz, 1H), 6.42 (dd, J = 16.9, 10.1 | |
| | f][1,2,4]triazin-6- | Hz, 1H), 6.25 (dd, J = 17.0, 2.1 Hz, 1H), 5.75 | |
| | yl)phenyl)acrylamide | (dd, J = 10.0, 2.1 Hz, 1H), 5.49 (s, 1H), 3.68 (s, | |
| | | 3H), 1.89 (m, 2H), 1.71 (m, 2H), 1.63-1.40 (m, | |
| | | 5H), 1.35 (m, 1H), 1.23 (s, 3H) | |
| 443 | N-(4-(4-amino-7- | ¹H NMR (400 MHz, DMSO) δ10.33 (s, 1H), | 506 |
| | cyano-3-(3-fluoro-4- | 8.67 (d, J = 4.8 Hz, 2H), 8.36 (s, 1H), 7.75 (d, | |
| | (pyrimidin-2- | J = 8.4 Hz, 2H), 7.40 (t, J = 8.4 Hz, 1H), 7.36- | |
| | yloxy)phenyl)-1- | 7.31 (m, 3H), 7.28 (dd, J = 11.2, 2.0 Hz, 1H), | |
| | methyl-1H-pyrrolo[3,2- | 7.13 (dd, J = 8.4, 1.2 Hz, 1H), 6.44 (dd, J = 18.0, | |
| | c]pyridin-2- | 10.0 Hz, 1H), 6.28 (dd, J = 18.0, 2.0 Hz, 3H), | |
| | yl)phenyl)acrylamide | 5.79 (dd, J = 10.0, 2.0 Hz, 1H), 3.83 (s, 3H). | |
| 444 | N-(4-(4-amino-7- | ¹H NMR (400 MHz, DMSO) δ 10.31 (s, 1H), | 538 |
| | cyano-3-(4-((4- | 8.87-8.86 (d, J = 4.0 Hz, 1H), 8.37 (s, 1H), 7.74- | |
| | (difluoromethyl)pyr- | 7.71 (d, J = 12.0 Hz, 2H), 7.56-7.54 (d, J = 8.0 | |
| | imidin-2-yl)oxy)phenyl)-1- | Hz, 1H), 7.35-7.28 (m, 6H), 7.05-6.78(m, 1H), | |
| | methyl-1H-pyrrolo[3,2- | 6.47-6.40 (m, 1H), 6.30-6.25 (m, 1H), 5.80-5.77 | |
| | c]pyridin-2- | (m, 1H), 3.85 (s, 3H). | |
| | yl)phenyl)acrylamide | | |
| 445 | 4-(4-amino-6-(2-chloro- | ¹H NMR (400 MHz, DMSO) δ 10.75 (s, 1H), | 532 |
| | 4-(2-fluoroacrylamido) | 8.47 (d, J = 5.2 Hz, 1H), 8.20 (s, 1H), 7.64 (d, | |
| | phenyl)pyrazolo[5,1- | J = 8.4 Hz, 2H), 7.35 (t, J = 16.8 Hz, 1H), 7.30 (d, | |
| | f][1,2,4]triazin-5-yl)-2- | J = 8.4 Hz, 2H), 7.23 (d, J = 11.2 Hz, 1H), 7.18 | |
| | methoxy-N-(1- | (d, J = 5.2 Hz, 1H), 7.11 (d, J = 8 Hz, 1H), 3.79 | |
| | (trifluoromethyl)cyclo- | (s, 3H), 2.41 (s, 3H), 2.05 (s, 3H). | |
| | propyl)benzamide | | |
| 446 | 4-(4-amino-6-(4-(2- | ¹H NMR (400 MHz, DMSO) δ 10.29 (s, 1H), | 570 |
| | fluoroacrylamido)-2- | 8.66 (s, 1H), 8.19 (s, 1H), 7.62-7.60 (d, J = 8.0 | |
| | methylphenyl)pyrazolo[5,1- | Hz, 2H), 7.54-7.52 (dd, J = 8.0 Hz, 1H), 7.19- | |
| | f][1,2,4]triazin-5- | 7.17(d, J = 8.0 Hz, 1H), 6.98 (m, 1H), 6.88-6.85 | |
| | yl)-2-methoxy-N-(1- | (dd, J = 4.0 Hz, 1H), 5.77-5.64 (m, 1H), 5.46- | |
| | (trifluoromethyl)cyclo- | 5.41 (m, 1H), 3.70 (s, 3H), 2.00 (s, 3H), 1.31- | |
| | propyl)benzamide | 1.28 (m, 2H), 1.14 (s, 2H). | |
| 447 | 4-(4-amino-6-(2-chloro- | ¹H NMR (400 MHz, DMSO) δ 10.53 (s, 1H), | 590 |
| | 4-(2-fluoroacrylamido) | 8.66 (s, 1H), 8.22 (s, 1H), 7.93 (d, J = 2.0 Hz, | |
| | phenyl)pyrazolo[5,1- | 1H), 7.72 (dd, J = 8.4, 2.0 Hz, 1H), 7.61 (d, J = 8 | |
| | f][1,2,4]triazin-5-yl)-2- | Hz, 1H), 7.44 (d, J = 8.4 Hz, 1H), 7.00 (d, J = | |
| | methoxy-N-(1-(trifl4- | 1.2 Hz, 1H), 6.87 (dd, J = 9.2, 1.3 Hz, 1H), 5.75 | |
| | (4-amino-6-(2-chloro-4- | (dd, J = 51.2, 3.7 Hz, 1H), 5.48 (dd, J = 19.2, 3.7 | |
| | (2-fluoroacrylamido) | Hz, 1H), 3.72 (s, 3H), 1.29 (m, 2H), 1.14 (m, | |
| | phenyl)pyrazolo[5,1- | 2H). | |
| | f][1,2,4]triazin-5-yl)-2- | | |
| | methoxy-N-(1- | | |
| | (trifluoromethyl)cyclo- | | |
| | propyl)benzamide | | |
| 448 | N-(4-(4-amino-5-(3- | ¹H NMR (400 MHz, DMSO) δ10.54 (s, 1H), | 534 |
| | fluoro-4-((6- | 8.21 (s, 1H), 7.96 (d, J = 2.0 Hz, 1H), 7.80-7.69 | |
| | methylpyridin-2- | (m, 2H), 7.44 (d, J = 8.5 Hz, 1H), 7.32-7.20 (m, | |
| | yl)oxy)phenyl)pyrazolo | 2H), 7.07 (s, 1H), 7.01 (d, J = 7.3 Hz, 1H), 6.85 | |
| | [5,1-f][1,2,4]triazin-6- | (d, J = 8.2 Hz, 1H), 5.80 (s, 1H), 5.68 (s, 0H), | |
| | yl)-3-chlorophenyl)-2- | 5.47 (s, 1H), 2.29 (s, 3H). | |
| | fluoroacrylamide | | |
| 449 | N-(4-(4-amino-5-(3- | ¹H NMR (400 MHz, DMSO) δ 9.87 (s, 1H), 8.48 | 527 |
| | fluoro-4-((4-methyl- | (d, J = 5.0 Hz, 1H), 8.15 (s, 1H), 7.47-7.30 (m, | |
| | pyrimidin-2- | 4H), 7.20 (dd, J = 12.9, 3.4 Hz, 2H), 7.05 (d, J = | |
| | yl)oxy)phenyl)pyrazolo | 8.2 Hz, 1H), 5.81 (s, 1H), 5.53 (s, 1H), 3.37 (s, | |
| | [5,1-f][1,2,4]triazin-6- | 3H), 2.40 (s, 3H), 1.95 (s, 3H). | |
| | yl)-3-methoxyphenyl) | | |
| | methacrylamide | | |
| 450 | N-(4-(4-amino-5-(3- | ¹H NMR (400 MHz, DMSO) δ 9.79 (s, 1H), | 511 |
| | fluoro-4-((4-methyl- | 8.49-8.47 (d, J = 8.0 Hz, 1H), 8.18 (s, 1H), 7.58- | |
| | pyrimidin-2-yl)oxy) | 7.53 (m, 2H), 7.39-7.34 (t, J = 12.0 Hz, 1H), | |
| | phenyl)pyrazolo[5,1- | 7.27-7.24 (m, 1H), 7.19-7.16 (m, 2H), 7.11- | |
| | f][1,2,4]triazin-6-yl)-3- | 7.08 (d, J = 12.0 Hz, 1H), 5.80 (s, 1H), 5.51 (s, | |
| | methylphenyl)methacrylamide | 1H), 2.41 (s, 3H), 2.02 (s, 3H), 1.94 (s, 3H). | |
| 451 | N-(4-(4-amino-5-(3- | ¹H NMR (400 MHz, DMSO) δ 10.05 (s, 1H), | 531 |
| | fluoro-4-((4-methyl- | 8.47 (d, J = 4.8 Hz, 1H), 8.21 (s, 1H), 7.94 (s, | |
| | pyrimidin-2-yl)oxy) | 1H), 7.69 (d, J = 6.8 Hz, 1H), 7.39 (m, 2H), 7.26 | |
| | phenyl)pyrazolo[5,1- | (d, J = 11.2 Hz, 1H), 7.19 (d, J = 5.2 Hz, 1H), | |
| | f][1,2,4]triazin-6-yl)-3- | 7.10 (d, J = 8.4 Hz, 1H), 5.83 (s, 1H), 5.57 (s, | |
| | chlorophenyl)methacrylamide | 1H), 2.41 (s, 3H), 1.95 (s, 3H). | |
| 452 | N-(4-(4-amino-5-(3- | ¹H NMR (400 MHz, DMSO) δ 9.85 (s, 1H), 8.14 | 485 |
| | fluoro-4-((1-methyl- | (d, J = 2.4 Hz, 1H), 7.69-7.65 (m, 2H), 7.64 (d, | |
| | 1H-pyrazol-3-yl)oxy) | J = 3.6 Hz, 1H), 7.40 (dd, J = 11.6, 2.0 Hz, 1H), | |
| | phenyl)pyrazolo[5,1-f] | 7.36 (s, 1H), 7.33 (d, J = 4.8 Hz, 1H), 7.28 (t, J = | |
| | [1,2,4]triazin-6-yl) | 8.4 Hz, 1H), 7.18-7.14 (m, 1H), 5.92 (t, J = 9.2 | |
| | phenyl)methacrylamide | Hz, 1H), 5.79 (s, 1H), 5.52 (s, 1H), 3.75 (d, J = | |
| | | 9.2 Hz, 3H), 1.94 (s, 3H). | |
| 453 | N-(4-(4-amino-5-(3- | ¹H NMR (400 MHz, DMSO) δ 10.07 (s, 1H), | 503 |
| | fluoro-4-((1-methyl- | 8.17 (s, 1H), 7.67-7.61 (m, 2H), 7.52 (dd, J = | |
| | 1H-pyrazol-3-yl)oxy) | 8.4, 2.0 Hz, 1H), 7.37 (t, J = 8.4 Hz, 1H), 7.27 | |
| | phenyl)pyrazolo[5,1- | (dd, J = 11.6, 2.0 Hz, 1H), 7.20 (t, J = 8.4 Hz, | |
| | f][1,2,4]triazin-6-yl)-3- | 1H), 7.03 (dd, J = 8.4, 1.2 Hz, 1H), 5.88 (d, J = | |
| | fluorophenyl)methacrylamide | 2.4 Hz, 1H), 5.82 (s, 1H), 5.57 (s, 1H), 3.74 (s, | |
| | | 3H), 1.95 (s, 3H). | |
| 454 | N-(4-(4-amino-5-(3- | ¹H NMR (400 MHz, DMSO) δ 10.70 (s, 1H), | 483 |
| | fluoro-4-((1-methyl- | 8.14 (s, 1H), 7.67 (d, J = 2.4 Hz, 1H), 7.55 (d, | |
| | 1H-pyrazol-3-yl)oxy) | J = 8.4 Hz, 2H), 7.40 (dd, J = 11.2, 2.0 Hz, 1H), | |
| | phenyl)pyrazolo[5,1- | 7.35 (d, J = 8.4 Hz, 2H), 7.28 (t, J = 8.4 Hz, 1H), | |
| | f][1,2,4]triazin-6- | 7.15 (d, J = 9.2 Hz, 1H), 5.94 (d, J = 2.4 Hz, | |
| | yl)phenyl)but-2- | 1H), 3.76 (s, 3H), 2.04 (s, 3H). | |
| | ynamide | | |
| 455 | N-(4-(4-amino-5-(3- | ¹H NMR (400 MHz, DMSO) δ 10.90 (s, 1H), | 469 |
| | fluoro-4-((1-methyl- | 8.14 (s, 1H), 7.67 (d, J = 2.4 Hz, 1H), 7.56 (d, | |
| | 1H-pyrazol-3- | J = 8.4 Hz, 2H), 7.41 (d, J = 13.2 Hz, 1H), 7.37 (d, | |
| | yl)oxy)phenyl)pyrazolo | J = 8.4 Hz, 2H), 7.28 (t, J = 8.4 Hz, 1H), 7.15 (d, | |
| | [5,1-f][1,2,4]triazin-6- | J = 8.4 Hz, 1H), 5.94 (d, J = 2.0 Hz, 1H), 4.44 (s, | |
| | yl)phenyl)propiolamide | 1H), 3.76 (s, 3H). | |
| 456 | N-(4-(4-amino-5-(3- | ¹H NMR (400 MHz, DMSO) δ 10.24 (s, 1H), | 495 |
| | methoxy-4-(1-methyl- | 8.18 (s, 1H), 7.65 (d, J = 8.8 Hz, 2H), 7.58 (s, | |
| | 1H-imidazole-2- | 1H), 7.51 (d, J = 7.6 Hz, 1H), 7.45 (d, J = 8.8 | |
| | carbonyl)phenyl)pyrazolo[5,1- | Hz, 2H), 7.21 (d, J = 1.2 Hz, 1H), 7.14 (s, 1H), | |
| | f][1,2,4]triazin- | 7.04 (m, 1H), 6.43 (m, 1H), 6.26 (m, 1H), 5.76 | |
| | 6-yl)phenyl)acrylamide | (m, 1H), 4.03 (s, 3H), 3.66 (s, 3H). | |
| 457 | N-(4-(4-amino-7- | ¹H NMR (400 MHz, DMSO) δ 10.27 (s, 1H), | 490 |
| | cyano-1-methyl-3-(4- | 8.18 (s, 1H), 7.68 (d, J = 8.5 Hz, 2H), 7.65 (d, | |
| | ((1-methyl-1H-pyrazol- | J = 2.3 Hz, 1H), 7.26 (d, J = 8.6 Hz, 2H), 7.21 (d, | |
| | 3-yl)oxy)phenyl)-1H- | J = 8.7 Hz, 2H), 7.03 (d, J = 8.6 Hz, 2H), 6.41 | |
| | pyrrolo[3,2-c]pyridin-2- | (d, J = 10.1 Hz, 1H), 6.29 (d, J = 1.9 Hz, 1H), | |
| | yl)phenyl)acrylamide | 5.88 (d, J = 2.3 Hz, 1H), 5.77 (dd, J = 10.0, 2.0 | |
| | | Hz, 1H), 3.80 (s, 3H), 3.74 (s, 3H). | |
| 458 | N-(4-(4-amino-7- | ¹H NMR (400 MHz, DMSO) δ 10.13 (s, 1H), | 538 |
| | cyano-3-(4-((5- | 8.88-8.67 (m, 2H), 8.21 (s, 1H), 7.77 (m, 1H), | |
| | fluoropyrimidin-2- | 7.51 (m, 1H), 7.36-7.26 (m, 3H), 7.23 (m, 2H), | |
| | yl)oxy)phenyl)-1- | 5.81 (s, 1H), 5.58 (s, 1H), 3.75 (s, 3H), 1.94 (s, | |
| | methyl-1H-pyrrolo[3,2- | 3H). | |
| | c]pyridin-2-yl)-3- | | |
| | fluorophenyl)methacrylamide | | |
| 460 | N-(4-(4-amino-5-(3- | ¹H NMR (400 MHz, DMSO) δ 10.35 (s, 1H), | 530 |
| | fluoro-4-((6- | 8.15 (s, 1H), 7.80-7.70 (m, 1H), 7.46 (ddd, J = | |
| | methylpyridin-2- | 28.4, 12.0, 5.2 Hz, 3H), 7.28 (t, J = 8.4 Hz, 1H), | |
| | yl)oxy)phenyl)pyrazolo | 7.18 (dd, J = 11.6, 2.0 Hz, 1H), 7.01 (t, J = 8.0 | |
| | [5,1-f][1,2,4]triazin-6- | Hz, 2H), 6.89 (d, J = 8.0 Hz, 1H), 5.72 (dd, J = | |
| | yl)-3-methoxyphenyl)- | 47.6, 3.6 Hz, 1H), 5.44 (dd, J = 15.6, 3.6 Hz, | |
| | 2-fluoroacrylamide | 1H), 3.36 (s, 3H), 2.27 (s, 3H). | |
| 461 | N-(4-(4-amino-7- | ¹H NMR (400 MHz, DMSO) δ 10.14 (s, 1H), | 588 |
| | cyano-3-(4-((4- | 8.89 (d, J = 4.8 Hz, 1H), 8.23 (s, 1H), 7.79 (dd, | |
| | (difluoromethyl)pyr- | J = 12.4, 1.6 Hz, 1H), 7.59 (d, J = 5.2 Hz, 1H), | |
| | imidin-2-yl)oxy)-3- | 7.54 (dd, J = 8.4, 2.0 Hz, 1H), 7.44 (t, J = 8.4 Hz, | |
| | fluorophenyl)-1- | 1H), 7.34 (t, J = 8.4 Hz, 1H), 7.27 (d, J = 11.2 | |
| | methyl-1H-pyrrolo[3,2- | Hz, 1H), 7.13 (d, J = 8.4 Hz, 1H), 6.92 (t, J = | |
| | c]pyridin-2-yl)-3- | 54.0 Hz, 1H), 5.82 (s, 1H), 5.58 (s, 1H), 3.76 (s, | |
| | fluorophenyl)methacrylamide | 3H), 1.95 (s, 3H). | |
| 462 | N-(4-(4-amino-7- | ¹H NMR (400 MHz, DMSO) δ 10.14 (s, 1H), | 570 |
| | cyano-3-(3-fluoro-4- | 8.56 (d, J = 1.2 Hz, 1H), 8.23 (s, 1H), 7.78 (dd, | |
| | ((5-fluoro-4-methyl- | J = 12.4, 2.0 Hz, 1H), 7.53 (dd, J = 8.4, 2.0 Hz, | |
| | pyrimidin-2-yl)oxy) | 1H), 7.40-7.30 (m, 2H), 7.24 (d, J = 10.8 Hz, | |
| | phenyl)-1-methyl-1H- | 1H), 7.10 (d, J = 8.4 Hz, 1H), 5.82 (s, 1H), 5.58 | |
| | pyrrolo[3,2-c]pyridin-2- | (s, 1H), 3.75 (s, 3H), 2.40 (d, J = 2.4 Hz, 3H), | |
| | yl)-3-fluorophenyl) | 1.95 (s, 3H). | |
| | methacrylamide | | |
| 463 | N-(4-(4-amino-7-cyano- | ¹H NMR (400 MHz, DMSO) δ 10.15 (s, 1H), | 538 |
| | 3-(3-fluoro-4- | 8.68 (d, J = 4.8 Hz, 2H), 8.23 (s, 1H), 7.79 (dd, | |
| | (pyrimidin-2-yloxy) | J = 12.4, 1.6 Hz, 1H), 7.54 (dd, J = 8.4, 2.0 Hz, | |
| | phenyl)-1-methyl-1H- | 1H), 7.44-7.29 (m, 3H), 7.25 (d, J = 11.2 Hz, | |
| | pyrrolo[3,2-c] pyridin- | 1H), 7.12 (d, J = 8.4 Hz, 1H), 5.83 (s, 1H), 5.58 | |
| | 2-yl)-3-fluorophenyl) | (s, 1H), 3.75 (s, 3H), 1.95 (s, 3H). | |
| | methacrylamide | | |
| 464 | N-(4-(4-amino-7- | ¹H NMR (400 MHz, DMSO) δ 10.13 (s, 1H), | 566 |
| | cyano-3-(4-((4- | 8.50 (d, J = 5.2 Hz, 1H), 8.23 (s, 1H), 7.78 (m, | |
| | ethylpyrimidin-2- | 1H), 7.53 (m, 1H), 7.35 (m, 2H), 7.22 (m, 1H), | |
| | yl)oxy)-3-fluorophenyl)- | 7.18 (d, J = 5.2 Hz, 1H), 7.10 (m, 1H), 5.81 (s, | |
| | 1-methyl-1H-pyrrolo | 1H), 5.58 (s, 1H), 3.76 (s, 3H), 2.68 (m, 3H), | |
| | [3,2-c] pyridin-2-yl)-3- | 1.95 (s, 3H), 1.12 (m, 3H). | |
| | fluorophenyl)methacryl | | |
| | amide | | |
| 465 | N-(4-(4-amino-7- | ¹H NMR (400 MHz, DMSO) δ 10.16 (s, 1H), | 551 |
| | cyano-3-(3-fluoro-4- | 8.36 (s, 1H), 7.79 (dd, J = 12.4, 1.6 Hz, 1H), | |
| | ((6-methylpyridin-2- | 7.76-7.71 (m, 1H), 7.54 (dd, J = 8.4, 1.6 Hz, | |
| | yl)oxy)phenyl)-1- | 1H), 7.30 (dt, J = 10.8, 8.4 Hz, 2H), 7.21 (d, J = | |
| | methyl-1H-pyrrolo[3,2- | 10.4 Hz, 1H), 7.06 (d, J = 8.0 Hz, 1H), 7.00 (d, | |
| | c]pyridin-2-yl)-3- | J = 7.2 Hz, 1H), 6.86 (d, J = 8.4 Hz, 1H), 5.81 (s, | |
| | fluorophenyl)methacrylamide | 1H), 5.58 (s, 1H), 3.81 (s, 3H), 2.27 (s, 3H), 1.95 | |
| | | (s, 3H). | |
| 466 | N-(4-(4-amino-7-cyano- | ¹H NMR (400 MHz, DMSO) δ 10.14 (s, 1H), | 537 |
| | 3-(3-fluoro-4-(pyridin- | 8.23 (s, 1H), 8.14 (m, 1H), 7.93-7.83 (m, 1H), | |
| | 2-yloxy)phenyl)-1- | 7.79 (dd, J = 12.4, 1.9 Hz, 1H), 7.53 (m, 1H), | |
| | methyl-1H-pyrrolo[3,2- | 7.32 (m, 2H), 7.21 (d, J = 11.2 Hz, 1H), 7.18- | |
| | c]pyridin-2-yl)-3- | 7.06 (m, 3H), 5.82 (s, 1H), 5.58 (s, 1H), 3.75 (s, | |
| | fluorophenyl)methacrylamide | 3H), 1.95 (s, 3H). | |
| 467 | N-(4-(4-amino-7- | ¹H NMR (400 MHz, DMSO) δ 10.12 (s, 1H), | 534 |
| | cyano-1-methyl-3-(4- | 8.46-8.45 (d, J = 4.0 Hz, 1H), 8.21 (s, 1H), 7.79- | |
| | ((4-methylpyrimidin-2- | 7.75 (m, 1H), 7.52-7.49 (m, 1H), 7.32-7.28 (t, | |
| | yl)oxy)phenyl)-1H- | J = 8.0 Hz, 3H), 7.21-7.18 (d, J = 12.0 Hz, 2H), | |
| | pyrrolo[3,2-c]pyridin-2- | 7.15-7.14 (d, J = 4.0 Hz, 1H), 5.81 (s, 1H), 5.57 | |
| | yl)-3-fluorophenyl) | (s, 1H), 3.75 (s, 3H), 2.40 (s, 3H), 1.94 (s, 3H). | |
| | methacrylamide | | |
| 468 | N-(4-(4-amino-7- | ¹H NMR (400 MHz, DMSO) δ 10.12 (s, 1H), | 533 |
| | cyano-1-methyl-3-(4- | 8.20 (s, 1H), 7.79-7.70 (m, 2H), 7.50 (m, 1H), | |
| | ((6-methylpyridin-2- | 7.27 (m, 3H), 7.09 (d, J = 8.7 Hz, 2H), 7.01 (d, | |
| | yl)oxy)phenyl)-1H- | J = 7.4 Hz, 1H), 6.79 (s, 1H), 5.81 (s, 1H), 5.57 (s, | |
| | pyrrolo[3,2-c]pyridin-2- | 1H), 3.76 (s, 3H), 2.32 (s, 3H), 1.94 (s, 3H). | |
| | yl)-3-fluorophenyl) | | |
| | methacrylamide | | |
| 469 | N-(4-(4-amino-7- | ¹H NMR (400 MHz, DMSO) δ 10.14 (s, 1H), | 520 |
| | cyano-1-methyl-3-(4- | 8.66 (d, J = 4.8 Hz, 2H), 8.21 (s, 1H), 7.78 (dd, | |
| | (pyrimidin-2- | J = 12.4, 2.0 Hz, 1H), 7.51 (m, 1H), 7.34-7.27 | |
| | yloxy)phenyl)-1H- | (m, 4H), 7.24-7.22 (m, 2H), 5.82 (s, 1H), 5.58 | |
| | pyrrolo[3,2-c]pyridin-2- | (s, 1H), 3.75 (s, 3H), 1.94 (s, 3H). | |
| | yl)-3-fluorophenyl) | | |
| | methacrylamide | | |
| 470 | 4-(4-amino-7-cyano-2- | ¹H NMR (400 MHz, DMSO) δ 10.12 (s, 1H), | 581 |
| | (2-fluoro-4-methacrylami- | 8.65 (t, J = 12.8 Hz, 1H), 8.23 (s, 1H), 7.76 (d, | |
| | dophenyl)-1-methyl- | J = 10.0 Hz, 1H), 7.69 (d, J = 7.6 Hz, 1H), 7.50 (d, | |
| | 1H-pyrrolo[3,2-c] | J = 8.0 Hz, 1H), 7.31 (t, J = 16.8 Hz, 1H), 6.97 | |
| | pyridine-3-yl)-2- | (s, 1H), 6.89 (d, J = 7.6 Hz, 1H), 5.82 (s, 1H), | |
| | methoxy-N-(2,2,2-tri- | 5.58 (s, 1H), 4.08 (m, 2H), 3.76 (d, J = 2.8 Hz, | |
| | fluoroethyl)benzamide | 6H), 1.94 (s, 3H). | |
| 471 | N-(4-(4-amino-3-(3- | ¹H NMR (400 MHz, DMSO) δ 10.15 (s, 1H), | 568 |
| | chloro-4-((4-methyl- | 8.46 (d, J = 4.8 Hz, 1H), 8.23 (s, 1H), 7.79 (d, | |
| | pyrimidin-2-yl)oxy) | J = 12.4 Hz, 1H), 7.54 (d, J = 8.4 Hz, 1H), 7.43 (s, | |
| | phenyl)-7-cyano-1- | 1H), 7.35 (t, J = 18.0 Hz, 2H), 7.25 (d, J = 7.6 | |
| | methyl-1H-pyrrolo[3,2- | Hz, 1H), 7.17 (d, J = 4.8 Hz, 1H), 5.82 (s, 1H), | |
| | c]pyridin-2-yl)-3- | 5.58 (s, 1H), 3.75 (s, 3H), 2.40 (s, 3H), 1.95 (s, | |
| | fluorophenyl)methacrylamide | 3H). | |
| 472 | N-(4-(4-amino-7- | ¹H NMR (400 MHz, DMSO-d6) δ 10.13 (s, 1H), | 548 |
| | cyano-1-methyl-3-(3- | 8.43 (d, J = 5.0 Hz, 1H), 8.21 (s, 1H), 7.78 (dd, | |
| | methyl-4-((4-methyl- | J = 12.3, 1.8 Hz, 1H), 7.51 (dd, J = 8.5, 1.8 Hz, | |
| | pyrimidin-2-yl)oxy) | 1H), 7.33 (d, J = 8.5 Hz, 1H), 7.22 (s, 1H), 7.17- | |
| | phenyl)-1H-pyrrolo | 7.04 (m, 3H), 5.81 (s, 1H), 5.58 (s, 1H), 3.74 (s, | |
| | [3,2-c]pyridin-2-yl)-3- | 3H), 2.39 (s, 3H), 2.03 (s, 3H), 1.94 (s, 3H). | |
| | fluorophenyl) | | |
| | methacrylamide | | |
| 473 | N-(4-(4-amino-7-cyano- | ¹H NMR (400 MHz, DMSO) δ 10.11 (s, 1H), | 568 |
| | 3-(3-fluoro-4-((4- | 8.46 (d, J = 5.0 Hz, 1H), 8.31 (s, 1H), 8.05 (s, | |
| | methylpyrimidin-2- | 1H), 7.71 (d, J = 8.6 Hz, 1H), 7.42-7.31 (m, | |
| | yl)oxy)phenyl)-1- | 2H), 7.19 (t, J = 11.3 Hz, 2H), 7.09 (s, 1H), 5.83 | |
| | methyl-1H-pyrrolo[3,2- | (s, 1H), 5.58 (s, 1H), 3.71 (s, 3H), 2.39 (s, 3H), | |
| | c]pyridin-2-yl)-3- | 1.94 (s, 3H). | |
| | chlorophenyl)methacrylamide | | |
| 475 | N-(4-(4-amino-7-cyano- | ¹H NMR (400 MHz, DMSO) δ 10.17 (s, 1H), | 570 |
| | 3-(3,5-difluoro-4-((4- | 8.50 (d, J = 5.2 Hz, 1H), 8.24 (s, 1H), 7.80 (d, | |
| | methylpyrimidin-2-yl) | J = 12.4 Hz, 1H), 7.57 (d, J = 8.4 Hz, 1H), 7.36 (t, | |
| | oxy)phenyl)-1-methyl- | J = 8.4 Hz, 1H), 7.24 (d, J = 5.2 Hz, 1H), 7.10 | |
| | 1H-pyrrolo[3,2-c] | (d, J = 8.4 Hz, 2H), 5.83 (s, 1H), 5.59 (s, 1H), | |
| | pyridin-2-yl)-3- | 3.75 (s, 3H), 2.42 (s, 3H), 1.95 (s, 3H). | |
| | fluorophenyl)methacrylamide | | |
| 476 | N-(4-(4-amino-7-cyano- | ¹H NMR (400 MHz, DMSO) δ 10.15 (s, 1H), | 566 |
| | 3-(4-((4,5-dimethyl- | 8.28 (s, 1H), 8.23 (s, 1H), 7.79 (m, 1H), 7.54 (m, | |
| | pyrimidin-2-yl)oxy)-3- | 1H), 7.33 (m, 2H), 7.21 (d, J = 11.2 Hz, 1H), | |
| | fluorophenyl)-1-methyl- | 7.09 (d, J = 8.0 Hz, 1H), 5.82 (s, 1H), 5.58 (s, | |
| | 1H-pyrrolo [3,2-c] | 1H), 3.75 (s, 3H), 2.35 (s, 3H), 2.16 (s, 3H), 1.95 | |
| | pyridin-2-yl)-3- | (s, 3H). | |
| | fluorophenyl) | | |
| | methacrylamide | | |
| 477 | N-(4-(4-amino-7- | ¹H NMR (400 MHz, DMSO) δ 10.31 (s, 1H), | 524 |
| | cyano-3-(3-fluoro-4- | 8.77 (s, 2H), 8.21 (s, 1H), 7.74-7.72 (d, J = 8.0 | |
| | ((5-fluoropyrimidin-2- | Hz, 2H), 7.40-7.38 (t, J = 8.0 Hz, 1H), 7.34-7.32 | |
| | yl)oxy)phenyl)-1- | (d, J = 8.0 Hz, 2H), 7.28-7.25 (m, 1H), 7.15-7.13 | |
| | methyl-1H-pyrrolo[3,2- | (d, J = 8.0 Hz, 1H), 6.48-6.41 (m, 1H), 6.30-6.25 | |
| | c]pyridin-2- | (m, 1H), 5.80-5.77 (m, 1H), 3.79 (s, 3H). | |
| | yl)phenyl)acrylamide | | |
| 478 | N-(4-(4-amino-7-cyano- | ¹H NMR (400 MHz, DMSO) δ 10.30 (s, 1H), | 505 |
| | 3-(3-fluoro-4-(pyridin- | 8.21 (s, 1H), 8.13 (m, 1H), 7.95-7.83 (m, 1H), | |
| | 2-yloxy)phenyl)-1- | 7.72 (m, 2H), 7.31 (t, J = 18 Hz, 3H), 7.22 (m, | |
| | methyl-1H-pyrrolo[3,2- | 2H), 7.18-7.05 (m, 1H), 6.44 (m, 1H), 6.27 (m, | |
| | c]pyridin-2-yl)phenyl) | 1H), 5.78 (m, 1H), 3.79 (s, 3H). | |
| | acrylamide | | |
| 479 | N-(4-(4-amino-7-cyano- | ¹H NMR (400 MHz, DMSO) δ 10.31 (s, 1H), | 523 |
| | 3-(3-fluoro-4-((5- | 8.20 (d, J = 2.0 Hz, 1H), 8.15 (d, J = 3.1 Hz, | |
| | fluoropyridin-2-yl)oxy) | 1H), 7.86 (dd, J = 9.6, 7.3 Hz, 1H), 7.72 (d, J = | |
| | phenyl)-1-methyl-1H- | 8.5 Hz, 2H), 7.31 (dd, J = 8.4, 6.8 Hz, 4H), 7.21 | |
| | pyrrolo[3,2-c]pyridin-2- | (dd, J = 9.0, 3.6 Hz, 3H), 7.10 (d, J = 9.8 Hz, | |
| | yl)phenyl)acrylamide | 1H), 6.42 (d, J = 10.1 Hz, 1H), 6.30 (d, J = 1.9 | |
| | | Hz, 1H), 5.78 (dd, J = 10.1, 1.9 Hz, 1H), 3.79 (s, | |
| | | 3H). | |
| 480 | N-(4-(4-amino-7-cyano- | ¹H NMR (400 MHz, DMSO) δ 10.30 (s, 1H), | 534 |
| | 3-(4-((4,5-dimethyl- | 8.29 (s, 1H), 8.21 (s, 1H), 7.73 (d, J = 8.4 Hz, | |
| | pyrimidin-2-yl)oxy)-3- | 2H), 7.33 (m, 3H), 7.23 (m, 1H), 7.11 (m, 1H), | |
| | fluorophenyl)-1-methyl- | 6.44 (m, 1H), 6.28 (m, 1H), 5.78 (m, 1H), 3.80 | |
| | 1H-pyrrolo[3,2- | (s, 3H), 2.35 (s, 3H), 2.16 (s, 3H). | |
| | c]pyridin-2-yl)phenyl) | | |
| | acrylamide | | |
| 481 | N-(4-(4-amino-7- | ¹H NMR (400 MHz, DMSO) δ 10.11 (s, 1H), | 524 |
| | cyano-3-(4-(cyclo- | 8.20 (d, J = 14.8 Hz, 2H), 7.74 (d, J = 11.2 Hz, | |
| | hexyloxy)phenyl)-1- | 1H), 7.47 (d, J = 8.4 Hz, 1H), 7.25 (t, J = 16.8 | |
| | methyl-1H-pyrrolo | Hz, 1H), 7.11 (s, 1H), 6.91 (d, J = 8.4 Hz, 2H), | |
| | [3,2-c]pyridin-2-yl)-3- | 5.81 (s, 1H), 5.57 (s, 1H), 4.31 (s, 1H), 3.73 (s, | |
| | fluorophenyl)methacrylamide | 3H), 1.94 (s, 5H), 1.71 (s, 2H), 1.61-1.13 (m, 6H). | |
| 482 | 4-(4-amino-7-cyano-2- | ¹H NMR (400 MHz, DMSO) δ 10.14 (s, 1H), | 607 |
| | (2-fluoro-4- | 8.68 (s, 1H), 8.39 (s, 1H), 7.78-7.75 (m, 1H), | |
| | methacrylamidophenyl)- | 7.60-7.58 (d, J = 8.0 Hz, 1H), 7.53-7.50 (m, 1H), | |
| | 1-methyl-1H- | 7.33-7.29 (t, J = 8.0 Hz, 1H), 6.96 (s, 1H), 6.87- | |
| | pyrrolo[3,2-c]pyridin-3- | 6.85 (d, J = 8.0 Hz, 1H), 5.82 (s, 1H), 5.58 (s, | |
| | yl)-2-methoxy-N-(1- | 1H), 3.80 (s, 3H), 3.74 (s, 3H), 1.94 (s, 3H), | |
| | (trifluoromethyl)cyclo- | 1.31-1.27 (m, 2H), 1.13 (s, 2H). | |
| | propyl)benzamide | | |
| 483 | N-(4-(4-amino-7- | ¹H NMR (400 MHz, DMSO) δ 10.12 (s, 1H), | 519 |
| | cyano-1-methyl-3-(4- | 8.21 (s, 1H), 8.19 (m, 1H), 7.87 (m, 1H), 7.77 | |
| | (pyridin-2-yloxy) | (dd, J = 12.4, 2.0 Hz, 1H), 7.50 (m, 1H), 7.28 | |
| | phenyl)-1H-pyrrolo | (m, 3H), 7.18-7.10 (m, 3H), 7.05 (d, J = 8.4 Hz, | |
| | [3,2-c]pyridin-2-yl)-3- | 1H), 5.81 (s, 1H), 5.58 (s, 1H), 3.75 (s, 3H), 1.94 | |
| | fluorophenyl)methacrylamide | (s, 3H). | |
| 484 | N-(4-(4-amino-7-cyano- | ¹H NMR (400 MHz, DMSO) δ 10.28 (s, 1H), | 487 |
| | 1-methyl-3-(4- | 8.20-8.17 (m, 2H), 7.92-7.81 (m, 1H), 7.70 (d, | |
| | (pyridin-2-yloxy) | J = 8.4 Hz, 2H), 7.29 (m, 4H), 7.20-7.10 (m, | |
| | phenyl)-1H- | 3H), 7.04 (m, 1H), 6.43 (m, 1H), 6.27 (m, 1H), | |
| | pyrrolo[3,2-c]pyridin-2- | 5.78 (m, 1H), 3.80 (s, 3H). | |
| | yl)phenyl)acrylamide | | |
| 485 | N-(4-(4-amino-7-cyano- | ¹H NMR (400 MHz, DMSO) δ 10.17 (s, 1H), | 556 |
| | 3-(3-fluoro-4-((5- | 8.77 (s, 2H), 8.34 (s, 1H), 7.82-7.78 (m, 1H), | |
| | fluoropyrimidin-2-yl) | 7.56-7.54 (m, 1H), 7.44-7.39 (t, J = 8.0 Hz, 1H), | |
| | oxy)phenyl)-1-methyl- | 7.37-7.33 (t, J = 8.0 Hz, 1H), 7.28-7.25 (d, J = | |
| | 1H-pyrrolo[3,2- | 12.0 Hz, 1H), 7.12-7.10 (d, J = 8.0 Hz, 1H), 5.83 | |
| | c]pyridin-2-yl)-3- | (s, 1H), 5.59 (s, 1H), 3.78 (s, 3H), 1.95 (s, 3H). | |
| | fluorophenyl)methacrylamide | | |
| 486 | N-(4-(4-amino-3-(3- | ¹H NMR (400 MHz, DMSO) δ 10.32 (s, 1H), | 554 |
| | chloro-4-((5-fluoro-4- | 8.55 (s, 1H), 8.21 (s, 1H), 7.73 (d, J = 8.4 Hz, | |
| | methylpyrimidin-2- | 2H), 7.44 (s, 1H), 7.31 (m, 4H), 6.44 (m, 1H), | |
| | yl)oxy)phenyl)-7- | 6.27 (d, J = 17.2 Hz, 1H), 5.78 (d, J = 10 Hz, | |
| | cyano-1-methyl-1H- | 1H), 3.79 (s, 3H), 2.41 (s, 3H). | |
| | pyrrolo[3,2-c]pyridin-2- | | |
| | yl)phenyl)acrylamide | | |
| 487 | N-(4-(4-amino-3-(4-((6- | ¹H NMR (400 MHz, DMSO) δ 10.14 (s, 1H), | 571 |
| | chloropyridin-2-yl)oxy)- | 8.23 (s, 1H), 7.99-7.87 (m, 1H), 7.84-7.67 (m, | |
| | 3-fluorophenyl)-7- | 1H), 7.57-7.48 (m, 1H), 7.39-7.18 (m, 4H), | |
| | cyano-1-methyl-1H- | 7.16-7.05 (m, 2H), 5.81 (s, 1H), 5.58 (s, 1H), | |
| | pyrrolo[3,2-c]pyridin-2- | 3.77 (s, 3H), 1.94 (s, 3H). | |
| | yl)-3-fluorophenyl) | | |
| | methacrylamide | | |
| 490 | N-(4-(4-amino-7- | ¹H NMR (400 MHz, DMSO) δ 10.14 (s, 1H), | 570 |
| | cyano-3-(2,5-difluoro- | 8.49 (s, 1H), 8.23 (s, 1H), 7.79 (d, J = 12.8 Hz, | |
| | 4-((4-methylpyrimidin- | 1H), 7.59-7.06 (m, 5H), 6.18 (s, 2H), 5.82 (s, | |
| | 2-yl)oxy)phenyl)-1- | 1H), 5.58 (s, 1H), 3.78 (s, 3H), 2.41 (s, 3H), 1.95 | |
| | methyl-1H-pyrrolo[3,2- | (s, 3H). | |
| | c]pyridin-2-yl)-3- | | |
| | fluorophenyl)methacrylamide | | |
| 491 | N-(4-(4-amino-7- | ¹H NMR (400 MHz, DMSO) δ 10.15 (s, 1H), | 570 |
| | cyano-3-(2,3-difluoro- | 8.50 (d, J = 5.0 Hz, 1H), 8.23 (s, 1H), 7.78 (dd, | |
| | 4-((4-methylpyrimidin- | J = 12.8, 4.4 Hz, 1H), 7.53 (dd, J = 8.4, 1.8 Hz, | |
| | 2-yl)oxy)phenyl)-1- | 1H), 7.30-6.96 (m, 4H), 6.21 (s, 2H), 5.81 (s, | |
| | methyl-1H-pyrrolo[3,2- | 1H), 5.58 (s, 1H), 3.79 (d, J = 4.8 Hz, 3H), 2.41 | |
| | c]pyridin-2-yl)-3- | (s, 3H), 1.95 (s, 3H). | |
| | fluorophenyl)methacrylamide | | |
| 492 | N-(4-(4-amino-7- | ¹H NMR (400 MHz, DMSO) δ 9.60 (s, 1H), 8.46 | 566 |
| | cyano-3-(3-fluoro-4- | (d, J = 5.2 Hz, 1H), 8.36 (s, 1H), 7.57 (t, J = 8.0 | |
| | ((4-methylpyrimidin-2- | Hz, 1H), 7.35 (t, J = 8.4 Hz, 1H), 7.24-7.20 (m, | |
| | yl)oxy)phenyl)-1- | 1H), 7.18 (d, J = 5.2 Hz, 2H), 7.07 (d, J = 8.4 | |
| | methyl-1H-pyrrolo[3,2- | Hz, 1H), 5.87 (s, 1H), 5.56 (s, 1H), 3.71 (s, 3H), | |
| | c]pyridin-2-yl)-2- | 2.39 (s, 3H), 1.96-1.91 (m, 6H). | |
| | fluoro-3-methylphenyl) | | |
| | methacrylamide | | |
| 493 | N-(4-(4-amino-7- | ¹H NMR (400 MHz, DMSO) δ 10.12 (s, 1H), | 552 |
| | cyano-3-(2-fluoro-4- | 8.49 (d, J = 5.0 Hz, 1H), 8.22 (s, 1H), 7.76 (d, | |
| | ((4-methylpyrimidin-2- | J = 11.8 Hz, 1H), 7.50 (d, J = 8.1 Hz, 1H), 7.43 (s, | |
| | yl)oxy)phenyl)-1- | 1H), 7.25-7.18 (m, 3H), 7.12-7.03 (m, 1H), | |
| | methyl-1H-pyrrolo[3,2- | 5.81 (s, 1H), 5.57 (s, 1H), 3.79 (s, 3H), 2.41 (s, | |
| | c]pyridin-2-yl)-3- | 3H), 1.94 (s, 3H). | |
| | fluorophenyl)methacrylamide | | |
| 494 | N-(4-(4-amino-7- | ¹H NMR (400 MHz, DMSO) δ 10.15 (s, 1H), | 555 |
| | cyano-3-(3-fluoro-4- | 8.23 (s, 1H), 8.15 (d, J = 3.1 Hz, 1H), 7.89-7.82 | |
| | ((5-fluoropyridin-2- | (m, 1H), 7.79 (dd, J = 12.4, 1.9 Hz, 1H), 7.52 (d, | |
| | yl)oxy)phenyl)-1- | J = 1.9 Hz, 1H), 7.32 (d, J = 6.0 Hz, 2H), 7.21 | |
| | methyl-1H-pyrrolo | (dd, J = 9.1, 3.5 Hz, 2H), 7.09 (d, J = 7.9 Hz, | |
| | [3,2-c]pyridin-2-yl)-3- | 1H), 5.82 (s, 1H), 5.59 (s, 1H), 3.75 (s, 3H), 1.95 | |
| | fluorophenyl)methacrylamide | (s, 3H). | |
| 495 | N-(4-(4-amino-7- | ¹H NMR (400 MHz, DMSO) δ 10.31 (s, 1H), | 505 |
| | cyano-3-(4-((5- | 8.37 (s, 1H), 8.20 (d, J = 3.2 Hz, 1H), 7.85 (ddd, | |
| | fluoropyridin-2- | J = 9.2, 8.0, 3.2 Hz, 1H), 7.72 (d, J = 8.8 Hz, | |
| | yl)oxy)phenyl)-1- | 2H), 7.38-7.24 (m, 4H), 7.15 (dd, J = 5.6, 3.2 | |
| | methyl-1H-pyrrolo[3,2- | Hz, 3H), 6.44 (dd, J = 17.2, 10.0 Hz, 1H), 6.27 | |
| | c]pyridin-2- | (dd, J = 17.2, 2.0 Hz, 1H), 5.79 (dd, J = 10.0, 2.0 | |
| | yl)phenyl)acrylamide | Hz, 1H), 3.85 (s, 3H). | |
| 496 | N-(4-(4-amino-7- | ¹H NMR (400 MHz, DMSO) δ 10.29 (s, 1H), | 534 |
| | cyano-3-(4-((5-fluoro- | 8.52 (d, J = 1.2 Hz, 1H), 8.19 (s, 1H), 7.72-7.70 | |
| | 4-methylpyrimidin-2- | (d, J = 8.0 Hz, 2H), 7.33-7.31 (d, J = 8.0 Hz, | |
| | yl)oxy)-3-methyl- | 2H), 7.24 (s, 1H), 7.14-7.09 (m, 2H), 6.47-6.40 | |
| | phenyl)-1-methyl-1H- | (m, 1H), 6.29-6.25 (m, 1H), 5.79-5.76 (m, 1H), | |
| | pyrrolo[3,2-c]pyridin-2- | 3.80 (s, 3H), 2.40 (d, J = 2.4 Hz, 3H), 2.05 (s, | |
| | yl)phenyl)acrylamide | 3H). | |
| 497 | N-(4-(4-amino-7- | ¹H NMR (400 MHz, DMSO) δ 10.34 (s, 1H), | 556 |
| | cyano-3-(3,5-difluoro- | 8.59 (s, 1H), 8.35 (s, 1H), 7.76 (d, J = 8.4 Hz, | |
| | 4-((5-fluoro-4-methyl- | 2H), 7.33 (d, J = 8.4 Hz, 2H), 7.14 (d, J = 8.4 | |
| | pyrimidin-2-yl)oxy) | Hz, 2H), 6.45 (dd, J = 16.8, 10.4 Hz, 1H), 6.28 | |
| | phenyl)-1-methyl-1H- | (d, J = 19.2 Hz, 1H), 5.79 (d, J = 11.6 Hz, 1H), | |
| | pyrrolo[3,2-c]pyridin-2- | 3.82 (s, 3H), 2.42 (d, J = 2.4 Hz, 3H). | |
| | yl)phenyl)acrylamide | | |
| 498 | N-(4-(4-amino-7- | ¹H NMR (400 MHz, DMSO) δ 10.51 (s, 1H), | 538 |
| | cyano-3-(4-((5-fluoro- | 8.55 (d, J = 1.2 Hz, 1H), 8.21 (s, 1H), 7.78 (dd, | |
| | 4-methylpyrimidin-2- | J = 12.0, 1.6 Hz, 1H), 7.39 (dd, J = 8.4, 2.0 Hz, | |
| | yl)oxy)phenyl)-1- | 1H), 7.34-7.28 (m, 3H), 7.20 (d, J = 8.8 Hz, | |
| | methyl-1H-pyrrolo[3,2- | 2H), 6.42 (dd, J = 17.2, 10.0 Hz, 1H), 6.30 (dd, | |
| | c]pyridin-2-yl)-3- | J = 17.2, 2.0 Hz, 1H), 5.82 (dd, J = 10.0, 2.0 Hz, | |
| | fluorophenyl)acrylamide | 1H), 3.75 (s, 3H), 2.40 (d, J = 2.4 Hz, 3H). | |
| 500 | N-(4-(4-amino-7- | ¹H NMR (400 MHz, DMSO) δ 10.11 (s, 1H), | 522 |
| | cyano-1-methyl-3-(4- | 8.19 (s, 1H), 7.75 (m, 1H), 7.65 (d, J = 2.3 Hz, | |
| | ((1-methyl-1H-pyrazol- | 1H), 7.48 (m, 1H), 7.25 (t, J = 8.4 Hz, 1H), 7.20 | |
| | 3-yl)oxy)phenyl)-1H- | (d, J = 7.4 Hz, 2H), 7.03 (d, J = 8.8 Hz, 2H), | |
| | pyrrolo[3,2-c]pyridin-2- | 5.89 (d, J = 2.3 Hz, 1H), 5.81 (s, 1H), 5.57 (s, | |
| | yl)-3-fluorophenyl) | 1H), 3.74 (s, 6H), 1.94 (s, 3H). | |
| | methacrylamide | | |
| 502 | N-(4-(4-amino-7- | ¹H NMR (400 MHz, DMSO) δ 10.13 (s, 1H), | 537 |
| | cyano-3-(4-((5-fluoro- | 8.29-8.09 (m, 2H), 7.89-7.80 (m, 1H), 7.79- | |
| | pyridin-2-yl)oxy) | 7.70 (m, 1H), 7.53-7.46 (m, 1H), 7.33-7.18 | |
| | phenyl)-1-methyl-1H- | (m, 3H), 7.17-7.05 (m, 3H), 5.81 (s, 1H), 5.58 | |
| | pyrrolo[3,2-c]pyridin-2- | (s, 1H), 3.75 (s, 3H), 1.94 (s, 3H). | |
| | yl)-3-fluorophenyl) | | |
| | methacrylamide | | |
| 503 | N-(4-(4-amino-7- | ¹H NMR (400 MHz, DMSO) δ 10.31 (s, 1H), | 556 |
| | cyano-3-(2,3-difluoro- | 8.59 (d, J = 1.1 Hz, 1H), 8.21 (s, 1H), 7.73 (d, | |
| | 4-((5-fluoro-4-methyl- | J = 8.6 Hz, 2H), 7.28 (d, J = 8.6 Hz, 2H), 7.22 (dd, | |
| | pyrimidin-2-yl)oxy) | J = 15.2, 7.6 Hz, 2H), 6.48-6.40 (m, 1H), 6.28 | |
| | phenyl)-1-methyl-1H- | (dd, J = 17.0, 1.9 Hz, 1H), 6.11 (s, 2H), 5.79 (dd, | |
| | pyrrolo[3,2-c]pyridin-2- | J = 10.1, 1.9 Hz, 1H), 3.84 (s, 3H), 2.42 (s, 3H). | |
| | yl)phenyl)acrylamide | | |
| 504 | N-(4-(4-amino-7-cyano- | ¹H NMR (400 MHz, DMSO) δ 10.22 (s, 1H), | 534 |
| | 3-(4-((5-fluoro-4- | 8.54 (d, J = 1.3 Hz, 1H), 8.19 (s, 1H), 7.57 (dd, | |
| | methylpyrimidin-2-yl) | J = 15.2, 6.6 Hz, 2H), 7.32-7.11 (m, 5H), 6.43 | |
| | oxy)phenyl)-1-methyl- | (m, 1H), 6.26 (m, 1H), 5.77 (m, 1H), 3.64 (s, | |
| | 1H-pyrrolo[3,2-c] | 3H), 2.39 (d, J = 2.5 Hz, 3H), 1.99 (s, 3H). | |
| | pyridin-2-yl)-3-methyl- | | |
| | phenyl)acrylamide | | |
| 505 | N-(4-(4-amino-7- | ¹H NMR (400 MHz, DMSO) δ 10.47 (s, 1H), | 554 |
| | cyano-3-(4-((5-fluoro- | 8.54 (d, J = 1.3 Hz, 1H), 8.22 (s, 1H), 8.04 (d, | |
| | 4-methylpyrimidin-2- | J = 2.0 Hz, 1H), 7.56 (dd, J = 8.4, 2.0 Hz, 1H), | |
| | yl)oxy)phenyl)-1- | 7.39 (d, J = 8.4 Hz, 1H), 7.29 (d, J = 7.4 Hz, | |
| | methyl-1H-pyrrolo[3,2- | 2H), 7.19 (d, J = 8.8 Hz, 2H), 6.42 (dd, J = 17.0, | |
| | c]pyridin-2-yl)-3-chloro- | 10.0 Hz, 1H), 6.30 (dd, J = 17.0, 2.0 Hz, 1H), | |
| | phenyl)acrylamide | 5.82 (dd, J = 10.0, 2.0 Hz, 1H), 3.69 (s, 3H), | |
| | | 2.39 (d, J = 2.5 Hz, 3H). | |
| 506 | N-(4-(4-amino-7- | ¹H NMR (400 MHz, DMSO) δ 10.28 (s, 1H), | 492 |
| | cyano-3-(4- | 8.35 (s, 1H), 7.69 (d, J = 8.0 Hz, 2H), 7.27 (d, | |
| | (cyclohexyloxy)phenyl)- | J = 8.4 Hz, 2H), 7.14 (d, J = 8.4 Hz, 2H), 6.93 (d, | |
| | 1-methyl-1H- | J = 8.0 Hz, 2H), 6.43 (dd, J = 16.8, 10.4 Hz, 1H), | |
| | pyrrolo[3,2-c]pyridin-2- | 6.26 (d, J = 15.2 Hz, 1H), 5.78 (d, J = 11.6 Hz, | |
| | yl)phenyl)acrylamide | 1H), 4.32 (m, 1H), 3.83 (s, 3H), 1.91 (s, 2H), | |
| | | 1.71 (s, 2H), 1.52 (s, 1H), 1.38 (d, J = 9.6 Hz, | |
| | | 4H), 1.23 (s, 1H). | |
| 507 | N-(4-(4-amino-3-(4-((5- | ¹H NMR (400 MHz, DMSO) δ 10.34 (s, 1H), | 554 |
| | chloro-4- | 8.63 (s, 1H), 8.42 (s, 1H), 7.76-7.74 (d, J = 8.0 | |
| | methylpyrimidin-2-yl) | Hz, 2H), 7.42-7.38 (t, J = 8.0 Hz, 1H), 7.33-7.31 | |
| | oxy)-3-fluorophenyl)-7- | (d, J = 8.0 Hz, 2H), 7.30-7.27 (m, 1H), 7.13-7.11 | |
| | cyano-1-methyl-1H- | (m, 1H), 6.48-6.41 (m, 1H), 6.30-6.26 (m, 1H), | |
| | pyrrolo[3,2-c]pyridin-2- | 5.81-5.78 (m, 1H), 3.85 (s, 3H), 2.47 (s, 3H). | |
| | yl)phenyl)acrylamide | | |
| 508 | N-(4-(4-amino-7- | ¹H NMR (400 MHz, DMSO) δ 10.30 (s, 1H), | 522 |
| | cyano-3-(4-(cyclo- | 8.22 (s, 1H), 7.70 (m, 3H), 7.27 (d, J = 8.4 Hz, | |
| | hexanecarbonyl)-3- | 2H), 7.15-7.09 (m, 2H), 6.43 (m, 1H), 6.27 (m, | |
| | fluorophenyl)-1-methyl- | 1H), 5.78 (m, 1H), 3.79 (s, 3H), 3.09 (s, 1H), | |
| | 1H-pyrrolo[3,2- | 1.83 (m, 4H), 1.72 (m, 2H), 1.27 (m, 4H). | |
| | c]pyridine-2- | | |
| | yl)phenyl)acrylamide | | |
| 509 | N-(4-(4-amino-7- | ¹H NMR (400 MHz, DMSO) δ 10.30 (s, 1H), | 511 |
| | cyano-3-(3-fluoro-4- | 8.21 (s, 1H), 7.78-7.65 (m, 2H), 7.54-7.46 (m, | |
| | (thiazol-2-yloxy) | 1H), 7.33-7.27 (m, 3H), 7.27-7.22 (m, 2H), | |
| | phenyl)-1-methyl-1H- | 7.16-7.07 (m, 1H), 6.49-6.37 (m, 1H), 6.31-6.22 (m, 1H), | |
| | pyrrolo[3,2-c]pyridin-2- | 6.07 (s, 2H), 5.83-5.72 (m, 1H), 3.80 (s, 3H). | |
| | yl)phenyl)acrylamide | | |
| 510 | N-(4-(4-amino-7- | ¹H NMR (400 MHz, DMSO) δ 10.32 (s, 1H), | 525 |
| | cyano-3-(3-fluoro-4- | 8.35 (s, 1H), 7.73 (d, J = 8.4 Hz, 2H), 7.51 (t, J = | |
| | ((4-methylthiazol-2- | 8.4 Hz, 1H), 7.31 (dd, J = 13.2, 5.2 Hz, 3H), 7.11 | |
| | yl)oxy)phenyl)-1- | (dd, J = 8.4, 1.0 Hz, 1H), 6.81 (d, J = 1.2 Hz, | |
| | methyl-1H-pyrrolo[3,2- | 1H), 6.44 (dd, J = 17.2, 10.0 Hz, 1H), 6.27 (dd, | |
| | c]pyridin-2-yl) | J = 17.2, 2.0 Hz, 1H), 5.78 (dd, J = 10.0, 2.0 Hz, | |
| | phenyl)acrylamide | 1H), 3.84 (s, 3H), 2.18 (d, J = 1.2 Hz, 3H). | |
| 511 | N-(4-(4-amino-7-cyano- | ¹H NMR (400 MHz, DMSO) δ 10.33 (s, 1H), | 574 |
| | 3-(3-fluoro-4-((4- | 9.04 (d, J = 4.8 Hz, 1H), 8.39 (s, 1H), 7.85 (d, | |
| | (trifluoromethyl)pyr- | J = 4.8 Hz, 1H), 7.74 (d, J = 8.4 Hz, 2H), 7.48 (t, | |
| | imidin-2-yl)oxy)phenyl)-1- | J = 8.4 Hz, 1H), 7.32 (d, J = 8.8 Hz, 3H), 7.15 (d, | |
| | methyl-1H-pyrrolo[3,2- | J = 8.4 Hz, 1H), 6.44 (dd, J = 17.2, 10.1 Hz, 1H), | |
| | c]pyridin-2-yl)phenyl) | 6.28 (dd, J = 16.8, 1.6 Hz, 1H), 5.79 (dd, J = | |
| | acrylamide | 10.0, 1.6 Hz, 1H), 3.85 (s, 3H). | |
| 512 | N-(4-(4-amino-7- | ¹H NMR (400 MHz, DMSO) δ 10.14 (s, 1H), | 606 |
| | cyano-3-(3-fluoro-4- | 9.04 (d, J = 4.8 Hz, 1H), 8.23 (s, 1H), 7.84 (d, | |
| | ((4-(trifluoromethyl) | J = 5.2 Hz, 1H), 7.78 (dd, J = 12.4, 2.0 Hz, 1H), | |
| | pyrimidin-2-yl)oxy) | 7.53 (dd, J = 8.4, 2.0 Hz, 1H), 7.47 (t, J = 8.4 Hz, | |
| | phenyl)-1-methyl-1H- | 1H), 7.33 (t, J = 8.4 Hz, 1H), 7.28 (d, J = 11.2 | |
| | pyrrolo[3,2-c]pyridin-2- | Hz, 1H), 7.14 (d, J = 8.0 Hz, 1H), 5.81 (s, 1H), | |
| | yl)-3-fluorophenyl) | 5.58 (s, 1H), 3.76 (s, 3H), 1.95 (s, 3H). | |
| | methacrylamide | | |
| 514 | N-(4-(4-amino-7- | ¹H NMR (400 MHz, DMSO) δ 9.90 (s, 1H), 8.47 | 570 |
| | cyano-3-(3-fluoro-4- | (d, J = 4.8 Hz, 1H), 8.25 (s, 1H), 7.47 (t, J = 7.2 | |
| | ((4-methylpyrimidin-2- | Hz, 1H), 7.38 (t, J = 8.4 Hz, 1H), 7.28-7.16 (m, | |
| | yl)oxy)phenyl)-1- | 3H), 7.12 (d, J = 8.0 Hz, 1H), 6.17 (br, 2H), 5.89 | |
| | methyl-1H-pyrrolo[3,2- | (s, 1H), 5.60 (s, 1H), 3.80 (s, 3H), 2.40 (s, 3H), | |
| | c]pyridin-2-yl)-2,3- | 1.95 (s, 3H). | |
| | difluorophenyl)methacrylamide | | |
| 515 | N-(4-(4-amino-7- | ¹H NMR (400 MHz, DMSO) δ 10.16 (s, 1H), | 569 |
| | cyano-3-(3-fluoro-4- | 8.39 (s, 1H), 7.83-7.66 (m, 2H), 7.55 (d, J = 8.4 | |
| | ((5-fluoro-6-methyl- | Hz, 1H), 7.29 (dt, J = 11.6, 8.4 Hz, 2H), 7.22 (d, | |
| | pyridin-2-yl)oxy) | J = 11.2 Hz, 1H), 7.08-6.91 (m, 2H), 5.81 (s, | |
| | phenyl)-1-methyl-1H- | 1H), 5.59 (s, 1H), 3.81 (s, 3H), 2.24 (d, J = 3.2 | |
| | pyrrolo[3,2-c]pyridin-2- | Hz, 3H), 1.95 (s, 3H). | |
| | yl)-3-fluorophenyl) | | |
| | methacrylamide | | |
| 516 | N-(4-(4-amino-7- | ¹H NMR (400 MHz, DMSO) δ 10.32 (s, 1H), | 537 |
| | cyano-3-(3-fluoro-4- | 8.39 (s, 1H), 7.73 (dd, J = 8.0, 3.2 Hz, 3H), 7.28 | |
| | ((5-fluoro-6-methyl- | (dd, J = 13.2, 8.4 Hz, 4H), 7.07 (d, J = 8.0 Hz, | |
| | pyridin-2-yl)oxy) | 1H), 6.97 (dd, J = 8.8, 2.8 Hz, 1H), 6.44 (dd, J = | |
| | phenyl)-1-methyl-1H- | 17.2, 10.0 Hz, 1H), 6.28 (dd, J = 17.2, 2.0 Hz, | |
| | pyrrolo[3,2-c]pyridin-2- | 1H), 5.79 (dd, J = 10.0, 2.0 Hz, 1H), 3.87 (s, | |
| | yl)phenyl)acrylamide | 3H), 2.25 (d, J = 2.8 Hz, 3H). | |
| 517 | N-(4-(4-amino-7- | ¹H NMR (400 MHz, DMSO) δ 10.28 (s, 1H), | 556 |
| | cyano-3-(4-((5-fluoro- | 8.55 (s, 1H), 8.23 (s, 1H), 7.94 (t, J = 7.6 Hz, | |
| | 4-methylpyrimidin-2- | 1H), 7.31 (d, J = 8.3 Hz, 2H), 7.21 (t, J = 8.9 Hz, | |
| | yl)oxy)phenyl)-1- | 3H), 6.64 (dd, J = 17.0, 10.2 Hz, 1H), 6.31 (dd, | |
| | methyl-1H-pyrrolo[3,2- | J = 17.0, 1.7 Hz, 1H), 5.87-5.78 (m, 1H), 3.79 (s, | |
| | c]pyridin-2-yl)-2,3- | 3H), 2.40 (d, J = 2.4 Hz, 3H). | |
| | difluorophenyl)acrylamide | | |
| 518 | N-(4-(4-amino-7- | ¹H NMR (400 MHz, DMSO) δ 10.11 (s, 1H), | 542 |
| | cyano-3-(4- | 8.19 (s, 1H), 7.75 (dd, J = 12.4, 1.5 Hz, 1H), | |
| | (cyclohexyloxy)-3- | 7.49 (dd, J = 8.4, 1.6 Hz, 1H), 7.33-6.85 (m, | |
| | fluorophenyl)-1-methyl- | 4H), 6.05 (s, 1H), 5.81(s, 1H), 5.58 (s, 1H), 4.46- | |
| | 1H-pyrrolo[3,2-c] | 4.26 (m, 1H), 3.73 (s, 3H), 1.93 (d, J = 14.8 Hz, | |
| | pyridin-2-yl)-3- | 5H), 1.70 (d, J = 5.2 Hz, 2H), 1.56-0.93 (m, 6). | |
| | fluorophenyl)methacrylamide | | |
| 519 | N-(4-(4-amino-7- | ¹H NMR (400 MHz, DMSO) δ 10.28 (s, 1H), | 522 |
| | cyano-3-(4-((1,5- | 8.19 (s, 1H), 7.71-7.69 (d, J = 8.0 Hz, 2H), 7.29- | |
| | dimethyl-1H-pyrazol-3- | 7.26 (d, J = 12.0 Hz, 2H), 7.19-7.13 (m, 2H), | |
| | yl)oxy)-3-fluorophenyl)- | 7.01-6.99 (m, 1H), 6.47-6.40 (m, 1H), 6.29-6.25 | |
| | 1-methyl-1H-pyrrolo | (m, 1H), 5.79-5.76 (m, 1H), 5.67 (s, 1H), 3.79 (s, | |
| | [3,2-c]pyridin-2- | 3H), 3.59 (s, 3H), 2.20 (s, 3H). | |
| | yl)phenyl)acrylamide | | |
| 520 | N-(4-(4-amino-7-cyano- | ¹H NMR (400 MHz, DMSO) δ 10.29 (s, 1H), | 576 |
| | 3-(3-fluoro-4-((1- | 8.20 (s, 1H), 7.71 (d, J = 8.4 Hz, 2H), 7.30-7.19 | |
| | methyl-5- | (m, 4H), 7.03 (d, J = 8.4 Hz, 1H), 6.65 (s, 1H), | |
| | (trifluoromethyl)-1H- | 6.43 (dd, J = 17.2, 10.0 Hz, 1H), 6.27 (dd, J = | |
| | pyrazol-3-yl)oxy) | 17.2, 2.0 Hz, 1H), 5.78 (dd, J = 10.0, 2.0 Hz, | |
| | phenyl)-1-methyl-1H- | 1H), 3.85 (s, 3H), 3.79 (s, 3H). | |
| | pyrrolo[3,2-c]pyridin-2- | | |
| | yl)phenyl)acrylamide | | |
| 521 | N-(4-(4-amino-7- | ¹H NMR (400 MHz, DMSO) δ 10.26 (s, 1H), | 558 |
| | cyano-1-methyl-3-(4- | 8.18 (s, 1H), 7.68 (d, J = 8.4 Hz, 2H), 7.26 (t, J = | |
| | ((1-methyl-5- | 8.4 Hz, 4H), 7.09 (d, J = 8.4 Hz, 2H), 6.67 (s, | |
| | (trifluoromethyl)-1H- | 1H), 6.43 (dd, J = 17.2, 10.0 Hz, 1H), 6.26 (dd, | |
| | pyrazol-3-yl)oxy) | J = 17.2, 2.0 Hz, 1H), 5.77 (dd, J = 10.0, 2.0 Hz, | |
| | phenyl)-1H-pyrrolo | 1H), 3.87 (s, 3H), 3.80 (s, 3H). | |
| | [3,2-c]pyridin-2-yl) | | |
| | phenyl)acrylamide | | |
| 522 | N-(4-(4-amino-7- | ¹H NMR (400 MHz, DMSO) δ 10.28 (s, 1H), | 556 |
| | cyano-1-methyl-3-(4- | 9.00 (d, J = 4.8 Hz, 1H), 8.20 (s, 1H), 7.79 (d, | |
| | ((4-(trifluoromethyl) | J = 4.9 Hz, 1H), 7.70 (d, J = 8.4 Hz, 2H), 7.36- | |
| | pyrimidin-2-yl)oxy) | 7.29 (m, 6H), 6.49-6.38 (m, 1H), 6.27 (d, J = | |
| | phenyl)-1H-pyrrolo | 15.9 Hz, 1H), 5.78 (d, J = 11.2 Hz, 1H), 3.81 (s, | |
| | [3,2-c]pyridin-2-yl) | 3H). | |
| | phenyl)acrylamide | | |
| 523 | N-(4-(4-amino-7-cyano- | ¹H NMR (400 MHz, DMSO) δ 10.13 (s, 1H), | 588 |
| | 1-methyl-3-(4-((4- | 9.00 (d, J = 4.9 Hz, 1H), 8.22 (s, 1H), 7.77 (m, | |
| | (trifluoromethyl) | 2H), 7.51 (m, 1H), 7.30 (m, 5H), 5.81 (s, 1H), | |
| | pyrimidin-2-yl)oxy) | 5.58 (s, 1H), 3.76 (s, 3H), 1.94 (s, 3H). | |
| | phenyl)-1H-pyrrolo | | |
| | [3,2-c]pyridin-2-yl)-3- | | |
| | fluorophenyl)methacrylamide | | |
| 524 | N-(4-(4-amino-7- | ¹H NMR (400 MHz, DMSO) δ 10.26 (s, 1H), | 516 |
| | cyano-3-(4-((4,6- | 8.19 (s, 1H), 7.70 (d, J = 8.4 Hz, 2H), 7.35-7.24 | |
| | dimethylpyrimidin-2- | (m, 4H), 7.20-7.12 (m, 2H), 7.01 (s, 1H), 6.43 | |
| | yl)oxy)phenyl)-1- | (dd, J = 16.8, 10.0 Hz, 1H), 6.26 (dd, J = 16.8, | |
| | methyl-1H-pyrrolo[3,2- | 2.0 Hz, 1H), 5.77 (dd, J = 10.0, 2.0 Hz, 1H), 3.82 | |
| | c]pyridin-2-yl)phenyl) | (s, 3H), 2.32 (s, 6H). | |
| | acrylamide | | |
| 525 | N-(4-(4-amino-7-cyano- | ¹H NMR (400 MHz, DMSO) δ 10.12 (s, 1H), | 570 |
| | 3-(4-((4- | 8.86 (d, J = 4.8 Hz, 1H), 8.21 (s, 1H), 7.77 (dd, | |
| | (difluoromethyl)pyrimidin- | J = 14.4,1.8 Hz, 1H), 7.61-7.43 (m, 2H), 7.30 | |
| | 2-yl)oxy)phenyl)-1- | (m , 5H), 6.91 (t, J = 108.4 Hz, 1H), 5.81 (s, 1H), | |
| | methyl-1H-pyrrolo[3,2- | 5.58 (s, 1H), 3.76 (s, 3H), 1.94 (s, 3H). | |
| | c]pyridin-2-yl)-3- | | |
| | fluorophenyl)methacrylamide | | |
| 526 | N-(4-(4-amino-7- | ¹H NMR (400 MHz, DMSO) δ 10.08 (s, 1H), | 568 |
| | cyano-3-(4-((5-fluoro- | 8.54 (d, J = 1.0 Hz, 1H), 8.21 (s, 1H), 8.02 (d, | |
| | 4-methylpyrimidin-2- | J = 1.9 Hz, 1H), 7.67 (m, 1H), 7.37 (d, J = 8.4 Hz, | |
| | yl)oxy)phenyl)-1- | 1H), 7.29 (d, J = 7.6 Hz, 2H), 7.19 (d, J = 8.8 | |
| | methyl-1H-pyrrolo[3,2- | Hz, 2H), 5.82 (s, 1H), 5.58 (s, 1H), 3.69 (s, 3H), | |
| | c]pyridin-2-yl)-3- | 2.40 (d, J = 2.4 Hz, 3H), 1.94 (s, 3H). | |
| | chlorophenyl)methacrylamide | | |
| 527 | N-(4-(4-amino-7- | ¹H NMR (400 MHz, DMSO) δ 10.27 (s, 1H), | 490 |
| | cyano-1-methyl-3-(4- | 8.18 (s, 1H), 7.68 (d, J = 8.0 Hz, 2H), 7.36 (d, | |
| | ((1-methyl-1H-pyrazol- | J = 2.0 Hz, 1H), 7.29-7.25 (m, 4H), 7.11 (d, J = | |
| | 5-yl)oxy)phenyl)-1H- | 8.4 Hz, 2H), 6.43 (m, 1H), 6.27 (m, 1H), 5.78 | |
| | pyrrolo[3,2-c]pyridin-2- | (m, 2H), 3.80 (s, 3H), 3.64 (s, 3H). | |
| | yl)phenyl)acrylamide | | |
| 528 | N-(4-(4-amino-7- | ¹H NMR (400 MHz, DMSO) δ 10.09 (s, 1H), | 564 |
| | cyano-1-methyl-3-(3- | 8.43-8.42 (d, J = 4.0 Hz, 1H), 8.21 (s, 1H), 8.03 | |
| | methyl-4-((4-methyl- | (m, 1H), 7.70-7.67 (m, 1H), 7.41-7.39 (d, J = 8.0 | |
| | pyrimidin-2-yl)oxy) | Hz, 1H), 7.22 (s, 1H), 7.13-7.10 (m, 3H), 5.82 | |
| | phenyl)-1H-pyrrolo | (s, 1H), 5.58 (s, 1H), 3.68 (s, 3H), 2.39 (s, 3H), | |
| | [3,2-c]pyridin-2-yl)-3- | 2.03 (s, 3H), 1.94 (s, 3H). | |
| | chlorophenyl)methacrylamide | | |
| 529 | N-(4-(4-amino-7- | ¹H NMR (400 MHz, DMSO) δ 10.28 (s, 1H), | 534 |
| | cyano-3-(4-((1-cyclo- | 8.19 (s, 1H), 7.85-7.62 (m, 3H), 7.28 (d, J = 8.4 | |
| | propyl-1H-pyrazol-3- | Hz, 2H), 7.22-7.13 (m, 2H), 7.01 (d, J = 8.4 Hz, | |
| | yl)oxy)-3-fluoro- | 1H), 6.43 (m, 1H), 6.27 (m, 1H), 5.85 (d, J = 2.4 | |
| | phenyl)-1-methyl-1H- | Hz, 1H), 5.78 (m, 1H), 3.79 (s, 3H), 3.60 (m, | |
| | pyrrolo[3,2-c]pyridin-2- | 1H), 1.01-0.94 (m, 2H), 0.94-0.86 (m, 2H). | |
| | yl)phenyl)acrylamide | | |
| 530 | N-(4-(4-amino-3-(3- | ¹H NMR (400 MHz, DMSO) δ 10.14 (s, 1H), | 584 |
| | chloro-4-((4-methyl- | 8.45 (d, J = 4.8 Hz, 1H), 8.38 (s, 1H), 8.06 (d, | |
| | pyrimidin-2-yl)oxy) | J = 2.0 Hz, 1H), 7.73 (m, 1H), 7.42 (d, J = 8.4 Hz, | |
| | phenyl)-7-cyano-1- | 2H), 7.36 (d, J = 8.4 Hz, 1H), 7.23 (d, J = 7.6 | |
| | methyl-1H-pyrrolo[3,2- | Hz, 1H), 7.17 (d, J = 5.2 Hz, 1H), 5.83 (s, 1H), | |
| | c]pyridin-2-yl)-3- | 5.59 (s, 1H), 3.73 (s, 3H), 2.40 (s, 3H), 1.95 (s, | |
| | chlorophenyl)methacrylamide | 3H). | |
| 531 | N-(4-(4-amino-7- | ¹H NMR (400 MHz, DMSO) δ 10.10 (s, 1H), | 549 |
| | cyano-1-methyl-3-(4- | 8.45-8.27 (m, 1H), 8.03 (s, 1H), 7.76-7.64 (m, | |
| | ((6-methylpyridin-2- | 2H), 7.34 (d, J = 8.4 Hz, 1H), 7.32-7.16 (m, | |
| | yl)oxy)phenyl)-1H- | 2H), 7.10 (d, J = 8.4 Hz, 2H), 7.02 (d, J = 7.6 | |
| | pyrrolo[3,2-c]pyridin-2- | Hz, 1H), 6.78 (d, J = 8.0 Hz, 1H), 5.82 (s, 1H), | |
| | yl)-3-chlorophenyl) | 5.58 (s, 1H), 3.74 (s, 3H), 2.32 (s, 3H), 1.94 (s, | |
| | methacrylamide | 3H). | |
| 532 | 4-(3-acrylamido-12- | ¹H NMR (400 MHz, DMSO) δ 10.32 (s, 1H), | 585 |
| | amino-9-cyano-6,7- | 8.57 (d, J = 6.8 Hz, 1H), 8.22 (s, 1H), 7.69 (m, | |
| | dihydrobenzo[f]pyr- | 2H), 7.24-7.22 (m, 1H), 7.07 (s, 1H), 6.97 (d, J = | |
| | ido[3′,4′:4,5]pyrrolo[1,2- | 8.0 Hz, 1H), 6.86 (d, J = 8.4 Hz, 1H), 6.44-6.38 | |
| | d][1,4]oxazepin-13-yl)- | (m, 1H), 6.30-6.24 (m, 1H), 5.80-5.77 (m, 1H), | |
| | N-(3,3- | 4.59 (s, 4H), 4.30-4.26 (m, 1H), 3.79 (s, 3H), | |
| | difluorocyclobutyl)-2- | 3.00-2.89 (m, 2H), 2.82-2.70 (m, 2H). | |
| | methoxybenzamide | | |
| 533 | N-(4-(4-amino-3-(3- | ¹H NMR (400 MHz, DMSO) δ 9.86 (s, 1H), 8.45 | 564 |
| | chloro-4-((4- | (d, J = 5.2 Hz, 1H), 8.21 (s, 1H), 7.66 (s, 1H), | |
| | methylpyrimidin-2- | 7.61 (d, J = 10 Hz, 1H), 7.39 (d, J = 1.6 Hz, 1H), | |
| | yl)oxy)phenyl)-7- | 7.30 (dd, J = 19.6, 8.3 Hz, 2H), 7.23 (dd, J = 8.4, | |
| | cyano-1-methyl-1H- | 1.6 Hz, 1H), 7.16 (d, J = 5.5 Hz, 1H), 6.08 (s, | |
| | pyrrolo[3,2-c]pyridin-2- | 2H), 5.80 (s, 1H), 5.53 (s, 1H), 3.64 (s, 3H), 2.40 | |
| | yl)-3-methylphenyl) | (s, 3H), 1.96 (d, J = 14.8 Hz, 6H). | |
| | methacrylamide | | |
| 534 | N-(4-(4-amino-7- | ¹H NMR (400 MHz, DMSO-d6) δ 10.09 (s, 1H), | 586 |
| | cyano-3-(4-((4- | 8.86 (d, J = 4.9 Hz, 1H), 8.22 (s, 1H), 8.03 (d, | |
| | (difluoromethyl)pyr- | J = 1.9 Hz, 1H), 7.68 (dd, J = 8.4, 2.0 Hz, 1H), | |
| | imidin-2-yl)oxy)phenyl)-1- | 7.55 (dd, J = 8.8, 5.1 Hz, 2H), 7.38 (d, J = 8.5 | |
| | methyl-1H-pyrrolo[3,2- | Hz, 1H), 7.36-7.23 (m, 2H), 6.91 (t, J = 54.0 | |
| | c]pyridin-2-yl)-3- | Hz, 1H), 5.82 (s, 1H), 5.58 (s, 1H), 3.69 (s, 3H), | |
| | chlorophenyl)methacrylamide | 1.94 (s, 3H). | |
| 535 | N-(4-(4-amino-7- | ¹H NMR (400 MHz, DMSO-d6) δ 10.12 (s, 1H), | 566 |
| | cyano-3-(3-fluoro-4- | 8.42 (s, 1H), 8.05 (s, 1H), 7.73 (t, J = 7.8 Hz, | |
| | ((6-methylpyridin-2- | 1H), 7.37 (d, J = 8.4 Hz, 1H), 7.27 (t, J = 8.4 Hz, | |
| | yl)oxy)phenyl)-1- | 1H), 7.20 (s, 1H), 7.02 (dd, J = 16.0, 8.0 Hz, | |
| | methyl-1H-pyrrolo[3,2- | 2H), 6.85 (d, J = 8.2 Hz, 1H), 5.82 (s, 1H), 5.59 | |
| | c]pyridin-2-yl)-3- | (s, 1H), 3.76 (s, 3H), 2.25 (s, 3H), 1.94 (s, 3H). | |
| | chlorophenyl)methacrylamide | | |
| 536 | N-(4-(4-amino-7-cyano- | ¹H NMR (400 MHz, DMSO-d6) δ 10.51 (s, 1H), | 556 |
| | 3-(4-((4-(difluoro- | 8.86 (d, J = 4.9 Hz, 1H), 8.21 (s, 1H), 7.79 (d, | |
| | methyl)pyrimidin-2-yl) | J = 12.1 Hz, 1H), 7.55 (s, 1H), 7.43-7.23 (m, | |
| | oxy)phenyl)-1-methyl- | 6H), 6.91 (t, J = 54.1 Hz, 1H), 6.42 (dd, J = 16.9, | |
| | 1H-pyrrolo[3,2-c] | 9.9 Hz, 1H), 6.30 (dd, J = 16.9, 1.8 Hz, 1H), 5.82 | |
| | pyridin-2-yl)-3-fluoro- | (dd, J = 10.0, 1.8 Hz, 1H), 3.76 (s, 3H). | |
| | phenyl)acrylamide | | |
| 537 | N-(4-(4-amino-7- | ¹H NMR (400 MHz, DMSO-d6) δ 10.50 (s, 1H), | 520 |
| | cyano-1-methyl-3-(4- | 8.45 (d, J = 5.0 Hz, 1H), 8.21 (s, 1H), 7.79 (dd, | |
| | ((4-methylpyrimidin-2- | J = 12.2, 1.7 Hz, 1H), 7.39 (dd, J = 8.4, 1.8 Hz, | |
| | yl)oxy)phenyl)-1H- | 1H), 7.31 (dd, J = 15.8, 7.6 Hz, 3H), 7.20 (d, J = | |
| | pyrrolo[3,2-c]pyridin-2- | 8.7 Hz, 2H), 7.14 (d, J = 5.0 Hz, 1H), 6.42 (dd, | |
| | yl)-3-fluorophenyl) | J = 17.0, 10.0 Hz, 1H), 6.29 (dd, J = 17.0, 1.9 Hz, | |
| | acrylamide | 1H), 5.82 (dd, J = 10.0, 1.9 Hz, 1H), 3.76 (s, | |
| | | 3H), 2.40 (s, 3H). | |
| 538 | N-(4-(4-amino-7- | ¹H NMR (400 MHz, DMSO-d6) δ 10.08 (s, 1H), | 540 |
| | cyano-3-(4- | 8.32 (s, 1H), 8.01 (s, 1H), 7.65 (d, J = 8.7 Hz, | |
| | (cyclohexyloxy)phenyl)- | 1H), 7.31 (d, J = 8.4 Hz, 1H), 7.11 (m, 2H), 6.91 | |
| | 1-methyl-1H- | (d, J = 8.6 Hz, 2H), 5.82 (s, 1H), 5.58 (s, 1H), | |
| | pyrrolo[3,2-c]pyridin-2- | 4.30 (1, 1H), 3.71 (s, 3H), 1.94 (s, 3H), 1.91 (s, | |
| | yl)-3-chlorophenyl) | 2H), 1.70 (s, 2H), 1.51 (s, 1H), 1.45-1.28 (m, | |
| | methacrylamide | 4H), 1.24 (s, 1H). | |
| 539 | N-(4-(4-amino-5-(4- | ¹H NMR (400 MHz, DMSO-d6) δ 10.36 (s, 1H), | 491 |
| | (cyclohexyloxy)-3- | 8.13 (s, 1H), 7.68 (d, J = 8.7 Hz, 2H), 7.38 (d, | |
| | fluorophenyl)pyrazolo[5,1- | J = 8.7 Hz, 2H), 7.34-7.23 (m, 2H), 7.16-7.06 | |
| | f][1,2,4]triazin-6- | (m, 1H), 5.71 (dd, J = 47.6, 3.7 Hz, 1H), 5.44 | |
| | yl)phenyl)-2- | (dd, J = 15.6, 3.6 Hz, 1H), 4.44 (td, J = 8.8, 4.4 | |
| | fluoroacrylamide | Hz, 1H), 3.83 (s, 3H), 1.99 (dd, J = 9.6, 3.8 Hz, | |
| | | 2H), 1.75 (dd, J = 8.0, 4.1 Hz, 2H), 1.61-1.15 | |
| | | (m, 6H). | |
| 540 | N-(4-(4-amino-5-(4- | ¹H NMR (400 MHz, DMSO-d6) δ 10.22 (s, 1H), | 473 |
| | (cyclohexyloxy)-3- | 8.13 (s, 1H), 7.62 (d, J = 8.7 Hz, 2H), 7.37 (d, | |
| | fluorophenyl)pyrazolo[5,1- | J = 8.7 Hz, 2H), 7.31 (d, J = 8.7 Hz, 1H), 7.26 (dd, | |
| | f][1,2,4]triazin-6- | J = 12.0, 2.2 Hz, 1H), 7.15-7.08 (m, 1H), 6.43 | |
| | yl)phenyl)acrylamide | (dd, J = 17.0, 10.1 Hz, 1H), 6.25 (dd, J = 17.0, | |
| | | 2.0 Hz, 1H), 5.76 (dd, J = 10.1, 2.0 Hz, 1H), 4.45 | |
| | | (dt, J = 8.9, 4.9 Hz, 1H), 3.83 (s, 3H), 1.99 (dd, | |
| | | J = 8.3, 3.9 Hz, 2H), 1.75 (dd, J = 9.2, 3.6 Hz, | |
| | | 2H), 1.69-1.07 (m, 6H). | |
| 541 | N-(4-(4-amino-7- | ¹H NMR (400 MHz, DMSO-d6) δ 10.30 (s, 1H), | 510 |
| | cyano-3-(4- | 8.35 (s, 1H), 7.71 (d, J = 8.6 Hz, 2H), 7.27 (d, | |
| | (cyclohexyloxy)-3- | J = 8.6 Hz, 2H), 7.18 (t, J = 8.8 Hz, 1H), 7.07 (dd, | |
| | fluorophenyl)-1- | J = 12.0, 2.1 Hz, 1H), 6.99-6.91 (m, 1H), 6.43 | |
| | methyl-1H-pyrrolo[3,2- | (dd, J = 17.0, 10.1 Hz, 1H), 6.27 (dd, J = 17.0, | |
| | c]pyridin-2- | 2.0 Hz, 1H), 5.78 (dd, J = 10.1, 2.0 Hz, 1H), 4.36 | |
| | yl)phenyl)acrylamide | (dt, J = 8.9, 5.0 Hz, 1H), 3.83 (s, 3H), 2.01- | |
| | | 1.83 (m, 2H), 1.70 (dt, J = 7.3, 4.3 Hz, 2H), 1.62- | |
| | | 1.17 (m, 6H). | |

Some compounds of the present invention are listed in the Table 3.

TABLE 3

| Cpds ID | Name | MW |
|---|---|---|
| 542 | N-(4-(4-amino-7-cyano-3-(3-fluoro-4-((6-methylpyridin-2- | 547 |
| | yl)oxy)phenyl)-1-methyl-1H-pyrrolo[3,2-c]pyridin-2-yl)-3- | |
| | methylphenyl)methacrylamide | |
| 543 | N-(4-(4-amino-7-cyano-3-(4-((4-(difluoromethyl)pyrimidin-2- | 566 |
| | yl)oxy)phenyl)-1-methyl-1H-pyrrolo[3,2-c]pyridin-2-yl)-3- | |
| | methylphenyl)methacrylamide | |
| 544 | N-(4-(4-amino-7-cyano-3-(4-((5-fluoro-4-methylpyrimidin-2- | 548 |
| | yl)oxy)phenyl)-1-methyl-1H-pyrrolo[3,2-c]pyridin-2-yl)-3- | |
| | methylphenyl)methacrylamide | |
| 545 | N-(4-(4-amino-7-cyano-1-methyl-3-(4-((1-methyl-1H-pyrazol-3- | 518 |
| | yl)oxy)phenyl)-1H-pyrrolo[3,2-c]pyridin-2-yl)-3- | |
| | methylphenyl)methacrylamide | |
| 546 | N-(4-(4-amino-7-cyano-1-methyl-3-(3-methyl-4-((4-methylpyrimidin- | 544 |
| | 2-yl)oxy)phenyl)-1H-pyrrolo[3,2-c]pyridin-2-yl)-3- | |
| | methylphenyl)methacrylamide | |
| 547 | N-(4-(4-amino-7-cyano-1-methyl-3-(4-((6-methylpyridin-2- | 529 |
| | yl)oxy)phenyl)-1H-pyrrolo[3,2-c]pyridin-2-yl)-3- | |
| | methylphenyl)methacrylamide | |
| 548 | N-(4-(4-amino-7-cyano-3-(4-((4-(difluoromethyl)pyrimidin-2-yl)oxy)- | 604 |
| | 3-fluorophenyl)-1-methyl-1H-pyrrolo[3,2-c]pyridin-2-yl)-3- | |
| | chlorophenyl)methacrylamide | |
| 549 | N-(4-(4-amino-7-cyano-3-(3-fluoro-4-((5-fluoro-4-methylpyrimidin-2- | 586 |
| | yl)oxy)phenyl)-1-methyl-1H-pyrrolo[3,2-c]pyridin-2-yl)-3- | |
| | chlorophenyl)methacrylamide | |
| 550 | N-(4-(4-amino-7-cyano-3-(3-fluoro-4-((4-(trifluoromethyl)pyrimidin- | 622 |
| | 2-yl)oxy)phenyl)-1-methyl-1H-pyrrolo[3,2-c]pyridin-2-yl)-3- | |
| | chlorophenyl)methacrylamide | |
| 551 | N-(4-(4-amino-7-cyano-1-methyl-3-(4-((1-methyl-1H-pyrazol-3- | 522 |
| | yl)oxy)phenyl)-1H-pyrrolo[3,2-c]pyridin-2-yl)-3-methylphenyl)-2- | |
| | fluoroacrylamide | |
| 552 | N-(4-(4-amino-7-cyano-3-(4-((5-fluoro-4-methylpyrimidin-2- | 552 |
| | yl)oxy)phenyl)-1-methyl-1H-pyrrolo[3,2-c]pyridin-2-yl)-3- | |
| | methylphenyl)-2-fluoroacrylamide | |
| 553 | N-(4-(4-amino-7-cyano-3-(4-((4-(difluoromethyl)pyrimidin-2- | 570 |
| | yl)oxy)phenyl)-1-methyl-1H-pyrrolo[3,2-c]pyridin-2-yl)-3- | |
| | methylphenyl)-2-fluoroacrylamide | |
| 554 | N-(4-(4-amino-7-cyano-1-methyl-3-(4-((4-methylpyrimidin-2- | 534 |
| | yl)oxy)phenyl)-1H-pyrrolo[3,2-c]pyridin-2-yl)-3-methylphenyl)-2- | |
| | fluoroacrylamide | |
| 555 | N-(4-(4-amino-7-cyano-1-methyl-3-(4-((6-methylpyridin-2- | 533 |
| | yl)oxy)phenyl)-1H-pyrrolo[3,2-c]pyridin-2-yl)-3-methylphenyl)-2- | |
| | fluoroacrylamide | |
| 556 | N-(4-(4-amino-7-cyano-1-methyl-3-(4-((1-methyl-1H-pyrazol-3- | 542 |
| | yl)oxy)phenyl)-1H-pyrrolo[3,2-c]pyridin-2-yl)-3-chlorophenyl)-2- | |
| | fluoroacrylamide | |
| 557 | N-(4-(4-amino-7-cyano-3-(4-((5-fluoro-4-methylpyrimidin-2- | 572 |
| | yl)oxy)phenyl)-1-methyl-1H-pyrrolo[3,2-c]pyridin-2-yl)-3- | |
| | chlorophenyl)-2-fluoroacrylamide | |
| 558 | N-(4-(4-amino-7-cyano-3-(4-((4-(difluoromethyl)pyrimidin-2- | 590 |
| | yl)oxy)phenyl)-1-methyl-1H-pyrrolo[3,2-c]pyridin-2-yl)-3- | |
| | chlorophenyl)-2-fluoroacrylamide | |
| 559 | N-(4-(4-amino-7-cyano-1-methyl-3-(4-((4-methylpyrimidin-2- | 554 |
| | yl)oxy)phenyl)-1H-pyrrolo[3,2-c]pyridin-2-yl)-3-chlorophenyl)-2- | |
| | fluoroacrylamide | |
| 560 | N-(4-(4-amino-7-cyano-1-methyl-3-(4-((6-methylpyridin-2- | 553 |
| | yl)oxy)phenyl)-1H-pyrrolo[3,2-c]pyridin-2-yl)-3-chlorophenyl)-2- | |
| | fluoroacrylamide | |
| 561 | N-(4-(4-amino-7-cyano-1-methyl-3-(4-((1-methyl-1H-pyrazol-3- | 526 |
| | yl)oxy)phenyl)-1H-pyrrolo[3,2-c]pyridin-2-yl)-3-fluorophenyl)-2- | |
| | fluoroacrylamide | |
| 562 | N-(4-(4-amino-7-cyano-3-(4-((5-fluoro-4-methylpyrimidin-2- | 556 |
| | yl)oxy)phenyl)-1-methyl-1H-pyrrolo[3,2-c]pyridin-2-yl)-3- | |
| | fluorophenyl)-2-fluoroacrylamide | |
| 563 | N-(4-(4-amino-7-cyano-3-(4-((4-(difluoromethyl)pyrimidin-2- | 574 |
| | yl)oxy)phenyl)-1-methyl-1H-pyrrolo[3,2-c]pyridin-2-yl)-3- | |
| | fluorophenyl)-2-fluoroacrylamide | |
| 564 | N-(4-(4-amino-7-cyano-1-methyl-3-(4-((4-methylpyrimidin-2- | 538 |
| | yl)oxy)phenyl)-1H-pyrrolo[3,2-c]pyridin-2-yl)-3-fluorophenyl)-2- | |
| | fluoroacrylamide | |
| 565 | N-(4-(4-amino-7-cyano-1-methyl-3-(4-((6-methylpyridin-2- | 537 |
| | yl)oxy)phenyl)-1H-pyrrolo[3,2-c]pyridin-2-yl)-3-fluorophenyl)-2- | |
| | fluoroacrylamide | |
| 566 | N-(4-(4-amino-7-cyano-1-methyl-3-(4-((1-methyl-1H-pyrazol-3- | 504 |
| | yl)oxy)phenyl)-1H-pyrrolo[3,2-c]pyridin-2-yl)-3- | |
| | methylphenyl)acrylamide | |
| 567 | N-(4-(4-amino-7-cyano-3-(4-((4-(difluoromethyl)pyrimidin-2- | 552 |
| | y1)oxy)phenyl)-1-methyl-1H-pyrrolo[3,2-c]pyridin-2-yl)-3- | |
| | methylphenyl)acrylamide | |
| 568 | N-(4-(4-amino-7-cyano-1-methyl-3-(4-((4-methylpyrimidin-2- | 516 |
| | yl)oxy)phenyl)-1H-pyrrolo[3,2-c]pyridin-2-yl)-3- | |
| | methylphenyl)acrylamide | |
| 569 | N-(4-(4-amino-7-cyano-1-methyl-3-(4-((6-methylpyridin-2- | 515 |
| | yl)oxy)phenyl)-1H-pyrrolo[3,2-c]pyridin-2-yl)-3- | |
| | methylphenyl)acrylamide | |
| 570 | N-(4-(4-amino-7-cyano-1-methyl-3-(4-((1-methyl-1H-pyrazol-3- | 524 |
| | yl)oxy)phenyl)-1H-pyrrolo[3,2-c]pyridin-2-yl)-3- | |
| | chlorophenyl)acrylamide | |
| 571 | N-(4-(4-amino-7-cyano-3-(4-((4-(difluoromethyl)pyrimidin-2- | 572 |
| | yl)oxy)phenyl)-1-methyl-1H-pyrrolo[3,2-c]pyridin-2-yl)-3- | |
| | chlorophenyl)acrylamide | |
| 572 | N-(4-(4-amino-7-cyano-1-methyl-3-(4-((4-methylpyrimidin-2- | 536 |
| | yl)oxy)phenyl)-1H-pyrrolo[3,2-c]pyridin-2-yl)-3- | |
| | chlorophenyl)acrylamide | |
| 573 | N-(4-(4-amino-7-cyano-1-methyl-3-(4-((6-methylpyridin-2- | 535 |
| | yl)oxy)phenyl)-1H-pyrrolo[3,2-c]pyridin-2-yl)-3- | |
| | chlorophenyl)acrylamide | |
| 574 | N-(4-(4-amino-7-cyano-1-methyl-3-(4-((1-methyl-1H-pyrazol-3- | 508 |
| | yl)oxy)phenyl)-1H-pyrrolo[3,2-c]pyridin-2-yl)-3- | |
| | fluorophenyl)acrylamide | |
| 575 | N-(4-(4-amino-7-cyano-1-methyl-3-(4-((6-methylpyridin-2- | 519 |
| | yl)oxy)phenyl)-1H-pyrrolo[3,2-c]pyridin-2-yl)-3- | |
| | fluorophenyl)acrylamide | |
| 576 | N-(4-(4-amino-7-cyano-3-(4-((5-fluoro-4-methylpyrimidin-2- | 550 |
| | yl)oxy)phenyl)-1-methyl-1H-pyrrolo[3,2-c]pyridin-2-yl)phenyl)-2- | |
| | methylprop-2-enethioamide | |
| 577 | N-(4-(4-amino-7-cyano-3-(4-((4-methylpyrimidin-2-yl)oxy)phenyl)- | 536 |
| | 1H-pyrrolo[3,2-c]pyridin-2-yl)-3-chlorophenyl)methacrylamide | |
| 578 | N-(4-(4-amino-7-cyano-1-ethyl-3-(4-((4-methylpyrimidin-2- | 564 |
| | yl)oxy)phenyl)-1H-pyrrolo[3,2-c]pyridin-2-yl)-3- | |
| | chlorophenyl)methacrylamide | |
| 579 | N-(4-(4-amino-7-cyano-1-(2-fluoroethyl)-3-(4-((4-methylpyrimidin-2- | 582 |
| | yl)oxy)phenyl)-1H-pyrrolo[3,2-c]pyridin-2-yl)-3- | |
| | chlorophenyl)methacrylamide | |
| 580 | N-(4-(4-amino-7-cyano-3-(4-((4-methylpyrimidin-2-yl)oxy)phenyl)-1- | 618 |
| | (2,2,2-trifluoroethyl)-1H-pyrrolo[3,2-c]pyridin-2-yl)-3- | |
| | chlorophenyl)methacrylamide | |
| 581 | N-(4-(4-amino-7-cyano-3-(4-((4-(difluoromethyl)-5-fluoropyrimidin- | 556 |
| | 2-yl)oxy)phenyl)-1-methyl-1H-pyrrolo[3,2-c]pyridin-2- | |
| | yl)phenyl)acrylamide | |
| 582 | N-(4-(4-amino-7-cyano-3-(4-((4-(difluoromethyl)-5-fluoropyrimidin- | 574 |
| | 2-yl)oxy)-3-fluorophenyl)-1-methyl-1H-pyrrolo[3,2-c]pyridin-2- | |
| | yl)phenyl)acrylamide | |
| 583 | N-(4-(4-amino-3-(3-chloro-5-fluoro-4-((5-fluoro-4-methylpyrimidin-2- | 572 |
| | yl)oxy)phenyl)-7-cyano-1-methyl-1H-pyrrolo[3,2-c]pyridin-2- | |
| | yl)phenyl)acrylamide | |
| 584 | N-(4-(4-amino-7-cyano-3-(3-fluoro-4-((1-(2,2,2-trifluoroethyl)-1H- | 576 |
| | pyrazol-3-yl)oxy)phenyl)-1-methyl-1H-pyrrolo[3,2-c]pyridin-2- | |
| | yl)phenyl)acrylamide | |
| 585 | N-(4-(4-amino-7-cyano-3-(3-fluoro-4-((4-methylpyrimidin-2- | 570 |
| | yl)oxy)phenyl)-1-methyl-1H-pyrrolo[3,2-c]pyridin-2-yl)-3,5- | |
| | difluorophenyl)methacrylamide | |
| 586 | N-(4-(4-amino-7-cyano-3-(3-fluoro-4-((4-methylpyrimidin-2- | 566 |
| | yl)oxy)phenyl)-1-methyl-1H-pyrrolo[3,2-c]pyridin-2-yl)-3-fluoro-5- | |
| | methylphenyl)methacrylamide | |
| 587 | N-(4-(4-amino-7-cyano-1-methyl-3-(4-((4-methylpyrimidin-2- | 584 |
| | yl)oxy)phenyl)-1H-pyrrolo[3,2-c]pyridin-2-yl)-3- | |
| | (trifluoromethyl)phenyl)methacrylamide | |
| 588 | N-(4-(4-amino-3-(4-((5-chloro-4-methylpyrimidin-2-yl)oxy)phenyl)-7- | 536 |
| | cyano-1-methyl-1H-pyrrolo[3,2-c]pyridin-2-yl)phenyl)acrylamide | |
| 589 | N-(4-(4-amino-3-(4-((5-chloro-4-methylpyrimidin-2-yl)oxy)-3,5- | 572 |
| | difluorophenyl)-7-cyano-1-methyl-1H-pyrrolo[3,2-c]pyridin-2- | |
| | yl)phenyl)acrylamide | |
| 590 | N-(4-(4-amino-3-(4-((5-chloro-4-(trifluoromethyl)pyrimidin-2- | 590 |
| | yl)oxy)phenyl)-7-cyano-1-methyl-1H-pyrrolo[3,2-c]pyridin-2- | |
| | yl)phenyl)acrylamide | |
| 591 | N-(4-(4-amino-3-(4-((5-chloro-4-(trifluoromethyl)pyrimidin-2-yl)oxy)- | 608 |
| | 3-fluorophenyl)-7-cyano-1-methyl-1H-pyrrolo[3,2-c]pyridin-2- | |
| | yl)phenyl)acrylamide | |
| 592 | N-(4-(4-amino-3-(4-((5-chloro-4-methylpyrimidin-2-yl)oxy)phenyl)-7- | 584 |
| | cyano-1-methyl-1H-pyrrolo[3,2-c]pyridin-2-yl)-3- | |
| | chlorophenyl)methacrylamide | |
| 593 | N-(4-(4-amino-7-cyano-1-methyl-3-(4-((4-(trifluoromethyl)pyrimidin- | 604 |
| | 2-yl)oxy)phenyl)-1H-pyrrolo[3,2-c]pyridin-2-yl)-3- | |
| | chlorophenyl)methacrylamide | |
| 594 | N-(4-(4-amino-3-(4-((5-chloro-4-methylpyrimidin-2-yl)oxy)phenyl)-7- | 564 |
| | cyano-1-methyl-1H-pyrrolo[3,2-c]pyridin-2-yl)-3- | |
| | methylphenyl)methacrylamide | |
| 595 | N-(4-(4-amino-7-cyano-1-methyl-3-(4-((4-(trifluoromethyl)pyrimidin- | 584 |
| | 2-yl)oxy)phenyl)-1H-pyrrolo[3,2-c]pyridin-2-yl)-3- | |
| | methylphenyl)methacrylamide | |
| 596 | N-(4-(4-amino-7-cyano-3-(3-fluoro-4-((4-(trifluoromethyl)pyrimidin- | 602 |
| | 2-yl)oxy)phenyl)-1-methyl-1H-pyrrolo[3,2-c]pyridin-2-yl)-3- | |
| | methylphenyl)methacrylamide | |
| 597 | N-(4-(4-amino-3-(4-((5-chloro-4-methylpyrimidin-2-yl)oxy)-3- | 566 |
| | fluorophenyl)-7-cyano-1-methyl-1H-pyrrolo[3,2-c]pyridin-2- | |
| | yl)phenyl)but-2-ynamide | |
| 598 | N-(4-(4-amino-3-(4-((5-chloro-4-(difluoromethyl)pyrimidin-2- | 572 |
| | yl)oxy)phenyl)-7-cyano-1-methyl-1H-pyrrolo[3,2-c]pyridin-2- | |
| | yl)phenyl)acrylamide | |
| 599 | N-(4-(4-amino-3-(4-((5-chloro-4-(difluoromethyl)pyrimidin-2-yl)oxy)- | 590 |
| | 3-fluorophenyl)-7-cyano-1-methyl-1H-pyrrolo[3,2-c]pyridin-2- | |
| | yl)phenyl)acrylamide | |
| 600 | N-(4-(4-amino-3-(4-((5-chloro-4-methylpyrimidin-2-yl)oxy)-3- | 572 |
| | fluorophenyl)-7-cyano-1-methyl-1H-pyrrolo[3,2-c]pyridin-2- | |
| | yl)phenyl)-2-fluoroacrylamide | |
| 601 | N-(4-(4-amino-3-(4-((5-chloro-4-methylpyrimidin-2-yl)oxy)phenyl)-7- | 554 |
| | cyano-1-methyl-1H-pyrrolo[3,2-c]pyridin-2-yl)phenyl)-2- | |
| | fluoroacrylamide | |
| 602 | N-(4-(4-amino-3-(4-((5-chloro-6-methylpyridin-2-yl)oxy)phenyl)-7- | 535 |
| | cyano-1-methyl-1H-pyrrolo[3,2-c]pyridin-2-yl)phenyl)acrylamide | |
| 603 | N-(4-(4-amino-3-(4-((5-chloro-6-methylpyridin-2-yl)oxy)-3- | 553 |
| | fluorophenyl)-7-cyano-1-methyl-1H-pyrrolo[3,2-c]pyridin-2- | |
| | yl)phenyl)acrylamide | |
| 604 | N-(4-(4-amino-3-(3-chloro-4-((5-chloro-4-methylpyrimidin-2- | 570 |
| | yl)oxy)phenyl)-7-cyano-1-methyl-1H-pyrrolo[3,2-c]pyridin-2- | |
| | yl)phenyl)acrylamide | |
| 605 | N-(4-(4-amino-3-(4-((5-chloro-4-methylpyrimidin-2-yl)oxy)-3- | 550 |
| | methylphenyl)-7-cyano-1-methyl-1H-pyrrolo[3,2-c]pyridin-2- | |
| | yl)phenyl)acrylamide | |
| 606 | N-(4-(4-amino-3-(4-((5-chloro-4-methylpyrimidin-2-yl)oxy)-3- | 602 |
| | fluorophenyl)-7-cyano-1-methyl-1H-pyrrolo[3,2-c]pyridin-2-yl)-3- | |
| | chlorophenyl)methacrylamide | |
| 607 | N-(4-(4-amino-3-(4-((5-chloro-4-methylpyrimidin-2-yl)oxy)-3- | 568 |
| | fluorophenyl)-7-cyano-1-methyl-1H-pyrrolo[3,2-c]pyridin-2-yl)-3- | |
| | methylphenyl)acrylamide | |
| 608 | N-(4-(4-amino-3-(4-((5-chloro-4-methylpyrimidin-2-yl)oxy)phenyl)-7- | 550 |
| | cyano-1-methyl-1H-pyrrolo[3,2-c]pyridin-2-yl)-3- | |
| | methylphenyl)acrylamide | |
| 609 | N-(4-(4-amino-3-(4-((5-chloro-4-methylpyrimidin-2-yl)oxy)-3- | 586 |
| | fluorophenyl)-7-cyano-1-methyl-1H-pyrrolo[3,2-c]pyridin-2-yl)-3- | |
| | methylphenyl)-2-fluoroacrylamide | |
| 610 | N-(4-(4-amino-3-(4-((5-chloro-4-methylpyrimidin-2-yl)oxy)phenyl)-7- | 568 |
| | cyano-1-methyl-1H-pyrrolo[3,2-c]pyridin-2-yl)-3-methylphenyl)-2- | |
| | fluoroacrylamide | |
| 611 | N-(4-(4-amino-3-(4-((5-chloro-4-methylpyrimidin-2-yl)oxy)-3- | 588 |
| | fluorophenyl)-7-cyano-1-methyl-1H-pyrrolo[3,2-c]pyridin-2-yl)-3- | |
| | chlorophenyl)acrylamide | |
| 612 | N-(4-(4-amino-3-(4-((5-chloro-4-methylpyrimidin-2-yl)oxy)phenyl)-7- | 570 |
| | cyano-1-methyl-1H-pyrrolo[3,2-c]pyridin-2-yl)-3- | |
| | chlorophenyl)acrylamide | |
| 613 | N-(4-(4-amino-3-(4-((5-chloro-4-methylpyrimidin-2-yl)oxy)-3- | 606 |
| | fluorophenyl)-7-cyano-1-methyl-1H-pyrrolo[3,2-c]pyridin-2-yl)-3- | |
| | chlorophenyl)-2-fluoroacrylamide | |
| 614 | N-(4-(4-amino-3-(4-((5-chloro-4-methylpyrimidin-2-yl)oxy)phenyl)-7- | 588 |
| | cyano-1-methyl-1H-pyrrolo[3,2-c]pyridin-2-yl)-3-chlorophenyl)-2- | |
| | fluoroacrylamide | |
| 615 | N-(4-(4-amino-3-(4-((5-chloro-4-methylpyrimidin-2-yl)oxy)-3- | 572 |
| | fluorophenyl)-7-cyano-1-methyl-1H-pyrrolo[3,2-c]pyridin-2-yl)-3- | |
| | fluorophenyl)acrylamide | |
| 616 | N-(4-(4-amino-3-(4-((5-chloro-4-methylpyrimidin-2-yl)oxy)phenyl)-7- | 554 |
| | cyano-1-methyl-1H-pyrrolo[3,2-c]pyridin-2-yl)-3- | |
| | fluorophenyl)acrylamide | |
| 617 | N-(4-(4-amino-3-(4-((5-chloro-4-methylpyrimidin-2-yl)oxy)-3- | 590 |
| | fluorophenyl)-7-cyano-1-methyl-1H-pyrrolo[3,2-c]pyridin-2-yl)-3- | |
| | fluorophenyl)-2-fluoroacrylamide | |
| 618 | N-(4-(4-amino-3-(4-((5-chloro-4-methylpyrimidin-2-yl)oxy)phenyl)-7- | 572 |
| | cyano-1-methyl-1H-pyrrolo[3,2-c]pyridin-2-yl)-3-fluorophenyl)-2- | |
| | fluoroacrylamide | |

### Biochemical and Cellular Activity

### Example A: Biochemical Caliper Assay

Compounds of the present invention were tested in a FGFR1, FGFR2, and FGFR3 Biochemical Caliper Assay.

Enzyme activity was monitored using KinEASE-TK kit (CisBio, Cat.No. 62TK0PEC) according to manufacturer's instructions. Proteins were purchased from Carna (FGFR1, Cat.No. 08-133; FGFR2, Cat.No. 08-134; FGFR3, Cat.No. 08-135). Final enzyme concentration was 0.2 nM for FGFR1 and FGFR3, 0.07 nM for FGFR2. Compounds were prepared in 10 mM DMSO and serially diluted into 10 concentrations by 3-fold dilution. Prior to the initiation of the reaction by adding TK-substrate/ATP mixture, the compounds were incubated with protein for 10 min at RT. Reaction was proceeded for 45 min at RT. Plates were quenched by adding TK-antibody-Cryptate and Sa-XL665 mixture. After 60 min in the stop solution, read the signal on microplate reader (Molecular Devices, i3x), excitation of 320 nm and emission of 615 nm and 665 nm with a 90 s delay. IC50 values were calculated using Prism 7 (GraphPad).

Results of FGFR2 Biochemical Caliper Assay and the Selectivity of FGFR1/FGFR2 and FGFR3/FGFR2 are shown in Table 4.

Compounds having an IC50 of FGFR2 less or equal to 5 nM are represented as “A+”, Compounds having an IC50 of FGFR2 greater than 5 nM but less than or equal to 10 nM are represented as “A”, Compounds having an IC50 of FGFR2 greater than 10 nM are represented as “B”.

Compounds having a Selectivity of FGFR1/FGFR2 greater or equal to 500 are represented as “A+”, Compounds having a Selectivity of FGFR1/FGFR2 less than 500 but greater than or equal to 300 are represented as “A”, Compounds having a Selectivity of FGFR1/FGFR2 less than 300 but greater than or equal to 200 are represented as “B”, Compounds having a Selectivity of FGFR1/FGFR2 less than 200 but greater than or equal to 100 are represented as “C”.

Compounds having a Selectivity of FGFR3/FGFR2 greater or equal to 150 are represented as “A+”, Compounds having a Selectivity of FGFR3/FGFR2 less than 150 but greater than or equal to 100 are represented as “A”, Compounds having a Selectivity of FGFR3/FGFR2 less than 100 but greater than or equal to 25 are represented as “B”.

### Example B: Cell Line Proliferation Assay

Compounds of the present invention were also tested in a SNU16 Cancer Cell Line Proliferation Assay.

SNU16 cells were plated in 96-well clear bottom/white plate(Costar, Cat.No. 3610) at 5000 cells/well in 100 μl medium. Test compounds were prepared in a 10 mM DMSO stock solution and serially diluted into 9 concentrations by 3-fold dilution. 0.5 μl of each concentration of compound was added into the cell culture plates and DMSO as a control. The plates were incubated for 72 h at 37° C. and 5% CO₂ incubator. 50 μl CellTiter-Glo reagent (Promega, G7572) was added into each well and then the plates were shaken gently for 10 min at room temperature. Luminescence was read on microplate reader (Molecular Devices, i3x), IC50 values were calculated using Prism 7 (GraphPad).

Results of the SNU16 Cancer Cell Line Proliferation Assay are shown in Table 4.

Compounds having an IC50 less or equal to 10 nM are represented as “A+”, Compounds having an IC50 greater than 10 nM but less than or equal to 50 nM are represented as “A”, Compounds having an IC50 greater than 50 nM are represented as “B”.

TABLE 4

| | | |
|---|---|---|
| FGFR2 | Selectivity | Cell |

| Cpds | Cliper | FGFR1/ | FGFR3/ | SNU-16 |
|---|---|---|---|---|
| ID | IC50 | FGFR2 | FGFR2 | IC50 |
| 0* | B | C | B | A+ |
| 21 | A+ | A+ | A | B |
| 22 | A+ | A | B | A+ |
| 23 | A+ | A+ | A | A+ |
| 26 | A+ | A | B | A+ |
| 28 | A+ | A | B | A+ |
| 42 | A+ | A | A | A+ |
| 45 | A+ | B | B | A+ |
| 47 | A+ | B | B | A+ |
| 48 | A+ | A+ | B | A+ |
| 55 | A+ | A+ | A | A+ |
| 56 | A | A+ | A | A+ |
| 59 | A+ | A | A | A+ |
| 60 | A | A+ | A | A+ |
| 65 | A+ | A | A+ | A+ |
| 69 | A+ | A | B | A+ |
| 74 | B | C | A+ | A+ |
| 81 | A+ | A+ | A+ | A+ |
| 85 | A+ | A+ | A+ | A+ |
| 86 | A+ | A+ | B | A+ |
| 89 | A+ | A+ | A+ | A+ |
| 90 | A+ | A | B | A |
| 93 | B | A+ | A+ | A+ |
| 95 | B | A+ | A+ | A+ |
| 96 | A+ | A+ | A+ | A+ |
| 97 | A | A+ | A+ | A+ |
| 98 | A+ | A+ | A+ | A+ |
| 100 | A+ | A+ | A+ | A+ |
| 101 | A+ | A+ | B | A+ |
| 102 | B | B | B | A+ |
| 104 | A | A+ | A+ | A+ |
| 105 | A+ | A+ | A | A+ |
| 107 | A+ | A+ | A | A+ |
| 108 | A+ | A+ | A | A+ |
| 109 | A+ | A+ | B | A+ |
| 115 | A | C | A+ | A |
| 134 | A+ | C | A+ | B |
| 137 | A | A+ | A+ | A |
| 150 | A+ | A+ | B | A+ |
| 151 | A | A+ | A+ | A+ |
| 153 | A+ | A+ | A+ | A+ |
| 154 | A+ | A+ | A | A+ |
| 155 | A+ | A+ | A | A+ |
| 157 | A+ | B | B | A+ |
| 162 | A+ | A+ | A+ | A+ |
| 163 | A+ | A+ | A+ | A+ |
| 167 | A+ | A | B | A+ |
| 168 | A+ | A+ | A+ | A+ |
| 169 | A+ | A+ | B | A+ |
| 170 | A+ | A+ | B | A+ |
| 171 | A+ | C | B | A+ |
| 184 | A+ | A | A+ | A+ |
| 185 | B | A+ | A+ | A+ |
| 188 | A+ | B | B | A+ |
| 189 | A+ | B | B | A+ |
| 193 | A | A+ | A+ | A+ |
| 194 | A | B | A+ | A+ |
| 196 | A+ | A | A+ | A+ |
| 198 | A+ | A | A+ | A+ |
| 199 | A+ | A+ | A+ | A+ |
| 204 | A+ | A+ | A | A+ |
| 205 | A+ | A+ | B | A+ |
| 228 | A | A+ | A+ | A+ |
| 234 | A+ | A+ | A+ | A+ |
| 235 | A+ | A+ | A+ | A+ |
| 236 | A+ | A+ | A | A+ |
| 237 | A | B | A+ | A+ |
| 248 | A+ | C | A+ | A+ |
| 249 | A+ | C | A+ | A+ |
| 267 | A+ | B | A+ | A+ |
| 268 | A | C | A+ | / |
| 271 | A+ | C | B | A+ |
| 289 | B | C | B | A+ |
| 308 | B | C | A | A+ |
| 309 | A | A+ | A+ | A+ |
| 318 | A+ | C | A | A+ |
| 319 | A+ | A | A+ | A+ |
| 320 | A+ | C | A | / |
| 321 | A | C | A+ | A+ |
| 330 | B | A+ | A+ | A+ |
| 339 | B | C | B | A+ |
| 340 | B | B | A | A+ |
| 342 | A+ | B | A+ | A+ |
| 344 | A+ | A+ | A | A+ |
| 351 | A+ | A+ | B | A+ |
| 355 | A+ | A | A+ | A+ |
| 356 | A+ | A | A | A+ |
| 357 | A+ | A | A+ | A+ |
| 358 | A+ | A+ | A | A+ |
| 359 | B | A+ | B | / |
| 361 | A+ | A | A | A+ |
| 362 | B | A+ | A | / |
| 366 | A+ | A+ | A+ | A+ |
| 372 | A+ | A+ | A+ | A+ |
| 376 | B | A+ | A+ | / |
| 395 | B | A+ | B | A+ |
| 400 | A+ | A+ | A+ | A+ |
| 402 | B | A+ | A+ | A+ |
| 410 | A+ | A | A | B |
| 415 | A+ | B | B | A+ |
| 416 | A+ | B | B | A+ |
| 426 | A+ | A+ | A | A+ |
| 427 | A+ | A+ | A | A+ |
| 428 | B | A+ | A+ | A |
| 429 | A | A+ | A+ | A+ |
| 431 | B | A+ | A+ | A+ |
| 432 | A+ | B | B | A+ |
| 433 | A | A+ | A+ | A+ |
| 438 | A+ | A+ | A+ | A+ |
| 439 | A+ | C | B | A+ |
| 443 | A+ | C | B | A+ |
| 450 | B | A+ | B | A+ |
| 451 | A+ | A | B | A+ |
| 467 | B | A+ | A+ | A+ |
| 468 | B | A+ | A+ | A+ |
| 473 | A+ | A+ | A+ | A+ |
| 474 | A | A+ | A+ | A+ |
| 488 | B | A+ | A+ | A+ |
| 497 | A+ | A+ | A+ | A+ |
| 501 | B | A+ | A+ | A+ |

0* the compound of 0 was disclosed in PCT application whose International Publication Number is WO 2020/231990A1, and the chemical name of compound 0 is N-(4-(4-amino-5-(3-fluoro-4-((4-methylpyrimidin-2-yl)oxy)phenyl)-7-methyl-7H-pyrrolo[2,3-d] pyrimidin-6-yl)phenyl)methacrylamide.

### Example C: Pharmacokinetics in SD Rat

Animal studies were done as approved protocols by the Institutional Animal Care and Use Committee at INVIVO Biotech Co., Ltd.

Animal information: Sprague-Dawley (SD) rat, SPF, 6-8 weeks, Male, weight 200-250 g, purchased from Beijing Vital River Lab (Beijing, People's Republic of China).

Formulation: Prepare 1.5 mg/ml and 0.6 mg/ml compound solution in 50% DMSO+100% Solutol+85% HTPβCD(20% HPβCD) for PO and IV respectively, dosing immediately after preparation.

Administration: PO and IV;

Dose: 15 mg/kg for PG and 3 mg/kg for IV; Collecting plasma at 0.083, 0.25, 0.5, 1, 2, 4, 6, 8, and 24 hrs post dosing and analyze through LC-MS/MS Analyst and Winnonlin to calculate PK parameters (T_{1/2}, Cₘₐₓ and AUC_{0-∞}, IV for Clearance (Cl)).

Results of PK are shown in Table S.

TABLE 5

| Cpds ID | Cl (mL/min/kg) | T_{1/2} (hr) | Cₘₐₓ (ng/mL) | AUC_{0-∞} (hr*ng/mL) |
|---|---|---|---|---|
| 0* | 49 | 1.5 | 244 | 614 |
| 28 | 19 | 0.98 | 509 | 1238 |
| 71 | 18 | 1.8 | 1297 | 3234 |
| 96 | 3.1 | 6 | 621 | 2703 |
| 99 | 18 | 1.4 | 2362 | 3080 |
| 185 | 26 | 3 | 507 | 1546 |
| 193 | 8.4 | 3.6 | 1390 | 4570 |
| 235 | 5.4 | 1.3 | 442 | 2068 |
| 270 | 18 | 2 | 978 | 3384 |
| 302 | 3.4 | 2.7 | 615 | 1511 |
| 308 | 17 | 2.4 | 1183 | 3760 |
| 309 | 11 | 2.6 | 957 | 3099 |
| 310 | 16 | 1.1 | 1188 | 2968 |
| 330 | 6.1 | 2.4 | 509 | 1823 |

0* the compound of 0 was disclosed in PCT application whose International Publication Number is WO 2020/231990A1, and the chemical name of compound 0 is N-(4-(4-amino-5-(3-fluoro-4-((4-methylpyrimidin-2-yl)oxy)phenyl)-7-methyl-7H-pyrrolo[2,3-d]pyrimidin-6-yl)phenyl)methacrylamide.

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

We claim:

1. A compound represented by the Formula (I): 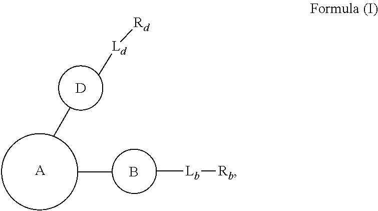
or a pharmaceutically acceptable salt, prodrug, solvate, hydrate, tautomer, and isomer thereof,
wherein:
Ring A is selected from: 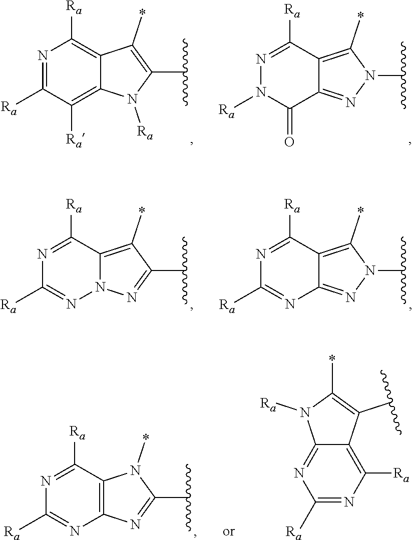
and [symbol] represents a bond to ring D and 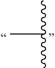
represents a bond to ring B;
Each of Rₐ is independently optionally selected from H, D, CN, NH₂, OH, halogen, C₁₋₆alkyl, C₃₋₆ cycloalkyl, C₁₋₆alkoxyl, —(CH₂)₀₋₂—NHR′, —(CH₂)₀₋₂N(R′)₂, or 3- to 6-membered saturated heterocyclic ring having 1 or 2 heteroatoms optionally independently selected from N, O, or S; and each of which is optionally independently unsubstituted or substituted with 1, 2, or 3 instances of D, OH, halogen, C₁₋₃alkyl, or C₁₋₃alkoxyl;
Ra′ is D, CN, OH, C₁₋₆alkoxyl, —CONH₂, —COR′, —CONHR′, or —CON(R′)₂; and each of which is optionally independently unsubstituted or substituted with 1, 2, or 3 instances of D, OH, halogen, C₁₋₃alkyl, or C₁₋₃alkoxyl;
Or Ra and Ra′ are taken together with their intervening atoms to form a 5- to 7-membered heterocyclic ring having at least one nitrogen atom, each of which is optionally independently unsubstituted or substituted with 1, 2, 3 instance of C₁₋₃alkoxy, C₁₋₃alkyl, D or halogen;
Ring B is phenylene; a bivalent saturated or partially unsaturated 3-10 membered carbocyclic ring; a bivalent saturated or partially unsaturated 3-10 membered heterocyclic ring having 1-5 heteroatoms independently selected from N, O, S, S(O), S(O)₂, S(O)(NH), or S(O)(NC₁₋₃alky); or a 5-10 membered heteroarylene having 1-5 heteroatoms or groups independently selected from N, O, or S; each of which is optionally independently unsubstituted or substituted with r instances of R_{B} in addition to -L_{b}-R_{b}; and r is 0, 1, 2, 3, or 4;
Ring D is phenylene; a bivalent saturated or partially unsaturated 3-10 membered carbocyclic ring; a bivalent saturated or partially unsaturated 3-10 membered heterocyclic ring having 1-5 heteroatoms independently selected from N, O, S, S(O), S(O)₂, S(O)(NH), or S(O)(NC₁₋₃alky); or a 5-10 membered heteroarylene having 1-5 heteroatoms independently selected from N, O, or S; and each of which is optionally independently unsubstituted or substituted with u instances of R_{D} in addition to -L_{d}-R_{d}; and u is 0, 1, 2, 3, or 4;
Each of R_{B} or R_{D} is independently optionally selected from -D, oxo, —OH, —NH₂, —CN, —NO₂, —(CH₂)₀₋₂ NHR′, —(CH₂)₀₋₂N(R′)₂, —(CH₂)₀₋₂OR′, R, —SO₂R, or —COR;
Each of L_{b} and L_{d} is independently optionally selected from a covalent bond, —O—, —S—, —NHS(O)₂—, —S(O)—, S(O)₂—, —CO—, —NH—, —(CH₂)₁₋₃, —CONH—, —C(S)NH—, —NHCO—, —CONH(CH₂)₁₋₃—, —NHCO(CH₂)₁₋₃—, or —NHCONH—; and each of CH₂ is independently optionally unsubstituted or substituted with 1, 2, or 3 instances of D, halogen, C₁₋₃alkyl, or C₁₋₃haloalkyl; each of NH is independently optionally unsubstituted or substituted with C₁₋₃ alkyl;
R_{b} is halogen, CN, 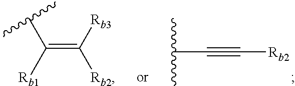
each of R_{b1}, R_{b2}, and R_{b3} is independently optionally selected from H, D, halogen, —CN, C₁₋₆ alkyl, C₃₋₆cycloalkyl, C₂₋₆alkenyl, C₂₋₆alknyl, —(CH₂)₀₋₂NHR′, —(CH₂)₀₋₂N(R′)₂, or —(CH₂)₀₋₂OR′; and each of which is optionally independently unsubstituted or substituted with 1, 2, 3 instances of halogen or D; or
R_{b1} and R_{b2}, R_{b2} and R_{b3}, R_{b1} and L_{b}, are taken with their intervening atoms to form 4-7 membered saturated or partially unsaturated ring having 0-2 heteroatoms or group selected from N, O, S, S(O), or S(O)₂;
R_{d} is H; D; C₁₋₆ alkyl; phenyl; a saturated or partially unsaturated 3-10 membered carbocyclic ring; a saturated or partially unsaturated 3-10 membered heterocyclic ring having 1-5 heteroatoms independently selected from N, O, S, S(O), or S(O)₂; or a 5-10 membered heteroaryl having 1-5 heteroatoms or groups independently selected from N, O, or S; each of which is optionally independently unsubstituted or substituted with 1, 2, 3 instances of R;
Each of R is optionally independently selected from halogen, —CN, —C₁₋₆ alkyl, —C₃₋₆ cycloalkyl —SC₁₋₆ alkyl, —N(C₁₋₆ alkyl)₂, —NHC₁₋₆ alkyl, —NHC₃₋₆ cycloalkyl, —C₁₋₆ alkoxy or —OC₃₋₆ cycloalkyl; and each of which is optionally unsubstituted or substituted with 1, 2, 3 instance of D or halogen;
Each of R′ is optionally independently selected from C₁₋₆alkyl or C₃₋₆cycloalkyl; or
two R′ are taken together with the nitrogen which both attached to form a 4- to 6-membered heterocyclic ring optionally having additional 1 or 2 heteroatoms or group selected from N, O, S, S(O), or S(O)₂.

2. The compound or pharmaceutically acceptable salt thereof of claim 1, wherein the compound represented by the Formula (II):

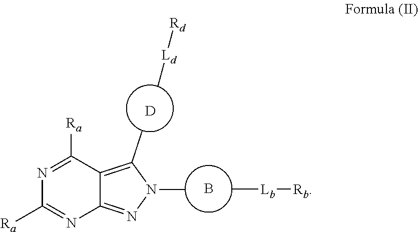

3. The compound or pharmaceutically acceptable salt thereof of claim 1, wherein the compound represented by the Formula (III):

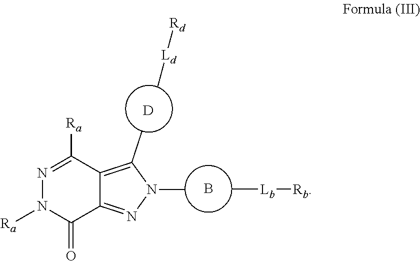

4. The compound or pharmaceutically acceptable salt thereof of claim 1, wherein the compound represented by the Formula (IV):

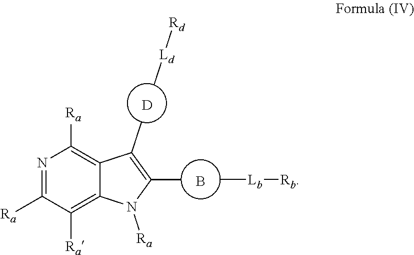

5. The compound or pharmaceutically acceptable salt thereof of claim 1, wherein the compound represented by the Formula (V):

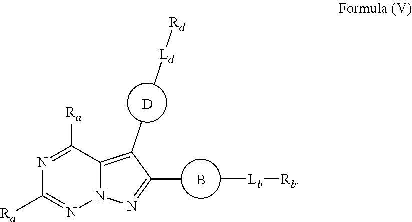

6. The compound or pharmaceutically acceptable salt thereof of claim 1, wherein the compound represented by the Formula (VI)

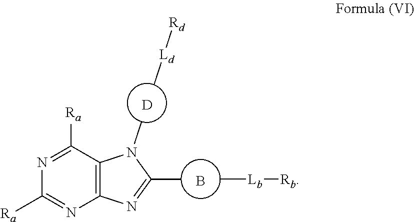

7. The compound or pharmaceutically acceptable salt thereof of claim 1, wherein the compound represented by the Formula (VII):

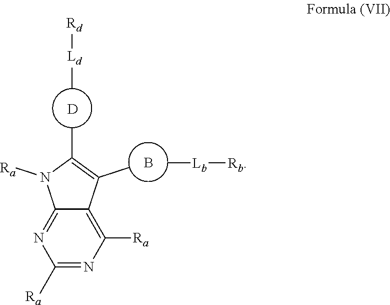

8. The compound or pharmaceutically acceptable salt thereof of any one of claim 1-7, wherein the said each of Ra is independently optionally selected from H, D, CN, NH₂, OH, F, Cl, Br, C₁₋₃alkyl, C₁₋₃alkoxyl, cyclopropyl, cyclobutyl, cyclopentyl, cyclohexyl, —NHR′, —CH₂NHR′, —(CH₂)₂NHR′, —N(R′)₂, —CH₂N(R′)₂, —(CH₂)₂N(R′)₂, 3-membered saturated heterocyclic ring, 4-membered saturated heterocyclic ring, 5-membered saturated heterocyclic ring, or 6-membered saturated heterocyclic ring; furthermore each of heterocyclic ring has 1 or 2 heteroatoms optionally independently selected from N, O, or S; and each of which is optionally independently unsubstituted or substituted with 1, 2, or 3 instances of D, OH, F, Cl, Br, methyl, ethyl, propyl, methoxy, or ethoxy.

9. The compound or pharmaceutically acceptable salt thereof of any one of claim 1-8, wherein, each of Ra is independently optionally selected from H, D, CN, NH₂, OH, F, Cl, methyl, ethyl, propyl, iso-propyl, methoxy, ethoxy, propoxy, cyclopropyl, cyclobutyl, cyclopentyl, cyclohexyl, —NHR′, —CH₂NHR′, —(CH₂)₂NHR′, —N(R′)₂, —CH₂N(R′)₂, —(CH₂)₂N(R′)₂, 4-membered saturated heterocyclic ring, 5-membered saturated heterocyclic ring, or 6-membered saturated heterocyclic ring; furthermore each of saturated heterocyclic ring has 1 or 2 heteroatoms optionally independently selected from N or O; and each of which is optionally independently unsubstituted or substituted with 1, 2, or 3 instances of D, OH, F, Cl, methyl, ethyl, methoxy, or ethoxy.

10. The compound or pharmaceutically acceptable salt thereof of any one of claim 1-9, wherein, the said Rₐ is independently optionally selected from H, D, CN, NH₂, OH, F, methyl, cyclopropyl, cyclobutyl, —NHR′, —CH₂NHR′, —(CH₂)₂NHR′, —N(R′)₂, —CH₂N(R′)₂, —(CH₂)₂N(R′)₂, 4-membered saturated heterocyclic ring, 5-membered saturated heterocyclic ring, or 6-membered saturated heterocyclic ring; furthermore each of heterocyclic ring has 1 or 2 heteroatoms optionally independently selected from N, or O; and each of which is optionally independently unsubstituted or substituted with 1, 2, or 3 instances of D, OH, or F.

11. The compound or pharmaceutically acceptable salt thereof of any one of claim 1-10, wherein, the said each of Ra is

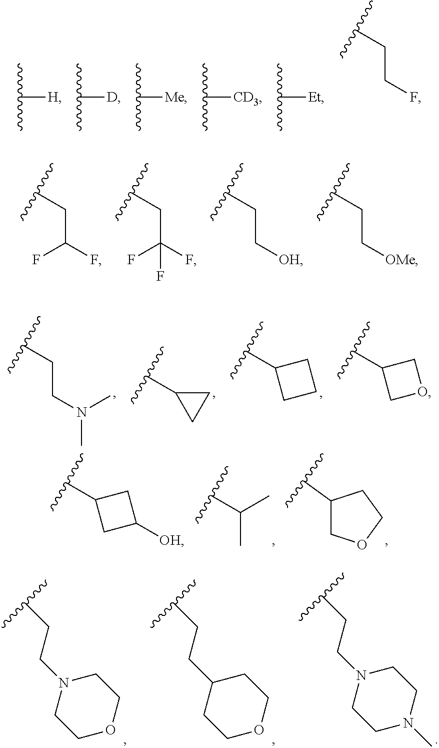

12. The compound or pharmaceutically acceptable salt thereof of any one of claim 1-11, wherein, the said Ra′ is D, CN, OH, C₁₋₃alkoxyl, —CONH₂, —COR′, —CONHR′, or —CON(R′)₂; and each of which is optionally independently unsubstituted or substituted with 1, 2, or 3 instances of D, OH, F, Cl, Br, methyl, ethyl, propyl, iso-propyl, methoxy, ethoxy, or propoxy.

13. The compound or pharmaceutically acceptable salt thereof of any one of claim 1 to claim 12, wherein, the said Ra′ is D, CN, OH,-methoxy, ethoxy, propoxy, —CONH₂, —COR′, —CONHR′, or —CON(R′)₂; and each of which is optionally independently unsubstituted or substituted with 1, 2, or 3 instances of D, OH, F, Cl, methyl, ethyl, methoxy, or ethoxy.

14. The compound or pharmaceutically acceptable salt thereof of any one of claim 1-13, wherein, the said Ra′ is D, CN, or —CONH₂.

15. The compound or pharmaceutically acceptable salt thereof of any one of claim 1-7, wherein, the said Ra and Ra′ are taken together with their intervening atoms to form a 5-membered heterocyclic ring, 6-membered heterocyclic ring, or 7-membered heterocyclic ring, furthermore each of heterocyclic ring also contains optionally one additional heteroatom selected from N or S; and each of which is optionally independently unsubstituted or substituted with 1, 2, 3 instance of methyl, ethyl, methoxy, ethoxy, D, F, or Cl.

16. The compound or pharmaceutically acceptable salt thereof of any one of claim 1-7, or claim 16, wherein, the said Ra and Ra′ are taken together with their intervening atoms to form a 6-membered heterocyclic ring having one N atom or 6-membered heterocyclic ring having one N atom and one O atom; and each of which is optionally independently unsubstituted or substituted with 1, 2, 3 instance of methyl, D, F, or Cl.

17. The compound or pharmaceutically acceptable salt thereof of any one of claim 1-16, wherein, the said ring B is phenylene; a bivalent saturated or partially unsaturated 5- to 9-membered carbocyclic ring; a bivalent saturated or partially unsaturated 5- to 9-membered heterocyclic ring having 1-4 heteroatoms independently selected from N, O, S, S(O), or S(O)₂; or a 5-9 membered heteroarylene having 1-4 heteroatoms independently selected from N, O, or S; each of which is optionally independently unsubstituted or substituted with r instances of R_{B} in addition to -L_{b}-R_{b}.

18. The compound or pharmaceutically acceptable salt thereof of any one of claim 1-18, wherein, the said ring B is phenylene; a bivalent saturated or partially unsaturated 5-, 6-, 7-, 8- or 9-membered carbocyclic ring; a bivalent saturated or partially unsaturated 5-, 6-, 7-, 8-, or 9-membered heterocyclic ring having 1, 2, 3, or 4 heteroatoms independently selected from N, O, or S; or a 5-, 6-, 7- or 8-, or 9-membered heteroarylene having 1, 2, 3, or 4 heteroatoms independently selected from N, O, or S; each of which is optionally independently unsubstituted or substituted with r instances of R_{B} in addition to -L_{b}-R_{b}.

19. The compound or pharmaceutically acceptable salt thereof of any one of claim 1-19, wherein, the said ring B is phenylene; a bivalent saturated 5-, 6-, or 7-membered heterocyclic ring having 1, 2, or 3 heteroatoms independently selected from N or O; or a 5-, 6-, or 7-membered heteroarylene having 1, 2, or 31 or 2 heteroatoms independently selected from N or O; each of which is optionally independently unsubstituted or substituted with r instances of R_{B} in addition to -L_{b}-R_{b}.

20. The compound or pharmaceutically acceptable salt thereof of any one of claim 1-20, wherein, the said ring B is phenylene; a bivalent saturated 5- or 6-membered heterocyclic ring having 1 or 2 heteroatoms independently selected from N; or a 5- or 6-membered heteroarylene having 1 or 2 heteroatoms independently selected from N; each of which is optionally independently unsubstituted or substituted with r instances of R_{B} in addition to -L_{b}-R_{b}.

21. The compound or pharmaceutically acceptable salt thereof of any one of claim 1-20, wherein, the said ring B is phenylene, 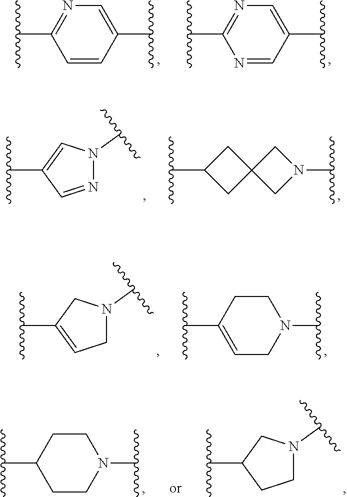
and each of 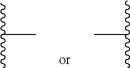
can optionally attach to ring A or -L_{b}-R_{b}; each of which is optionally independently unsubstituted or substituted with r instances of R_{B} in addition to -L_{b}-R_{b}.

22. The compound or pharmaceutically acceptable salt thereof of any one of claim 1-22, wherein, the said ring D is phenylene; a bivalent saturated or partially unsaturated 5- to 9-membered carbocyclic ring; a bivalent saturated or partially unsaturated 5- to 9-membered heterocyclic ring having 1-4 heteroatoms independently selected from N, O, S, S(O), or S(O)₂; or a 5-9 membered heteroarylene having 1-4 heteroatoms independently selected from N, O, or S; and each of which is optionally independently unsubstituted or substituted with u instances of R_{D} in addition to -L_{d}-R_{d}.

23. The compound or pharmaceutically acceptable salt thereof of any one of claim 1-23, wherein, the said ring D is phenylene; a bivalent saturated or partially unsaturated 5-, 6-, 7-, 8- or 9-membered carbocyclic ring; a bivalent saturated or partially unsaturated 5-, 6-, 7-, 8-, or 9-membered heterocyclic ring having 1, 2, 3, or 4 heteroatoms independently selected from N, O, or S; or a 5-, 6-, 7- or 8-, or 9-membered heteroarylene having 1, 2, 3, or 4 heteroatoms independently selected from N, O, or S; and each of which is optionally independently unsubstituted or substituted with u instances of R_{D} in addition to -L_{d}-R_{d}.

24. The compound or pharmaceutically acceptable salt thereof of any one of claim 1-24, wherein, the said ring D is phenylene; a bivalent saturated 5-, 6-, or 7-membered heterocyclic ring having 1, 2, or 3 heteroatoms independently selected from N or O; or a 5-, 6-, or 7-membered heteroarylene having 1, 2, or 31 or 2 heteroatoms independently selected from N or O; and each of which is optionally independently unsubstituted or substituted with u instances of R_{D} in addition to -L_{d}-R_{d}.

25. The compound or pharmaceutically acceptable salt thereof of any one of claim 1-24, wherein, the said ring D is phenylene; a bivalent saturated 5- or 6-membered heterocyclic ring having 1 or 2 heteroatoms independently selected from N; or a 5- or 6-membered heteroarylene having 1 or 2 heteroatoms independently selected from N; and each of which is optionally independently unsubstituted or substituted with u instances of R_{D} in addition to -L_{d}-R_{d}

26. The compound or pharmaceutically acceptable salt thereof of any one of claim 1-25, wherein, the said each of ring D is a phenylene;

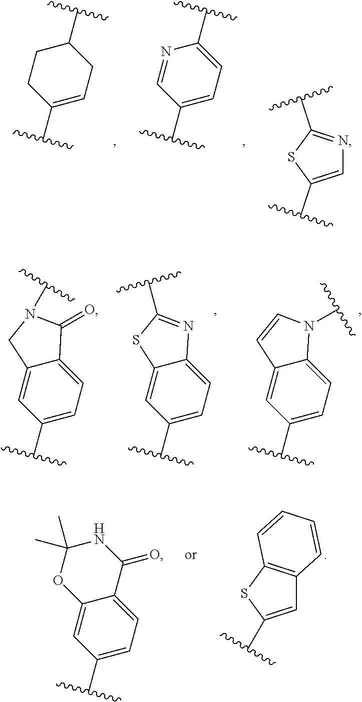

27. The compound or pharmaceutically acceptable salt thereof of any one of claim 1-26, wherein, the said each of R_{B} or R_{D} is independently optionally selected from -D, oxo, —OH, —NH₂, —CN, —NO₂, —NHR′, —CH₂NHR′, —(CH₂)₂NHR′, —N(R′)₂, —CH₂N(R′)₂, —(CH₂)₂N(R′)₂, —OR′, —CH₂OR′, —(CH₂)₂OR′, R, —SO₂R, or —COR.

28. The compound or pharmaceutically acceptable salt thereof of any one of claim 1-27, wherein, the said each of R_{B} or R_{D} is independently optionally selected from -D, oxo, —OH, —NH₂, —CN, —NO₂, R, —NHR′, —CH₂NHR′, —(CH₂)₂NHR′, —N(R′)₂, —CH₂N(R′)₂, or —(CH₂)₂N(R′)₂.

29. The compound or pharmaceutically acceptable salt thereof of any one of claim 1-28, wherein, the said each of R_{B} is independently

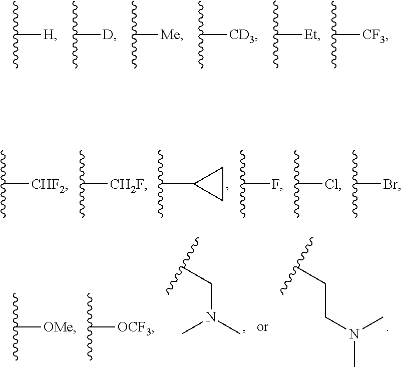

30. The compound or pharmaceutically acceptable salt thereof of any one of claim 1-29, wherein, the said each of R_{D} is independently selected from H, D, F, Cl, CN, OH, methyl, ethyl, propyl, cyclopropyl, -SMe, -OMe, -OEt, —CFH₂, —CF₂H, —CF₃; each of which is independently optionally unsubstituted or substituted with 1, 2, or 3 instances of F, Cl, or D.

31. The compound or pharmaceutically acceptable salt thereof of any one of claim 1-30, wherein, the said each of L_{b} and L_{d} is independently optionally selected from a covalent bond, —O—, —S—, —CO—, —NH—, —CH₂—, —(CH₂)₂, —CONH—, —NHCO—, —CONHCH₂—, —CONH(CH₂)₂—, —NHCO(CH₂)₂—, —NHCOCH₂—, or —NHCONH—; and each of which is independently optionally unsubstituted or substituted with w instances of R_{L}.

32. The compound or pharmaceutically acceptable salt thereof of any one of claim 1-31, wherein, the said each of L_{b} and L_{d} is independently optionally selected from a covalent bond, —O—, —S—, —CO—, —NH—, —CONH—, —NHCO—; and each of which is independently optionally unsubstituted or substituted with 1, 2, or 3 instances of D, F, Cl, Br, methyl, ethyl, propyl, methoxy, ethoxy, propoxy, methyl substituted with F or Cl, ethyl substituted with F or Cl, propyl substituted with F or Cl, methoxy substituted with F or Cl, ethoxy substituted with F or Cl, or propoxy substituted with F or Cl.

33. The compound or pharmaceutically acceptable salt thereof of any one of claim 1-32, wherein, the said each of L_{b} and L_{d} is independently optionally selected from a covalent bond, —O—, —S—, —CO—, —NH—, —CONH—, —NHCO—; and each of which is independently optionally unsubstituted or substituted with 1, 2, or 3 instances of D, F, Cl, methyl, methoxy, methyl substituted with F or Cl, or methoxy substituted with F or Cl.

34. The compound or pharmaceutically acceptable salt thereof of any one of claim 1-33, wherein, the said R_{b} is halogen, CN,

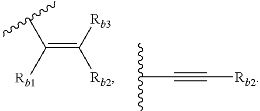

35. The compound or pharmaceutically acceptable salt thereof of any one of claim 1-34, wherein, the said R_{b} is

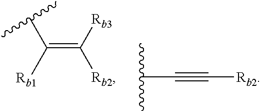

36. The compound or pharmaceutically acceptable salt thereof of any one of claim 1-35, wherein, the said each of R_{b1}, R_{b2}, and R_{b3} is independently optionally selected from H, D, F, Cl, Br, —CN, C₁₋₃ alkyl, C₃₋₆cycloalkyl, C₂₋₃alkenyl, C₂₋₆alknyl, —CH₂NHR′, —(CH₂)₂NHR′, —CH₂N(R′)₂, —(CH₂)₂N(R′)₂, —OCR′—CH₂OCR′, or —(CH₂)₂₀OCR′; wherein, each of which is optionally independently unsubstituted or substituted with 1, 2, 3 instances of F, Cl, Br, or D.

37. The compound or pharmaceutically acceptable salt thereof of any one of claim 1-36, wherein, the said each of R_{b1}, R_{b2}, and R_{b3} is independently optionally selected from H, D, F, Cl, —CN, methyl, ethyl, propyl, i-propylvinyl, ethynyl, or ethynyl; wherein, each of which is optionally independently unsubstituted or substituted with 1, 2, 3 instances of F, Cl, or D.

38. The compound or pharmaceutically acceptable salt thereof of any one of claim 1-37, wherein, the said each of R_{b1}, R_{b2}, and R_{b3} is independently optionally selected from H, D, F, Cl, —CN, or methyl; wherein, each of which is optionally independently unsubstituted or substituted with 1, 2, 3 instances of F or D.

39. The compound or pharmaceutically acceptable salt thereof of any one of claim 1-38, wherein, the said R_{d} is H; D; C₁₋₃ alkyl; phenyl; a 3-, 4-, 5-, 6-, or 7-membered saturated or partially unsaturated monocyclic carbocyclic ring; a 5-, 6-, 7-, 8-, or 9-membered saturated or partially unsaturated bicyclic carbocyclic ring; a 5- or 6-membered monocyclic heteroaryl ring having 1 or 2 heteroatoms independently selected from N, O or S; a 3-, 4-, 5-, 6-, or 7-membered saturated or partially unsaturated monocyclic heterocyclic ring having 1 or 2 heteroatoms independently selected from N, O, or S; or a 5-, 6-, 7-, 8-, or 9-membered saturated or partially unsaturated bicyclic heterocyclic ring having 1 or 2 heteroatoms independently selected from N, O, or S; each of which is optionally independently unsubstituted or substituted with 1, 2, 3 instances of R.

40. The compound or pharmaceutically acceptable salt thereof of any one of claim 1-39, wherein, the said R_{d} is H; D; methyl; ethyl; propyl; i-propyl; phenyl; 5- or 6-membered monocyclic heteroaryl ring having 1 or 2 heteroatoms independently selected from N or S; a 3-, 4-, 5-, 6-, or 7-membered saturated monocyclic carbocyclic ring; a 4-, 5-, or 6-membered saturated monocyclic heterocyclic ring having 1 or 2 heteroatoms independently selected from N, O, or S; or a 5-, 6-, or 7-membered saturated bicyclic carbocyclic ring; each of which is optionally independently unsubstituted or substituted with 1, 2, 3 instances of R.

41. The compound or pharmaceutically acceptable salt thereof of any one of claim 1-40, wherein, the said R_{d} is H; D; methyl; ethyl; phenyl; 5- or 6-membered monocyclic heteroaryl ring having 1 or 2 heteroatoms independently selected from N or S; a 3-, 4-, 5-, or 6-membered saturated carbocyclic ring; a 4-, 5-, or 6-membered saturated monocyclic heterocyclic ring having 1 or 2 heteroatoms independently selected from N; or a 5-, 6-, or 7-membered saturated bicyclic carbocyclic ring; each of which is optionally independently unsubstituted or substituted with 1, 2, 3 instances of R.

42. The compound or pharmaceutically acceptable salt thereof of any one of claim 1-41, wherein, the said R_{d} is 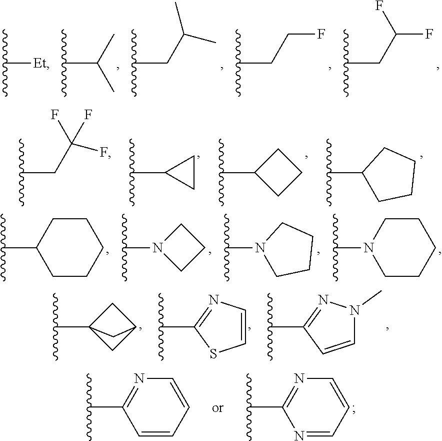
each of which is optionally independently unsubstituted or substituted with 1, 2, 3 instances of R.

43. The compound or pharmaceutically acceptable salt thereof of any one of claim 1-42, wherein, the said R is optionally independently selected from F, Cl, Br, C₁₋₃ alkyl, cyclopropyl, cyclobutyl, cyclopentyl, cyclohexyl, —SC₁₋₃alkyl, —N(C₁₋₃ alkyl)₂, —NHC₁-3alkyl, —NH-cyclopropyl, —NH-cyclobutyl, —NH-cyclopentyl, —NH-cyclohexyl, —C₁₋₃ alkoxy, —O-cyclopropyl, —O-cyclobutyl, —O-cyclopentyl, or —O-cyclohexyl; and each of which is optionally unsubstituted or substituted with 1, 2, 3 instance of D, F, Cl, or Br.

44. The compound or pharmaceutically acceptable salt thereof of any one of claim 1-43, wherein, the said R is optionally independently selected from F, Cl, Br, methyl, ethyl, propyl, i-propyl, methoxy, ethoxy, cyclopropyl, cyclobutyl, cyclopentyl, —NH-cyclobutyl, —C₁₋₃ alkoxy, —O-cyclopropyl or —O-cyclobutyl; and each of which is optionally unsubstituted or substituted with 1, 2, 3 instance of D, F, or Cl.

45. The compound or pharmaceutically acceptable salt thereof of any one of claim 1-44, wherein, the said R is optionally independently selected from F, Cl, methyl, ethyl, methoxy, or cyclopropyl; and each of which is optionally unsubstituted or substituted with 1, 2, 3 instance of D or F.

46. The compound or pharmaceutically acceptable salt thereof of any one of claim 1-45, wherein, the said each of R′ is optionally independently selected from C₁₋₃alkyl, cyclopropyl, cyclobutyl, or cyclopentyl.

47. The compound or pharmaceutically acceptable salt thereof of any one of claim 1-45, wherein, the said each of R′ is optionally independently selected from methyl, ethyl, propyl, i-propyl, cyclopropyl, cyclobutyl, or cyclopentyl.

48. The compound represented by the Formula (I-I) or Formula (I-II): 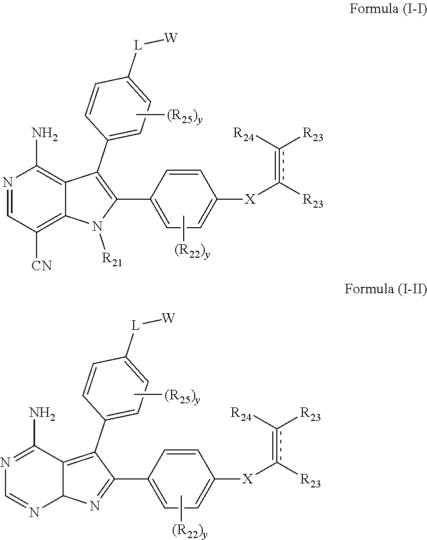
Wherein,
R₂₁ is selected from H, D, CN, C₁₋₃alkyl, C₁₋₃alkoxyl, C₃₋₆cycloalkyl, —(CH₂)₀₋₂—NHC₁₋₃alkyl, —(CH₂)₀₋₂N(C₁₋₃alkyl)₂, or 3- to 6-membered saturated heterocyclic ring having 1 or 2 heteroatoms optionally independently selected from N, O, or S; and each of which is optionally independently unsubstituted or substituted with 1, 2, or 3 instances of D, OH, halogen, C₁₋₃alkyl, C₁₋₃alkoxyl, or C₃₋₆cycloalkyl;
Each of R₂₂ is optionally independently selected from H, D, halogen, CN, NH₂, OH, C₁₋₃alkyl, C₁₋₃alkoxyl, C₃₋₆cycloalkyl, —(CH₂)₀₋₂—NHC₁₋₃alkyl, or —(CH₂)₀₋₂N(C₁₋₃alkyl)₂, and each of which is optionally independently unsubstituted or substituted with 1, 2, or 3 instances of R_{z}; or
R₂₁ and R₂₂ are taken together with their intervening atoms to form a 5- to 7-membered heterocyclic ring having at least one nitrogen atom;
Each of R₂₅ is optionally independently H, D, halogen, CN, OH, R″, N(R″)₂, OR″, or —SR″;
y is 0, 1, 2, 3, or 4;
Each of X or L is optionally independently selected from a covalent bond, —O—, —S—, —S(O)—, —NHS(O)₂—, —S(O)₂—, —CO—, —NH—, —CH₂—, —CONH—, —NHCO—, —CONHCH₂—, —NHCONH—, —NHCOCH₂—, or —NHCOCH₂CH₂—; and each of CH₂ is independently optionally unsubstituted or substituted with 1, 2, or 3 instances of D, halogen, C₁₋₃alkyl, or C₁₋₃haloalkyl; each of NH is independently optionally unsubstituted or substituted with C₁₋₃alkyl; [symbol]
is a bivalent bond or a trivalent bond;
3) when [symbol] is a trivalent bond, each of R₂₃ is absent, and each of R₂₄ is independently optionally selected from H, D, —C₀₋₃ alkylene-N(R″)₂, —C₀₋₃ alkylene-OR″, or R″; each of which is optionally unsubstituted or substituted with 1, 2, or 3 instances of D or halogen;
4) when [symbol] is a bivalent bond, each of R₂₃ or R₂₄ is independently optionally selected from H, D, halogen, —C₀₋₃alkylene-N(R″)₂, —C₀₋₃ alkylene-OR″, or R″; each of which is optionally unsubstituted or substituted with 1, 2, or 3 instances of D or halogen;
Each of W is optionally independently selected from C₁₋₃alkyl, phenyl, 4- to 6-membered saturated or partially saturated heterocyclic ring having 1 or 2 heteroatoms selected from N, O, or S, 5- or 6-membered heteroaryl having 1 to 3 heteroatoms selected from N, O, or S, or 3- to 6-membered saturated or partially saturated carbocyclic ring; each of which is optionally independently unsubstituted or substituted with 1, 2, or 3 instances of R_{w};
Each of R_{w} is optionally independently selected from D, halogen, OH, CN, NH₂, C₁₋₃alkyl, C₁₋₃ haloalkyl, C₃₋₆ cycloalkyl, C₂₋₃alkenyl or C₂₋₃alknyl; and each of which is optionally independently unsubstituted or substituted with 1, 2, or 3 instances of R_{z};
Each of R_{z} is optionally independently selected from H, D, halogen, CN, —N(R″)₂, —OR″, or R″;
Each of R″ is optionally independently C₁₋₃ alkyl, or C₃₋₆ cycloalkyl, each of which is optionally unsubstituted or substituted with 1, 2, or 3 instances of halogen or D;

49. The compound or pharmaceutically acceptable salt thereof of claim 48, wherein, the said R₂₁ is selected from H, D, CN, NH₂, OH, methyl, ethyl, propyl, i-propyl, methoxy, ethoxy, cyclopropyl, cyclobutyl, cyclopentyl, cyclohexyl, —(CH₂)₀₋₂—NHCH₃, —(CH₂)₀₋₂—NHCH₂CH₃, —(CH₂)₀₋₂N(CH₃)₂, —(CH₂)₀₋₂N(CH₂CH₃)₂, —(CH₂)₀₋₂N(CH₃)(CH₂CH₃), 4-membered saturated heterocyclic ring, 5-membered saturated heterocyclic ring, or 6-membered saturated heterocyclic ring having one or two heteroatom selected from N or O; and each of which is optionally independently unsubstituted or substituted with 1, 2, or 3 instances of D, OH, F, Cl, Br, methoxy, ethoxy, or propoxy; the said R₂₂ is optionally independently selected from H, D, F, Cl, Br, CN, NH₂, OH, methyl, ethyl, propyl, i-propyl, methoxy, ethoxy, cyclopropyl, cyclobutyl, cyclopentyl, cyclohexyl, —(CH₂)₀₋₂—NHCH₃, —(CH₂)₀₋₂—NHCH₂CH₃, —(CH₂)₀₋₂N(CH₃)₂, —(CH₂)₀₋₂N(CH₂CH₃)₂, or —(CH₂)₀₋₂N(CH₃CH₂CH₃); and each of which is optionally independently unsubstituted or substituted with 1, 2, or 3 instances of R_{z}.

50. The compound or pharmaceutically acceptable salt thereof of claim 76, wherein, the said R₂₁ is selected from H, D, CN, NH₂, OH, methyl, ethyl, methoxy, cyclopropyl, cyclohexyl, 4-membered saturated heterocyclic ring, or 5-membered saturated heterocyclic ring having one or two heteroatom selected from N or O; and each of which is optionally independently unsubstituted or substituted with 1, 2, or 3 instances of D, OH, F, or Cl; and each of R₂₂ is selected from H, D, F, Cl, CN, NH₂, OH, methyl, ethyl, methoxy, ethoxy, cyclopropyl, cyclobutyl, —NHCH₃, —CH₂NHCH₃, —CH₂CH₂NHCH₃, —CH₂NHCH₂CH₃, —N(CH₃)₂, —CH₂N(CH₃)₂, —(CH₂)₂N(CH₃)₂, —N(CH₃CH₂CH₃), —CH₂N(CH₃CH₂CH₃), or —(CH₂)₂N(CH₃CH₂CH₃); and each of which is optionally independently unsubstituted or substituted with 1, 2, or 3 instances of R_{z}.

51. The compound or pharmaceutically acceptable salt thereof of any one of claim 48-50, wherein, the said R₂₁ is selected from

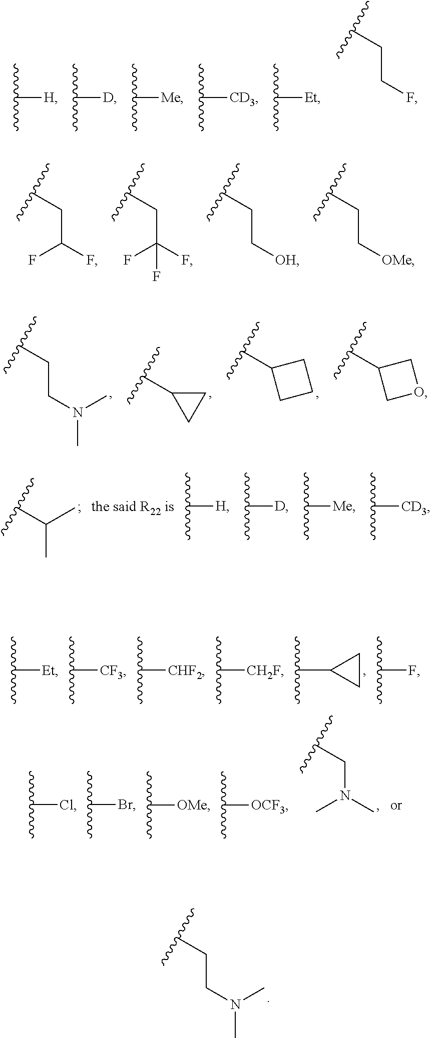

52. The compound or pharmaceutically acceptable salt thereof of any one of claim 48-51, wherein, the said R₂₁ is selected from 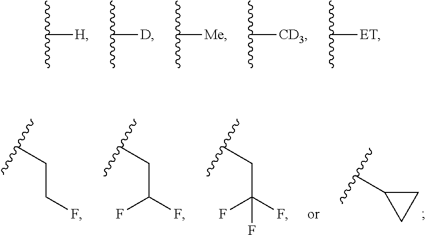
the said R₂₂ is

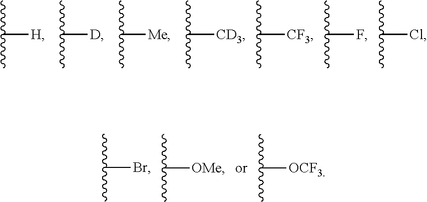

53. The compound or pharmaceutically acceptable salt thereof of claim 48, wherein, R₂₁ and R₂₂ are taken together with their intervening atoms to form a 5-membered heterocyclic ring, 6-membered heterocyclic ring, 7-membered heterocyclic ring, 5-membered carbocyclic ring, 6-membered carbocyclic ring, or 7-membered carbocyclic ring.

54. The compound or pharmaceutically acceptable salt thereof of claim 53, wherein, the said heterocyclic ring additionally have also one heteroatom selected from oxygen atom, nitrogen atom, or sulfur atom.

55. The compound or pharmaceutically acceptable salt thereof of claim 53 or 54, wherein, the said heterocyclic ring is 6-membered heterocyclic ring additionally having one oxygen atom, and the said carbocyclic ring is 6-membered carbocyclic ring.

56. The compound or pharmaceutically acceptable salt thereof of any one of claim 48-55, wherein, each of R₂₅ is optionally independently H, D, F, Cl, Br, CN, NH₂, OH, methyl, ethyl, propyl, i-propyl, —S-methyl, —S-ethyl, —S-propyl, methoxy, ethoxy, or propoxy; each of which is optionally unsubstituted or substituted with 1, 2, 3 instances of D, F, Cl, or Br.

57. The compound or pharmaceutically acceptable salt thereof of any one of claim 48-56, wherein, each of R₂₅ is optionally independently H, D, F, Cl, methyl, ethyl, —S-methyl, —S-ethyl, methoxy, or ethoxy; each of which is optionally unsubstituted or substituted with 1, 2, 3 instances of D or F.

58. The compound or pharmaceutically acceptable salt thereof of any one of claim 48-57, wherein, each of R₂₅ is optionally independently H, D, F, Cl, methyl, ethyl, —S-methyl, methoxy, or ethoxy; each of which is optionally unsubstituted or substituted with 1, 2, 3 instances of F.

59. The compound or pharmaceutically acceptable salt thereof of any one of claim 48-58, wherein, the said each of X or L is optionally independently selected from a covalent bond, —O—, —CO—, —NH—, —CH₂—, —CONH—, or —NHCO—.

60. The compound or pharmaceutically acceptable salt thereof of any one of claim 48-59, wherein, the said each of X is optionally independently selected from —NH—; the said each of L is optionally independently selected from —O—, —NH—, —CONH—, or —NHCO—.

61. The compound or pharmaceutically acceptable salt thereof of any one of claim 48-60, wherein, the said [symbol] is a bivalent bond and the said each of R₂₃ or R₂₄ is independently optionally selected from H, D, F, Cl, Br, —N(R″)₂, —CH₂N(R″)₂, —(CH₂)₂N(R″)₂, —(CH₂)₃N(R″)₂, or R″; each of which is optionally unsubstituted or substituted with 1, 2, or 3 instances of D, F, Cl, or Br.

62. The compound or pharmaceutically acceptable salt thereof of any one of claim 48-61, wherein, the said [symbol] is a bivalent bond and the said each of R₂₃ or R₂₄ is independently optionally selected from H, D, F, Cl, methyl, ethyl, propyl, i-propy, or cyclopropyl; each of which is optionally unsubstituted or substituted with 1, 2, or 3 instances of D, F or Cl.

63. The compound or pharmaceutically acceptable salt thereof of any one of claim 48-62, wherein, the said [symbol] is a bivalent bond and the said each of R₂₃ or R₂₄ is independently optionally selected from H, D, F, or methyl, and the methyl is optionally unsubstituted or substituted with 1, 2, or 3 instances of D or F.

64. The compound or pharmaceutically acceptable salt thereof of any one of claim 48-63, wherein, the said [symbol] is a trivalent bond and the said each of R₂₃ is absent, and the said each of R₂₄ is independently optionally selected from H, D, methyl, ethyl, propyl, iso-propyl, or cyclopropyl; each of which is optionally unsubstituted or substituted with 1, 2, or 3 instances of D, F, or Cl.

65. The compound or pharmaceutically acceptable salt thereof of any one of claim 48-64 or claim 90, wherein, the said [symbol] is a trivalent and the said each of R₂₃ is absent, and the said each of R₂₄ is independently optionally selected from H, D, or methyl; the said methyl is optionally unsubstituted or substituted with 1, 2, or 3 instances of D or F

66. The compound or pharmaceutically acceptable salt thereof of any one of claim 48-65, wherein, the said W is optionally independently selected from methyl; phenyl; 4-, 5-, or 6-membered saturated heterocyclic ring having 1 or 2 heteroatoms selected from N, O, or S; 5- or 6-membered heteroaryl having 1 or 2 heteroatoms selected from N or S; or 3-, 4-, 5-, or 6-membered saturated carbocyclic ring.

67. The compound or pharmaceutically acceptable salt thereof of any one of claim 48-66, wherein, the said W is optionally independently selected from 5- or 6-membered saturated heterocyclic ring having 1 nitrogen atom, 5-membered heteroaryl having 1 nitrogen atom and optionally additionally 1 sulfur atom, 6-membered heteroaryl having 1 or 2 nitrogen atom, 3-membered saturated carbocyclic ring, 4-membered saturated carbocyclic ring, 5-membered saturated carbocyclic ring, or 6-membered saturated carbocyclic ring;

68. The compound or pharmaceutically acceptable salt thereof of any one of claim 48-67, wherein, the said each of R_{w} is optionally independently selected from D, F, Cl, Br, OH, OMe, CN, NH₂, methyl, ethyl, propyl, i-propyl, methyl substituted with F or Cl, ethyl substituted with F or Cl, propyl substituted with F or Cl, i-propyl substituted with F or Cl, cyclopropyl, cyclobutyl, cyclopentyl, viny or ethynyl; and each of which is optionally independently unsubstituted or substituted with 1, 2, or 3 instances of R.

69. The compound or pharmaceutically acceptable salt thereof of any one of claim 48-68, wherein, the said each of R_{w} is optionally independently selected from D; F; Cl; methyl; methyl substituted with 1, 2, or 3 instance of F or D; methoxy; or cyclopropyl.

70. The compound or pharmaceutically acceptable salt thereof of any one of claim 48-69, wherein, the said each of R_{z} is optionally independently selected from D, F, Cl, Br, CN, —N(R″)₂, —OR″, or R″.

71. The compound or pharmaceutically acceptable salt thereof of any one of claim 48-70, wherein, the said each of R_{z} is optionally independently selected from D, F, Cl, Br, CN, NH₂, methyl, ethyl, propyl, or i-propyl.

72. The compound or pharmaceutically acceptable salt thereof of any one of claim 48-71, wherein, the said each of R is optionally independently selected from D, F, Cl, or methyl.

73. The compound or pharmaceutically acceptable salt thereof of any one of claim 48-71, wherein, the said each of R″ is optionally independently methyl, ethyl, propyl, i-propyl, cyclopropyl, cyclobutyl, or cyclopentyl; each of which is optionally unsubstituted or substituted with 1, 2, or 3 instances of F, Cl, or D.

74. The compound or pharmaceutically acceptable salt thereof of any one of claim 48-71, wherein, the said each of R″ is optionally independently methyl, ethyl, or cyclopropyl; each of which is optionally unsubstituted or substituted with 1, 2, or 3 instances of F or D.

75. The compound is independently selected from:
N-(4-(3-(3-methoxy-4-((4-methylpyrimidin-2-yl)oxy)phenyl)imidazo[1,2-b]pyridazin-2-yl)phenyl)acrylamide;
N-(4-(9-(3-methoxy-4-((4-methylpyrimidin-2-yl)oxy)phenyl)-9H-purin-8-yl)phenyl) acrylamide;
N-(4-(7-(3-methoxy-4-((4-methylpyrimidin-2-yl)oxy)phenyl)imidazo[1,2-b][1,2,4]triazin-6-yl)phenyl)acrylamide;
N-(4-(4-amino-3-(3-methoxy-4-((4-methylpyrimidin-2-yl)oxy)phenyl)-1H-pyrazolo[3,4-d]pyrimidin-1-yl)phenyl)acrylamide;
N-(4-(5-amino-3-(3-methoxy-4-((4-methylpyrimidin-2-yl)oxy)phenyl)imidazo[1,2-a]pyrimidin-2-yl)phenyl)acrylamide;
N-(4-(4-amino-5-cyano-3-(3-methoxy-4-((4-methylpyrimidin-2-yl)oxy)phenyl)-1-methyl-1H-pyrrolo[2,3-b]pyridin-2-yl)phenyl)acrylamide;
N-(4-(4-amino-7-cyano-3-(3-methoxy-4-((4-methylpyrimidin-2-yl)oxy)phenyl)-1-methyl-1H-pyrrolo[3,2-c]pyridin-2-yl)phenyl)acrylamide;
N-(4-(4-amino-5-(3-methoxy-4-((4-methylpyrimidin-2-yl)oxy)phenyl)-2,7-dimethyl-7H-pyrrolo[2,3-d]pyrimidin-6-yl)phenyl)acrylamide;
N-(4-(4-amino-2-chloro-5-(3-methoxy-4-((4-methylpyrimidin-2-yl)oxy)phenyl)-7-methyl-7H-pyrrolo[2,3-d]pyrimidin-6-yl)phenyl)acrylamide;
N-(4-(4-methoxy-5-(3-methoxy-4-((4-methylpyrimidin-2-yl)oxy)phenyl)-7-methyl-7H-pyrrolo[2,3-d]pyrimidin-6-yl)phenyl)acrylamide;
N-(4-(4-cyclopropyl-5-(3-methoxy-4-((4-methylpyrimidin-2-yl)oxy)phenyl)-7-methyl-7H-pyrrolo[2,3-d]pyrimidin-6-yl)phenyl)acrylamide;
N-(4-(4-(difluoromethoxy)-5-(3-methoxy-4-((4-methylpyrimidin-2-yl)oxy)phenyl)-7-methyl-7H-pyrrolo[2,3-d]pyrimidin-6-yl)phenyl)acrylamide;
N-(4-(4-cyano-5-(3-methoxy-4-((4-methylpyrimidin-2-yl)oxy)phenyl)-7-methyl-7H-pyrrolo[2,3-d]pyrimidin-6-yl)phenyl)acrylamide;
N-(4-(4-amino-6-(3-methoxy-4-((4-methylpyrimidin-2-yl)oxy)phenyl)-7-methyl-7H-pyrrolo[2,3-d]pyrimidin-5-yl)phenyl)acrylamide;
N-(4-(4-amino-5-(4-(pyrrolidine-1-carbonyl)phenyl)pyrazolo[5,1-f][1,2,4]triazin-6-yl)-3-fluorophenyl)methacrylamide;
N-(4-(4-amino-5-(3-methoxy-4-((5-(methylamino)pyrimidin-2-yl)oxy)phenyl)pyrazolo[5,1-f][1,2,4]triazin-6-yl)phenyl)acrylamide;
N-(4-(4-amino-5-(3-methoxy-4-(pyrrolidine-1-carbonyl)phenyl)pyrazolo[5,1-f][1,2,4]triazin-6-yl)phenyl)acrylamide;
N-(4-(4-amino-5-(3-methoxy-4-((1-methyl-1H-pyrazol-3-yl)oxy)phenyl)pyrazolo[5,1-f][1,2,4]triazin-6-yl)phenyl)acrylamide;
N-(4-(4-amino-5-(4-(pyrimidin-2-yloxy)phenyl)pyrazolo[5,1-f][1,2,4]triazin-6-yl)phenyl) acrylamide;
2-(4-acrylamidophenyl)-4-amino-3-(3-methoxy-4-((4-methylpyrimidin-2-yl)oxy)phenyl)-1-methyl-1H-pyrrolo[3,2-c]pyridine-7-carboxamide;
N-(4-(4-amino-7-cyano-3-(3-methoxy-4-((4-methylpyrimidin-2-yl)oxy)phenyl)-1H-pyrrolo[3,2-c]pyridin-2-yl)phenyl)acrylamide;
4-(2-(4-acrylamidophenyl)-4-amino-7-cyano-1H-pyrrolo[3,2-c]pyridin-3-yl)-N-cyclopropyl-2-methoxybenzamide;
4-(6-(4-acrylamidophenyl)-4-aminopyrazolo[5,1-f][1,2,4]triazin-5-yl)-2-(difluoromethoxy)-N-((1r,3r)-3-fluorocyclobutyl)benzamide;
(S)-4-(6-(4-acrylamidophenyl)-4-aminopyrazolo[5,1-f][1,2,4]triazin-5-yl)-2-fluoro-N-(tetrahydrofuran-3-yl)benzamide;
4-(6-(4-acrylamidophenyl)-4-aminopyrazolo[5,1-f][1,2,4]triazin-5-yl)-N-cyclobutyl-2-methoxybenzamide;
4-(4-amino-6-(4-methacrylamidophenyl)pyrazolo[5,1-f][1,2,4]triazin-5-yl)-N-isobutylbenzamide;
N-(4-(4-amino-5-(3-methoxy-4-(pyrimidin-2-yloxy)phenyl)pyrazolo[5,1-f][1,2,4]triazin-6-yl)phenyl)acrylamide;
N-(4-(4-amino-5-(3-fluoro-4-(pyrimidin-2-yloxy)phenyl)pyrazolo[5,1-f][1,2,4]triazin-6-yl)phenyl)acrylamide;
N-(4-(4-amino-5-(4-((4-methylpyrimidin-2-yl)oxy)phenyl)pyrazolo[5,1-f][1,2,4]triazin-6-yl)phenyl)acrylamide;
N-(4-(4-amino-5-(4-phenoxyphenyl)pyrazolo[5,1-f][1,2,4]triazin-6-yl)phenyl) acrylamide;
N-(4-(4-amino-5-(4-(pyridin-2-yloxy)phenyl)pyrazolo[5,1-f][1,2,4]triazin-6-yl)phenyl)acrylamide;
N-(4-(4-amino-5-(4-(cyclopentyloxy)phenyl)pyrazolo[5,1-f][1,2,4]triazin-6-yl)phenyl)acrylamide;
N-(4-(4-amino-5-(4-(pyrimidin-2-yloxy)phenyl)pyrazolo[5,1-f][1,2,4]triazin-6-yl)-2-fluorophenyl)acrylamide;
N-(4-(4-amino-5-(4-(pyrimidin-2-yloxy)phenyl)pyrazolo[5,1-f][1,2,4]triazin-6-yl)-3-fluorophenyl)acrylamide;
4-(2-(4-acrylamidophenyl)-4-amino-7-cyano-1-(2-hydroxyethyl)-1H-pyrrolo[3,2-c]pyridin-3-yl)-N-cyclobutyl-2-methoxybenzamide;
4-(6-(4-acrylamidophenyl)-4-aminopyrazolo[5,1-f][1,2,4]triazin-5-yl)-N-isobutylbenzamide;
4-(6-(4-acrylamidophenyl)-4-aminopyrazolo[5,1-f][1,2,4]triazin-5-yl)-N-cyclobutylbenzamide;
4-(6-(4-acrylamidophenyl)-4-aminopyrazolo[5,1-f][1,2,4]triazin-5-yl)-N-cyclobutyl-2-fluorobenzamide;
methyl 4-(6-(4-acrylamidophenyl)-4-aminopyrazolo[5,1-f][1,2,4]triazin-5-yl)-2-methoxybenzoate;
4-(6-(4-acrylamidophenyl)-4-aminopyrazolo[5,1-f][1,2,4]triazin-5-yl)-2-methoxy-N-(2,2,2-trifluoroethyl)benzamide;
4-(6-(4-acrylamidophenyl)-4-aminopyrazolo[5,1-f][1,2,4]triazin-5-yl)-2-methoxy-N-(oxetan-3-yl)benzamide;
4-(6-(4-acrylamidophenyl)-4-aminopyrazolo[5,1-f][1,2,4]triazin-5-yl)-2-methoxy-N-((1-methyl-1H-pyrazol-3-yl)methyl)benzamide;
4-(6-(4-acrylamidophenyl)-4-aminopyrazolo[5,1-f][1,2,4]triazin-5-yl)-N-(cyclobutylmethyl)-2-methoxybenzamide;
4-(6-(4-acrylamidophenyl)-4-aminopyrazolo[5,1-f][1,2,4]triazin-5-yl)-N-((1s,3s)-3-hydroxycyclobutyl)-2-methoxybenzamide;
4-(6-(4-acrylamidophenyl)-4-aminopyrazolo[5,1-f][1,2,4]triazin-5-yl)-N-((1-cyanocyclopropyl)methyl)-2-methoxybenzamide;
4-(4-amino-6-(4-(2-fluoroacrylamido)phenyl)pyrazolo[5,1-f][1,2,4]triazin-5-yl)-N-isobutylbenzamide;
4-(6-(4-acrylamidophenyl)-4-aminopyrazolo[5,1-f][1,2,4]triazin-5-yl)-N-cyclobutyl-2-methoxy-N-methylbenzamide;
4-(6-(4-acrylamidophenyl)-4-aminopyrazolo[5,1-f][1,2,4]triazin-5-yl)-N-cyclobutylcyclohex-3-ene-1-carboxamide;
4-(6-(4-acrylamido-3-fluorophenyl)-4-aminopyrazolo[5,1-f][1,2,4]triazin-5-yl)-N-isobutylbenzamide;
4-(6-(4-acrylamido-2-fluorophenyl)-4-aminopyrazolo[5,1-f][1,2,4]triazin-5-yl)-N-isobutylbenzamide;
4-(6-(4-acrylamido-3-methoxyphenyl)-4-aminopyrazolo[5,1-f][1,2,4]triazin-5-yl)-N-isobutylbenzamide;
4-(6-(4-acrylamido-2-methoxyphenyl)-4-aminopyrazolo[5,1-f][1,2,4]triazin-5-yl)-N-isobutylbenzamide;
4-(6-(4-acrylamidophenyl)-4-aminopyrazolo[5,1-f][1,2,4]triazin-5-yl)-N-cyclopentyl-2-methoxybenzamide;
4-(6-(4-acrylamidophenyl)-4-aminopyrazolo[5,1-f][1,2,4]triazin-5-yl)-2-methoxy-N-(2-methoxy-2-methylpropyl)benzamide;
4-(6-(4-acrylamidophenyl)-4-aminopyrazolo[5,1-f][1,2,4]triazin-5-yl)-N-(2-hydroxy-2-methylpropyl)-2-methoxybenzamide;
4-(6-(4-acrylamidophenyl)-4-aminopyrazolo[5,1-f][1,2,4]triazin-5-yl)-N-cyclohexyl-2-methoxybenzamide;
4-(2-(4-acrylamidophenyl)-4-amino-7-cyano-1-(2-methoxyethyl)-1H-pyrrolo[3,2-c]pyridin-3-yl)-N-cyclobutyl-2-methoxybenzamide;
4-(6-(4-acrylamidophenyl)-4-aminopyrazolo[5,1-f][1,2,4]triazin-5-yl)-N-ethyl-2-methoxybenzamide;
4-(6-(4-acrylamidophenyl)-4-aminopyrazolo[5,1-f][1,2,4]triazin-5-yl)-N-isopropyl-2-methoxybenzamide;
4-(6-(4-acrylamidophenyl)-4-aminopyrazolo[5,1-f][1,2,4]triazin-5-yl)-N-(2-(dimethylamino)ethyl)-2-methoxybenzamide;
4-(6-(4-acrylamidophenyl)-4-aminopyrazolo[5,1-f][1,2,4]triazin-5-yl)-2-methoxy-N-((1s,3s)-3-methoxycyclobutyl)benzamide;
4-(2-(4-acrylamidophenyl)-4-amino-7-cyano-1-methyl-1H-pyrrolo[3,2-c]pyridin-3-yl)-N-isobutyl-2-methoxybenzamide;
4-(2-(4-acrylamidophenyl)-4-amino-7-cyano-1-methyl-1H-pyrrolo[3,2-c]pyridin-3-yl)-N-cyclobutylbenzamide;
4-(2-(4-acrylamidophenyl)-4-amino-7-cyano-1-methyl-1H-pyrrolo[3,2-c]pyridin-3-yl)-N-cyclobutyl-2-methoxybenzamide;
N-(4-(4-amino-7-cyano-1-methyl-3-(4-(pyrimidin-2-yloxy)phenyl)-1H-pyrrolo[3,2-c]pyridin-2-yl)phenyl)acrylamide;
N-(4-(4-amino-7-cyano-3-(3-methoxy-4-(pyrimidin-2-yloxy)phenyl)-1-methyl-1H-pyrrolo[3,2-c]pyridin-2-yl)phenyl)acrylamide;
N-(4-(4-amino-7-cyano-1-methyl-3-(4-((4-methylpyrimidin-2-yl)oxy)phenyl)-1H-pyrrolo[3,2-c]pyridin-2-yl)phenyl)acrylamide;
4-(4-amino-6-(4-methacrylamidophenyl)pyrazolo[5,1-f][1,2,4]triazin-5-yl)-2-methoxy-N-(2,2,2-trifluoroethyl)benzamide;
(E)-4-(4-amino-6-(4-(4-(dimethylamino)but-2-enamido)phenyl)pyrazolo[5,1-f][1,2,4]triazin-5-yl)-2-methoxy-N-(2,2,2-trifluoroethyl)benzamide;
4-(6-(4-acrylamidophenyl)-4-aminopyrazolo[5,1-f][1,2,4]triazin-5-yl)-N-cyclobutyl-2-(trifluoromethoxy)benzamide;
4-(6-(4-acrylamidophenyl)-4-aminopyrazolo[5,1-f][1,2,4]triazin-5-yl)-N-cyclobutyl-2-methylbenzamide;
4-(6-(4-acrylamidophenyl)-4-aminopyrazolo[5,1-f][1,2,4]triazin-5-yl)-N-cyclobutyl-2-ethoxybenzamide;
4-(6-(4-acrylamidophenyl)-4-aminopyrazolo[5,1-f][1,2,4]triazin-5-yl)-N-cyclobutyl-2-methoxy-5-methylbenzamide;
4-(4-amino-6-(4-(2-(trifluoromethyl)acrylamido)phenyl)pyrazolo[5,1-f][1,2,4]triazin-5-yl)-2-methoxy-N-(2,2,2-trifluoroethyl)benzamide;
4-(4-amino-6-(4-(N-methylacrylamido)phenyl)pyrazolo[5,1-f][1,2,4]triazin-5-yl)-2-methoxy-N-(2,2,2-trifluoroethyl)benzamide;
4-(6-(4-acrylamidophenyl)-4-aminopyrazolo[5,1-f][1,2,4]triazin-5-yl)-2-methoxy-N-methyl-N-(2,2,2-trifluoroethyl)benzamide;
N-(4-(4-amino-5-(3-methoxy-4-(4-(trifluoromethyl)oxazol-2-yl)phenyl)pyrazolo[5,1-f][1,2,4]triazin-6-yl)phenyl)acrylamide;
4-(6-(4-acrylamidophenyl)-4-aminopyrazolo[5,1-f][1,2,4]triazin-5-yl)-N-isobutyl-2-methoxybenzamide;
N-(4-(4-amino-5-(2,2-dimethyl-4-oxo-3,4-dihydro-2H-benzo[e][1,3]oxazin-7-yl)pyrazolo[5,1-f][1,2,4]triazin-6-yl)phenyl)acrylamide;
N-(4-(4-amino-5-(5-oxo-2,3,4,5-tetrahydrobenzo[f][1,4]oxazepin-8-yl)pyrazolo[5,1-f][1,2,4]triazin-6-yl)phenyl)acrylamide;
4-(6-(4-acrylamidophenyl)-4-aminopyrazolo[5,1-f][1,2,4]triazin-5-yl)-N-cyclobutyl-2-methoxy-6-methylbenzamide;
4-(6-(4-acrylamido-2-methoxyphenyl)-4-aminopyrazolo[5,1-f][1,2,4]triazin-5-yl)-2-methoxy-N-(2,2,2-trifluoroethyl)benzamide;
4-(6-(4-acrylamido-2-fluorophenyl)-4-aminopyrazolo[5,1-f][1,2,4]triazin-5-yl)-2-methoxy-N-(2,2,2-trifluoroethyl)benzamide;
4-(6-(4-acrylamido-2-methylphenyl)-4-aminopyrazolo[5,1-f][1,2,4]triazin-5-yl)-2-methoxy-N-(2,2,2-trifluoroethyl)benzamide;
4-(6-(2-acryloyl-2-azaspiro[3.3]heptan-6-yl)-4-aminopyrazolo[5,1-f][1,2,4]triazin-5-yl)-2-methoxy-N-(2,2,2-trifluoroethyl)benzamide;
4-(6-(4-acrylamidophenyl)-4-aminopyrazolo[5,1-f][1,2,4]triazin-5-yl)-N-cyclopropyl-2-methoxybenzamide;
4-(6-(6-acrylamidopyridin-3-yl)-4-aminopyrazolo[5,1-f][1,2,4]triazin-5-yl)-2-methoxy-N-(2,2,2-trifluoroethyl)benzamide;
4-(6-(4-acrylamidophenyl)-4-aminopyrazolo[5,1-f][1,2,4]triazin-5-yl)-2-(difluoromethoxy)-N-(2,2,2-trifluoroethyl)benzamide;
4-(4-amino-6-(4-(2-fluoroacrylamido)phenyl)pyrazolo[5,1-f][1,2,4]triazin-5-yl)-2-methoxy-N-methyl-N-(2,2,2-trifluoroethyl)benzamide;
4-(4-amino-6-(4-(2-fluoroacrylamido)phenyl)pyrazolo[5,1-f][1,2,4]triazin-5-yl)-N-cyclopentyl-2-methoxybenzamide;
4-(4-amino-6-(4-(2-fluoroacrylamido)phenyl)pyrazolo[5,1-f][1,2,4]triazin-5-yl)-N-ethyl-2-methoxybenzamide;
4-(4-amino-6-(4-(2-fluoroacrylamido)phenyl)pyrazolo[5,1-f][1,2,4]triazin-5-yl)-N-cyclobutyl-2-methoxybenzamide;
4-(4-amino-7-cyano-2-(4-(2-fluoroacrylamido)phenyl)-1-methyl-1H-pyrrolo[3,2-c]pyridin-3-yl)-N-cyclobutylbenzamide;
4-(6-(4-acrylamidophenyl)-4-aminopyrazolo[5,1-f][1,2,4]triazin-5-yl)-N-(3,3-difluorocyclobutyl)-2-methoxybenzamide;
4-(4-amino-6-(4-(2-fluoroacrylamido)phenyl)pyrazolo[5,1-f][1,2,4]triazin-5-yl)-N-(3,3-difluorocyclobutyl)-2-methoxybenzamide;
4-(4-amino-6-(4-(2-fluoroacrylamido)-2-methylphenyl)pyrazolo[5,1-f][1,2,4]triazin-5-yl)-2-methoxy-N-(2,2,2-trifluoroethyl)benzamide;
4-(4-amino-6-(4-(2-fluoroacrylamido)-2-methoxyphenyl)pyrazolo[5,1-f][1,2,4]triazin-5-yl)-2-methoxy-N-(2,2,2-trifluoroethyl)benzamide;
4-(4-amino-6-(4-(2-fluoroacrylamido)phenyl)pyrazolo[5,1-f][1,2,4]triazin-5-yl)-N-cyclopropyl-2-methoxybenzamide;
4-(4-amino-7-cyano-2-(4-(2-fluoroacrylamido)phenyl)-1-methyl-1H-pyrrolo[3,2-c]pyridin-3-yl)-N-cyclopropyl-2-methoxybenzamide;
4-(2-(4-acrylamidophenyl)-4-amino-7-cyano-1-methyl-1H-pyrrolo[3,2-c]pyridin-3-yl)-N-cyclopropyl-2-methoxybenzamide;
4-(2-(4-acrylamidophenyl)-4-amino-7-cyano-1-methyl-1H-pyrrolo[3,2-c]pyridin-3-yl)-N-(2,2,2-trifluoroethyl)benzamide;
4-(2-(4-acrylamidophenyl)-4-amino-7-cyano-1-methyl-1H-pyrrolo[3,2-c]pyridin-3-yl)-N-ethyl-2-methoxybenzamide;
4-(2-(4-acrylamidophenyl)-4-amino-7-cyano-1-methyl-1H-pyrrolo[3,2-c]pyridin-3-yl)-N-isopropyl-2-methoxybenzamide;
4-(2-(4-acrylamidophenyl)-4-amino-7-cyano-1-methyl-1H-pyrrolo[3,2-c]pyridin-3-yl)-N-cyclopentyl-2-methoxybenzamide;
4-(2-(4-acrylamidophenyl)-4-amino-7-cyano-1-methyl-1H-pyrrolo[3,2-c]pyridin-3-yl)-N-cyclohexyl-2-methoxybenzamide;
4-(2-(4-acrylamidophenyl)-4-amino-7-cyano-1-methyl-1H-pyrrolo[3,2-c]pyridin-3-yl)-N-((1-cyanocyclopropyl)methyl)-2-methoxybenzamide;
4-(2-(4-acrylamido-2-methoxyphenyl)-4-amino-7-cyano-1-methyl-1H-pyrrolo[3,2-c]pyridin-3-yl)-2-methoxy-N-(2,2,2-trifluoroethyl)benzamide;
5-(4-acrylamidophenyl)-4-(3-methoxy-4-((6-methylpyridin-2-yl)oxy)phenyl)nicotinamide;
5-(4-acrylamidophenyl)-2-amino-4-(3-methoxy-4-((6-methylpyridin-2-yl)oxy)phenyl)-6-methylnicotinamide;
N-(4-(4-amino-3-(3-methoxy-4-((6-methylpyridin-2-yl)oxy)phenyl)-7-oxo-6,7-dihydro-2H-pyrazolo[3,4-d]pyridazin-2-yl)phenyl)acrylamide;
N-(4-(4-amino-3-(3-methoxy-4-((6-methylpyridin-2-yl)oxy)phenyl)-7-oxo-6,7-dihydro-2H-pyrazolo[3,4-d]pyridazin-2-yl)phenyl)methacrylamide;
1-(3-((4-amino-5-(3-methoxy-4-((6-methylpyridin-2-yl)oxy)phenyl)-7H-pyrrolo[2,3-d]pyrimidin-7-yl)methyl)piperidin-1-yl)prop-2-en-1-one;
N-(4-(4-amino-3-(3-methoxy-4-((6-methylpyridin-2-yl)oxy)phenyl)-7-oxo-6,7-dihydro-2H-pyrazolo[3,4-d]pyridazin-2-yl)-2-fluorophenyl)acrylamide;
5-(4-acrylamidophenyl)-2-amino-4-(3-methoxy-4-((6-methylpyridin-2-yl)oxy)phenyl)-6-methylnicotinamide;
4-(4-amino-2-(4-methacrylamidophenyl)-7-oxo-6,7-dihydro-2H-pyrazolo[3,4-d]pyridazin-3-yl)-N-isobutylbenzamide;
4-(2-(4-acrylamidophenyl)-4-amino-7-oxo-6,7-dihydro-2H-pyrazolo[3,4-d]pyridazin-3-yl)-N-isobutylbenzamide;
N-(4-(4-cyano-6-(3-methoxy-4-((6-methylpyridin-2-yl)oxy)phenyl)-7-methyl-7H-pyrrolo[2,3-d]pyrimidin-5-yl)phenyl)acrylamide;
N-(4-(4-chloro-6-(3-methoxy-4-((6-methylpyridin-2-yl)oxy)phenyl)-7-methyl-7H-pyrrolo[2,3-d]pyrimidin-5-yl)phenyl)acrylamide;
N-(4-(4-methoxy-6-(3-methoxy-4-((6-methylpyridin-2-yl)oxy)phenyl)-7-methyl-7H-pyrrolo[2,3-d]pyrimidin-5-yl)phenyl)acrylamide;
4-(2-(4-acrylamidophenyl)-4-amino-7-oxo-6,7-dihydro-2H-pyrazolo[3,4-d]pyridazin-3-yl)-N-cyclobutylbenzamide;
N-(4-(4-amino-7-oxo-3-(4-(pyrrolidine-1-carbonyl)phenyl)-6,7-dihydro-2H-pyrazolo[3,4-d]pyridazin-2-yl)phenyl)acrylamide;
4-(2-(4-acrylamidophenyl)-4-amino-7-oxo-6,7-dihydro-2H-pyrazolo[3,4-d]pyridazin-3-yl)-N-(oxetan-3-yl)benzamide;
N-(4-(4-amino-3-(3-methoxy-4-((6-methylpyridin-2-yl)oxy)phenyl)-7-oxo-6,7-dihydro-2H-pyrazolo[3,4-d]pyridazin-2-yl)phenyl)-2-fluoroacrylamide;
N-(4-(4-amino-3-(4-(cyclopentyloxy)phenyl)-7-oxo-6,7-dihydro-2H-pyrazolo[3,4-d]pyridazin-2-yl)phenyl)acrylamide;
4-(2-(4-acrylamidophenyl)-4-amino-7-oxo-6,7-dihydro-2H-pyrazolo[3,4-d]pyridazin-3-yl)-N-(pyrrolidin-3-yl)benzamide;
4-(2-(4-acrylamidophenyl)-4-amino-7-oxo-6,7-dihydro-2H-pyrazolo[3,4-d]pyridazin-3-yl)-N-(tetrahydro-2H-pyran-3-yl)benzamide;
4-(2-(4-acrylamidophenyl)-4-amino-7-oxo-6,7-dihydro-2H-pyrazolo[3,4-d]pyridazin-3-yl)-N-cyclobutyl-2-methoxybenzamide;
N-(4-(4-amino-3-(3-methoxy-4-((4-methylpyrimidin-2-yl)oxy)phenyl)-7-oxo-6,7-dihydro-2H-pyrazolo[3,4-d]pyridazin-2-yl)phenyl)methacrylamide;
N-(4-(4-amino-3-(3-methoxy-4-((4-methylpyrimidin-2-yl)oxy)phenyl)-7-oxo-6,7-dihydro-2H-pyrazolo[3,4-d]pyridazin-2-yl)phenyl)acrylamide;
4-(2-(4-acrylamidophenyl)-4-amino-7-oxo-6,7-dihydro-2H-pyrazolo[3,4-d]pyridazin-3-yl)-N-isobutyl-2-methoxybenzamide;
N-(4-(4-amino-3-(4-((4-chloropyridin-2-yl)oxy)-3-methoxyphenyl)-7-oxo-6,7-dihydro-2H-pyrazolo[3,4-d]pyridazin-2-yl)phenyl)acrylamide;
4-(4-amino-2-(4-(2-fluoroacrylamido)phenyl)-7-oxo-6,7-dihydro-2H-pyrazolo[3,4-d]pyridazin-3-yl)-N-cyclobutyl-2-methoxybenzamide;
N-(4-(4-amino-3-(4-(cyclopentyloxy)-3-methoxyphenyl)-7-oxo-6,7-dihydro-2H-pyrazolo[3,4-d]pyridazin-2-yl)phenyl)acrylamide;
N-(4-(4-amino-3-(4-cyclobutoxyphenyl)-7-oxo-6,7-dihydro-2H-pyrazolo[3,4-d]pyridazin-2-yl)phenyl)acrylamide;
N-(4-(4-amino-3-(4-(cyclohexyloxy)phenyl)-7-oxo-6,7-dihydro-2H-pyrazolo[3,4-d]pyridazin-2-yl)phenyl)acrylamide;
N-(4-(4-amino-3-(4-isobutoxyphenyl)-7-oxo-6,7-dihydro-2H-pyrazolo[3,4-d]pyridazin-2-yl)phenyl)acrylamide;
N-(4-(4-amino-3-(4-(cyclopentyloxy)-3-fluorophenyl)-7-oxo-6,7-dihydro-2H-pyrazolo[3,4-d]pyridazin-2-yl)phenyl)acrylamide;
5-(4-acrylamidophenyl)-6-(3-methoxy-4-((6-methylpyridin-2-yl)oxy)phenyl)-1H-indazole-7-carboxamide;
N-(4-(4-amino-7-oxo-3-(4-((tetrahydro-2H-pyran-4-yl)oxy)phenyl)-6,7-dihydro-2H-pyrazolo[3,4-d]pyridazin-2-yl)phenyl)acrylamide;
N-(4-(4-amino-3-(6-(cyclohexyloxy)pyridin-3-yl)-7-oxo-6,7-dihydro-2H-pyrazolo[3,4-d]pyridazin-2-yl)phenyl)acrylamide;
N-(4-(4-amino-3-(4-(cyclohexyl(methyl)amino)phenyl)-7-oxo-6,7-dihydro-2H-pyrazolo[3,4-d]pyridazin-2-yl)phenyl)acrylamide;
N-(4-(4-amino-3-(4-((4,4-difluorocyclohexyl)oxy)phenyl)-7-oxo-6,7-dihydro-2H-pyrazolo[3,4-d]pyridazin-2-yl)phenyl)acrylamide;
N-(4-(4-amino-3-(4-(cyclohexylthio)phenyl)-7-oxo-6,7-dihydro-2H-pyrazolo[3,4-d]pyridazin-2-yl)phenyl)acrylamide;
N-(4-(4-amino-3-(4-(cyclopentyloxy)phenyl)-7-oxo-6,7-dihydro-2H-pyrazolo[3,4-d]pyridazin-2-yl)phenyl)methacrylamide;
4-(2-(4-acrylamido-2-fluorophenyl)-4-amino-7-cyano-1-methyl-1H-pyrrolo[3,2-c]pyridin-3-yl)-2-methoxy-N-(2,2,2-trifluoroethyl)benzamide;
4-(4-amino-7-cyano-2-(2-fluoro-4-(2-fluoroacrylamido)phenyl)-1-methyl-1H-pyrrolo[3,2-c]pyridin-3-yl)-2-methoxy-N-(2,2,2-trifluoroethyl)benzamide;
4-(4-amino-6-(4-(2-fluoroacrylamido)-2-methoxyphenyl)pyrazolo[5,1-f][1,2,4]triazin-5-yl)-N-cyclopropyl-2-methoxybenzamide;
4-(4-amino-6-(2-fluoro-4-(2-fluoroacrylamido)phenyl)pyrazolo[5,1-f][1,2,4]triazin-5-yl)-N-cyclopropyl-2-methoxybenzamide;
4-(6-(4-acrylamido-2-methoxyphenyl)-4-aminopyrazolo[5,1-f][1,2,4]triazin-5-yl)-N-cyclopropyl-2-methoxybenzamide;
4-(6-(4-acrylamido-2-fluorophenyl)-4-aminopyrazolo[5,1-f][1,2,4]triazin-5-yl)-N-cyclopropyl-2-methoxybenzamide;
4-(2-(4-acrylamido-2-methoxyphenyl)-4-amino-7-cyano-1-methyl-1H-pyrrolo[3,2-c]pyridin-3-yl)-N-cyclopropyl-2-methoxybenzamide;
4-(2-(4-acrylamido-2-fluorophenyl)-4-amino-7-cyano-1-methyl-1H-pyrrolo[3,2-c]pyridin-3-yl)-N-cyclopropyl-2-methoxybenzamide;
4-(4-amino-7-cyano-2-(4-(2-fluoroacrylamido)phenyl)-1-methyl-1H-pyrrolo[3,2-c]pyridin-3-yl)-N-isopropyl-2-methoxybenzamide;
(S)-4-(4-amino-6-(4-(2-fluoroacrylamido)phenyl)pyrazolo[5,1-f][1,2,4]triazin-5-yl)-N-(3,3-difluorocyclopentyl)-2-methoxybenzamide;
4-(2-(4-acrylamidophenyl)-4-amino-7-cyano-1-methyl-1H-pyrrolo[3,2-c]pyridin-3-yl)-2-fluoro-N-(2,2,2-trifluoroethyl)benzamide;
N-(4-(4-amino-7-cyano-3-(4-(cyclopentyloxy)phenyl)-1-methyl-1H-pyrrolo[3,2-c]pyridin-2-yl)phenyl)acrylamide;
4-(6-(4-acrylamidophenyl)-4-aminopyrazolo[5,1-f][1,2,4]triazin-5-yl)-2-methoxy-N-(1-methylcyclopropyl)benzamide;
4-(4-amino-6-(4-(2-fluoroacrylamido)phenyl)pyrazolo[5,1-f][1,2,4]triazin-5-yl)-N-(cyclopropylmethyl)-2-methoxybenzamide;
N-(4-(4-amino-5-(4-(cyclopentyl(methyl)amino)phenyl)pyrazolo[5,1-f][1,2,4]triazin-6-yl)phenyl)acrylamide;
N-(4-(4-amino-5-(4-cyclobutoxyphenyl)pyrazolo[5,1-f][1,2,4]triazin-6-yl)phenyl)acrylamide;
4-(4-amino-6-(6-ethynylpyridin-3-yl)pyrazolo[5,1-f][1,2,4]triazin-5-yl)-2-methoxy-N-(2,2,2-trifluoroethyl)benzamide;
4-(2-(4-acrylamidophenyl)-4-amino-7-cyano-1-methyl-1H-pyrrolo[3,2-c]pyridin-3-yl)-N-(2,2-difluorocyclopropyl)-2-methoxybenzamide;
4-(2-(4-acrylamidophenyl)-4-amino-7-cyano-1-methyl-1H-pyrrolo[3,2-c]pyridin-3-yl)-N-(3-fluorocyclobutyl)-2-methoxybenzamide;
4-(2-(4-acrylamidophenyl)-4-amino-7-cyano-1-methyl-1H-pyrrolo[3,2-c]pyridin-3-yl)-2-methoxy-N-(1-methylcyclopropyl)benzamide;
4-(2-(4-acrylamidophenyl)-4-amino-7-cyano-1-methyl-1H-pyrrolo[3,2-c]pyridin-3-yl)-N-(cyclopropylmethyl)-2-methoxybenzamide;
4-(4-amino-7-cyano-2-(4-(2-fluoroacrylamido)-2-methoxyphenyl)-1-methyl-1H-pyrrolo[3,2-c]pyridin-3-yl)-N-cyclopropyl-2-methoxybenzamide;
N-(4-(4-amino-3-(4-cyclopropoxyphenyl)-7-oxo-6,7-dihydro-2H-pyrazolo[3,4-d]pyridazin-2-yl)phenyl)acrylamide;
N-(4-(4-amino-3-(4-((3-fluorocyclopentyl)oxy)phenyl)-7-oxo-6,7-dihydro-2H-pyrazolo[3,4-d]pyridazin-2-yl)phenyl)acrylamide;
N-(4-(4-amino-3-(4-((3,3-difluorocyclopentyl)oxy)phenyl)-7-oxo-6,7-dihydro-2H-pyrazolo[3,4-d]pyridazin-2-yl)phenyl)acrylamide;
N-(4-(4-amino-3-(4-((3-methylcyclohexyl)oxy)phenyl)-7-oxo-6,7-dihydro-2H-pyrazolo[3,4-d]pyridazin-2-yl)phenyl)acrylamide;
N-(4-(4-amino-3-(4-hydroxyphenyl)-7-oxo-6,7-dihydro-2H-pyrazolo[3,4-d]pyridazin-2-yl)phenyl)acrylamide;
N-(4-(4-amino-3-(4-(methoxymethoxy)phenyl)-7-oxo-6,7-dihydro-2H-pyrazolo[3,4-d]pyridazin-2-yl)phenyl)acrylamide;
N-(4-(4-amino-3-(1-(3,3-difluorocyclopentyl)-1H-indol-5-yl)-7-oxo-6,7-dihydro-2H-pyrazolo[3,4-d]pyridazin-2-yl)phenyl)acrylamide;
4-(4-amino-6-(6-ethynyl-4-methylpyridin-3-yl)pyrazolo[5,1-f][1,2,4]triazin-5-yl)-2-methoxy-N-(2,2,2-trifluoroethyl)benzamide;
4-(4-amino-7-cyano-2-(6-ethynylpyridin-3-yl)-1-methyl-1H-pyrrolo[3,2-c]pyridin-3-yl)-2-methoxy-N-(2,2,2-trifluoroethyl)benzamide;
4-(4-amino-7-cyano-2-(6-ethynylpyridin-3-yl)-1-methyl-1H-pyrrolo[3,2-c]pyridin-3-yl)-N-cyclopropyl-2-methoxybenzamide;
N-(4-(4-amino-5-(4-(cyclopentyloxy)-3-methoxyphenyl)pyrazolo[5,1-f][1,2,4]triazin-6-yl)phenyl)acrylamide;
4-(4-amino-6-(4-(2-fluoroacrylamido)phenyl)pyrazolo[5,1-f][1,2,4]triazin-5-yl)-2-methoxybenzamide;
4-(6-(4-acrylamidophenyl)-4-aminopyrazolo[5,1-f][1,2,4]triazin-5-yl)-N-(3-fluorocyclobutyl)-2-methoxybenzamide;
4-(4-amino-6-(4-(2-fluoroacrylamido)phenyl)pyrazolo[5,1-f][1,2,4]triazin-5-yl)-N-(3-fluorocyclobutyl)-2-methoxybenzamide;
4-(4-amino-6-(4-(2-fluoroacrylamido)phenyl)pyrazolo[5,1-f][1,2,4]triazin-5-yl)-2-methoxy-N-(1-methylcyclopropyl)benzamide;
4-(6-(4-acrylamido-2-chlorophenyl)-4-aminopyrazolo[5,1-f][1,2,4]triazin-5-yl)-N-cyclopropyl-2-methoxybenzamide;
4-(2-(4-acrylamidophenyl)-4-amino-7-cyano-1-methyl-1H-pyrrolo[3,2-c]pyridin-3-yl)-N-(3,3-difluorocyclobutyl)-2-methoxybenzamide;
4-(2-(4-acrylamidophenyl)-4-amino-7-cyano-1-methyl-1H-pyrrolo[3,2-c]pyridin-3-yl)-N-(2-fluoroethyl)-2-methoxybenzamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)phenyl)acrylamide;
N-(4-(4-amino-7-cyano-3-(3-methoxy-4-((6-methylpyridin-2-yl)oxy)phenyl)-1-methyl-1H-pyrrolo[3,2-c]pyridin-2-yl)phenyl)acrylamide;
4-(2-(1-(1-acryloylpiperidin-4-yl)-1H-pyrazol-4-yl)-4-amino-7-cyano-1-methyl-1H-pyrrolo[3,2-c]pyridin-3-yl)-2-methoxy-N-(2,2,2-trifluoroethyl)benzamide;
4-(4-amino-6-(4-(2-fluoroacrylamido)phenyl)pyrazolo[5,1-f][1,2,4]triazin-5-yl)-N-(2,2-difluorocyclopropyl)-2-methoxybenzamide;
4-(6-(4-acrylamidophenyl)-4-aminopyrazolo[5,1-f][1,2,4]triazin-5-yl)-N-(2-fluoroethyl)-2-methoxybenzamide;
4-(4-amino-6-(4-(2-fluoroacrylamido)phenyl)pyrazolo[5,1-f][1,2,4]triazin-5-yl)-N-(2-fluoroethyl)-2-methoxybenzamide;
4-(6-(4-acrylamidophenyl)-4-aminopyrazolo[5,1-f][1,2,4]triazin-5-yl)-N-(2,2-difluoroethyl)-2-methoxybenzamide;
4-(4-amino-6-(4-(2-fluoroacrylamido)phenyl)pyrazolo[5,1-f][1,2,4]triazin-5-yl)-N-(2,2-difluoroethyl)-2-methoxybenzamide;
4-(2-(4-acrylamidophenyl)-4-amino-7-cyano-1-methyl-1H-pyrrolo[3,2-c]pyridin-3-yl)-N-(2,2-difluoroethyl)-2-methoxybenzamide;
4-(4-amino-6-(2-chloro-4-(2-fluoroacrylamido)phenyl)pyrazolo[5,1-f][1,2,4]triazin-5-yl)-N-cyclopropyl-2-methoxybenzamide;
4-(2-(1-acryloylpyrrolidin-3-yl)-4-amino-7-cyano-1-methyl-1H-pyrrolo[3,2-c]pyridin-3-yl)-2-methoxy-N-(2,2,2-trifluoroethyl)benzamide;
4-(6-(1-(1-acryloylpiperidin-4-yl)-1H-pyrazol-4-yl)-4-aminopyrazolo[5,1-f][1,2,4]triazin-5-yl)-2-methoxy-N-(2,2,2-trifluoroethyl)benzamide;
N-(4-(4-amino-7-oxo-3-(4-(pyrrolidin-1-ylmethyl)phenyl)-6,7-dihydro-2H-pyrazolo[3,4-d]pyridazin-2-yl)phenyl)acrylamide;
4-amino-3-(4-(cyclohexyloxy)phenyl)-2-(6-ethynylpyridin-3-yl)-2,6-dihydro-7H-pyrazolo[3,4-d]pyridazin-7-one;
4-(4-amino-6-(4-(2-fluoroacrylamido)phenyl)pyrazolo[5,1-f][1,2,4]triazin-5-yl)-N-((1-fluorocyclopropyl)methyl)-2-methoxybenzamide;
N-(4-(4-amino-5-(3-fluoro-4-((4-methylpyrimidin-2-yl)oxy)phenyl)pyrazolo[5,1-f][1,2,4]triazin-6-yl)phenyl)acrylamide;
N-(4-(4-amino-5-(4-cyclobutoxyphenyl)pyrazolo[5,1-f][1,2,4]triazin-6-yl)phenyl)-2-fluoroacrylamide;
N-(4-(4-amino-5-(4-cyclobutoxyphenyl)pyrazolo[5,1-f][1,2,4]triazin-6-yl)phenyl)methacrylamide;
N-(4-(4-amino-5-(4-cyclobutoxy-3-fluorophenyl)pyrazolo[5,1-f][1,2,4]triazin-6-yl)phenyl)acrylamide;
4-(6-(1-acryloylpyrrolidin-3-yl)-4-aminopyrazolo[5,1-f][1,2,4]triazin-5-yl)-2-methoxy-N-(2,2,2-trifluoroethyl)benzamide;
4-(2-(4-acrylamidophenyl)-4-amino-7-cyano-1-methyl-1H-pyrrolo[3,2-c]pyridin-3-yl)-N-((1-fluorocyclopropyl)methyl)-2-methoxybenzamide;
4-(2-(4-acrylamido-3-fluorophenyl)-4-amino-7-cyano-1-methyl-1H-pyrrolo[3,2-c]pyridin-3-yl)-N-cyclopropyl-2-methoxybenzamide;
4-(2-(4-acrylamido-3-fluorophenyl)-4-amino-7-cyano-1-methyl-1H-pyrrolo[3,2-c]pyridin-3-yl)-2-methoxy-N-(2,2,2-trifluoroethyl)benzamide;
4-(2-(1-acryloyl-2,5-dihydro-1H-pyrrol-3-yl)-4-amino-7-cyano-1-methyl-1H-pyrrolo[3,2-c]pyridin-3-yl)-2-methoxy-N-(2,2,2-trifluoroethyl)benzamide;
N-(4-(4-amino-3-bromo-7-cyano-1-methyl-1H-pyrrolo[3,2-c]pyridin-2-yl)phenyl)-2-fluoroacrylamide;
1-(4-acrylamidophenyl)-3-amino-5-(3-methoxy-4-((6-methylpyridin-2-yl)oxy)phenyl)-1H-pyrazole-4-carboxamide;
N-(4-(4-amino-7-oxo-3-(4-(pyrrolidin-1-yl)phenyl)-6,7-dihydro-2H-pyrazolo[3,4-d]pyridazin-2-yl)phenyl)acrylamide;
N-(4-(4-amino-3-(3-methoxy-4-((6-methylpyridin-2-yl)oxy)phenyl)-2H-pyrazolo[3,4-d]pyrimidin-2-yl)phenyl)acrylamide;
N-(4-(4-amino-5-(6-cyclobutoxypyridin-3-yl)pyrazolo[5,1-f][1,2,4]triazin-6-yl)phenyl)acrylamide;
N-(4-(4-amino-5-(4-cyclobutoxyphenyl)pyrazolo[5,1-f][1,2,4]triazin-6-yl)-3-fluorophenyl)acrylamide;
N-(4-(4-amino-5-(4-cyclobutoxyphenyl)pyrazolo[5,1-f][1,2,4]triazin-6-yl)-3-methoxyphenyl)acrylamide;
N-(4-(4-amino-5-(4-(cyclopropylmethoxy)phenyl)pyrazolo[5,1-f][1,2,4]triazin-6-yl)phenyl)acrylamide;
N-(4-(4-amino-5-(4-cyclopropoxyphenyl)pyrazolo[5,1-f][1,2,4]triazin-6-yl)phenyl)acrylamide;
N-(4-(4-amino-5-(4-(cyclohexylthio)phenyl)pyrazolo[5,1-f][1,2,4]triazin-6-yl)phenyl)acrylamide;
N-(4-(4-amino-3-bromo-7-cyano-1-methyl-1H-pyrrolo[3,2-c]pyridin-2-yl)phenyl)acrylamide;
N-(4-(4-amino-5-(4-(2,2,2-trifluoroethoxy)phenyl)pyrazolo[5,1-f][1,2,4]triazin-6-yl)phenyl)acrylamide;
N-(4-(4-amino-5-(4-isopropoxyphenyl)pyrazolo[5,1-f][1,2,4]triazin-6-yl)phenyl)acrylamide;
N-(4-(4-amino-5-(4-(oxetan-3-yloxy)phenyl)pyrazolo[5,1-f][1,2,4]triazin-6-yl)phenyl)acrylamide;
N-(4-(4-amino-5-(4-(cyclohexyloxy)phenyl)pyrazolo[5,1-f][1,2,4]triazin-6-yl)phenyl)acrylamide;
N-(4-(4-amino-5-(4-((tetrahydro-2H-pyran-4-yl)oxy)phenyl)pyrazolo[5,1-f][1,2,4]triazin-6-yl)phenyl)acrylamide;
N-(4-(4-amino-5-(4-((3-methylcyclohexyl)oxy)phenyl)pyrazolo[5,1-f][1,2,4]triazin-6-yl)phenyl)acrylamide;
N-(4-(4-amino-5-(4-((4,4-dimethylcyclohexyl)oxy)phenyl)pyrazolo[5,1-f][1,2,4]triazin-6-yl)phenyl)acrylamide;
N-(4-(6-(4-acrylamidophenyl)-4-aminopyrazolo[5,1-f][1,2,4]triazin-5-yl)-2-methoxyphenyl)cyclopropanecarboxamide;
N-(4-(4-amino-5-(4-(1-(cyclopropylamino)-2,2,2-trifluoroethyl)-3-methoxyphenyl) pyrazolo[5,1-f][1,2,4]triazin-6-yl)phenyl)acrylamide;
4-(4-amino-6-(2-fluoro-4-(2-fluoroacrylamido)phenyl)pyrazolo[5,1-f][1,2,4]triazin-5-yl)-N-(3,3-difluorocyclobutyl)-2-methoxybenzamide;
4-(4-amino-7-cyano-2-(4-(2-fluoroacrylamido)phenyl)-1H-pyrrolo[3,2-c]pyridin-3-yl)-N-cyclopropyl-2-methoxybenzamide;
4-(2-(4-acrylamidophenyl)-4-amino-7-cyano-1-(2-fluoroethyl)-1H-pyrrolo[3,2-c]pyridin-3-yl)-N-cyclopropyl-2-methoxybenzamide;
N-(4-(2-(4-acrylamidophenyl)-4-amino-7-cyano-1-methyl-1H-pyrrolo[3,2-c]pyridin-3-yl)-2-methoxyphenyl)cyclopropanecarboxamide;
N-(4-(4-amino-7-cyano-3-(4-((5-fluoropyrimidin-2-yl)oxy)phenyl)-1-methyl-1H-pyrrolo[3,2-c]pyridin-2-yl)phenyl)acrylamide;
N-(4-(4-amino-5-(4-((3,3-difluorocyclopentyl)oxy)phenyl)pyrazolo[5,1-f][1,2,4]triazin-6-yl)phenyl)acrylamide;
N-(4-(4-amino-5-(4-cyclobutoxy-3-methoxyphenyl)pyrazolo[5,1-f][1,2,4]triazin-6-yl)phenyl)acrylamide;
N-(4-(4-amino-5-(4-(1-(cyclopropylamino)-2,2,2-trifluoroethyl)-3-methoxyphenyl) pyrazolo[5,1-f][1,2,4]triazin-6-yl)phenyl)-2-fluoroacrylamide;
4-(2-(4-acrylamidophenyl)-4-amino-7-cyano-1-ethyl-1H-pyrrolo[3,2-c]pyridin-3-yl)-N-cyclopropyl-2-methoxybenzamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)phenyl)acrylamide;
N-(4-(4-amino-7-cyano-3-(4-(1-(cyclopropylamino)-2,2,2-trifluoroethyl)-3-methoxyphenyl)-1-methyl-1H-pyrrolo[3,2-c]pyridin-2-yl)phenyl)acrylamide;
N-(4-(4-amino-5-(4-((4,4-difluorocyclohexyl)oxy)phenyl)pyrazolo[5,1-f][1,2,4]triazin-6-yl)phenyl)acrylamide;
N-(4-(4-amino-5-(4-((1-methylazetidin-3-yl)oxy)phenyl)pyrazolo[5,1-f][1,2,4]triazin-6-yl)phenyl)acrylamide;
N-(4-(4-amino-5-(4-(3-(dimethylamino)cyclobutoxy)phenyl)pyrazolo[5,1-f][1,2,4]triazin-6-yl)phenyl)acrylamide;
4-(2-(4-acrylamidophenyl)-4-amino-7-cyano-1H-pyrrolo[3,2-c]pyridin-3-yl)-2-methoxy-N-(2,2,2-trifluoroethyl)benzamide;
4-(4-amino-6-(2-fluoro-4-(2-fluoroacrylamido)phenyl)pyrazolo[5,1-f][1,2,4]triazin-5-yl)-N-(3-fluorocyclobutyl)-2-methoxybenzamide;
4-(4-amino-6-(4-(2-fluoroacrylamido)-2-methoxyphenyl)pyrazolo[5,1-f][1,2,4]triazin-5-yl)-N-((1-fluorocyclopropyl)methyl)-2-methoxybenzamide;
4-(4-amino-6-(4-(2-fluoroacrylamido)-2-methoxyphenyl)pyrazolo[5,1-f][1,2,4]triazin-5-yl)-N-(2,2-difluoroethyl)-2-methoxybenzamide;
4-(4-amino-6-(6-(2-fluoroacrylamido)pyridin-3-yl)pyrazolo[5,1-f][1,2,4]triazin-5-yl)-2-methoxy-N-(2,2,2-trifluoroethyl)benzamide;
4-(4-amino-6-(3-fluoro-4-(2-fluoroacrylamido)phenyl)pyrazolo[5,1-f][1,2,4]triazin-5-yl)-2-methoxy-N-(2,2,2-trifluoroethyl)benzamide;
4-(4-amino-6-(4-(2-fluoroacrylamido)-3-methoxyphenyl)pyrazolo[5,1-f][1,2,4]triazin-5-yl)-2-methoxy-N-(2,2,2-trifluoroethyl)benzamide;
4-(4-amino-6-(4-(2-fluoroacrylamido)-2-methoxyphenyl)pyrazolo[5,1-f][1,2,4]triazin-5-yl)-N-(3-fluorocyclobutyl)-2-methoxybenzamide;
4-(4-amino-6-(2-fluoro-4-(2-fluoroacrylamido)phenyl)pyrazolo[5,1-f][1,2,4]triazin-5-yl)-2-methoxy-N-(1-methylcyclopropyl)benzamide;
4-(4-amino-6-(4-(2-fluoroacrylamido)-2-methoxyphenyl)pyrazolo[5,1-f][1,2,4]triazin-5-yl)-2-methoxy-N-(1-methylcyclopropyl)benzamide;
4-(4-amino-6-(2-fluoro-4-(2-fluoroacrylamido)phenyl)pyrazolo[5,1-f][1,2,4]triazin-5-yl)-N-((1-fluorocyclopropyl)methyl)-2-methoxybenzamide;
4-(4-amino-6-(4-(2-fluoroacrylamido)-2-methoxyphenyl)pyrazolo[5,1-f][1,2,4]triazin-5-yl)-N-(2,2-difluorocyclopropyl)-2-methoxybenzamide;
N-(4-(4-amino-5-(4-(bicyclo[3.1.0]hexan-3-yloxy)phenyl)pyrazolo[5,1-f][1,2,4]triazin-6-yl)phenyl)acrylamide;
N-(4-(4-amino-5-(4-((3-fluorocyclopentyl)oxy)phenyl)pyrazolo[5,1-f][1,2,4]triazin-6-yl)phenyl)acrylamide N-(5-(4-amino-5-(4-cyclobutoxyphenyl)pyrazolo[5,1-f][1,2,4]triazin-6-yl)pyridin-2-yl)acrylamide;
N-(4-(6-(4-acrylamidophenyl)-4-aminopyrazolo[5,1-f][1,2,4]triazin-5-yl)phenyl)cyclopropanecarboxamide;
N-(4-(4-amino-5-(4-(pyrrolidin-1-yl)phenyl)pyrazolo[5,1-f][1,2,4]triazin-6-yl)phenyl)acrylamide;
N-(4-(2-(4-acrylamidophenyl)-4-amino-7-cyano-1-methyl-1H-pyrrolo[3,2-c]pyridin-3-yl)-2-methoxyphenyl)cyclobutanecarboxamide;
N-(4-(2-(4-acrylamidophenyl)-4-amino-7-cyano-1-methyl-1H-pyrrolo[3,2-c]pyridin-3-yl)-2-methoxyphenyl)cyclopentanecarboxamide;
4-(4-amino-7-cyano-2-(2-fluoro-4-(2-fluoroacrylamido)phenyl)-1H-pyrrolo[3,2-c]pyridin-3-yl)-N-cyclopropyl-2-methoxybenzamide;
4-(6-(4-acrylamidophenyl)-4-aminopyrazolo[5,1-f][1,2,4]triazin-5-yl)-N-(1-cyanocyclopropyl)-2-methoxybenzamide;
4-(6-(4-acrylamidophenyl)-4-aminopyrazolo[5,1-f][1,2,4]triazin-5-yl)-N-(bicyclo[1.1.1]pentan-1-yl)-2-methoxybenzamide;
4-(4-amino-6-(4-(2-fluoroacrylamido)phenyl)pyrazolo[5,1-f][1,2,4]triazin-5-yl)-N-(bicyclo[1.1.1]pentan-1-yl)-2-methoxybenzamide;
4-(4-amino-6-(2-fluoro-4-(2-fluoroacrylamido)phenyl)pyrazolo[5,1-f][1,2,4]triazin-5-yl)-N-(2,2-difluorocyclopropyl)-2-methoxybenzamide;
N-(4-(6-(4-acrylamidophenyl)-4-aminopyrazolo[5,1-f][1,2,4]triazin-5-yl)-2-methoxyphenyl)cyclobutanecarboxamide;
N-(4-(4-amino-5-(4-(3-methoxycyclobutoxy)phenyl)pyrazolo[5,1-f][1,2,4]triazin-6-yl)phenyl)acrylamide;
N-(4-(4-amino-5-(4-(spiro[2.3]hexan-5-yloxy)phenyl)pyrazolo[5,1-f][1,2,4]triazin-6-yl)phenyl)acrylamide;
N-(4-(4-amino-5-(3-fluoro-4-((4-methylpyrimidin-2-yl)oxy)phenyl)pyrazolo[5,1-f][1,2,4]triazin-6-yl)phenyl)-2-fluoroacrylamide;
4-(2-(4-acrylamido-2-methoxyphenyl)-4-amino-7-cyano-1H-pyrrolo[3,2-c]pyridin-3-yl)-N-(3,3-difluorocyclobutyl)-2-methoxybenzamide;
N-(4-(4-amino-7-cyano-3-(3-methoxy-4-propionamidophenyl)-1-methyl-1H-pyrrolo[3,2-c]pyridin-2-yl)phenyl)acrylamide;
4-(2-(4-acrylamidophenyl)-4-amino-7-cyano-1-(2-fluoroethyl)-1H-pyrrolo[3,2-c]pyridin-3-yl)-N-(3,3-difluorocyclobutyl)-2-methoxybenzamide;
4-(4-amino-6-(4-(2-fluoroacrylamido)phenyl)pyrazolo[5,1-f][1,2,4]triazin-5-yl)-N-(1-cyanocyclopropyl)-2-methoxybenzamide;
N-(4-(4-amino-5-(4-(cyclobutylamino)phenyl)pyrazolo[5,1-f][1,2,4]triazin-6-yl)phenyl)acrylamide;
4-(2-(4-acrylamido-2-fluorophenyl)-4-amino-7-cyano-1H-pyrrolo[3,2-c]pyridin-3-yl)-N-(3,3-difluorocyclobutyl)-2-methoxybenzamide;
4-(2-(4-acrylamidophenyl)-4-amino-7-cyano-1-isopropyl-1H-pyrrolo[3,2-c]pyridin-3-yl)-N-(3,3-difluorocyclobutyl)-2-methoxybenzamide;
N-(4-(2-(4-acrylamidophenyl)-4-amino-7-cyano-1-methyl-1H-pyrrolo[3,2-c]pyridin-3-yl)-2-methoxyphenyl)-N-methylcyclopropanecarboxamide;
N-(4-(2-(4-acrylamidophenyl)-4-amino-7-cyano-1-methyl-1H-pyrrolo[3,2-c]pyridin-3-yl)phenyl)cyclopropanecarboxamide;
N-(4-(2-(4-acrylamidophenyl)-4-amino-7-cyano-1-methyl-1H-pyrrolo[3,2-c]pyridin-3-yl)-2-methoxyphenyl)-3,3-difluorocyclobutane-1-carboxamide;
N-(4-(2-(4-acrylamidophenyl)-4-amino-7-cyano-1-methyl-1H-pyrrolo[3,2-c]pyridin-3-yl)-2-methoxyphenyl)-1-methylcyclopropane-1-carboxamide;
N-(4-(2-(4-acrylamidophenyl)-4-amino-7-cyano-1-methyl-1H-pyrrolo[3,2-c]pyridin-3-yl)-2-methoxyphenyl)-1-fluorocyclopropane-1-carboxamide;
N-(4-(2-(4-acrylamidophenyl)-4-amino-7-cyano-1-methyl-1H-pyrrolo[3,2-c]pyridin-3-yl)-2-methoxyphenyl)-1-(trifluoromethyl)cyclopropane-1-carboxamide;
4-(4-amino-6-(2-fluoro-4-(2-fluoroacrylamido)phenyl)pyrazolo[5,1-f][1,2,4]triazin-5-yl)-N-(2,2-difluoroethyl)-2-methoxybenzamide;
N-(4-(4-amino-7-cyano-3-(4-cyclobutoxy-3-methoxyphenyl)-1-methyl-1H-pyrrolo[3,2-c]pyridin-2-yl)phenyl)acrylamide;
N-(4-(2-(4-acrylamidophenyl)-4-amino-7-cyano-1-methyl-1H-pyrrolo[3,2-c]pyridin-3-yl)-2-methoxyphenyl)-2,2-difluorocyclopropane-1-carboxamide;
N-(4-(4-amino-7-cyano-3-(4-(3-cyclopropylureido)-3-methoxyphenyl)-1-methyl-1H-pyrrolo[3,2-c]pyridin-2-yl)phenyl)acrylamide;
4-(4-amino-6-(4-(2-fluoroacrylamido)phenyl)pyrazolo[5,1-f][1,2,4]triazin-5-yl)-2-methoxy-N-(1,1,1-trifluoropropan-2-yl)benzamide;
4-(4-amino-6-(3-((dimethylamino)methyl)-4-(2-fluoroacrylamido)phenyl)pyrazolo[5,1-f][1,2,4]triazin-5-yl)-2-methoxy-N-(2,2,2-trifluoroethyl)benzamide;
N-(4-(4-amino-5-(4-(azetidin-1-yl)phenyl)pyrazolo[5,1-f][1,2,4]triazin-6-yl)phenyl)acrylamide;
N-(4-(4-amino-5-(4-(3-cyclopropylureido)-3-methoxyphenyl)pyrazolo[5,1-f][1,2,4]triazin-6-yl)phenyl)acrylamide;
N-(4-(6-(4-acrylamidophenyl)-4-aminopyrazolo[5,1-f][1,2,4]triazin-5-yl)-2-methoxyphenyl)azetidine-1-carboxamide;
N-(4-(4-amino-7-cyano-3-(4-(cyclobutylamino)phenyl)-1-methyl-1H-pyrrolo[3,2-c]pyridin-2-yl)phenyl)acrylamide;
N-(4-(2-(4-acrylamidophenyl)-4-amino-7-cyano-1-methyl-1H-pyrrolo[3,2-c]pyridin-3-yl)-2-methoxyphenyl)azetidine-1-carboxamide;
4-(2-(4-acrylamido-3-((dimethylamino)methyl)phenyl)-4-amino-7-cyano-1-methyl-1H-pyrrolo[3,2-c]pyridin-3-yl)-2-methoxy-N-(2,2,2-trifluoroethyl)benzamide;
N-(4-(4-amino-7-cyano-1-methyl-3-(4-((4-methylpyrimidin-2-yl)oxy)phenyl)-1H-pyrrolo[3,2-c]pyridin-2-yl)phenyl)-2-fluoroacrylamide;
4-(4-amino-7-cyano-2-(4-(2-fluoroacrylamido)phenyl)-1H-pyrrolo[3,2-c]pyridin-3-yl)-2-methoxy-N-(2,2,2-trifluoroethyl)benzamide;
4-(4-amino-7-cyano-2-(4-(2-fluoroacrylamido)phenyl)-1H-pyrrolo[3,2-c]pyridin-3-yl)-2-methoxy-N-(1-(trifluoromethyl)cyclopropyl)benzamide;
4-(2-(4-acrylamidophenyl)-4-amino-7-cyano-1-(2-methoxyethyl)-1H-pyrrolo[3,2-c]pyridin-3-yl)-N-(3,3-difluorocyclobutyl)-2-methoxybenzamide;
4-(2-(4-acrylamidophenyl)-4-amino-7-cyano-1-(2-fluoroethyl)-1H-pyrrolo[3,2-c]pyridin-3-yl)-2-methoxy-N-(2,2,2-trifluoroethyl)benzamide;
4-(2-(4-acrylamidophenyl)-4-amino-7-cyano-1-methyl-1H-pyrrolo[3,2-c]pyridin-3-yl)-2-methoxy-N-(1-(trifluoromethyl)cyclopropyl)benzamide;
4-(6-(4-acrylamidophenyl)-4-aminopyrazolo[5,1-f][1,2,4]triazin-5-yl)-2-methoxy-N-(1-(trifluoromethyl)cyclopropyl)benzamide;
4-(4-amino-6-(4-(2-fluoroacrylamido)phenyl)pyrazolo[5,1-f][1,2,4]triazin-5-yl)-2-methoxy-N-(1-(trifluoromethyl)cyclopropyl)benzamide;
4-(4-amino-6-(4-(2-fluoroacrylamido)phenyl)pyrazolo[5,1-f][1,2,4]triazin-5-yl)-2-methoxy-N-((1-(trifluoromethyl)cyclopropyl)methyl)benzamide;
4-(4-amino-6-(4-(2-fluoroacrylamido)phenyl)pyrazolo[5,1-f][1,2,4]triazin-5-yl)-2-methoxy-N-(3,3,3-trifluoropropyl)benzamide;
4-(6-(4-acrylamido-3-((dimethylamino)methyl)phenyl)-4-aminopyrazolo[5,1-f][1,2,4]triazin-5-yl)-2-methoxy-N-(2,2,2-trifluoroethyl)benzamide;
4-(6-(4-acrylamido-2-((dimethylamino)methyl)phenyl)-4-aminopyrazolo[5,1-f][1,2,4]triazin-5-yl)-2-methoxy-N-(2,2,2-trifluoroethyl)benzamide;
N-(4-(4-amino-5-(3-methoxy-4-(pyrrolidin-1-yl)phenyl)pyrazolo[5,1-f][1,2,4]triazin-6-yl)phenyl)acrylamide;
N-(4-(4-amino-5-(4-(cyclobutylamino)-3-methoxyphenyl)pyrazolo[5,1-f][1,2,4]triazin-6-yl)phenyl)acrylamide;
N-(4-(4-amino-7-cyano-1-methyl-3-(4-(pyrrolidin-1-yl)phenyl)-1H-pyrrolo[3,2-c]pyridin-2-yl)phenyl)acrylamide;
N-(4-(4-amino-7-cyano-3-(3-methoxy-4-(pyrrolidin-1-yl)phenyl)-1-methyl-1H-pyrrolo[3,2-c]pyridin-2-yl)phenyl)acrylamide;
N-(4-(4-amino-7-cyano-3-(4-(cyclobutylamino)-3-methoxyphenyl)-1-methyl-1H-pyrrolo[3,2-c]pyridin-2-yl)phenyl)acrylamide;
4-(2-(4-acrylamidophenyl)-4-amino-7-cyano-1-(2-methoxyethyl)-1H-pyrrolo[3,2-c]pyridin-3-yl)-2-methoxy-N-(2,2,2-trifluoroethyl)benzamide;
4-(4-amino-7-cyano-2-(4-(2-fluoroacrylamido)phenyl)-1H-pyrrolo[3,2-c]pyridin-3-yl)-N-(3,3-difluorocyclobutyl)-2-methoxybenzamide;
4-(4-amino-7-cyano-2-(4-(2-fluoroacrylamido)phenyl)-1H-pyrrolo[3,2-c]pyridin-3-yl)-N-(2,2-difluorocyclopropyl)-2-methoxybenzamide;
4-(4-amino-7-cyano-2-(4-(2-fluoroacrylamido)phenyl)-1H-pyrrolo[3,2-c]pyridin-3-yl)-2-methoxy-N-(1-methylcyclopropyl)benzamide;
N-(4-(4-amino-5-(1-methyl-1H-indol-5-yl)pyrazolo[5,1-f][1,2,4]triazin-6-yl)phenyl)acrylamide;
N-(4-(4-amino-5-(1-methyl-1H-indol-5-yl)pyrazolo[5,1-f][1,2,4]triazin-6-yl)phenyl)-2-fluoroacrylamide;
4-(4-amino-6-(4-(2-fluoroacrylamido)phenyl)pyrazolo[5,1-f][1,2,4]triazin-5-yl)-N-(2,2,2-trifluoroethyl)benzamide;
N-(4-(4-amino-5-(4-((4-methylpyrimidin-2-yl)oxy)phenyl)pyrazolo[5,1-f][1,2,4]triazin-6-yl)phenyl)-2-fluoroacrylamide;
N-(4-(4-amino-7-cyano-1-methyl-3-(1-methyl-1H-indol-5-yl)-1H-pyrrolo[3,2-c]pyridin-2-yl)phenyl)acrylamide;
N-(4-(4-amino-3-(benzo[b]thiophen-2-yl)-7-cyano-1-methyl-1H-pyrrolo[3,2-c]pyridin-2-yl)phenyl)acrylamide;
4-(2-(4-acrylamidophenyl)-4-amino-7-cyano-1-isopropyl-1H-pyrrolo[3,2-c]pyridin-3-yl)-2-methoxy-N-(2,2,2-trifluoroethyl)benzamide;
4-(2-(4-acrylamidophenyl)-4-amino-7-cyano-1-(2,2,2-trifluoroethyl)-1H-pyrrolo[3,2-c]pyridin-3-yl)-2-methoxy-N-(2,2,2-trifluoroethyl)benzamide;
4-(4-amino-6-(4-(2-fluoroacrylamido)phenyl)pyrazolo[5,1-f][1,2,4]triazin-5-yl)-N-(3-fluorobicyclo[1.1.1]pentan-1-yl)-2-methoxybenzamide;
N-(4-(4-amino-5-(benzo[b]thiophen-2-yl)pyrazolo[5,1-f][1,2,4]triazin-6-yl)phenyl)-2-fluoroacrylamide;
N-(4-(4-amino-5-(benzo[b]thiophen-2-yl)pyrazolo[5,1-f][1,2,4]triazin-6-yl)phenyl)acrylamide;
4-(4-amino-6-(4-(2-fluoroacrylamido)phenyl)pyrazolo[5,1-f][1,2,4]triazin-5-yl)-N-((3,3-difluorocyclobutyl)methyl)-2-methoxybenzamide;
4-(4-amino-6-(4-(2-fluoroacrylamido)phenyl)pyrazolo[5,1-f][1,2,4]triazin-5-yl)-2-fluoro-N-(2,2,2-trifluoroethyl)benzamide;
N-(4-(4-amino-5-(3-fluoro-4-((5-fluoropyrimidin-2-yl)oxy)phenyl)pyrazolo[5,1-f][1,2,4]triazin-6-yl)phenyl)-2-fluoroacrylamide;
4-(4-amino-6-(4-(2-fluoroacrylamido)phenyl)pyrazolo[5,1-f][1,2,4]triazin-5-yl)-2-methoxy-N-(3-(trifluoromethyl)bicyclo[1.1.1]pentan-1-yl)benzamide;
4-(4-amino-6-(4-(2-fluoroacrylamido)phenyl)pyrazolo[5,1-f][1,2,4]triazin-5-yl)-N-(1,1-difluoropropan-2-yl)-2-methoxybenzamide;
N-(4-(6-(4-acrylamidophenyl)-4-aminopyrazolo[5,1-f][1,2,4]triazin-5-yl)-2-methoxyphenyl)-3,3-difluorocyclobutane-1-carboxamide;
4-(2-(4-acrylamidophenyl)-4-amino-7-cyano-1-methyl-1H-pyrrolo[3,2-c]pyridin-3-yl)-N-(bicyclo[1.1.1]pentan-1-yl)-2-methoxybenzamide;
4-(2-(4-acrylamidophenyl)-4-amino-7-cyano-1-(2,2,2-trifluoroethyl)-1H-pyrrolo[3,2-c]pyridin-3-yl)-N-cyclopropyl-2-methoxybenzamide;
N-(4-(2-(4-acrylamidophenyl)-4-amino-7-cyano-1-methyl-1H-pyrrolo[3,2-c]pyridin-3-yl)-2-fluorophenyl)cyclopropanecarboxamide;
4-(3-acrylamido-12-amino-9-cyano-6,7-dihydro-5H-benzo[c]pyrido[3′,4′:4,5]pyrrolo[1,2-a]azepin-13-yl)-N-(3,3-difluorocyclobutyl)-2-methoxybenzamide;
4-(4-amino-6-(4-(2-fluoroacrylamido)phenyl)pyrazolo[5,1-f][1,2,4]triazin-5-yl)-2-(methylthio)-N-(2,2,2-trifluoroethyl)benzamide;
N-(4-(4-amino-5-(2-(4-methoxyphenyl)thiazol-5-yl)pyrazolo[5,1-f][1,2,4]triazin-6-yl)phenyl)-2-fluoroacrylamide;
N-(4-(4-amino-5-(2-(4-methoxyphenyl)thiazol-5-yl)pyrazolo[5,1-f][1,2,4]triazin-6-yl)phenyl)acrylamide;
4-(2-(4-acrylamidophenyl)-4-amino-7-cyano-1-cyclopropyl-1H-pyrrolo[3,2-c]pyridin-3-yl)-2-methoxy-N-(2,2,2-trifluoroethyl)benzamide;
4-(2-(4-acrylamidophenyl)-4-amino-7-cyano-1-methyl-1H-pyrrolo[3,2-c]pyridin-3-yl)-2-(methylthio)-N-(2,2,2-trifluoroethyl)benzamide;
N-(4-(4-amino-5-(3-methoxy-4-(2-oxo-2-((2,2,2-trifluoroethyl)amino)ethyl)phenyl) pyrazolo[5,1-f][1,2,4]triazin-6-yl)phenyl)acrylamide;
N-(4-(4-amino-7-cyano-3-(3-methoxy-4-(2-oxo-2-((2,2,2-trifluoroethyl)amino)ethyl)phenyl)-1-methyl-1H-pyrrolo[3,2-c]pyridin-2-yl)phenyl)acrylamide;
4-(4-amino-6-(2-fluoro-4-(2-fluoroacrylamido)phenyl)pyrazolo[5,1-f][1,2,4]triazin-5-yl)-2-methoxy-N-(1-(trifluoromethyl)cyclopropyl)benzamide;
4-(4-amino-6-(2-fluoro-4-(2-fluoroacrylamido)phenyl)pyrazolo[5,1-f][1,2,4]triazin-5-yl)-N-(bicyclo[1.1.1]pentan-1-yl)-2-methoxybenzamide;
4-(4-amino-6-(2-fluoro-4-(2-fluoroacrylamido)phenyl)pyrazolo[5,1-f][1,2,4]triazin-5-yl)-2-methoxy-N-((1-(trifluoromethyl)cyclopropyl)methyl)benzamide;
4-(2-(4-acrylamidophenyl)-4-amino-7-cyano-1-cyclopropyl-1H-pyrrolo[3,2-c]pyridin-3-yl)-N-cyclopropyl-2-methoxybenzamide;
N-(4-(4-amino-5-(3-methoxy-4-(2-oxo-2-((2,2,2-trifluoroethyl)amino)ethyl)phenyl) pyrazolo[5,1-f][1,2,4]triazin-6-yl)phenyl)-2-fluoroacrylamide;
4-(4-amino-6-(2-fluoro-4-(2-fluoroacrylamido)phenyl)pyrazolo[5,1-f][1,2,4]triazin-5-yl)-2-methoxy-N-((1-(trifluoromethyl)cyclopropyl)methyl)benzamide;
4-(4-amino-6-(4-(2-fluoroacrylamido)phenyl)pyrazolo[5,1-f][1,2,4]triazin-5-yl)-N-(2,2-difluoropropyl)-2-methoxybenzamide;
N-(4-(4-amino-5-(4-(azetidine-1-carbonyl)-3-methoxyphenyl)pyrazolo[5,1-f][1,2,4]triazin-6-yl)phenyl)-2-fluoroacrylamide;
N-(4-(4-amino-5-(3-fluoro-4-((6-methylpyridin-2-yl)oxy)phenyl)pyrazolo[5,1-f][1,2,4]triazin-6-yl)phenyl)-2-fluoroacrylamide;
N-(4-(4-amino-5-(3-fluoro-4-((6-methylpyridin-2-yl)oxy)phenyl)pyrazolo[5,1-f][1,2,4]triazin-6-yl)phenyl)acrylamide;
N-(4-(4-amino-5-(3-fluoro-4-((2-methylpyrimidin-4-yl)oxy)phenyl)pyrazolo[5,1-f][1,2,4]triazin-6-yl)phenyl)-2-fluoroacrylamide;
N-(4-(4-amino-5-(3-fluoro-4-((2-methylpyrimidin-4-yl)oxy)phenyl)pyrazolo[5,1-f][1,2,4]triazin-6-yl)phenyl)acrylamide;
N-(4-(4-amino-5-(1-oxo-2-(2,2,2-trifluoroethyl)-1,2-dihydroisoquinolin-6-yl)pyrazolo[5,1-f][1,2,4]triazin-6-yl)phenyl)acrylamide;
N-(4-(4-amino-5-(1-oxo-2-(2,2,2-trifluoroethyl)isoindolin-5-yl)pyrazolo[5,1-f][1,2,4]triazin-6-yl)phenyl)acrylamide;
4-(4-amino-6-(4-(2-fluoroacrylamido)phenyl)pyrazolo[5,1-f][1,2,4]triazin-5-yl)-N-((1-fluorocyclopropyl)methyl)-2-(methoxy-d3)benzamide;
N-(4-(4-amino-5-(3-fluoro-4-((6-methylpyrazin-2-yl)oxy)phenyl)pyrazolo[5,1-f][1,2,4]triazin-6-yl)phenyl)-2-fluoroacrylamide;
N-(4-(4-amino-5-(3-fluoro-4-((6-methylpyrazin-2-yl)oxy)phenyl)pyrazolo[5,1-f][1,2,4]triazin-6-yl)phenyl)acrylamide;
N-(4-(4-amino-5-(3-fluoro-4-((5-methylpyrimidin-2-yl)oxy)phenyl)pyrazolo[5,1-f][1,2,4]triazin-6-yl)phenyl)-2-fluoroacrylamide;
N-(4-(4-amino-5-(3-fluoro-4-((5-methylpyrimidin-2-yl)oxy)phenyl)pyrazolo[5,1-f][1,2,4]triazin-6-yl)phenyl)acrylamide;
N-(4-(4-amino-5-(3-fluoro-4-(pyrimidin-2-yloxy)phenyl)pyrazolo[5,1-f][1,2,4]triazin-6-yl)phenyl)-2-fluoroacrylamide;
N-(4-(4-amino-3-(3-fluoro-4-((4-methylpyrimidin-2-yl)oxy)phenyl)-2H-pyrazolo[3,4-d]pyrimidin-2-yl)phenyl)methacrylamide;
N-(4-(4-amino-5-(3-fluoro-4-((4-methoxypyrimidin-2-yl)oxy)phenyl)pyrazolo[5,1-f][1,2,4]triazin-6-yl)phenyl)-2-fluoroacrylamide;
N-(4-(4-amino-5-(4-((4-(difluoromethyl)pyrimidin-2-yl)oxy)-3-fluorophenyl)pyrazolo[5,1-f][1,2,4]triazin-6-yl)phenyl)-2-fluoroacrylamide;
N-(4-(4-amino-5-(3-fluoro-4-((6-methylpyrimidin-4-yl)oxy)phenyl)pyrazolo[5,1-f][1,2,4]triazin-6-yl)phenyl)-2-fluoroacrylamide;
N-(4-(4-amino-5-(3-fluoro-4-((6-methylpyrimidin-4-yl)oxy)phenyl)pyrazolo[5,1-f][1,2,4]triazin-6-yl)phenyl)acrylamide;
4-(4-amino-6-(4-(2-fluoroacrylamido)phenyl)pyrazolo[5,1-f][1,2,4]triazin-5-yl)-N-((1-fluorocyclopropyl)methyl)benzamide;
N-(4-(4-amino-5-(3-fluoro-4-((4-methylpyrimidin-2-yl)thio)phenyl)pyrazolo[5,1-f][1,2,4]triazin-6-yl)phenyl)-2-fluoroacrylamide;
N-(4-(4-amino-5-(3-fluoro-4-((4-methylpyrimidin-2-yl)thio)phenyl)pyrazolo[5,1-f][1,2,4]triazin-6-yl)phenyl)-2-fluoroacrylamide;
N-(4-(4-amino-5-(4-((4-cyclopropylpyrimidin-2-yl)oxy)-3-fluorophenyl)pyrazolo[5,1-f][1,2,4]triazin-6-yl)phenyl)-2-fluoroacrylamide N-(4-(4-amino-5-(4-((4-cyclopropylpyrimidin-2-yl)oxy)-3-fluorophenyl)pyrazolo[5,1-f][1,2,4]triazin-6-yl)phenyl)acrylamide;
N-(4-(4-amino-5-(3-fluoro-4-(imidazo[1,2-c]pyrimidin-5-yloxy)phenyl)pyrazolo[5,1-f][1,2,4]triazin-6-yl)phenyl)-2-fluoroacrylamide;
N-(4-(4-amino-5-(3-fluoro-4-((4-methylpyrimidin-2-yl)oxy)phenyl)pyrazolo[5,1-f][1,2,4]triazin-6-yl)-3-fluorophenyl)methacrylamide;
4-(4-amino-6-(4-(2-fluoroacrylamido)phenyl)pyrazolo[5,1-f][1,2,4]triazin-5-yl)-2-methoxy-N-phenylbenzamide;
4-(4-amino-6-(4-(2-fluoroacrylamido)phenyl)pyrazolo[5,1-f][1,2,4]triazin-5-yl)-2-methoxy-N-(pyridin-2-yl)benzamide;
N-(4-(4-amino-5-(3-fluoro-4-(methyl(4-methylpyrimidin-2-yl)amino)phenyl) pyrazolo[5,1-f][1,2,4]triazin-6-yl)phenyl)-2-fluoroacrylamide;
N-(4-(4-amino-5-(3-fluoro-4-((4-(trifluoromethyl)pyrimidin-2-yl)oxy)phenyl) pyrazolo[5,1-f][1,2,4]triazin-6-yl)phenyl)-2-fluoroacrylamide;
N-(4-(4-amino-5-(3-fluoro-4-((4-(trifluoromethyl)pyrimidin-2-yl)oxy)phenyl)pyrazolo[5,1-f][1,2,4]triazin-6-yl)phenyl)acrylamide;
4-(4-amino-6-(4-(but-2-ynamido)phenyl)pyrazolo[5,1-f][1,2,4]triazin-5-yl)-2-methoxy-N-(1-(trifluoromethyl)cyclopropyl)benzamide;
4-(4-amino-6-(4-propiolamidophenyl)pyrazolo[5,1-f][1,2,4]triazin-5-yl)-2-methoxy-N-(1-(trifluoromethyl)cyclopropyl)benzamide;
4-(4-amino-6-(4-(2-fluoroacrylamido)phenyl)pyrazolo[5,1-f][1,2,4]triazin-5-yl)-N-(tert-butyl)-2-methoxybenzamide;
N-(4-(4-amino-5-(4-((4-ethylpyrimidin-2-yl)oxy)-3-fluorophenyl)pyrazolo[5,1-f][1,2,4]triazin-6-yl)phenyl)-2-fluoroacrylamide;
N-(4-(4-amino-5-(4-((4-ethylpyrimidin-2-yl)oxy)-3-fluorophenyl)pyrazolo[5,1-f][1,2,4]triazin-6-yl)phenyl)acrylamide;
N-(4-(4-amino-5-(3-fluoro-4-((1-methyl-1H-pyrazol-3-yl)oxy)phenyl)pyrazolo[5,1-f][1,2,4]triazin-6-yl)phenyl)-2-fluoroacrylamide;
N-(4-(4-amino-5-(3-fluoro-4-((1-methyl-1H-pyrazol-3-yl)oxy)phenyl)pyrazolo[5,1-f][1,2,4]triazin-6-yl)phenyl)acrylamide;
N-(4-(4-amino-5-(3-fluoro-4-((4-methylpyrimidin-2-yl)amino)phenyl)pyrazolo[5,1-f][1,2,4]triazin-6-yl)phenyl)-2-fluoroacrylamide;
N-(4-(4-amino-5-(3-fluoro-4-((4-methylpyrimidin-2-yl)amino)phenyl)pyrazolo[5,1-f][1,2,4]triazin-6-yl)phenyl)acrylamide;
N-(4-(4-amino-5-(3-fluoro-4-((4-methylpyridin-2-yl)oxy)phenyl)pyrazolo[5,1-f][1,2,4]triazin-6-yl)phenyl)-2-fluoroacrylamide;
N-(4-(4-amino-5-(3-fluoro-4-((4-methylpyridin-2-yl)oxy)phenyl)pyrazolo[5,1-f][1,2,4]triazin-6-yl)phenyl)acrylamide;
N-(4-(4-amino-5-(4-(4-fluorobenzoyl)-3-methoxyphenyl)pyrazolo[5,1-f][1,2,4]triazin-6-yl)phenyl)acrylamide;
N-(4-(4-amino-5-(3-fluoro-4-((6-methylpyridin-2-yl)oxy)phenyl)pyrazolo[5,1-f][1,2,4]triazin-6-yl)phenyl)methacrylamide;
N-(4-(4-amino-5-(3-fluoro-4-((6-methylpyridin-2-yl)oxy)phenyl)pyrazolo[5,1-f][1,2,4]triazin-6-yl)phenyl)but-2-ynamide;
N-(4-(4-amino-5-(3-fluoro-4-((6-methylpyridin-2-yl)oxy)phenyl)pyrazolo[5,1-f][1,2,4]triazin-6-yl)phenyl)propiolamide;
N-(4-(4-amino-5-(3-fluoro-4-((6-methylpyridin-2-yl)oxy)phenyl)pyrazolo[5,1-f][1,2,4]triazin-6-yl)phenyl)ethenesulfonamide;
(E)-N-(4-(4-amino-5-(3-fluoro-4-((6-methylpyridin-2-yl)oxy)phenyl)pyrazolo[5,1-f][1,2,4]triazin-6-yl)phenyl)-4-(dimethylamino)but-2-enamide;
1-(4-(4-amino-5-(3-fluoro-4-((6-methylpyridin-2-yl)oxy)phenyl)pyrazolo[5,1-f][1,2,4]triazin-6-yl)-3,6-dihydropyridin-1(2H)-yl)prop-2-en-1-one;
(E)-N-(4-(4-amino-5-(3-fluoro-4-((6-methylpyridin-2-yl)oxy)phenyl)pyrazolo[5,1-f][1,2,4]triazin-6-yl)phenyl)but-2-enamide 4-(4-amino-6-(4-(2-fluoroacrylamido)-2-methoxyphenyl)pyrazolo[5,1-f][1,2,4]triazin-5-yl)-2-methoxy-N-(1-(trifluoromethyl)cyclopropyl)benzamide;
4-(4-amino-6-(4-(2-fluoroacrylamido)-2-methoxyphenyl)pyrazolo[5,1-f][1,2,4]triazin-5-yl)-N-(3-fluorobicyclo[1.1.1]pentan-1-yl)-2-methoxybenzamide;
N-(4-(4-amino-5-(3-fluoro-4-(methyl(4-methylpyrimidin-2-yl)amino)phenyl)pyrazolo[5,1-f][1,2,4]triazin-6-yl)phenyl)acrylamide;
N-(4-(4-amino-5-(4-benzoyl-3-methoxyphenyl)pyrazolo[5,1-f][1,2,4]triazin-6-yl)phenyl) acrylamide;
N-(4-(4-amino-5-(4-(4,4-difluorocyclohexane-1-carbonyl)-3-methoxyphenyl)pyrazolo[5,1-f][1,2,4]triazin-6-yl)phenyl)acrylamide;
1-(4-(4-amino-5-(3-fluoro-4-((6-methylpyridin-2-yl)oxy)phenyl)pyrazolo[5,1-f][1,2,4]triazin-6-yl)piperidin-1-yl)prop-2-en-1-one;
1-(4-(4-(4-amino-5-(3-fluoro-4-((6-methylpyridin-2-yl)oxy)phenyl)pyrazolo[5,1-f][1,2,4]triazin-6-yl)-1H-pyrazol-1-yl)piperidin-1-yl)prop-2-en-1-one;
N-(4-(4-amino-5-(3-fluoro-4-((6-methylpyridin-2-yl)oxy)phenyl)pyrazolo[5,1-f][1,2,4]triazin-6-yl)phenyl)-2-((dimethylamino)methyl)acrylamide;
N-(4-(4-amino-5-(3-fluoro-4-((6-methylpyridin-2-yl)oxy)phenyl)pyrazolo[5,1-f][1,2,4]triazin-6-yl)phenyl)-2-chloroacetamide;
N-(4-(4-amino-5-(3-fluoro-4-((6-methylpyridin-2-yl)oxy)phenyl)pyrazolo[5,1-f][1,2,4]triazin-6-yl)phenyl)-2-chloroacrylamide;
(Z)-4-((4-(4-amino-5-(3-fluoro-4-((6-methylpyridin-2-yl)oxy)phenyl)pyrazolo[5,1-f][1,2,4]triazin-6-yl)phenyl)amino)-4-oxobut-2-enoic acid;
4-(4-amino-6-(4-(2-fluoroacrylamido)-2-methoxyphenyl)pyrazolo[5,1-f][1,2,4]triazin-5-yl)-N-(bicyclo[1.1.1]pentan-1-yl)-2-methoxybenzamide;
4-(4-amino-6-(2-fluoro-4-(2-fluoroacrylamido)phenyl)pyrazolo[5,1-f][1,2,4]triazin-5-yl)-N-(3-fluorobicyclo[1.1.1]pentan-1-yl)-2-methoxybenzamide;
N-(4-(4-amino-5-(4-((4-aminopyrimidin-2-yl)oxy)-3-fluorophenyl)pyrazolo[5,1-f][1,2,4]triazin-6-yl)phenyl)acrylamide;
4-(4-amino-6-(2-fluoro-4-methacrylamidophenyl)pyrazolo[5,1-f][1,2,4]triazin-5-yl)-2-methoxy-N-(1-(trifluoromethyl)cyclopropyl)benzamide;
N-(4-(4-amino-7-cyano-3-(3-fluoro-4-((6-methylpyridin-2-yl)oxy)phenyl)-1-methyl-1H-pyrrolo[3,2-c]pyridin-2-yl)phenyl)acrylamide;
N-(4-(4-amino-5-(4-(cyclohexanecarbonyl)-3-methoxyphenyl)pyrazolo[5,1-f][1,2,4]triazin-6-yl)phenyl)acrylamide;
4-(4-amino-6-(4-(2-fluoroacrylamido)-2-(trifluoromethyl)phenyl)pyrazolo[5,1-f][1,2,4]triazin-5-yl)-2-methoxy-N-(1-(trifluoromethyl)cyclopropyl)benzamide;
N-(4-(4-amino-5-(3-fluoro-4-((4-(methylamino)pyrimidin-2-yl)oxy)phenyl)pyrazolo[5,1-f][1,2,4]triazin-6-yl)phenyl)acrylamide;
N-(4-(4-amino-5-(3-fluoro-4-((6-methylpyridin-2-yl)oxy)phenyl)pyrazolo[5,1-f][1,2,4]triazin-6-yl)-3-fluorophenyl)methacrylamide;
N-(4-(4-amino-7-cyano-3-(4-((4-(difluoromethyl)pyrimidin-2-yl)oxy)-3-fluorophenyl)-1-methyl-1H-pyrrolo[3,2-c]pyridin-2-yl)phenyl)acrylamide;
N-(4-(4-amino-7-cyano-3-(3-fluoro-4-((5-fluoro-4-methylpyrimidin-2-yl)oxy)phenyl)-1-methyl-1H-pyrrolo[3,2-c]pyridin-2-yl)phenyl)acrylamide;
N-(4-(4-amino-7-cyano-1-methyl-3-(4-((6-methylpyridin-2-yl)oxy)phenyl)-1H-pyrrolo[3,2-c]pyridin-2-yl)phenyl)acrylamide;
N-(4-(4-amino-5-(3-fluoro-4-((6-methylpyridin-2-yl)oxy)phenyl)pyrazolo[5,1-f][1,2,4]triazin-6-yl)-3-fluorophenyl)-2-fluoroacrylamide;
N-(4-(4-amino-5-(3-fluoro-4-((6-methylpyridin-2-yl)oxy)phenyl)pyrazolo[5,1-f][1,2,4]triazin-6-yl)-3-methylphenyl)-2-fluoroacrylamide;
N-(4-(4-amino-7-cyano-3-(3-fluoro-4-((1-methyl-1H-pyrazol-3-yl)oxy)phenyl)-1-methyl-1H-pyrrolo[3,2-c]pyridin-2-yl)phenyl)acrylamide;
N-(4-(4-amino-5-(2-(1-methylcyclopropyl)benzo[d]thiazol-6-yl)pyrazolo[5,1-f][1,2,4]triazin-6-yl)phenyl)acrylamide;
N-(4-(4-amino-5-(3-methoxy-4-((1-methylcyclohexyl)oxy)phenyl)pyrazolo[5,1-f][1,2,4]triazin-6-yl)phenyl)acrylamide;
N-(4-(4-amino-7-cyano-3-(3-fluoro-4-(pyrimidin-2-yloxy)phenyl)-1-methyl-1H-pyrrolo[3,2-c]pyridin-2-yl)phenyl)acrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)phenyl)acrylamide;
4-(4-amino-6-(2-chloro-4-(2-fluoroacrylamido)phenyl)pyrazolo[5,1-f][1,2,4]triazin-5-yl)-2-methoxy-N-(1-(trifluoromethyl)cyclopropyl)benzamide;
4-(4-amino-6-(4-(2-fluoroacrylamido)-2-methylphenyl)pyrazolo[5,1-f][1,2,4]triazin-5-yl)-2-methoxy-N-(1-(trifluoromethyl)cyclopropyl)benzamide;
4-(4-amino-6-(2-chloro-4-(2-fluoroacrylamido)phenyl)pyrazolo[5,1-f][1,2,4]triazin-5-yl)-2-methoxy-N-(1-(trifl4-(4-amino-6-(2-chloro-4-(2-fluoroacrylamido)phenyl)pyrazolo[5,1-f][1,2,4]triazin-5-yl)-2-methoxy-N-(1-(trifluoromethyl)cyclopropyl)benzamide;
N-(4-(4-amino-5-(3-fluoro-4-((6-methylpyridin-2-yl)oxy)phenyl)pyrazolo[5,1-f][1,2,4]triazin-6-yl)-3-chlorophenyl)-2-fluoroacrylamide;
N-(4-(4-amino-5-(3-fluoro-4-((4-methylpyrimidin-2-yl)oxy)phenyl)pyrazolo[5,1-f][1,2,4]triazin-6-yl)-3-methoxyphenyl)methacrylamide;
N-(4-(4-amino-5-(3-fluoro-4-((4-methylpyrimidin-2-yl)oxy)phenyl)pyrazolo[5,1-f][1,2,4]triazin-6-yl)-3-methylphenyl)methacrylamide;
N-(4-(4-amino-5-(3-fluoro-4-((4-methylpyrimidin-2-yl)oxy)phenyl)pyrazolo[5,1-f][1,2,4]triazin-6-yl)-3-chlorophenyl)methacrylamide;
N-(4-(4-amino-5-(3-fluoro-4-((1-methyl-1H-pyrazol-3-yl)oxy)phenyl)pyrazolo[5,1-f][1,2,4]triazin-6-yl)phenyl)methacrylamide;
N-(4-(4-amino-5-(3-fluoro-4-((1-methyl-1H-pyrazol-3-yl)oxy)phenyl)pyrazolo[5,1-f][1,2,4]triazin-6-yl)-3-fluorophenyl)methacrylamide;
N-(4-(4-amino-5-(3-fluoro-4-((1-methyl-1H-pyrazol-3-yl)oxy)phenyl)pyrazolo[5,1-f][1,2,4]triazin-6-yl)phenyl)but-2-ynamide;
N-(4-(4-amino-5-(3-fluoro-4-((1-methyl-1H-pyrazol-3-yl)oxy)phenyl)pyrazolo[5,1-f][1,2,4]triazin-6-yl)phenyl)propiolamide;
N-(4-(4-amino-5-(3-methoxy-4-(1-methyl-1H-imidazole-2-carbonyl)phenyl) pyrazolo[5,1-f][1,2,4]triazin-6-yl)phenyl)acrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)phenyl)acrylamide;
N-(4-(4-amino-7-cyano-3-(4-((5-fluoropyrimidin-2-yl)oxy)phenyl)-1-methyl-1H-pyrrolo[3,2-c]pyridin-2-yl)-3-fluorophenyl)methacrylamide;
N-(4-(4-amino-5-(3-fluoro-4-((6-methylpyridin-2-yl)oxy)phenyl)pyrazolo[5,1-f][1,2,4]triazin-6-yl)-3-methoxyphenyl)-2-fluoroacrylamide;
N-(4-(4-amino-7-cyano-3-(4-((4-(difluoromethyl)pyrimidin-2-yl)oxy)-3-fluorophenyl)-1-methyl-1H-pyrrolo[3,2-c]pyridin-2-yl)-3-fluorophenyl)methacrylamide;
N-(4-(4-amino-7-cyano-3-(3-fluoro-4-((5-fluoro-4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-(pyrimidin-2-yloxy)phenyl)-1-methyl-1H-pyrrolo[3,2-c]pyridin-2-yl)-3-fluorophenyl)methacrylamide;
N-(4-(4-amino-7-cyano-3-(4-((4-ethylpyrimidin-2-yl)oxy)-3-fluorophenyl)-1-methyl-1H-pyrrolo[3,2-c]pyridin-2-yl)-3-fluorophenyl)methacrylamide;
N-(4-(4-amino-7-cyano-3-(3-fluoro-4-((6-methylpyridin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-(pyridin-2-yloxy)phenyl)-1-methyl-1H-pyrrolo[3,2-c]pyridin-2-yl)-3-fluorophenyl)methacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-fluorophenyl)methacrylamide;
N-(4-(4-amino-7-cyano-1-methyl-3-(4-((6-methylpyridin-2-yl)oxy)phenyl)-1H-pyrrolo[3,2-c]pyridin-2-yl)-3-fluorophenyl)methacrylamide;
N-(4-(4-amino-7-cyano-1-methyl-3-(4-(pyrimidin-2-yloxy)phenyl)-1H-pyrrolo[3,2-c]pyridin-2-yl)-3-fluorophenyl)methacrylamide;
4-(4-amino-7-cyano-2-(2-fluoro-4-methacrylamidophenyl)-1-methyl-1H-pyrrolo[3,2-c]pyridin-3-yl)-2-methoxy-N-(2,2,2-trifluoroethyl)benzamide;
N-(4-(4-amino-3-(3-chloro-4-((4-methylpyrimidin-2-yl)oxy)phenyl)-7-cyano-1-methyl-1H-pyrrolo[3,2-c]pyridin-2-yl)-3-fluorophenyl)methacrylamide;
N-(4-(4-amino-7-cyano-1-methyl-3-(3-methyl-4-((4-methylpyrimidin-2-yl)oxy)phenyl)-1H-pyrrolo[3,2-c]pyridin-2-yl)-3-fluorophenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-chlorophenyl)methacrylamide;
N-(4-(4-amino-7-cyano-3-(3,5-difluoro-4-((4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(4-((4,5-dimethylpyrimidin-2-yl)oxy)-3-fluorophenyl)-1-methyl-1H-pyrrolo[3,2-c]pyridin-2-yl)-3-fluorophenyl)methacrylamide;
N-(4-(4-amino-7-cyano-3-(3-fluoro-4-((5-fluoropyrimidin-2-yl)oxy)phenyl)-1-methyl-1H-pyrrolo[3,2-c]pyridin-2-yl)phenyl)acrylamide;
N-(4-(4-amino-7-cyano-3-(3-fluoro-4-(pyridin-2-yloxy)phenyl)-1-methyl-1H-pyrrolo[3,2-c]pyridin-2-yl)phenyl)acrylamide;
N-(4-(4-amino-7-cyano-3-(3-fluoro-4-((5-fluoropyridin-2-yl)oxy)phenyl)-1-methyl-1H-pyrrolo[3,2-c]pyridin-2-yl)phenyl)acrylamide;
N-(4-(4-amino-7-cyano-3-(4-((4,5-dimethylpyrimidin-2-yl)oxy)-3-fluorophenyl)-1-methyl-1H-pyrrolo[3,2-c]pyridin-2-yl)phenyl)acrylamide;
N-(4-(4-amino-7-cyano-3-(4-(cyclohexyloxy)phenyl)-1-methyl-1H-pyrrolo[3,2-c]pyridin-2-yl)-3-fluorophenyl)methacrylamide;
4-(4-amino-7-cyano-2-(2-fluoro-4-methacrylamidophenyl)-1-methyl-1H-pyrrolo[3,2-c]pyridin-3-yl)-2-methoxy-N-(1-(trifluoromethyl)cyclopropyl)benzamide;
N-(4-(4-amino-7-cyano-1-methyl-3-(4-(pyridin-2-yloxy)phenyl)-1H-pyrrolo[3,2-c]pyridin-2-yl)-3-fluorophenyl)methacrylamide;
N-(4-(4-amino-7-cyano-1-methyl-3-(4-(pyridin-2-yloxy)phenyl)-1H-pyrrolo[3,2-c]pyridin-2-yl)phenyl)acrylamide;
N-(4-(4-amino-7-cyano-3-(3-fluoro-4-((5-fluoropyrimidin-2-yl)oxy)phenyl)-1-methyl-1H-pyrrolo[3,2-c]pyridin-2-yl)-3-fluorophenyl)methacrylamide;
N-(4-(4-amino-3-(3-chloro-4-((5-fluoro-4-methylpyrimidin-2-yl)oxy)phenyl)-7-cyano-1-methyl-1H-pyrrolo[3,2-c]pyridin-2-yl)phenyl)acrylamide;
N-(4-(4-amino-3-(4-((6-chloropyridin-2-yl)oxy)-3-fluorophenyl)-7-cyano-1-methyl-1H-pyrrolo[3,2-c]pyridin-2-yl)-3-fluorophenyl)methacrylamide;
N-(4-(4-amino-7-cyano-3-(2,5-difluoro-4-((4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(2,3-difluoro-4-((4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-2-fluoro-3-methylphenyl)methacrylamide;
N-(4-(4-amino-7-cyano-3-(2-fluoro-4-((4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-((5-fluoropyridin-2-yl)oxy)phenyl)-1-methyl-1H-pyrrolo[3,2-c]pyridin-2-yl)-3-fluorophenyl)methacrylamide;
N-(4-(4-amino-7-cyano-3-(4-((5-fluoropyridin-2-yl)oxy)phenyl)-1-methyl-1H-pyrrolo[3,2-c]pyridin-2-yl)phenyl)acrylamide;
N-(4-(4-amino-7-cyano-3-(4-((5-fluoro-4-methylpyrimidin-2-yl)oxy)-3-methylphenyl)-1-methyl-1H-pyrrolo[3,2-c]pyridin-2-yl)phenyl)acrylamide;
N-(4-(4-amino-7-cyano-3-(3,5-difluoro-4-((5-fluoro-4-methylpyrimidin-2-yl)oxy)phenyl)-1-methyl-1H-pyrrolo[3,2-c]pyridin-2-yl)phenyl)acrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-fluorophenyl)acrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-fluorophenyl)methacrylamide;
N-(4-(4-amino-7-cyano-3-(4-((5-fluoropyridin-2-yl)oxy)phenyl)-1-methyl-1H-pyrrolo[3,2-c]pyridin-2-yl)-3-fluorophenyl)methacrylamide;
N-(4-(4-amino-7-cyano-3-(2,3-difluoro-4-((5-fluoro-4-methylpyrimidin-2-yl)oxy)phenyl)-1-methyl-1H-pyrrolo[3,2-c]pyridin-2-yl)phenyl)acrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-methylphenyl)acrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-chlorophenyl)acrylamide;
N-(4-(4-amino-7-cyano-3-(4-(cyclohexyloxy)phenyl)-1-methyl-1H-pyrrolo[3,2-c]pyridin-2-yl)phenyl)acrylamide;
N-(4-(4-amino-7-cyano-3-(4-(cyclohexanecarbonyl)-3-fluorophenyl)-1-methyl-1H-pyrrolo[3,2-c]pyridin-2-yl)phenyl)acrylamide;
N-(4-(4-amino-7-cyano-3-(3-fluoro-4-(thiazol-2-yloxy)phenyl)-1-methyl-1H-pyrrolo[3,2-c]pyridin-2-yl)phenyl)acrylamide;
N-(4-(4-amino-7-cyano-3-(3-fluoro-4-((4-methylthiazol-2-yl)oxy)phenyl)-1-methyl-1H-pyrrolo[3,2-c]pyridin-2-yl)phenyl)acrylamide;
N-(4-(4-amino-7-cyano-3-(3-fluoro-4-((4-(trifluoromethyl)pyrimidin-2-yl)oxy)phenyl)-1-methyl-1H-pyrrolo[3,2-c]pyridin-2-yl)phenyl)acrylamide;
N-(4-(4-amino-7-cyano-3-(3-fluoro-4-((4-(trifluoromethyl)pyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-2,3-difluorophenyl)methacrylamide;
N-(4-(4-amino-7-cyano-3-(3-fluoro-4-((5-fluoro-6-methylpyridin-2-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-((5-fluoro-6-methylpyridin-2-yl)oxy)phenyl)-1-methyl-1H-pyrrolo[3,2-c]pyridin-2-yl)phenyl)acrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-2,3-difluorophenyl)acrylamide;
N-(4-(4-amino-7-cyano-3-(4-(cyclohexyloxy)-3-fluorophenyl)-1-methyl-1H-pyrrolo[3,2-c]pyridin-2-yl)-3-fluorophenyl)methacrylamide;
N-(4-(4-amino-7-cyano-3-(4-((1,5-dimethyl-1H-pyrazol-3-yl)oxy)-3-fluorophenyl)-1-methyl-1H-pyrrolo[3,2-c]pyridin-2-yl)phenyl)acrylamide;
N-(4-(4-amino-7-cyano-3-(3-fluoro-4-((1-methyl-5-(trifluoromethyl)-1H-pyrazol-3-yl)oxy)phenyl)-1-methyl-1H-pyrrolo[3,2-c]pyridin-2-yl)phenyl)acrylamide;
N-(4-(4-amino-7-cyano-1-methyl-3-(4-((1-methyl-5-(trifluoromethyl)-1H-pyrazol-3-yl)oxy)phenyl)-1H-pyrrolo[3,2-c]pyridin-2-yl)phenyl)acrylamide;
N-(4-(4-amino-7-cyano-1-methyl-3-(4-((4-(trifluoromethyl)pyrimidin-2-yl)oxy)phenyl)-1H-pyrrolo[3,2-c]pyridin-2-yl)phenyl)acrylamide;
N-(4-(4-amino-7-cyano-1-methyl-3-(4-((4-(trifluoromethyl)pyrimidin-2-yl)oxy)phenyl)-1H-pyrrolo[3,2-c]pyridin-2-yl)-3-fluorophenyl)methacrylamide;
N-(4-(4-amino-7-cyano-3-(4-((4,6-dimethylpyrimidin-2-yl)oxy)phenyl)-1-methyl-1H-pyrrolo[3,2-c]pyridin-2-yl)phenyl)acrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-chlorophenyl)methacrylamide;
N-(4-(4-amino-7-cyano-1-methyl-3-(4-((1-methyl-1H-pyrazol-5-yl)oxy)phenyl)-1H-pyrrolo[3,2-c]pyridin-2-yl)phenyl)acrylamide;
N-(4-(4-amino-7-cyano-1-methyl-3-(3-methyl-4-((4-methylpyrimidin-2-yl)oxy)phenyl)-1H-pyrrolo[3,2-c]pyridin-2-yl)-3-chlorophenyl)methacrylamide;
N-(4-(4-amino-7-cyano-3-(4-((1-cyclopropyl-1H-pyrazol-3-yl)oxy)-3-fluorophenyl)-1-methyl-1H-pyrrolo[3,2-c]pyridin-2-yl)phenyl)acrylamide;
N-(4-(4-amino-3-(3-chloro-4-((4-methylpyrimidin-2-yl)oxy)phenyl)-7-cyano-1-methyl-1H-pyrrolo[3,2-c]pyridin-2-yl)-3-chlorophenyl)methacrylamide;
N-(4-(4-amino-7-cyano-1-methyl-3-(4-((6-methylpyridin-2-yl)oxy)phenyl)-1H-pyrrolo[3,2-c]pyridin-2-yl)-3-chlorophenyl)methacrylamide;
4-(3-acrylamido-12-amino-9-cyano-6,7-dihydrobenzo[f]pyrido[3′,4′:4,5]pyrrolo[1,2-d][1,4]oxazepin-13-yl)-N-(3,3-difluorocyclobutyl)-2-methoxybenzamide;
N-(4-(4-amino-3-(3-chloro-4-((4-methylpyrimidin-2-yl)oxy)phenyl)-7-cyano-1-methyl-1H-pyrrolo[3,2-c]pyridin-2-yl)-3-methylphenyl)methacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-chlorophenyl)methacrylamide;
N-(4-(4-amino-7-cyano-3-(3-fluoro-4-((6-methylpyridin-2-yl)oxy)phenyl)-1-methyl-1H-pyrrolo[3,2-c]pyridin-2-yl)-3-chlorophenyl)methacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-fluorophenyl)acrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-fluorophenyl)acrylamide;
N-(4-(4-amino-7-cyano-3-(4-(cyclohexyloxy)phenyl)-1-methyl-1H-pyrrolo[3,2-c]pyridin-2-yl)-3-chlorophenyl)methacrylamide;
N-(4-(4-amino-5-(4-(cyclohexyloxy)-3-fluorophenyl)pyrazolo[5,1-f][1,2,4]triazin-6-yl)phenyl)-2-fluoroacrylamide;
N-(4-(4-amino-5-(4-(cyclohexyloxy)-3-fluorophenyl)pyrazolo[5,1-f][1,2,4]triazin-6-yl)phenyl)acrylamide;
N-(4-(4-amino-7-cyano-3-(4-(cyclohexyloxy)-3-fluorophenyl)-1-methyl-1H-pyrrolo[3,2-c]pyridin-2-yl)phenyl)acrylamide;
N-(4-(4-amino-5-(3-methoxy-4-((4-methylpyrimidin-2-yl)oxy)phenyl)pyrazolo[5,1-f][1,2,4]triazin-6-yl)phenyl)acrylamide;
4-(2-(4-acrylamidophenyl)-4-amino-7-cyano-1-methyl-1H-pyrrolo[3,2-c]pyridin-3-yl)-2-methoxy-N-(2,2,2-trifluoroethyl)benzamide;
4-(4-amino-6-(4-(2-fluoroacrylamido)phenyl) pyrazolo[5,1-f][1,2,4]triazin-5-yl)-2-methoxy-N-(2,2,2-trifluoroethyl)benzamide;
4-(4-amino-7-cyano-2-(4-(2-fluoroacrylamido)phenyl)-1-methyl-1H-pyrrolo[3,2-c]pyridine-3-yl)-2-methoxy-N-(2,2,2-trifluoroethyl)benzamide;
4-(4-amino-6-(2-fluoro-4-(2-fluoroacrylamido)phenyl)pyrazolo[5,1-f][1,2,4]triazin-5-yl)-2-methoxy-N-(2,2,2-trifluoroethyl)benzamide;
4-(4-amino-6-(4-(2-fluoroacrylamido)phenyl)pyrazolo[5,1-f][1,2,4]triazin-5-yl)-N-(3,3-difluorocyclobutyl)benzamide;
N-(4-(6-(4-acrylamidophenyl)-4-aminopyrazolo[5,1-f][1,2,4]triazin-5-yl)-2-methoxyphenyl)-2,2-difluorocyclopropane-1-carboxamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)phenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)phenyl)-2-fluoroacrylamide;
N-(4-(4-amino-5-(3-fluoro-4-((4-methylpyrimidin-2-yl)oxy)phenyl)pyrazolo[5,1-f][1,2,4]triazin-6-yl)phenyl)methacrylamide;
4-(2-(4-acrylamidophenyl)-4-amino-7-cyano-1-(methyl-d3)-1H-pyrrolo[3,2-c]pyridin-3-yl)-2-methoxy-N-(2,2,2-trifluoroethyl)benzamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-fluorophenyl)methacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-chlorophenyl)methacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-methylphenyl)methacrylamide;
N-(4-(4-amino-7-cyano-3-(3-fluoro-4-((1-methyl-1H-pyrazol-3-yl)oxy)phenyl)-1-methyl-1H-pyrrolo[3,2-c]pyridin-2-yl)-3-fluorophenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-methylphenyl)methacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-chlorophenyl)methacrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)phenyl)acrylamide;
4-(3-acrylamido-12-amino-9-cyano-6,7-dihydrobenzo[f]pyrido[3′,4′:4,5]pyrrolo[1,2-d][1,4]oxazepin-13-yl)-N-(3,3-difluorocyclobutyl)-2-methoxybenzamide;
N-(4-(4-amino-7-cyano-3-(3-fluoro-4-((6-methylpyridin-2-yl)oxy)phenyl)-1-methyl-1H-pyrrolo[3,2-c]pyridin-2-yl)-3-methylphenyl)methacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-methylphenyl)methacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-methylphenyl)methacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-methylphenyl)methacrylamide;
N-(4-(4-amino-7-cyano-1-methyl-3-(3-methyl-4-((4-methylpyrimidin-2-yl)oxy)phenyl)-1H-pyrrolo[3,2-c]pyridin-2-yl)-3-methylphenyl)methacrylamide;
N-(4-(4-amino-7-cyano-1-methyl-3-(4-((6-methylpyridin-2-yl)oxy)phenyl)-1H-pyrrolo[3,2-c]pyridin-2-yl)-3-methylphenyl)methacrylamide;
N-(4-(4-amino-7-cyano-3-(4-((4-(difluoromethyl)pyrimidin-2-yl)oxy)-3-fluorophenyl)-1-methyl-1H-pyrrolo[3,2-c]pyridin-2-yl)-3-chlorophenyl)methacrylamide;
N-(4-(4-amino-7-cyano-3-(3-fluoro-4-((5-fluoro-4-methylpyrimidin-2-yl)oxy)phenyl)-1-methyl-1H-pyrrolo[3,2-c]pyridin-2-yl)-3-chlorophenyl)methacrylamide;
N-(4-(4-amino-7-cyano-3-(3-fluoro-4-((4-(trifluoromethyl)pyrimidin-2-yl)oxy)phenyl)-1-methyl-1H-pyrrolo[3,2-c]pyridin-2-yl)-3-chlorophenyl)methacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-methylphenyl)-2-fluoroacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-methylphenyl)-2-fluoroacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-methylphenyl)-2-fluoroacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-methylphenyl)-2-fluoroacrylamide;
N-(4-(4-amino-7-cyano-1-methyl-3-(4-((6-methylpyridin-2-yl)oxy)phenyl)-1H-pyrrolo[3,2-c]pyridin-2-yl)-3-methylphenyl)-2-fluoroacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-chlorophenyl)-2-fluoroacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-chlorophenyl)-2-fluoroacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-chlorophenyl)-2-fluoroacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-chlorophenyl)-2-fluoroacrylamide;
N-(4-(4-amino-7-cyano-1-methyl-3-(4-((6-methylpyridin-2-yl)oxy)phenyl)-1H-pyrrolo[3,2-c]pyridin-2-yl)-3-chlorophenyl)-2-fluoroacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-fluorophenyl)-2-fluoroacrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)-3-fluorophenyl)-2-fluoroacrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-fluorophenyl)-2-fluoroacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-fluorophenyl)-2-fluoroacrylamide;
N-(4-(4-amino-7-cyano-1-methyl-3-(4-((6-methylpyridin-2-yl)oxy)phenyl)-1H-pyrrolo[3,2-c]pyridin-2-yl)-3-fluorophenyl)-2-fluoroacrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-methylphenyl)acrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-methylphenyl)acrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-methylphenyl)acrylamide;
N-(4-(4-amino-7-cyano-1-methyl-3-(4-((6-methylpyridin-2-yl)oxy)phenyl)-1H-pyrrolo[3,2-c]pyridin-2-yl)-3-methylphenyl)acrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-chlorophenyl)acrylamide;
N-(4-(4-amino-7-cyano-3-(4-((4-(difluoromethyl)pyrimidin-2-yl)oxy)phenyl)-1-methyl-1H-pyrrolo[3,2-c]pyridin-2-yl)-3-chlorophenyl)acrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-chlorophenyl)acrylamide;
N-(4-(4-amino-7-cyano-1-methyl-3-(4-((6-methylpyridin-2-yl)oxy)phenyl)-1H-pyrrolo[3,2-c]pyridin-2-yl)-3-chlorophenyl)acrylamide;
N-(4-(4-amino-7-cyano-1-methyl-3-(4-((1-methyl-1H-pyrazol-3-yl)oxy)phenyl)-1H-pyrrolo[3,2-c]pyridin-2-yl)-3-fluorophenyl)acrylamide;
N-(4-(4-amino-7-cyano-1-methyl-3-(4-((6-methylpyridin-2-yl)oxy)phenyl)-1H-pyrrolo[3,2-c]pyridin-2-yl)-3-fluorophenyl)acrylamide;
N-(4-(4-amino-7-cyano-3-(4-((5-fluoro-4-methylpyrimidin-2-yl)oxy)phenyl)-1-methyl-1H-pyrrolo[3,2-c]pyridin-2-yl)phenyl)-2-methylprop-2-enethioamide;
N-(4-(4-amino-7-cyano-3-(4-((4-methylpyrimidin-2-yl)oxy)phenyl)-1H-pyrrolo[3,2-c]pyridin-2-yl)-3-chlorophenyl)methacrylamide;
N-(4-(4-amino-7-cyano-1-ethyl-3-(4-((4-methylpyrimidin-2-yl)oxy)phenyl)-1H-pyrrolo[3,2-c]pyridin-2-yl)-3-chlorophenyl)methacrylamide;
N-(4-(4-amino-7-cyano-1-(2-fluoroethyl)-3-(4-((4-methylpyrimidin-2-yl)oxy)phenyl)-1H-pyrrolo[3,2-c]pyridin-2-yl)-3-chlorophenyl)methacrylamide;
N-(4-(4-amino-7-cyano-3-(4-((4-methylpyrimidin-2-yl)oxy)phenyl)-1-(2,2,2-trifluoroethyl)-1H-pyrrolo[3,2-c]pyridin-2-yl)-3-chlorophenyl)methacrylamide;
N-(4-(4-amino-7-cyano-3-(4-((4-(difluoromethyl)-5-fluoropyrimidin-2-yl)oxy)phenyl)-1-methyl-1H-pyrrolo[3,2-c]pyridin-2-yl)phenyl)acrylamide;
N-(4-(4-amino-7-cyano-3-(4-((4-(difluoromethyl)-5-fluoropyrimidin-2-yl)oxy)-3-fluorophenyl)-1-methyl-1H-pyrrolo[3,2-c]pyridin-2-yl)phenyl)acrylamide;
N-(4-(4-amino-3-(3-chloro-5-fluoro-4-((5-fluoro-4-methylpyrimidin-2-yl)oxy)phenyl)-7-cyano-1-methyl-1H-pyrrolo[3,2-c]pyridin-2-yl)phenyl)acrylamide;
N-(4-(4-amino-7-cyano-3-(3-fluoro-4-((1-(2,2,2-trifluoroethyl)-1H-pyrazol-3-yl)oxy)phenyl)-1-methyl-1H-pyrrolo[3,2-c]pyridin-2-yl)phenyl)acrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3,5-difluorophenyl)methacrylamide;
N-(4-(4-amino-7-cyano-3-(3-fluoro-4-((4-methylpyrimidin-2-yl)oxy)phenyl)-1-methyl-1H-pyrrolo[3,2-c]pyridin-2-yl)-3-fluoro-5-methylphenyl)methacrylamide;
N-(4-(4-amino-7-cyano-1-methyl-3-(4-((4-methylpyrimidin-2-yl)oxy)phenyl)-1H-pyrrolo[3,2-c]pyridin-2-yl)-3-(trifluoromethyl)phenyl)methacrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)phenyl)acrylamide;
N-(4-(4-amino-3-(4-((5-chloro-4-methylpyrimidin-2-yl)oxy)-3,5-difluorophenyl)-7-cyano-1-methyl-1H-pyrrolo[3,2-c]pyridin-2-yl)phenyl)acrylamide;
N-(4-(4-amino-3-(4-((5-chloro-4-(trifluoromethyl)pyrimidin-2-yl)oxy)phenyl)-7-cyano-1-methyl-1H-pyrrolo[3,2-c]pyridin-2-yl)phenyl)acrylamide;
N-(4-(4-amino-3-(4-((5-chloro-4-(trifluoromethyl)pyrimidin-2-yl)oxy)-3-fluorophenyl)-7-cyano-1-methyl-1H-pyrrolo[3,2-c]pyridin-2-yl)phenyl)acrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-chlorophenyl)methacrylamide;
N-(4-(4-amino-7-cyano-1-methyl-3-(4-((4-(trifluoromethyl)pyrimidin-2-yl)oxy)phenyl)-1H-pyrrolo[3,2-c]pyridin-2-yl)-3-chlorophenyl)methacrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-methylphenyl)methacrylamide;
N-(4-(4-amino-7-cyano-1-methyl-3-(4-((4-(trifluoromethyl)pyrimidin-2-yl)oxy)phenyl)-1H-pyrrolo[3,2-c]pyridin-2-yl)-3-methylphenyl)methacrylamide;
N-(4-(4-amino-7-cyano-3-(3-fluoro-4-((4-(trifluoromethyl)pyrimidin-2-yl)oxy)phenyl)-1-methyl-1H-pyrrolo[3,2-c]pyridin-2-yl)-3-methylphenyl)methacrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)phenyl)but-2-ynamide;
N-(4-(4-amino-3-(4-((5-chloro-4-(difluoromethyl)pyrimidin-2-yl)oxy)phenyl)-7-cyano-1-methyl-1H-pyrrolo[3,2-c]pyridin-2-yl)phenyl)acrylamide;
N-(4-(4-amino-3-(4-((5-chloro-4-(difluoromethyl)pyrimidin-2-yl)oxy)-3-fluorophenyl)-7-cyano-1-methyl-1H-pyrrolo[3,2-c]pyridin-2-yl)phenyl)acrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)phenyl)-2-fluoroacrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)phenyl)-2-fluoroacrylamide;
N-(4-(4-amino-3-(4-((5-chloro-6-methylpyridin-2-yl)oxy)phenyl)-7-cyano-1-methyl-1H-pyrrolo[3,2-c]pyridin-2-yl)phenyl)acrylamide;
N-(4-(4-amino-3-(4-((5-chloro-6-methylpyridin-2-yl)oxy)-3-fluorophenyl)-7-cyano-1-methyl-1H-pyrrolo[3,2-c]pyridin-2-yl)phenyl)acrylamide;
N-(4-(4-amino-3-(3-chloro-4-((5-chloro-4-methylpyrimidin-2-yl)oxy)phenyl)-7-cyano-1-methyl-1H-pyrrolo[3,2-c]pyridin-2-yl)phenyl)acrylamide;
N-(4-(4-amino-3-(4-((5-chloro-4-methylpyrimidin-2-yl)oxy)-3-methylphenyl)-7-cyano-1-methyl-1H-pyrrolo[3,2-c]pyridin-2-yl)phenyl)acrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-chlorophenyl)methacrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-methylphenyl)acrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-methylphenyl)acrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-methylphenyl)-2-fluoroacrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-methylphenyl)-2-fluoroacrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-chlorophenyl)acrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-chlorophenyl)acrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-chlorophenyl)-2-fluoroacrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-chlorophenyl)-2-fluoroacrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-fluorophenyl)acrylamide;
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-fluorophenyl)acrylamide;
N-(4-(4-amino-3-(4-((5-chloro-4-methylpyrimidin-2-yl)oxy)-3-fluorophenyl)-7-cyano-1-methyl-1H-pyrrolo[3,2-c]pyridin-2-yl)-3-fluorophenyl)-2-fluoroacrylamide; or
N-(4-(4-amino-3-(4-((5-chloro-4-methylpyrimidin-2-yl)oxy)phenyl)-7-cyano-1-methyl-1H-pyrrolo[3,2-c]pyridin-2-yl)-3-fluorophenyl)-2-fluoroacrylamide.

76. A pharmaceutical composition, comprising a compound of any one of claim 1-75, and a pharmaceutically acceptable carrier.

77. A pharmaceutical composition, comprising a compound of claim 75, and a pharmaceutically acceptable carrier.

78. A method of inhibiting FGFR2 signaling activity in a subject, comprising administering a therapeutically effective amount of a compound of any one of claim 1-75, or the pharmaceutical composition of any one of claim 76-78, to a subject in need thereof.

79. A method of inhibiting FGFR2 signaling activity in a subject, comprising administering a therapeutically effective amount of a compound of claim 75, or the pharmaceutical composition of any one of claim 76-78, to a subject in need thereof.

80. A method of treating an FGFR2-mediated disorder in a subject, comprising administering a therapeutically effective amount of a compound of any one of claims 1-75, or the pharmaceutical composition of any one of claim 76-78, to a subject in need thereof.

81. A method of treating an FGFR2-mediated disorder in a subject, comprising administering a therapeutically effective amount of a compound of claim 75, or the pharmaceutical composition of any one of claim 76-78, to a subject in need thereof.

82. A method of treating a disorder in a subject, comprising administering a therapeutically effective amount of a compound of any one of claims 1-75, or the pharmaceutical composition of any one of claim 76, to a subject in need thereof, wherein, the disorder is bile duct cancer, liver cancer, breast cancer, prostate cancer, lung cancer, thyroid cancer, gastric cancer, ovarian cancer, rectal cancer, endometrial cancer, or urothelial cancer.

83. A method of treating a disorder in a subject, comprising administering a therapeutically effective amount of a compound of claim 75, or the pharmaceutical composition of any one of claim 76-78, to a subject in need thereof, wherein, the disorder is bile duct cancer, liver cancer, breast cancer, prostate cancer, lung cancer, thyroid cancer, gastric cancer, ovarian cancer, rectal cancer, endometrial cancer, or urothelial cancer.

84. The method of claim 82 or 83, wherein, the disorder is bile duct cancer.

85. The method of claim 84, wherein, the bile duct cancer is intrahepatic cholangiocarcinoma.

86. The method of claim 82 or 83, wherein, the disorder is liver cancer.

87. The method of claim 86, wherein, the liver cancer is hepatocellular carcinoma.

88. The method of claim 82 or 83, wherein, the disorder is lung cancer.

89. The method of claim 88, wherein, the lung cancer is lung squamous cell carcinoma or non-small cell lung cancer.
